SEQUENCE LISTING (295 sequences)

SEQ ID NO: 1 - protein, 126 aa, Homo sapiens
EVQLLESGGGLVQPGGSLRLSCAASGFTFSNYAMNWVRQAPGKGLEWVSAISGGGGSTYYADSVKGRFTISRDNSKNTLYLQMNSLRAEDTAVYYCAKESITMVRGVMDYYGMDVWGQGTTVTVSS

SEQ ID NO: 2 - protein, 10 aa, Homo sapiens
GFTFSNYAMN

SEQ ID NO: 3 - protein, 15 aa, Homo sapiens
AISGGGGSTYYADSV

SEQ ID NO: 4 - protein, 19 aa, Homo sapiens
AKESITMVRGVMDYYGMDV

SEQ ID NO: 5 - protein, 107 aa, Homo sapiens
DIQLTQSPSFLSASVGDRVTITCRASQGISSYLAWYQQKPGKAPKLLIYAASTLQGGVPSRFSGSGSGTEFTLTISSLQPEDFATYYCQHLNRFPRTFGQGTKVEIK

SEQ ID NO: 6 - protein, 11 aa, Homo sapiens
RASQGISSYLA

SEQ ID NO: 7 - protein, 7 aa, Homo sapiens
AASTLQG

SEQ ID NO: 8 - protein, 9 aa, Homo sapiens
QHLNRFPRT

SEQ ID NO: 9 - protein, 836 aa, Homo sapiens
MAGLNCGVSIALLGVLLLGAARLPRGAEAFEIALPRESNITVLIKLGTPTLLAKPCYIVISKRHITMLSIKSGERIVFTFSCQSPENHFVIEIQKNIDCMSGPCPFGEVQLQPSTSLLPTLNRTFIWDVKAHKSIGLELQFSIPRLRQIGPGESCPDGVTHSISGRIDATVVRIGTFCSNGTVSRIKMQEGVKMALHLPWFHPRNVSGFSIANRSSIKRLCIIESVFEGEGSATLMSANYPEGFPEDELMTWQFVVPAHLRASVSFLNFNLSNCERKEERVEYYIPGSTTNPEVFKLEDKQPGNMAGNFNLSLQGCDQDAQSPGILRLQFQVLVQHPQNESNKIYVVDLSNERAMSLTIEPRPVKQSRKFVPGCFVCLESRTCSSNLTLTSGSKHKISFLCDDLTRLWMNVEKTISCTDHRYCQRKSYSLQVPSDILHLPVELHDFSWKLLVPKDRLSLVLVPAQKLQQHTHEKPCNTSFSYLVASAIPSQDLYFGSFCPGGSIKQIQVKQNISVTLRTFAPSFQQEASRQGLTVSFIPYFKEEGVFTVTPDTKSKVYLRTPNWDRGLPSLTSVSWNISVPRDQVACLTFFKERSGVVCQTGRAFMIIQEQRTRAEEIFSLDEDVLPKPSFHHHSFWVNISNCSPTSGKQLDLLFSVTLTPRTVDLTVILIAAVGGGVLLLSALGLIICCVKKKKKKTNKGPAVGIYNDNINTEMPRQPKKFQKGRKDNDSHVYAVIEDTMVYGHLLQDSSGSFLQPEVDTYRPFQGTMGVCPPSPPTICSRAPTAKLATEEPPPRSPPESESEPYTFSHPNNGDVSSKDTDIPLLNTQEPMEPAE

SEQ ID NO: 10 - protein, 455 aa, Homo sapiens
EVQLLESGGGLVQPGGSLRLSCAASGFTFSNYAMNWVRQAPGKGLEWVSAISGGGGSTYYADSVKGRFTISRDNSKNTLYLQMNSLRAEDTAVYYCAKESITMVRGVMDYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 11 - protein, 214 aa, Homo sapiens
DIQLTQSPSFLSASVGDRVTITCRASQGISSYLAWYQQKPGKAPKLLIYAASTLQGGVPSRFSGSGSGTEFTLTISSLQPEDFATYYCQHLNRFPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 5 aa, Homo sapiens
NYAMN

SEQ ID NO: 13 - protein, 17 aa, Homo sapiens
AISGGGGSTYYADSVKG

SEQ ID NO: 14 - protein, 17 aa, Homo sapiens
ESITMVRGVMDYYGMDV

SEQ ID NO: 15 - protein, 117 aa, Homo sapiens
QVQLVQSGAEVKKPGSSVKVSCKASGGTFSSYAMSWVRQAPGQGLEWMGGIIPILGTTNYAQKFQGRVTITADKSTSTAYMELSSLRSEDTAVYYCAREGLYAFDIWGQGTMVTVSS

SEQ ID NO: 16 - protein, 10 aa, Homo sapiens
GGTFSSYAMS

SEQ ID NO: 17 - protein, 13 aa, Homo sapiens
GIIPILGTTNYAQ

SEQ ID NO: 18 - protein, 10 aa, Homo sapiens
AREGLYAFDI

SEQ ID NO: 19 - protein, 107 aa, Homo sapiens
DIQMTQSPSSLSASVGDRVTITCRASQSISSYLNWYQQKPGKAPKFLIYVASSLQSGVPSRFSGRGSGTDFTLTISSLQPEDFATYYCQQSYSTPWTFGQGTKVEIK

SEQ ID NO: 20 - protein, 11 aa, Homo sapiens
RASQSISSYLN

SEQ ID NO: 21 - protein, 7 aa, Homo sapiens
VASSLQS

SEQ ID NO: 22 - protein, 9 aa, Homo sapiens
QQSYSTPWT

SEQ ID NO: 23 - protein, 121 aa, Homo sapiens
QVTLRESGPALVKPTQTLTLTCTFSGFSLSTSGMGVSWIRQPPGKALEWLAHIYWDDDKRYNPSLKSRLTISKDTSKNQVVLTMTNMDPVDTATYYCARSGSYYGSSLDSWGQGTTVTVSS

SEQ ID NO: 24 - protein, 121 aa, Homo sapiens
QVTLRESGPALVKPTQTLTLTCTFSGFSLSTFGMGVSWIRQPPGKALEWLAHIYWDDDKRYNPSLKSRLTISKDTSKNQVVLTMTNMDPVDTATYYCARSGSYYGSSLDSWGQGTTVTVSS

SEQ ID NO: 25 - protein, 116 aa, Homo sapiens
QVQLVQSGAEVKKPGSSVKVSCKASGFNIKDYYMNWVRQAPGQGLEWIGWIDPENANTIYDPKFQGRATITADESTSTAYMELSSLRSEDTAVYYCYGSSYRFTYWGQGTLVTVSS

SEQ ID NO: 26 - protein, 116 aa, Homo sapiens
EVQLVESGGGLVQPGGSLRLSCAASGFTFSSYTMSWVRQAPEKRLEWVAYISSGGGSTYYPDTVKGRFTISRDNAKNSLYLQMNSLRAEDTAVYYCARYDLGFAYWGQGTLVTVSS

SEQ ID NO: 27 - protein, 121 aa, Homo sapiens
QVTLRESGPALVKPTQTLTLTCTFSGFSLSTSGMGVSWIRQPPGKGLEWLAHIYWDDDKRYNPSLKSRLTISKDTSKNQVVLTMTNMDPVDTATYYCARSGSYYGSSLDSWGQGTTVTVSS

SEQ ID NO: 28 - protein, 121 aa, Homo sapiens
QVTLRESGPALVKPTQTLTLTCTFSGFSLSTFGMGVSWIRQPPGKGLEWLAHIYWDDDKRYNPSLKSRLTISKDTSKNQVVLTMTNMDPVDTATYYCARSGSYYGSSLDSWGQGTTVTVSS

SEQ ID NO: 29 - protein, 116 aa, Homo sapiens
EVQLVESGGGLVQPGGSLRLSCAASGFTFSSYTMSWVRQAPGKRLEWVAYISSGGGSTYYPDTVKGRFTISRDNAKNSLYLQMNSLRAEDTAVYYCARYDLGFAYWGQGTLVTVSS

SEQ ID NO: 30 - protein, 116 aa, Homo sapiens
EVQLQESGGGLVQPGGSLRLSCAASGFTFSSYTMSWVRQAPGKRLEWVAYISSGGGSTYYPDTVKGRFTISRDNAKNSLYLQMNSLRAEDTAVYYCARYDLGFAYWGQGTLVTVSS

SEQ ID NO: 31 - protein, 127 aa, Homo sapiens
EVQLVESGGGLVQPGGSLRLSCAASGFNFSSYSIHWVRQAPGKGLEWVASIYPYSGSTYYADSVKGRFTISADTSKNTAYLQMNSLRAEDTAVYYCARQSHSYYSHTWWVSYGMDYWGQGTLVTVSS

SEQ ID NO: 32 - protein, 127 aa, Homo sapiens; identical to SEQ ID NO: 31

SEQ ID NO: 33 - protein, 127 aa, Homo sapiens
EVQLVESGGGLVQPGGSLRLSCAASGFDFSSYSIHWVRQAPGKGLEWVASIYPYSGSTYYADSVKGRFTISADTSKNTAYLQMNSLRAEDTAVYYCARQSHSYYSHTWWVSYGMDYWGQGTLVTVSS

SEQ ID NO: 34 - protein, 127 aa, Homo sapiens
EVQLVESGGGLVQPGGSLRLSCAASGFDFSSYSIHWVRQAPGKGLEWVASIYPYSGSTYYADSVKGRFTISADTSKNTAYLQMNSLRAEDTAVYYCARQSHSYYSHTWWVSYGADYWGQGTLVTVSS

SEQ ID NO: 35 - protein, 127 aa, Homo sapiens
EVQLVESGGGLVQPGGSLRLSCAASGFDFSSYSIHWVRQAPGKGLEWVASIYPYSGSTYYADSVKGRFTISADTSKNTAYLQMNSLRAEDTAVYYCARQSHSYYSHTWWVSYGIDYWGQGTLVTVSS

SEQ ID NO: 36 - protein, 127 aa, Homo sapiens
EVQLVESGGGLVQPGGSLRLSCAASGFDFSSYSIHWVRQAPGKGLEWVASIYPYSGSTYYADSVKGRFTISADTSKNTAYLQMNSLRAEDTAVYYCARQSHSYYSHTWWVSYGLDYWGQGTLVTVSS

SEQ ID NO: 37 - protein, 127 aa, Homo sapiens
EVQLVESGGGLVQPGGSLRLSCAASGFDFSSYSIHWVRQAPGKGLEWVASIYPYSGSTYYADSVKGRFTISADTSKNTAYLQMNSLRAEDTAVYYCARQSHSYYSHTWWVSYGVDYWGQGTLVTVSS

SEQ ID NO: 38 - protein, 129 aa, Homo sapiens
EVQLVESGGGLVQPGGSLRLSCAASGFNFSSSSIHWVRQAPGKGLEWVASIYPYSGSTSYADSVKGRFTISADTSKNTAYLQMNSLRAEDTAVYYCARTYWVQFWYGHFSYYRPALDYWGQGTLVTVSS

SEQ ID NO: 39 - protein, 129 aa, Homo sapiens
EVQLVESGGGLVQPGGSLRLSCAASGFDFSSSSIHWVRQAPGKGLEWVASIYPYSGSTSYADSVKGRFTISADTSKNTAYLQMNSLRAEDTAVYYCARTYWVQFWYGHFSYYRPALDYWGQGTLVTVSS

SEQ ID NO: 40 - protein, 127 aa, Homo sapiens
EVQLVESGGGLVQPGGSLRLSCAASGFDFSSYSIHWVRQAPGKGLEWVASIYPYSGSTYYADSVKGRFTISADTSKNTAYLQMNSLRAEDTAVYYCARQNHSYYSHTWWVSYGMDYWGQGTLVTVSS

SEQ ID NO: 41 - protein, 127 aa, Homo sapiens
EVQLVESGGGLVQPGGSLRLSCAASGFDFSSYSIHWVRQAPGKGLEWVASIYPYSGSTYYADSVKGRFTISADTSKNTAYLQMNSLRAEDTAVYYCARQTHSYYSHTWWVSYGMDYWGQGTLVTVSS

SEQ ID NO: 42 - protein, 127 aa, Homo sapiens
EVQLVESGGGLVQPGGSLRLSCAASGFDFSSYSIHWVRQAPGKGLEWVASIYPYSGSTYYADSVKGRFTISADTSKNTAYLQMNSLRAEDTAVYYCARQGHSYYSHTWWVSYGMDYWGQGTLVTVSS

SEQ ID NO: 43 - protein, 127 aa, Homo sapiens
EVQLVESGGGLVQPGGSLRLSCAASGFDFSSYSIHWVRQAPGKGLEWVASIYPYSGSTYYADSVKGRFTISADTSKNTAYLQMNSLRAEDTAVYYCARQAHSYYSHTWWVSYGMDYWGQGTLVTVSS

SEQ ID NO: 44 - protein, 127 aa, Homo sapiens
EVQLVESGGGLVQPGGSLRLSCAASGFDFSSYSIHWVRQAPGKGLEWVASIYPYSGSTYYADSVKGRFTISADTSKNTAYLQMNSLRAEDTAVYYCARQGQSYYSHTWWVSYGMDYWGQGTLVTVSS

SEQ ID NO: 45 - protein, 127 aa, Homo sapiens
EVQLVESGGGLVQPGGSLRLSCAASGFDFSSYSIHWVRQAPGKGLEWVASIYPYSGSTYYADSVKGRFTISADTSKNTAYLQMNSLRAEDTAVYYCARRSHSYYSHTWWVSYGMDYWGQGTLVTVSS

SEQ ID NO: 46 - protein, 127 aa, Homo sapiens
EVQLVESGGGLVQPGGSLRLSCAASGFDFSSYSIHWVRQAPGKGLEWVASIYPYSGSTYYADSVKGRFTISADTSKNTAYLQMNSLRAEDTAVYYCARQSHSFYSHTWWVSYGMDYWGQGTLVTVSS

SEQ ID NO: 47 - protein, 127 aa, Homo sapiens
EVQLVESGGGLVQPGGSLRLSCAASGFDFSSYSIHWVRQAPGKGLEWVASIYPYSGSTYYADSVKGRFTISADTSKNTAYLQMNSLRAEDTAVYYCARQDHSYYSHTWWVSYGMDYWGQGTLVTVSS

SEQ ID NO: 48 - protein, 127 aa, Homo sapiens
EVQLVESGGGLVQPGGSLRLSCAASGFDFSSYSIHWVRQAPGKGLEWVASIYPYSGSTYYADSVKGRFTISADTSKNTAYLQMNSLRAEDTAVYYCARKYHSYYSHTWWVSYGMDYWGQGTLVTVSS

SEQ ID NO: 49 - protein, 129 aa, Homo sapiens
EVQLVESGGGLVQPGGSLRLSCAASGFNFSSSSIHWVRQAPGKGLEWVASIYPYSGSTSYADSVKGRFTISADTSKNTAYLQMNSLRAEDTAVYYCARIYWVDFWYGHFSYYRPALDYWGQGTLVTVSS

SEQ ID NO: 50 - protein, 127 aa, Homo sapiens
EVQLVESGGGLVQPGGSLRLSCAASGFDASSYSIHWVRQAPGKGLEWVASIYPYSGSTYYADSVKGRFTISADTSKNTAYLQMNSLRAEDTAVYYCARQSHSYYSHTWWVSYGMDYWGQGTLVTVSS

SEQ ID NO: 51 - protein, 127 aa, Homo sapiens
EVQLVESGGGLVQPGGSLRLSCAASGFDFSSYSAHWVRQAPGKGLEWVASIYPYSGSTYYADSVKGRFTISADTSKNTAYLQMNSLRAEDTAVYYCARQSHSYYSHTWWVSYGMDYWGQGTLVTVSS

SEQ ID NO: 52 - protein, 127 aa, Homo sapiens
EVQLVESGGGLVQPGGSLRLSCAASGFDFSSYSIHWVRQAPGKGLEWVASIYPYSGSTSYADSVKGRFTISADTSKNTAYLQMNSLRAEDTAVYYCARQSHSYYSHTWWVSYGMDYWGQGTLVTVSS

SEQ ID NO: 53 - protein, 127 aa, Homo sapiens
EVQLVESGGGLVQPGGSLRLSCAASGFDFSSYSIHWVRQAPGKGLEWVASIYPYSGSTYYADSVKGRFTISADTSKNTAYLQMNSLRAEDTAVYYCARQSHSYYSHTAWVSYGMDYWGQGTLVTVSS

SEQ ID NO: 54 - protein, 127 aa, Homo sapiens
EVQLVESGGGLVQPGGSLRLSCAASGFDFSSYSIHWVRQAPGKGLEWVASSYPYSGSTYYADSVKGRFTISADTSKNTAYLQMNSLRAEDTAVYYCARQSHSYYSHTWWVSYGMDYWGQGTLVTVSS

SEQ ID NO: 55 - protein, 233 aa, Homo sapiens
EISEVQLVESGGGLVQPGGSLRLSCAASGFNFSSYSIHWVRQAPGKGLEWVASIYPYSGSTYYADSVKGRFTISADTSKNTAYLQMNSLRAEDTAVYYCARQSHSYYSHTWWVSYGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 56 - protein, 457 aa, Homo sapiens
EVQLVESGGGLVQPGGSLRLSCAASGFNFSSYSIHWVRQAPGKGLEWVASIYPYSGSTYYADSVKGRFTISADTSKNTAYLQMNSLRAEDTAVYYCARQSHSYYSHTWWVSYGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 457 aa, Homo sapiens
EVQLVESGGGLVQPGGSLRLSCAASGFDFSSYSIHWVRQAPGKGLEWVASIYPYSGSTYYADSVKGRFTISADTSKNTAYLQMNSLRAEDTAVYYCARQSHSYYSHTWWVSYGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - protein, 457 aa, Homo sapiens
EVQLVESGGGLVQPGGSLRLSCAASGFDFSSYSIHWVRQAPGKGLEWVASIYPYSGSTYYADSVKGRFTISADTSKNTAYLQMNSLRAEDTAVYYCARQSHSYYSHTWWVSYGADYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - protein, 457 aa, Homo sapiens
EVQLVESGGGLVQPGGSLRLSCAASGFDFSSYSIHWVRQAPGKGLEWVASIYPYSGSTYYADSVKGRFTISADTSKNTAYLQMNSLRAEDTAVYYCARQSHSYYSHTWWVSYGIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 457 aa, Homo sapiens
EVQLVESGGGLVQPGGSLRLSCAASGFDFSSYSIHWVRQAPGKGLEWVASIYPYSGSTYYADSVKGRFTISADTSKNTAYLQMNSLRAEDTAVYYCARQSHSYYSHTWWVSYGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 457 aa, Homo sapiens
EVQLVESGGGLVQPGGSLRLSCAASGFDFSSYSIHWVRQAPGKGLEWVASIYPYSGSTYYADSVKGRFTISADTSKNTAYLQMNSLRAEDTAVYYCARQSHSYYSHTWWVSYGVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 235 aa, Homo sapiens
EISEVQLVESGGGLVQPGGSLRLSCAASGFNFSSSSIHWVRQAPGKGLEWVASIYPYSGSTSYADSVKGRFTISADTSKNTAYLQMNSLRAEDTAVYYCARTYWVQFWYGHFSYYRPALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 63 - protein, 459 aa, Homo sapiens
EVQLVESGGGLVQPGGSLRLSCAASGFNFSSSSIHWVRQAPGKGLEWVASIYPYSGSTSYADSVKGRFTISADTSKNTAYLQMNSLRAEDTAVYYCARTYWVQFWYGHFSYYRPALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 64 - protein, 459 aa, Homo sapiens
EVQLVESGGGLVQPGGSLRLSCAASGFDFSSSSIHWVRQAPGKGLEWVASIYPYSGSTSYADSVKGRFTISADTSKNTAYLQMNSLRAEDTAVYYCARTYWVQFWYGHFSYYRPALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - protein, 457 aa, Homo sapiens; identical to SEQ ID NO: 57

SEQ ID NO: 66 - protein, 457 aa, Homo sapiens
EVQLVESGGGLVQPGGSLRLSCAASGFDFSSYSIHWVRQAPGKGLEWVASIYPYSGSTYYADSVKGRFTISADTSKNTAYLQMNSLRAEDTAVYYCARQNHSYYSHTWWVSYGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67 - protein, 457 aa, Homo sapiens
EVQLVESGGGLVQPGGSLRLSCAASGFDFSSYSIHWVRQAPGKGLEWVASIYPYSGSTYYADSVKGRFTISADTSKNTAYLQMNSLRAEDTAVYYCARQTHSYYSHTWWVSYGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - protein, 457 aa, Homo sapiens
EVQLVESGGGLVQPGGSLRLSCAASGFDFSSYSIHWVRQAPGKGLEWVASIYPYSGSTYYADSVKGRFTISADTSKNTAYLQMNSLRAEDTAVYYCARQGHSYYSHTWWVSYGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - protein, 457 aa, Homo sapiens
EVQLVESGGGLVQPGGSLRLSCAASGFDFSSYSIHWVRQAPGKGLEWVASIYPYSGSTYYADSVKGRFTISADTSKNTAYLQMNSLRAEDTAVYYCARQAHSYYSHTWWVSYGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - protein, 457 aa, Homo sapiens
EVQLVESGGGLVQPGGSLRLSCAASGFDFSSYSIHWVRQAPGKGLEWVASIYPYSGSTYYADSVKGRFTISADTSKNTAYLQMNSLRAEDTAVYYCARQGQSYYSHTWWVSYGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 457 aa, Homo sapiens; identical to SEQ ID NO: 57

SEQ ID NO: 72 - protein, 457 aa, Homo sapiens; identical to SEQ ID NO: 57

SEQ ID NO: 73 - protein, 457 aa, Homo sapiens; identical to SEQ ID NO: 57

SEQ ID NO: 74 - protein, 457 aa, Homo sapiens
EVQLVESGGGLVQPGGSLRLSCAASGFDFSSYSIHWVRQAPGKGLEWVASIYPYSGSTYYADSVKGRFTISADTSKNTAYLQMNSLRAEDTAVYYCARRSHSYYSHTWWVSYGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 75 - protein, 457 aa, Homo sapiens; identical to SEQ ID NO: 57

SEQ ID NO: 76 - protein, 457 aa, Homo sapiens
EVQLVESGGGLVQPGGSLRLSCAASGFDFSSYSIHWVRQAPGKGLEWVASIYPYSGSTYYADSVKGRFTISADTSKNTAYLQMNSLRAEDTAVYYCARQSHSFYSHTWWVSYGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - protein, 457 aa, Homo sapiens; identical to SEQ ID NO: 74

SEQ ID NO: 78 - protein, 457 aa, Homo sapiens
EVQLVESGGGLVQPGGSLRLSCAASGFDFSSYSIHWVRQAPGKGLEWVASIYPYSGSTYYADSVKGRFTISADTSKNTAYLQMNSLRAEDTAVYYCARQDHSYYSHTWWVSYGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - protein, 457 aa, Homo sapiens; identical to SEQ ID NO: 74

SEQ ID NO: 80 - protein, 457 aa, Homo sapiens; identical to SEQ ID NO: 74

SEQ ID NO: 81 - protein, 457 aa, Homo sapiens; identical to SEQ ID NO: 57

SEQ ID NO: 82 - protein, 457 aa, Homo sapiens; identical to SEQ ID NO: 74

SEQ ID NO: 83 - protein, 457 aa, Homo sapiens; identical to SEQ ID NO: 57

SEQ ID NO: 84 - protein, 457 aa, Homo sapiens; identical to SEQ ID NO: 57

SEQ ID NO: 85 - protein, 457 aa, Homo sapiens; identical to SEQ ID NO: 57

SEQ ID NO: 86 - protein, 457 aa, Homo sapiens
EVQLVESGGGLVQPGGSLRLSCAASGFDFSSYSIHWVRQAPGKGLEWVASIYPYSGSTYYADSVKGRFTISADTSKNTAYLQMNSLRAEDTAVYYCARKYHSYYSHTWWVSYGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 87 - protein, 457 aa, Homo sapiens; identical to SEQ ID NO: 78

SEQ ID NO: 88 - protein, 457 aa, Homo sapiens; identical to SEQ ID NO: 74

SEQ ID NO: 89 - protein, 457 aa, Homo sapiens; identical to SEQ ID NO: 74

SEQ ID NO: 90 - protein, 457 aa, Homo sapiens; identical to SEQ ID NO: 57

SEQ ID NO: 91 - protein, 459 aa, Homo sapiens
EVQLVESGGGLVQPGGSLRLSCAASGFNFSSSSIHWVRQAPGKGLEWVASIYPYSGSTSYADSVKGRFTISADTSKNTAYLQMNSLRAEDTAVYYCARIYWVDFWYGHFSYYRPALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 92 - protein, 118 aa, Homo sapiens
EVQLQQFGAELVKPGASVKISCKASGYSFSDFNIEWLKQSHGKSLEWIGDINPNYDSTNYNQKFKGRATLTVDKSSSTAYMEVRSLTSEDTAVYYCARLGYGYAMDYWGQGTSVTVSS

SEQ ID NO: 93 - protein, 448 aa, Homo sapiens
EVQLQQFGAELVKPGASVKISCKASGYSFSDFNIEWLKQSHGKSLEWIGDINPNYDSTNYNQKFKGRATLTVDKSSSTAYMEVRSLTSEDTAVYYCARLGYGYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 94 - protein, 120 aa, Homo sapiens
QVTLKESGPGILQPSQTLSLTCSFSGFSLSTAGVGVSWIRQPSGKGLEWLAHIYWDDDKRYNPSLKSRLTISKDTSRNQVFLKITSVDSADTATYFCSRSTSVEEAFDVWGAGTTVTVSS

SEQ ID NO: 95 - protein, 119 aa, Homo sapiens
QVTLKESGPGILQSSQTLSLTCSFSGFSLSTAGMGVSWIRQPSGKGLEWLAHIYWDDDKRYNPSLKSRLTVSKGTSRNQVLLKITSVDAADTATYYCARSDSFGDFDYWGQGTTLTVSS

SEQ ID NO: 96 - protein, 118 aa, Homo sapiens
QVHLKQSGPGLVQPSQSLSITCTVSGFSLTNFGLHWVRQSPGKGLEWLGVIWSGGVTDYNAAFISRLSISKDNSKSQVFFKMNSLQPNDTAIYYCARNYDHDYSMDYWGQGISVSVSS

SEQ ID NO: 97 - protein, 128 aa, Homo sapiens
EVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDGVLRYFDWLLDYYYYMDVWGKGTTVTVSS

SEQ ID NO: 98 - protein, 128 aa, Homo sapiens
EVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDGELRHFDHLLDYHYYMDVWGQGTTVTVSS

SEQ ID NO: 99 - protein, 128 aa, Homo sapiens; identical to SEQ ID NO: 98

SEQ ID NO: 100 - protein, 128 aa, Homo sapiens
EVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDAELRHFDHLLDYHYYMDVWGQGTTVTVSS

SEQ ID NO: 101 - protein, 128 aa, Homo sapiens
EVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDGELRHFDQVYNYHYYMDVWGQGTTVTVSS

SEQ ID NO: 102 - protein, 106 aa, Homo sapiens
EIVLTQSPATLSLSPGERATLSCRASQSVGSYLAWYQQRPGQAPRLLIYDASNRATGIPARFSGSGSGTDFTLTISSLEPEDFAVYYCQQRANVFTFGQGTKVEIK

SEQ ID NO: 103 - protein, 116 aa, Homo sapiens
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARGVGLPDIWGQGTMVTVSS

SEQ ID NO: 104 - protein, 126 aa, Homo sapiens
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARDPTYFDWTRRGYYYMDVWGKGTTVTVSS

SEQ ID NO: 105 - protein, 115 aa, Homo sapiens
EVQLLESGGDLVQPGGSLRLSCAASGFTFNSYAMSWVRQAPGKGLEWVSVLSGSGGDIHYADSVKGRFTVSRDNSKNMLYLQMNSLRAEDTAVYFCAQQWPQGYWGQGTLVTVSS

SEQ ID NO: 106 - protein, 122 aa, Homo sapiens
QVQLVESGGGVVQPGRSLRLSCAASGFTFSSYGMHWVRQAPGKGLEWVAIIWYDGSNKYYADSVKGRFTISRDNSKNTLYLQMNSLRAEDTAVYYCAKDPSYSSGYYLFDFWGQGTLVTVSS

SEQ ID NO: 107 - protein, 115 aa, Homo sapiens
EVQVLESGGGLVQPGGSLRLSCAASGFTFSSYVMSWVRQAPGKGLEWVSGISGSGGSTHYADSVKGRFTISRDNSKNTLYLQMNSLRAEDTALYYCAQQWPQGYWGQGTLVTVSS

SEQ ID NO: 108 - protein, 117 aa, Homo sapiens; identical to SEQ ID NO: 15

SEQ ID NO: 109 - protein, 114 aa, Homo sapiens
QVQLQQWDAGLLKPSETLSLTCAVYGGSFSSYYWSWIRQPPGKGLEWIGEINHSGSTSYNPSLKSRVTISIDTSKNQFSLKLNSMTAADTAVYFCAASPYFDYWGQGTLVTVSS

SEQ ID NO: 110 - protein, 116 aa, Homo sapiens
QVQLVESGGGVVQPGRSLRLSCVVSGFTLSSYGMHWVRQAPGKGLEWVAVIWYDGSDKYYADSVKGRFTISRDNSKNTLYLQMNSLRAEDTAVYYCARDDWGFDYWGQGTLVTVSS

SEQ ID NO: 111 - protein, 120 aa, Homo sapiens
EVQLVESGGGLVQPGGSLRLSCAASGFTFSRYWMNWVRQAPGKGLEWVANIKQDGSEKDYVDSVKGRFTISRDNAKNSLFLQMSSLRAEDTAVYYCARVMYSSGWSFDYWGQGTLVTVSS

SEQ ID NO: 112 - protein, 120 aa, Homo sapiens
EVQLVESGGGLVQPGGSLRLSCAASGFTFSSYWMSWVRQAPGKGLEWVANIKQDGSEKDYVDSVKGRFTISRDNAKNSLYLQMNSLRVEDTAVYYCAREGGSSGWTFDYWGQGTLVTVSS

SEQ ID NO: 113 - protein, 118 aa, Homo sapiens
QVQLQESGPGQVKPSETLSLTCTVSGGSISSSFWSWIRQPPGKGLEWIGYIYYSESTNYNPSLKRRVTLSVDTSKNQFSLKLTSVTTADTAVYYCARNIGVAGLFDYWGQGTLVTVSS

SEQ ID NO: 114 - protein, 118 aa, Homo sapiens
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCARNIGVAGLFDYWGQGTLVTVSS

SEQ ID NO: 115 - protein, 122 aa, Homo sapiens
QVQLVESGGGVVQPGRSLRLSCAASGFTFSSYGMHWVRQAPGKGLEWVAVVWYDGTIKYYADSVKGRFTISRDNPKNTLYLQMNSLRAEDTAVYYCASQYSSGWHTDFFDVWGQGTMVTVSS

SEQ ID NO: 116 - protein, 114 aa, Homo sapiens
QVQLVESGGGVVQPGRSLRLSCAASGFTFSSYGMHWVRQAPGKGLEWVAVIWYAGSNKYYADSVKGRFTISRDNSKNTLYLQMNSLRAEDTAVYYCAGWDFDYWGQGTLVTVSS

SEQ ID NO: 117 - protein, 125 aa, Homo sapiens
QVQLQQWGAGLLKPSETLSLTCAVYGGSFSDYYWSWIRQPPGKGLEWIGEINHSGSTNYNPSLKSRVTISVDTSKNQFSLKLSSVTAADTAVYYCARDYDVLTGHFYYYYGMDVWGQGTTVTVSS

SEQ ID NO: 118 - protein, 128 aa, Homo sapiens
QVQLVESGGGVVQPGRSLRLSCAASGFSFSDYGIHWVRQAPGKGLEWVAVIWYDGSNKYYADSVKGRFTISRDNSKNTLYLQVNSLIAEDTAVYYCARDRGYSSGWYVDYYYYGMDVWGQGTTVTVSS

SEQ ID NO: 119 - protein, 126 aa, Homo sapiens
EVQVLESGGGLVQPGGSLRLSCAASGFTFSNYAMNWVRQAPGKGLEWVSAISGGGGSTYYADSVKGRATISRDNSENTLYLQMNSLRAEDTAVYYCAKTSSGWYDSYYDYYGLDVWGQGTTVTVSS

SEQ ID NO: 120 - protein, 126 aa, Homo sapiens; identical to SEQ ID NO: 1

SEQ ID NO: 121 - protein, 124 aa, Homo sapiens
EVQLLESGGGLVQPGGSLRLSCAASGFTFSNYAMSWVRQAPGKGLEWVSAISGRGGSTYYADSVKGRFTISRDNSKNTLYLQMNSLRAEDTAVYYCAKDIVVVPAAKGYVMDAWGQGASVTVSS

SEQ ID NO: 122 - protein, 126 aa, Homo sapiens
QEQLVESGGGVVQPGRSLRLSCVTSGFTFSSYGMHWVRQAPGKGLEWVAVIWYDGTNKYYADTVKGRFTISRDNSKNTLYLQMNSLRAEDTGVYYCAREGCDTISCPYYYYGMDVWGQGTTITVSS

SEQ ID NO: 123 - protein, 121 aa, Homo sapiens
EVQLLESGGGLVQPGGSLRLSCAASGFTFSSYAMSWVRQAPGKGLEWVSAISGSGNSAYYADSVKGRFTISRDNSKNTLFLQMNSLRADDTAVYYCASSSGWYLVYYFDLWGRGTLVTVSS

SEQ ID NO: 124 - protein, 115 aa, Homo sapiens
EVQLLESGGGLVQPGGSLRLSCAASGFTFSSYAMSWVRQAPGKGLEWVSGISGSGGNTHYADSVKGRFTISRDNSKNTLYLQMNSLRAEDTALYYCAQQWPQGYWGQGTLVTVSS

SEQ ID NO: 125 - protein, 115 aa, Homo sapiens
EVQLLESGGGLVQPGGSLGLSCAASGFTFSNYIMSWVRQAPGKGLEWVSGISGSGGSTHYAGSVKGRFTISRDNSKNTLNLQMNSLRVEDTAVYHCVQQWPQGYWGQGTLVTVSS

SEQ ID NO: 126 - protein, 115 aa, Homo sapiens
EVQLLESGGGLVQPGGSLRLSCAASGFTFSSYAMSWVRLAPGKGLEWVSGLSGSGGDTHYAGSVKGRFTISRDNSKNTLYLQMNSLRAEDTALYYCAQQWPQGYWGQGTLVTVSS

SEQ ID NO: 127 - protein, 115 aa, Homo sapiens
EVQLLESGGGLVQPGESLRLSCAASGFTFSSYAMSWVRQAPGKGLEWVSGISGSGGSTHYADSVKGRFTISRDNSKNTLYLQMNSLRAEDTAAYYCAQQWPQGHWGQGILVTVSS

SEQ ID NO: 128 - protein, 115 aa, Homo sapiens
EVQLLESGGGLVQPGGSLRLSCAASGFTFSSYAMSWVRQAPGKGLEWVSVLSGSGDDTHYADSVKGRFTISRDNSKNTLYLQMNSLRAEDTAVYYCAHQWPQGYWGQGTLVTVSS

SEQ ID NO: 129 - protein, 115 aa, Homo sapiens
EVQLLESGGGLVQPGGSLRLSCAASGFTFSSYVMSWVRQTPGKGLEWVSGISGSGGSTHYTDSVQGRFTISRDNSKNTLYLQMNSLRAEDTAVYYCVQQWPQGYWGQGTLVTVSS

SEQ ID NO: 130 - protein, 119 aa, Homo sapiens
EVQVLESGGGLVQPGESLRLSCAASGFTFNTYAMSWVRQAPGKGLEWVSAISDNGGGTYNADSVKGRFTISRDNSKNTLYLQMNSLRAEDTAVYYCAKGEQWGAPFDYWGQGTLVTVSS

SEQ ID NO: 131 - protein, 121 aa, Homo sapiens
EVQLLESGGGLVQPGGSLRLSCAASGFTFSSYAMSWVRQAPGKGLEWVSTISGSGGSTYYADSVKGRFTISRDNSKNTLYLQMNSLRAEDTAVYYCATLDTAMAADAFAIWGQGTMVTVSS

SEQ ID NO: 132 - protein, 120 aa, Homo sapiens
EVQLVESGGGLVQPGGSLRLSCAASGFTFSSYWMSWVRQVPGKGLEWVANIKQDGSEKYYVDSVKGRFTISRDNAKNSLYLQMNSLRAEDTAVYYCARDWRSSGWTLDYWGQGTLVTVSS

SEQ ID NO: 133 - protein, 120 aa, Homo sapiens
EVQLVESGGGLVQPGGSLRLSCAASGFTFSSYWMSWVRQAPGKGLEWVANVKQDGSEKDYVDSVKGRFTISRDNAKNSLYLQMNSLRAEDTAVYYCAREWNSSGWTFDYWGQGTLVTVSS

SEQ ID NO: 134 - protein, 128 aa, Homo sapiens
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYHCARDRVYYDGSGSYYNVGVMDVWGQGASVTVSS

SEQ ID NO: 135 - protein, 124 aa, Homo sapiens
QVQLVESGGGVVQPGGSPRLSCAASGFTFSSYAMHWVRQAPGTGLEWVALIYYDGSHEYYSDSVKGRFTISRDNSKNTLYLQMSSLRAEDTAVYYCARDGGSGSHYPFDAFDIWGQGTMVTVSS

SEQ ID NO: 136 - protein, 118 aa, Homo sapiens
QVQLQESGPGLVKPSETLSLTCTVSGGSISSYYWSWIRQFSGKGLEWIGYIYYTGRNNYNPSLKSRVTISVDTSKNQFSLKLSSVTAADSAVYYCAREGGWGPHFDYWGQGTLVTVSS

SEQ ID NO: 137 - protein, 125 aa, Homo sapiens; identical to SEQ ID NO: 117

SEQ ID NO: 138 - protein, 124 aa, Homo sapiens
QVQLVESGGGVVQPGRSLRLSCAASGFTFSSYGMHWVRQAPGKGLEWVALIWYDGSNKYYADSVQGRFTISRDNSKNTLYLQMNSLRAEDTAVYYCAVDYDILTGHVLYVMDAWGQGVSVTVSS

SEQ ID NO: 139 - protein, 122 aa, Homo sapiens
QVQLVESGGGVVQPGRSLRLSCAASGFTFSNYGMNWVRQAPGKGLEWVAVIWYDGTIKYYADSVKGRFTISRDNSKNTLYLQMISLRAEDTAVYYCASEYSSGWYRGAFDIWGQGTMVTVSS

SEQ ID NO: 140 - protein, 125 aa, Homo sapiens
QVQLVESGGGVVQPGRSLRLSCAASGFTFSSYGMFWVRQTPGKGLEWVANIWYDGSNKYYTDSVKGRFTISRDNSKNTLYLQMNSLRAGDTAVYYCARETYYYGSGSYGGGLDVWGQGTTVTVSS

SEQ ID NO: 141 - protein, 119 aa, Homo sapiens
QVQLQESGPGLVKPSETLSLTCAVSGYSISSGYYWGWIRQPPGKGLEWIGSIYHSGSTYYNPSLKSRVTISVDTSKNQFSLKLSSVTAADTAVYYCARDKITVAAFDIWGQGTMVTVSS

SEQ ID NO: 142 - protein, 125 aa, Homo sapiens
QVQLQESGPGLVKPSETLSLTCAVSGYSISSDYYWGWIRQPPGKGLEWIGSIYHSGSTYYNTSLKSRVTISLDTSKNQFSLKLTSVTAADTAVYYCVREGTVGGHYYYYYGMDVWGQGTTVTVSS

SEQ ID NO: 143 - protein, 120 aa, Homo sapiens
EVHLLESGGGLVQPGGSLRLSCAASGFTFSSYAMSWVRQAPGKGLEWVSGIDSGGDTYYADSVKGRFTISRDNSKNTLYLQMTSLRAEDTAVYYCAKDLYSSGWLAFDIWGQGTMVTVSS

SEQ ID NO: 144 - protein, 120 aa, Homo sapiens
EVQLLESGGGSVQPGGSLRLSCAASGFTFSSYAMSWVRQAPGKGLEWVSGISDNGNTYYADSVKGRFTISRDNSKNTLYLQMNSLRAEDTAVYHCAKDLYSSGWLAFDIWGQGTMVTVSS

SEQ ID NO: 145 - protein, 119 aa, Homo sapiens
VQLQQPGAELVKPGASVKMSCKASGYTFTSYYMYWVKQRPGQGLEWIGEINPSHGGTNFNEKFKNKATLTVDKSSSTVYMQLSSLTSEDSAVYYCTRGGNYPYFAMDYWGQGTSVTVSS

SEQ ID NO: 146 - protein, 449 aa, Homo sapiens
VQLQQPGAELVKPGASVKMSCKASGYTFTSYYMYWVKQRPGQGLEWIGEINPSHGGTNFNEKFKNKATLTVDKSSSTVYMQLSSLTSEDSAVYYCTRGGNYPYFAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 147 - protein, 120 aa, Homo sapiens
EVQLVESGGGLVQPGGSLRLSCAASGFTFNSYGMSWVRQAPGKGLEWVATISSGGSYKYYVDSVKGRFTISRDNAKNSLYLQMNSLRAEDTAVYYCARHPDYDGVWFAYWGQGTLVTVSS

SEQ ID NO: 148 - protein, 120 aa, Homo sapiens
EVQLVESGGGLVQPGGSLRLSCAASGFTFNSYGMSWVRQAPGKGLEWVATISSGGSYTYYPDSVKGRFTISRDNAKNSLYLQMNSLRAEDTAVYYCARHPDYDGVWFAYWGQGTLVTVSS

SEQ ID NO: 149 - protein, 120 aa, Homo sapiens
EVQLVESGGGLVKPGGSLRLSCAASGFTFNSYGMSWVRQAPGKGLEWVSTISSGGSYTYYPDSVKGRFTISRDNAKNSLYLQMNSLRAEDTAVYYCARHPDYDGVWFAYWGQGTLVTVSS

SEQ ID NO: 150 - protein, 120 aa, Homo sapiens
EVQLLESGGGLVQPGGSLRLSCAASGFTFNSYGMSWVRQAPGKGLEWVSTISSGGSYTYYPDSVKGRFTISRDNSKNTLYLQMNSLRAEDTAVYYCAKHPDYDGVWFAYWGQGTLVTVSS

SEQ ID NO: 151 - protein, 120 aa, Homo sapiens
EVQLLESGGGLVQPGGSLRLSCAASGFTFNSYGMSWVRQAPGKGLEWVSTISSGGSYTYYPDSVKGRFTISRDNSKNTLYLQMNSLRAEDTAVYYCARHPDYDGVWFAYWGQGTLVTVSS

SEQ ID NO: 152 - protein, 120 aa, Homo sapiens
EVQLVESGGGLVKPGGSLRLSCAASGFTFNSYGMSWVRQAPGKGLEWVSSISSGGSYIYYADSVKGRFTISRDNAKNSLYLQMNSLRAEDTAVYYCARHPDYDGVWFAYWGQGTLVTVSS

SEQ ID NO: 153 - protein, 120 aa, Homo sapiens
EVQLVESGGGLVKPGGSLRLSCAASGFTFNSYGMSWVRQAPGKGLEWVSSISSGGSYTYYADSVKGRFTISRDNAKNSLYLQMNSLRAEDTAVYYCARHPDYDGVWFAYWGQGTLVTVSS

SEQ ID NO: 154 - protein, 120 aa, Homo sapiens
EVQLLESGGGLVQPGGSLRLSCAASGFTFNSYAMSWVRQAPGKGLEWVSAISSGGSYTYYADSVKGRFTISRDNSKNTLYLQMNSLRAEDTAVYYCAKHPDYDGVWFAYWGQGTLVTVSS

SEQ ID NO: 155 - protein, 120 aa, Homo sapiens
EVQLLESGGGLVQPGGSLRLSCAASGFTFNSYAMSWVRQAPGKGLEWVSTISSGGSYTYYPDSVKGRFTISRDNSKNTLYLQMNSLRAEDTAVYYCAKHPDYDGVWFAYWGQGTLVTVSS

SEQ ID NO: 156 - protein, 120 aa, Homo sapiens
EVQLLESGGGLVQPGGSLRLSCAASGFTFNSYGMSWVRQAPGKGLEWVSAISSGGSYTYYPDSVKGRFTISRDNSKNTLYLQMNSLRAEDTAVYYCAKHPDYDGVWFAYWGQGTLVTVSS

SEQ ID NO: 157 - protein, 120 aa, Homo sapiens
EVQLVESGGGLVKPGGSLRLSCAASGFTFNSYSMNWVRQAPGKGLEWVSSISSGGSYIYYADSVKGRFTISRDNAKNSLYLQMNSLRAEDTAVYYCARHPDYDGVWFAYWGQGTLVTVSS

SEQ ID NO: 158 - protein, 120 aa, Homo sapiens
QVTLKESGPGILQPSQTLSLTCSFSGFSLSTSGMGVSWIRQPSGKGLEWLAHIYWDDDKRYNPSLKSRLTISKDTSRNQVFLKITSVDTADTATYYCARSSSYYDCFAYWGQGTLVTVSA

SEQ ID NO: 159 - protein, 112 aa, Homo sapiens
DIVMTQSPLSLPVTPGEPASISCRSSQSIVHSSGNTYLEWYLQKPGQSPQLLIYKVSNRFSGVPDRFSGSGSGTDFTLKISRVEAEDVGVYYCFQGSHVPWTFGGGTKVEIK

SEQ ID NO: 160 - protein, 112 aa, Homo sapiens
DIVMTQSPLSLPVTPGEPASISCRSSQSIVYSSGNTYLEWYLQKPGQSPQLLIYKVSNRFSGVPDRFSGSGSGTDFTLKISRVEAEDVGVYYCFQGSHVPWTFGGGTKVEIK

SEQ ID NO: 161 - protein, 112 aa, Homo sapiens
DIVMTQSPLSLPVTPGEPASISCRSSQNIVHSDGNTYLEWYLQKPGQSPQLLIYKVSNRFSGVPDRFSGSGSGTDFTLKISRVEAEDVGVYYCFQGSHVPWTFGGGTKVEIK

SEQ ID NO: 162 - protein, 112 aa, Homo sapiens; identical to SEQ ID NO: 159

SEQ ID NO: 163 - protein, 106 aa, Homo sapiens
EIVLTQSPATLSLSPGERATLSCSASSSVSYLYWYQQKPGQAPRPWIYLTSNLASGVPARFSGSGSGTDFTLTISSLEPEDFAVYYCQQWSSNPFTFGGGTKVEIK

SEQ ID NO: 164 - protein, 106 aa, Homo sapiens
EIVLTQSPATLSLSPGERATLSCSASSSVSYLYWYQQKPGQAPRPWIYLTSNLASGVPARFSGSGSGTDYTLTISSLEPEDFAVYYCQQWSSNPFTFGGGTKVEIK

SEQ ID NO: 165 - protein, 106 aa, Homo sapiens
EIVLIQSPATLSLSPGERATLSCSASSSVSYLYWYQQKPGQAPRPWIYLTSNLASGVPARFSGSGSGTDFTLTISSLEPEDFAVYYCQQWSSNPFTFGGGTKVEIK

SEQ ID NO: 166 - protein, 106 aa, Homo sapiens; identical to SEQ ID NO: 164

SEQ ID NO: 167 - protein, 106 aa, Homo sapiens; identical to SEQ ID NO: 165

SEQ ID NO: 168 - protein, 111 aa, Homo sapiens
EIVLTQSPATLSLSPGERATLSCRASESVDSYGNSFMHWYQQKPGQAPRLLIFLASNLESGVPARFSGSGSRTDFTLTISSLEPEDFAVYYCQQNNEDPWTFGGGTKVEIK

SEQ ID NO: 169 - protein, 111 aa, Homo sapiens
EIVLTQSPATLSLSPGERATLSCRASESVDSYGNSFMHWYQQKPGQAPRLLIFLASNLESGVPARFSGSGSGTDFTLTISSLEPEDFAVYYCQQNNEDPWTFGGGTKVEIK

SEQ ID NO: 170 - protein, 107 aa, Homo sapiens
DIQMTQSPSSLSASVGDRVTITCRASQSVSSAVAWYQQKPGKAPKLLIYSASSLYSGVPSRFSGSRSGTDFTLTISSLQPEDFATYYCQQSGQRPITFGQGTKVEIK

SEQ ID NO: 171 - protein, 107 aa, Homo sapiens
DIQMTQSPSSLSASVGDRVTITCRASQSVSSAVAWYQQKPGKAPKLLIYSASSLYSGVPSRFSGSRSGTDFTLTISSLQPEDFATYYCQQSSSSLITFGQGTKVEIK

SEQ ID NO: 172 - protein, 107 aa, Homo sapiens
DIQMTQSPSSLSASVGDRVTITCRASQSVSSAVAWYQQKPGKAPKLLIYSASSLYSGVPSRFSGSRSGTDFTLTISSLQPEDFATYYCQQSGERPITFGQGTKVEIK

SEQ ID NO: 173 - protein, 107 aa, Homo sapiens
DIQMTQSPSSLSASVGDRVTITCRASQSVSSAVAWYQQKPGKAPKLLIYSASSLYSGVPSRFSGSRSGTDFTLTISSLQPEDFATYYCQQSGNVPATFGQGTKVEIK

SEQ ID NO: 174 - protein, 107 aa, Homo sapiens
DIQMTQSPSSLSASVGDRVTITCRASQSVSSAVAWYQQKPGKAPKLLIYSASSLYSGVPSRFSGSRSGTDFTLTISSLQPEDFATYYCQQSGKRPMTFGQGTKVEIK

SEQ ID NO: 175 - protein, 107 aa, Homo sapiens
DIQMTQSPSSLSASVGDRVTITCRASQSVSSAVAWYQQKPGKAPKLLIYSASSLYSGVPSRFSGSRSGTDFTLTISSLQPEDFATYYCQQTGQRPMTFGQGTKVEIK

SEQ ID NO: 176 - protein, 107 aa, Homo sapiens
DIQMTQSPSSLSASVGDRVTITCRASQSVSSAVAWYQQKPGKAPKLLIYSASSLYSGVPSRFSGSRSGTDFTLTISSLQPEDFATYYCQQSMQWPKTFGQGTKVEIK

SEQ ID NO: 177 - protein, 107 aa, Homo sapiens
DIQMTQSPSSLSASVGDRVTITCRASQSVSSAVAWYQQKPGKAPKLLIYSASSLYSGVPSRFSGSRSGTDFTLTISSLQPEDFATYYCQQSLQRPMTFGQGTKVEIK

SEQ ID NO: 178 - protein, 107 aa, Homo sapiens
DIQMTQSPSSLSASVGDRVTITCRASQSVSSAVAWYQQKPGKAPKLLIYSASSLYSGVPSRFSGSRSGTDFTLTISSLQPEDFATYYCQQVELVPMTFGQGTKVEIK

SEQ ID NO: 179 - protein, 107 aa, Homo sapiens
DIQMTQSPSSLSASVGDRVTITCRASQSVSSAVAWYQQKPGKAPKLLIYSASSLYSGVPSRFSGSRSGTDFTLTISSLQPEDFATYYCQQAGKRPLTFGQGTKVEIK

SEQ ID NO: 180 - protein, 107 aa, Homo sapiens
DIQMTQSPSSLSASVGDRVTITCRASQSVSSAVAWYQQKPGKAPKLLIYSASSLYSGVPSRFSGSRSGTDFTLTISSLQPEDFATYYCQQSVDLTATFGQGTKVEIK

SEQ ID NO: 181 - protein, 107 aa, Homo sapiens
DIQMTQSPSSLSASVGDRVTITCRASQSVSSAVAWYQQKPGKAPKLLIYSASSLYSGVPSRFSGSRSGTDFTLTISSLQPEDFATYYCQQLGVRAATFGQGTKVEIK

SEQ ID NO: 182 - protein, 107 aa, Homo sapiens
DIQMTQSPSSLSASVGDRVTITCRASQSVSSAVAWYQQKPGKAPKLLIYSASSLYSGVPSRFSGSRSGTDFTLTISSLQPEDFATYYCQQSGRKPVTFGQGTKVEIK

SEQ ID NO: 183 - protein, 107 aa, Homo sapiens
DIQMTQSPSSLSASVGDRVTITCRASQSVSSAVAWYQQKPGKAPKLLIYSASSLYSGVPSRFSGSRSGTDFTLTISSLQPEDFATYYCQQTAQSPLTFGQGTKVEIK

SEQ ID NO: 184 - protein, 107 aa, Homo sapiens
DIQMTQSPSSLSASVGDRVTITCRASQSVSSAVAWYQQKPGKAPKLLIYSASSLYSGVPSRFSGSRSGTDFTLTISSLQPEDFATYYCQQSRQRPLTFGQGTKVEIK

SEQ ID NO: 185 - protein, 107 aa, Homo sapiens
DIQMTQSPSSLSASVGDRVTITCRASQSVSSAVAWYQQKPGKAPKLLIYSASSLYSGVPSRFSGSRSGTDFTLTISSLQPEDFATYYCQQSGLMPLTFGQGTKVEIK

SEQ ID NO: 186 - protein, 107 aa, Homo sapiens
DIQMTQSPSSLSASVGDRVTITCRASQSVSSAVAWYQQKPGKAPKLLIYSASSLYSGVPSRFSGSRSGTDFTLTISSLQPEDFATYYCQQSGHRPITFGQGTKVEIK

SEQ ID NO: 187 - protein, 107 aa, Homo sapiens
DIQMTQSPSSLSASVGDRVTITCRASQSVSSAVAWYQQKPGKAPKLLIYSASSLYSGVPSRFSGSRSGTDFTLTISSLQPEDFATYYCQQSSQRPATFGQGTKVEIK

SEQ ID NO: 188 - protein, 107 aa, Homo sapiens
DIQMTQSPSSLSASVGDRVTITCRASQSVSSAVAWYQQKPGKAPKLLIYSASSLYSGVPSRFSGSRSGTDFTLTISSLQPEDFATYYCQQSGRQPLTFGQGTKVEIK

SEQ ID NO: 189 - protein, 107 aa, Homo sapiens
DIQMTQSPSSLSASVGDRVTITCRASQSVSSAVAWYQQKPGKAPKLLIYSASSLYSGVPSRFSGSRSGTDFTLTISSLQPEDFATYYCQQFMRPAFTFGQGTKVEIK

SEQ ID NO: 190 - protein, 107 aa, Homo sapiens
DIQMTQSPSSLSASVGDRVTITCRASQSVSSAVAWYQQKPGKAPKLLIYSASSLYSGVPSRFSGSRSGTDFTLTISSLQPEDFATYYCQQSGQPSMTFGQGTKVEIK

SEQ ID NO: 191 - protein, 107 aa, Homo sapiens
DIQMTQSPSSLSASVGDRVTITCRASQSVSSAVAWYQQKPGKAPKLLIYSASSLYSGVPSRFSGSRSGTDFTLTISSLQPEDFATYYCQQSVLRPFTFGQGTKVEIK

SEQ ID NO: 192 - protein, 217 aa, Homo sapiens
MASDIQMTQSPSSLSASVGDRVTITCRASQSVSSAVAWYQQKPGKAPKLLIYSASSLYSGVPSRFSGSRSGTDFTLTISSLQPEDFATYYCQQSGQRPITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 193 - protein, 214 aa, Homo sapiens
DIQMTQSPSSLSASVGDRVTITCRASQSVSSAVAWYQQKPGKAPKLLIYSASSLYSGVPSRFSGSRSGTDFTLTISSLQPEDFATYYCQQSGQRPITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 194 - protein, 214 aa, Homo sapiens; identical to SEQ ID NO: 193

SEQ ID NO: 195 - protein, 214 aa, Homo sapiens; identical to SEQ ID NO: 193

SEQ ID NO: 196 - protein, 214 aa, Homo sapiens; identical to SEQ ID NO: 193

SEQ ID NO: 197 - protein, 214 aa, Homo sapiens; identical to SEQ ID NO: 193

SEQ ID NO: 198 - protein, 214 aa, Homo sapiens; identical to SEQ ID NO: 193

SEQ ID NO: 199 - protein, 217 aa, Homo sapiens
MASDIQMTQSPSSLSASVGDRVTITCRASQSVSSAVAWYQQKPGKAPKLLIYSASSLYSGVPSRFSGSRSGTDFTLTISSLQPEDFATYYCQQSSSSLITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 200 - protein, 214 aa, Homo sapiens
DIQMTQSPSSLSASVGDRVTITCRASQSVSSAVAWYQQKPGKAPKLLIYSASSLYSGVPSRFSGSRSGTDFTLTISSLQPEDFATYYCQQSSSSLITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 201 - protein, 214 aa, Homo sapiens; identical to SEQ ID NO: 200

SEQ ID NO: 202 - protein, 211 aa, Homo sapiens
MTQSPSSLSASVGDRVTITCRASQSVSSAVAWYQQKPGKAPKLLIYSASSLYSGVPSRFSGSRSGTDFTLTISSLQPEDFATYYCQQSSSSLITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 203 - protein, 211 aa, Homo sapiens
MTQSPSSLSASVGDRVTITCRASQSVSSAVAWYQQKPGKAPKLLIYSASSLYSGVPSRFSGSRSGTDFTLTISSLQPEDFATYYCQQSGQRPITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 204 - protein, 211 aa, Homo sapiens; identical to SEQ ID NO: 203

SEQ ID NO: 205 - protein, 211 aa, Homo sapiens; identical to SEQ ID NO: 203

SEQ ID NO: 206 - protein, 211 aa, Homo sapiens; identical to SEQ ID NO: 203

SEQ ID NO: 207 - protein, 211 aa, Homo sapiens; identical to SEQ ID NO: 203

SEQ ID NO: 208 - protein, 211 aa, Homo sapiens
MTQSPSSLSASVGDRVTITCRASQSVSSAVAWYQQKPGKAPKLLIYSASSLYSGVPSRFSGSRSGTDFTLTISSLQPEDFATYYCQQSGERPITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 209 - protein, 211 aa, Homo sapiens
MTQSPSSLSASVGDRVTITCRASQSVSSAVAWYQQKPGKAPKLLIYSASSLYSGVPSRFSGSRSGTDFTLTISSLQPEDFATYYCQQSGNVPATFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 210 - protein, 211 aa, Homo sapiens
MTQSPSSLSASVGDRVTITCRASQSVSSAVAWYQQKPGKAPKLLIYSASSLYSGVPSRFSGSRSGTDFTLTISSLQPEDFATYYCQQSGKRPMTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 211 - protein, 211 aa, Homo sapiens
MTQSPSSLSASVGDRVTITCRASQSVSSAVAWYQQKPGKAPKLLIYSASSLYSGVPSRFSGSRSGTDFTLTISSLQPEDFATYYCQQTGQRPMTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 212 - protein, 211 aa, Homo sapiens
MTQSPSSLSASVGDRVTITCRASQSVSSAVAWYQQKPGKAPKLLIYSASSLYSGVPSRFSGSRSGTDFTLTISSLQPEDFATYYCQQSMQWPKTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 213 - protein, 211 aa, Homo sapiens
MTQSPSSLSASVGDRVTITCRASQSVSSAVAWYQQKPGKAPKLLIYSASSLYSGVPSRFSGSRSGTDFTLTISSLQPEDFATYYCQQSLQRPMTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 214 - protein, 211 aa, Homo sapiens
MTQSPSSLSASVGDRVTITCRASQSVSSAVAWYQQKPGKAPKLLIYSASSLYSGVPSRFSGSRSGTDFTLTISSLQPEDFATYYCQQVELVPMTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 215 - protein, 211 aa, Homo sapiens
MTQSPSSLSASVGDRVTITCRASQSVSSAVAWYQQKPGKAPKLLIYSASSLYSGVPSRFSGSRSGTDFTLTISSLQPEDFATYYCQQAGKRPLTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 216 - protein, 211 aa, Homo sapiens
MTQSPSSLSASVGDRVTITCRASQSVSSAVAWYQQKPGKAPKLLIYSASSLYSGVPSRFSGSRSGTDFTLTISSLQPEDFATYYCQQSVDLTATFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 217 - protein, 211 aa, Homo sapiens
MTQSPSSLSASVGDRVTITCRASQSVSSAVAWYQQKPGKAPKLLIYSASSLYSGVPSRFSGSRSGTDFTLTISSLQPEDFATYYCQQLGVRAATFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 218 - protein, 211 aa, Homo sapiens
MTQSPSSLSASVGDRVTITCRASQSVSSAVAWYQQKPGKAPKLLIYSASSLYSGVPSRFSGSRSGTDFTLTISSLQPEDFATYYCQQSGRKPVTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 219 - protein, 211 aa, Homo sapiens
MTQSPSSLSASVGDRVTITCRASQSVSSAVAWYQQKPGKAPKLLIYSASSLYSGVPSRFSGSRSGTDFTLTISSLQPEDFATYYCQQTAQSPLTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 220 - protein, 211 aa, Homo sapiens
MTQSPSSLSASVGDRVTITCRASQSVSSAVAWYQQKPGKAPKLLIYSASSLYSGVPSRFSGSRSGTDFTLTISSLQPEDFATYYCQQSRQRPLTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 221 - protein, 211 aa, Homo sapiens
MTQSPSSLSASVGDRVTITCRASQSVSSAVAWYQQKPGKAPKLLIYSASSLYSGVPSRFSGSRSGTDFTLTISSLQPEDFATYYCQQSGLMPLTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 222 - protein, 211 aa, Homo sapiens
MTQSPSSLSASVGDRVTITCRASQSVSSAVAWYQQKPGKAPKLLIYSASSLYSGVPSRFSGSRSGTDFTLTISSLQPEDFATYYCQQSGHRPITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 223 - protein, 211 aa, Homo sapiens
MTQSPSSLSASVGDRVTITCRASQSVSSAVAWYQQKPGKAPKLLIYSASSLYSGVPSRFSGSRSGTDFTLTISSLQPEDFATYYCQQSSQRPATFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 224 - protein, 211 aa, Homo sapiens
MTQSPSSLSASVGDRVTITCRASQSVSSAVAWYQQKPGKAPKLLIYSASSLYSGVPSRFSGSRSGTDFTLTISSLQPEDFATYYCQQSGRQPLTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 225 - protein, 211 aa, Homo sapiens
MTQSPSSLSASVGDRVTITCRASQSVSSAVAWYQQKPGKAPKLLIYSASSLYSGVPSRFSGSRSGTDFTLTISSLQPEDFATYYCQQFMRPAFTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 226 - protein, 211 aa, Homo sapiens
MTQSPSSLSASVGDRVTITCRASQSVSSAVAWYQQKPGKAPKLLIYSASSLYSGVPSRFSGSRSGTDFTLTISSLQPEDFATYYCQQSGQPSMTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 227 - protein, 211 aa, Homo sapiens
MTQSPSSLSASVGDRVTITCRASQSVSSAVAWYQQKPGKAPKLLIYSASSLYSGVPSRFSGSRSGTDFTLTISSLQPEDFATYYCQQSVLRPFTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 228 - protein, 210 aa, Homo sapiens
MTQSPSSLSASVGDRVTITCRASQSVSSAVAWYQQKPGKAPKLLIYSASSLYSGVPSRFSGSRSGTDFTLTISSLQPEDFATYYCQQSSSSLITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGE

SEQ ID NO: 229 - protein, 116 aa, Homo sapiens
NIVMTQSPQSMSMSVGERVTLSCKASENVGAYVSWFQQKPDQSPKLLILAASNRYTGVPARFIGSGSATDFTLTISSVQAEDLADYHCGQSYTYPYTFGGGTKLEIKRADAAPTVS

SEQ ID NO: 230 - protein, 213 aa, Homo sapiens
IVMTQSPQSMSMSVGERVTLSCKASENVGAYVSWFQQKPDQSPKLLILAASNRYTGVPARFIGSGSATDFTLTISSVQAEDLADYHCGQSYTYPYTFGGGTKLEIKRADAAPTVSIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 231 - protein, 112 aa, Homo sapiens
DVLMTQTPLSLPVSLGDQASISCRSSQNIVHSYGNTYLEWYLQKPGQSPKLLIYKVSNRFSGVPDRFSGSGSGTDFTLKISRVEAEDLGVYYCFQGSHVPWTFGGGTKLEIK

SEQ ID NO: 232 - protein, 112 aa, Homo sapiens
DVVMTQTPLTLSVTIGQPASISCKSSQSLLYTNGKTYLIWLLQRPGQSPKRLIYLVSKLDSGVPDRFSGSGSGTDFTLKISRVEAEDLGVYYCLQSTHFPYTFGGGTKLEIK

SEQ ID NO: 233 - protein, 112 aa, Homo sapiens
DVVLTQTPLSLPVIIGDQASISCKSTKSLLNSAGFTYLGWYLQKPGQSPQLLIYLVSNRFSGVPDRFSGSGSGTDFTLKISRVEAEDLGVYFCFQSSYLPLTFGSGTKLEIK

SEQ ID NO: 234 - protein, 106 aa, Homo sapiens; identical to SEQ ID NO: 102

SEQ ID NO: 235 - protein, 106 aa, Homo sapiens
EIVLTQSPATLSLSPGERATLSCRASQSVGSYLAWYQQRPGQAPRLLIYDASNRATGIPARFSGSGSGTDFTLTISSLEPEDFAVYYCQQRAQEFTFGQGTKVEIK

SEQ ID NO: 236 - protein, 106 aa, Homo sapiens
EIVMTQSPATLSLSPGERATLSCRASQSVGSYLAWYQQKPGQAPRLLIYDASNRATGIPARFSGSGSGTDFTLTISSLQPEDFAVYYCQQRAQEFTFGQGTKVEIK

SEQ ID NO: 237 - protein, 106 aa, Homo sapiens; identical to SEQ ID NO: 236

SEQ ID NO: 238 - protein, 106 aa, Homo sapiens; identical to SEQ ID NO: 102

SEQ ID NO: 239 - protein, 106 aa, Homo sapiens; identical to SEQ ID NO: 102

SEQ ID NO: 240 - protein, 108 aa, Homo sapiens
EIVMTQSPDTLSLSPGERATLSCRASQKIYYSYLAWYQQKPGQAPRLLISGASTRASDISDRFSGGGSGTDFTLTINSLESEDAAVYYCQQYDSLPVTFGRGTKLEIK

SEQ ID NO: 241 - protein, 109 aa, Homo sapiens
SYELTQPPSVSVSPGQTARITCSGDALPKKYAFWYQQKSGQAPVLVIYEDSKRPSGIPEKFSGSSSGTMATLTISGAQVEDEADYYCYSTDSSDNPRGVFGGGTKLTVL

SEQ ID NO: 242 - protein, 107 aa, Homo sapiens
DIQMTQSPSSLSASVGDRVTITCRASQGISIYLAWFQQKPGKVPKLLIYAASTLQSGVPSRFSGSGSGTDFTLTISSLQPEDVATYYCQKYNSAPFTFGPGTKVEIK

SEQ ID NO: 243 - protein, 107 aa, Homo sapiens
EIVMTQSPATLSVSPGERATLSCRASQSVSSNLAWYQQKPGQAPRLLIYGASTRATGIPARFSGSGSGTEFTLTISSLQSEDFAVYYCQQYNNWPLTFGGGTKVEIK

SEQ ID NO: 244 - protein, 107 aa, Homo sapiens
DIQMTQSPSSLSASVGDRVTITCRASQDISIYLAWFQQKPGKVPKVLIYAASTLQSGVPSRFSGSGTGTDFTLTISSLQPEDVATYFCQKYNSAPFTFGPGTKLEIK

SEQ ID NO: 245 - protein, 107 aa, Homo sapiens; identical to SEQ ID NO: 19

SEQ ID NO: 246 - protein, 107 aa, Homo sapiens
DIQLTQSPSFLSASVGDRVTITCRASQGISSYLAWYQQKPGKAPKLLIYAASTLQSGVPSRFSGSGSGTEFTLTISSLQPEDFATYYCQQLNSYPRTFGQGTKVEIK

SEQ ID NO: 247 - protein, 104 aa, Homo sapiens
DIQLTQSPSFLSASVGDRVTITCRASQDISNYLAWYQQKPGKAPKLLISASTLQSGVPSRFSGSGSGTEFILTISSLQPEDFATYYCQHLNSYPFGQGTKVEIK

SEQ ID NO: 248 - protein, 112 aa, Homo sapiens
DIVMTQSPLSLPVTPGEPASISCRSSRSLLHSSGYNFLDWFLQKPGQSPQLLIFLGSDRASGVPDRFSGSGSGTDFTLKISRVETEDVGVYYCMQALQTPITFGQGTRLEIK

SEQ ID NO: 249 - protein, 112 aa, Homo sapiens
DIVMTQSPLSLPVTPGEPASISCRSSQSLLHSSGHNFLDWYLQKPGQSPQLLIYLGSNRASGVPDRFSGSGSGTDFTLKISRVEAEDVGVYYCMQALQTPPTFGQGTKLEIK

SEQ ID NO: 250 - protein, 107 aa, Homo sapiens; identical to SEQ ID NO: 243

SEQ ID NO: 251 - protein, 107 aa, Homo sapiens
EIVMTQSPATLSVSPGERATLSCRASQSVSSNLAWYQQKPGQAPRLLIYGASTRATGIPARFSGSGSGTEFTLTISSLQSEDFAVYYCQQYNNWPLTFGGGTKLEIK

SEQ ID NO: 252 - protein, 107 aa, Homo sapiens
DIQMTQSPSTLSASVGDRVTITCRASQSISTWLAWYQQKPGKAPKFLIYKASSLESGVPSRFSGSGSGTEFTLTISSLQPDDFATYYCQQYSGYSLTFGGGTKVEIK

SEQ ID NO: 253 - protein, 108 aa, Homo sapiens
EIVLTQSPATLSLSPGERATLSCRASQTVPNYLAWYQQKPGQAPRLLIYDASNRATDIPARFSGSGSGTDFTLTISSLEPEDFAVYYCQQRANWPPITFGQGTRLEIK

SEQ ID NO: 254 - protein, 107 aa, Homo sapiens
EIVLTQSPATLSLSPGERATLSCRASQSVRRYLAWYQQKPGQAPRLLIYDASNRATGIPARFSGSGSGTDFTLTISSLEPEDFAVYYCQQRNNWPLTFGGGTKLEIK

SEQ ID NO: 255 - protein, 112 aa, Homo sapiens
DIVMTQSPLSLPVTPGEPASISCRSSQSLLHSNGYNYLDWYLQKPGQSPQILIYLGSNRASGVPDRFSGSGSGTDFTLKISRVEAEDVGVYYCMQALQTPWTFGQGTKLEIK

SEQ ID NO: 256 - protein, 107 aa, Homo sapiens
DIQLTQSPSFLSASVGDRVTITCRASQGISNYLAWYQQKPGKVPKLLIYAVSTLQSGVPSRFSGSGSGTEFTLTISSLQPEDFATYYCQQLNSYPFTFGGGTKLEIK

SEQ ID NO: 257 - protein, 107 aa, Homo sapiens; identical to SEQ ID NO: 5

SEQ ID NO: 258 - protein, 107 aa, Homo sapiens
EIVMTQSPATLSVSPGERATLSCRASQSVSSNLAWYQQKPGQAPRLLIYGASTRATGIPARFSGSGSGTEFTLTFSSLQSEDFVVYYCQQYNNWPLTFGQGTRLEIK

SEQ ID NO: 259 - protein, 107 aa, Homo sapiens
DILMTQSPSSLSASVGDRVTITCRASQGINYYLAWYQQKPGKVPKLLIYTASTLQSGVPSRFSGSGSGTDFTLTISSLQPEDVATYFCQKYNSAPFTFGPGTKLEIK

SEQ ID NO: 260 - protein, 107 aa, Homo sapiens
EIVMTQSPATLSVSPGERGTLSCRASQSVSSNLAWYQQKPGQAPRLLIYGASIRATGIPARFSGSGSGTEFILTINSLQSEDFAVYYCQQYNNWPLTVGGGTKVEIK

SEQ ID NO: 261 - protein, 107 aa, Homo sapiens
DIQMTQSPSSLSASVGDRVTITCRASQGISIYLAWFQQKPGKVPKLLIYAASTLQSGVPSRFSGSGSGTDFTLTISSLQPEDFATYYCQKYNSAPFTFGPGTKLEIK

SEQ ID NO: 262 - protein, 107 aa, Homo sapiens
DIQMTQSPSSLSASVGDRVTITCRASQGITIYLAWFQQKPGKVPKLLIYAASTLQSGVPSRFSGSGSGTDFTLTISSLQPEDVATYYCQKYNSAPFTFGPGTKLEIK

SEQ ID NO: 263 - protein, 107 aa, Homo sapiens
DIQMTQSPSSLSASVGDRVTITCRASQGISIYLAWFQQKPGKVPKLLIYAASTLQSGVPSRFSGSGSGTDFTLTISSLQPEDVATYYCQKYNSAPFTFGPGTKLEIK

SEQ ID NO: 264 - protein, 107 aa, Homo sapiens
DIQMTQSPSSLSASVGDRVTITCRASQDISIYLAWFQQRPGKVPKLLIYAASTLQSGVPSRFSGSGSGTDFTLTISSLQPEDVATYYCQKYNSDPFTFGPGTKVEIK

SEQ ID NO: 265 - protein, 107 aa, Homo sapiens
DIQMTQSPSSLSASVGDRVTITCRASQDISIYLAWFHQKPGKVPKLLIYAASTLQSGVPSRFSGSGSGTDFTLTISSLQPEDVAAYYCQKYNSAPFTFGPGTKLEIK

SEQ ID NO: 266 - protein, 107 aa, Homo sapiens
DIQMTQSPSSLSATVGDRVTITCRASQGISIYLAWFQQKPGKVPKNLIYAASTLQSGVPSRFSGSGSGTDFTLTISSLQPEDFATYYCQKYNSAPFTFGPGTKLEIK

SEQ ID NO: 267 - protein, 107 aa, Homo sapiens
DIQMTQSPSSLSASVGDRVTITCRASQGISIYLAWYHQKPGKVPKLLIYAASTLQSGVPSRFSGSGSGTDFTLTISSLQPEDVATYYCQKYNSAPWTFGQGTKLEIK

SEQ ID NO: 268 - protein, 113 aa, Homo sapiens
DIVMTQSPLSLPVTPGEPASISCRSSQSLLHSNGYNYLDWYLQKSGQSPQLLIYLGSNRASGVPDRFSGSGSGTDFTLKISRVEAEDVGVYYCMQALLIPLYTFGQGTKLEIK

SEQ ID NO: 269 - protein, 112 aa, Homo sapiens
DIVMTQSPLSLPVTPGEPASISCRSSQSLLHSSGHNFLDWYLKKPGQSPQLLIYLGSNRGSGVPDRFSGSGSGTDFTLKISRVEAEDVGVYYCMQALQTPRTFGQGTKLEIK

SEQ ID NO: 270 - protein, 112 aa, Homo sapiens
DIVMTQSPLSLPVTPGEPASISCRSSQSLLHSTGYNFLDWYLQKPGQSPQLLIFLGSNRASGVPDRFSGSGSGTDFTLKISRVEAEDVGIYYCMQALQTPLTFGGGTKVEIK

SEQ ID NO: 271 - protein, 107 aa, Homo sapiens
EIVMTQSPATLSVSPGERATLSCRASQSVSNNLAWYQQKPGQAPRLLIYGVSTRATGIPARFSGSGSGTEFTLIISSLQSEDFAGYYCQQYNDWPLTFGGGTKLEIK

SEQ ID NO: 272 - protein, 106 aa, Homo sapiens
DIQMTQSPSTLSASVGDRVTITCRASQSISSWLAWYQQKPGKAPNLLIYKASSLESGVPSRFSGSGSGTEFTLTISSLQPDDFATYYCQQYNTYYTFGQGTKVEIK

SEQ ID NO: 273 - protein, 112 aa, Homo sapiens
DIVMTQSPLSLPVTPGEPASISCRSSQSLLHSSGYNYLDWYLQKPGQSPQLLIYLGSDRASGVPDRFRGSGSGTDFTLKISRVEAEDVGIYYCMQALQIPYTFGQGTKLEIK

SEQ ID NO: 274 - protein, 107 aa, Homo sapiens
DIQMTQSPSSLSASVGDRVTITCRASQSISSYLNWFQQKPGKAPKLLIYAASSLQSGVPSRFSGSGSGTDFTLTISSLQPEDFATYYCQQSYTMPYTFGQGTKLEIK

SEQ ID NO: 275 - protein, 107 aa, Homo sapiens
EIVLTQSPATLSLSPGERATLSCRASQSVTNYLAWFQQKPGQAPRLLIYDAFNRATGIPARFSGSGSGTDFTLTISSLEPEDFAIYYCQQRSYWPLTFGGGTKLEIK

SEQ ID NO: 276 - protein, 107 aa, Homo sapiens
DIQLTQSPSFLSASVGDRVTITCRASQGISSYLAWYQQKPGKAPKLLIYAASTLQSGVPSRFSGSGSGTEFTLTISSLQPEDFATYYCQQLNSYPYTFGQGTKVEIK

SEQ ID NO: 277 - protein, 106 aa, Homo sapiens
DIQMTQSPSTLSASVGDRVTITCRASQSISSWLAWYQQKPGKAPKLLIYKASSLESGVPSRFSGSGSGTEFTLTISSLQPDDFATYYCQQYYSYITFGQGTRLEIK

SEQ ID NO: 278 - protein, 107 aa, Homo sapiens
DIQLTQSPSFLSASVGDRVTITCRASQGISSYLAWYQQKPGKAPKLLIYAASTLQSGVPSRFSGSGSGTEFTLTISSLQPEDFATYYCQQLNSYPYTFGQGTKLEIK

SEQ ID NO: 279 - protein, 106 aa, Homo sapiens
EIVLTQSPATLSLSPGERATLSCRASQSVRSYLAWYQQKPGQAPRLLIYDASKRATGIPARFSGSGSGTDFTLTISSLEPEDFAVYYCQQRNSWPTFGGGTKVEIK

SEQ ID NO: 280 - protein, 108 aa, Homo sapiens
DIQLTQSPSFLSASVGDRVTITCRASQGISSYLAWYQQKPGKAPKLLIYAASTLQSGVPSRFSGSGSGTEFTLTISSLQPEDFAIYYCQQLNSYPLYTFGQGTKLEIK

SEQ ID NO: 281 - protein, 108 aa, Homo sapiens
DIQLTQSPSFLSASVGDRVTITCRASQGISTYLAWYQQKPGKAPKLLIYAASTLQSGVPSRFSGSGSGTEFTLTISSLQPEDFATYYCQQLNSYPLYTFGQGTKVEIK

SEQ ID NO: 282 - protein, 97 aa, Homo sapiens
DIQMTQTTSSLSASLGDRVTISCRASQDISNYLNWYQQKPDGTVKLLIYYTSRLHSGVPSRFSGSGSGTDYSLTISNLEQEDIATYFCQQGNTLPWT

SEQ ID NO: 283 - protein, 214 aa, Homo sapiens
DIQMTQTTSSLSASLGDRVTISCRASQDISNYLNWYQQKPDGTVKLLIYYTSRLHSGVPSRFSGSGSGTDYSLTISNLEQEDIATYFCQQGNTLPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 284 - protein, 120 aa, Homo sapiens; identical to SEQ ID NO: 147

SEQ ID NO: 285 - protein, 120 aa, Homo sapiens; identical to SEQ ID NO: 148

SEQ ID NO: 286 - protein, 120 aa, Homo sapiens; identical to SEQ ID NO: 149

SEQ ID NO: 287 - protein, 120 aa, Homo sapiens; identical to SEQ ID NO: 150

SEQ ID NO: 288 - protein, 120 aa, Homo sapiens; identical to SEQ ID NO: 151

SEQ ID NO: 289 - protein, 120 aa, Homo sapiens; identical to SEQ ID NO: 152

SEQ ID NO: 290 - protein, 120 aa, Homo sapiens; identical to SEQ ID NO: 153

SEQ ID NO: 291 - protein, 120 aa, Homo sapiens; identical to SEQ ID NO: 154

SEQ ID NO: 292 - protein, 120 aa, Homo sapiens; identical to SEQ ID NO: 155

SEQ ID NO: 293 - protein, 120 aa, Homo sapiens; identical to SEQ ID NO: 156

SEQ ID NO: 294 - protein, 120 aa, Homo sapiens; identical to SEQ ID NO: 157

SEQ ID NO: 295 - protein, 112 aa, Homo sapiens
DVLMTQTPLSLPVSLGDQASISCRSSQSIVHSDGNTYLEWYLQKPGQSPKLLIYKVSNRFSGVPDRFSGSGSGTDFTLKISRVEAEDLGVYYCFQGSHVPYTFGGGTKLEIK

(19) United States
(12) Patent Application Publication
(10) Pub. No.: US 2026/0263614 A1
(43) Pub. Date: Sep. 10, 2026

(54) ANTIBODY DRUG CONJUGATES THAT BIND CDCP1 AND USES THEREOF

(71) Applicant: Pheon Therapeutics Ltd, Hertfordshire (GB)

(72) Inventors: Paul Joseph Mark Jackson, Hertfordshire (GB); George Procopiou, Hertfordshire (GB); David Edwin Thurston, Hertfordshire (GB); Leigh Zawel, Hertfordshire (GB)

(21) Appl. No.: 18/994,498

(22) PCT Filed: Jul. 15, 2023
(86) PCT No.: PCT/IB2023/057250

§ 371 Date: Jan. 14, 2025

Related U.S. Application Data
(60) Provisional application No. 63/389,743, filed on Jul. 15, 2022.
(60) Provisional application No. 63/400,703, filed on Aug. 24, 2022.
(60) Provisional application No. 63/489,473, filed on Mar. 10, 2023.
(60) Provisional application No. 63/489,474, filed on Mar. 10, 2023.

(51) Int. Cl. A61K 47/68; A61P 35/00; C07K 16/28

(52) U.S. Cl. CPC A61K 47/68037; A61K 47/6849; A61K 47/6889; A61P 35/00; C07K 16/2896

(57) ABSTRACT

The present disclosure provides antibody-drug conjugates (ADCs), linkers, and compounds thereof useful for treating cancer.

### CROSS-REFERENCE TO RELATED APPLICATIONS

This application claims priority to U.S. Provisional Application Nos. 63/389,743, filed Jul. 15, 2022, 63/400,703, filed Aug. 24, 2022, 63/489,473, filed Mar. 10, 2023, and 63/489,474, filed Mar. 10, 2023, all of which are incorporated by reference herein in their entireties.

### FIELD

The disclosure generally relates to antibody-drug conjugates and methods for treating cancer and other diseases, including CUB domain-containing protein 1 (CDCP1)-targeted therapies.

### BACKGROUND

While numerous chemotherapeutic agents have been developed, many often demonstrate unacceptable toxicity and or lack of specificity for cancer cells over non-cancerous tissues. To avoid the non-specific cytotoxic effects of chemotherapeutic agents, targeted antibody therapy has revolutionized cancer treatment with several monoclonal antibodies demonstrating clinical potential. Because antibodies against tumor-specific antigens often lack therapeutic activities, they have been conjugated to cytotoxic agents in order to combine the effectiveness of chemotherapy with the targeting of antibodies. In principle, selective delivery of cytotoxic agents to specific tumor tissues by antibody binding should reduce the systemic toxicity of traditional small-molecule chemotherapeutics.

Since a successful antibody drug conjugate (ADC) approach must successfully bind to a target antigen in order to deliver a toxic payload to a target cell without significant binding to non-target cells, it is crucial that the ADC be able to deliver a toxic payload to a target cell, be internalized thereby, and then release the payload once inside the appropriate compartment within the cell.

CDCP1 is widely expressed in human epithelial tissues. CDCP1 functions in the tyrosine phosphorylation-dependent regulation of cellular events that are involved in tumor invasion and metastasis, but its phosphorylation is only observed in mitotically detached or shedding cells, consistent with its role in the negative regulation of cell adhesion. The phosphorylation of CDCP1 is seen in many cancers, including some pre-invasive cancers as well as in invasive tumors and in tumor metastases.

Despite a deepening understanding of tumor-specific proteins to target with ADC therapy, the need for specific CDCP1-targeted ADCs that can be used for therapeutic purposes in the treatment of cancer remains unmet in the art.

### SUMMARY

In aspects, the disclosure provides an antibody-drug conjugate having formula (I):

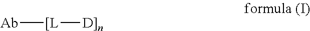

wherein in formula (I):
Ab comprises an antibody or binding fragment thereof, wherein the antibody or binding fragment thereof specifically binds CUB Domain-Containing Protein-1 (CDCP1);
L is a linker of the formula —R*-L₁-L_{A}-;
R* is succinimide;
L₁ is —[CH₂]₁₋₃—C(O)NH—;
L_{A} is —[CH₂CH₂O]ₚ—(CH₂)₁₋₅—C(O)—X_{AA}—, wherein p is an integer from 5 to 10, and X_{AA} is an amino acid sequence having 2 amino acid moieties;
D is selected from

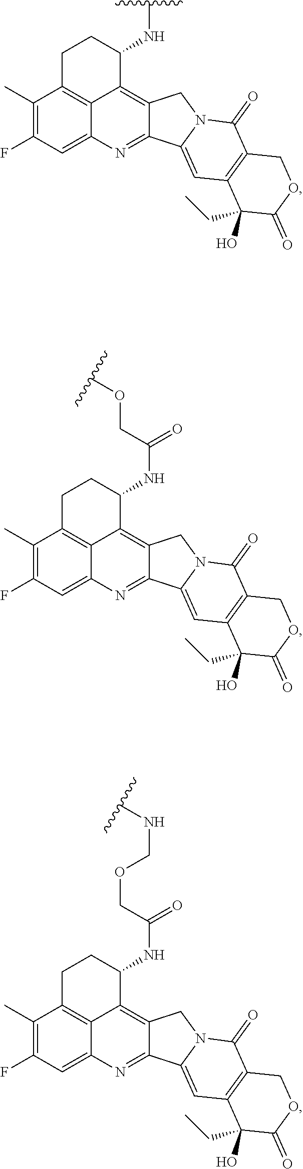 [SMILES: *N[C@H]1CCc2c(C)c(F)cc3nc4c(c1c23)Cn1c-4cc2c(c1=O)COC(=O)[C@]2(O)CC ; *OCC(=O)N[C@H]1CCc2c(C)c(F)cc3nc4c(c1c23)Cn1c-4cc2c(c1=O)COC(=O)[C@]2(O)CC ; *NCOCC(=O)N[C@H]1CCc2c(C)c(F)cc3nc4c(c1c23)Cn1c-4cc2c(c1=O)COC(=O)[C@]2(O)CC]

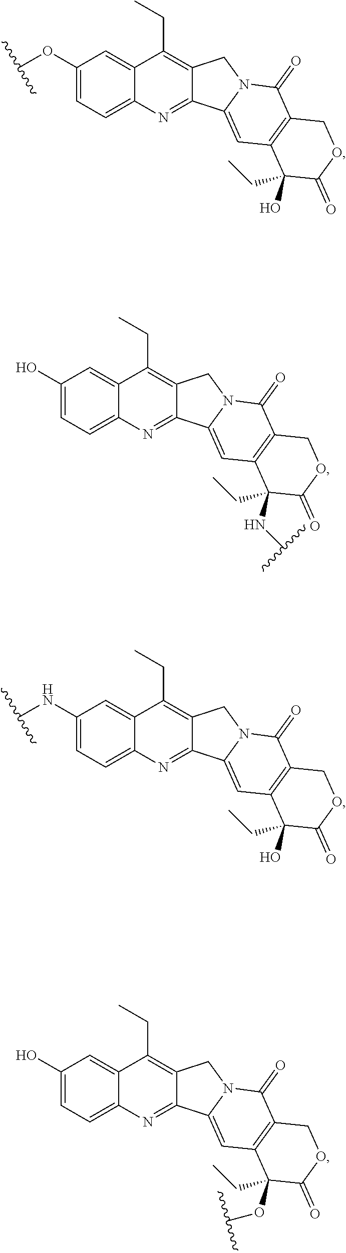 [SMILES: *Oc1ccc2nc3c(c(CC)c2c1)Cn1c-3cc2c(c1=O)COC(=O)[C@]2(O)CC ; *N[C@]1(CC)C(=O)OCc2c1cc1n(c2=O)Cc2c-1nc1ccc(O)cc1c2CC ; *Nc1ccc2nc3c(c(CC)c2c1)Cn1c-3cc2c(c1=O)COC(=O)[C@]2(O)CC ; *O[C@]1(CC)C(=O)OCc2c1cc1n(c2=O)Cc2c-1nc1ccc(O)cc1c2CC]

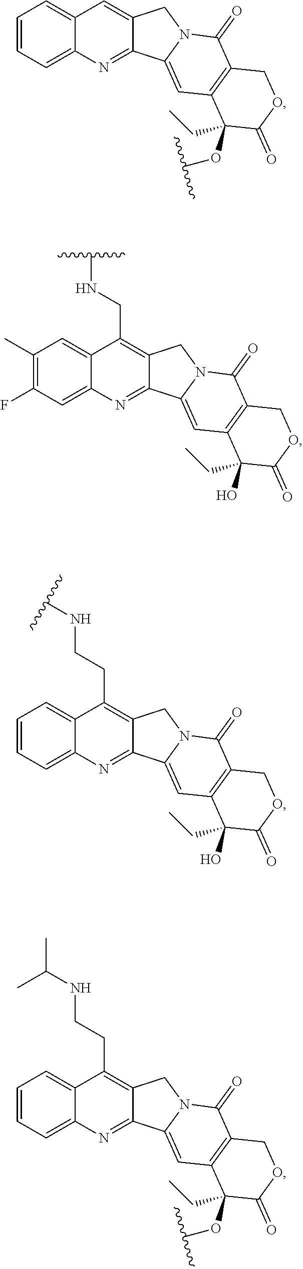 [SMILES: *O[C@]1(CC)C(=O)OCc2c1cc1n(c2=O)Cc2cc3ccccc3nc2-1 ; *NCc1c2c(nc3cc(F)c(C)cc13)-c1cc3c(c(=O)n1C2)COC(=O)[C@]3(O)CC ; *NCCc1c2c(nc3ccccc13)-c1cc3c(c(=O)n1C2)COC(=O)[C@]3(O)CC ; *O[C@]1(CC)C(=O)OCc2c1cc1n(c2=O)Cc2c-1nc1ccccc1c2CCNC(C)C]

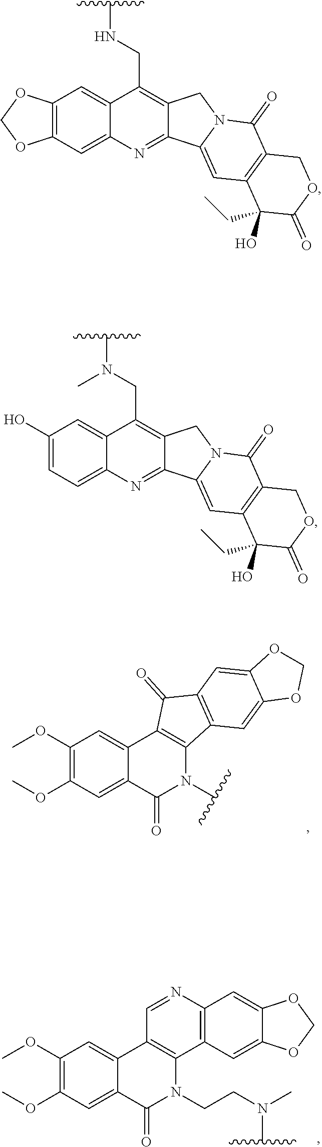 [SMILES: *NCc1c2c(nc3cc4c(cc13)OCO4)-c1cc3c(c(=O)n1C2)COC(=O)[C@]3(O)CC ; *N(C)Cc1c2c(nc3ccc(O)cc13)-c1cc3c(c(=O)n1C2)COC(=O)[C@]3(O)CC ; *n1c2c(c3cc(OC)c(OC)cc3c1=O)C(=O)c1cc3c(cc1-2)OCO3 ; *N(C)CCn1c(=O)c2cc(OC)c(OC)cc2c2cnc3cc4c(cc3c21)OCO4]

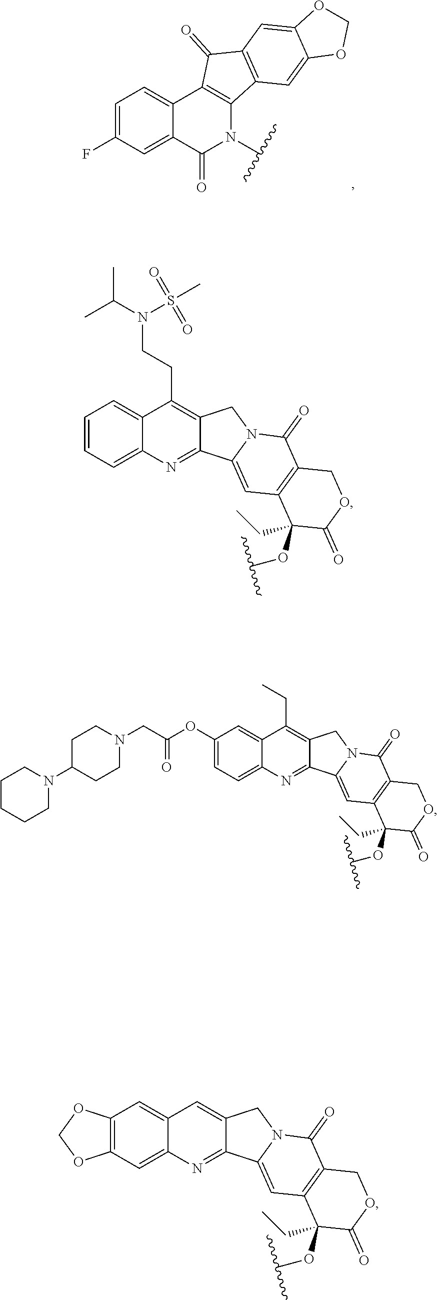 [SMILES: *n1c2c(c3ccc(F)cc3c1=O)C(=O)c1cc3c(cc1-2)OCO3 ; *O[C@]1(CC)C(=O)OCc2c1cc1n(c2=O)Cc2c-1nc1ccccc1c2CCN(C(C)C)S(C)(=O)=O ; *O[C@]1(CC)C(=O)OCc2c1cc1n(c2=O)Cc2c-1nc1ccc(OC(=O)CN3CCC(N4CCCCC4)CC3)cc1c2CC ; *O[C@]1(CC)C(=O)OCc2c1cc1n(c2=O)Cc2cc3cc4c(cc3nc2-1)OCO4]

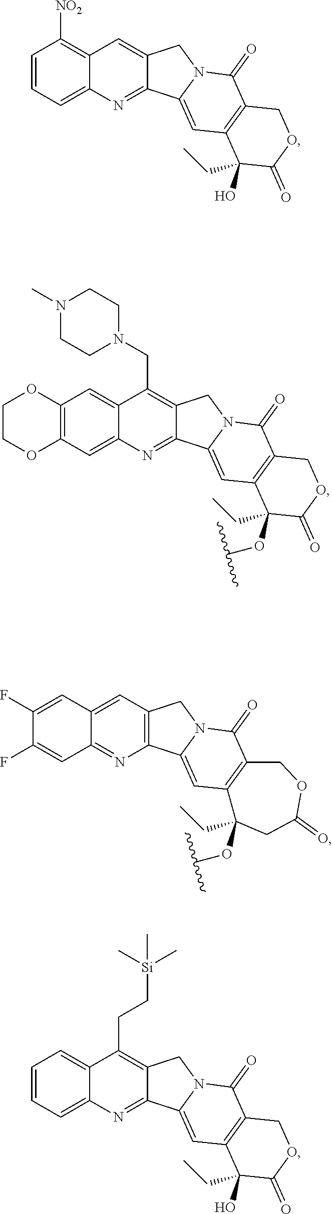 [SMILES: CC[C@@]1(O)C(=O)OCc2c1cc1n(c2=O)Cc2cc3c([N+](=O)[O-])cccc3nc2-1 ; *O[C@]1(CC)C(=O)OCc2c1cc1n(c2=O)Cc2c-1nc1cc3c(cc1c2CN1CCN(C)CC1)OCCO3 ; *O[C@]1(CC)CC(=O)OCc2c1cc1n(c2=O)Cc2cc3cc(F)c(F)cc3nc2-1 ; CC[C@@]1(O)C(=O)OCc2c1cc1n(c2=O)Cc2c-1nc1ccccc1c2CC[Si](C)(C)C]

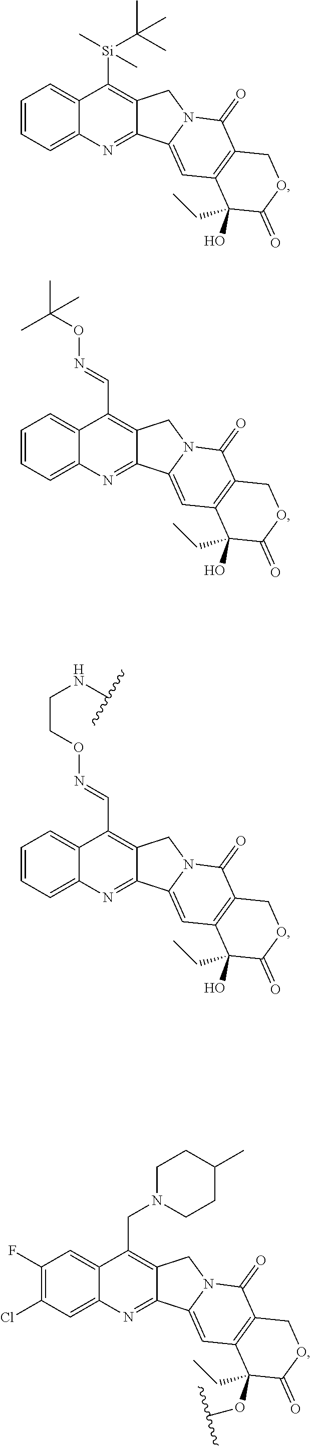 [SMILES: CC[C@@]1(O)C(=O)OCc2c1cc1n(c2=O)Cc2c-1nc1ccccc1c2[Si](C)(C)C(C)(C)C ; CC[C@@]1(O)C(=O)OCc2c1cc1n(c2=O)Cc2c-1nc1ccccc1c2/C=N/OC(C)(C)C ; *NCCO/N=C/c1c2c(nc3ccccc13)-c1cc3c(c(=O)n1C2)COC(=O)[C@]3(O)CC ; *O[C@]1(CC)C(=O)OCc2c1cc1n(c2=O)Cc2c-1nc1cc(Cl)c(F)cc1c2CN1CCC(C)CC1]

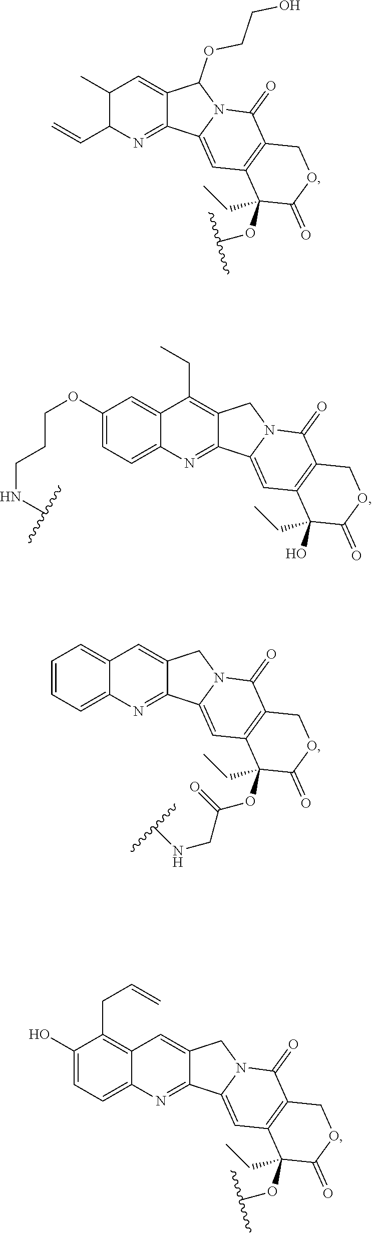 [SMILES: *O[C@]1(CC)C(=O)OCc2c1cc1n(c2=O)C(OCCO)C2=CC(C)C(C=C)N=C21 ; *NCCCOc1ccc2nc3c(c(CC)c2c1)Cn1c-3cc2c(c1=O)COC(=O)[C@]2(O)CC ; *NCC(=O)O[C@]1(CC)C(=O)OCc2c1cc1n(c2=O)Cc2cc3ccccc3nc2-1 ; *O[C@]1(CC)C(=O)OCc2c1cc1n(c2=O)Cc2cc3c(CC=C)c(O)ccc3nc2-1]

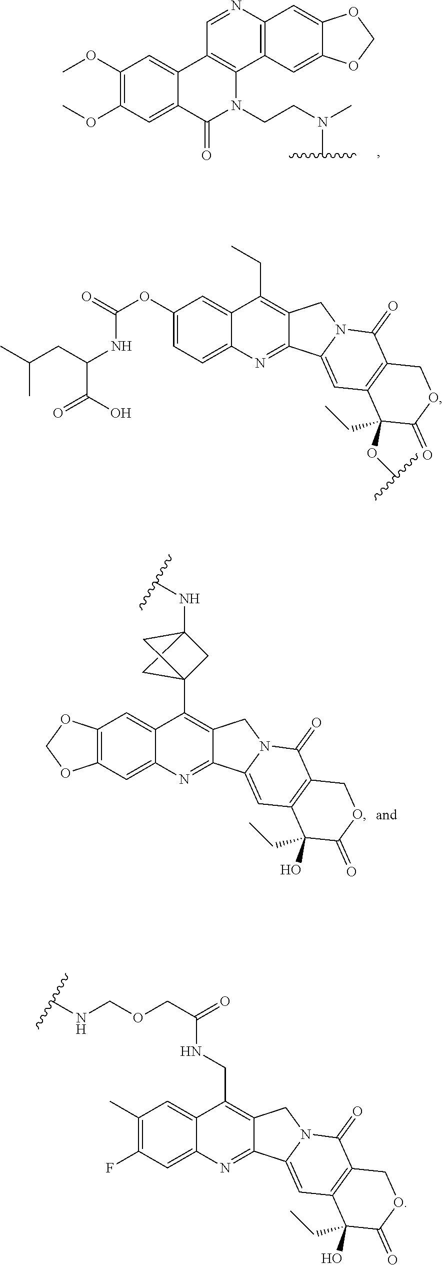 [SMILES: *N(C)CCn1c(=O)c2cc(OC)c(OC)cc2c2cnc3cc4c(cc3c21)OCO4 ; *O[C@]1(CC)C(=O)OCc2c1cc1n(c2=O)Cc2c-1nc1ccc(OC(=O)NC(CC(C)C)C(=O)O)cc1c2CC ; *NC12CC(c3c4c(nc5cc6c(cc35)OCO6)-c3cc5c(c(=O)n3C4)COC(=O)[C@]5(O)CC)(C1)C2 ; *NCOCC(=O)NCc1c2c(nc3cc(F)c(C)cc13)-c1cc3c(c(=O)n1C2)COC(=O)[C@]3(O)CC]

In some embodiments, L₁ is

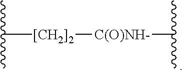

In some embodiments, p is 7 or 8. In some embodiments, p is 8. In some embodiments, X_{AA} is selected from Val-Ala, Tyr-Arg, Phe-Arg, Val-Gln, Val-Cit, Tyr-Met, Leu-Gln, Val-Arg, Met-Thr, Phe-Gln, Thr-Thr, Val-Thr, Ala-Ala, Val-Met, Leu-Met, Ala-Asn, D-Val-D-Gln, D-Ala-D-Ala, and Phe-Met. In some embodiments, X_{AA} is valine-alanine. In some embodiments, wherein L_{A} is —[CH₂CH₂O]ₚ—(CH₂)₁₋₃—C(O)—X_{AA}—. In some embodiments, L_{A} is —[CH₂CH₂O]ₚ—(CH₂)₂—C(O)—X_{AA}—. In some embodiments, the linker L has the formula:

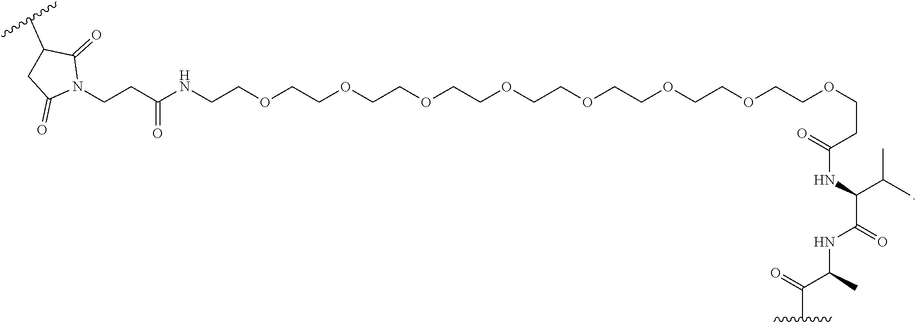 [SMILES: *C(=O)[C@H](C)NC(=O)[C@@H](NC(=O)CCOCCOCCOCCOCCOCCOCCOCCOCCNC(=O)CCN1C(=O)CC(*)C1=O)C(C)C]

In some embodiments, D is

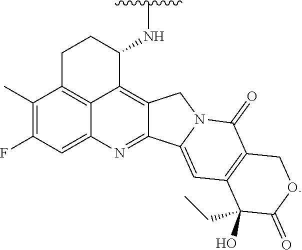 [SMILES: *N[C@H]1CCc2c(C)c(F)cc3nc4c(c1c23)Cn1c-4cc2c(c1=O)COC(=O)[C@]2(O)CC]

In some embodiments, L-D has the formula:

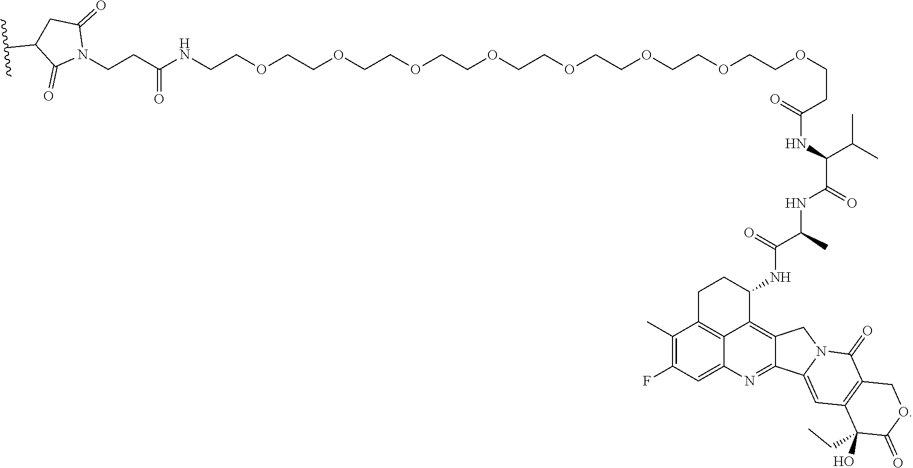 [SMILES: *C1CC(=O)N(CCC(=O)NCCOCCOCCOCCOCCOCCOCCOCCOCCC(=O)N[C@H](C(=O)N[C@@H](C)C(=O)N[C@H]2CCc3c(C)c(F)cc4nc5c(c2c34)Cn2c-5cc3c(c2=O)COC(=O)[C@]3(O)CC)C(C)C)C1=O]

In some embodiments, n is an integer from 4 to 8. In some embodiments, n is 4. In some embodiments, n is 8. In some embodiments, the antibody or binding fragment thereof comprises:
(i) a heavy chain variable region (VH) that comprises:
   (a) a VH complementarity determining region one (CDRH1) comprising and/or consisting of the amino acid sequence of SEQ ID NO: 2,
   (b) a VH complementarity determining region two (CDRH2) comprising and/or consisting of the amino acid sequence of SEQ ID NO: 3, and
   (c) a VH complementarity determining region three (CDRH3) comprising and/or consisting of the amino acid sequence of SEQ ID NO: 4,
and (ii) a light chain variable region (VL) that comprises:
   (a) a VL complementarity determining region one (CDRL1) comprising and/or consisting of the amino acid sequence of SEQ ID NO: 6,
   (b) a VL complementarity determining region two (CDRL2) comprising and/or consisting of the amino acid sequence of SEQ ID NO: 7, and
   (c) a VL complementarity determining region three (CDRL3) comprising and/or consisting of the amino acid sequence of SEQ ID NO:8.
      In some embodiments, the antibody or binding fragment thereof comprises:
(i) a heavy chain variable region (VH) that comprises:
   (a) a VH complementarity determining region one (CDRH1) comprising and/or consisting of the amino acid sequence of SEQ ID NO: 12,
   (b) a VH complementarity determining region two (CDRH2) comprising and/or consisting of the amino acid sequence of SEQ ID NO: 13, and
   (c) a VH complementarity determining region three (CDRH3) comprising and/or consisting of the amino acid sequence of SEQ ID NO: 14,
and (ii) a light chain variable region (VL) that comprises:
   (a) a VL complementarity determining region one (CDRL1) comprising and/or consisting of the amino acid sequence of SEQ ID NO: 6,
   (b) a VL complementarity determining region two (CDRL2) comprising and/or consisting of the amino acid sequence of SEQ ID NO: 7, and
   (c) a VL complementarity determining region three (CDRL3) comprising and/or consisting of the amino acid sequence of SEQ ID NO:8.
      In some embodiments, the antibody or binding fragment thereof comprises a VH that comprises an amino acid sequence at least 90%, at least 91%, at least 92%, at least 93%, at least 94%, at least 95%, at least 96%, at least 97%, at least 98%, at least 99%, or 100% identical to the amino acid sequence of SEQ ID NO: 1, and/or a VL that comprises an amino acid sequence at least 90%, at least 91%, at least 92%, at least 93%, at least 94%, at least 95%, at least 96%, at least 97%, at least 98%, at least 99%, or 100% identical to the amino acid sequence of SEQ ID NO: 5. In some embodiments, the antibody or binding fragment thereof comprises a VH that comprises the amino acid sequence of SEQ ID NO: 1 and/or a VL that comprises the amino acid sequence of SEQ ID NO: 5. In some embodiments, the antibody or binding fragment thereof comprises a heavy chain comprising an amino acid sequence at least 90%, at least 91%, at least 92%, at least 93%, at least 94%, at least 95%, at least 96%, at least 97%, at least 98%, at least 99%, or 100% identical to the amino acid sequence of SEQ ID NO: 1 and/or a light chain comprising an amino acid sequence at least 90%, at least 91%, at least 92%, at least 93%, at least 94%, at least 95%, at least 96%, at least 97%, at least 98%, at least 99%, or 100% identical to the amino acid sequence of SEQ ID NO: 5. In some embodiments, the antibody or binding fragment thereof comprises a heavy chain that comprises the amino acid sequence of SEQ ID NO: 1 and/or a light chain that comprises the amino acid sequence of SEQ ID NO: 5. In some embodiments, the antibody-drug conjugate has a drug-to-antibody ratio (DAR) ranging from about 1 to about 10, optionally wherein the DAR is about 1, about 2, about 3, about 4, about 5, about 6, about 7, about 8, about 9, or about 10, optionally DAR is about 4, optionally DAR is about 8.

In aspects, the disclosure provides a pharmaceutical composition comprising an antibody drug conjugate of formula (I); and a pharmaceutically acceptable carrier.

In aspects, the disclosure provides a method of treating a cancer comprising administering to a subject in need thereof a therapeutically effective amount of an antibody drug conjugate of formula (I), or a pharmaceutical composition of the disclosure. In some embodiments, less than about 50% of the antibody-drug conjugate is converted to a metabolite about 24 hours after administering the therapeutically effective amount of the antibody-drug conjugate to the subject. In some embodiments, about 50% of the antibody-drug conjugate is converted to a metabolite about 96 hours after administering the therapeutically effective amount of the antibody-drug conjugate to the subject. In some embodiments, the antibody-drug conjugate is converted to a metabolite of formula 300:

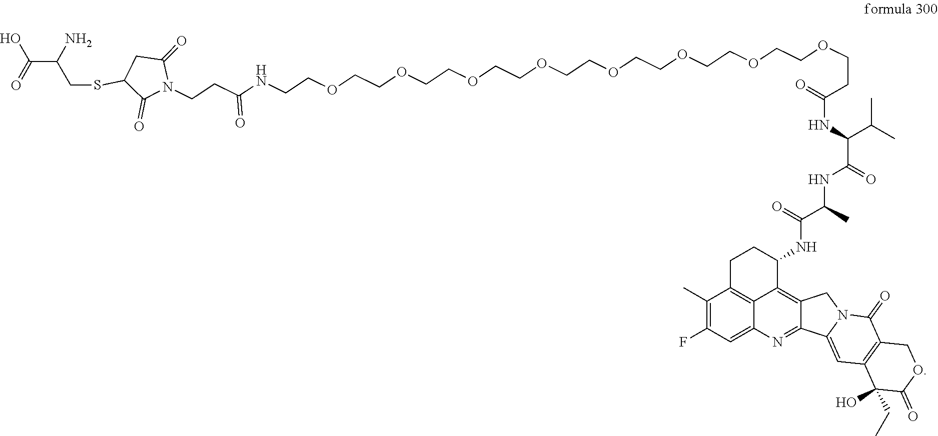 [SMILES: CC[C@@]1(O)C(=O)OCc2c1cc1n(c2=O)Cc2c-1nc1cc(F)c(C)c3c1c2[C@@H](NC(=O)[C@H](C)NC(=O)[C@@H](NC(=O)CCOCCOCCOCCOCCOCCOCCOCCOCCNC(=O)CCN1C(=O)CC(SCC(N)C(=O)O)C1=O)C(C)C)CC3]

In some embodiments, the antibody-drug conjugate is converted to a metabolite of formula 301:

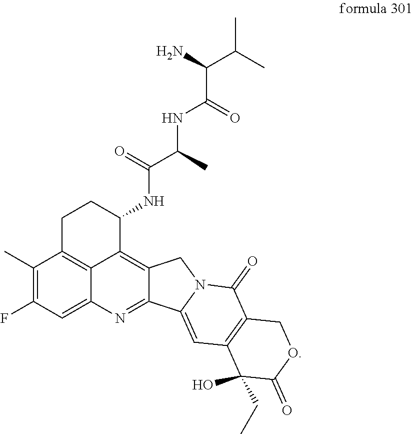 [SMILES: CC[C@@]1(O)C(=O)OCc2c1cc1n(c2=O)Cc2c-1nc1cc(F)c(C)c3c1c2[C@@H](NC(=O)[C@H](C)NC(=O)[C@@H](N)C(C)C)CC3]

In some embodiments, the antibody-drug conjugate is converted to a metabolite of formula 302:

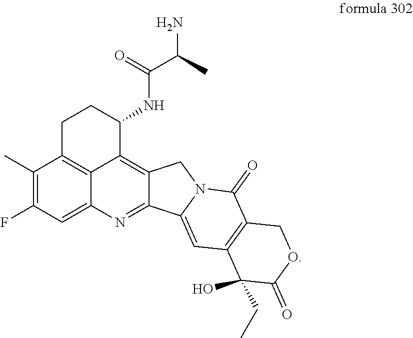 [SMILES: CC[C@@]1(O)C(=O)OCc2c1cc1n(c2=O)Cc2c-1nc1cc(F)c(C)c3c1c2[C@@H](NC(=O)[C@H](C)N)CC3]

In some embodiments, the antibody-drug conjugate is converted to a metabolite in vivo. In some embodiments, the antibody-drug conjugate is converted to a metabolite in vitro. In some embodiments, the cancer is selected from the group consisting of pancreatic cancer, breast cancer, prostate cancer, lymphoma, skin cancer, colon cancer, melanoma, malignant melanoma, ovarian cancer, brain cancer, primary brain carcinoma, head-neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, chronic lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, or retinoblastoma, and the like. In other embodiments, the cancer is acoustic neuroma, adenocarcinoma, angiosarcoma, astrocytoma, basal cell carcinoma, bile duct carcinoma, bladder carcinoma, brain cancer, breast cancer, triple-negative breast cancer (TNBC), bronchogenic carcinoma, cervical cancer, chordoma, choriocarcinoma, colon cancer, colorectal cancer, craniopharyngioma, cystadenocarcinoma, embryonal carcinoma, endotheliocarcinoma, ependymoma, epithelial carcinoma, esophageal cancer, Ewing's tumor, fibrosarcoma, gastric cancer, glioblastoma multiforme, glioma, head and neck cancer, hemangioblastoma, hepatoma, kidney cancer, leiomyosarcoma, liposarcoma, lung cancer, lymphangioendotheliosarcoma, lymphangiosarcoma, medullary carcinoma, medulloblastoma, melanoma, meningioma, mesothelioma, myxosarcoma, nasal cancer, neuroblastoma, oligodendroglioma, oral cancer, osteogenic sarcoma, ovarian cancer, pancreatic cancer, papillary adenocarcinoma, papillary carcinoma, pinealoma, prostate cancer, rabdomyosarcoma, rectal cancer, renal cell carcinoma, retinoblastoma, sarcoma, sebaceous gland carcinoma, seminoma, skin cancer, squamous cell carcinoma, stomach cancer, sweat gland carcinoma, synovioma, testicular cancer, small cell lung carcinoma, throat cancer, uterine cancer, Wilm's tumor, blood cancer, acute erythroleukemic leukemia, acute lymphoblastic B-cell leukemia, acute lymphoblastic T-cell leukemia, acute lymphoblastic leukemia, acute megakaryoblastic leukemia, acute monoblastic leukemia, acute myeloblastic leukemia, acute myelomonocytic leukemia, acute nonlymphocytic leukemia, acute promyelocytic leukemia, acute undifferentiated leukemia, chronic lymphocytic leukemia, chronic myelocytic leukemia, hairy cell leukemia, multiple myeloma, heavy chain disease, Hodgkin's disease, multiple myeloma, non-Hodgkin's lymphoma, polycythemia vera, or Waldenstrom's macroglobulinemia. In some embodiments, the cancer in triple-negative breast cancer (TNBC).

In aspects, the disclosure provides an antibody-drug conjugate having formula (I):

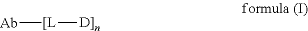

wherein in formula (I):
Ab comprises an antibody or antibody-binding fragment, wherein the antibody or binding fragment thereof specifically binds CUB Domain-Containing Protein-1 (CDCP1);
D comprises a drug moiety;
n is an integer from 1 to 20; and
L has the formula:

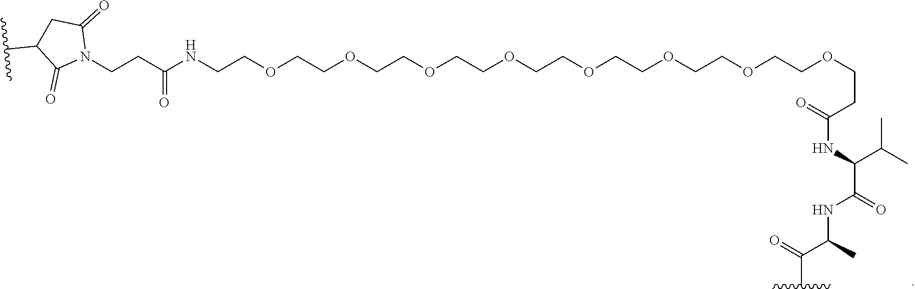 [SMILES: *C(=O)[C@H](C)NC(=O)[C@@H](NC(=O)CCOCCOCCOCCOCCOCCOCCOCCOCCNC(=O)CCN1C(=O)CC(*)C1=O)C(C)C]

In some embodiments, the antibody or binding fragment thereof comprises:
(i) a heavy chain variable region (VH) that comprises:
   (a) a VH complementarity determining region one (CDRH1) comprising and/or consisting of the amino acid sequence of SEQ ID NO: 2,
   (b) a VH complementarity determining region two (CDRH2) comprising and/or consisting of the amino acid sequence of SEQ ID NO: 3, and
   (c) a VH complementarity determining region three (CDRH3) comprising and/or consisting of the amino acid sequence of SEQ ID NO: 4,
and (ii) a light chain variable region (VL) that comprises:
   (a) a VL complementarity determining region one (CDRL1) comprising and/or consisting of the amino acid sequence of SEQ ID NO: 6,
   (b) a VL complementarity determining region two (CDRL2) comprising and/or consisting of the amino acid sequence of SEQ ID NO: 7, and
   (c) a VL complementarity determining region three (CDRL3) comprising and/or consisting of the amino acid sequence of SEQ ID NO:8.
      In some embodiments, the antibody or binding fragment thereof comprises:
(i) a heavy chain variable region (VH) that comprises:
   (a) a VH complementarity determining region one (CDRH1) comprising and/or consisting of the amino acid sequence of SEQ ID NO: 12,
   (b) a VH complementarity determining region two (CDRH2) comprising and/or consisting of the amino acid sequence of SEQ ID NO: 13, and
   (c) a VH complementarity determining region three (CDRH3) comprising and/or consisting of the amino acid sequence of SEQ ID NO: 14,
and (ii) a light chain variable region (VL) that comprises:
   (a) a VL complementarity determining region one (CDRL1) comprising and/or consisting of the amino acid sequence of SEQ ID NO: 6,
   (b) a VL complementarity determining region two (CDRL2) comprising and/or consisting of the amino acid sequence of SEQ ID NO: 7, and
   (c) a VL complementarity determining region three (CDRL3) comprising and/or consisting of the amino acid sequence of SEQ ID NO:8.
      In some embodiments, the drug moiety is selected from

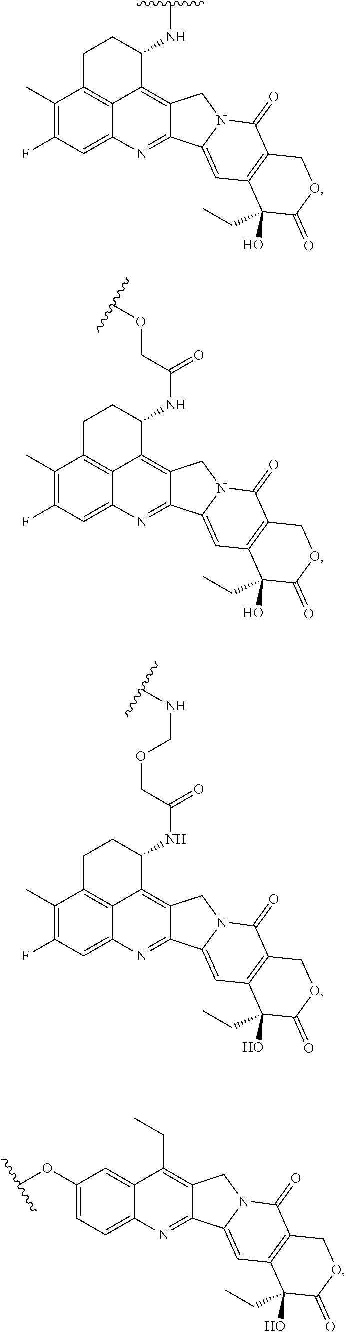 [SMILES: *N[C@H]1CCc2c(C)c(F)cc3nc4c(c1c23)Cn1c-4cc2c(c1=O)COC(=O)[C@]2(O)CC ; *OCC(=O)N[C@H]1CCc2c(C)c(F)cc3nc4c(c1c23)Cn1c-4cc2c(c1=O)COC(=O)[C@]2(O)CC ; *NCOCC(=O)N[C@H]1CCc2c(C)c(F)cc3nc4c(c1c23)Cn1c-4cc2c(c1=O)COC(=O)[C@]2(O)CC ; *Oc1ccc2nc3c(c(CC)c2c1)Cn1c-3cc2c(c1=O)COC(=O)[C@]2(O)CC]

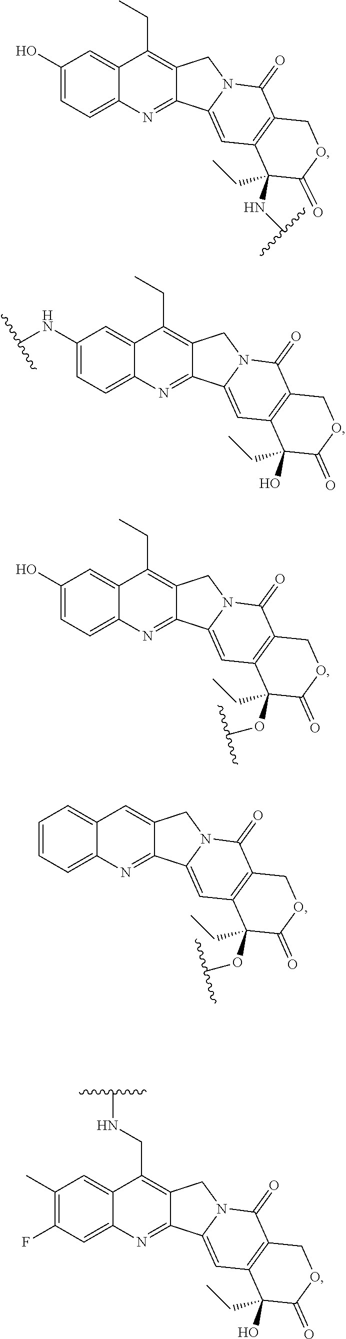 [SMILES: *N[C@]1(CC)C(=O)OCc2c1cc1n(c2=O)Cc2c-1nc1ccc(O)cc1c2CC ; *Nc1ccc2nc3c(c(CC)c2c1)Cn1c-3cc2c(c1=O)COC(=O)[C@]2(O)CC ; *O[C@]1(CC)C(=O)OCc2c1cc1n(c2=O)Cc2c-1nc1ccc(O)cc1c2CC ; *O[C@]1(CC)C(=O)OCc2c1cc1n(c2=O)Cc2cc3ccccc3nc2-1 ; *NCc1c2c(nc3cc(F)c(C)cc13)-c1cc3c(c(=O)n1C2)COC(=O)[C@]3(O)CC]

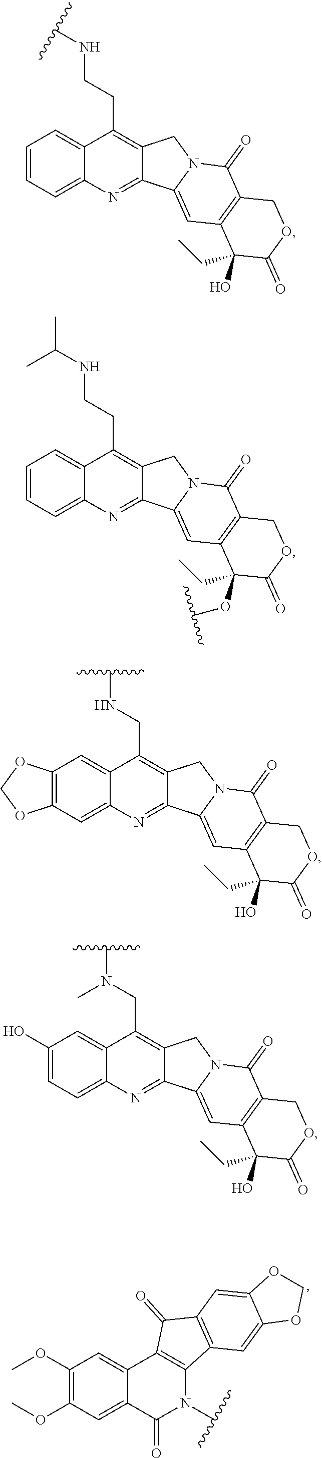 [SMILES: *NCCc1c2c(nc3ccccc13)-c1cc3c(c(=O)n1C2)COC(=O)[C@]3(O)CC ; *O[C@]1(CC)C(=O)OCc2c1cc1n(c2=O)Cc2c-1nc1ccccc1c2CCNC(C)C ; *NCc1c2c(nc3cc4c(cc13)OCO4)-c1cc3c(c(=O)n1C2)COC(=O)[C@]3(O)CC ; *N(C)Cc1c2c(nc3ccc(O)cc13)-c1cc3c(c(=O)n1C2)COC(=O)[C@]3(O)CC ; *n1c2c(c3cc(OC)c(OC)cc3c1=O)C(=O)c1cc3c(cc1-2)OCO3]

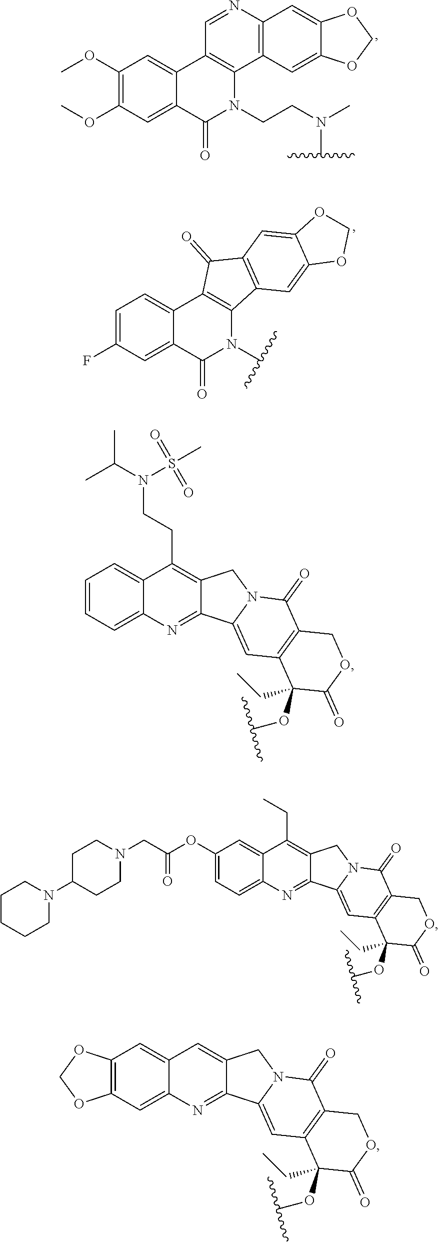 [SMILES: *N(C)CCn1c(=O)c2cc(OC)c(OC)cc2c2cnc3cc4c(cc3c21)OCO4 ; *n1c2c(c3ccc(F)cc3c1=O)C(=O)c1cc3c(cc1-2)OCO3 ; *O[C@]1(CC)C(=O)OCc2c1cc1n(c2=O)Cc2c-1nc1ccccc1c2CCN(C(C)C)S(C)(=O)=O ; *O[C@]1(CC)C(=O)OCc2c1cc1n(c2=O)Cc2c-1nc1ccc(OC(=O)CN3CCC(N4CCCCC4)CC3)cc1c2CC ; *O[C@]1(CC)C(=O)OCc2c1cc1n(c2=O)Cc2cc3cc4c(cc3nc2-1)OCO4]

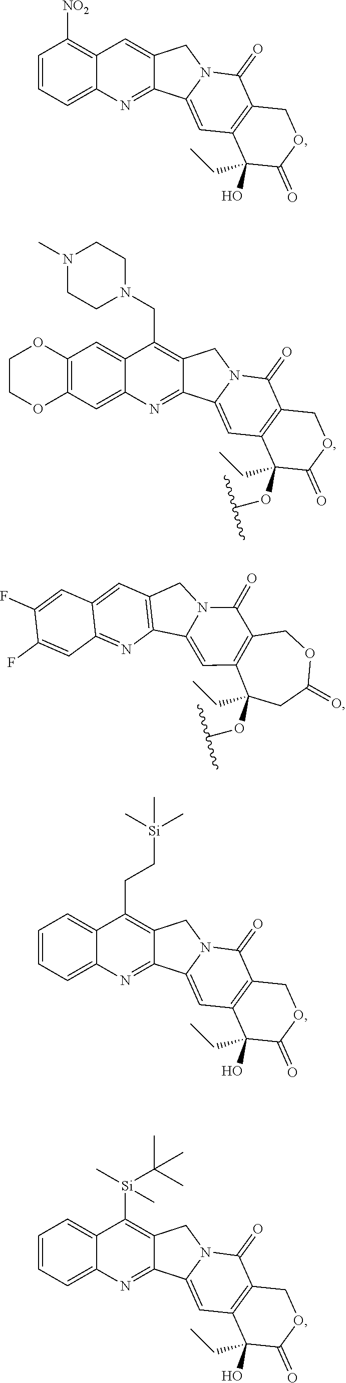 [SMILES: CC[C@@]1(O)C(=O)OCc2c1cc1n(c2=O)Cc2cc3c([N+](=O)[O-])cccc3nc2-1 ; *O[C@]1(CC)C(=O)OCc2c1cc1n(c2=O)Cc2c-1nc1cc3c(cc1c2CN1CCN(C)CC1)OCCO3 ; *O[C@]1(CC)CC(=O)OCc2c1cc1n(c2=O)Cc2cc3cc(F)c(F)cc3nc2-1 ; CC[C@@]1(O)C(=O)OCc2c1cc1n(c2=O)Cc2c-1nc1ccccc1c2CC[Si](C)(C)C ; CC[C@@]1(O)C(=O)OCc2c1cc1n(c2=O)Cc2c-1nc1ccccc1c2[Si](C)(C)C(C)(C)C]

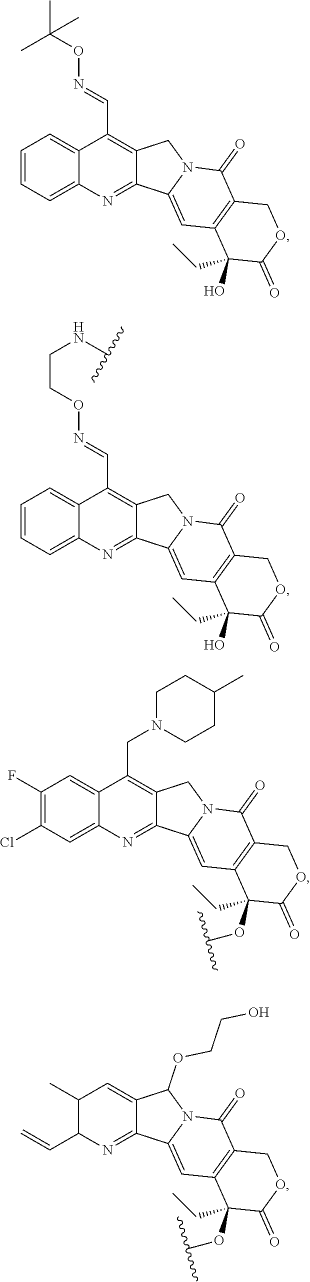

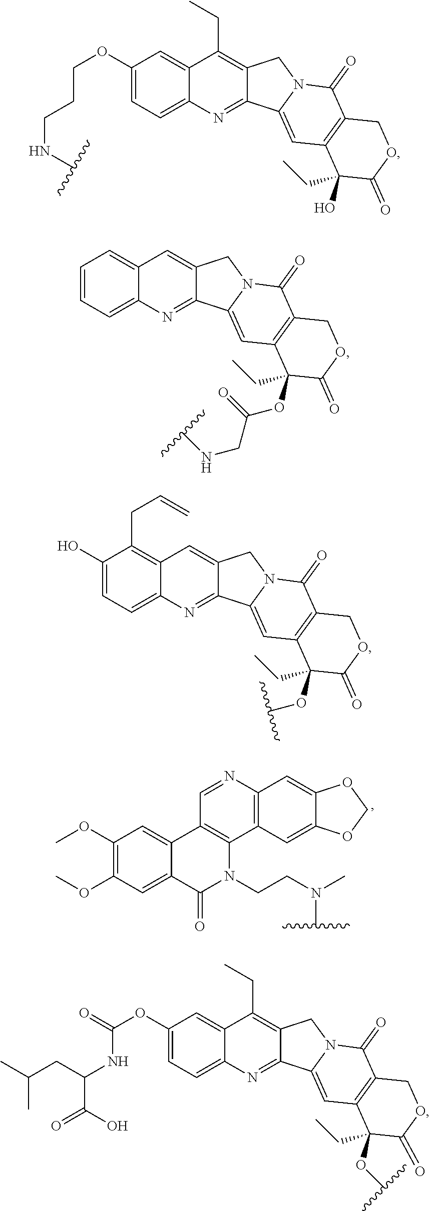

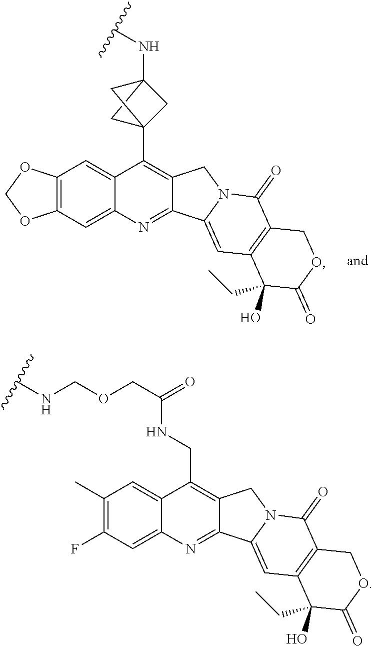 [SMILES: *NC12CC(c3c4c(nc5cc6c(cc35)OCO6)-c3cc5c(c(=O)n3C4)COC(=O)[C@]5(O)CC)(C1)C2 ; *NCOCC(=O)NCc1c2c(nc3cc(F)c(C)cc13)-c1cc3c(c(=O)n1C2)COC(=O)[C@]3(O)CC]

In some embodiments, the drug moiety is selected from

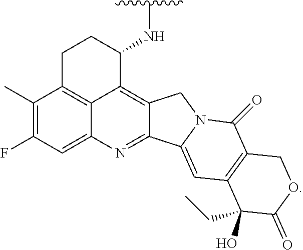 [SMILES: *N[C@H]1CCc2c(C)c(F)cc3nc4c(c1c23)Cn1c-4cc2c(c1=O)COC(=O)[C@]2(O)CC]

In some embodiments, the antibody-drug conjugate has a drug-to-antibody ratio (DAR) ranging from about 1 to about 10, optionally wherein the DAR is about 1, about 2, about 3, about 4, about 5, about 6, about 7, about 8, about 9, or about 10, optionally DAR is about 4, optionally DAR is about 8.

In aspects, the disclosure provides an antibody-drug conjugate having formula (I):

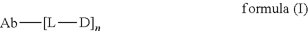

wherein in formula (I):
Ab comprises an antibody or antibody-binding fragment, wherein the antibody or binding fragment thereof specifically binds CUB Domain-Containing Protein-1 (CDCP1);
n is an integer from 1 to 20; and
L-D has the formula:

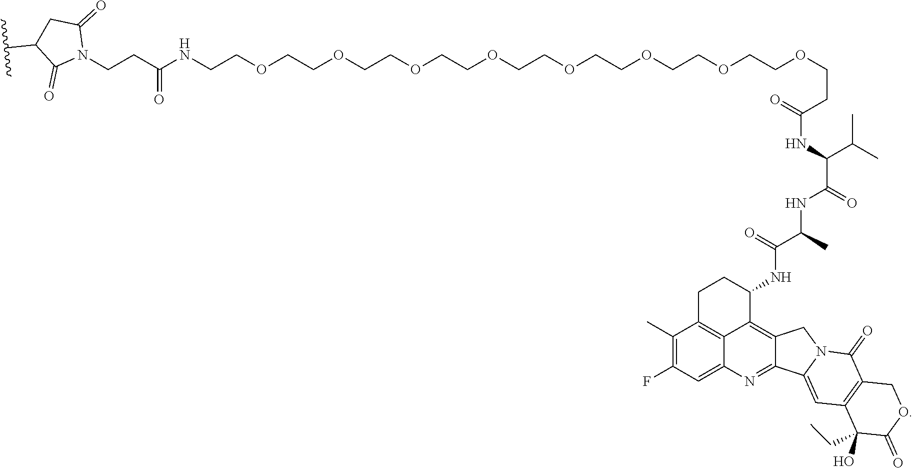 [SMILES: *C1CC(=O)N(CCC(=O)NCCOCCOCCOCCOCCOCCOCCOCCOCCC(=O)N[C@H](C(=O)N[C@@H](C)C(=O)N[C@H]2CCc3c(C)c(F)cc4nc5c(c2c34)Cn2c-5cc3c(c2=O)COC(=O)[C@]3(O)CC)C(C)C)C1=O]

In some embodiments, the antibody or binding fragment thereof comprises:
(i) a heavy chain variable region (VH) that comprises:
   (a) a VH complementarity determining region one (CDRH1) comprising and/or consisting of the amino acid sequence of SEQ ID NO: 2,
   (b) a VH complementarity determining region two (CDRH2) comprising and/or consisting of the amino acid sequence of SEQ ID NO: 3, and
   (c) a VH complementarity determining region three (CDRH3) comprising and/or consisting of the amino acid sequence of SEQ ID NO: 4,
and (ii) a light chain variable region (VL) that comprises:
   (a) a VL complementarity determining region one (CDRL1) comprising and/or consisting of the amino acid sequence of SEQ ID NO: 6,
   (b) a VL complementarity determining region two (CDRL2) comprising and/or consisting of the amino acid sequence of SEQ ID NO: 7, and
   (c) a VL complementarity determining region three (CDRL3) comprising and/or consisting of the amino acid sequence of SEQ ID NO:8.
      In some embodiments, the antibody or binding fragment thereof comprises:
(i) a heavy chain variable region (VH) that comprises:
   (a) a VH complementarity determining region one (CDRH1) comprising and/or consisting of the amino acid sequence of SEQ ID NO: 12,
   (b) a VH complementarity determining region two (CDRH2) comprising and/or consisting of the amino acid sequence of SEQ ID NO: 13, and
   (c) a VH complementarity determining region three (CDRH3) comprising and/or consisting of the amino acid sequence of SEQ ID NO: 14,
and (ii) a light chain variable region (VL) that comprises:
   (a) a VL complementarity determining region one (CDRL1) comprising and/or consisting of the amino acid sequence of SEQ ID NO: 6,
   (b) a VL complementarity determining region two (CDRL2) comprising and/or consisting of the amino acid sequence of SEQ ID NO: 7, and
   (c) a VL complementarity determining region three (CDRL3) comprising and/or consisting of the amino acid sequence of SEQ ID NO:8.
      In some embodiments, the antibody-drug conjugate has a drug-to-antibody ratio (DAR) ranging from about 1 to about 10, optionally wherein the DAR is about 1, about 2, about 3, about 4, about 5, about 6, about 7, about 8, about 9, or about 10, optionally DAR is about 4, optionally DAR is about 8. In some embodiments, n is an integer from 1 to 10, 2 to 8, or 4 to 8, optionally n is 1, 2, 3, 4, 5, 6, 7, 8, 9, or 10, optionally n is 4 or 8, optionally n is 4, optionally n is 8.

In aspects, the disclosure provides an antibody-drug conjugate having any one of formula 1030-1064 or 1100-1118, wherein Ab comprises an antibody or antibody-binding fragment, wherein the antibody or binding fragment thereof specifically binds CUB Domain-Containing Protein-1 (CDCP1).

In aspects, the disclosure provides an antibody-drug conjugate of any one of embodiments (I)-(XVII).

In aspects, the disclosure provides an antibody-drug conjugate having formula (I):

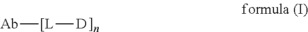

wherein in formula (I):
Ab comprises an antibody or antibody-binding fragment, wherein the antibody or binding fragment thereof specifically binds CUB Domain-Containing Protein-1 (CDCP1);
n is 1;
L-D has the formula:

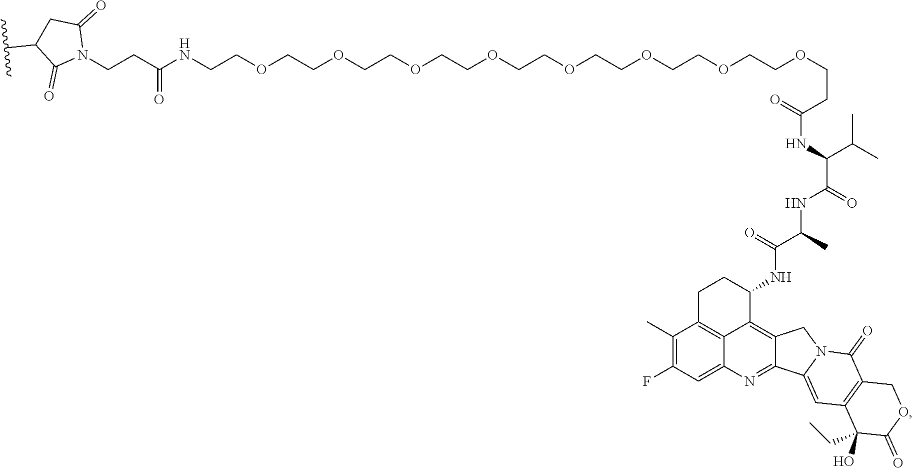 [SMILES: *C1CC(=O)N(CCC(=O)NCCOCCOCCOCCOCCOCCOCCOCCOCCC(=O)N[C@H](C(=O)N[C@@H](C)C(=O)N[C@H]2CCc3c(C)c(F)cc4nc5c(c2c34)Cn2c-5cc3c(c2=O)COC(=O)[C@]3(O)CC)C(C)C)C1=O]

wherein the antibody or binding fragment thereof comprises:
(i) a heavy chain variable region (VH) that comprises:
   (a) a VH complementarity determining region one (CDRH1) comprising and/or consisting of the amino acid sequence of SEQ ID NO: 2,
   (b) a VH complementarity determining region two (CDRH2) comprising and/or consisting of the amino acid sequence of SEQ ID NO: 3, and
   (c) a VH complementarity determining region three (CDRH3) comprising and/or consisting of the amino acid sequence of SEQ ID NO: 4,
and (ii) a light chain variable region (VL) that comprises:
   (a) a VL complementarity determining region one (CDRL1) comprising and/or consisting of the amino acid sequence of SEQ ID NO: 6,
   (b) a VL complementarity determining region two (CDRL2) comprising and/or consisting of the amino acid sequence of SEQ ID NO: 7, and
   (c) a VL complementarity determining region three (CDRL3) comprising and/or consisting of the amino acid sequence of SEQ ID NO:8.

In aspects, the disclosure provides an antibody-drug conjugate having formula (I):

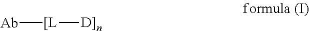

wherein in formula (I):
Ab comprises an antibody or antibody-binding fragment, wherein the antibody or binding fragment thereof specifically binds CUB Domain-Containing Protein-1 (CDCP1);
n is 4;
L-D has the formula:

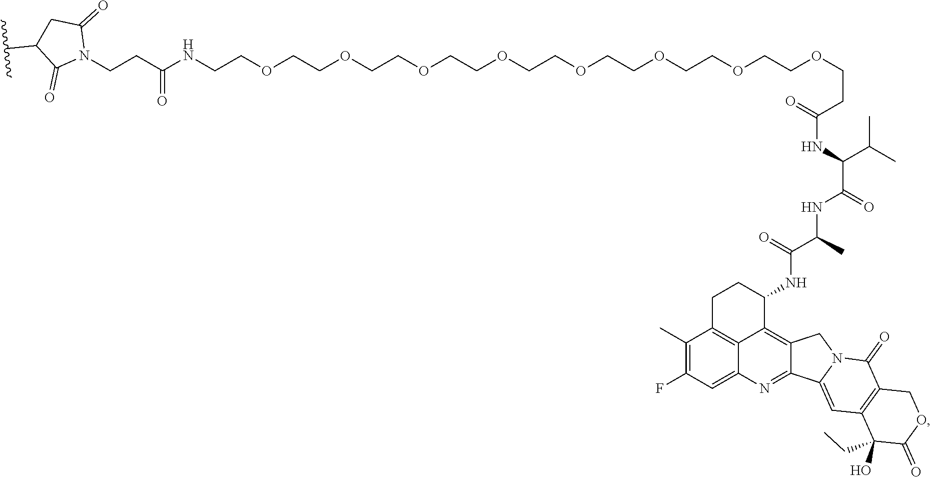 [SMILES: *C1CC(=O)N(CCC(=O)NCCOCCOCCOCCOCCOCCOCCOCCOCCC(=O)N[C@H](C(=O)N[C@@H](C)C(=O)N[C@H]2CCc3c(C)c(F)cc4nc5c(c2c34)Cn2c-5cc3c(c2=O)COC(=O)[C@]3(O)CC)C(C)C)C1=O]

wherein the antibody or binding fragment thereof comprises:
(i) a heavy chain variable region (VH) that comprises:
   (a) a VH complementarity determining region one (CDRH1) comprising and/or consisting of the amino acid sequence of SEQ ID NO: 2,
   (b) a VH complementarity determining region two (CDRH2) comprising and/or consisting of the amino acid sequence of SEQ ID NO: 3, and
   (c) a VH complementarity determining region three (CDRH3) comprising and/or consisting of the amino acid sequence of SEQ ID NO: 4,
and (ii) a light chain variable region (VL) that comprises:
   (a) a VL complementarity determining region one (CDRL1) comprising and/or consisting of the amino acid sequence of SEQ ID NO: 6,
   (b) a VL complementarity determining region two (CDRL2) comprising and/or consisting of the amino acid sequence of SEQ ID NO: 7, and
   (c) a VL complementarity determining region three (CDRL3) comprising and/or consisting of the amino acid sequence of SEQ ID NO:8.

In aspects, the disclosure provides an antibody-drug conjugate having formula (I):

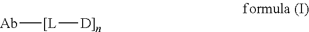

wherein in formula (I):
Ab comprises an antibody or antibody-binding fragment, wherein the antibody or binding fragment thereof specifically binds CUB Domain-Containing Protein-1 (CDCP1);
n is 8;
L-D has the formula:

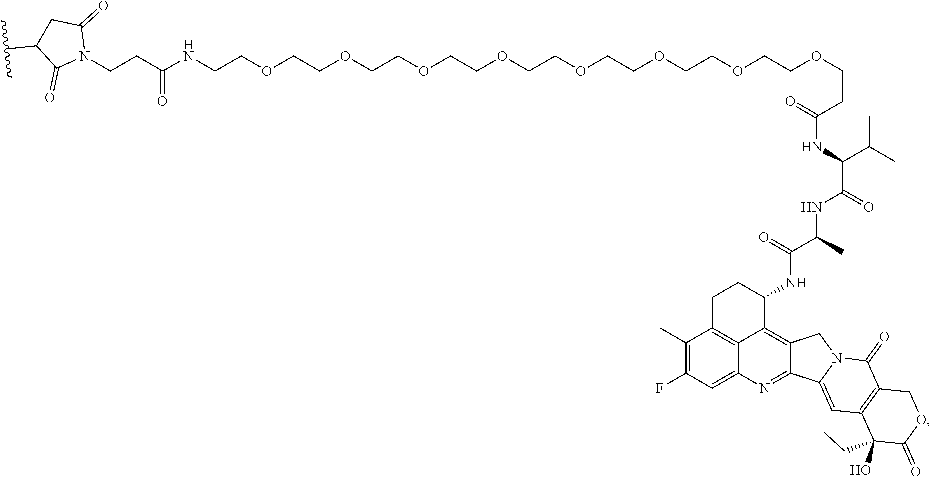 [SMILES: *C1CC(=O)N(CCC(=O)NCCOCCOCCOCCOCCOCCOCCOCCOCCC(=O)N[C@H](C(=O)N[C@@H](C)C(=O)N[C@H]2CCc3c(C)c(F)cc4nc5c(c2c34)Cn2c-5cc3c(c2=O)COC(=O)[C@]3(O)CC)C(C)C)C1=O]

wherein the antibody or binding fragment thereof comprises:
(i) a heavy chain variable region (VH) that comprises:
   (a) a VH complementarity determining region one (CDRH1) comprising and/or consisting of the amino acid sequence of SEQ ID NO: 2,
   (b) a VH complementarity determining region two (CDRH2) comprising and/or consisting of the amino acid sequence of SEQ ID NO: 3, and
   (c) a VH complementarity determining region three (CDRH3) comprising and/or consisting of the amino acid sequence of SEQ ID NO: 4,
and (ii) a light chain variable region (VL) that comprises:
   (a) a VL complementarity determining region one (CDRL1) comprising and/or consisting of the amino acid sequence of SEQ ID NO: 6,
   (b) a VL complementarity determining region two (CDRL2) comprising and/or consisting of the amino acid sequence of SEQ ID NO: 7, and
   (c) a VL complementarity determining region three (CDRL3) comprising and/or consisting of the amino acid sequence of SEQ ID NO:8.

In aspects, the disclosure provides an antibody-drug conjugate having formula (I):

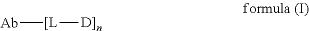

wherein in formula (I):
Ab comprises an antibody or antibody-binding fragment, wherein the antibody or binding fragment thereof specifically binds CUB Domain-Containing Protein-1 (CDCP1);
n is 1;
L-D has the formula:

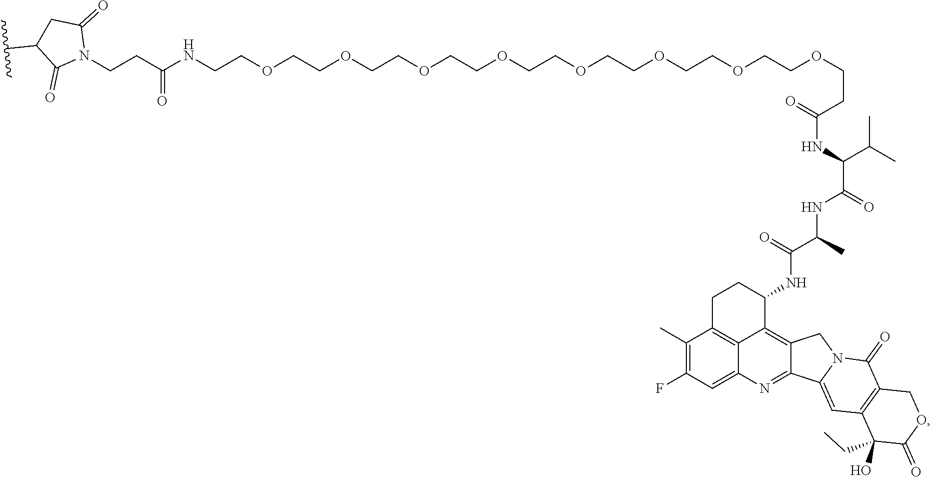 [SMILES: *C1CC(=O)N(CCC(=O)NCCOCCOCCOCCOCCOCCOCCOCCOCCC(=O)N[C@H](C(=O)N[C@@H](C)C(=O)N[C@H]2CCc3c(C)c(F)cc4nc5c(c2c34)Cn2c-5cc3c(c2=O)COC(=O)[C@]3(O)CC)C(C)C)C1=O]

wherein the antibody or binding fragment thereof comprises:
(i) a heavy chain variable region (VH) that comprises:
   (a) a VH complementarity determining region one (CDRH1) comprising and/or consisting of the amino acid sequence of SEQ ID NO: 12,
   (b) a VH complementarity determining region two (CDRH2) comprising and/or consisting of the amino acid sequence of SEQ ID NO: 13, and
   (c) a VH complementarity determining region three (CDRH3) comprising and/or consisting of the amino acid sequence of SEQ ID NO: 14,
and (ii) a light chain variable region (VL) that comprises:
   (a) a VL complementarity determining region one (CDRL1) comprising and/or consisting of the amino acid sequence of SEQ ID NO: 6,
   (b) a VL complementarity determining region two (CDRL2) comprising and/or consisting of the amino acid sequence of SEQ ID NO: 7, and
   (c) a VL complementarity determining region three (CDRL3) comprising and/or consisting of the amino acid sequence of SEQ ID NO:8.

In aspects, the disclosure provides an antibody-drug conjugate having formula (I):

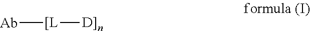

wherein in formula (I):
Ab comprises an antibody or antibody-binding fragment, wherein the antibody or binding fragment thereof specifically binds CUB Domain-Containing Protein-1 (CDCP1);
n is 4;
L-D has the formula:

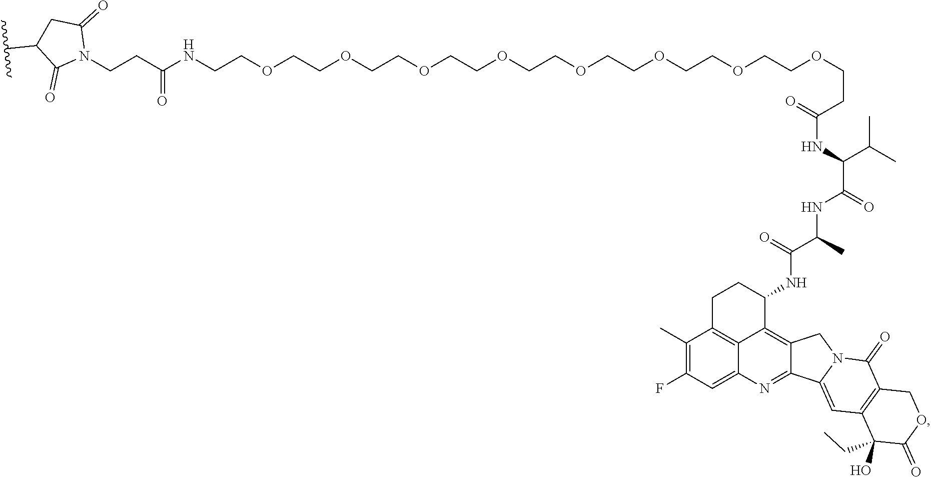 [SMILES: *C1CC(=O)N(CCC(=O)NCCOCCOCCOCCOCCOCCOCCOCCOCCC(=O)N[C@H](C(=O)N[C@@H](C)C(=O)N[C@H]2CCc3c(C)c(F)cc4nc5c(c2c34)Cn2c-5cc3c(c2=O)COC(=O)[C@]3(O)CC)C(C)C)C1=O]

wherein the antibody or binding fragment thereof comprises:
(i) a heavy chain variable region (VH) that comprises:
   (a) a VH complementarity determining region one (CDRH1) comprising and/or consisting of the amino acid sequence of SEQ ID NO: 12,
   (b) a VH complementarity determining region two (CDRH2) comprising and/or consisting of the amino acid sequence of SEQ ID NO: 13, and
   (c) a VH complementarity determining region three (CDRH3) comprising and/or consisting of the amino acid sequence of SEQ ID NO: 14,
and (ii) a light chain variable region (VL) that comprises:
   (a) a VL complementarity determining region one (CDRL1) comprising and/or consisting of the amino acid sequence of SEQ ID NO: 6,
   (b) a VL complementarity determining region two (CDRL2) comprising and/or consisting of the amino acid sequence of SEQ ID NO: 7, and
   (c) a VL complementarity determining region three (CDRL3) comprising and/or consisting of the amino acid sequence of SEQ ID NO:8.

In aspects, the disclosure provides an antibody-drug conjugate having formula (I):

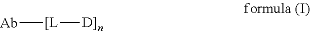

wherein in formula (I):
Ab comprises an antibody or antibody-binding fragment, wherein the antibody or binding fragment thereof specifically binds CUB Domain-Containing Protein-1 (CDCP1);
n is 8;
L-D has the formula:

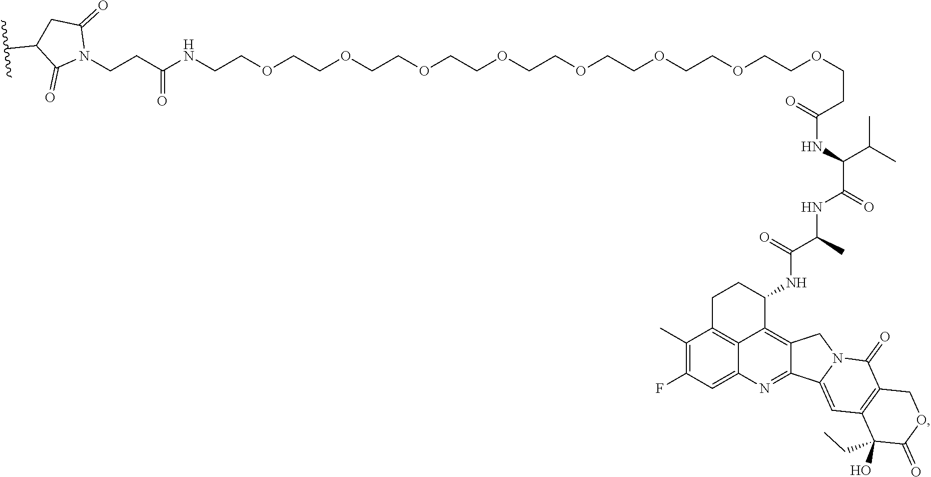 [SMILES: *C1CC(=O)N(CCC(=O)NCCOCCOCCOCCOCCOCCOCCOCCOCCC(=O)N[C@H](C(=O)N[C@@H](C)C(=O)N[C@H]2CCc3c(C)c(F)cc4nc5c(c2c34)Cn2c-5cc3c(c2=O)COC(=O)[C@]3(O)CC)C(C)C)C1=O]

wherein the antibody or binding fragment thereof comprises:
(i) a heavy chain variable region (VH) that comprises:
   (a) a VH complementarity determining region one (CDRH1) comprising and/or consisting of the amino acid sequence of SEQ ID NO: 12,
   (b) a VH complementarity determining region two (CDRH2) comprising and/or consisting of the amino acid sequence of SEQ ID NO: 13, and
   (c) a VH complementarity determining region three (CDRH3) comprising and/or consisting of the amino acid sequence of SEQ ID NO: 14,
and (ii) a light chain variable region (VL) that comprises:
   (a) a VL complementarity determining region one (CDRL1) comprising and/or consisting of the amino acid sequence of SEQ ID NO: 6,
   (b) a VL complementarity determining region two (CDRL2) comprising and/or consisting of the amino acid sequence of SEQ ID NO: 7, and
   (c) a VL complementarity determining region three (CDRL3) comprising and/or consisting of the amino acid sequence of SEQ ID NO:8.

In aspects, the disclosure provides a method of treating a cancer comprising administering to a subject in need thereof a therapeutically effective amount of the antibody-drug conjugate of formula (I).

### BRIEF DESCRIPTION OF THE DRAWINGS

The foregoing summary, as well as the following detailed description of embodiments of the disclosure, will be better understood when read in conjunction with the appended drawings and figures.

FIG. 1 illustrates a spectrum showing the SEC profile of CDCP1 mAb.

FIG. 2 illustrates a spectrum showing the HIC profile of CDCP1 mAb.

FIG. 3 illustrates a spectrum showing a PLRP trace of CDCP1 mAb. Heavy (H0) and light (L0) chain peaks are represented as peaks.

FIG. 4 illustrates a spectrum showing a spectrum of PLRP analysis used to assign DAR to the CDCP1-based ADC3. Light and heavy chains of the mAb are illustrated (L and H labels). Average DAR of 7.3 was calculated.

FIG. 5 illustrates a spectrum showing an SEC analysis used to purify the CDCP1-based ADC3. SEC analysis indicated monomeric purity of 97.5%.

FIG. 6 illustrates a spectrum showing a HIC profile of ADC4. Average DAR calculated as 4 with the DAR species assignments as indicated.

FIG. 7 illustrates a spectrum showing SEC analysis used to purify the CDCP1-based ADC4. SEC analysis indicated monomeric purity of 97.6%.

FIG. 8 illustrates a spectrum showing a PLRP analysis used to assign DAR to the CDCP1-based ADC5. Different colours represent different conjugation times (30, 60 and 90 min), with little difference in profile observed. Average DAR of 6.7 was calculated.

FIG. 9 illustrates a spectrum showing a SEC analysis used to purify the CDCP1-based ADC5. Different colours represent different conjugation times (30, 60 and 90 min), with little difference in profile observed and minimal unconjugated payload.

FIG. 10 is a graph illustrating experimental data demonstrating mean tumour volume versus time after one dose of ADC3 against MDA-MB-231 at 10 mg/kg and 6 mg/kg.

FIG. 11 is a graph illustrating experimental data demonstrating mean tumour volume versus time after three doses of ADC3 against MDA-MB-231 at 10 mg/kg, 6 mg/kg and 3 mg/kg.

FIG. 12A is a graph illustrating experimental data demonstrating PK Profile of mAb and ADC3 in male CD1 mouse plasma. FIG. 12B illustrates a comparison between multidose and single dose ADC3.

FIG. 13 is a graph illustrating experimental data demonstrating mean tumour volume versus time after one dose of ADC4 against MDA-MB-231 at 10 mg/kg.

FIG. 14 is a graph illustrating experimental data demonstrating mean tumour volume versus time after three doses of ADC4 against MDA-MB-231 at 10 mg/kg and 6 mg/kg.

FIG. 15A is a graph illustrating experimental data demonstrating PK profile of ADC4 at 10 mg/kg versus unconjugated mAb at the same dose in male CD-1 mouse plasma. PK profile of the ADC is favourable, with little difference in clearance between mAb and ADC observed. FIG. 15B illustrates experimental data demonstrating multidose administration of ADC4 in MDA-MB-231.

FIG. 16 illustrates experimental data demonstrating 10A-MMAE (DAR-4) produces complete regressions in MDA-MB-231 on a multi dose basis. The dose was increased to 5 mg/kg (multi-dose), and complete regressions have been observed. Antigen Copy Number of Target 10=~89,438.

FIG. 17 is an image showing a sequence of the labelled strand of the TyrT DNA fragment used in the cross-linking study.

FIG. 18 is an autoradiograph of a denaturing polyacrylamide gel investigating the mechanism of DNA interaction of 26 with linear 32P-end-labelled TyrT DNA following overnight incubation at 37° C. at various concentrations.

FIG. 19 is an autoradiograph of a denaturing polyacrylamide gel showing DNA interstrand cross-linking by the PBD dimer Talirine with linear 32P-end-labelled TyrT DNA following overnight incubation at 37° C. at various concentrations.

FIG. 20 is an image of a DNA footprint showing the interaction of multiple G-alkylators including 19 and 26 with the MS1 DNA fragment (left and centre left) and HexA (centre right), along with a DNA footprint illustrating the interaction of the PBD dimer Talirine with MS1 (right). Ligand concentrations are shown at the top of the gel. Tracks labelled “GA” are markers for specific purines.

FIG. 21 illustrates a sequence of the HexA DNA fragment showing the possible mono-alkylated adducts produced by the compounds analysed. Strong DNA footprints are represented by solid lines, and weaker footprints are represented by hatched lines

FIG. 22 illustrates a spectrum showing an HIC profile of ADC1. Average DAR calculated as 1.8 with the DAR (Drug Antibody Ratio) species assignments as indicated.

FIG. 23 illustrates a spectrum showing an SEC profile of ADC1; 94.3% monomer.

FIG. 24 illustrates a spectrum showing free toxin linker traces of the ADC1 sample. <2% free toxin linker could be detected in the ADC trace. Red: 100 μmol NAC-FGX11. Blue: CDCP1-27 after protein precipitation; the identified peaks show residual proteinaceous material.

FIG. 25 illustrates a spectrum showing the HIC profile of ADC2. Average DAR calculated as 4.2 with the DAR species assignments as indicated.

FIG. 26 illustrates a spectrum showing the SEC profile of ADC2; 94.4% monomer.

FIG. 27 illustrates spectrums showing free toxin linker traces of the ADC2 sample. 0.4% free toxin linker could be detected in the ADC trace. Red: 100 μmol NAC product Blue: ADC2 after protein precipitation; the identified peaks show residual proteinaceous material.

FIG. 28 illustrates a spectrum showing the HIC profile of ADC7. Average DAR calculated as 2.1 with the DAR species assignments as indicated.

FIG. 29 illustrates a spectrum showing an example of SEC analysis used to purify ADC7. The ADC contained 97.5% monomer.

FIG. 30 is a graph of experimental data illustrating binding of ADCs to antigen positive cell-line (A427). All ADCs has similar binding affinity compared to unconjugated CDCP1 mAb. Data also illustrate that unconjugated, non-targeted isotype control mAb did not bind to the antigen.

FIG. 31 is a graph of experimental data illustrating mean tumour volume versus time after one dose of ADC1 (Day 1) against K562. Dose dependent regression was observed.

FIG. 32 is a graph of experimental data illustrating PK Profile of mAb and ADC1 in male CD1 mouse plasma. PK profile of the ADC is favourable, with little difference in clearance between mAb and ADC observed.

FIG. 33 is a graph of experimental data illustrating mean tumour volume versus time after one dose of ADC2 (Day 1) against K562. Regression was observed with no weight loss.

FIG. 34 is a graph of experimental data illustrating mean tumour volume versus time after one dose of ADC2 (Day 1) against MDA-MB-231 at both 5 and 10 mg/kg. Complete regression was observed at the higher dose with no weight loss. Unconjugated mAb had negligible effect, indicating a targeted cell-killing ability of the ADC.

FIG. 35 is a graph of experimental data illustrating mean tumour volume versus time after multiple doses of ADC2 (either Days 1, 8 and 15 or Days 1, 22 and 43) against MDA-MB-231 at both 5 and 10 mg/kg. Complete regression was observed at the higher dose with no weight loss. Unconjugated mAb had negligible effect, indicating a targeted cell-killing ability of the ADC.

FIG. 36 is a graph of experimental data illustrating mean tumour volume versus time after a single dose of ADC2 (Day 1) against PC3 at doses from 1 mg/kg to 10 mg/kg. Concentration-dependent regressions were observed with no weight loss.

FIG. 37 is a graph of experimental data illustrating mean tumour volume versus time after three doses of ADC2 (days 1, 7 and 14) against A427 at 10 mg/kg.

FIG. 38 is a graph of experimental data illustrating PK Profile of mAb and ADC2 in male CD1 mouse plasma. PK profile of the ADC is favourable, with little difference in clearance between mAb and ADC observed.

FIG. 39A is a graph of experimental data illustrating mean tumour volume versus days post first dose (Q7dx3) of ADC3 (DAR of 8) at 3, 6, and 10 mg/kg and CDCP1-MMAE (DAR of 4) at 1, 3, and 6 mg/kg against NSCLC CALU-6. FIG. 39B is an immunohistochemistry (IHC) image showing expression of CDCP1 in the NSCLC cell-line.

FIG. 40A is a graph of experimental data illustrating mean tumour volume versus days post first dose (Q7dx3) of ADC3 (DAR of 8) at 3, 6, and 10 mg/kg and CDCP1-MMAE (DAR of 4) at 1, 3, and 6 mg/kg against TNBC MDA-MB-231. FIG. 40B is an IHC image showing expression of CDCP1 in the TNBC cell-line pre-treatment.

FIG. 41 is a summary of data from dose-range finding (DRF) and PK studies using CDCP1-vcMMAE (DAR of 4) at 4, 6, and 8 mg/kg and ADC3 (DAR of 8) at 15, 30, and 45 mg/kg in cynomolgus monkeys.

FIG. 42 is a framework for a non-limiting Good Laboratory Practice (GLP) toxicology study design framework for examining ADC3 (DAR of 8) in cynomolgus monkeys.

FIG. 43A is a graph of experimental data illustrating PK Profile of CDCP1-vcMMAE after 3 doses at 6 mg/kg compared to unconjugated mAb and unconjugated payload at the same doses in cynomolgus monkeys. FIG. 43B is a table summarizing additional PK data after 3 doses of CDCP1-vcMMAE at 8 mg/kg, 6 mg/kg, and 4 mg/kg in cynomolgus monkeys (in cyno plasma).

FIG. 44A is a graph of experimental data illustrating binding of unconjugated CDCP1-mAb to huCDCP1(F30-T667 Q525)-8×His_T3. FIG. 44B is a graph of experimental data illustrating binding of CDCP1-vcMMAE (DAR of 4) to huCDCP1 (F30-T667 Q525)-8×His_T3. FIG. 44C is a graph of experimental data illustrating binding of ADC2 (DAR of 4) to huCDCP1 (F30-T667 Q525)-8×His_T3. FIG. 44D is a graph of experimental data illustrating binding of ADC4 (DAR of 4) to huCDCP1 (F30-T667 Q525)-8×His_T3. FIG. 44E is a graph of experimental data illustrating binding of ADC3 (DAR 8) to huCDCP1 (F30-T667 Q525)-8×His_T3. FIG. 44F is a table summarizing additional binding data of unconjugated CDCP1-mAb and ADCs to recombinant CDCP1 ECD huCDCP1 (F30-T667 Q525)-8×His_T3. CDCP1-mAb=Sequence 1.

FIG. 45A is a graph of experimental data illustrating binding of unconjugated CDCP1-mAbs and CDCP1-vcMMAE to MDA-MB-468 cells after CDCP1+ cleavage. FIG. 45B is a graph of experimental data illustrating binding of unconjugated CDCP1-mAbs and CDCP1-vcMMAE to PC3 cells after CDCP1++ cleavage. FIG. 45C is a graph of experimental data illustrating binding of unconjugated CDCP1-mAbs and CDCP1-vcMMAE to DU145 cells after CDCP1+++ cleavage. FIG. 45D is a graph of experimental data illustrating binding of unconjugated CDCP1-mAbs and CDCP1-vcMMAE to OVMZ-6 cells after CDCP1 cleavage. FIG. 45E is an image of polyacrylamide gel binding assay.

FIG. 46A is a graph of experimental data illustrating binding affinity of unconjugated CDCP1-mAbs to CDCP1+ cells. FIG. 46B is a graph of experimental data illustrating binding affinity of ADC3 (DAR of 8) to CDCP1+ cells. FIG. 46C is a graph of experimental data illustrating binding affinity of CDCP1-vcMMAE (DAR of 4) to CDCP1+ cells.

FIG. 47A is a graph of experimental data illustrating relative cell survival (%) versus concentration of CDCP1-vcMMAE (DAR of 4). FIG. 47B is a graph of experimental data illustrating relative cell survival (%) versus concentration of ADC3 (DAR of 8).

FIG. 48A is a graph of experimental data illustrating relative cell survival (%) versus concentration of CDCP1-vcMMAE (DAR of 4). FIG. 48B is a graph of experimental data illustrating relative cell survival (%) versus concentration of ADC3 (DAR of 8).

FIG. 49A is an image of experimental data illustrating cytotoxicity of ADC3 (DAR of 8) in PC3 colony formation assay. FIG. 49B is an image of experimental data illustrating cytotoxicity of isotype-control exatecan in PC3 colony formation assay. FIG. 49C is a graph of experimental data illustrating colony formation (normalized to untreated colonies) versus concentration of unconjugated CDCP1-mAbs or ADC (μg/mL).

FIGS. 50A-50B show non-limiting examples of pharmacokinetic strategies for evaluating antibody drug conjugates of the disclosure. FIG. 50A shows a non-limiting example of a LC-MS based analysis for ADCs, total mAb and payload (e.g. CDCP1-vcMMAE). FIG. 50B shows a non-limiting example of a ELISA-based analysis for ADC, total mAb, with LC-MS for payload (e.g. ADC3).

FIGS. 51A-51B show experimental data illustrating efficacy and tolerability dat of ADC3. FIG. 51A shows a graph of experimental data demonstrating MDA-MB-231 (TNBC) treatment time compared to tumor volume (dosing day 0, 7, 14). Copy number=73,654 (FACS). FIG. 51B is a table of experimental data demonstrating a three dose (Q3W) non-human primate non-GLP toxicity study. Enhertu® Benchmarking Data: HNSTD=30 mg/kg, NOAEL=15 mg/kg (cyno); ILD observed in cyno at higher doses.

FIG. 52 illustrates experimental data demonstrating that ADC3 is highly stable in mouse/human/cyno plasma.

FIGS. 53A-53C illustrates experimental data demonstrating that in in vitro studies, trastuzumab-Compound 30 was found to be less potent in HER2+++ line than T-Dxd ADC (FIGS. 53A and 53C) despite free payloads being approximately equivalent in potency (FIG. 53B). FIG. 53A is a table showing EC50 values for compounds in a HER2+++ line. FIG. 53B illustrates the percent viable cells based on concentration of Dxd, Exatecan, or TOPO1 inhibitor control. FIG. 53C illustrates the percent viable cells based on concentration of ADC (trastuzumab-Compound 30, isotype-Compound 30, or trastuzumab-Dxd).

FIG. 54 illustrates experimental data demonstrating that in vivo efficacy shows more prolonged/sustained regressions with ADC3 compared to Enhertu®. JIMT-1 CDX in vivo efficacy (HER2+).

FIG. 55 illustrates a proposed mechanism of cleavage of the Val-Ala bond in compound 30.

FIG. 56 illustrates a table of experimental data demonstrating the rate of cleavage of various linker/payloads after reaction with papain at 24 hours and 96 hours.

FIG. 57 illustrates the structures of Compound 30, Compound 33, and AZ-0133.

FIG. 58 illustrates the structures of Deruxtecan and Compound 50.

FIG. 59 illustrates the structure of Trastuzumab-DM1 (Kadcyla®).

FIG. 60 illustrates structures of catabolites that were followed in liver and cyno microsome studies.

FIGS. 61A-61F are graphs of experimental data illustrating the formation of exatecan in incubations with trastuzumab-Compound 30 (FIG. 61A), trastuzumab-Compound 33 (FIG. 61B), trastuzumab-Compound 50 (FIG. 61C), trastuzumab-AZ-0133 (FIG. 61D), trastuzumab-deruxtecan (FIG. 61E), and trastuzumab-emtansine (FIG. 61F).

FIGS. 62A-62F are graphs of experimental data illustrating the formation of Cys-compound 20 (M1) in incubations with trastuzumab-Compound 30 (FIG. 62A); the formation of Cys-Mal-amido-PEG8-Val-Ala-PABC-exatecan in incubations with trastuzumab-Compound 33 (FIG. 62B); formation of Cys-Mal-amido-PEG8-exatecan in incubations with trastuzumab-Compound 50 (FIG. 62C); formation of Cys-Mal-amido-PEG8-Val-Ala-PABC-AZ-1033 in incubations with trastuzumab-AZ-0133 (FIG. 62D); formation of Cys-Mc-GGFG-DxD in incubations with trastuzumab-deruxtecan (FIG. 62E); and formation of Lys-MCC-DM1 in incubations with trastuzumab-emtansine (FIG. 62F).

### DETAILED DESCRIPTION

Unless defined otherwise, all technical and scientific terms used herein have the same meaning as is commonly understood by one of skill in the art to which this disclosure belongs. All patents and publications referred to herein are incorporated by reference in their entireties.

### Definitions

As used herein, the terms “administer,” “administration” or “administering” refer to (1) providing, giving, dosing, and/or prescribing by either a health practitioner or his authorized agent or under his or her direction according to the disclosure; and/or (2) putting into, taking or consuming by the mammal, according to the disclosure.

The terms “co-administration,” “co-administering,” “administered in combination with,” “administering in combination with,” “simultaneous,” and “concurrent,” as used herein, encompass administration of two or more active pharmaceutical ingredients to a subject so that both active pharmaceutical ingredients and/or their metabolites are present in the subject at the same time. Co-administration includes simultaneous administration in separate compositions, administration at different times in separate compositions, or administration in a composition in which two or more active pharmaceutical ingredients are present. Simultaneous administration in separate compositions and administration in a composition in which both agents are present are preferred.

The terms “active pharmaceutical ingredient” and “drug” antibodies, conjugates, and compounds described herein. The terms “active pharmaceutical ingredient” and “drug” may also include those compounds described herein that bind proteins, including but not limited to CDCP1, and thereby modulate protein activity.

The term “isostere” refers to a group or molecule whose chemical and/or physical properties are similar to those of another group or molecule. A “bioisostere” is a type of isostere and refers to a group or molecule whose biological properties are similar to those of another group or molecule. For example, a carboxylic acid may be replaced by one of the following bioisosteres for carboxylic acids, including, without limitation, alkyl esters (COOR), acylsulfonamides (CONR—SO₂R), hydroxamic acids (CONR—OH), hydroxamates (CONR—OR), tetrazoles, hydroxyisoxazoles, isoxazol-3-ones, and sulfonamides (SO₂NR), where each R may independently represent hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

The term “in vivo” refers to an event that takes place in a subject's body.

The term “in vitro” refers to an event that takes places outside of a subject's body. In vitro assays encompass cell-based assays in which cells alive or dead are employed and may also encompass a cell-free assay in which no intact cells are employed.

The term “effective amount” or “therapeutically effective amount” refers to that amount of a compound or combination of compounds as described herein that is sufficient to effect the intended application including, but not limited to, disease treatment. A therapeutically effective amount may vary depending upon the intended application (in vitro or in vivo), or the subject and disease condition being treated (e.g., the weight, age and gender of the subject), the severity of the disease condition, the manner of administration, etc., which can readily be determined by one of ordinary skill in the art. The term also applies to a dose that will induce a particular response in target cells (e.g., the reduction of platelet adhesion and/or cell migration). The specific dose will vary depending on the particular compounds chosen, the dosing regimen to be followed, whether the compound is administered in combination with other compounds, timing of administration, the tissue to which it is administered, and the physical delivery system in which the compound is carried.

A “therapeutic effect” as that term is used herein, encompasses a therapeutic benefit and/or a prophylactic benefit. A prophylactic effect includes delaying or eliminating the appearance of a disease or condition, delaying or eliminating the onset of symptoms of a disease or condition, slowing, halting, or reversing the progression of a disease or condition, or any combination thereof.

As used herein, the terms “treat,” “treatment,” and/or “treating” may refer to the management of a disease, disorder, or pathological condition, or symptom thereof with the intent to cure, ameliorate, stabilize, and/or control the disease, disorder, pathological condition or symptom thereof. Regarding control of the disease, disorder, or pathological condition more specifically, “control” may include the absence of condition progression, as assessed by the response to the methods recited herein, where such response may be complete (e.g., placing the disease in remission) or partial (e.g., lessening or ameliorating any symptoms associated with the condition). As used herein, the terms “prevent,” “preventing,” and/or “prevention” may refer to reducing the risk of developing a disease, disorder, or pathological condition.

As used herein, the terms “modulate” and “modulation” refer to a change in biological activity for a biological molecule (e.g., a protein, gene, peptide, antibody, and the like), where such change may relate to an increase in biological activity (e.g., increased activity, agonism, activation, expression, upregulation, and/or increased expression) or decrease in biological activity (e.g., decreased activity, antagonism, suppression, deactivation, downregulation, and/or decreased expression) for the biological molecule.

The terms “QD,” “qd,” or “q.d.” mean quaque die, once a day, or once daily. The terms “BID,” “bid,” or “b.i.d.” mean bis in die, twice a day, or twice daily. The terms “TID,” “tid,” or “t.i.d.” mean ter in die, three times a day, or three times daily. The terms “QID,” “qid,” or “q.i.d.” mean quater in die, four times a day, or four times daily.

The term “pharmaceutically acceptable salt” refers to salts derived from a variety of organic and inorganic counter ions known in the art. Pharmaceutically acceptable acid addition salts can be formed with inorganic acids and organic acids. Preferred inorganic acids from which salts can be derived include, for example, hydrochloric acid, hydrobromic acid, sulfuric acid, nitric acid and phosphoric acid. Preferred organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid and salicylic acid. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese and aluminum. Organic bases from which salts can be derived include, for example, primary, secondary, and tertiary amines, substituted amines including naturally occurring substituted amines, cyclic amines and basic ion exchange resins. Specific examples include isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts. The term “cocrystal” refers to a molecular complex derived from a number of cocrystal formers known in the art. Unlike a salt, a cocrystal typically does not involve hydrogen transfer between the cocrystal and the drug, and instead involves intermolecular interactions, such as hydrogen bonding, aromatic ring stacking, or dispersive forces, between the cocrystal former and the drug in the crystal structure.

“Pharmaceutically acceptable carrier” or “pharmaceutically acceptable excipient” or “physiologically compatible” carrier or carrier medium is intended to include any and all solvents, dispersion media, coatings, antibacterial and antifungal agents, isotonic and absorption delaying agents, and inert ingredients. The use of such pharmaceutically acceptable carriers or pharmaceutically acceptable excipients for active pharmaceutical ingredients is well known in the art. Except insofar as any conventional pharmaceutically acceptable carrier or pharmaceutically acceptable excipient is incompatible with the active pharmaceutical ingredient, its use in the therapeutic compositions of the disclosure is contemplated. Additional active pharmaceutical ingredients, such as other drugs, can also be incorporated into the described compositions and methods.

A “prodrug” refers to a derivative of a compound described herein, the pharmacologic action of which results from the conversion by chemical or metabolic processes in vivo to the active compound. Prodrugs include compounds wherein an amino acid residue, or a polypeptide chain of two or more (e.g., two, three or four) amino acid residues is covalently joined through an amide or ester bond to a free amino, hydroxyl or carboxylic acid group. The amino acid residues include but are not limited to the 20 naturally occurring amino acids commonly designated by one or three letter symbols but also include, for example, 4-hydroxyproline, hydroxylysine, desmosine, isodesmosine, 3-methylhistidine, beta-alanine, gamma-aminobutyric acid, citrulline, homocysteine, homoserine, ornithine and methionine sulfone. Additional types of prodrugs are also encompassed. For instance, free carboxyl groups can be derivatized as amides or alkyl esters (e.g., methyl esters and acetoxy methyl esters). Prodrug esters as employed herein includes esters and carbonates formed by reacting one or more hydroxyls of compounds of the method of the disclosure with alkyl, alkoxy, or aryl substituted acylating agents employing procedures known to those skilled in the art to generate acetates, pivalates, methylcarbonates, benzoates and the like. As further examples, free hydroxyl groups may be derivatized using groups including but not limited to hemisuccinates, phosphate esters, dimethylaminoacetates, and phosphoryloxymethyloxycarbonyls, as outlined in Advanced Drug Delivery Reviews, 1996, 19, 115. Carbamate prodrugs of hydroxyl and amino groups are also included, as are carbonate prodrugs, sulfonate prodrugs, sulfonate esters and sulfate esters of hydroxyl groups. Free amines can also be derivatized to amides, sulfonamides or phosphonamides. All of the stated prodrug moieties may incorporate groups including but not limited to ether, amine and carboxylic acid functionalities. Moreover, any compound that can be converted in vivo to provide the bioactive agent is a prodrug within the scope of the disclosure. Various forms of prodrugs are well known in the art. A comprehensive description of pro drugs and prodrug derivatives are described in: (a) The Practice of Medicinal Chemistry, Camille G. Wermuth et al., (Academic Press, 1996); (b) Design of Prodrugs, edited by H. Bundgaard, (Elsevier, 1985); (c) A Textbook of Drug Design and Development, P. Krogsgaard-Larson and H. Bundgaard, eds., (Harwood Academic Publishers, 1991). In general, prodrugs may be designed to improve the penetration of a drug across biological membranes in order to obtain improved drug absorption, to prolong duration of action of a drug (slow release of the parent drug from a prodrug, decreased first-pass metabolism of the drug), to target the drug action (e.g., organ or tumor-targeting, lymphocyte targeting), to modify or improve aqueous solubility of a drug (e.g., i.v. preparations and eyedrops), to improve topical drug delivery (e.g., dermal and ocular drug delivery), to improve the chemical/enzymatic stability of a drug, or to decrease off-target drug effects, and more generally in order to improve the therapeutic efficacy of the compounds utilized in the disclosure.

Unless otherwise stated, the chemical structures depicted herein are intended to include compounds which differ only in the presence of one or more isotopically enriched atoms. For example, compounds where one or more hydrogen atoms is replaced by deuterium or tritium, or wherein one or more carbon atoms is replaced by ¹³C- or ¹⁴C-enriched carbons, are within the scope of this disclosure.

“Alkyl” refers to a straight or branched hydrocarbon chain radical consisting solely of carbon and hydrogen atoms, containing no unsaturation, having from one to ten carbon atoms (e.g., (C₁₋₁₀)alkyl or C₁₋₁₀ alkyl). Whenever it appears herein, a numerical range such as “1 to 10” refers to each integer in the given range, e.g., “1 to 10 carbon atoms” means that the alkyl group may consist of 1 carbon atom, 2 carbon atoms, 3 carbon atoms, etc., up to and including 10 carbon atoms, although the definition is also intended to cover the occurrence of the term “alkyl” where no numerical range is specifically designated. Typical alkyl groups include, but are in no way limited to, methyl, ethyl, propyl, isopropyl, n-butyl, isobutyl, sec-butyl isobutyl, tertiary butyl, pentyl, isopentyl, neopentyl, hexyl, septyl, octyl, nonyl and decyl. The alkyl moiety may be attached to the rest of the molecule by a single bond, such as for example, methyl (Me), ethyl (Et), n-propyl (Pr), 1-methylethyl (isopropyl), n-butyl, n-pentyl, 1,1-dimethylethyl (t-butyl) and 3-methylhexyl. Unless stated otherwise specifically in the specification, an alkyl group is optionally substituted by one or more of substituents which are independently heteroalkyl, acylsulfonamido, alkenyl, alkynyl, cycloalkyl, heterocycloalkyl, hydroxamate, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —S(O)ₜR^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO(OR^{a})₂ where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Alkylaryl” refers to an -(alkyl) aryl radical where aryl and alkyl are as disclosed herein and which are optionally substituted by one or more of the substituents described as suitable substituents for aryl and alkyl respectively.

“Alkylhetaryl” refers to an -(alkyl) hetaryl radical where hetaryl and alkyl are as disclosed herein and which are optionally substituted by one or more of the substituents described as suitable substituents for aryl and alkyl respectively.

“Alkylheterocycloalkyl” refers to an -(alkyl) heterocyclic radical where alkyl and heterocycloalkyl are as disclosed herein and which are optionally substituted by one or more of the substituents described as suitable substituents for heterocycloalkyl and alkyl respectively.

An “alkene” moiety refers to a group consisting of at least two carbon atoms and at least one carbon-carbon double bond, and an “alkyne” moiety refers to a group consisting of at least two carbon atoms and at least one carbon-carbon triple bond. The alkyl moiety, whether saturated or unsaturated, may be branched, straight chain, or cyclic.

“Alkenyl” refers to a straight or branched hydrocarbon chain radical group consisting solely of carbon and hydrogen atoms, containing at least one double bond, and having from two to ten carbon atoms (i.e., (C₂₋₁₀)alkenyl or C₂₋₁₀ alkenyl). Whenever it appears herein, a numerical range such as “2 to 10” refers to each integer in the given range—e.g., “2 to 10 carbon atoms” means that the alkenyl group may consist of 2 carbon atoms, 3 carbon atoms, etc., up to and including 10 carbon atoms. The alkenyl moiety may be attached to the rest of the molecule by a single bond, such as for example, ethenyl (i.e., vinyl), prop-1-enyl (i.e., allyl), but-1-enyl, pent-1-enyl and penta-1,4-dienyl. Unless stated otherwise specifically in the specification, an alkenyl group is optionally substituted by one or more substituents which are independently alkyl, heteroalkyl, acylsulfonamido, alkenyl, alkynyl, cycloalkyl, heterocycloalkyl, hydroxamate, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —S(O)ₜR^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO(OR^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Alkenyl-cycloalkyl” refers to an -(alkenyl)cycloalkyl radical where alkenyl and cycloalkyl are as disclosed herein and which are optionally substituted by one or more of the substituents described as suitable substituents for alkenyl and cycloalkyl respectively.

“Alkynyl” refers to a straight or branched hydrocarbon chain radical group consisting solely of carbon and hydrogen atoms, containing at least one triple bond, having from two to ten carbon atoms (i.e., (C₂₋₁₀)alkynyl or C₂₋₁₀ alkynyl). Whenever it appears herein, a numerical range such as “2 to 10” refers to each integer in the given range—e.g., “2 to 10 carbon atoms” means that the alkynyl group may consist of 2 carbon atoms, 3 carbon atoms, etc., up to and including 10 carbon atoms. The alkynyl may be attached to the rest of the molecule by a single bond, for example, ethynyl, propynyl, butynyl, pentynyl and hexynyl. Unless stated otherwise specifically in the specification, an alkynyl group is optionally substituted by one or more substituents which independently are: alkyl, heteroalkyl, alkenyl, alkynyl, cycloalkyl, heterocycloalkyl, hydroxamate, acylsulfonamido, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —S(O)ₜR^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where tis 1 or 2), —S(O)ₜN(R^{a})₂ (where t is 1 or 2), or PO(OR^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Alkynyl-cycloalkyl” refers to an -(alkynyl)cycloalkyl radical where alkynyl and cycloalkyl are as disclosed herein and which are optionally substituted by one or more of the substituents described as suitable substituents for alkynyl and cycloalkyl respectively.

“Acylsulfonamide” refers to the group —C(═O)NR^{a}—S(═O)₂R^{a}, where each R^{a} is independently hydrogen, alkyl, fluoroalkyl, carbocyclyl, carbocyclylalkyl, aryl, aralkyl, heterocycloalkyl, heterocycloalkylalkyl, heteroaryl, or heteroarylalkyl.

“Carboxaldehyde” refers to a —(C═O)H radical.

“Carbonyl” refers to the group —C(═O)—. Carbonyl groups may be substituted with the following exemplary substituents: alkyl, heteroalkyl, alkenyl, alkynyl, cycloalkyl, heterocycloalkyl, hydroxamate, acylsulfonamido, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —S(O)ₜR^{a}— (where t is 1 or 2), —OC(O)—R^{a}, —N(R^{a})₂, —C(O)R^{a}, —NR^{a}—OR^{a}—, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO(OR^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Carboxyl” refers to a —(C═O) OH radical.

“Cyano” refers to a —CN radical.

“Cycloalkyl” refers to a monocyclic or polycyclic radical that contains only carbon and hydrogen, and may be saturated, or partially unsaturated. Cycloalkyl groups include groups having from 3 to 10 ring atoms (i.e., (C₃₋₁₀) cycloalkyl or C₃₋₁₀ cycloalkyl). Whenever it appears herein, a numerical range such as “3 to 10” refers to each integer in the given range—e.g., “3 to 10 carbon atoms” means that the cycloalkyl group may consist of 3 carbon atoms, etc., up to and including 10 carbon atoms. Illustrative examples of cycloalkyl groups include, but are not limited to the following moieties: cyclopropyl, cyclobutyl, cyclopentyl, cyclopentenyl, cyclohexyl, cyclohexenyl, cycloheptyl, cyclooctyl, cyclononyl, cyclodecyl, norbornyl, and the like. Unless stated otherwise specifically in the specification, a cycloalkyl group is optionally substituted by one or more substituents which independently are: alkyl, heteroalkyl, alkenyl, alkynyl, cycloalkyl, acylsulfonamido, heterocycloalkyl, hydroxamate, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —S(O)ₜR^{a}— (where t is 1 or 2), —S(O)ₜR^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO(OR^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Cycloalkyl-alkenyl” refers to a -(cycloalkyl)alkenyl radical where cycloalkyl and alkenyl are as disclosed herein and which are optionally substituted by one or more of the substituents described as suitable substituents for cycloalkyl and alkenyl, respectively.

“Cycloalkyl-heterocycloalkyl” refers to a -(cycloalkyl) heterocycloalkyl radical where cycloalkyl and heterocycloalkyl are as disclosed herein and which are optionally substituted by one or more of the substituents described as suitable substituents for cycloalkyl and heterocycloalkyl, respectively.

“Cycloalkyl-heteroaryl” refers to a -(cycloalkyl) heteroaryl radical where cycloalkyl and heteroaryl are as disclosed herein and which are optionally substituted by one or more of the substituents described as suitable substituents for cycloalkyl and heteroaryl, respectively.

The term “alkoxy” refers to the group-O-alkyl, including from 1 to 8 carbon atoms of a straight, branched, cyclic configuration and combinations thereof attached to the parent structure through an oxygen. Examples include, but are not limited to, methoxy, ethoxy, propoxy, isopropoxy, cyclopropyloxy and cyclohexyloxy. “Lower alkoxy” refers to alkoxy groups containing one to six carbons.

The term “substituted alkoxy” refers to alkoxy wherein the alkyl constituent is substituted (i.e., —O-(substituted alkyl)). Unless stated otherwise specifically in the specification, the alkyl moiety of an alkoxy group is optionally substituted by one or more substituents which independently are: alkyl, heteroalkyl, alkenyl, acylsulfonamido, alkynyl, cycloalkyl, heterocycloalkyl, hydroxamate, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —S(O)ₜR^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO(OR^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

The term “alkoxycarbonyl” refers to a group of the formula (alkoxy)(C═O)— attached through the carbonyl carbon wherein the alkoxy group has the indicated number of carbon atoms. Thus a (C₁₋₆)alkoxycarbonyl group is an alkoxy group having from 1 to 6 carbon atoms attached through its oxygen to a carbonyl linker. “Lower alkoxycarbonyl” refers to an alkoxycarbonyl group wherein the alkoxy group is a lower alkoxy group.

The term “substituted alkoxycarbonyl” refers to the group (substituted alkyl)-O—C(O)— wherein the group is attached to the parent structure through the carbonyl functionality. Unless stated otherwise specifically in the specification, the alkyl moiety of an alkoxycarbonyl group is optionally substituted by one or more substituents which independently are: alkyl, heteroalkyl, acylsulfonamido, alkenyl, alkynyl, cycloalkyl, heterocycloalkyl, hydroxamate, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —S(O)ₜR^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO(OR^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Acyl” refers to the groups (alkyl)-C(O)—, (aryl)-C(O)—, (heteroaryl)-C(O)—, (heteroalkyl)-C(O)— and (heterocycloalkyl)-C(O)—, wherein the group is attached to the parent structure through the carbonyl functionality. If the R radical is heteroaryl or heterocycloalkyl, the hetero ring or chain atoms contribute to the total number of chain or ring atoms. Unless stated otherwise specifically in the specification, the alkyl, aryl or heteroaryl moiety of the acyl group is optionally substituted by one or more substituents which are independently alkyl, heteroalkyl, acylsulfonamido, alkenyl, alkynyl, cycloalkyl, heterocycloalkyl, hydroxamate, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —S(O)ₜR^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO(OR^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Acyloxy” refers to a R(C═O)O— radical wherein R is alkyl, aryl, heteroaryl, heteroalkyl or heterocycloalkyl, which are as described herein. If the R radical is heteroaryl or heterocycloalkyl, the hetero ring or chain atoms contribute to the total number of chain or ring atoms. Unless stated otherwise specifically in the specification, the R of an acyloxy group is optionally substituted by one or more substituents which independently are: alkyl, heteroalkyl, alkenyl, alkynyl, cycloalkyl, heterocycloalkyl, hydroxamate, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —S(O)ₜR^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where tis 1 or 2), —S(O)ₜN(R^{a})₂ (where t is 1 or 2), or PO(OR^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Amino” or “amine” refers to a —N(R^{a})₂ radical group, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl, unless stated otherwise specifically in the specification. When a —N(R^{a})₂ group has two R^{a} substituents other than hydrogen, they can be combined with the nitrogen atom to form a 4-, 5-, 6- or 7-membered ring. For example, —N(R^{a})₂ is intended to include, but is not limited to, 1-pyrrolidinyl and 4-morpholinyl. Unless stated otherwise specifically in the specification, an amino group is optionally substituted by one or more substituents which independently are: alkyl, acylsulfonamido, heteroalkyl, alkenyl, alkynyl, cycloalkyl, heterocycloalkyl, hydroxamate, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —S(O)ₜR^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO(OR^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

The term “substituted amino” also refers to N-oxides of the groups —NHR^{d}, and NR^{d}R^{d} each as described above. N-oxides can be prepared by treatment of the corresponding amino group with, for example, hydrogen peroxide or m-chloroperoxybenzoic acid.

“Amide” or “amido” refers to a chemical moiety with formula —C(O)NR^{a}R^{b} or —NR^{a}C(O)R^{b}, where R^{a} and R^{b} are selected from the group consisting of hydrogen, alkyl, cycloalkyl, aryl, heteroaryl (bonded through a ring carbon) and heteroalicyclic (bonded through a ring carbon), each of which moiety may itself be optionally substituted. The R^{a} and R^{b} of —C(O)N R^{a}R^{b} amide may optionally be taken together with the nitrogen to which they are attached to form a 4-, 5-, 6- or 7-membered ring. Unless stated otherwise specifically in the specification, an amido group is optionally substituted independently by one or more of the substituents as described herein for alkyl, amino, cycloalkyl, aryl, heteroaryl, or heterocycloalkyl. An amide may be an amino acid or a peptide molecule attached to a compound disclosed herein, thereby forming a prodrug. The procedures and specific groups to make such amides are known to those of skill in the art and can readily be found in seminal sources such as Greene and Wuts, Protective Groups in Organic Synthesis, 3^{rd} Ed., John Wiley & Sons, New York, N.Y., 1999, which is incorporated herein by reference in its entirety.

“Aromatic” or “aryl” or “Ar” refers to an aromatic radical with six to ten ring atoms (e.g., C₆-C₁₀ aromatic or C₆-C₁₀ aryl) which has at least one ring having a conjugated pi electron system which is carbocyclic (e.g., phenyl, fluorenyl, and naphthyl). Bivalent radicals formed from substituted benzene derivatives and having the free valences at ring atoms are named as substituted phenylene radicals. Bivalent radicals derived from univalent polycyclic hydrocarbon radicals whose names end in “-yl” by removal of one hydrogen atom from the carbon atom with the free valence are named by adding “-idene” to the name of the corresponding univalent radical, e.g., a naphthyl group with two points of attachment is termed naphthylidene. Whenever it appears herein, a numerical range such as “6 to 10” refers to each integer in the given range; e.g., “6 to 10 ring atoms” means that the aryl group may consist of 6 ring atoms, 7 ring atoms, etc., up to and including 10 ring atoms. The term includes monocyclic or fused-ring polycyclic (i.e., rings which share adjacent pairs of ring atoms) groups. Unless stated otherwise specifically in the specification, an aryl moiety is optionally substituted by one or more substituents which are independently alkyl, heteroalkyl, acylsulfonamido, alkenyl, alkynyl, cycloalkyl, heterocycloalkyl, hydroxamate, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —S(O)ₜR^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where tis 1 or 2), —S(O)ₜN(R^{a})₂ (where t is 1 or 2), or PO(OR^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Aralkyl” or “arylalkyl” refers to an (aryl)alkyl-radical where aryl and alkyl are as disclosed herein and which are optionally substituted by one or more of the substituents described as suitable substituents for aryl and alkyl respectively.

“Ester” refers to a chemical radical of formula —COOR, where R is selected from the group consisting of alkyl, cycloalkyl, aryl, heteroaryl (bonded through a ring carbon) and heteroalicyclic (bonded through a ring carbon). The procedures and specific groups to make esters are known to those of skill in the art and can readily be found in seminal sources such as Greene and Wuts, Protective Groups in Organic Synthesis, 3^{rd} Ed., John Wiley & Sons, New York, N.Y., 1999, which is incorporated herein by reference in its entirety. Unless stated otherwise specifically in the specification, an ester group is optionally substituted by one or more substituents which independently are: alkyl, acylsulfonamido, heteroalkyl, alkenyl, alkynyl, cycloalkyl, heterocycloalkyl, hydroxamate, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —S(O)ₜR^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO(OR^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Fluoroalkyl” refers to an alkyl radical, as defined above, that is substituted by one or more fluoro radicals, as defined above, for example, trifluoromethyl, difluoromethyl, 2,2,2-trifluoroethyl, 1-fluoromethyl-2-fluoroethyl, and the like. The alkyl part of the fluoroalkyl radical may be optionally substituted as defined above for an alkyl group.

“Halo,” “halide,” or, alternatively, “halogen” is intended to mean fluoro, chloro, bromo or iodo. The terms “haloalkyl,” “haloalkenyl,” “haloalkynyl,” and “haloalkoxy” include alkyl, alkenyl, alkynyl and alkoxy structures that are substituted with one or more halo groups or with combinations thereof. For example, the terms “fluoroalkyl” and “fluoroalkoxy” include haloalkyl and haloalkoxy groups, respectively, in which the halo is fluorine.

“Heteroalkyl,” “heteroalkenyl,” and “heteroalkynyl” refer to optionally substituted alkyl, alkenyl and alkynyl radicals and which have one or more skeletal chain atoms selected from an atom other than carbon, e.g., oxygen, nitrogen, sulfur, phosphorus or combinations thereof. A numerical range may be given—e.g., C₁-C₄ heteroalkyl which refers to the chain length in total, which in this example is 4 atoms long. A heteroalkyl group may be substituted with one or more substituents which independently are: alkyl, heteroalkyl, alkenyl, alkynyl, acylsulfonamido, cycloalkyl, heterocycloalkyl, hydroxamate, aryl, arylalkyl, heteroaryl, heteroarylalkyl, hydroxy, halo, cyano, nitro, oxo, thioxo, trimethylsilanyl, —OR^{a}, —SR^{a}, —S(O)ₜR^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO(OR^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Heteroalkylaryl” refers to an -(heteroalkyl) aryl radical where heteroalkyl and aryl are as disclosed herein and which are optionally substituted by one or more of the substituents described as suitable substituents for heteroalkyl and aryl, respectively.

“Heteroalkylheteroaryl” refers to an -(heteroalkyl) heteroaryl radical where heteroalkyl and heteroaryl are as disclosed herein and which are optionally substituted by one or more of the substituents described as suitable substituents for heteroalkyl and heteroaryl, respectively.

“Heteroalkylheterocycloalkyl” refers to an -(heteroalkyl) heterocycloalkyl radical where heteroalkyl and heterocycloalkyl are as disclosed herein and which are optionally substituted by one or more of the substituents described as suitable substituents for heteroalkyl and heterocycloalkyl, respectively.

“Heteroalkylcycloalkyl” refers to an -(heteroalkyl)cycloalkyl radical where heteroalkyl and cycloalkyl are as disclosed herein and which are optionally substituted by one or more of the substituents described as suitable substituents for heteroalkyl and cycloalkyl, respectively.

“Heteroaryl” or “heteroaromatic” or “HetAr” refers to a 5- to 18-membered aromatic radical (e.g., C₅-C₁₃ heteroaryl) that includes one or more ring heteroatoms selected from nitrogen, oxygen and sulfur, and which may be a monocyclic, bicyclic, tricyclic or tetracyclic ring system. Whenever it appears herein, a numerical range such as “5 to 18” refers to each integer in the given range—e.g., “5 to 18 ring atoms” means that the heteroaryl group may consist of 5 ring atoms, 6 ring atoms, etc., up to and including 18 ring atoms. Bivalent radicals derived from univalent heteroaryl radicals whose names end in “-yl” by removal of one hydrogen atom from the atom with the free valence are named by adding “-idene” to the name of the corresponding univalent radical—e.g., a pyridyl group with two points of attachment is a pyridylidene. A N-containing “heteroaromatic” or “heteroaryl” moiety refers to an aromatic group in which at least one of the skeletal atoms of the ring is a nitrogen atom. The polycyclic heteroaryl group may be fused or non-fused. The heteroatom(s) in the heteroaryl radical are optionally oxidized. One or more nitrogen atoms, if present, are optionally quaternized. The heteroaryl may be attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzoxazolyl, benzopyranyl, benzopyranonyl, benzofuranyl, benzofuranonyl, benzofurazanyl, benzothiazolyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furazanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, isoxazol-3-one, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyranyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, thiapyranyl, triazolyl, tetrazolyl, triazinyl, thieno[2,3-d]pyrimidinyl, thieno[3,2-d]pyrimidinyl, thieno[2,3-c]pyridinyl, and thiophenyl (i.e. thienyl). Unless stated otherwise specifically in the specification, a heteroaryl moiety is optionally substituted by one or more substituents which are independently: alkyl, acylsulfonamido, heteroalkyl, alkenyl, alkynyl, cycloalkyl, heterocycloalkyl, hydroxamate, aryl, arylalkyl, heteroaryl, heteroarylalkyl, hydroxy, halo, cyano, nitro, oxo, thioxo, trimethylsilanyl, —OR^{a}, —SR^{a}, —S(O)ₜR^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where tis 1 or 2), —S(O)ₜN(R^{a})₂ (where t is 1 or 2), or PO(OR^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

Substituted heteroaryl also includes ring systems substituted with one or more oxide (—O—) substituents, such as, for example, pyridinyl N-oxides.

“Heteroarylalkyl” refers to a moiety having an aryl moiety, as described herein, connected to an alkylene moiety, as described herein, wherein the connection to the remainder of the molecule is through the alkylene group.

“Heterocycloalkyl” refers to a stable 3- to 18-membered non-aromatic ring radical that comprises two to twelve carbon atoms and from one to six heteroatoms selected from nitrogen, oxygen and sulfur. Whenever it appears herein, a numerical range such as “3 to 18” refers to each integer in the given range—e.g., “3 to 18 ring atoms” means that the heterocycloalkyl group may consist of 3 ring atoms, 4 ring atoms, etc., up to and including 18 ring atoms. Unless stated otherwise specifically in the specification, the heterocycloalkyl radical is a monocyclic, bicyclic, tricyclic or tetracyclic ring system, which may include fused or bridged ring systems. The heteroatoms in the heterocycloalkyl radical may be optionally oxidized. One or more nitrogen atoms, if present, are optionally quaternized. The heterocycloalkyl radical is partially or fully saturated. The heterocycloalkyl may be attached to the rest of the molecule through any atom of the ring(s). Examples of such heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocycloalkyl moiety is optionally substituted by one or more substituents which independently are: alkyl, acylsulfonamido, heteroalkyl, alkenyl, alkynyl, cycloalkyl, heterocycloalkyl, hydroxamate, aryl, arylalkyl, heteroaryl, heteroarylalkyl, hydroxy, halo, cyano, nitro, oxo, thioxo, trimethylsilanyl, —OR^{a}, —SR^{a}, —S(O)ₜR^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO(OR^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Heterocycloalkyl” also includes bicyclic ring systems wherein one non-aromatic ring, usually with 3 to 7 ring atoms, contains at least 2 carbon atoms in addition to 1-3 heteroatoms independently selected from oxygen, sulfur, and nitrogen, as well as combinations comprising at least one of the foregoing heteroatoms; and the other ring, usually with 3 to 7 ring atoms, optionally contains 1-3 heteroatoms independently selected from oxygen, sulfur, and nitrogen and is not aromatic.

“Hydroxamate” refers to the —C(O)NR^{a}OR^{a} moiety, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Nitro” refers to the —NO₂ radical.

“Oxa” refers to the —O-radical.

“Oxo” refers to the ═O radical.

“Isomers” are different compounds that have the same molecular formula. “Stereoisomers” are isomers that differ only in the way the atoms are arranged in space—i.e., having a different stereochemical configuration. “Enantiomers” are a pair of stereoisomers that are non-superimposable mirror images of each other. A 1:1 mixture of a pair of enantiomers is a “racemic” mixture. The term “(±)” is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer the stereochemistry at each chiral carbon can be specified by either (R) or (S). Resolved compounds whose absolute configuration is unknown can be designated (+) or (−) depending on the direction (dextro- or levorotatory) which they rotate plane polarized light at the wavelength of the sodium D line. Certain of the compounds described herein contain one or more asymmetric centers and can thus give rise to enantiomers, diastereomers, and other stereoisomeric forms that can be defined, in terms of absolute stereochemistry, as (R) or (S). The present chemical entities, pharmaceutical compositions and methods are meant to include all such possible isomers, including racemic mixtures, optically pure forms and intermediate mixtures. Optically active (R)- and (S)-isomers can be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers.

“Enantiomeric purity” as used herein refers to the relative amounts, expressed as a percentage, of the presence of a specific enantiomer relative to the other enantiomer. For example, if a compound, which may potentially have an (R)- or an (S)-isomeric configuration, is present as a racemic mixture, the enantiomeric purity is about 50% with respect to either the (R)- or (S)-isomer. If that compound has one isomeric form predominant over the other, for example, 80% (S)-isomer and 20% (R)-isomer, the enantiomeric purity of the compound with respect to the (S)-isomeric form is 80%. The enantiomeric purity of a compound can be determined in a number of ways known in the art, including but not limited to chromatography using a chiral support, polarimetric measurement of the rotation of polarized light, nuclear magnetic resonance spectroscopy using chiral shift reagents which include but are not limited to lanthanide containing chiral complexes or Pirkle's reagents, or derivatization of a compounds using a chiral compound such as Mosher's acid followed by chromatography or nuclear magnetic resonance spectroscopy.

In some embodiments, the enantiomerically enriched composition has a higher potency with respect to therapeutic utility per unit mass than does the racemic mixture of that composition. Enantiomers can be isolated from mixtures by methods known to those skilled in the art, including chiral high pressure liquid chromatography (HPLC) and the formation and crystallization of chiral salts; or preferred enantiomers can be prepared by asymmetric syntheses. See, for example, Jacques, et al., Enantiomers, Racemates and Resolutions, Wiley Interscience, New York (1981); E. L. Eliel, Stereochemistry of Carbon Compounds, McGraw-Hill, New York (1962); and E. L. Eliel and S. H. Wilen, Stereochemistry of Organic Compounds, Wiley-Interscience, New York (1994).

The terms “enantiomerically enriched” and “non-racemic,” as used herein, refer to compositions in which the percent by weight of one enantiomer is greater than the amount of that one enantiomer in a control mixture of the racemic composition (e.g., greater than 1:1 by weight). For example, an enantiomerically enriched preparation of the (S)-enantiomer, means a preparation of the compound having greater than 50% by weight of the (S)-enantiomer relative to the (R)-enantiomer, such as at least 75% by weight, or such as at least 80% by weight. In some embodiments, the enrichment can be significantly greater than 80% by weight, providing a “substantially enantiomerically enriched” or a “substantially non-racemic” preparation, which refers to preparations of compositions which have at least 85% by weight of one enantiomer relative to other enantiomer, such as at least 90% by weight, or such as at least 95% by weight. The terms “enantiomerically pure” or “substantially enantiomerically pure” refers to a composition that comprises at least 98% of a single enantiomer and less than 2% of the opposite enantiomer.

“Moiety” refers to a specific segment or functional group of a molecule. Chemical moieties are often recognized chemical entities embedded in or appended to a molecule.

“Tautomers” are structurally distinct isomers that interconvert by tautomerization. “Tautomerization” is a form of isomerization and includes prototropic or proton-shift tautomerization, which is considered a subset of acid-base chemistry. “Prototropic tautomerization” or “proton-shift tautomerization” involves the migration of a proton accompanied by changes in bond order, often the interchange of a single bond with an adjacent double bond. Where tautomerization is possible (e.g., in solution), a chemical equilibrium of tautomers can be reached. An example of tautomerization is keto-enol tautomerization. A specific example of keto-enol tautomerization is the interconversion of pentane-2,4-dione and 4-hydroxypent-3-en-2-one tautomers. Another example of tautomerization is phenol-keto tautomerization. A specific example of phenol-keto tautomerization is the interconversion of pyridin-4-ol and pyridin-4 (1H)-one tautomers.

A “leaving group or atom” is any group or atom that will, under selected reaction conditions, cleave from the starting material, thus promoting reaction at a specified site. Examples of such groups, unless otherwise specified, include halogen atoms and mesyloxy, p-nitrobenzensulphonyloxy and tosyloxy groups.

“Protecting group” is intended to mean a group that selectively blocks one or more reactive sites in a multifunctional compound such that a chemical reaction can be carried out selectively on another unprotected reactive site and the group can then be readily removed or deprotected after the selective reaction is complete. A variety of protecting groups are disclosed, for example, in T. H. Greene and P. G. M. Wuts, Protective Groups in Organic Synthesis, 3rd Edition, John Wiley & Sons, New York (1999).

“Solvate” refers to a compound in physical association with one or more molecules of a pharmaceutically acceptable solvent.

“Substituted” means that the referenced group may have attached one or more additional groups, radicals or moieties individually and independently selected from, for example, acyl, alkyl, alkylaryl, cycloalkyl, aralkyl, aryl, carbohydrate, carbonate, heteroaryl, heterocycloalkyl, hydroxamate, hydroxy, alkoxy, aryloxy, mercapto, alkylthio, arylthio, cyano, halo, carbonyl, ester, thiocarbonyl, isocyanato, thiocyanato, isothiocyanato, nitro, oxo, perhaloalkyl, perfluoroalkyl, phosphate, silyl, sulfinyl, sulfonyl, sulfonamidyl, sulfoxyl, sulfonate, urea, and amino, including mono- and di-substituted amino groups, and protected derivatives thereof. The substituents themselves may be substituted, for example, a cycloalkyl substituent may itself have a halide substituent at one or more of its ring carbons. The term “optionally substituted” means optional substitution with the specified groups, radicals or moieties.

“Sulfanyl” refers to groups that include —S-(optionally substituted alkyl), —S-(optionally substituted aryl), —S-(optionally substituted heteroaryl) and —S-(optionally substituted heterocycloalkyl).

“Sulfinyl” refers to groups that include —S(O)—H, —S(O)-(optionally substituted alkyl), —S(O)-(optionally substituted amino), —S(O)-(optionally substituted aryl), —S(O)-(optionally substituted heteroaryl) and —S(O)-(optionally substituted heterocycloalkyl).

“Sulfonyl” refers to groups that include —S(O₂)—H, —S(O₂)-(optionally substituted alkyl), —S(O₂)-(optionally substituted amino), —S(O₂)-(optionally substituted aryl), —S(O₂)-(optionally substituted heteroaryl), and —S(O₂)-(optionally substituted heterocycloalkyl).

“Sulfonamidyl” or “sulfonamido” refers to a —S(═O)₂—NRR radical, where each R is selected independently from the group consisting of hydrogen, alkyl, cycloalkyl, aryl, heteroaryl (bonded through a ring carbon) and heteroalicyclic (bonded through a ring carbon). The R groups in —NRR of the —S(═O)₂—NRR radical may be taken together with the nitrogen to which it is attached to form a 4-, 5-, 6- or 7-membered ring. A sulfonamido group is optionally substituted by one or more of the substituents described for alkyl, cycloalkyl, aryl, heteroaryl, respectively.

“Sulfoxyl” refers to a —S(═O)₂OH radical.

“Sulfonate” refers to a —S(═O)₂—OR radical, where R is selected from the group consisting of alkyl, cycloalkyl, aryl, heteroaryl (bonded through a ring carbon) and heteroalicyclic (bonded through a ring carbon). A sulfonate group is optionally substituted on R by one or more of the substituents described for alkyl, cycloalkyl, aryl, heteroaryl, respectively.

Compounds of the disclosure also include crystalline and amorphous forms of those compounds, including, for example, polymorphs, pseudopolymorphs, solvates, hydrates, unsolvated polymorphs (including anhydrates), conformational polymorphs, and amorphous forms of the compounds, as well as mixtures thereof. “Crystalline form” and “polymorph” are intended to include all crystalline and amorphous forms of the compound, including, for example, polymorphs, pseudopolymorphs, solvates, hydrates, unsolvated polymorphs (including anhydrates), conformational polymorphs, and amorphous forms, as well as mixtures thereof, unless a particular crystalline or amorphous form is referred to.

The term “antibody” herein is used in the broadest sense and encompasses various antibody structures, including but not limited to monoclonal antibodies, polyclonal antibodies, and multispecific antibodies (e.g., bispecific antibodies).

An exemplary antibody such as an IgG comprises two heavy chains and two light chains. Each heavy chain is comprised of a heavy chain variable region (abbreviated herein as VH) and a heavy chain constant region. Each light chain is comprised of a light chain variable region (abbreviated herein as VL) and a light chain constant region. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino terminus to carboxy-terminus in the following order: FR1, CDR1, FR2, CDR2, FR3, CDR3, FR4.

The hypervariable region generally encompasses amino acid residues from about amino acid residues 24-34 (LCDR1; “L” denotes light chain), 50-56 (LCDR2) and 89-97 (LCDR3) in the light chain variable region and around about 31-35B (HCDR1; “H” denotes heavy chain), 50-65 (HCDR2), and 95-102 (HCDR3) in the heavy chain variable region; Kabat et al., SEQUENCES OF PROTEINS OF IMMUNOLOGICAL INTEREST, 5th Ed. Public Health Service, National Institutes of Health, Bethesda, Md. (1991) and/or those residues forming a hypervariable loop (e.g. residues 26-32 (LCDR1), 50-52 (LCDR2) and 91-96 (LCDR3) in the light chain variable region and 26-32 (HCDR1), 53-55 (HCDR2) and 96-101 (HCDR3) in the heavy chain variable region; Chothia and Lesk (1987) J. Mol. Biol. 196:901-917.

The term “monoclonal antibody” as used herein refers to an antibody obtained from a population of substantially homogeneous antibodies, e.g., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies and is not to be construed as requiring production of the antibody by any method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “chimeric” antibody refers to a recombinant antibody in which a portion of the heavy and/or light chain is derived from a particular source or species, while the remainder of the heavy and/or light chain is derived from a different source or species.

A “human antibody” is an antibody that possesses an amino-acid sequence corresponding to that of an antibody produced by a human and/or has been made using any of the techniques for making human antibodies known to one of skill in the art. This definition of a human antibody specifically excludes a humanized antibody comprising non-human antigen-binding residues. Human antibodies can be produced using various techniques known in the art, including methods described in Cole et al, Monoclonal Antibodies and Cancer Therapy, Alan R. Liss, p. 77 (1985); Boerner et al, J. Immunol, 147 (I): 86-95 (1991). See also van Dijk and van de Winkel, Curr. Opin. Pharmacol, 5:368-74 (2001). Human antibodies can be prepared by administering the antigen to a transgenic animal that has been modified to produce such antibodies in response to antigenic challenge, but whose endogenous loci have been disabled, e.g., immunized HuMab mice (see, e.g., Nils Lonberg et al., 1994, Nature 368:856-859, WO 98/24884, WO 94/25585, WO 93/1227, WO 92/22645, WO 92/03918 and WO 01/09187 regarding HuMab mice), xenomice (see, e.g., U.S. Pat. Nos. 6,075,181 and 6,150,584 regarding XENOMOUSE™ technology) or Trianni mice (see, e.g., WO 2013/063391, WO 2017/035252 and WO 2017/136734).

The term “humanized antibody” refers to an antibody that has been engineered to comprise one or more human framework regions in the variable region together with non-human (e.g., mouse, rat, or hamster) complementarity-determining regions (CDRs) of the heavy and/or light chain. In certain embodiments, a humanized antibody comprises sequences that are entirely human except for the CDR regions. Humanized antibodies are typically less immunogenic to humans, relative to non-humanized antibodies, and thus offer therapeutic benefits in certain situations. Those skilled in the art will be aware of humanized antibodies and will also be aware of suitable techniques for their generation. See for example, Hwang, W. Y. K., et al., Methods 36:35, 2005; Queen et al., Proc. Natl. Acad. Sci. USA, 86:10029-10033, 1989; Jones et al., Nature, 321:522-25, 1986; Riechmann et al., Nature, 332:323-27, 1988; Verhoeyen et al., Science, 239:1534-36, 1988; Orlandi et al., Proc. Natl. Acad. Sci. USA, 86:3833-37, 1989; U.S. Pat. Nos. 5,225,539; 5,530,101; 5,585,089; 5,693,761; 5,693,762; 6,180,370; and Selick et al., WO 90/07861, each of which is incorporated herein by reference in its entirety.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, δ, ε, γ, and μ, respectively.

The terms “antigen-binding domain” of an antibody (or simply “binding domain”) of an antibody or similar terms refer to one or more fragments of an antibody that retain the ability to specifically bind to an antigen complex. Examples of binding fragments encompassed within the term “antigen-binding portion” of an antibody include (i) Fab fragments, monovalent fragments consisting of the VL, VH, CL and CH domains; (ii) F(ab′)2 fragments, bivalent fragments comprising two Fab fragments linked by a disulfide bridge at the hinge region; (iii) Fd fragments consisting of the VH and CH domains; (iv) Fv fragments consisting of the VL and VH domains of a single arm of an antibody, (v) dAb fragments (Ward et al., (1989) Nature 341:544-546), which consist of a VH domain; (vi) isolated complementarity determining regions (CDR), and (vii) combinations of two or more isolated CDRs which may optionally be joined by a synthetic linker.

“Complementarity determining region” or “CDR” as the terms are used herein refer to short polypeptide sequences within the variable region of both heavy and light chain polypeptides that are primarily responsible for mediating specific antigen recognition. There are three CDRs (termed CDR1, CDR2, and CDR3) within each VL and each VH.

As will be appreciated by those in the art, the exact numbering and placement of the CDRs can be different among different numbering systems. However, it should be understood that the disclosure of a variable heavy and/or variable light sequence includes the disclosure of the associated CDRs. Accordingly, the disclosure of each variable heavy region is a disclosure of the vhCDRs (e.g., vhCDR1, vhCDR2 and vhCDR3) and the disclosure of each variable light region is a disclosure of the vlCDRs (e.g., vlCDR1, vlCDR2 and vlCDR3).

In certain embodiments, the CDRs of an antibody can be determined according to the IMGT numbering system as described in Lefranc M-P, (1999) The Immunologist 7:132-136 and Lefranc M-P et al, (1999) Nucleic Acids Res 27:209-212, each of which is herein incorporated by reference in its entirety. Unless stated otherwise herein, references to residue numbers in the variable domain of antibodies means residue numbering by the IMGT numbering system.

In other embodiments, the CDRs of an antibody can be determined according to MacCallum R M et al, (1996) J Mol Biol 262:732-745, herein incorporated by reference in its entirety. See also, e.g., Martin A. “Protein Sequence and Structure Analysis of Antibody Variable Domains,” in Antibody Engineering, Kontermann and Diibel, eds., Chapter 31, pp. 422-439, Springer-Verlag, Berlin (2001), herein incorporated by reference in its entirety. In other embodiments, the CDRs of an antibody can be determined according to the AbM numbering scheme, which refers to AbM hypervariable regions, which represent a compromise between the Kabat CDRs and Chothia structural loops, and are used by Oxford Molecular's AbM antibody modeling software (Oxford Molecular Group, Inc.), herein incorporated by reference in its entirety.

“Framework” or “framework region” or “FR” refers to variable domain residues other than hypervariable region (HVR) residues. The FR of a variable domain generally consists of four FR domains: FR1, FR2, FR3, and FR4.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda Md. (1991), Vols. 1-3. In one embodiment, for the VL, the subgroup is subgroup kappa I as in Kabat et al., supra. In one embodiment, for the VH, the subgroup is subgroup Ill as in Kabat et al., supra.

The “hinge region” is generally defined as stretching from 216-238 (EU numbering) or 226-251 (Kabat numbering) of human IgG1. The hinge can be further divided into three distinct regions, the upper, middle (e.g., core), and lower hinge.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md. (1991).

A “blocking” antibody or an “antagonist” antibody is one which inhibits or reduces biological activity of the antigen it binds. Certain blocking antibodies or antagonist antibodies substantially or completely inhibit the biological activity of the antigen.

An “antibody that binds to the same epitope” as a reference antibody refers to an antibody that contacts an overlapping set of amino acid residues of the antigen as compared to the reference antibody or blocks binding of the reference antibody to its antigen in a competition assay by 50% or more. The amino acid residues of an antibody that contact an antigen can be determined, for example, by determining the crystal structure of the antibody in complex with the antigen or by performing hydrogen/deuterium exchange. In some embodiments, residues of an antibody that are within 5 Å the antigen are considered to contact the antigen. In some embodiments, an antibody that binds to the same epitope as a reference antibody blocks binding of the reference antibody to its antigen in a competition assay by 50% or more, and conversely, the reference antibody blocks binding of the antibody to its antigen in a competition assay by 50% or more.

The term “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab)2; diabodies; linear antibodies; single-chain antibody molecules (e.g., scFv). Papain digestion of antibodies produces two identical binding fragment thereofs, called “Fab” fragments, and a residual “Fc” fragment, a designation reflecting the ability to crystallize readily. The Fab fragment consists of an entire light (L) chain along with the variable region domain of the heavy (H) chain (VH), and the first constant domain of one heavy chain (CH1). Pepsin treatment of an antibody yields a single large F(ab)2 fragment which roughly corresponds to two disulfide linked Fab fragments having divalent antigen-binding activity and is still capable of cross-linking antigen. Fab fragments differ from Fab′ fragments by having additional few residues at the carboxy terminus of the CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

“Fv” consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody.

“Single-chain Fv” also abbreviated as “sFv” or “scFv” are antibody fragments that comprise the VH and VL antibody domains connected into a single polypeptide chain. Preferably, the sFv polypeptide further comprises a polypeptide linker between the VH and VL domains which enables the sFv to form the desired structure for antigen binding. For a review of sFv, see Plückthun in The Pharmacology of Monoclonal Antibodies, vol. 113, Rosenburg and Moore eds., Springer-Verlag, New York, pp. 269-315 (1994).

The term an “isolated antibody” when used to describe the various antibodies disclosed herein, means an antibody that has been identified and separated and/or recovered from a cell or cell culture from which it was expressed. Contaminant components of its natural environment are materials that would typically interfere with diagnostic or therapeutic uses for the polypeptide, and can include enzymes, hormones, and other proteinaceous or non-proteinaceous solutes. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC) approaches. For a review of methods for assessment of antibody purity, see, for example, Flatman et al., J. Chromatogr. B 848:79-87 (2007). In an embodiment, the antibody will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain.

With regard to the binding of an antibody to a target molecule, the term “specific binding” or “specifically binds to” or is “specific for” a particular polypeptide or an epitope on a particular polypeptide target means binding that is measurably different from a non-specific interaction. Specific binding can be measured, for example, by determining binding of a molecule compared to binding of a control molecule. For example, specific binding can be determined by competition with a control molecule that is similar to the target, for example, an excess of non-labeled target. In this case, specific binding is indicated if the binding of the labeled target to a probe is competitively inhibited by excess unlabeled target. The term “specific binding” or “specifically binds to” or is “specific for” a particular polypeptide or an epitope on a particular polypeptide target as used herein can be exhibited, for example, by a molecule having a Kd for the target of 10−4 M or lower, alternatively 10−5 M or lower, alternatively 10−6 M or lower, alternatively 10−7 M or lower, alternatively 10−8 M or lower, alternatively 10−9 M or lower, alternatively 10−10 M or lower, alternatively 10−11 M or lower, alternatively 10−12 M or lower or a Kd in the range of 10−4 M to 10−6 M or 10−6 M to 10−10 M or 10−7 M to 10−9 M. As will be appreciated by the skilled artisan, affinity and KD values are inversely related. A high affinity for an antigen is measured by a low KD value. In one embodiment, the term “specific binding” refers to binding where a molecule binds to a particular polypeptide or epitope on a particular polypeptide without substantially binding to any other polypeptide or polypeptide epitope. As used herein the terms “specific binding,” “specifically binds,” and “selectively binds,” refer to antibody binding to an epitope of CDCP1.

The term “affinity,” as used herein, means the strength of the binding of an antibody to an epitope. The affinity of an antibody is given by the dissociation constant Kd, defined as [Ab]×[Ag]/[Ab−Ag], where [Ab−Ag] is the molar concentration of the antibody-antigen complex, [Ab] is the molar concentration of the unbound antibody and [Ag] is the molar concentration of the unbound antigen. The affinity constant Ka is defined by 1/Kd. Methods for determining the affinity of mAbs can be found in Harlow, et al., Antibodies: A Laboratory Manual, Cold Spring Harbor Laboratory Press, Cold Spring Harbor, N.Y., 1988), Coligan et al., eds., Current Protocols in Immunology, Greene Publishing Assoc, and Wiley Interscience, N.Y., (1992, 1993), and Muller, Meth. Enzymol. 92:589-601 (1983), which references are entirely incorporated herein by reference. One standard method well known in the art for determining the affinity of mAbs is the use of surface plasmon resonance (SPR) screening (such as by analysis with a BIAcore™ SPR analytical device).

An “epitope” is a term of art that indicates the site or sites of interaction between an antibody and its antigen(s). As described by (Janeway, C, Jr., P. Travers, et al. (2001). Immunobiology: the immune system in health and disease. Part II, Section 3-8. New York, Garland Publishing, Inc.): “An antibody generally recognizes only a small region on the surface of a large molecule such as a protein . . . [Certain epitopes] are likely to be composed of amino acids from different parts of the [antigen] polypeptide chain that have been brought together by protein folding. Antigenic determinants of this kind are known as conformational or discontinuous epitopes because the structure recognized is composed of segments of the protein that are discontinuous in the amino acid sequence of the antigen but are brought together in the three-dimensional structure. In contrast, an epitope composed of a single segment of polypeptide chain is termed a continuous or linear epitope” (Janeway, C. Jr., P. Travers, et al. (2001). Immunobiology: the immune system in health and disease. Part II, Section 3-8. New York, Garland Publishing, Inc.).

The term “KD”, as used herein, is intended to refer to the dissociation constant of a particular antibody-antigen interaction. It is calculated by the formula: Koff/Kon=KD.

The term “IC50”, as used herein, is intended to refer to the effective concentration of antibody of the present invention needed to neutralize 50% of the bioactivity of IL-23 on human lymphoma DB cells in the bioassay described in Example 5: Inhibition of STAT3 activation in human DB cell Assay.

“EC50” with respect to an agent and a particular activity (e.g., binding to a cell, inhibition of enzymatic activity, activation or inhibition of an immune cell), refers to the efficient concentration of the agent which produces 50% of its maximum response or effect with respect to such activity. “EC100” with respect to an agent and a particular activity refers to the efficient concentration of the agent which produces its substantially maximum response with respect to such activity.

### Antibodies

In one aspect, the disclosure provides antibodies and antibody fragments useful within the antibody-drug conjugates (ADCs), linkers, and other compounds and/or conjugates described herein. In some embodiments, the antibody and/or antibody fragment binds to CDCP1. In some embodiments the antibody and/or antibody fragment is an antibody or antigen-binding portion thereof that is specific for CDCP1.

### CUB Domain-Containing Protein 1 (CDCP1)

CDCP1 (HGNC: 24357; NCBI Entrez Gene: 64866; Ensembl: ENSG00000163814; UniProtKB/Swiss-Prot: Q9H5V8) has a large extracellular domain (665 amino acids in size) containing three CUB domains that mediate protein-protein interactions and are likely involved in cell adhesion and interaction with the extracellular matrix. The CDCP1 gene has been found to be strongly expressed in cancer, and has been previously disclosed as a therapeutic target in at least WO 2020/097336 and WO 2018/112334, which are herein incorporated by reference in their entireties.

Transmembrane protein CDCP1 associates with Src and PKCδ and all three proteins display increases in tyrosine phosphorylation when CDCP1 is activated. Src phosphorylates and binds to CDCP1, followed by the binding of CDCP1 to the C2 domain which is part of the regulatory domain of PKCδ. Tyr-734 was identified as the site that is phosphorylated by Src and Src Family Kinases, and as such, P-Tyr-734 is a biomarker of CDCP1 activation. The full length CDCP1 protein is 135 kDa, but in some cells, the extracellular domain is proteolytically cleaved to a ~75 kDa transmembrane protein.

TABLE 1
Exemplary CDCP1 sequences

| SEQ | | |
|---|---|---|
| ID | | |
| NO: | Sequence name | Amino acid sequence |
| 9 | CUB domain-containing | MAGLNCGVSIALLGVLLLGAARLPRGAEAFEIALPRESNITVLIKLGTPT |
| | protein 1 isoform 1 | LLAKPCYIVISKRHITMLSIKSGERIVFTFSCQSPENHFVIEIQKNIDCM |
| | precursor | SGPCPFGEVQLQPSTSLLPTLNRTFIWDVKAHKSIGLELQFSIPRLRQIG |
| | [Homo sapiens] | PGESCPDGVTHSISGRIDATVVRIGTFCSNGTVSRIKMQEGVKMALHLPW |
| | | FHPRNVSGFSIANRSSIKRLCIIESVFEGEGSATLMSANYPEGFPEDELM |
| | | TWQFVVPAHLRASVSFLNFNLSNCERKEERVEYYIPGSTTNPEVFKLEDK |
| | | QPGNMAGNFNLSLQGCDQDAQSPGILRLQFQVLVQHPQNESNKIYVVDLS |
| | | NERAMSLTIEPRPVKQSRKFVPGCFVCLESRTCSSNLTLTSGSKHKISFL |
| | | CDDLTRLWMNVEKTISCTDHRYCQRKSYSLQVPSDILHLPVELHDFSWKL |
| | | LVPKDRLSLVLVPAQKLQQHTHEKPCNTSFSYLVASAIPSQDLYFGSFCP |
| | | GGSIKQIQVKQNISVTLRTFAPSFQQEASRQGLTVSFIPYFKEEGVFTVT |
| | | PDTKSKVYLRTPNWDRGLPSLTSVSWNISVPRDQVACLIFFKERSGVVCQ |
| | | TGRAFMIIQEQRTRAEEIFSLDEDVLPKPSFHHHSFWVNISNCSPTSGKQ |
| | | LDLLFSVTLTPRTVDLTVILIAAVGGGVLLLSALGLIICCVKKKKKKTNK |
| | | GPAVGIYNDNINTEMPRQPKKFQKGRKDNDSHVYAVIEDTMVYGHLLQDS |
| | | SGSFLQPEVDTYRPFQGTMGVCPPSPPTICSRAPTAKLATEEPPPRSPPE |
| | | SESEPYTFSHPNNGDVSSKDTDIPLLNTQEPMEPAE |

In some aspects, the CDCP1 is human CDCP1. In some aspects, the CDCP1 is cynomologus monkey (cyno) CDCP1. In some aspects, the CDCP1 is mouse CDCP1. In some aspects, the CDCP1 is primate CDCP1. An exemplary CDCP1 sequence is provided in Table 1.

### CDCP1 Antibodies

The term “antibody,” as used herein, encompasses the broadest sense and specifically covers monoclonal antibodies, polyclonal antibodies, dimers, multimers, multispecific antibodies (e.g., bispecific antibodies), and antibody fragments, so long as they exhibit the desired biological activity. Antibodies may be murine, human, humanized, chimeric, or derived from other species. An antibody is a protein generated by the immune system that is capable of recognizing and binding to a specific antigen. (Janeway, C., Travers, P., Walport, M., Shlomchik (2001) Immuno Biology, 5th Ed., Garland Publishing, New York). A target antigen generally has numerous binding sites, also called epitopes, recognized by CDRs on multiple antibodies. Each antibody that specifically binds to a different epitope has a different structure. Thus, one antigen may have more than one corresponding antibody. An antibody includes a full-length immunoglobulin molecule or an immunologically active portion of a full-length immunoglobulin molecule, i.e., a molecule that contains an antigen-binding site that immunospecifically binds an antigen of a target of interest or part thereof, such targets including but not limited to, cancer cell or cells that produce autoimmune antibodies associated with an autoimmune disease.

The immunoglobulin disclosed herein can be of any type (e.g., IgG, IgE, IgM, IgD, and IgA), class (e.g., IgG1, IgG2, IgG3, IgG4, IgA1 and IgA2) or subclass of immunoglobulin molecule. The immunoglobulins can be derived from any species. In one aspect, however, the immunoglobulin is of human, murine, or rabbit origin.

An “binding fragment thereof” of an antibody refers to a fragment of a full-length antibody that retains the ability to specifically bind to an antigen (preferably with substantially the same binding affinity). Examples of an binding fragment thereof includes (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., 1989 Nature 341:544-546), which consists of a VH domain; and (vi) an isolated complementarity determining region (CDR), disulfide-linked Fvs (dsFv), and anti-idiotypic (anti-Id) antibodies and intrabodies. Furthermore, although the two domains of the Fv fragment, VL and VH, are encoded by separate genes, they may be joined, using recombinant methods (e.g., by a synthetic linker) thus enabling them to be produced as a single protein chain in which the VL and VH regions pair to form monovalent molecules (known as single chain Fv (scFv)); see e.g., Bird et al., Science 242:423-426 (1988) and Huston et al., 1988, Proc. Natl. Acad. Sci. USA 85:5879-5883. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen-binding sites (see e.g., Holliger et al., 1993, Proc. Natl. Acad. Sci. USA 90:6444-6448; Poljak et al., 1994, Structure 2:1121-1123).

An antibody “variable domain” refers to the variable region of the antibody light chain (VL) or the variable region of the antibody heavy chain (VH), either alone or in combination. As known in the art, the variable regions of the heavy and light chains each consist of four framework regions (FR) connected by three complementarity determining regions (CDRs), and contribute to the formation of the antigen-binding site of antibodies.

“Complementarity Determining Regions” (CDRs) can be identified according to the definitions of the Kabat, Chothia, the accumulation of both Kabat and Chothia, AbM, contact, North, and/or conformational definitions or any method of CDR determination well known in the art. See, e.g., Kabat et al., 1991, Sequences of Proteins of Immunological Interest, 5th ed. (hypervariable regions); Chothia et al., 1989, Nature 342:877-883 (structural loop structures). The identity of the amino acid residues in a particular antibody that make up a CDR can be determined using methods well known in the art. AbM definition of CDRs is a compromise between Kabat and Chothia and uses Oxford Molecular's AbM antibody modeling software (Accelrys®).

The “contact” definition of CDRs is based on observed antigen contacts, set forth in MacCallum et al., 1996, J. Mol. Biol., 262:732-745. The “conformational” definition of CDRs is based on residues that make enthalpic contributions to antigen binding (see, e.g., Makabe et al., 2008, J. Biol. Chem., 283:1156-1166). North has identified canonical CDR conformations using a different preferred set of CDR definitions (North et al., 2011, J. Mol. Biol. 406:228-256). In another approach, referred to herein as the “conformational definition” of CDRs, the positions of the CDRs may be identified as the residues that make enthalpic contributions to antigen binding (Makabe et al., 2008, J Biol. Chem. 283:1156-1166).

Still other CDR boundary definitions may not strictly follow one of the above approaches, but will nonetheless overlap with at least a portion of the Kabat CDRs, although they may be shortened or lengthened in light of prediction or experimental findings that particular residues or groups of residues or even entire CDRs do not significantly impact antigen binding.

As used herein, a CDR may refer to CDRs defined by any approach known in the art, including combinations of approaches. The methods used herein may utilize CDRs defined according to any of these approaches. For any given embodiment containing more than one CDR, the CDRs (or other residue of the antibody) may be defined in accordance with any of Kabat, Chothia, North, extended, AbM, contact, and/or conformational definitions.

Residues in a variable domain are numbered according Kabat, which is a numbering system used for heavy chain variable domains or light chain variable domains of the compilation of antibodies. See, Kabat et al., 1991, Sequences of Proteins of Immunological Interest, 5th Ed. Public Health Service, National Institutes of Health, Bethesda, MD. Using this numbering system, the actual linear amino acid sequence may contain fewer or additional amino acids corresponding to a shortening of, or insertion into, a FR or CDR of the variable domain. For example, a heavy chain variable domain may include a single amino acid insert (residue 52a according to Kabat) after residue 52 of H2 and inserted residues (e.g., residues 82a, 82b, and 82c, according to Kabat) after heavy chain FR residue 82. The Kabat numbering of residues may be determined for a given antibody by alignment at regions of homology of the sequence of the antibody with a “standard” Kabat numbered sequence. Various algorithms for assigning Kabat numbering are available. The algorithm implemented in the version 2.3.3 release of Abysis (www.abysis.org) is used herein to assign Kabat numbering to variable regions CDRL1, CDRL2, CDRL3, CDRH1, CDRH2, and CDRH3.

In some embodiments, specific amino acid residue positions in an antibody may also be numbered according to Kabat. In some embodiments, an anti-CDCP1 targeting agent comprises one or more of the CDRs is listed in Table 2A and/or Table 2B.

“Framework” (FR) residues are antibody variable domain residues other than the CDR residues. A VH or VL domain framework comprises four framework sub-regions, FR1, FR2, FR3 and FR4, interspersed with CDRs in the following structure: FR1-CDR1-FR2-CDR2-FR3-CDR3-FR4.

An “epitope” refers to the area or region of an antigen to which an antibody specifically binds, e.g., an area or region comprising residues that interacts with the antibody. Epitopes can be linear or conformational.

The term “paratope” is derived from the above definition of “epitope” by reversing the perspective, and refers to the area or region of an antibody molecule which is involved in binding of an antigen, e.g., an area or region comprising residues that interacts with the antigen. A paratope may be linear or conformational (such as discontinuous residues in CDRs).

The epitope/paratope for a given antigen/antibody binding pair can be defined and characterized at different levels of detail using a variety of experimental and computational epitope mapping methods. The experimental methods include mutagenesis, X-ray crystallography, Nuclear Magnetic Resonance (NMR) spectroscopy, Hydrogen/deuterium exchange Mass Spectrometry (HX-MS) and various competition binding methods.

At its most detailed level, the epitope/paratope for the interaction between an antibody (Ab) and antigen (Ag) can be defined by the spatial coordinates defining the atomic contacts present in the Ag-Ab interaction, as well as information about their relative contributions to the binding thermodynamics. At one level, an epitope/paratope residue can be characterized by the spatial coordinates defining the atomic contacts between the Ag and Ab.

In one aspect, the epitope/paratope residue can be defined by a specific criterion, e.g., distance between atoms in the Ab and the Ag (e.g., a distance of equal to or less than about 4 Å from a heavy atom of the cognate antibody and a heavy atom of the antigen). In another aspect, an epitope/paratope residue can be characterized as participating in a hydrogen bond interaction with the cognate antibody/antigen, or with a water molecule that is also hydrogen bonded to the cognate antibody/antigen (water-mediated hydrogen bonding). In another aspect, an epitope/paratope residue can be characterized as forming a salt bridge with a residue of the cognate antibody/antigen. In yet another aspect, an epitope/paratope residue can be characterized as a residue having a non-zero change in buried surface area (BSA) due to interaction with the cognate antibody/antigen.

At a less detailed level, epitope/paratope can be characterized through function, e.g., by competition binding with other Abs. The epitope/paratope can also be defined more generically as comprising amino acid residues for which substitution by another amino acid will alter the characteristics of the interaction between the Ab and Ag (e.g., alanine scanning).

An antibody that “preferentially binds” or “specifically binds” (used interchangeably herein) to an epitope is a term well understood in the art, and methods to determine such specific or preferential binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. An antibody “specifically binds” or “preferentially binds” to a target if it binds with greater affinity, avidity, more readily, and/or with greater duration than it binds to other substances. In a non-limiting example, an antibody that specifically or preferentially binds to a CDCP1 epitope is an antibody that binds this epitope with greater affinity, avidity, more readily, and/or with greater duration than it binds to other CDCP1 epitopes or non-CDCP1 epitopes. It is also understood by reading this definition that, for example, an antibody (or moiety, targeting agent or epitope) which specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding.

“Specific binding” or “preferential binding” includes a compound, e.g., a protein, a nucleic acid, an antibody, and the like, which recognizes and binds to a specific molecule, but does not substantially recognize or bind other molecules in a sample. For instance, an antibody which recognizes and binds to its cognate antigen in a sample, but does not substantially recognize or bind other molecules in the sample, specifically binds to that cognate antigen. Thus, under designated assay conditions, the specified binding moiety (e.g., an antibody or an antigen-binding portion thereof) binds preferentially to a particular target molecule and does not bind in a significant amount to other components present in a test sample.

A variety of assays may be used to select an antibody or peptide that specifically binds a molecule of interest. For example, solid-phase ELISA immunoassay, immunoprecipitation, BIAcore™ (GE Healthcare, Piscataway, NJ), fluorescence-activated cell sorting (FACS), Octet™ (FortéBio, Inc., Menlo Park, CA) and Western blot analysis are among many assays that may be used to identify an antibody that specifically reacts with an antigen or a receptor, or ligand binding portion thereof, that specifically binds with a cognate ligand or binding partner. Typically, a specific or selective reaction will be at least twice background signal or noise and more typically more than 10 times background, even more specifically, an antibody is said to “specifically bind” an antigen when the equilibrium dissociation constant (K_{D}) value is ≤1 μM, such as ≤100 nM, ≤10 nM, ≤100 pM, ≤10 pM, or ≤1 pM.

The term “compete”, as used herein with regard to an antibody, means that binding of a first antibody, or an antigen-binding portion thereof, to an antigen reduces the subsequent binding of the same antigen by a second antibody or an antigen-binding portion thereof. In general, the binding a first antibody creates steric hindrance, conformational change, or binding to a common epitope (or portion thereof), such that the binding of the second antibody to the same antigen is reduced. Standard competition assays may be used to determine whether two antibodies compete with each other. One suitable assay for antibody competition involves the use of the Biacore technology, which can measure the extent of interactions using surface plasmon resonance (SPR) technology, typically using a biosensor system (such as a BIACORE® system). For example, SPR can be used in an in vitro competitive binding inhibition assay to determine the ability of one antibody to inhibit the binding of a second antibody. Another assay for measuring antibody competition uses an ELISA-based approach.

Furthermore, a high throughput process for “binding” antibodies based upon their competition is described in International Patent Application No. WO2003/48731, which is incorporated by reference herein in its entirety. Competition is present if one antibody (or fragment) reduces the binding of another antibody (or fragment) to a target (e.g. CDCP1). For example, a sequential binding competition assay may be used, with different antibodies being added sequentially. The first antibody may be added to reach binding that is close to saturation. Then, the second antibody is added. In a non-limiting example, if the binding of second antibody (e.g. the binding to CDCP1) is not detected, or is significantly reduced (e.g., at least about 10%, at least about 20%, at least about 30%, at least about 40%, at least about 50%, at least about 60%, at least about 70%, at least about 80%, or at least about 90% reduction) as compared to a parallel assay in the absence of the first antibody (which value can be set as 100%), the two antibodies are considered as competing with each other.

An “antigen-binding portion” (or interchangeably “binding fragment thereof”) comprises a portion of a full length antibody, generally the antigen-binding or variable region thereof. Examples of antigen-binding portions include Fab, Fab′, F(ab′)₂, and Fv fragments; diabodies; linear antibodies; fragments produced by a Fab expression library, anti-idiotypic (anti-Id) antibodies, CDR (complementary determining region), and epitope-binding fragments of any of the above which immunospecifically bind to cancer cell antigens, viral antigens or microbial antigens, single-chain antibody molecules; and multispecific antibodies formed from antibody fragments. In some embodiments, the antibody or antigen-binding portion thereof is selected from a monoclonal antibody, polyclonal antibody, antibody fragment, Fab, Fab′, Fab′-SH, F(ab′)2, Fv, single chain Fv, diabody, linear antibody, bispecific antibody, multispecific antibody, chimeric antibody, humanized antibody, human antibody, and fusion protein comprising the antigen-binding portion of an antibody.

The term “monoclonal antibody,” as used herein, refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., (1975) Nature 256:495, or may be made by recombinant DNA methods.

Fv is the minimum antibody fragment which contains a complete antigen-recognition and antigen-binding site. This region consists of a dimer of one heavy chain and one light chain variable domain in tight, non-covalent association. It is in this configuration that the three hypervariable regions of each variable domain interact to define an antigen-binding site on the surface of the VH-VL dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear at least one free thiol group. F(ab′)2 antibody fragments originally were produced as pairs of Fab′ fragments which have hinge cysteines between them. Other chemical couplings of antibody fragments are also known.

The light chains of antibodies from any vertebrate species can be assigned to one of two clearly distinct types, called kappa (κ) and lambda (λ), based on the amino acid sequences of their constant domains.

Single-chain Fv or scFv mean single chain variable region antibody fragments which comprise the VH and VL domains of antibody, wherein these domains are present in a single polypeptide chain. The Fv polypeptide may further comprise a polypeptide linker between the VH and VL domains which enables the scFv to form the desired structure for antigen-binding.

The term “diabodies” refers to small antibody fragments with two antigen-binding sites, which fragments comprise a variable heavy domain (VH) connected to a variable light domain (VL) in the same polypeptide chain (VH-VL). By using a linker that is too short to allow pairing between the two domains on the same chain, the domains are forced to pair with the complementary domains of another chain and create two antigen-binding sites.

Humanized forms of non-human (e.g., rodent) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues.

Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence.

An “isolated antibody” is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In some embodiments, the antibody may be purified (1) to greater than 95% by weight of antibody as determined by the Lowry method, or more than 99% by weight, (2) to a degree sufficient to obtain at least 15 residues of N-terminal or internal amino acid sequence by use of a spinning cup protein sequencer, or (3) to homogeneity by SDS-PAGE under reducing or non-reducing conditions using Coomassie blue or silver stain. Isolated antibody includes the antibody in situ within recombinant cells since at least one component of the antibody's natural environment will not be present. Ordinarily, however, isolated antibody will be prepared by at least one purification step.

In some embodiments, the targeting agent, antibody, or binding fragment thereof disclosed herein may comprise one or more conservative amino acid substitutions. A person of skill in the art will recognize that a conservative amino acid substitution is a substitution of one amino acid with another amino acid that has similar structural or chemical properties, such as, for example, a similar side chain. Exemplary conservative substitutions are described in the art, for example, in Watson et al., Molecular Biology of the Gene, The Benjamin/Cummings Publication Company, 4th Ed. (1987).

In some embodiments, the targeting agent, antibody, or binding fragment thereof comprises a variable heavy chain sequence that comprises an amino acid sequence with at least about 95%, about 96%, about 97%, about 98%, or about 99%, sequence identity to the amino acid sequence set forth in SEQ ID NO: 1. In other embodiments, the targeting agent, antibody, or binding fragment thereof retains the binding and/or functional activity of a targeting agent, antibody, or binding fragment thereof that comprises the variable heavy chain sequence of SEQ ID NOs: 2, 3, or 4. In still further embodiments, the targeting agent, antibody, or binding fragment thereof comprises the variable heavy chain sequence of SEQ ID NOs: 1, 2, 3, or 4 and has one or more conservative amino acid substitutions, e.g., 1, 2, 3, 4, 5, 1-2, 1-3, 1-4 or 1-5 conservative amino acid substitutions in the heavy chain variable sequence. In yet further embodiments, the one or more conservative amino acid substitutions fall within one or more framework regions in SEQ ID NOs: 1, 2, 3, or 4 (based on the numbering system of Kabat).

In particular embodiments, the targeting agent, antibody, or binding fragment thereof comprises a variable heavy chain sequence with at least about 95%, about 96%, about 97%, about 98%, or about 99% sequence identity to the binding protein heavy chain variable region sequence set forth in SEQ ID NOs: 1, 2, 3, or 4, comprises one or more conservative amino acid substitutions in a framework region (based on the numbering system of Kabat), and retains the binding and/or functional activity of a binding protein that comprises a variable heavy chain sequence as set forth in SEQ ID NOs: 1, 2, 3, or 4 and a variable light chain sequence as set forth in SEQ ID NOs: 5, 6, 7, or 8.

In some embodiments, the targeting agent, antibody, or binding fragment thereof a variable light chain sequence that comprises an amino acid sequence with at least about 95%, about 96%, about 97%, about 98%, or about 99%, sequence identity to the amino acid sequence set forth in SEQ ID NO: 5. In other embodiments, the targeting agent, antibody, or binding fragment thereof retains the binding and/or functional activity of a targeting agent, antibody, or binding fragment thereof that comprises the variable light chain sequence of SEQ ID Nos: 5, 6, 7, or 8. In still further embodiments, the targeting agent, antibody, or binding fragment thereof comprises the variable light chain sequence of SEQ ID NOs: 5, 6, 7, or 8 and has one or more conservative amino acid substitutions, e.g., 1, 2, 3, 4, 5, 1-2, 1-3, 1-4 or 1-5 conservative amino acid substitutions in the light chain variable sequence. In yet further embodiments, the one or more conservative amino acid substitutions fall within one or more framework regions in SEQ ID NOs: 5, 6, 7, or 8 (based on the numbering system of Kabat).

In particular embodiments, the targeting agent, antibody, or binding fragment thereof comprises a variable light chain sequence with at least about 95%, about 96%, about 97%, about 98%, or about 99% sequence identity to the binding protein light chain variable region sequence set forth in SEQ ID NOs: 5, 6, 7, or 8, comprises one or more conservative amino acid substitutions in a framework region (based on the numbering system of Kabat), and retains the binding and/or functional activity of a binding protein that comprises a variable heavy chain sequence as set forth in SEQ ID NOs: 1, 2, 3, or 4 and a variable light chain sequence as set forth in SEQ ID NOs: 5, 6, 7, or 8.

In one aspect, the disclosure provides targeting agents (e.g. antibodies and antibody fragments) that are useful within the conjugates (e.g. antibody-drug conjugates) and compounds of the disclosure. Any targeting agent (e.g. antibodies and antibody fragments) is contemplated by the present disclosure.

In some embodiments, the targeting agent (e.g. antibodies, and binding fragment thereofs thereof) specifically bind CDCP1. Sequences of exemplary antibodies are shown in WO 2018/112334, which is incorporated by reference herein in its entirety. In some embodiments, the ADC is used to treat cancer.

In some embodiments, the anti-CDCP1 targeting agent, antibody, or binding fragment thereof comprises a heavy chain variable region comprising a VH complementarity determining region three (CDRH3) having the amino acid sequence of SEQ ID NO: 4 and a light chain variable region comprising a VL complementarity determining region three (CDRL3) having the amino acid sequence of SEQ ID NO:8.

In some embodiments, the anti-CDCP1 targeting agent, antibody, or binding fragment thereof further comprises a heavy chain variable region comprising a VH complementarity determining region two (CDRH2) having the amino acid sequence of SEQ ID NO: 3 and a light chain variable region comprising a VL complementarity determining region two (CDRL2) having the amino acid sequence of SEQ ID NO:7.

In some embodiments, the anti-CDCP1 targeting agent, antibody, or binding fragment thereof comprises a heavy chain variable region comprising a VH complementarity determining region one (CDRH1) having the amino acid sequence of SEQ ID NO: 2 and a light chain variable region comprising a VL complementarity determining region one (CDRH1) having the amino acid sequence of either SEQ ID NO: 6.

TABLE 2A
Anti-CDCP1 sequences

| SEQ ID | Sequence | |
|---|---|---|
| NO: | identifier | Amino acid sequence |
| 1 | Heavy Chain | EVQLLESGGG LVQPGGSLRL SCAASGFTFS NYAMNWVRQA |
| | variable region | PGKGLEWVSA ISGGGGSTYY ADSVKGRFTI SRDNSKNTLY |
| | (VH) | LQMNSLRAED TAVYYCAKES ITMVRGVMDY YGMDVWGQGT |
| | | TVTVSS |
| 2 | CDRH1 | GFTFSNYAMN |
| 3 | CDRH2 | AISGGGGSTY YADSV |
| 4 | CDRH3 | AKESITMVRGVM DYYGMDV |
| 10 | Heavy Chain | EVQLLESGGG LVQPGGSLRL SCAASGFTFS NYAMNWVRQA |
| | | PGKGLEWVSA ISGGGGSTYY ADSVKGRFTI SRDNSKNTLY |
| | | LQMNSLRAED TAVYYCAKES ITMVRGVMDY YGMDVWGQGT |
| | | TVTVSSASTK GPSVFPLAPS SKSTSGGTAA LGCLVKDYFP |
| | | EPVTVSWNSG ALTSGVHTFP AVLQSSGLYS LSSVVTVPSS |
| | | SLGTQTYICN VNHKPSNTKV DKRVEPKSCD KTHTCPPCPA |
| | | PELLGGPSVF LFPPKPKDTL MISRTPEVTC VVVDVSHEDP |
| | | EVKFNWYVDG VEVHNAKTKP REEQYNSTYR VVSVLTVLHQ |
| | | DWLNGKEYKC KVSNKALPAP IEKTISKAKG QPREPQVYTL |
| | | PPSREEMTKN QVSLTCLVKG FYPSDIAVEW ESNGQPENNY |
| | | KTTPPVLDSD GSFFLYSKLT VDKSRWQQGN VFSCSVMHEA |
| | | LHNHYTQKSL SLSPG |
| 5 | Light Chain | DIQLTQSPSF LSASVGDRVT ITCRASQGIS SYLAWYQQKP |
| | variable region | GKAPKLLIYA ASTLQGGVPS RFSGSGSGTE FTLTISSLQP |
| | (VL) | EDFATYYCQH LNRFPRTFGQ GTKVEIK |
| 6 | CDRL1 | RASQGISSYL A |
| 7 | CDRL2 | AASTLQG |
| 8 | CDRL3 | QHLNRFPRT |
| 11 | Light Chain | DIQLTQSPSF LSASVGDRVT ITCRASQGIS SYLAWYQQKP |
| | | GKAPKLLIYA ASTLQGGVPS RFSGSGSGTE FTLTISSLQP |
| | | EDFATYYCQH LNRFPRTFGQ GTKVEIKRTV AAPSVFIFPP |
| | | SDEQLKSGTA SVVCLLNNFY PREAKVQWKV DNALQSGNSQ |
| | | ESVTEQDSKD STYSLSSTLT LSKADYEKHK VYACEVTHQG |
| | | LSSPVTKSFN RGEC |
| 12 | CDRH1 | NYAMN |
| 13 | CDRH2 | AISGGGGSTY YADSVKG |
| 14 | CDRH3 | ESITMVRGVM DYYGMDV |

TABLE 2B
Anti-CDCP1 sequences

| SEQ ID | Sequence | |
|---|---|---|
| NO: | identifier | Amino acid sequence |
| 15 | Heavy Chain | QVQLVQSGAE VKKPGSSVKV SCKASGGTFS SYAMSWVRQA PGQGLEWMGG |
| | variable region | IIPILGTTNY AQKFQGRVTI TADKSTSTAY MELSSLRSED TAVYYCAREG |
| | (VH) | LYAFDIWGQG TMVTVSS |
| 16 | CDRH1 | GGTFSSYAMS |
| 17 | CDRH2 | GIIPILGTTN YAQ |
| 18 | CDRH3 | AREGLYAFDI |
| 19 | Light Chain | DIQMTQSPSS LSASVGDRVT ITCRASQSIS SYLNWYQQKP GKAPKFLIYV |
| | variable region | ASSLQSGVPS RFSGRGSGTD FTLTISSLQP EDFATYYCQQ SYSTPWTFGQ |
| | (VL) | GTKVEIK |
| 20 | CDRL1 | RASQSISSYL N |
| 21 | CDRL2 | VASSLQS |
| 22 | CDRL3 | QQSYSTPWT |

In some embodiments, the targeting agent, antibody, or binding fragment thereof, comprises a heavy chain variable region (VH) that comprises an amino acid sequence at least about 90%, at least about 91%, at least about 92%, at least about 93%, at least about 94%, at least about 95%, at least about 96%, at least about 97%, at least about 98%, at least about 99%, or about 100% identical to the amino acid sequence of SEQ ID NO: 2.

In some embodiments, the targeting agent, antibody, or binding fragment thereof, comprises a VH that comprises an amino acid sequence at least about 90%, at least about 91%, at least about 92%, at least about 93%, at least about 94%, at least about 95%, at least about 96%, at least about 97%, at least about 98%, at least about 99%, or about 100% identical to the amino acid sequence of SEQ ID NO: 3.

In some embodiments, the targeting agent, antibody, or binding fragment thereof, comprises a VH that comprises an amino acid sequence at least about 90%, at least about 91%, at least about 92%, at least about 93%, at least about 94%, at least about 95%, at least about 96%, at least about 97%, at least about 98%, at least about 99%, or about 100% identical to the amino acid sequence of SEQ ID NO: 4.

In some embodiments, the targeting agent, antibody, or binding fragment thereof comprises a VL that comprises an amino acid sequence at least about 90%, at least about 91%, at least about 92%, at least about 93%, at least about 94%, at least about 95%, at least about 96%, at least about 97%, at least about 98%, at least about 99%, or about 100% identical to the amino acid sequence of SEQ ID NO: 6.

In some embodiments, the targeting agent, antibody, or binding fragment thereof comprises a light chain variable region (VL) that comprises an amino acid sequence at least about 90%, at least about 91%, at least about 92%, at least about 93%, at least about 94%, at least about 95%, at least about 96%, at least about 97%, at least about 98%, at least about 99%, or about 100% identical to the amino acid sequence of SEQ ID NO: 7.

In some embodiments, the targeting agent, antibody, or binding fragment thereof comprises a VL that comprises an amino acid sequence at least about 90%, at least about 91%, at least about 92%, at least about 93%, at least about 94%, at least about 95%, at least about 96%, at least about 97%, at least about 98%, at least about 99%, or about 100% identical to the amino acid sequence of SEQ ID NO: 8.

In some embodiments, the targeting agent, antibody, or binding fragment thereof comprises a VL that comprises an amino acid sequence at least about 90%, at least about 91%, at least about 92%, at least about 93%, at least about 94%, at least about 95%, at least about 96%, at least about 97%, at least about 98%, at least about 99%, or about 100% identical to the amino acid sequence of SEQ ID NO: 12.

In some embodiments, the targeting agent, antibody, or binding fragment thereof comprises a light chain variable region (VL) that comprises an amino acid sequence at least about 90%, at least about 91%, at least about 92%, at least about 93%, at least about 94%, at least about 95%, at least about 96%, at least about 97%, at least about 98%, at least about 99%, or about 100% identical to the amino acid sequence of SEQ ID NO: 13.

In some embodiments, the targeting agent, antibody, or binding fragment thereof comprises a VL that comprises an amino acid sequence at least about 90%, at least about 91%, at least about 92%, at least about 93%, at least about 94%, at least about 95%, at least about 96%, at least about 97%, at least about 98%, at least about 99%, or about 100% identical to the amino acid sequence of SEQ ID NO: 14.

In some embodiments, the targeting agent, antibody, or binding fragment thereof comprises a VH that comprises and/or consists of an amino acid sequence at least about 90%, at least about 91%, at least about 92%, at least about 93%, at least about 94%, at least about 95%, at least about 96%, at least about 97%, at least about 98%, at least about 99%, or about 100% identical to the amino acid sequence of SEQ ID NO: 1, and comprises a VL that comprises and/or consists of an amino acid sequence at least about 90%, at least about 91%, at least about 92%, at least about 93%, at least about 94%, at least about 95%, at least about 96%, at least about 97%, at least about 98%, at least about 99%, or about 100% identical to the amino acid sequence of SEQ ID NO: 5.

In some embodiments, the targeting agent, antibody, or binding fragment thereof comprises a VH that comprises and/or consists of an amino acid sequence that is 100% identical to the amino acid sequence of SEQ ID NO: 1.

In some embodiments, the targeting agent, antibody, or binding fragment thereof comprises a VL that comprises and/or consists of an amino acid sequence that is 100% identical to the amino acid sequence of SEQ ID NO: 5.

In some embodiments, the targeting agent, antibody, or binding fragment thereof comprises a VL that comprises an amino acid sequence at least about 90%, at least about 91%, at least about 92%, at least about 93%, at least about 94%, at least about 95%, at least about 96%, at least about 97%, at least about 98%, at least about 99%, or about 100% identical to the amino acid sequence of SEQ ID NO: 16.

In some embodiments, the targeting agent, antibody, or binding fragment thereof comprises a light chain variable region (VL) that comprises an amino acid sequence at least about 90%, at least about 91%, at least about 92%, at least about 93%, at least about 94%, at least about 95%, at least about 96%, at least about 97%, at least about 98%, at least about 99%, or about 100% identical to the amino acid sequence of SEQ ID NO: 17.

In some embodiments, the targeting agent, antibody, or binding fragment thereof comprises a VL that comprises an amino acid sequence at least about 90%, at least about 91%, at least about 92%, at least about 93%, at least about 94%, at least about 95%, at least about 96%, at least about 97%, at least about 98%, at least about 99%, or about 100% identical to the amino acid sequence of SEQ ID NO: 18.

In some embodiments, the targeting agent, antibody, or binding fragment thereof comprises a VL that comprises an amino acid sequence at least about 90%, at least about 91%, at least about 92%, at least about 93%, at least about 94%, at least about 95%, at least about 96%, at least about 97%, at least about 98%, at least about 99%, or about 100% identical to the amino acid sequence of SEQ ID NO: 20.

In some embodiments, the targeting agent, antibody, or binding fragment thereof comprises a light chain variable region (VL) that comprises an amino acid sequence at least about 90%, at least about 91%, at least about 92%, at least about 93%, at least about 94%, at least about 95%, at least about 96%, at least about 97%, at least about 98%, at least about 99%, or about 100% identical to the amino acid sequence of SEQ ID NO: 21.

In some embodiments, the targeting agent, antibody, or binding fragment thereof comprises a VL that comprises an amino acid sequence at least about 90%, at least about 91%, at least about 92%, at least about 93%, at least about 94%, at least about 95%, at least about 96%, at least about 97%, at least about 98%, at least about 99%, or about 100% identical to the amino acid sequence of SEQ ID NO: 22.

In some embodiments, the targeting agent, antibody, or binding fragment thereof comprises a VH that comprises and/or consists of an amino acid sequence at least about 90%, at least about 91%, at least about 92%, at least about 93%, at least about 94%, at least about 95%, at least about 96%, at least about 97%, at least about 98%, at least about 99%, or about 100% identical to the amino acid sequence of SEQ ID NO: 15, and comprises a VL that comprises and/or consists of an amino acid sequence at least about 90%, at least about 91%, at least about 92%, at least about 93%, at least about 94%, at least about 95%, at least about 96%, at least about 97%, at least about 98%, at least about 99%, or about 100% identical to the amino acid sequence of SEQ ID NO: 19.

In some embodiments, the targeting agent, antibody, or binding fragment thereof comprises a VH that comprises and/or consists of an amino acid sequence that is 100% identical to the amino acid sequence of SEQ ID NO: 15.

In some embodiments, the targeting agent, antibody, or binding fragment thereof comprises a VL that comprises and/or consists of an amino acid sequence that is 100% identical to the amino acid sequence of SEQ ID NO: 19.

Any combination of a VH sequence and a VL sequence shown in Table A is also encompassed by the present disclosure.

TABLE A
anti-CDCP1 antibody heavy chain regions (or heavy chain variable
regions) and antibody light chain regions (or light chain variable regions)

| Ab | SEQ | | SEQ | | |
|---|---|---|---|---|---|
| name: | ID NO | VH | ID NO | VL | Source |
| h12A04 | 23 | QVTLRESGPALVKPTQTLTLT | 159 | DIVMTQSPLSLPVTPGEPA | US20230050380A1 |
| 1- | | CTFSGFSLSTSGMGVSWIRQP | | SISCRSSQSIVHSSGNTYL | |
| VH1/VL | | PGKALEWLAHIYWDDDKRYNP | | EWYLQKPGQSPQLLIYKVS | |
| | | SLKSRLTISKDTSKNQVVLTM | | NRFSGVPDRFSGSGSGTDF | |
| | | TNMDPVDTATYYCARSGSYYG | | TLKISRVEAEDVGVYYCFQ | |
| | | SSLDSWGQGTTVTVSS | | GSHVPWTFGGGTKVEIK | |
| h12A04 | 23 | QVTLRESGPALVKPTQTLTLT | 160 | DIVMTQSPLSLPVTPGEPA | US20230050380A1 |
| 1- | | CTFSGFSLSTSGMGVSWIRQP | | SISCRSSQSIVYSSGNTYL | |
| VH1/VLA | | PGKALEWLAHIYWDDDKRYNP | | EWYLQKPGQSPQLLIYKVS | |
| | | SLKSRLTISKDTSKNQVVLTM | | NRFSGVPDRFSGSGSGTDF | |
| | | TNMDPVDTATYYCARSGSYYG | | TLKISRVEAEDVGVYYCFQ | |
| | | SSLDSWGQGTTVTVSS | | GSHVPWTFGGGTKVEIK | |
| h12A04 | 23 | QVTLRESGPALVKPTQTLTLT | 161 | DIVMTQSPLSLPVTPGEPA | US20230050380A1 |
| 1- | | CTFSGFSLSTSGMGVSWIRQP | | SISCRSSQNIVHSDGNTYL | |
| VH1/VLB | | PGKALEWLAHIYWDDDKRYNP | | EWYLQKPGQSPQLLIYKVS | |
| | | SLKSRLTISKDTSKNQVVLTM | | NRFSGVPDRFSGSGSGTDF | |
| | | TNMDPVDTATYYCARSGSYYG | | TLKISRVEAEDVGVYYCFQ | |
| | | SSLDSWGQGTTVTVSS | | GSHVPWTFGGGTKVEIK | |
| h12A04 | 24 | QVTLRESGPALVKPTQTLTLT | 162 | DIVMTQSPLSLPVTPGEPA | US20230050380A1 |
| 1- | | CTFSGFSLSTFGMGVSWIRQP | | SISCRSSQSIVHSSGNTYL | |
| VH1A/VL | | PGKALEWLAHIYWDDDKRYNP | | EWYLQKPGQSPQLLIYKVS | |
| | | SLKSRLTISKDTSKNQVVLTM | | NRFSGVPDRFSGSGSGTDF | |
| | | TNMDPVDTATYYCARSGSYYG | | TLKISRVEAEDVGVYYCFQ | |
| | | SSLDSWGQGTTVTVSS | | GSHVPWTFGGGTKVEIK | |
| h14A04 | 23 | QVQLVQSGAEVKKPGSSVKVS | 163 | EIVLTQSPATLSLSPGERA | US20230050380A1 |
| 3VH1/VL1 | | CKASGFNIKDYYMNWVRQAPG | | TLSCSASSSVSYLYWYQQK | |
| | | QGLEWIGWIDPENANTIYDPK | | PGQAPRPWIYLTSNLASGV | |
| | | FQGRVTITADESTSTAYMELS | | PARFSGSGSGTDFTLTISS | |
| | | SLRSEDTAVYYCYGSSYRFTY | | LEPEDFAVYYCQQWSSNPF | |
| | | WGQGTLVTVSS | | TFGGGTKVEIK | |
| h14A04 | 23 | QVQLVQSGAEVKKPGSSVKVS | 164 | EIVLTQSPATLSLSPGERA | US20230050380A1 |
| 3VH1/VL2 | | CKASGFNIKDYYMNWVRQAPG | | TLSCSASSSVSYLYWYQQK | |
| | | QGLEWIGWIDPENANTIYDPK | | PGQAPRPWIYLTSNLASGV | |
| | | FQGRVTITADESTSTAYMELS | | PARFSGSGSGTDYTLTISS | |
| | | SLRSEDTAVYYCYGSSYRFTY | | LEPEDFAVYYCQQWSSNPF | |
| | | WGQGTLVTVSS | | TFGGGTKVEIK | |
| h14A04 | 23 | QVQLVQSGAEVKKPGSSVKVS | 165 | EIVLIQSPATLSLSPGERA | US20230050380A1 |
| 3VH1/VL3 | | CKASGFNIKDYYMNWVRQAPG | | TLSCSASSSVSYLYWYQQK | |
| | | QGLEWIGWIDPENANTIYDPK | | PGQAPRPWIYLTSNLASGV | |
| | | FQGRVTITADESTSTAYMELS | | PARFSGSGSGTDFTLTISS | |
| | | SLRSEDTAVYYCYGSSYRFTY | | LEPEDFAVYYCQQWSSNPF | |
| | | WGQGTLVTVSS | | TFGGGTKVEIK | |
| h14A04 | 25 | QVQLVQSGAEVKKPGSSVKVS | 166 | EIVLTQSPATLSLSPGERA | US20230050380A1 |
| 3VH2VL2 | | CKASGFNIKDYYMNWVRQAPG | | TLSCSASSSVSYLYWYQQK | |
| | | QGLEWIGWIDPENANTIYDPK | | PGQAPRPWIYLTSNLASGV | |
| | | FQGRATITADESTSTAYMELS | | PARFSGSGSGTDYTLTISS | |
| | | SLRSEDTAVYYCYGSSYRFTY | | LEPEDFAVYYCQQWSSNPF | |
| | | WGQGTLVTVSS | | TFGGGTKVEIK | |
| h14A04 | 25 | QVQLVQSGAEVKKPGSSVKVS | 167 | EIVLIQSPATLSLSPGERA | US20230050380A1 |
| 3VH2/VL3 | | CKASGFNIKDYYMNWVRQAPG | | TLSCSASSSVSYLYWYQQK | |
| | | QGLEWIGWIDPENANTIYDPK | | PGQAPRPWIYLTSNLASGV | |
| | | FQGRATITADESTSTAYMELS | | PARFSGSGSGTDFTLTISS | |
| | | SLRSEDTAVYYCYGSSYRFTY | | LEPEDFAVYYCQQWSSNPF | |
| | | WGQGTLVTVSS | | TFGGGTKVEIK | |
| h14A06 | 26 | EVQLVESGGGLVQPGGSLRLS | 168 | EIVLTQSPATLSLSPGERA | US20230050380A1 |
| 3VH4/VL1 | | CAASGFTFSSYTMSWVRQAPE | | TLSCRASESVDSYGNSFMH | |
| | | KRLEWVAYISSGGGSTYYPDT | | WYQQKPGQAPRLLIFLASN | |
| | | VKGRFTISRDNAKNSLYLQMN | | LESGVPARFSGSGSRTDFT | |
| | | SLRAEDTAVYYCARYDLGFAY | | LTISSLEPEDFAVYYCQQN | |
| | | WGQGTLVTVSS | | NEDPWTFGGGTKVEIK | |
| h14A06 | 26 | EVQLVESGGGLVQPGGSLRLS | 169 | EIVLTQSPATLSLSPGERA | US20230050380A1 |
| 3VH4/VL2 | | CAASGFTFSSYTMSWVRQAPE | | TLSCRASESVDSYGNSFMH | |
| | | KRLEWVAYISSGGGSTYYPDT | | WYQQKPGQAPRLLIFLASN | |
| | | VKGRFTISRDNAKNSLYLQMN | | LESGVPARFSGSGSGTDFT | |
| | | SLRAEDTAVYYCARYDLGFAY | | LTISSLEPEDFAVYYCQQN | |
| | | WGQGTLVTVSS | | NEDPWTFGGGTKVEIK | |
| h12A04 | 27 | QVTLRESGPALVKPTQTLTLT | | | US20230050380A1 |
| 1VH2 | | CTFSGFSLSTSGMGVSWIRQP | | | |
| | | PGKGLEWLAHIYWDDDKRYNP | | | |
| | | SLKSRLTISKDTSKNQVVLTM | | | |
| | | TNMDPVDTATYYCARSGSYYG | | | |
| | | SSLDSWGQGTTVTVSS | | | |
| h12A04 | 28 | QVTLRESGPALVKPTQTLTLT | | | US20230050380A1 |
| 1VH2A | | CTFSGFSLSTFGMGVSWIRQP | | | |
| | | PGKGLEWLAHIYWDDDKRYNP | | | |
| | | SLKSRLTISKDTSKNQVVLTM | | | |
| | | TNMDPVDTATYYCARSGSYYG | | | |
| | | SSLDSWGQGTTVTVSS | | | |
| h14A06 | 29 | EVQLVESGGGLVQPGGSLRLS | | | US20230050380A1 |
| 3VH2 | | CAASGFTFSSYTMSWVRQAPG | | | |
| | | KRLEWVAYISSGGGSTYYPDT | | | |
| | | VKGRFTISRDNAKNSLYLQMN | | | |
| | | SLRAEDTAVYYCARYDLGFAY | | | |
| | | WGQGTLVTVSS | | | |
| h14A06 | 30 | EVQLQESGGGLVQPGGSLRLS | | | US20230050380A1 |
| 3VH3 | | CAASGFTFSSYTMSWVRQAPG | | | |
| | | KRLEWVAYISSGGGSTYYPDT | | | |
| | | VKGRFTISRDNAKNSLYLQMN | | | |
| | | SLRAEDTAVYYCARYDLGFAY | | | |
| | | WGQGTLVTVSS | | | |
| CL03 | 31 | EVQLVESGGGLVQPGGSLRLS | 170 | DIQMTQSPSSLSASVGDRV | WO2022212876 |
| Fab | | CAASGFNFSSYSIHWVRQAPG | | TITCRASQSVSSAVAWYQQ | |
| | | KGLEWVASIYPYSGSTYYADS | | KPGKAPKLLIYSASSLYSG | |
| | | VKGRFTISADTSKNTAYLQMN | | VPSRFSGSRSGTDFTLTIS | |
| | | SLRAEDTAVYYCARQSHSYYS | | SLQPEDFATYYCQQSGQRP | |
| | | HTWWVSYGMDYWGQGTLVTVS | | ITFGQGTKVEIK | |
| | | S | | | |
| CL03 | 32 | EVQLVESGGGLVQPGGSLRLS | 170 | DIQMTQSPSSLSASVGDRV | WO2022212876 |
| IgG | | CAASGFNFSSYSIHWVRQAPG | | TITCRASQSVSSAVAWYQQ | |
| | | KGLEWVASIYPYSGSTYYADS | | KPGKAPKLLIYSASSLYSG | |
| | | VKGRFTISADTSKNTAYLQMN | | VPSRFSGSRSGTDFTLTIS | |
| | | SLRAEDTAVYYCARQSHSYYS | | SLQPEDFATYYCQQSGQRP | |
| | | HTWWVSYGMDYWGQGTLVTVS | | ITFGQGTKVEIK | |
| | | S | | | |
| CL03 | 33 | EVQLVESGGGLVQPGGSLRLS | 170 | DIQMTQSPSSLSASVGDRV | WO2022212876 |
| IgG | | CAASGFDFSSYSIHWVRQAPG | | TITCRASQSVSSAVAWYQQ | |
| H1- | | KGLEWVASIYPYSGSTYYADS | | KPGKAPKLLIYSASSLYSG | |
| NtoD | | VKGRFTISADTSKNTAYLQMN | | VPSRFSGSRSGTDFTLTIS | |
| | | SLRAEDTAVYYCARQSHSYYS | | SLQPEDFATYYCQQSGQRP | |
| | | HTWWVSYGMDYWGQGTLVTVS | | ITFGQGTKVEIK | |
| | | S | | | |
| CL03 | 34 | EVQLVESGGGLVQPGGSLRLS | 170 | DIQMTQSPSSLSASVGDRV | WO2022212876 |
| Fab | | CAASGFDFSSYSIHWVRQAPG | | TITCRASQSVSSAVAWYQQ | |
| | | KGLEWVASIYPYSGSTYYADS | | KPGKAPKLLIYSASSLYSG | |
| | | VKGRFTISADTSKNTAYLQMN | | VPSRFSGSRSGTDFTLTIS | |
| | | SLRAEDTAVYYCARQSHSYYS | | SLQPEDFATYYCQQSGQRP | |
| | | HTWWVSYGADYWGQGTLVTVS | | ITFGQGTKVEIK | |
| | | S | | | |
| CL03 | 35 | EVQLVESGGGLVQPGGSLRLS | 170 | DIQMTQSPSSLSASVGDRV | WO2022212876 |
| IqG | | CAASGFDESSYSIHWVRQAPG | | TITCRASQSVSSAVAWYQQ | |
| H1- | | KGLEWVASIYPYSGSTYYADS | | KPGKAPKLLIYSASSLYSG | |
| NtoD | | VKGRFTISADTSKNTAYLQMN | | VPSRFSGSRSGTDFTLTIS | |
| H3- | | SLRAEDTAVYYCARQSHSYYS | | SLQPEDFATYYCQQSGQRP | |
| MtoA | | HTWWVSYGIDYWGQGTLVTVS | | ITFGQGTKVEIK | |
| (″IgG101″) | | S | | | |
| CL03 | 36 | EVQLVESGGGLVQPGGSLRLS | 170 | DIQMTQSPSSLSASVGDRV | WO2022212876 |
| IgG | | CAASGFDFSSYSIHWVRQAPG | | TITCRASQSVSSAVAWYQQ | |
| H1- | | KGLEWVASIYPYSGSTYYADS | | KPGKAPKLLIYSASSLYSG | |
| NtoD | | VKGRFTISADTSKNTAYLQMN | | VPSRFSGSRSGTDFTLTIS | |
| H3- | | SLRAEDTAVYYCARQSHSYYS | | SLQPEDFATYYCQQSGQRP | |
| Mtol | | HTWWVSYGLDYWGQGTLVTVS | | ITFGQGTKVEIK | |
| | | S | | | |
| CL03 | 37 | EVQLVESGGGLVQPGGSLRLS | 170 | DIQMTQSPSSLSASVGDRV | WO2022212876 |
| IqG | | CAASGFDFSSYSIHWVRQAPG | | TITCRASQSVSSAVAWYQQ | |
| H1- | | KGLEWVASIYPYSGSTYYADS | | KPGKAPKLLIYSASSLYSG | |
| NtoD | | VKGRFTISADTSKNTAYLQMN | | VPSRFSGSRSGTDFTLTIS | |
| H3- | | SLRAEDTAVYYCARQSHSYYS | | SLQPEDFATYYCQQSGQRP | |
| MtoL | | HTWWVSYGVDYWGQGTLVTVS | | ITFGQGTKVEIK | |
| | | S | | | |
| CL03 | 38 | EVQLVESGGGLVQPGGSLRLS | 171 | DIQMTQSPSSLSASVGDRV | WO2022212876 |
| IqG | | CAASGENFSSSSIHWVRQAPG | | TITCRASQSVSSAVAWYQQ | |
| H1- | | KGLEWVASIYPYSGSTSYADS | | KPGKAPKLLIYSASSLYSG | |
| NtoD | | VKGRFTISADTSKNTAYLQMN | | VPSRFSGSRSGTDFTLTIS | |
| H3- | | SLRAEDTAVYYCARTYWVQFW | | SLQPEDFATYYCQQSSSSL | |
| MtoV | | YGHFSYYRPALDYWGQGTLVT | | ITFGQGTKVEIK | |
| | | VSS | | | |
| CL07 | 38 | EVQLVESGGGLVQPGGSLRLS | 171 | DIQMTQSPSSLSASVGDRV | WO2022212876 |
| Fab | | CAASGFNFSSSSIHWVRQAPG | | TITCRASQSVSSAVAWYQQ | |
| | | KGLEWVASIYPYSGSTSYADS | | KPGKAPKLLIYSASSLYSG | |
| | | VKGRFTISADTSKNTAYLQMN | | VPSRFSGSRSGTDFTLTIS | |
| | | SLRAEDTAVYYCARTYWVQFW | | SLQPEDFATYYCQQSSSSL | |
| | | YGHFSYYRPALDYWGQGTLVT | | ITFGQGTKVEIK | |
| | | VSS | | | |
| CL07 | 39 | EVQLVESGGGLVQPGGSLRLS | 171 | DIQMTQSPSSLSASVGDRV | WO2022212876 |
| IgG | | CAASGFDFSSSSIHWVRQAPG | | TITCRASQSVSSAVAWYQQ | |
| | | KGLEWVASIYPYSGSTSYADS | | KPGKAPKLLIYSASSLYSG | |
| | | VKGRFTISADTSKNTAYLQMN | | VPSRFSGSRSGTDFTLTIS | |
| | | SLRAEDTAVYYCARTYWVQFW | | SLQPEDFATYYCQQSSSSL | |
| | | YGHFSYYRPALDYWGQGTLVT | | ITFGQGTKVEIK | |
| | | VSS | | | |
| CL07 | 33 | EVQLVESGGGLVQPGGSLRLS | 171 | DIQMTQSPSSLSASVGDRV | WO2022212876 |
| IgG | | CAASGFDFSSYSIHWVRQAPG | | TITCRASQSVSSAVAWYQQ | |
| H1- | | KGLEWVASIYPYSGSTYYADS | | KPGKAPKLLIYSASSLYSG | |
| NtoD | | VKGRFTISADTSKNTAYLQMN | | VPSRFSGSRSGTDFTLTIS | |
| | | SLRAEDTAVYYCARQSHSYYS | | SLQPEDFATYYCQQSSSSL | |
| | | HTWWVSYGMDYWGQGTLVTVS | | ITFGQGTKVEIK | |
| | | S | | | |
| IgG3H7L | 40 | EVQLVESGGGLVQPGGSLRLS | 170 | DIQMTQSPSSLSASVGDRV | WO2022212876 |
| | | CAASGFDFSSYSIHWVRQAPG | | TITCRASQSVSSAVAWYQQ | |
| | | KGLEWVASIYPYSGSTYYADS | | KPGKAPKLLIYSASSLYSG | |
| | | VKGRFTISADTSKNTAYLQMN | | VPSRFSGSRSGTDFTLTIS | |
| | | SLRAEDTAVYYCARQNHSYYS | | SLQPEDFATYYCQQSGQRP | |
| | | HTWWVSYGMDYWGQGTLVTVS | | ITFGQGTKVEIK | |
| | | S | | | |
| IgGA01 | 41 | EVQLVESGGGLVQPGGSLRLS | 170 | DIQMTQSPSSLSASVGDRV | WO2022212876 |
| | | CAASGFDESSYSIHWVRQAPG | | TITCRASQSVSSAVAWYQQ | |
| | | KGLEWVASIYPYSGSTYYADS | | KPGKAPKLLIYSASSLYSG | |
| | | VKGRFTISADTSKNTAYLQMN | | VPSRFSGSRSGTDFTLTIS | |
| | | SLRAEDTAVYYCARQTHSYYS | | SLQPEDFATYYCQQSGQRP | |
| | | HTWWVSYGMDYWGQGTLVTVS | | ITFGQGTKVEIK | |
| | | S | | | |
| IgGA03 | 42 | EVQLVESGGGLVQPGGSLRLS | 170 | DIQMTQSPSSLSASVGDRV | WO2022212876 |
| | | CAASGFDFSSYSIHWVRQAPG | | TITCRASQSVSSAVAWYQQ | |
| | | KGLEWVASIYPYSGSTYYADS | | KPGKAPKLLIYSASSLYSG | |
| | | VKGRFTISADTSKNTAYLQMN | | VPSRFSGSRSGTDFTLTIS | |
| | | SLRAEDTAVYYCARQGHSYYS | | SLQPEDFATYYCQQSGQRP | |
| | | HTWWVSYGMDYWGQGTLVTVS | | ITFGQGTKVEIK | |
| | | S | | | |
| IgGA04 | 43 | EVQLVESGGGLVQPGGSLRLS | 170 | DIQMTQSPSSLSASVGDRV | WO2022212876 |
| | | CAASGFDFSSYSIHWVRQAPG | | TITCRASQSVSSAVAWYQQ | |
| | | KGLEWVASIYPYSGSTYYADS | | KPGKAPKLLIYSASSLYSG | |
| | | VKGRFTISADTSKNTAYLQMN | | VPSRFSGSRSGTDFTLTIS | |
| | | SLRAEDTAVYYCARQAHSYYS | | SLQPEDFATYYCQQSGQRP | |
| | | HTWWVSYGMDYWGQGTLVTVS | | ITFGQGTKVEIK | |
| | | S | | | |
| IgGA05 | 44 | EVQLVESGGGLVQPGGSLRLS | 170 | DIQMTQSPSSLSASVGDRV | WO2022212876 |
| | | CAASGFDFSSYSIHWVRQAPG | | TITCRASQSVSSAVAWYQQ | |
| | | KGLEWVASIYPYSGSTYYADS | | KPGKAPKLLIYSASSLYSG | |
| | | VKGRFTISADTSKNTAYLQMN | | VPSRFSGSRSGTDFTLTIS | |
| | | SLRAEDTAVYYCARQGQSYYS | | SLQPEDFATYYCQQSGQRP | |
| | | HTWWVSYGMDYWGQGTLVTVS | | ITFGQGTKVEIK | |
| | | S | | | |
| IgGA06 | 33 | EVQLVESGGGLVQPGGSLRLS | 172 | DIQMTQSPSSLSASVGDRV | WO2022212876 |
| | | CAASGFDFSSYSIHWVRQAPG | | TITCRASQSVSSAVAWYQQ | |
| | | KGLEWVASIYPYSGSTYYADS | | KPGKAPKLLIYSASSLYSG | |
| | | VKGRFTISADTSKNTAYLQMN | | VPSRFSGSRSGTDFTLTIS | |
| | | SLRAEDTAVYYCARQSHSYYS | | SLQPEDFATYYCQQSGERP | |
| | | HTWWVSYGMDYWGQGTLVTVS | | ITFGQGTKVEIK | |
| | | S | | | |
| IgGA08 | 33 | EVQLVESGGGLVQPGGSLRLS | 173 | DIQMTQSPSSLSASVGDRV | WO2022212876 |
| | | CAASGFDFSSYSIHWVRQAPG | | TITCRASQSVSSAVAWYQQ | |
| | | KGLEWVASIYPYSGSTYYADS | | KPGKAPKLLIYSASSLYSG | |
| | | VKGRFTISADTSKNTAYLQMN | | VPSRFSGSRSGTDFTLTIS | |
| | | SLRAEDTAVYYCARQSHSYYS | | SLQPEDFATYYCQQSGNVP | |
| | | HTWWVSYGMDYWGQGTLVTVS | | ATFGQGTKVEIK | |
| | | S | | | |
| IgGA09 | 33 | EVQLVESGGGLVQPGGSLRLS | 174 | DIQMTQSPSSLSASVGDRV | WO2022212876 |
| | | CAASGFDFSSYSIHWVRQAPG | | TITCRASQSVSSAVAWYQQ | |
| | | KGLEWVASIYPYSGSTYYADS | | KPGKAPKLLIYSASSLYSG | |
| | | VKGRFTISADTSKNTAYLQMN | | VPSRFSGSRSGTDFTLTIS | |
| | | SLRAEDTAVYYCARQSHSYYS | | SLQPEDFATYYCQQSGKRP | |
| | | HTWWVSYGMDYWGQGTLVTVS | | MTFGQGTKVEIK | |
| | | S | | | |
| IgGA10 | 45 | EVQLVESGGGLVQPGGSLRLS | 175 | DIQMTQSPSSLSASVGDRV | WO2022212876 |
| | | CAASGFDFSSYSIHWVRQAPG | | TITCRASQSVSSAVAWYQQ | |
| | | KGLEWVASIYPYSGSTYYADS | | KPGKAPKLLIYSASSLYSG | |
| | | VKGRFTISADTSKNTAYLQMN | | VPSRFSGSRSGTDFTLTIS | |
| | | SLRAEDTAVYYCARRSHSYYS | | SLQPEDFATYYCQQTGQRP | |
| | | HTWWVSYGMDYWGQGTLVTVS | | MTFGQGTKVEIK | |
| | | S | | | |
| IgGA11 | 33 | EVQLVESGGGLVQPGGSLRLS | 176 | DIQMTQSPSSLSASVGDRV | WO2022212876 |
| | | CAASGFDFSSYSIHWVRQAPG | | TITCRASQSVSSAVAWYQQ | |
| | | KGLEWVASIYPYSGSTYYADS | | KPGKAPKLLIYSASSLYSG | |
| | | VKGRFTISADTSKNTAYLQMN | | VPSRFSGSRSGTDFTLTIS | |
| | | SLRAEDTAVYYCARQSHSYYS | | SLQPEDFATYYCQQSMQWP | |
| | | HTWWVSYGMDYWGQGTLVTVS | | KTFGQGTKVEIK | |
| | | S | | | |
| IgGB01 | 46 | EVQLVESGGGLVQPGGSLRLS | 177 | DIQMTQSPSSLSASVGDRV | WO2022212876 |
| | | CAASGFDFSSYSIHWVRQAPG | | TITCRASQSVSSAVAWYQQ | |
| | | KGLEWVASIYPYSGSTYYADS | | KPGKAPKLLIYSASSLYSG | |
| | | VKGRFTISADTSKNTAYLQMN | | VPSRFSGSRSGTDFTLTIS | |
| | | SLRAEDTAVYYCARQSHSFYS | | SLQPEDFATYYCQQSLQRP | |
| | | HTWWVSYGMDYWGQGTLVTVS | | MTFGQGTKVEIK | |
| | | S | | | |
| IgGB03 | 45 | EVQLVESGGGLVQPGGSLRLS | 178 | DIQMTQSPSSLSASVGDRV | WO2022212876 |
| | | CAASGFDFSSYSIHWVRQAPG | | TITCRASQSVSSAVAWYQQ | |
| | | KGLEWVASIYPYSGSTYYADS | | KPGKAPKLLIYSASSLYSG | |
| | | VKGRFTISADTSKNTAYLQMN | | VPSRFSGSRSGTDFTLTIS | |
| | | SLRAEDTAVYYCARRSHSYYS | | SLQPEDFATYYCQQVELVP | |
| | | HTWWVSYGMDYWGQGTLVTVS | | MTFGQGTKVEIK | |
| | | S | | | |
| IgGB04 | 47 | EVQLVESGGGLVQPGGSLRLS | 179 | DIQMTQSPSSLSASVGDRV | WO2022212876 |
| | | CAASGFDFSSYSIHWVRQAPG | | TITCRASQSVSSAVAWYQQ | |
| | | KGLEWVASIYPYSGSTYYADS | | KPGKAPKLLIYSASSLYSG | |
| | | VKGRFTISADTSKNTAYLQMN | | VPSRFSGSRSGTDFTLTIS | |
| | | SLRAEDTAVYYCARQDHSYYS | | SLQPEDFATYYCQQAGKRP | |
| | | HTWWVSYGMDYWGQGTLVTVS | | LTFGQGTKVEIK | |
| | | S | | | |
| IgGB07 | 45 | EVQLVESGGGLVQPGGSLRLS | 180 | DIQMTQSPSSLSASVGDRV | WO2022212876 |
| | | CAASGFDFSSYSIHWVRQAPG | | TITCRASQSVSSAVAWYQQ | |
| | | KGLEWVASIYPYSGSTYYADS | | KPGKAPKLLIYSASSLYSG | |
| | | VKGRFTISADTSKNTAYLQMN | | VPSRFSGSRSGTDFTLTIS | |
| | | SLRAEDTAVYYCARRSHSYYS | | SLQPEDFATYYCQQSVDLT | |
| | | HTWWVSYGMDYWGQGTLVTVS | | ATFGQGTKVEIK | |
| | | S | | | |
| IgGB08 | 45 | EVQLVESGGGLVQPGGSLRLS | 181 | DIQMTQSPSSLSASVGDRV | WO2022212876 |
| | | CAASGFDFSSYSIHWVRQAPG | | TITCRASQSVSSAVAWYQQ | |
| | | KGLEWVASIYPYSGSTYYADS | | KPGKAPKLLIYSASSLYSG | |
| | | VKGRFTISADTSKNTAYLQMN | | VPSRFSGSRSGTDFTLTIS | |
| | | SLRAEDTAVYYCARRSHSYYS | | SLQPEDFATYYCQQLGVRA | |
| | | HTWWVSYGMDYWGQGTLVTVS | | ATFGQGTKVEIK | |
| | | S | | | |
| IgGC01 | 33 | EVQLVESGGGLVQPGGSLRLS | 182 | DIQMTQSPSSLSASVGDRV | WO2022212876 |
| | | CAASGFDFSSYSIHWVRQAPG | | TITCRASQSVSSAVAWYQQ | |
| | | KGLEWVASIYPYSGSTYYADS | | KPGKAPKLLIYSASSLYSG | |
| | | VKGRFTISADTSKNTAYLQMN | | VPSRFSGSRSGTDFTLTIS | |
| | | SLRAEDTAVYYCARQSHSYYS | | SLQPEDFATYYCQQSGRKP | |
| | | HTWWVSYGMDYWGQGTLVTVS | | VTFGQGTKVEIK | |
| | | S | | | |
| IgGC03 | 45 | EVQLVESGGGLVQPGGSLRLS | 183 | DIQMTQSPSSLSASVGDRV | WO2022212876 |
| | | CAASGFDFSSYSIHWVRQAPG | | TITCRASQSVSSAVAWYQQ | |
| | | KGLEWVASIYPYSGSTYYADS | | KPGKAPKLLIYSASSLYSG | |
| | | VKGRFTISADTSKNTAYLQMN | | VPSRFSGSRSGTDFTLTIS | |
| | | SLRAEDTAVYYCARRSHSYYS | | SLQPEDFATYYCQQTAQSP | |
| | | HTWWVSYGMDYWGQGTLVTVS | | LTFGQGTKVEIK | |
| | | S | | | |
| IgGC04 | 33 | EVQLVESGGGLVQPGGSLRLS | 184 | DIQMTQSPSSLSASVGDRV | WO2022212876 |
| | | CAASGFDFSSYSIHWVRQAPG | | TITCRASQSVSSAVAWYQQ | |
| | | KGLEWVASIYPYSGSTYYADS | | KPGKAPKLLIYSASSLYSG | |
| | | VKGRFTISADTSKNTAYLQMN | | VPSRFSGSRSGTDFTLTIS | |
| | | SLRAEDTAVYYCARQSHSYYS | | SLQPEDFATYYCQQSRQRP | |
| | | HTWWVSYGMDYWGQGTLVTVS | | LTFGQGTKVEIK | |
| | | S | | | |
| IgGC05 | 33 | EVQLVESGGGLVQPGGSLRLS | 185 | DIQMTQSPSSLSASVGDRV | WO2022212876 |
| | | CAASGFDFSSYSIHWVRQAPG | | TITCRASQSVSSAVAWYQQ | |
| | | KGLEWVASIYPYSGSTYYADS | | KPGKAPKLLIYSASSLYSG | |
| | | VKGRFTISADTSKNTAYLQMN | | VPSRFSGSRSGTDFTLTIS | |
| | | SLRAEDTAVYYCARQSHSYYS | | SLQPEDFATYYCQQSGLMP | |
| | | HTWWVSYGMDYWGQGTLVTVS | | LTFGQGTKVEIK | |
| | | S | | | |
| IgGC06 | 33 | EVQLVESGGGLVQPGGSLRLS | 186 | DIQMTQSPSSLSASVGDRV | WO2022212876 |
| | | CAASGFDFSSYSIHWVRQAPG | | TITCRASQSVSSAVAWYQQ | |
| | | KGLEWVASIYPYSGSTYYADS | | KPGKAPKLLIYSASSLYSG | |
| | | VKGRFTISADTSKNTAYLQMN | | VPSRFSGSRSGTDFTLTIS | |
| | | SLRAEDTAVYYCARQSHSYYS | | SLQPEDFATYYCQQSGHRP | |
| | | HTWWVSYGMDYWGQGTLVTVS | | ITFGQGTKVEIK | |
| | | S | | | |
| IgGC09 | 48 | EVQLVESGGGLVQPGGSLRLS | 187 | DIQMTQSPSSLSASVGDRV | WO2022212876 |
| | | CAASGFDFSSYSIHWVRQAPG | | TITCRASQSVSSAVAWYQQ | |
| | | KGLEWVASIYPYSGSTYYADS | | KPGKAPKLLIYSASSLYSG | |
| | | VKGRFTISADTSKNTAYLQMN | | VPSRFSGSRSGTDFTLTIS | |
| | | SLRAEDTAVYYCARKYHSYYS | | SLQPEDFATYYCQQSSQRP | |
| | | HTWWVSYGMDYWGQGTLVTVS | | ATFGQGTKVEIK | |
| | | S | | | |
| IgGD02 | 47 | EVQLVESGGGLVQPGGSLRLS | 188 | DIQMTQSPSSLSASVGDRV | WO2022212876 |
| | | CAASGFDFSSYSIHWVRQAPG | | TITCRASQSVSSAVAWYQQ | |
| | | KGLEWVASIYPYSGSTYYADS | | KPGKAPKLLIYSASSLYSG | |
| | | VKGRFTISADTSKNTAYLQMN | | VPSRFSGSRSGTDFTLTIS | |
| | | SLRAEDTAVYYCARQDHSYYS | | SLQPEDFATYYCQQSGRQP | |
| | | HTWWVSYGMDYWGQGTLVTVS | | LTFGQGTKVEIK | |
| | | S | | | |
| IgGD03 | 45 | EVQLVESGGGLVQPGGSLRLS | 189 | DIQMTQSPSSLSASVGDRV | WO2022212876 |
| | | CAASGFDFSSYSIHWVRQAPG | | TITCRASQSVSSAVAWYQQ | |
| | | KGLEWVASIYPYSGSTYYADS | | KPGKAPKLLIYSASSLYSG | |
| | | VKGRFTISADTSKNTAYLQMN | | VPSRFSGSRSGTDFTLTIS | |
| | | SLRAEDTAVYYCARRSHSYYS | | SLQPEDFATYYCQQFMRPA | |
| | | HTWWVSYGMDYWGQGTLVTVS | | FTFGQGTKVEIK | |
| | | S | | | |
| IgGD04 | 45 | EVQLVESGGGLVQPGGSLRLS | 190 | DIQMTQSPSSLSASVGDRV | WO2022212876 |
| | | CAASGFDFSSYSIHWVRQAPG | | TITCRASQSVSSAVAWYQQ | |
| | | KGLEWVASIYPYSGSTYYADS | | KPGKAPKLLIYSASSLYSG | |
| | | VKGRFTISADTSKNTAYLQMN | | VPSRFSGSRSGTDFTLTIS | |
| | | SLRAEDTAVYYCARRSHSYYS | | SLQPEDFATYYCQQSGQPS | |
| | | HTWWVSYGMDYWGQGTLVTVS | | MTFGQGTKVEIK | |
| | | S | | | |
| IgGD07 | 33 | EVQLVESGGGLVQPGGSLRLS | 191 | DIQMTQSPSSLSASVGDRV | WO2022212876 |
| | | CAASGFDFSSYSIHWVRQAPG | | TITCRASQSVSSAVAWYQQ | |
| | | KGLEWVASIYPYSGSTYYADS | | KPGKAPKLLIYSASSLYSG | |
| | | VKGRFTISADTSKNTAYLQMN | | VPSRFSGSRSGTDFTLTIS | |
| | | SLRAEDTAVYYCARQSHSYYS | | SLQPEDFATYYCQQSVLRP | |
| | | HTWWVSYGMDYWGQGTLVTVS | | FTFGQGTKVEIK | |
| | | S | | | |
| IgGD08 | 49 | EVQLVESGGGLVQPGGSLRLS | 171 | DIQMTQSPSSLSASVGDRV | WO2022212876 |
| | | CAASGFNFSSSSIHWVRQAPG | | TITCRASQSVSSAVAWYQQ | |
| | | KGLEWVASIYPYSGSTSYADS | | KPGKAPKLLIYSASSLYSG | |
| | | VKGRFTISADTSKNTAYLQMN | | VPSRFSGSRSGTDFTLTIS | |
| | | SLRAEDTAVYYCARIYWVDFW | | SLQPEDFATYYCQQSSSSL | |
| | | YGHFSYYRPALDYWGQGTLVT | | ITFGQGTKVEIK | |
| | | VSS | | | |
| IgGD09 | 50 | EVQLVESGGGLVQPGGSLRLS | 170 | DIQMTQSPSSLSASVGDRV | WO2022212876 |
| | | CAASGFDASSYSIHWVRQAPG | | TITCRASQSVSSAVAWYQQ | |
| | | KGLEWVASIYPYSGSTYYADS | | KPGKAPKLLIYSASSLYSG | |
| | | VKGRFTISADTSKNTAYLQMN | | VPSRFSGSRSGTDFTLTIS | |
| | | SLRAEDTAVYYCARQSHSYYS | | SLQPEDFATYYCQQSGQRP | |
| | | HTWWVSYGMDYWGQGTLVTVS | | ITFGQGTKVEIK | |
| | | S | | | |
| IgG87 | 51 | EVQLVESGGGLVQPGGSLRLS | 170 | DIQMTQSPSSLSASVGDRV | WO2022212876 |
| | | CAASGFDFSSYSAHWVRQAPG | | TITCRASQSVSSAVAWYQQ | |
| | | KGLEWVASIYPYSGSTYYADS | | KPGKAPKLLIYSASSLYSG | |
| | | VKGRFTISADTSKNTAYLQMN | | VPSRFSGSRSGTDFTLTIS | |
| | | SLRAEDTAVYYCARQSHSYYS | | SLQPEDFATYYCQQSGQRP | |
| | | HTWWVSYGMDYWGQGTLVTVS | | ITFGQGTKVEIK | |
| | | S | | | |
| IgG89 | 52 | EVQLVESGGGLVQPGGSLRLS | 170 | DIQMTQSPSSLSASVGDRV | WO2022212876 |
| | | CAASGFDFSSYSIHWVRQAPG | | TITCRASQSVSSAVAWYQQ | |
| | | KGLEWVASIYPYSGSTSYADS | | KPGKAPKLLIYSASSLYSG | |
| | | VKGRFTISADTSKNTAYLQMN | | VPSRFSGSRSGTDFTLTIS | |
| | | SLRAEDTAVYYCARQSHSYYS | | SLQPEDFATYYCQQSGQRP | |
| | | HTWWVSYGMDYWGQGTLVTVS | | ITFGQGTKVEIK | |
| | | S | | | |
| IgG94 | 53 | EVQLVESGGGLVQPGGSLRLS | 170 | DIQMTQSPSSLSASVGDRV | WO2022212876 |
| | | CAASGFDESSYSIHWVRQAPG | | TITCRASQSVSSAVAWYQQ | |
| | | KGLEWVASIYPYSGSTYYADS | | KPGKAPKLLIYSASSLYSG | |
| | | VKGRFTISADTSKNTAYLQMN | | VPSRFSGSRSGTDFTLTIS | |
| | | SLRAEDTAVYYCARQSHSYYS | | SLQPEDFATYYCQQSGQRP | |
| | | HTAWVSYGMDYWGQGTLVTVS | | ITFGQGTKVEIK | |
| | | S | | | |
| IgG97 | 54 | EVQLVESGGGLVQPGGSLRLS | 170 | DIQMTQSPSSLSASVGDRV | WO2022212876 |
| | | CAASGFDFSSYSIHWVRQAPG | | TITCRASQSVSSAVAWYQQ | |
| | | KGLEWVASSYPYSGSTYYADS | | KPGKAPKLLIYSASSLYSG | |
| | | VKGRFTISADTSKNTAYLQMN | | VPSRFSGSRSGTDFTLTIS | |
| | | SLRAEDTAVYYCARQSHSYYS | | SLQPEDFATYYCQQSGQRP | |
| | | HTWWVSYGMDYWGQGTLVTVS | | ITFGQGTKVEIK | |
| | | S | | | |
| CL03 | 55 | EISEVQLVESGGGLVQPGGSL | 192 | MASDIQMTQSPSSLSASVG | WO2022212876 |
| Fab | | RLSCAASGFNFSSYSIHWVRQ | | DRVTITCRASQSVSSAVAW | |
| | | APGKGLEWVASIYPYSGSTYY | | YQQKPGKAPKLLIYSASSL | |
| | | ADSVKGRFTISADTSKNTAYL | | YSGVPSRFSGSRSGTDFTL | |
| | | QMNSLRAEDTAVYYCARQSHS | | TISSLQPEDFATYYCQQSG | |
| | | YYSHTWWVSYGMDYWGQGTLV | | QRPITFGQGTKVEIKRTVA | |
| | | TVSSASTKGPSVFPLAPSSKS | | APSVFIFPPSDSQLKSGTA | |
| | | TSGGTAALGCLVKDYFPEPVT | | SVVCLLNNFYPREAKVQWK | |
| | | VSWNSGALTSGVHTFPAVLQS | | VDNALQSGNSQESVTEQDS | |
| | | SGLYSLSSVVTVPSSSLGTQT | | KDSTYSLSSTLTLSKADYE | |
| | | YICNVNHKPSNTKVDKKVEPK | | KHKVYACEVTHQGLSSPVT | |
| | | SC | | KSFNRGEC | |
| CL03 | 56 | EVQLVESGGGLVQPGGSLRLS | 193 | DIQMTQSPSSLSASVGDRV | WO2022212876 |
| IgG | | CAASGFNFSSYSIHWVRQAPG | | TITCRASQSVSSAVAWYQQ | |
| CL03 | | KGLEWVASIYPYSGSTYYADS | | KPGKAPKLLIYSASSLYSG | |
| | | VKGRFTISADTSKNTAYLQMN | | VPSRFSGSRSGTDFTLTIS | |
| | | SLRAEDTAVYYCARQSHSYYS | | SLQPEDFATYYCQQSGQRP | |
| | | HTWWVSYGMDYWGQGTLVTVS | | ITFGQGTKVEIKRTVAAPS | |
| | | SASTKGPSVFPLAPSSKSTSG | | VFIFPPSDSQLKSGTASVV | |
| | | GTAALGCLVKDYFPEPVTVSW | | CLLNNFYPREAKVQWKVDN | |
| | | NSGALTSGVHTFPAVLQSSGL | | ALQSGNSQESVTEQDSKDS | |
| | | YSLSSVVTVPSSSLGTQTYIC | | TYSLSSTLTLSKADYEKHK | |
| | | NVNHKPSNTKVDKKVEPKSCD | | VYACEVTHQGLSSPVTKSF | |
| | | KTHTCPPCPAPELLGGPSVFL | | NRGEC | |
| | | FPPKPKDTLMISRTPEVTCVV | | | |
| | | VDVSHEDPEVKFNWYVDGVEV | | | |
| | | HNAKTKPREEQYNSTYRVVSV | | | |
| | | LTVLHQDWLNGKEYKCKVSNK | | | |
| | | ALPAPIEKTISKAKGQPREPQ | | | |
| | | VYTLPPSRDELTKNQVSLTCL | | | |
| | | VKGFYPSDIAVEWESNGQPEN | | | |
| | | NYKTTPPVLDSDGSFFLYSKL | | | |
| | | TVDKSRWQQGNVFSCSVMHEA | | | |
| | | LHNHYTQKSLSLSPGK | | | |
| IqG | 57 | EVQLVESGGGLVQPGGSLRLS | 194 | DIQMTQSPSSLSASVGDRV | WO2022212876 |
| H1- | | CAASGFDFSSYSIHWVRQAPG | | TITCRASQSVSSAVAWYQQ | |
| NtoD | | KGLEWVASIYPYSGSTYYADS | | KPGKAPKLLIYSASSLYSG | |
| | | VKGRFTISADTSKNTAYLQMN | | VPSRFSGSRSGTDFTLTIS | |
| | | SLRAEDTAVYYCARQSHSYYS | | SLQPEDFATYYCQQSGQRP | |
| | | HTWWVSYGMDYWGQGTLVTVS | | ITFGQGTKVEIKRTVAAPS | |
| | | SASTKGPSVFPLAPSSKSTSG | | VFIFPPSDSQLKSGTASVV | |
| | | GTAALGCLVKDYFPEPVTVSW | | CLLNNFYPREAKVQWKVDN | |
| | | NSGALTSGVHTFPAVLQSSGL | | ALQSGNSQESVTEQDSKDS | |
| | | YSLSSVVTVPSSSLGTQTYIC | | TYSLSSTLTLSKADYEKHK | |
| | | NVNHKPSNTKVDKKVEPKSCD | | VYACEVTHQGLSSPVTKSF | |
| | | KTHTCPPCPAPELLGGPSVFL | | NRGEC | |
| | | FPPKPKDTLMISRTPEVTCVV | | | |
| | | VDVSHEDPEVKFNWYVDGVEV | | | |
| | | HNAKTKPREEQYNSTYRVVSV | | | |
| | | LTVLHQDWLNGKEYKCKVSNK | | | |
| | | ALPAPIEKTISKAKGQPREPQ | | | |
| | | VYTLPPSRDELTKNQVSLTCL | | | |
| | | VKGFYPSDIAVEWESNGQPEN | | | |
| | | NYKTTPPVLDSDGSFFLYSKL | | | |
| | | TVDKSRWQQGNVFSCSVMHEA | | | |
| | | LHNHYTQKSLSLSPGK | | | |
| CL03 | 58 | EVQLVESGGGLVQPGGSLRLS | 195 | DIQMTQSPSSLSASVGDRV | WO2022212876 |
| IqG | | CAASGFDFSSYSIHWVRQAPG | | TITCRASQSVSSAVAWYQQ | |
| H1- | | KGLEWVASIYPYSGSTYYADS | | KPGKAPKLLIYSASSLYSG | |
| NtoD | | VKGRFTISADTSKNTAYLQMN | | VPSRFSGSRSGTDFTLTIS | |
| H3- | | SLRAEDTAVYYCARQSHSYYS | | SLQPEDFATYYCQQSGQRP | |
| MtoA | | HTWWVSYGADYWGQGTLVTVS | | ITFGQGTKVEIKRTVAAPS | |
| (″IgG101″) | | SASTKGPSVFPLAPSSKSTSG | | VFIFPPSDSQLKSGTASVV | |
| | | GTAALGCLVKDYFPEPVTVSW | | CLLNNFYPREAKVQWKVDN | |
| | | NSGALTSGVHTFPAVLQSSGL | | ALQSGNSQESVTEQDSKDS | |
| | | YSLSSVVTVPSSSLGTQTYIC | | TYSLSSTLTLSKADYEKHK | |
| | | NVNHKPSNTKVDKKVEPKSCD | | VYACEVTHQGLSSPVTKSF | |
| | | KTHTCPPCPAPELLGGPSVFL | | NRGEC | |
| | | FPPKPKDTLMISRTPEVTCVV | | | |
| | | VDVSHEDPEVKFNWYVDGVEV | | | |
| | | HNAKTKPREEQYNSTYRVVSV | | | |
| | | LTVLHQDWLNGKEYKCKVSNK | | | |
| | | ALPAPIEKTISKAKGQPREPQ | | | |
| | | VYTLPPSRDELTKNQVSLTCL | | | |
| | | VKGFYPSDIAVEWESNGQPEN | | | |
| | | NYKTTPPVLDSDGSFFLYSKL | | | |
| | | TVDKSRWQQGNVFSCSVMHEA | | | |
| | | LHNHYTQKSLSLSPGK | | | |
| CL03 | 59 | EVQLVESGGGLVQPGGSLRLS | 196 | DIQMTQSPSSLSASVGDRV | WO2022212876 |
| IqG | | CAASGFDFSSYSIHWVRQAPG | | TITCRASQSVSSAVAWYQQ | |
| H1- | | KGLEWVASIYPYSGSTYYADS | | KPGKAPKLLIYSASSLYSG | |
| NtoD | | VKGRFTISADTSKNTAYLQMN | | VPSRFSGSRSGTDFTLTIS | |
| H3- | | SLRAEDTAVYYCARQSHSYYS | | SLQPEDFATYYCQQSGQRP | |
| Mtol | | HTWWVSYGIDYWGQGTLVTVS | | ITFGQGTKVEIKRTVAAPS | |
| | | SASTKGPSVFPLAPSSKSTSG | | VFIFPPSDSQLKSGTASVV | |
| | | GTAALGCLVKDYFPEPVTVSW | | CLLNNFYPREAKVQWKVDN | |
| | | NSGALTSGVHTFPAVLQSSGL | | ALQSGNSQESVTEQDSKDS | |
| | | YSLSSVVTVPSSSLGTQTYIC | | TYSLSSTLTLSKADYEKHK | |
| | | NVNHKPSNTKVDKKVEPKSCD | | VYACEVTHQGLSSPVTKSF | |
| | | KTHTCPPCPAPELLGGPSVFL | | NRGEC | |
| | | FPPKPKDTLMISRTPEVTCVV | | | |
| | | VDVSHEDPEVKFNWYVDGVEV | | | |
| | | HNAKTKPREEQYNSTYRVVSV | | | |
| | | LTVLHQDWLNGKEYKCKVSNK | | | |
| | | ALPAPIEKTISKAKGQPREPQ | | | |
| | | VYTLPPSRDELTKNQVSLTCL | | | |
| | | VKGFYPSDIAVEWESNGQPEN | | | |
| | | NYKTTPPVLDSDGSFFLYSKL | | | |
| | | TVDKSRWQQGNVFSCSVMHEA | | | |
| | | LHNHYTQKSLSLSPGK | | | |
| CL03 | 60 | EVQLVESGGGLVQPGGSLRLS | 197 | DIQMTQSPSSLSASVGDRV | WO2022212876 |
| IqG | | CAASGFDFSSYSIHWVRQAPG | | TITCRASQSVSSAVAWYQQ | |
| H1- | | KGLEWVASIYPYSGSTYYADS | | KPGKAPKLLIYSASSLYSG | |
| NtoD | | VKGRFTISADTSKNTAYLQMN | | VPSRFSGSRSGTDFTLTIS | |
| H3- | | SLRAEDTAVYYCARQSHSYYS | | SLQPEDFATYYCQQSGQRP | |
| MtoL | | HTWWVSYGLDYWGQGTLVTVS | | ITFGQGTKVEIKRTVAAPS | |
| | | SASTKGPSVFPLAPSSKSTSG | | VFIFPPSDSQLKSGTASVV | |
| | | GTAALGCLVKDYFPEPVTVSW | | CLLNNFYPREAKVQWKVDN | |
| | | NSGALTSGVHTFPAVLQSSGL | | ALQSGNSQESVTEQDSKDS | |
| | | YSLSSVVTVPSSSLGTQTYIC | | TYSLSSTLTLSKADYEKHK | |
| | | NVNHKPSNTKVDKKVEPKSCD | | VYACEVTHQGLSSPVTKSF | |
| | | KTHTCPPCPAPELLGGPSVFL | | NRGEC | |
| | | FPPKPKDTLMISRTPEVTCVV | | | |
| | | VDVSHEDPEVKFNWYVDGVEV | | | |
| | | HNAKTKPREEQYNSTYRVVSV | | | |
| | | LTVLHQDWLNGKEYKCKVSNK | | | |
| | | ALPAPIEKTISKAKGQPREPQ | | | |
| | | VYTLPPSRDELTKNQVSLTCL | | | |
| | | VKGFYPSDIAVEWESNGQPEN | | | |
| | | NYKTTPPVLDSDGSFFLYSKL | | | |
| | | TVDKSRWQQGNVFSCSVMHEA | | | |
| | | LHNHYTQKSLSLSPGK | | | |
| CL03 | 61 | EVQLVESGGGLVQPGGSLRLS | 198 | DIQMTQSPSSLSASVGDRV | WO2022212876 |
| IgG | | CAASGFDFSSYSIHWVRQAPG | | TITCRASQSVSSAVAWYQQ | |
| H1- | | KGLEWVASIYPYSGSTYYADS | | KPGKAPKLLIYSASSLYSG | |
| NtoD | | VKGRFTISADTSKNTAYLQMN | | VPSRFSGSRSGTDFTLTIS | |
| H3- | | SLRAEDTAVYYCARQSHSYYS | | SLQPEDFATYYCQQSGQRP | |
| MtoV | | HTWWVSYGVDYWGQGTLVTVS | | ITFGQGTKVEIKRTVAAPS | |
| | | SASTKGPSVFPLAPSSKSTSG | | VFIFPPSDSQLKSGTASVV | |
| | | GTAALGCLVKDYFPEPVTVSW | | CLLNNFYPREAKVQWKVDN | |
| | | NSGALTSGVHTFPAVLQSSGL | | ALQSGNSQESVTEQDSKDS | |
| | | YSLSSVVTVPSSSLGTQTYIC | | TYSLSSTLTLSKADYEKHK | |
| | | NVNHKPSNTKVDKKVEPKSCD | | VYACEVTHQGLSSPVTKSF | |
| | | KTHTCPPCPAPELLGGPSVFL | | NRGEC | |
| | | FPPKPKDTLMISRTPEVTCVV | | | |
| | | VDVSHEDPEVKFNWYVDGVEV | | | |
| | | HNAKTKPREEQYNSTYRVVSV | | | |
| | | LTVLHQDWLNGKEYKCKVSNK | | | |
| | | ALPAPIEKTISKAKGQPREPQ | | | |
| | | VYTLPPSRDELTKNQVSLTCL | | | |
| | | VKGFYPSDIAVEWESNGQPEN | | | |
| | | NYKTTPPVLDSDGSFFLYSKL | | | |
| | | TVDKSRWQQGNVFSCSVMHEA | | | |
| | | LHNHYTQKSLSLSPGK | | | |
| CL07 | 62 | EISEVQLVESGGGLVQPGGSL | 199 | MASDIQMTQSPSSLSASVG | WO2022212876 |
| Fab | | RLSCAASGFNFSSSSIHWVRQ | | DRVTITCRASQSVSSAVAW | |
| | | APGKGLEWVASIYPYSGSTSY | | YQQKPGKAPKLLIYSASSL | |
| | | ADSVKGRFTISADTSKNTAYL | | YSGVPSRFSGSRSGTDFTL | |
| | | QMNSLRAEDTAVYYCARTYWV | | TISSLQPEDFATYYCQQSS | |
| | | QFWYGHFSYYRPALDYWGQGT | | SSLITFGQGTKVEIKRTVA | |
| | | LVTVSSASTKGPSVFPLAPSS | | APSVFIFPPSDSQLKSGTA | |
| | | KSTSGGTAALGCLVKDYFPEP | | SVVCLLNNFYPREAKVQWK | |
| | | VTVSWNSGALTSGVHTFPAVL | | VDNALQSGNSQESVTEQDS | |
| | | QSSGLYSLSSVVTVPSSSLGT | | KDSTYSLSSTLTLSKADYE | |
| | | QTYICNVNHKPSNTKVDKKVE | | KHKVYACEVTHQGLSSPVT | |
| | | PKSC | | KSFNRGEC | |
| CL07 | 63 | EVQLVESGGGLVQPGGSLRLS | 200 | DIQMTQSPSSLSASVGDRV | WO2022212876 |
| IgG | | CAASGFNFSSSSIHWVRQAPG | | TITCRASQSVSSAVAWYQQ | |
| | | KGLEWVASIYPYSGSTSYADS | | KPGKAPKLLIYSASSLYSG | |
| | | VKGRFTISADTSKNTAYLQMN | | VPSRFSGSRSGTDFTLTIS | |
| | | SLRAEDTAVYYCARTYWVQFW | | SLQPEDFATYYCQQSSSSL | |
| | | YGHFSYYRPALDYWGQGTLVT | | ITFGQGTKVEIKRTVAAPS | |
| | | VSSASTKGPSVFPLAPSSKST | | VFIFPPSDSQLKSGTASVV | |
| | | SGGTAALGCLVKDYFPEPVTV | | CLLNNFYPREAKVQWKVDN | |
| | | SWNSGALTSGVHTFPAVLQSS | | ALQSGNSQESVTEQDSKDS | |
| | | GLYSLSSVVTVPSSSLGTQTY | | TYSLSSTLTLSKADYEKHK | |
| | | ICNVNHKPSNTKVDKKVEPKS | | VYACEVTHQGLSSPVTKSF | |
| | | CDKTHTCPPCPAPELLGGPSV | | NRGEC | |
| | | FLFPPKPKDTLMISRTPEVTC | | | |
| | | VVVDVSHEDPEVKFNWYVDGV | | | |
| | | EVHNAKTKPREEQYNSTYRVV | | | |
| | | SVLTVLHQDWLNGKEYKCKVS | | | |
| | | NKALPAPIEKTISKAKGQPRE | | | |
| | | PQVYTLPPSRDELTKNQVSLT | | | |
| | | CLVKGFYPSDIAVEWESNGQP | | | |
| | | ENNYKTTPPVLDSDGSFFLYS | | | |
| | | KLTVDKSRWQQGNVFSCSVMH | | | |
| | | EALHNHYTQKSLSLSPGK | | | |
| CL07 | 64 | EVQLVESGGGLVQPGGSLRLS | 201 | DIQMTQSPSSLSASVGDRV | WO2022212876 |
| IqG | | CAASGFDFSSSSIHWVRQAPG | | TITCRASQSVSSAVAWYQQ | |
| H1- | | KGLEWVASIYPYSGSTSYADS | | KPGKAPKLLIYSASSLYSG | |
| NtoD | | VKGRFTISADTSKNTAYLQMN | | VPSRFSGSRSGTDFTLTIS | |
| | | SLRAEDTAVYYCARTYWVQFW | | SLQPEDFATYYCQQSSSSL | |
| | | YGHFSYYRPALDYWGQGTLVT | | ITFGQGTKVEIKRTVAAPS | |
| | | VSSASTKGPSVFPLAPSSKST | | VFIFPPSDSQLKSGTASVV | |
| | | SGGTAALGCLVKDYFPEPVTV | | CLLNNFYPREAKVQWKVDN | |
| | | SWNSGALTSGVHTFPAVLQSS | | ALQSGNSQESVTEQDSKDS | |
| | | GLYSLSSVVTVPSSSLGTQTY | | TYSLSSTLTLSKADYEKHK | |
| | | ICNVNHKPSNTKVDKKVEPKS | | VYACEVTHQGLSSPVTKSF | |
| | | CDKTHTCPPCPAPELLGGPSV | | NRGEC | |
| | | FLFPPKPKDTLMISRTPEVTC | | | |
| | | VVVDVSHEDPEVKFNWYVDGV | | | |
| | | EVHNAKTKPREEQYNSTYRVV | | | |
| | | SVLTVLHQDWLNGKEYKCKVS | | | |
| | | NKALPAPIEKTISKAKGQPRE | | | |
| | | PQVYTLPPSRDELTKNQVSLT | | | |
| | | CLVKGFYPSDIAVEWESNGQP | | | |
| | | ENNYKTTPPVLDSDGSFFLYS | | | |
| | | KLTVDKSRWQQGNVFSCSVMH | | | |
| | | EALHNHYTQKSLSLSPGK | | | |
| IgG3H7T | 65 | EVQLVESGGGLVQPGGSLRLS | 202 | MTQSPSSLSASVGDRVTIT | WO2022212876 |
| | | CAASGFDFSSYSIHWVRQAPG | | CRASQSVSSAVAWYQQKPG | |
| | | KGLEWVASIYPYSGSTYYADS | | KAPKLLIYSASSLYSGVPS | |
| | | VKGRFTISADTSKNTAYLQMN | | RFSGSRSGTDFTLTISSLQ | |
| | | SLRAEDTAVYYCARQSHSYYS | | PEDFATYYCQQSSSSLITF | |
| | | HTWWVSYGMDYWGQGTLVTVS | | GQGTKVEIKRTVAAPSVFI | |
| | | SASTKGPSVFPLAPSSKSTSG | | FPPSDSQLKSGTASVVCLL | |
| | | GTAALGCLVKDYFPEPVTVSW | | NNFYPREAKVQWKVDNALQ | |
| | | NSGALTSGVHTFPAVLQSSGL | | SGNSQESVTEQDSKDSTYS | |
| | | YSLSSVVTVPSSSLGTQTYIC | | LSSTLTLSKADYEKHKVYA | |
| | | NVNHKPSNTKVDKKVEPKSCD | | CEVTHQGLSSPVTKSFNRG | |
| | | KTHTCPPCPAPELLGGPSVFL | | EC | |
| | | FPPKPKDTLMISRTPEVTCVV | | | |
| | | VDVSHEDPEVKFNWYVDGVEV | | | |
| | | HNAKTKPREEQYNSTYRVVSV | | | |
| | | LTVLHQDWLNGKEYKCKVSNK | | | |
| | | ALPAPIEKTISKAKGQPREPQ | | | |
| | | VYTLPPSRDELTKNQVSLTCL | | | |
| | | VKGFYPSDIAVEWESNGQPEN | | | |
| | | NYKTTPPVLDSDGSFFLYSKL | | | |
| | | TVDKSRWQQGNVFSCSVMHEA | | | |
| | | LHNHYTQKSLSLSPGK | | | |
| IgGA01 | 66 | EVQLVESGGGLVQPGGSLRLS | 203 | MTQSPSSLSASVGDRVTIT | WO2022212876 |
| | | CAASGFDFSSYSIHWVRQAPG | | CRASQSVSSAVAWYQQKPG | |
| | | KGLEWVASIYPYSGSTYYADS | | KAPKLLIYSASSLYSGVPS | |
| | | VKGRFTISADTSKNTAYLQMN | | RFSGSRSGTDFTLTISSLQ | |
| | | SLRAEDTAVYYCARQNHSYYS | | PEDFATYYCQQSGQRPITF | |
| | | HTWWVSYGMDYWGQGTLVTVS | | GQGTKVEIKRTVAAPSVFI | |
| | | SASTKGPSVFPLAPSSKSTSG | | FPPSDSQLKSGTASVVCLL | |
| | | GTAALGCLVKDYFPEPVTVSW | | NNFYPREAKVQWKVDNALQ | |
| | | NSGALTSGVHTFPAVLQSSGL | | SGNSQESVTEQDSKDSTYS | |
| | | YSLSSVVTVPSSSLGTQTYIC | | LSSTLTLSKADYEKHKVYA | |
| | | NVNHKPSNTKVDKKVEPKSCD | | CEVTHQGLSSPVTKSFNRG | |
| | | KTHTCPPCPAPELLGGPSVFL | | EC | |
| | | FPPKPKDTLMISRTPEVTCVV | | | |
| | | VDVSHEDPEVKFNWYVDGVEV | | | |
| | | HNAKTKPREEQYNSTYRVVSV | | | |
| | | LTVLHQDWLNGKEYKCKVSNK | | | |
| | | ALPAPIEKTISKAKGQPREPQ | | | |
| | | VYTLPPSRDELTKNQVSLTCL | | | |
| | | VKGFYPSDIAVEWESNGQPEN | | | |
| | | NYKTTPPVLDSDGSFFLYSKL | | | |
| | | TVDKSRWQQGNVFSCSVMHEA | | | |
| | | LHNHYTQKSLSLSPGK | | | |
| IgGA03 | 67 | EVQLVESGGGLVQPGGSLRLS | 204 | MTQSPSSLSASVGDRVTIT | WO2022212876 |
| | | CAASGFDFSSYSIHWVRQAPG | | CRASQSVSSAVAWYQQKPG | |
| | | KGLEWVASIYPYSGSTYYADS | | KAPKLLIYSASSLYSGVPS | |
| | | VKGRFTISADTSKNTAYLQMN | | RFSGSRSGTDFTLTISSLQ | |
| | | SLRAEDTAVYYCARQTHSYYS | | PEDFATYYCQQSGQRPITF | |
| | | HTWWVSYGMDYWGQGTLVTVS | | GQGTKVEIKRTVAAPSVFI | |
| | | SASTKGPSVFPLAPSSKSTSG | | FPPSDSQLKSGTASVVCLL | |
| | | GTAALGCLVKDYFPEPVTVSW | | NNFYPREAKVQWKVDNALQ | |
| | | NSGALTSGVHTFPAVLQSSGL | | SGNSQESVTEQDSKDSTYS | |
| | | YSLSSVVTVPSSSLGTQTYIC | | LSSTLTLSKADYEKHKVYA | |
| | | NVNHKPSNTKVDKKVEPKSCD | | CEVTHQGLSSPVTKSFNRG | |
| | | KTHTCPPCPAPELLGGPSVFL | | EC | |
| | | FPPKPKDTLMISRTPEVTCVV | | | |
| | | VDVSHEDPEVKFNWYVDGVEV | | | |
| | | HNAKTKPREEQYNSTYRVVSV | | | |
| | | LTVLHQDWLNGKEYKCKVSNK | | | |
| | | ALPAPIEKTISKAKGQPREPQ | | | |
| | | VYTLPPSRDELTKNQVSLTCL | | | |
| | | VKGFYPSDIAVEWESNGQPEN | | | |
| | | NYKTTPPVLDSDGSFFLYSKL | | | |
| | | TVDKSRWQQGNVFSCSVMHEA | | | |
| | | LHNHYTQKSLSLSPGK | | | |
| IgGA04 | 68 | EVQLVESGGGLVQPGGSLRLS | 205 | MTQSPSSLSASVGDRVTIT | WO2022212876 |
| | | CAASGFDFSSYSIHWVRQAPG | | CRASQSVSSAVAWYQQKPG | |
| | | KGLEWVASIYPYSGSTYYADS | | KAPKLLIYSASSLYSGVPS | |
| | | VKGRFTISADTSKNTAYLQMN | | RFSGSRSGTDFTLTISSLQ | |
| | | SLRAEDTAVYYCARQGHSYYS | | PEDFATYYCQQSGQRPITF | |
| | | HTWWVSYGMDYWGQGTLVTVS | | GQGTKVEIKRTVAAPSVFI | |
| | | SASTKGPSVFPLAPSSKSTSG | | FPPSDSQLKSGTASVVCLL | |
| | | GTAALGCLVKDYFPEPVTVSW | | NNFYPREAKVQWKVDNALQ | |
| | | NSGALTSGVHTFPAVLQSSGL | | SGNSQESVTEQDSKDSTYS | |
| | | YSLSSVVTVPSSSLGTQTYIC | | LSSTLTLSKADYEKHKVYA | |
| | | NVNHKPSNTKVDKKVEPKSCD | | CEVTHQGLSSPVTKSFNRG | |
| | | KTHTCPPCPAPELLGGPSVFL | | EC | |
| | | FPPKPKDTLMISRTPEVTCVV | | | |
| | | VDVSHEDPEVKFNWYVDGVEV | | | |
| | | HNAKTKPREEQYNSTYRVVSV | | | |
| | | LTVLHQDWLNGKEYKCKVSNK | | | |
| | | ALPAPIEKTISKAKGQPREPQ | | | |
| | | VYTLPPSRDELTKNQVSLTCL | | | |
| | | VKGFYPSDIAVEWESNGQPEN | | | |
| | | NYKTTPPVLDSDGSFFLYSKL | | | |
| | | TVDKSRWQQGNVFSCSVMHEA | | | |
| | | LHNHYTQKSLSLSPGK | | | |
| IgGA05 | 69 | EVQLVESGGGLVQPGGSLRLS | 206 | MTQSPSSLSASVGDRVTIT | WO2022212876 |
| | | CAASGFDFSSYSIHWVRQAPG | | CRASQSVSSAVAWYQQKPG | |
| | | KGLEWVASIYPYSGSTYYADS | | KAPKLLIYSASSLYSGVPS | |
| | | VKGRFTISADTSKNTAYLQMN | | RFSGSRSGTDFTLTISSLQ | |
| | | SLRAEDTAVYYCARQAHSYYS | | PEDFATYYCQQSGQRPITF | |
| | | HTWWVSYGMDYWGQGTLVTVS | | GQGTKVEIKRTVAAPSVFI | |
| | | SASTKGPSVFPLAPSSKSTSG | | FPPSDSQLKSGTASVVCLL | |
| | | GTAALGCLVKDYFPEPVTVSW | | NNFYPREAKVQWKVDNALQ | |
| | | NSGALTSGVHTFPAVLQSSGL | | SGNSQESVTEQDSKDSTYS | |
| | | YSLSSVVTVPSSSLGTQTYIC | | LSSTLTLSKADYEKHKVYA | |
| | | NVNHKPSNTKVDKKVEPKSCD | | CEVTHQGLSSPVTKSFNRG | |
| | | KTHTCPPCPAPELLGGPSVFL | | EC | |
| | | FPPKPKDTLMISRTPEVTCVV | | | |
| | | VDVSHEDPEVKFNWYVDGVEV | | | |
| | | HNAKTKPREEQYNSTYRVVSV | | | |
| | | LTVLHQDWLNGKEYKCKVSNK | | | |
| | | ALPAPIEKTISKAKGQPREPQ | | | |
| | | VYTLPPSRDELTKNQVSLTCL | | | |
| | | VKGFYPSDIAVEWESNGQPEN | | | |
| | | NYKTTPPVLDSDGSFFLYSKL | | | |
| | | TVDKSRWQQGNVFSCSVMHEA | | | |
| | | LHNHYTQKSLSLSPGK | | | |
| IgGA06 | 70 | EVQLVESGGGLVQPGGSLRLS | 207 | MTQSPSSLSASVGDRVTIT | WO2022212876 |
| | | CAASGFDFSSYSIHWVRQAPG | | CRASQSVSSAVAWYQQKPG | |
| | | KGLEWVASIYPYSGSTYYADS | | KAPKLLIYSASSLYSGVPS | |
| | | VKGRFTISADTSKNTAYLQMN | | RFSGSRSGTDFTLTISSLQ | |
| | | SLRAEDTAVYYCARQGQSYYS | | PEDFATYYCQQSGQRPITF | |
| | | HTWWVSYGMDYWGQGTLVTVS | | GQGTKVEIKRTVAAPSVFI | |
| | | SASTKGPSVFPLAPSSKSTSG | | FPPSDSQLKSGTASVVCLL | |
| | | GTAALGCLVKDYFPEPVTVSW | | NNFYPREAKVQWKVDNALQ | |
| | | NSGALTSGVHTFPAVLQSSGL | | SGNSQESVTEQDSKDSTYS | |
| | | YSLSSVVTVPSSSLGTQTYIC | | LSSTLTLSKADYEKHKVYA | |
| | | NVNHKPSNTKVDKKVEPKSCD | | CEVTHQGLSSPVTKSENRG | |
| | | KTHTCPPCPAPELLGGPSVFL | | EC | |
| | | FPPKPKDTLMISRTPEVTCVV | | | |
| | | VDVSHEDPEVKFNWYVDGVEV | | | |
| | | HNAKTKPREEQYNSTYRVVSV | | | |
| | | LTVLHQDWLNGKEYKCKVSNK | | | |
| | | ALPAPIEKTISKAKGQPREPQ | | | |
| | | VYTLPPSRDELTKNQVSLTCL | | | |
| | | VKGFYPSDIAVEWESNGQPEN | | | |
| | | NYKTTPPVLDSDGSFFLYSKL | | | |
| | | TVDKSRWQQGNVFSCSVMHEA | | | |
| | | LHNHYTQKSLSLSPGK | | | |
| IgGA08 | 71 | EVQLVESGGGLVQPGGSLRLS | 208 | MTQSPSSLSASVGDRVTIT | WO2022212876 |
| | | CAASGFDFSSYSIHWVRQAPG | | CRASQSVSSAVAWYQQKPG | |
| | | KGLEWVASIYPYSGSTYYADS | | KAPKLLIYSASSLYSGVPS | |
| | | VKGRFTISADTSKNTAYLQMN | | RFSGSRSGTDFTLTISSLQ | |
| | | SLRAEDTAVYYCARQSHSYYS | | PEDFATYYCQQSGERPITF | |
| | | HTWWVSYGMDYWGQGTLVTVS | | GQGTKVEIKRTVAAPSVFI | |
| | | SASTKGPSVFPLAPSSKSTSG | | FPPSDSQLKSGTASVVCLL | |
| | | GTAALGCLVKDYFPEPVTVSW | | NNFYPREAKVQWKVDNALQ | |
| | | NSGALTSGVHTFPAVLQSSGL | | SGNSQESVTEQDSKDSTYS | |
| | | YSLSSVVTVPSSSLGTQTYIC | | LSSTLTLSKADYEKHKVYA | |
| | | NVNHKPSNTKVDKKVEPKSCD | | CEVTHQGLSSPVTKSFNRG | |
| | | KTHTCPPCPAPELLGGPSVFL | | EC | |
| | | FPPKPKDTLMISRTPEVTCVV | | | |
| | | VDVSHEDPEVKFNWYVDGVEV | | | |
| | | HNAKTKPREEQYNSTYRVVSV | | | |
| | | LTVLHQDWLNGKEYKCKVSNK | | | |
| | | ALPAPIEKTISKAKGQPREPQ | | | |
| | | VYTLPPSRDELTKNQVSLTCL | | | |
| | | VKGFYPSDIAVEWESNGQPEN | | | |
| | | NYKTTPPVLDSDGSFFLYSKL | | | |
| | | TVDKSRWQQGNVFSCSVMHEA | | | |
| | | LHNHYTQKSLSLSPGK | | | |
| IgGA09 | 72 | EVQLVESGGGLVQPGGSLRLS | 209 | MTQSPSSLSASVGDRVTIT | WO2022212876 |
| | | CAASGFDFSSYSIHWVRQAPG | | CRASQSVSSAVAWYQQKPG | |
| | | KGLEWVASIYPYSGSTYYADS | | KAPKLLIYSASSLYSGVPS | |
| | | VKGRFTISADTSKNTAYLQMN | | RFSGSRSGTDFTLTISSLQ | |
| | | SLRAEDTAVYYCARQSHSYYS | | PEDFATYYCQQSGNVPATF | |
| | | HTWWVSYGMDYWGQGTLVTVS | | GQGTKVEIKRTVAAPSVFI | |
| | | SASTKGPSVFPLAPSSKSTSG | | FPPSDSQLKSGTASVVCLL | |
| | | GTAALGCLVKDYFPEPVTVSW | | NNFYPREAKVQWKVDNALQ | |
| | | NSGALTSGVHTFPAVLQSSGL | | SGNSQESVTEQDSKDSTYS | |
| | | YSLSSVVTVPSSSLGTQTYIC | | LSSTLTLSKADYEKHKVYA | |
| | | NVNHKPSNTKVDKKVEPKSCD | | CEVTHQGLSSPVTKSENRG | |
| | | KTHTCPPCPAPELLGGPSVFL | | EC | |
| | | FPPKPKDTLMISRTPEVTCVV | | | |
| | | VDVSHEDPEVKFNWYVDGVEV | | | |
| | | HNAKTKPREEQYNSTYRVVSV | | | |
| | | LTVLHQDWLNGKEYKCKVSNK | | | |
| | | ALPAPIEKTISKAKGQPREPQ | | | |
| | | VYTLPPSRDELTKNQVSLTCL | | | |
| | | VKGFYPSDIAVEWESNGQPEN | | | |
| | | NYKTTPPVLDSDGSFFLYSKL | | | |
| | | TVDKSRWQQGNVFSCSVMHEA | | | |
| | | LHNHYTQKSLSLSPGK | | | |
| IgGA10 | 73 | EVQLVESGGGLVQPGGSLRLS | 210 | MTQSPSSLSASVGDRVTIT | WO2022212876 |
| | | CAASGFDFSSYSIHWVRQAPG | | CRASQSVSSAVAWYQQKPG | |
| | | KGLEWVASIYPYSGSTYYADS | | KAPKLLIYSASSLYSGVPS | |
| | | VKGRFTISADTSKNTAYLQMN | | RFSGSRSGTDFTLTISSLQ | |
| | | SLRAEDTAVYYCARQSHSYYS | | PEDFATYYCQQSGKRPMTF | |
| | | HTWWVSYGMDYWGQGTLVTVS | | GQGTKVEIKRTVAAPSVFI | |
| | | SASTKGPSVFPLAPSSKSTSG | | FPPSDSQLKSGTASVVCLL | |
| | | GTAALGCLVKDYFPEPVTVSW | | NNFYPREAKVQWKVDNALQ | |
| | | NSGALTSGVHTFPAVLQSSGL | | SGNSQESVTEQDSKDSTYS | |
| | | YSLSSVVTVPSSSLGTQTYIC | | LSSTLTLSKADYEKHKVYA | |
| | | NVNHKPSNTKVDKKVEPKSCD | | CEVTHQGLSSPVTKSENRG | |
| | | KTHTCPPCPAPELLGGPSVFL | | EC | |
| | | FPPKPKDTLMISRTPEVTCVV | | | |
| | | VDVSHEDPEVKFNWYVDGVEV | | | |
| | | HNAKTKPREEQYNSTYRVVSV | | | |
| | | LTVLHQDWLNGKEYKCKVSNK | | | |
| | | ALPAPIEKTISKAKGQPREPQ | | | |
| | | VYTLPPSRDELTKNQVSLTCL | | | |
| | | VKGFYPSDIAVEWESNGQPEN | | | |
| | | NYKTTPPVLDSDGSFFLYSKL | | | |
| | | TVDKSRWQQGNVFSCSVMHEA | | | |
| | | LHNHYTQKSLSLSPGK | | | |
| IgGA11 | 74 | EVQLVESGGGLVQPGGSLRLS | 211 | MTQSPSSLSASVGDRVTIT | WO2022212876 |
| | | CAASGFDFSSYSIHWVRQAPG | | CRASQSVSSAVAWYQQKPG | |
| | | KGLEWVASIYPYSGSTYYADS | | KAPKLLIYSASSLYSGVPS | |
| | | VKGRFTISADTSKNTAYLQMN | | RFSGSRSGTDFTLTISSLQ | |
| | | SLRAEDTAVYYCARRSHSYYS | | PEDFATYYCQQTGQRPMTF | |
| | | HTWWVSYGMDYWGQGTLVTVS | | GQGTKVEIKRTVAAPSVFI | |
| | | SASTKGPSVFPLAPSSKSTSG | | FPPSDSQLKSGTASVVCLL | |
| | | GTAALGCLVKDYFPEPVTVSW | | NNFYPREAKVQWKVDNALQ | |
| | | NSGALTSGVHTFPAVLQSSGL | | SGNSQESVTEQDSKDSTYS | |
| | | YSLSSVVTVPSSSLGTQTYIC | | LSSTLTLSKADYEKHKVYA | |
| | | NVNHKPSNTKVDKKVEPKSCD | | CEVTHQGLSSPVTKSFNRG | |
| | | KTHTCPPCPAPELLGGPSVFL | | EC | |
| | | FPPKPKDTLMISRTPEVTCVV | | | |
| | | VDVSHEDPEVKFNWYVDGVEV | | | |
| | | HNAKTKPREEQYNSTYRVVSV | | | |
| | | LTVLHQDWLNGKEYKCKVSNK | | | |
| | | ALPAPIEKTISKAKGQPREPQ | | | |
| | | VYTLPPSRDELTKNQVSLTCL | | | |
| | | VKGFYPSDIAVEWESNGQPEN | | | |
| | | NYKTTPPVLDSDGSFFLYSKL | | | |
| | | TVDKSRWQQGNVFSCSVMHEA | | | |
| | | LHNHYTQKSLSLSPGK | | | |
| IgGB01 | 75 | EVQLVESGGGLVQPGGSLRLS | 212 | MTQSPSSLSASVGDRVTIT | WO2022212876 |
| | | CAASGFDFSSYSIHWVRQAPG | | CRASQSVSSAVAWYQQKPG | |
| | | KGLEWVASIYPYSGSTYYADS | | KAPKLLIYSASSLYSGVPS | |
| | | VKGRFTISADTSKNTAYLQMN | | RFSGSRSGTDFTLTISSLQ | |
| | | SLRAEDTAVYYCARQSHSYYS | | PEDFATYYCQQSMQWPKTF | |
| | | HTWWVSYGMDYWGQGTLVTVS | | GQGTKVEIKRTVAAPSVFI | |
| | | SASTKGPSVFPLAPSSKSTSG | | FPPSDSQLKSGTASVVCLL | |
| | | GTAALGCLVKDYFPEPVTVSW | | NNFYPREAKVQWKVDNALQ | |
| | | NSGALTSGVHTFPAVLQSSGL | | SGNSQESVTEQDSKDSTYS | |
| | | YSLSSVVTVPSSSLGTQTYIC | | LSSTLTLSKADYEKHKVYA | |
| | | NVNHKPSNTKVDKKVEPKSCD | | CEVTHQGLSSPVTKSFNRG | |
| | | KTHTCPPCPAPELLGGPSVFL | | EC | |
| | | FPPKPKDTLMISRTPEVTCVV | | | |
| | | VDVSHEDPEVKFNWYVDGVEV | | | |
| | | HNAKTKPREEQYNSTYRVVSV | | | |
| | | LTVLHQDWLNGKEYKCKVSNK | | | |
| | | ALPAPIEKTISKAKGQPREPQ | | | |
| | | VYTLPPSRDELTKNQVSLTCL | | | |
| | | VKGFYPSDIAVEWESNGQPEN | | | |
| | | NYKTTPPVLDSDGSFFLYSKL | | | |
| | | TVDKSRWQQGNVFSCSVMHEA | | | |
| | | LHNHYTQKSLSLSPGK | | | |
| IgGB03 | 76 | EVQLVESGGGLVQPGGSLRLS | 213 | MTQSPSSLSASVGDRVTIT | WO2022212876 |
| | | CAASGFDFSSYSIHWVRQAPG | | CRASQSVSSAVAWYQQKPG | |
| | | KGLEWVASIYPYSGSTYYADS | | KAPKLLIYSASSLYSGVPS | |
| | | VKGRFTISADTSKNTAYLQMN | | RFSGSRSGTDFTLTISSLQ | |
| | | SLRAEDTAVYYCARQSHSFYS | | PEDFATYYCQQSLQRPMTF | |
| | | HTWWVSYGMDYWGQGTLVTVS | | GQGTKVEIKRTVAAPSVFI | |
| | | SASTKGPSVFPLAPSSKSTSG | | FPPSDSQLKSGTASVVCLL | |
| | | GTAALGCLVKDYFPEPVTVSW | | NNFYPREAKVQWKVDNALQ | |
| | | NSGALTSGVHTFPAVLQSSGL | | SGNSQESVTEQDSKDSTYS | |
| | | YSLSSVVTVPSSSLGTQTYIC | | LSSTLTLSKADYEKHKVYA | |
| | | NVNHKPSNTKVDKKVEPKSCD | | CEVTHQGLSSPVTKSFNRG | |
| | | KTHTCPPCPAPELLGGPSVFL | | EC | |
| | | FPPKPKDTLMISRTPEVTCVV | | | |
| | | VDVSHEDPEVKFNWYVDGVEV | | | |
| | | HNAKTKPREEQYNSTYRVVSV | | | |
| | | LTVLHQDWLNGKEYKCKVSNK | | | |
| | | ALPAPIEKTISKAKGQPREPQ | | | |
| | | VYTLPPSRDELTKNQVSLTCL | | | |
| | | VKGFYPSDIAVEWESNGQPEN | | | |
| | | NYKTTPPVLDSDGSFFLYSKL | | | |
| | | TVDKSRWQQGNVFSCSVMHEA | | | |
| | | LHNHYTQKSLSLSPGK | | | |
| IgGB04 | 77 | EVQLVESGGGLVQPGGSLRLS | 214 | MTQSPSSLSASVGDRVTIT | WO2022212876 |
| | | CAASGFDFSSYSIHWVRQAPG | | CRASQSVSSAVAWYQQKPG | |
| | | KGLEWVASIYPYSGSTYYADS | | KAPKLLIYSASSLYSGVPS | |
| | | VKGRFTISADTSKNTAYLQMN | | RFSGSRSGTDFTLTISSLQ | |
| | | SLRAEDTAVYYCARRSHSYYS | | PEDFATYYCQQVELVPMTF | |
| | | HTWWVSYGMDYWGQGTLVTVS | | GQGTKVEIKRTVAAPSVFI | |
| | | SASTKGPSVFPLAPSSKSTSG | | FPPSDSQLKSGTASVVCLL | |
| | | GTAALGCLVKDYFPEPVTVSW | | NNFYPREAKVQWKVDNALQ | |
| | | NSGALTSGVHTFPAVLQSSGL | | SGNSQESVTEQDSKDSTYS | |
| | | YSLSSVVTVPSSSLGTQTYIC | | LSSTLTLSKADYEKHKVYA | |
| | | NVNHKPSNTKVDKKVEPKSCD | | CEVTHQGLSSPVTKSFNRG | |
| | | KTHTCPPCPAPELLGGPSVFL | | EC | |
| | | FPPKPKDTLMISRTPEVTCVV | | | |
| | | VDVSHEDPEVKFNWYVDGVEV | | | |
| | | HNAKTKPREEQYNSTYRVVSV | | | |
| | | LTVLHQDWLNGKEYKCKVSNK | | | |
| | | ALPAPIEKTISKAKGQPREPQ | | | |
| | | VYTLPPSRDELTKNQVSLTCL | | | |
| | | VKGFYPSDIAVEWESNGQPEN | | | |
| | | NYKTTPPVLDSDGSFFLYSKL | | | |
| | | TVDKSRWQQGNVFSCSVMHEA | | | |
| | | LHNHYTQKSLSLSPGK | | | |
| IgGB07 | 78 | EVQLVESGGGLVQPGGSLRLS | 215 | MTQSPSSLSASVGDRVTIT | WO2022212876 |
| | | CAASGFDFSSYSIHWVRQAPG | | CRASQSVSSAVAWYQQKPG | |
| | | KGLEWVASIYPYSGSTYYADS | | KAPKLLIYSASSLYSGVPS | |
| | | VKGRFTISADTSKNTAYLQMN | | RFSGSRSGTDFTLTISSLQ | |
| | | SLRAEDTAVYYCARQDHSYYS | | PEDFATYYCQQAGKRPLTF | |
| | | HTWWVSYGMDYWGQGTLVTVS | | GQGTKVEIKRTVAAPSVFI | |
| | | SASTKGPSVFPLAPSSKSTSG | | FPPSDSQLKSGTASVVCLL | |
| | | GTAALGCLVKDYFPEPVTVSW | | NNFYPREAKVQWKVDNALQ | |
| | | NSGALTSGVHTFPAVLQSSGL | | SGNSQESVTEQDSKDSTYS | |
| | | YSLSSVVTVPSSSLGTQTYIC | | LSSTLTLSKADYEKHKVYA | |
| | | NVNHKPSNTKVDKKVEPKSCD | | CEVTHQGLSSPVTKSENRG | |
| | | KTHTCPPCPAPELLGGPSVFL | | EC | |
| | | FPPKPKDTLMISRTPEVTCVV | | | |
| | | VDVSHEDPEVKFNWYVDGVEV | | | |
| | | HNAKTKPREEQYNSTYRVVSV | | | |
| | | LTVLHQDWLNGKEYKCKVSNK | | | |
| | | ALPAPIEKTISKAKGQPREPQ | | | |
| | | VYTLPPSRDELTKNQVSLTCL | | | |
| | | VKGFYPSDIAVEWESNGQPEN | | | |
| | | NYKTTPPVLDSDGSFFLYSKL | | | |
| | | TVDKSRWQQGNVFSCSVMHEA | | | |
| | | LHNHYTQKSLSLSPGK | | | |
| IgGB08 | 79 | EVQLVESGGGLVQPGGSLRLS | 216 | MTQSPSSLSASVGDRVTIT | WO2022212876 |
| | | CAASGFDFSSYSIHWVRQAPG | | CRASQSVSSAVAWYQQKPG | |
| | | KGLEWVASIYPYSGSTYYADS | | KAPKLLIYSASSLYSGVPS | |
| | | VKGRFTISADTSKNTAYLQMN | | RFSGSRSGTDFTLTISSLQ | |
| | | SLRAEDTAVYYCARRSHSYYS | | PEDFATYYCQQSVDLTATF | |
| | | HTWWVSYGMDYWGQGTLVTVS | | GQGTKVEIKRTVAAPSVFI | |
| | | SASTKGPSVFPLAPSSKSTSG | | FPPSDSQLKSGTASVVCLL | |
| | | GTAALGCLVKDYFPEPVTVSW | | NNFYPREAKVQWKVDNALQ | |
| | | NSGALTSGVHTFPAVLQSSGL | | SGNSQESVTEQDSKDSTYS | |
| | | YSLSSVVTVPSSSLGTQTYIC | | LSSTLTLSKADYEKHKVYA | |
| | | NVNHKPSNTKVDKKVEPKSCD | | CEVTHQGLSSPVTKSFNRG | |
| | | KTHTCPPCPAPELLGGPSVFL | | EC | |
| | | FPPKPKDTLMISRTPEVTCVV | | | |
| | | VDVSHEDPEVKFNWYVDGVEV | | | |
| | | HNAKTKPREEQYNSTYRVVSV | | | |
| | | LTVLHQDWLNGKEYKCKVSNK | | | |
| | | ALPAPIEKTISKAKGQPREPQ | | | |
| | | VYTLPPSRDELTKNQVSLTCL | | | |
| | | VKGFYPSDIAVEWESNGQPEN | | | |
| | | NYKTTPPVLDSDGSFFLYSKL | | | |
| | | TVDKSRWQQGNVFSCSVMHEA | | | |
| | | LHNHYTQKSLSLSPGK | | | |
| IgGC01 | 80 | EVQLVESGGGLVQPGGSLRLS | 217 | MTQSPSSLSASVGDRVTIT | WO2022212876 |
| | | CAASGFDFSSYSIHWVRQAPG | | CRASQSVSSAVAWYQQKPG | |
| | | KGLEWVASIYPYSGSTYYADS | | KAPKLLIYSASSLYSGVPS | |
| | | VKGRFTISADTSKNTAYLQMN | | RFSGSRSGTDFTLTISSLQ | |
| | | SLRAEDTAVYYCARRSHSYYS | | PEDFATYYCQQLGVRAATF | |
| | | HTWWVSYGMDYWGQGTLVTVS | | GQGTKVEIKRTVAAPSVFI | |
| | | SASTKGPSVFPLAPSSKSTSG | | FPPSDSQLKSGTASVVCLL | |
| | | GTAALGCLVKDYFPEPVTVSW | | NNFYPREAKVQWKVDNALQ | |
| | | NSGALTSGVHTFPAVLQSSGL | | SGNSQESVTEQDSKDSTYS | |
| | | YSLSSVVTVPSSSLGTQTYIC | | LSSTLTLSKADYEKHKVYA | |
| | | NVNHKPSNTKVDKKVEPKSCD | | CEVTHQGLSSPVTKSFNRG | |
| | | KTHTCPPCPAPELLGGPSVFL | | EC | |
| | | FPPKPKDTLMISRTPEVTCVV | | | |
| | | VDVSHEDPEVKFNWYVDGVEV | | | |
| | | HNAKTKPREEQYNSTYRVVSV | | | |
| | | LTVLHQDWLNGKEYKCKVSNK | | | |
| | | ALPAPIEKTISKAKGQPREPQ | | | |
| | | VYTLPPSRDELTKNQVSLTCL | | | |
| | | VKGFYPSDIAVEWESNGQPEN | | | |
| | | NYKTTPPVLDSDGSFFLYSKL | | | |
| | | TVDKSRWQQGNVFSCSVMHEA | | | |
| | | LHNHYTQKSLSLSPGK | | | |
| IgGC03 | 81 | EVQLVESGGGLVQPGGSLRLS | 218 | MTQSPSSLSASVGDRVTIT | WO2022212876 |
| | | CAASGFDFSSYSIHWVRQAPG | | CRASQSVSSAVAWYQQKPG | |
| | | KGLEWVASIYPYSGSTYYADS | | KAPKLLIYSASSLYSGVPS | |
| | | VKGRFTISADTSKNTAYLQMN | | RFSGSRSGTDFTLTISSLQ | |
| | | SLRAEDTAVYYCARQSHSYYS | | PEDFATYYCQQSGRKPVTF | |
| | | HTWWVSYGMDYWGQGTLVTVS | | GQGTKVEIKRTVAAPSVFI | |
| | | SASTKGPSVFPLAPSSKSTSG | | FPPSDSQLKSGTASVVCLL | |
| | | GTAALGCLVKDYFPEPVTVSW | | NNFYPREAKVQWKVDNALQ | |
| | | NSGALTSGVHTFPAVLQSSGL | | SGNSQESVTEQDSKDSTYS | |
| | | YSLSSVVTVPSSSLGTQTYIC | | LSSTLTLSKADYEKHKVYA | |
| | | NVNHKPSNTKVDKKVEPKSCD | | CEVTHQGLSSPVTKSFNRG | |
| | | KTHTCPPCPAPELLGGPSVFL | | EC | |
| | | FPPKPKDTLMISRTPEVTCVV | | | |
| | | VDVSHEDPEVKFNWYVDGVEV | | | |
| | | HNAKTKPREEQYNSTYRVVSV | | | |
| | | LTVLHQDWLNGKEYKCKVSNK | | | |
| | | ALPAPIEKTISKAKGQPREPQ | | | |
| | | VYTLPPSRDELTKNQVSLTCL | | | |
| | | VKGFYPSDIAVEWESNGQPEN | | | |
| | | NYKTTPPVLDSDGSFFLYSKL | | | |
| | | TVDKSRWQQGNVFSCSVMHEA | | | |
| | | LHNHYTQKSLSLSPGK | | | |
| IgGC04 | 82 | EVQLVESGGGLVQPGGSLRLS | 219 | MTQSPSSLSASVGDRVTIT | WO2022212876 |
| | | CAASGFDFSSYSIHWVRQAPG | | CRASQSVSSAVAWYQQKPG | |
| | | KGLEWVASIYPYSGSTYYADS | | KAPKLLIYSASSLYSGVPS | |
| | | VKGRFTISADTSKNTAYLQMN | | RFSGSRSGTDFTLTISSLQ | |
| | | SLRAEDTAVYYCARRSHSYYS | | PEDFATYYCQQTAQSPLTF | |
| | | HTWWVSYGMDYWGQGTLVTVS | | GQGTKVEIKRTVAAPSVFI | |
| | | SASTKGPSVFPLAPSSKSTSG | | FPPSDSQLKSGTASVVCLL | |
| | | GTAALGCLVKDYFPEPVTVSW | | NNFYPREAKVQWKVDNALQ | |
| | | NSGALTSGVHTFPAVLQSSGL | | SGNSQESVTEQDSKDSTYS | |
| | | YSLSSVVTVPSSSLGTQTYIC | | LSSTLTLSKADYEKHKVYA | |
| | | NVNHKPSNTKVDKKVEPKSCD | | CEVTHQGLSSPVTKSFNRG | |
| | | KTHTCPPCPAPELLGGPSVFL | | EC | |
| | | FPPKPKDTLMISRTPEVTCVV | | | |
| | | VDVSHEDPEVKFNWYVDGVEV | | | |
| | | HNAKTKPREEQYNSTYRVVSV | | | |
| | | LTVLHQDWLNGKEYKCKVSNK | | | |
| | | ALPAPIEKTISKAKGQPREPQ | | | |
| | | VYTLPPSRDELTKNQVSLTCL | | | |
| | | VKGFYPSDIAVEWESNGQPEN | | | |
| | | NYKTTPPVLDSDGSFFLYSKL | | | |
| | | TVDKSRWQQGNVFSCSVMHEA | | | |
| | | LHNHYTQKSLSLSPGK | | | |
| IgGC05 | 83 | EVQLVESGGGLVQPGGSLRLS | 220 | MTQSPSSLSASVGDRVTIT | WO2022212876 |
| | | CAASGFDFSSYSIHWVRQAPG | | CRASQSVSSAVAWYQQKPG | |
| | | KGLEWVASIYPYSGSTYYADS | | KAPKLLIYSASSLYSGVPS | |
| | | VKGRFTISADTSKNTAYLQMN | | RFSGSRSGTDFTLTISSLQ | |
| | | SLRAEDTAVYYCARQSHSYYS | | PEDFATYYCQQSRQRPLTF | |
| | | HTWWVSYGMDYWGQGTLVTVS | | GQGTKVEIKRTVAAPSVFI | |
| | | SASTKGPSVFPLAPSSKSTSG | | FPPSDSQLKSGTASVVCLL | |
| | | GTAALGCLVKDYFPEPVTVSW | | NNFYPREAKVQWKVDNALQ | |
| | | NSGALTSGVHTFPAVLQSSGL | | SGNSQESVTEQDSKDSTYS | |
| | | YSLSSVVTVPSSSLGTQTYIC | | LSSTLTLSKADYEKHKVYA | |
| | | NVNHKPSNTKVDKKVEPKSCD | | CEVTHQGLSSPVTKSFNRG | |
| | | KTHTCPPCPAPELLGGPSVFL | | EC | |
| | | FPPKPKDTLMISRTPEVTCVV | | | |
| | | VDVSHEDPEVKFNWYVDGVEV | | | |
| | | HNAKTKPREEQYNSTYRVVSV | | | |
| | | LTVLHQDWLNGKEYKCKVSNK | | | |
| | | ALPAPIEKTISKAKGQPREPQ | | | |
| | | VYTLPPSRDELTKNQVSLTCL | | | |
| | | VKGFYPSDIAVEWESNGQPEN | | | |
| | | NYKTTPPVLDSDGSFFLYSKL | | | |
| | | TVDKSRWQQGNVFSCSVMHEA | | | |
| | | LHNHYTQKSLSLSPGK | | | |
| IgGC06 | 84 | EVQLVESGGGLVQPGGSLRLS | 221 | MTQSPSSLSASVGDRVTIT | WO2022212876 |
| | | CAASGFDFSSYSIHWVRQAPG | | CRASQSVSSAVAWYQQKPG | |
| | | KGLEWVASIYPYSGSTYYADS | | KAPKLLIYSASSLYSGVPS | |
| | | VKGRFTISADTSKNTAYLQMN | | RFSGSRSGTDFTLTISSLQ | |
| | | SLRAEDTAVYYCARQSHSYYS | | PEDFATYYCQQSGLMPLTF | |
| | | HTWWVSYGMDYWGQGTLVTVS | | GQGTKVEIKRTVAAPSVFI | |
| | | SASTKGPSVFPLAPSSKSTSG | | FPPSDSQLKSGTASVVCLL | |
| | | GTAALGCLVKDYFPEPVTVSW | | NNFYPREAKVQWKVDNALQ | |
| | | NSGALTSGVHTFPAVLQSSGL | | SGNSQESVTEQDSKDSTYS | |
| | | YSLSSVVTVPSSSLGTQTYIC | | LSSTLTLSKADYEKHKVYA | |
| | | NVNHKPSNTKVDKKVEPKSCD | | CEVTHQGLSSPVTKSFNRG | |
| | | KTHTCPPCPAPELLGGPSVFL | | EC | |
| | | FPPKPKDTLMISRTPEVTCVV | | | |
| | | VDVSHEDPEVKFNWYVDGVEV | | | |
| | | HNAKTKPREEQYNSTYRVVSV | | | |
| | | LTVLHQDWLNGKEYKCKVSNK | | | |
| | | ALPAPIEKTISKAKGQPREPQ | | | |
| | | VYTLPPSRDELTKNQVSLTCL | | | |
| | | VKGFYPSDIAVEWESNGQPEN | | | |
| | | NYKTTPPVLDSDGSFFLYSKL | | | |
| | | TVDKSRWQQGNVFSCSVMHEA | | | |
| | | LHNHYTQKSLSLSPGK | | | |
| IgGC09 | 85 | EVQLVESGGGLVQPGGSLRLS | 222 | MTQSPSSLSASVGDRVTIT | WO2022212876 |
| | | CAASGFDFSSYSIHWVRQAPG | | CRASQSVSSAVAWYQQKPG | |
| | | KGLEWVASIYPYSGSTYYADS | | KAPKLLIYSASSLYSGVPS | |
| | | VKGRFTISADTSKNTAYLQMN | | RFSGSRSGTDFTLTISSLQ | |
| | | SLRAEDTAVYYCARQSHSYYS | | PEDFATYYCQQSGHRPITF | |
| | | HTWWVSYGMDYWGQGTLVTVS | | GQGTKVEIKRTVAAPSVFI | |
| | | SASTKGPSVFPLAPSSKSTSG | | FPPSDSQLKSGTASVVCLL | |
| | | GTAALGCLVKDYFPEPVTVSW | | NNFYPREAKVQWKVDNALQ | |
| | | NSGALTSGVHTFPAVLQSSGL | | SGNSQESVTEQDSKDSTYS | |
| | | YSLSSVVTVPSSSLGTQTYIC | | LSSTLTLSKADYEKHKVYA | |
| | | NVNHKPSNTKVDKKVEPKSCD | | CEVTHQGLSSPVTKSFNRG | |
| | | KTHTCPPCPAPELLGGPSVFL | | EC | |
| | | FPPKPKDTLMISRTPEVTCVV | | | |
| | | VDVSHEDPEVKFNWYVDGVEV | | | |
| | | HNAKTKPREEQYNSTYRVVSV | | | |
| | | LTVLHQDWLNGKEYKCKVSNK | | | |
| | | ALPAPIEKTISKAKGQPREPQ | | | |
| | | VYTLPPSRDELTKNQVSLTCL | | | |
| | | VKGFYPSDIAVEWESNGQPEN | | | |
| | | NYKTTPPVLDSDGSFFLYSKL | | | |
| | | TVDKSRWQQGNVFSCSVMHEA | | | |
| | | LHNHYTQKSLSLSPGK | | | |
| IgGD02 | 86 | EVQLVESGGGLVQPGGSLRLS | 223 | MTQSPSSLSASVGDRVTIT | WO2022212876 |
| | | CAASGFDFSSYSIHWVRQAPG | | CRASQSVSSAVAWYQQKPG | |
| | | KGLEWVASIYPYSGSTYYADS | | KAPKLLIYSASSLYSGVPS | |
| | | VKGRFTISADTSKNTAYLQMN | | RFSGSRSGTDFTLTISSLQ | |
| | | SLRAEDTAVYYCARKYHSYYS | | PEDFATYYCQQSSQRPATF | |
| | | HTWWVSYGMDYWGQGTLVTVS | | GQGTKVEIKRTVAAPSVFI | |
| | | SASTKGPSVFPLAPSSKSTSG | | FPPSDSQLKSGTASVVCLL | |
| | | GTAALGCLVKDYFPEPVTVSW | | NNFYPREAKVQWKVDNALQ | |
| | | NSGALTSGVHTFPAVLQSSGL | | SGNSQESVTEQDSKDSTYS | |
| | | YSLSSVVTVPSSSLGTQTYIC | | LSSTLTLSKADYEKHKVYA | |
| | | NVNHKPSNTKVDKKVEPKSCD | | CEVTHQGLSSPVTKSFNRG | |
| | | KTHTCPPCPAPELLGGPSVFL | | EC | |
| | | FPPKPKDTLMISRTPEVTCVV | | | |
| | | VDVSHEDPEVKFNWYVDGVEV | | | |
| | | HNAKTKPREEQYNSTYRVVSV | | | |
| | | LTVLHQDWLNGKEYKCKVSNK | | | |
| | | ALPAPIEKTISKAKGQPREPQ | | | |
| | | VYTLPPSRDELTKNQVSLTCL | | | |
| | | VKGFYPSDIAVEWESNGQPEN | | | |
| | | NYKTTPPVLDSDGSFFLYSKL | | | |
| | | TVDKSRWQQGNVFSCSVMHEA | | | |
| | | LHNHYTQKSLSLSPGK | | | |
| IgGD03 | 87 | EVQLVESGGGLVQPGGSLRLS | 224 | MTQSPSSLSASVGDRVTIT | WO2022212876 |
| | | CAASGFDFSSYSIHWVRQAPG | | CRASQSVSSAVAWYQQKPG | |
| | | KGLEWVASIYPYSGSTYYADS | | KAPKLLIYSASSLYSGVPS | |
| | | VKGRFTISADTSKNTAYLQMN | | RFSGSRSGTDFTLTISSLQ | |
| | | SLRAEDTAVYYCARQDHSYYS | | PEDFATYYCQQSGRQPLTF | |
| | | HTWWVSYGMDYWGQGTLVTVS | | GQGTKVEIKRTVAAPSVFI | |
| | | SASTKGPSVFPLAPSSKSTSG | | FPPSDSQLKSGTASVVCLL | |
| | | GTAALGCLVKDYFPEPVTVSW | | NNFYPREAKVQWKVDNALQ | |
| | | NSGALTSGVHTFPAVLQSSGL | | SGNSQESVTEQDSKDSTYS | |
| | | YSLSSVVTVPSSSLGTQTYIC | | LSSTLTLSKADYEKHKVYA | |
| | | NVNHKPSNTKVDKKVEPKSCD | | CEVTHQGLSSPVTKSFNRG | |
| | | KTHTCPPCPAPELLGGPSVFL | | EC | |
| | | FPPKPKDTLMISRTPEVTCVV | | | |
| | | VDVSHEDPEVKFNWYVDGVEV | | | |
| | | HNAKTKPREEQYNSTYRVVSV | | | |
| | | LTVLHQDWLNGKEYKCKVSNK | | | |
| | | ALPAPIEKTISKAKGQPREPQ | | | |
| | | VYTLPPSRDELTKNQVSLTCL | | | |
| | | VKGFYPSDIAVEWESNGQPEN | | | |
| | | NYKTTPPVLDSDGSFFLYSKL | | | |
| | | TVDKSRWQQGNVFSCSVMHEA | | | |
| | | LHNHYTQKSLSLSPGK | | | |
| IgGD04 | 88 | EVQLVESGGGLVQPGGSLRLS | 225 | MTQSPSSLSASVGDRVTIT | WO2022212876 |
| | | CAASGFDESSYSIHWVRQAPG | | CRASQSVSSAVAWYQQKPG | |
| | | KGLEWVASIYPYSGSTYYADS | | KAPKLLIYSASSLYSGVPS | |
| | | VKGRFTISADTSKNTAYLQMN | | RFSGSRSGTDFTLTISSLQ | |
| | | SLRAEDTAVYYCARRSHSYYS | | PEDFATYYCQQFMRPAFTF | |
| | | HTWWVSYGMDYWGQGTLVTVS | | GQGTKVEIKRTVAAPSVFI | |
| | | SASTKGPSVFPLAPSSKSTSG | | FPPSDSQLKSGTASVVCLL | |
| | | GTAALGCLVKDYFPEPVTVSW | | NNFYPREAKVQWKVDNALQ | |
| | | NSGALTSGVHTFPAVLQSSGL | | SGNSQESVTEQDSKDSTYS | |
| | | YSLSSVVTVPSSSLGTQTYIC | | LSSTLTLSKADYEKHKVYA | |
| | | NVNHKPSNTKVDKKVEPKSCD | | CEVTHQGLSSPVTKSFNRG | |
| | | KTHTCPPCPAPELLGGPSVFL | | EC | |
| | | FPPKPKDTLMISRTPEVTCVV | | | |
| | | VDVSHEDPEVKFNWYVDGVEV | | | |
| | | HNAKTKPREEQYNSTYRVVSV | | | |
| | | LTVLHQDWLNGKEYKCKVSNK | | | |
| | | ALPAPIEKTISKAKGQPREPQ | | | |
| | | VYTLPPSRDELTKNQVSLTCL | | | |
| | | VKGFYPSDIAVEWESNGQPEN | | | |
| | | NYKTTPPVLDSDGSFFLYSKL | | | |
| | | TVDKSRWQQGNVFSCSVMHEA | | | |
| | | LHNHYTQKSLSLSPGK | | | |
| IgGD07 | 89 | EVQLVESGGGLVQPGGSLRLS | 226 | MTQSPSSLSASVGDRVTIT | WO2022212876 |
| | | CAASGFDFSSYSIHWVRQAPG | | CRASQSVSSAVAWYQQKPG | |
| | | KGLEWVASIYPYSGSTYYADS | | KAPKLLIYSASSLYSGVPS | |
| | | VKGRFTISADTSKNTAYLQMN | | RFSGSRSGTDFTLTISSLQ | |
| | | SLRAEDTAVYYCARRSHSYYS | | PEDFATYYCQQSGQPSMTF | |
| | | HTWWVSYGMDYWGQGTLVTVS | | GQGTKVEIKRTVAAPSVFI | |
| | | SASTKGPSVFPLAPSSKSTSG | | FPPSDSQLKSGTASVVCLL | |
| | | GTAALGCLVKDYFPEPVTVSW | | NNFYPREAKVQWKVDNALQ | |
| | | NSGALTSGVHTFPAVLQSSGL | | SGNSQESVTEQDSKDSTYS | |
| | | YSLSSVVTVPSSSLGTQTYIC | | LSSTLTLSKADYEKHKVYA | |
| | | NVNHKPSNTKVDKKVEPKSCD | | CEVTHQGLSSPVTKSFNRG | |
| | | KTHTCPPCPAPELLGGPSVFL | | EC | |
| | | FPPKPKDTLMISRTPEVTCVV | | | |
| | | VDVSHEDPEVKFNWYVDGVEV | | | |
| | | HNAKTKPREEQYNSTYRVVSV | | | |
| | | LTVLHQDWLNGKEYKCKVSNK | | | |
| | | ALPAPIEKTISKAKGQPREPQ | | | |
| | | VYTLPPSRDELTKNQVSLTCL | | | |
| | | VKGFYPSDIAVEWESNGQPEN | | | |
| | | NYKTTPPVLDSDGSFFLYSKL | | | |
| | | TVDKSRWQQGNVFSCSVMHEA | | | |
| | | LHNHYTQKSLSLSPGK | | | |
| IgGD08 | 90 | EVQLVESGGGLVQPGGSLRLS | 227 | MTQSPSSLSASVGDRVTIT | WO2022212876 |
| | | CAASGFDFSSYSIHWVRQAPG | | CRASQSVSSAVAWYQQKPG | |
| | | KGLEWVASIYPYSGSTYYADS | | KAPKLLIYSASSLYSGVPS | |
| | | VKGRFTISADTSKNTAYLQMN | | RFSGSRSGTDFTLTISSLQ | |
| | | SLRAEDTAVYYCARQSHSYYS | | PEDFATYYCQQSVLRPFTF | |
| | | HTWWVSYGMDYWGQGTLVTVS | | GQGTKVEIKRTVAAPSVFI | |
| | | SASTKGPSVFPLAPSSKSTSG | | FPPSDSQLKSGTASVVCLL | |
| | | GTAALGCLVKDYFPEPVTVSW | | NNFYPREAKVQWKVDNALQ | |
| | | NSGALTSGVHTFPAVLQSSGL | | SGNSQESVTEQDSKDSTYS | |
| | | YSLSSVVTVPSSSLGTQTYIC | | LSSTLTLSKADYEKHKVYA | |
| | | NVNHKPSNTKVDKKVEPKSCD | | CEVTHQGLSSPVTKSFNRG | |
| | | KTHTCPPCPAPELLGGPSVFL | | EC | |
| | | FPPKPKDTLMISRTPEVTCVV | | | |
| | | VDVSHEDPEVKFNWYVDGVEV | | | |
| | | HNAKTKPREEQYNSTYRVVSV | | | |
| | | LTVLHQDWLNGKEYKCKVSNK | | | |
| | | ALPAPIEKTISKAKGQPREPQ | | | |
| | | VYTLPPSRDELTKNQVSLTCL | | | |
| | | VKGFYPSDIAVEWESNGQPEN | | | |
| | | NYKTTPPVLDSDGSFFLYSKL | | | |
| | | TVDKSRWQQGNVFSCSVMHEA | | | |
| | | LHNHYTQKSLSLSPGK | | | |
| IgGD09 | 91 | EVQLVESGGGLVQPGGSLRLS | 228 | MTQSPSSLSASVGDRVTIT | WO2022212876 |
| | | CAASGFNFSSSSIHWVRQAPG | | CRASQSVSSAVAWYQQKPG | |
| | | KGLEWVASIYPYSGSTSYADS | | KAPKLLIYSASSLYSGVPS | |
| | | VKGRFTISADTSKNTAYLQMN | | RFSGSRSGTDFTLTISSLQ | |
| | | SLRAEDTAVYYCARIYWVDFW | | PEDFATYYCQQSSSSLITE | |
| | | YGHFSYYRPALDYWGQGTLVT | | GQGTKVEIKRTVAAPSVFI | |
| | | VSSASTKGPSVFPLAPSSKST | | FPPSDSQLKSGTASVVCLL | |
| | | SGGTAALGCLVKDYFPEPVTV | | NNFYPREAKVQWKVDNALQ | |
| | | SWNSGALTSGVHTFPAVLQSS | | SGNSQESVTEQDSKDSTYS | |
| | | GLYSLSSVVTVPSSSLGTQTY | | LSSTLTLSKADYEKHKVYA | |
| | | ICNVNHKPSNTKVDKKVEPKS | | CEVTHQGLSSPVTKSFNRG | |
| | | CDKTHTCPPCPAPELLGGPSV | | E | |
| | | FLFPPKPKDTLMISRTPEVTC | | | |
| | | VVVDVSHEDPEVKFNWYVDGV | | | |
| | | EVHNAKTKPREEQYNSTYRVV | | | |
| | | SVLTVLHQDWLNGKEYKCKVS | | | |
| | | NKALPAPIEKTISKAKGQPRE | | | |
| | | PQVYTLPPSRDELTKNQVSLT | | | |
| | | CLVKGFYPSDIAVEWESNGQP | | | |
| | | ENNYKTTPPVLDSDGSFFLYS | | | |
| | | KLTVDKSRWQQGNVFSCSVMH | | | |
| | | EALHNHYTQKSLSLSPGK | | | |
| 10D7 | 92 | EVQLQQFGAELVKPGASVKIS | 229 | NIVMTQSPQSMSMSVGERV | US20220389113A1 |
| | | CKASGYSFSDFNIEWLKQSHG | | TLSCKASENVGAYVSWFQQ | |
| | | KSLEWIGDINPNYDSTNYNQK | | KPDQSPKLLILAASNRYTG | |
| | | FKGRATLTVDKSSSTAYMEVR | | VPARFIGSGSATDFTLTIS | |
| | | SLTSEDTAVYYCARLGYGYAM | | SVQAEDLADYHCGQSYTYP | |
| | | DYWGQGTSVTVSS | | YTFGGGTKLEIKRADAAPT | |
| | | | | VS | |
| Chimeric | 93 | EVQLQQFGAELVKPGASVKIS | 230 | IVMTQSPQSMSMSVGERVT | US20220389113A2 |
| 10D7 | | CKASGYSFSDFNIEWLKQSHG | | LSCKASENVGAYVSWFQQK | |
| | | KSLEWIGDINPNYDSTNYNQK | | PDQSPKLLILAASNRYTGV | |
| | | FKGRATLTVDKSSSTAYMEVR | | PARFIGSGSATDFTLTISS | |
| | | SLTSEDTAVYYCARLGYGYAM | | VQAEDLADYHCGQSYTYPY | |
| | | DYWGQGTSVTVSSASTKGPSV | | TFGGGTKLEIKRADAAPTV | |
| | | FPLAPSSKSTSGGTAALGCLV | | SIFPPSDEQLKSGTASVVC | |
| | | KDYFPEPVTVSWNSGALTSGV | | LLNNFYPREAKVQWKVDNA | |
| | | HTFPAVLQSSGLYSLSSVVTV | | LQSGNSQESVTEQDSKDST | |
| | | PSSSLGTQTYICNVNHKPSNT | | YSLSSTLTLSKADYEKHKV | |
| | | KVDKKVEPKSCDKTHTCPPCP | | YACEVTHQGLSSPVTKSEN | |
| | | APELLGGPSVFLFPPKPKDTL | | RGEC | |
| | | MISRTPEVTCVVVDVSHEDPE | | | |
| | | VKFNWYVDGVEVHNAKTKPRE | | | |
| | | EQYNSTYRVVSVLTVLHQDWL | | | |
| | | NGKEYKCKVSNKALPAPIEKT | | | |
| | | ISKAKGQPREPQVYTLPPSRE | | | |
| | | EMTKNQVSLTCLVKGFYPSDI | | | |
| | | AVEWESNGQPENNYKTTPPVL | | | |
| | | DSDGSFFLYSKLTVDKSRWQQ | | | |
| | | GNVFSCSVMHEALHNHYTQKS | | | |
| | | LSLSPGK | | | |
| Mab | 94 | QVTLKESGPGILQPSQTLSLT | 231 | DVLMTQTPLSLPVSLGDQA | US20110070246A1 |
| CDCP1- | | CSFSGFSLSTAGVGVSWIRQP | | SISCRSSQNIVHSYGNTYL | |
| 004 | | SGKGLEWLAHIYWDDDKRYNP | | EWYLQKPGQSPKLLIYKVS | |
| | | SLKSRLTISKDTSRNQVFLKI | | NRFSGVPDRFSGSGSGTDF | |
| | | TSVDSADTATYFCSRSTSVEE | | TLKISRVEAEDLGVYYCFQ | |
| | | AFDVWGAGTTVTVSS | | GSHVPWTFGGGTKLEIK | |
| Mab | 95 | QVTLKESGPGILQSSQTLSLT | 232 | DVVMTQTPLTLSVTIGQPA | US20110070246A1 |
| CDCP1- | | CSFSGFSLSTAGMGVSWIRQP | | SISCKSSQSLLYTNGKTYL | (LAROCHE) |
| 012 | | SGKGLEWLAHIYWDDDKRYNP | | IWLLQRPGQSPKRLIYLVS | |
| | | SLKSRLTVSKGTSRNQVLLKI | | KLDSGVPDRFSGSGSGTDF | |
| | | TSVDAADTATYYCARSDSFGD | | TLKISRVEAEDLGVYYCLQ | |
| | | FDYWGQGTTLTVSS | | STHFPYTFGGGTKLEIK | |
| Mab | 96 | QVHLKQSGPGLVQPSQSLSIT | 233 | DVVLTQTPLSLPVIIGDQA | US20110070246A1 |
| CDCP1- | | CTVSGFSLTNFGLHWVRQSPG | | SISCKSTKSLLNSAGFTYL | (LAROCHE) |
| 015 | | KGLEWLGVIWSGGVTDYNAAF | | GWYLQKPGQSPQLLIYLVS | |
| | | ISRLSISKDNSKSQVFFKMNS | | NRFSGVPDRFSGSGSGTDF | |
| | | LQPNDTAIYYCARNYDHDYSM | | TLKISRVEAEDLGVYFCFQ | |
| | | DYWGQGISVSVSS | | SSYLPLTFGSGTKLEIK | |
| CP13E10 | 97 | EVQLVQSGAEVKKPGASVKVS | 234 | EIVLTQSPATLSLSPGERA | US2022011954A1 |
| | | CKASGYTFTSYYMHWVRQAPG | | TLSCRASQSVGSYLAWYQQ | |
| | | QGLEWMGIINPSGGSTSYAQK | | RPGQAPRLLIYDASNRATG | |
| | | FQGRVTMTRDTSTSTVYMELS | | IPARFSGSGSGTDFTLTIS | |
| | | SLRSEDTAVYYCARDGVLRYF | | SLEPEDFAVYYCQQRANVF | |
| | | DWLLDYYYYMDVWGKGTTVTV | | TFGQGTKVEIK | |
| | | SS | | | |
| CP13E10- | 98 | EVQLVQSGAEVKKPGASVKVS | 235 | EIVLTQSPATLSLSPGERA | US2022011954A1 |
| 54HC- | | CKASGYTFTSYYMHWVRQAPG | | TLSCRASQSVGSYLAWYQQ | |
| 89LC | | QGLEWMGIINPSGGSTSYAQK | | RPGQAPRLLIYDASNRATG | |
| | | FQGRVTMTRDTSTSTVYMELS | | IPARFSGSGSGTDFTLTIS | |
| | | SLRSEDTAVYYCARDGELRHF | | SLEPEDFAVYYCQQRAQEF | |
| | | DHLLDYHYYMDVWGQGTTVTV | | TFGQGTKVEIK | |
| | | SS | | | |
| CP13E10- | 99 | EVQLVQSGAEVKKPGASVKVS | 236 | EIVMTQSPATLSLSPGERA | US2022011954A1 |
| 54HC- | | CKASGYTFTSYYMHWVRQAPG | | TLSCRASQSVGSYLAWYQQ | |
| 89LCv1 | | QGLEWMGIINPSGGSTSYAQK | | KPGQAPRLLIYDASNRATG | |
| | | FQGRVTMTRDTSTSTVYMELS | | IPARFSGSGSGTDFTLTIS | |
| | | SLRSEDTAVYYCARDGELRHF | | SLQPEDFAVYYCQQRAQEF | |
| | | DHLLDYHYYMDVWGQGTTVTV | | TFGQGTKVEIK | |
| | | SS | | | |
| CP13E10- | 100 | EVQLVQSGAEVKKPGASVKVS | 237 | EIVMTQSPATLSLSPGERA | US2022011954A1 |
| 54HCv13- | | CKASGYTFTSYYMHWVRQAPG | | TLSCRASQSVGSYLAWYQQ | |
| 89LC1 | | QGLEWMGIINPSGGSTSYAQK | | KPGQAPRLLIYDASNRATG | |
| | | FQGRVTMTRDTSTSTVYMELS | | IPARFSGSGSGTDFTLTIS | |
| | | SLRSEDTAVYYCARDAELRHF | | SLQPEDFAVYYCQQRAQEF | |
| | | DHLLDYHYYMDVWGQGTTVTV | | TFGQGTKVEIK | |
| | | SS | | | |
| CP13E10- | 101 | EVQLVQSGAEVKKPGASVKVS | 238 | EIVLTQSPATLSLSPGERA | US2022011954A1 |
| 291 | | CKASGYTFTSYYMHWVRQAPG | | TLSCRASQSVGSYLAWYQQ | |
| | | QGLEWMGIINPSGGSTSYAQK | | RPGQAPRLLIYDASNRATG | |
| | | FQGRVTMTRDTSTSTVYMELS | | IPARFSGSGSGTDFTLTIS | |
| | | SLRSEDTAVYYCARDGELRHF | | SLEPEDFAVYYCQQRANVF | |
| | | DQVYNYHYYMDVWGQGTTVTV | | TFGQGTKVEIK | |
| | | SS | | | |
| Antibody | 102 | EIVLTQSPATLSLSPGERATL | 239 | EIVLTQSPATLSLSPGERA | US2022011954A1 |
| 23 | | SCRASQSVGSYLAWYQQRPGQ | | TLSCRASQSVGSYLAWYQQ | |
| | | APRLLIYDASNRATGIPARFS | | RPGQAPRLLIYDASNRATG | |
| | | GSGSGTDFTLTISSLEPEDFA | | IPARFSGSGSGTDFTLTIS | |
| | | VYYCQQRANVFTFGQGTKVEI | | SLEPEDFAVYYCQQRANVF | |
| | | K | | TFGQGTKVEIK | |
| Antibody | 103 | QVQLVESGGGVVQPGRSLRLS | 240 | EIVMTQSPDTLSLSPGERA | US2022011954A1 |
| 24 | | CAASGFTFSSYGMHWVRQAPG | | TLSCRASQKIYYSYLAWYQ | |
| | | KGLEWVAVISYDGSNKYYADS | | QKPGQAPRLLISGASTRAS | |
| | | VKGRFTISRDNSKNTLYLQMN | | DISDRFSGGGSGTDFTLTI | |
| | | SLRAEDTAVYYCARGVGLPDI | | NSLESEDAAVYYCQQYDSL | |
| | | WGQGTMVTVSS | | PVTFGRGTKLEIK | |
| Antibody | 104 | QVQLVQSGAEVKKPGSSVKVS | 241 | SYELTQPPSVSVSPGQTAR | US2022011954A1 |
| 76 | | CKASGGTFSSYAISWVRQAPG | | ITCSGDALPKKYAFWYQQK | |
| | | QGLEWMGGIIPIFGTANYAQK | | SGQAPVLVIYEDSKRPSGI | |
| | | FQGRVTITADKSTSTAYMELS | | PEKFSGSSSGTMATLTISG | |
| | | SLRSEDTAVYYCARDPTYFDW | | AQVEDEADYYCYSTDSSDN | |
| | | TRRGYYYMDVWGKGTTVTVSS | | PRGVFGGGTKLTVL | |
| 14H4VH | 105 | EVQLLESGGDLVQPGGSLRLS | 242 | DIQMTQSPSSLSASVGDRV | US2022011954A1 |
| | | CAASGFTFNSYAMSWVRQAPG | | TITCRASQGISIYLAWFQQ | |
| | | KGLEWVSVLSGSGGDIHYADS | | KPGKVPKLLIYAASTLQSG | |
| | | VKGRFTVSRDNSKNMLYLQMN | | VPSRFSGSGSGTDFTLTIS | |
| | | SLRAEDTAVYFCAQQWPQGYW | | SLQPEDVATYYCQKYNSAP | |
| | | GQGTLVTVSS | | FTFGPGTKVEIK | |
| 03B05VH | 106 | QVQLVESGGGVVQPGRSLRLS | 243 | EIVMTQSPATLSVSPGERA | US2022011954A1 |
| | | CAASGFTFSSYGMHWVRQAPG | | TLSCRASQSVSSNLAWYQQ | |
| | | KGLEWVAIIWYDGSNKYYADS | | KPGQAPRLLIYGASTRATG | |
| | | VKGRFTISRDNSKNTLYLQMN | | IPARFSGSGSGTEFTLTIS | |
| | | SLRAEDTAVYYCAKDPSYSSG | | SLQSEDFAVYYCQQYNNWP | |
| | | YYLFDFWGQGTLVTVSS | | LTFGGGTKVEIK | |
| 03C05 | 107 | EVQVLESGGGLVQPGGSLRLS | 244 | DIQMTQSPSSLSASVGDRV | US2022011954A1 |
| | | CAASGFTFSSYVMSWVRQAPG | | TITCRASQDISIYLAWFQQ | |
| | | KGLEWVSGISGSGGSTHYADS | | KPGKVPKVLIYAASTLQSG | |
| | | VKGRFTISRDNSKNTLYLQMN | | VPSRFSGSGTGTDFTLTIS | |
| | | SLRAEDTALYYCAQQWPQGYW | | SLQPEDVATYFCQKYNSAP | |
| | | GQGTLVTVSS | | FTFGPGTKLEIK | |
| 27H10 | 108 | QVQLVQSGAEVKKPGSSVKVS | 245 | DIQMTQSPSSLSASVGDRV | US2022011954A1 |
| | | CKASGGTFSSYAMSWVRQAPG | | TITCRASQSISSYLNWYQQ | |
| | | QGLEWMGGIIPILGTTNYAQK | | KPGKAPKFLIYVASSLQSG | |
| | | FQGRVTITADKSTSTAYMELS | | VPSRFSGRGSGTDFTLTIS | |
| | | SLRSEDTAVYYCAREGLYAFD | | SLQPEDFATYYCQQSYSTP | |
| | | IWGQGTMVTVSS | | WTFGQGTKVEIK | |
| 03B11 | 109 | QVQLQQWDAGLLKPSETLSLT | 246 | DIQLTQSPSFLSASVGDRV | US2022011954A1 |
| | | CAVYGGSFSSYYWSWIRQPPG | | TITCRASQGISSYLAWYQQ | |
| | | KGLEWIGEINHSGSTSYNPSL | | KPGKAPKLLIYAASTLQSG | |
| | | KSRVTISIDTSKNQFSLKINS | | VPSRFSGSGSGTEFTLTIS | |
| | | MTAADTAVYFCAASPYFDYWG | | SLQPEDFATYYCQQLNSYP | |
| | | QGTLVTVSS | | RTFGQGTKVEIK | |
| 04G06 | 110 | QVQLVESGGGVVQPGRSLRLS | 247 | DIQLTQSPSFLSASVGDRV | US2022011954A1 |
| | | CVVSGFTLSSYGMHWVRQAPG | | TITCRASQDISNYLAWYQQ | |
| | | KGLEWVAVIWYDGSDKYYADS | | KPGKAPKLLISASTLQSGV | |
| | | VKGRFTISRDNSKNTLYLQMN | | PSRFSGSGSGTEFILTISS | |
| | | SLRAEDTAVYYCARDDWGFDY | | LQPEDFATYYCQHLNSYPF | |
| | | WGQGTLVTVSS | | GQGTKVEIK | |
| 42C5 | 111 | EVQLVESGGGLVQPGGSLRLS | 248 | DIVMTQSPLSLPVTPGEPA | US2022011954A1 |
| | | CAASGFTFSRYWMNWVRQAPG | | SISCRSSRSLLHSSGYNFL | |
| | | KGLEWVANIKQDGSEKDYVDS | | DWFLQKPGQSPQLLIFLGS | |
| | | VKGRFTISRDNAKNSLFLQMS | | DRASGVPDRFSGSGSGTDF | |
| | | SLRAEDTAVYYCARVMYSSGW | | TLKISRVETEDVGVYYCMQ | |
| | | SFDYWGQGTLVTVSS | | ALQTPITFGQGTRLEIK | |
| 47G7 | 112 | EVQLVESGGGLVQPGGSLRLS | 249 | DIVMTQSPLSLPVTPGEPA | US2022011954A1 |
| | | CAASGFTFSSYWMSWVRQAPG | | SISCRSSQSLLHSSGHNFL | |
| | | KGLEWVANIKQDGSEKDYVDS | | DWYLQKPGQSPQLLIYLGS | |
| | | VKGRFTISRDNAKNSLYLQMN | | NRASGVPDRFSGSGSGTDF | |
| | | SLRVEDTAVYYCAREGGSSGW | | TLKISRVEAEDVGVYYCMQ | |
| | | TFDYWGQGTLVTVSS | | ALQTPPTFGQGTKLEIK | |
| 18C06 | 113 | QVQLQESGPGQVKPSETLSLT | 250 | EIVMTQSPATLSVSPGERA | US2022011954A1 |
| | | CTVSGGSISSSFWSWIRQPPG | | TLSCRASQSVSSNLAWYQQ | |
| | | KGLEWIGYIYYSESTNYNPSL | | KPGQAPRLLIYGASTRATG | |
| | | KRRVTLSVDTSKNQFSLKLTS | | IPARFSGSGSGTEFTLTIS | |
| | | VTTADTAVYYCARNIGVAGLF | | SLQSEDFAVYYCQQYNNWP | |
| | | DYWGQGTLVTVSS | | LTFGGGTKVEIK | |
| 11F9 | 114 | QVQLQESGPGLVKPSETLSLT | 251 | EIVMTQSPATLSVSPGERA | US2022011954A1 |
| | | CTVSGGSISSYYWSWIRQPPG | | TLSCRASQSVSSNLAWYQQ | |
| | | KGLEWIGYIYYSGSTNYNPSL | | KPGQAPRLLIYGASTRATG | |
| | | KSRVTISVDTSKNQFSLKLSS | | IPARFSGSGSGTEFTLTIS | |
| | | VTAADTAVYYCARNIGVAGLF | | SLQSEDFAVYYCQQYNNWP | |
| | | DYWGQGTLVTVSS | | LTFGGGTKLEIK | |
| 10E2. | 115 | QVQLVESGGGVVQPGRSLRLS | 252 | DIQMTQSPSTLSASVGDRV | US2022011954A1 |
| | | CAASGFTFSSYGMHWVRQAPG | | TITCRASQSISTWLAWYQQ | |
| | | KGLEWVAVVWYDGTIKYYADS | | KPGKAPKFLIYKASSLESG | |
| | | VKGRFTISRDNPKNTLYLQMN | | VPSRFSGSGSGTEFTLTIS | |
| | | SLRAEDTAVYYCASQYSSGWH | | SLQPDDFATYYCQQYSGYS | |
| | | TDFFDVWGQGTMVTVSS | | LTFGGGTKVEIK | |
| 01E2. | 116 | QVQLVESGGGVVQPGRSLRLS | 253 | EIVLTQSPATLSLSPGERA | US2022011954A1 |
| | | CAASGFTFSSYGMHWVRQAPG | | TLSCRASQTVPNYLAWYQQ | |
| | | KGLEWVAVIWYAGSNKYYADS | | KPGQAPRLLIYDASNRATD | |
| | | VKGRFTISRDNSKNTLYLQMN | | IPARFSGSGSGTDFTLTIS | |
| | | SLRAEDTAVYYCAGWDFDYWG | | SLEPEDFAVYYCQQRANWP | |
| | | QGTLVTVSS | | PITFGQGTRLEIK | |
| 41A9 | 117 | QVQLQQWGAGLLKPSETLSLT | 254 | EIVLTQSPATLSLSPGERA | US2022011954A1 |
| | | CAVYGGSFSDYYWSWIRQPPG | | TLSCRASQSVRRYLAWYQQ | |
| | | KGLEWIGEINHSGSTNYNPSL | | KPGQAPRLLIYDASNRATG | |
| | | KSRVTISVDTSKNQFSLKLSS | | IPARFSGSGSGTDFTLTIS | |
| | | VTAADTAVYYCARDYDVLTGH | | SLEPEDFAVYYCQQRNNWP | |
| | | FYYYYGMDVWGQGTTVTVSS | | LTFGGGTKLEIK | |
| 02F3 | 118 | QVQLVESGGGVVQPGRSLRLS | 255 | DIVMTQSPLSLPVTPGEPA | US2022011954A1 |
| | | CAASGFSFSDYGIHWVRQAPG | | SISCRSSQSLLHSNGYNYL | |
| | | KGLEWVAVIWYDGSNKYYADS | | DWYLQKPGQSPQILIYLGS | |
| | | VKGRFTISRDNSKNTLYLQVN | | NRASGVPDRFSGSGSGTDF | |
| | | SLIAEDTAVYYCARDRGYSSG | | TLKISRVEAEDVGVYYCMQ | |
| | | WYVDYYYYGMDVWGQGTTVTV | | ALQTPWTFGQGTKLEIK | |
| | | SS | | | |
| 50D6 | 119 | EVQVLESGGGLVQPGGSLRLS | 256 | DIQLTQSPSFLSASVGDRV | US2022011954A1 |
| | | CAASGFTFSNYAMNWVRQAPG | | TITCRASQGISNYLAWYQQ | |
| | | KGLEWVSAISGGGGSTYYADS | | KPGKVPKLLIYAVSTLQSG | |
| | | VKGRATISRDNSENTLYLQMN | | VPSRFSGSGSGTEFTLTIS | |
| | | SLRAEDTAVYYCAKTSSGWYD | | SLQPEDFATYYCQQLNSYP | |
| | | SYYDYYGLDVWGQGTTVTVSS | | FTFGGGTKLEIK | |
| 38E11. | 120 | EVQLLESGGGLVQPGGSLRLS | 257 | DIQLTQSPSFLSASVGDRV | US2022011954A1 |
| | | CAASGFTFSNYAMNWVRQAPG | | TITCRASQGISSYLAWYQQ | |
| | | KGLEWVSAISGGGGSTYYADS | | KPGKAPKLLIYAASTLQGG | |
| | | VKGRFTISRDNSKNTLYLQMN | | VPSRFSGSGSGTEFTLTIS | |
| | | SLRAEDTAVYYCAKESITMVR | | SLQPEDFATYYCQHLNRFP | |
| | | GVMDYYGMDVWGQGTTVTVSS | | RTFGQGTKVEIK | |
| 05A8 | 121 | EVQLLESGGGLVQPGGSLRLS | 258 | EIVMTQSPATLSVSPGERA | US2022011954A1 |
| | | CAASGFTFSNYAMSWVRQAPG | | TLSCRASQSVSSNLAWYQQ | |
| | | KGLEWVSAISGRGGSTYYADS | | KPGQAPRLLIYGASTRATG | |
| | | VKGRFTISRDNSKNTLYLQMN | | IPARFSGSGSGTEFTLTFS | |
| | | SLRAEDTAVYYCAKDIVVVPA | | SLQSEDFVVYYCQQYNNWP | |
| | | AKGYVMDAWGQGASVTVSS | | LTFGQGTRLEIK | |
| 12C2 | 122 | QEQLVESGGGVVQPGRSLRLS | 259 | DILMTQSPSSLSASVGDRV | US2022011954A1 |
| | | CVTSGFTFSSYGMHWVRQAPG | | TITCRASQGINYYLAWYQQ | |
| | | KGLEWVAVIWYDGTNKYYADT | | KPGKVPKLLIYTASTLQSG | |
| | | VKGRFTISRDNSKNTLYLQMN | | VPSRFSGSGSGTDFTLTIS | |
| | | SLRAEDTGVYYCAREGCDTIS | | SLQPEDVATYFCQKYNSAP | |
| | | CPYYYYGMDVWGQGTTITVSS | | FTFGPGTKLEIK | |
| 03G11 | 123 | EVQLLESGGGLVQPGGSLRLS | 260 | EIVMTQSPATLSVSPGERG | US2022011954A1 |
| | | CAASGFTFSSYAMSWVRQAPG | | TLSCRASQSVSSNLAWYQQ | |
| | | KGLEWVSAISGSGNSAYYADS | | KPGQAPRLLIYGASIRATG | |
| | | VKGRFTISRDNSKNTLFLQMN | | IPARFSGSGSGTEFILTIN | |
| | | SLRADDTAVYYCASSSGWYLV | | SLQSEDFAVYYCQQYNNWP | |
| | | YYFDLWGRGTLVTVSS | | LTVGGGTKVEIK | |
| 06H1 | 124 | EVQLLESGGGLVQPGGSLRLS | 261 | DIQMTQSPSSLSASVGDRV | US2022011954A1 |
| | | CAASGFTFSSYAMSWVRQAPG | | TITCRASQGISIYLAWFQQ | |
| | | KGLEWVSGISGSGGNTHYADS | | KPGKVPKLLIYAASTLQSG | |
| | | VKGRFTISRDNSKNTLYLQMN | | VPSRFSGSGSGTDFTLTIS | |
| | | SLRAEDTALYYCAQQWPQGYW | | SLQPEDFATYYCQKYNSAP | |
| | | GQGTLVTVSS | | FTFGPGTKLEIK | |
| 07C06- | 125 | EVQLLESGGGLVQPGGSLGLS | 262 | DIQMTQSPSSLSASVGDRV | US2022011954A1 |
| D2 | | CAASGFTFSNYIMSWVRQAPG | | TITCRASQGITIYLAWFQQ | |
| | | KGLEWVSGISGSGGSTHYAGS | | KPGKVPKLLIYAASTLQSG | |
| | | VKGRFTISRDNSKNTLNLQMN | | VPSRFSGSGSGTDFTLTIS | |
| | | SLRVEDTAVYHCVQQWPQGYW | | SLQPEDVATYYCQKYNSAP | |
| | | GQGTLVTVSS | | FTFGPGTKLEIK | |
| 09F6 | 126 | EVQLLESGGGLVQPGGSLRLS | 263 | DIQMTQSPSSLSASVGDRV | US2022011954A1 |
| | | CAASGFTFSSYAMSWVRLAPG | | TITCRASQGISIYLAWFQQ | |
| | | KGLEWVSGLSGSGGDTHYAGS | | KPGKVPKLLIYAASTLQSG | |
| | | VKGRFTISRDNSKNTLYLQMN | | VPSRFSGSGSGTDFTLTIS | |
| | | SLRAEDTALYYCAQQWPQGYW | | SLQPEDVATYYCQKYNSAP | |
| | | GQGTLVTVSS | | FTFGPGTKLEIK | |
| 14D4 | 127 | EVQLLESGGGLVQPGESLRLS | 264 | DIQMTQSPSSLSASVGDRV | US2022011954A1 |
| | | CAASGFTFSSYAMSWVRQAPG | | TITCRASQDISIYLAWFQQ | |
| | | KGLEWVSGISGSGGSTHYADS | | RPGKVPKLLIYAASTLQSG | |
| | | VKGRFTISRDNSKNTLYLQMN | | VPSRFSGSGSGTDFTLTIS | |
| | | SLRAEDTAAYYCAQQWPQGHW | | SLQPEDVATYYCQKYNSDP | |
| | | GQGILVTVSS | | FTFGPGTKVEIK | |
| 15H5 | 128 | EVQLLESGGGLVQPGGSLRLS | 265 | DIQMTQSPSSLSASVGDRV | US2022011954A1 |
| | | CAASGFTFSSYAMSWVRQAPG | | TITCRASQDISIYLAWFHQ | |
| | | KGLEWVSVLSGSGDDTHYADS | | KPGKVPKLLIYAASTLQSG | |
| | | VKGRFTISRDNSKNTLYLQMN | | VPSRFSGSGSGTDFTLTIS | |
| | | SLRAEDTAVYYCAHQWPQGYW | | SLQPEDVAAYYCQKYNSAP | |
| | | GQGTLVTVSS | | FTFGPGTKLEIK | |
| 20F5 | 129 | EVQLLESGGGLVQPGGSLRLS | 266 | DIQMTQSPSSLSATVGDRV | US2022011954A1 |
| | | CAASGFTFSSYVMSWVRQTPG | | TITCRASQGISIYLAWFQQ | |
| | | KGLEWVSGISGSGGSTHYTDS | | KPGKVPKNLIYAASTLQSG | |
| | | VQGRFTISRDNSKNTLYLQMN | | VPSRFSGSGSGTDFTLTIS | |
| | | SLRAEDTAVYYCVQQWPQGYW | | SLQPEDFATYYCQKYNSAP | |
| | | GQGTLVTVSS | | FTFGPGTKLEIK | |
| 38A6 | 130 | EVQVLESGGGLVQPGESLRLS | 267 | DIQMTQSPSSLSASVGDRV | US2022011954A1 |
| | | CAASGFTFNTYAMSWVRQAPG | | TITCRASQGISIYLAWYHQ | |
| | | KGLEWVSAISDNGGGTYNADS | | KPGKVPKLLIYAASTLQSG | |
| | | VKGRFTISRDNSKNTLYLQMN | | VPSRFSGSGSGTDFTLTIS | |
| | | SLRAEDTAVYYCAKGEQWGAP | | SLQPEDVATYYCQKYNSAP | |
| | | FDYWGQGTLVTVSS | | WTFGQGTKLEIK | |
| 39D7 | 131 | EVQLLESGGGLVQPGGSLRLS | 268 | DIVMTQSPLSLPVTPGEPA | US2022011954A1 |
| | | CAASGFTFSSYAMSWVRQAPG | | SISCRSSQSLLHSNGYNYL | |
| | | KGLEWVSTISGSGGSTYYADS | | DWYLQKSGQSPQLLIYLGS | |
| | | VKGRFTISRDNSKNTLYLQMN | | NRASGVPDRFSGSGSGTDF | |
| | | SLRAEDTAVYYCATLDTAMAA | | TLKISRVEAEDVGVYYCMQ | |
| | | DAFAIWGQGTMVTVSS | | ALLIPLYTFGQGTKLEIK | |
| 18C11 | 132 | EVQLVESGGGLVQPGGSLRLS | 269 | DIVMTQSPLSLPVTPGEPA | US2022011954A1 |
| | | CAASGFTFSSYWMSWVRQVPG | | SISCRSSQSLLHSSGHNFL | |
| | | KGLEWVANIKQDGSEKYYVDS | | DWYLKKPGQSPQLLIYLGS | |
| | | VKGRFTISRDNAKNSLYLQMN | | NRGSGVPDRFSGSGSGTDF | |
| | | SLRAEDTAVYYCARDWRSSGW | | TLKISRVEAEDVGVYYCMQ | |
| | | TLDYWGQGTLVTVSS | | ALQTPRTFGQGTKLEIK | |
| 38G4 | 133 | EVQLVESGGGLVQPGGSLRLS | 270 | DIVMTQSPLSLPVTPGEPA | US2022011954A1 |
| | | CAASGFTFSSYWMSWVRQAPG | | SISCRSSQSLLHSTGYNFL | |
| | | KGLEWVANVKQDGSEKDYVDS | | DWYLQKPGQSPQLLIFLGS | |
| | | VKGRFTISRDNAKNSLYLQMN | | NRASGVPDRFSGSGSGTDF | |
| | | SLRAEDTAVYYCAREWNSSGW | | TLKISRVEAEDVGIYYCMQ | |
| | | TFDYWGQGTLVTVSS | | ALQTPLTFGGGTKVEIK | |
| 27D8 | 134 | QVQLVESGGGVVQPGRSLRLS | 271 | EIVMTQSPATLSVSPGERA | US2022011954A1 |
| | | CAASGFTFSSYGMHWVRQAPG | | TLSCRASQSVSNNLAWYQQ | |
| | | KGLEWVAVIWYDGSNKYYADS | | KPGQAPRLLIYGVSTRATG | |
| | | VKGRFTISRDNSKNTLYLQMN | | IPARFSGSGSGTEFTLIIS | |
| | | SLRAEDTAVYHCARDRVYYDG | | SLQSEDFAGYYCQQYNDWP | |
| | | SGSYYNVGVMDVWGQGASVTV | | LTFGGGTKLEIK | |
| | | SS | | | |
| 11A4 | 135 | QVQLVESGGGVVQPGGSPRLS | 272 | DIQMTQSPSTLSASVGDRV | US2022011954A1 |
| | | CAASGFTFSSYAMHWVRQAPG | | TITCRASQSISSWLAWYQQ | |
| | | TGLEWVALIYYDGSHEYYSDS | | KPGKAPNLLIYKASSLESG | |
| | | VKGRFTISRDNSKNTLYLQMS | | VPSRFSGSGSGTEFTLTIS | |
| | | SLRAEDTAVYYCARDGGSGSH | | SLQPDDFATYYCQQYNTYY | |
| | | YPFDAFDIWGQGTMVTVSS | | TFGQGTKVEIK | |
| 24H1 | 136 | QVQLQESGPGLVKPSETLSLT | 273 | DIVMTQSPLSLPVTPGEPA | US2022011954A1 |
| | | CTVSGGSISSYYWSWIRQFSG | | SISCRSSQSLLHSSGYNYL | |
| | | KGLEWIGYIYYTGRNNYNPSL | | DWYLQKPGQSPQLLIYLGS | |
| | | KSRVTISVDTSKNQFSLKLSS | | DRASGVPDRFRGSGSGTDF | |
| | | VTAADSAVYYCAREGGWGPHF | | TLKISRVEAEDVGIYYCMQ | |
| | | DYWGQGTLVTVSS | | ALQIPYTFGQGTKLEIK | |
| 18C02 | 137 | QVQLQQWGAGLLKPSETLSLT | 274 | DIQMTQSPSSLSASVGDRV | US2022011954A1 |
| | | CAVYGGSFSDYYWSWIRQPPG | | TITCRASQSISSYLNWFQQ | |
| | | KGLEWIGEINHSGSTNYNPSL | | KPGKAPKLLIYAASSLQSG | |
| | | KSRVTISVDTSKNQFSLKLSS | | VPSRFSGSGSGTDFTLTIS | |
| | | VTAADTAVYYCARDYDVLTGH | | SLQPEDFATYYCQQSYTMP | |
| | | FYYYYGMDVWGQGTTVTVSS | | YTFGQGTKLEIK | |
| 07C10 | 138 | QVQLVESGGGVVQPGRSLRLS | 275 | EIVLTQSPATLSLSPGERA | US2022011954A1 |
| | | CAASGFTFSSYGMHWVRQAPG | | TLSCRASQSVTNYLAWFQQ | |
| | | KGLEWVALIWYDGSNKYYADS | | KPGQAPRLLIYDAFNRATG | |
| | | VQGRFTISRDNSKNTLYLQMN | | IPARFSGSGSGTDFTLTIS | |
| | | SLRAEDTAVYYCAVDYDILTG | | SLEPEDFAIYYCQQRSYWP | |
| | | HVLYVMDAWGQGVSVTVSS | | LTFGGGTKLEIK | |
| 12D9 | 139 | QVQLVESGGGVVQPGRSLRLS | 276 | DIQLTQSPSFLSASVGDRV | US2022011954A1 |
| | | CAASGFTFSNYGMNWVRQAPG | | TITCRASQGISSYLAWYQQ | |
| | | KGLEWVAVIWYDGTIKYYADS | | KPGKAPKLLIYAASTLQSG | |
| | | VKGRFTISRDNSKNTLYLQMI | | VPSRFSGSGSGTEFTLTIS | |
| | | SLRAEDTAVYYCASEYSSGWY | | SLQPEDFATYYCQQLNSYP | |
| | | RGAFDIWGQGTMVTVSS | | YTFGQGTKVEIK | |
| 21F2 | 140 | QVQLVESGGGVVQPGRSLRLS | 277 | DIQMTQSPSTLSASVGDRV | US2022011954A1 |
| | | CAASGFTFSSYGMFWVRQTPG | | TITCRASQSISSWLAWYQQ | |
| | | KGLEWVANIWYDGSNKYYTDS | | KPGKAPKLLIYKASSLESG | |
| | | VKGRFTISRDNSKNTLYLQMN | | VPSRFSGSGSGTEFTLTIS | |
| | | SLRAGDTAVYYCARETYYYGS | | SLQPDDFATYYCQQYYSYI | |
| | | GSYGGGLDVWGQGTTVTVSS | | TFGQGTRLEIK | |
| 25B12 | 141 | QVQLQESGPGLVKPSETLSLT | 278 | DIQLTQSPSFLSASVGDRV | US2022011954A1 |
| | | CAVSGYSISSGYYWGWIRQPP | | TITCRASQGISSYLAWYQQ | |
| | | GKGLEWIGSIYHSGSTYYNPS | | KPGKAPKLLIYAASTLQSG | |
| | | LKSRVTISVDTSKNQFSLKLS | | VPSRFSGSGSGTEFTLTIS | |
| | | SVTAADTAVYYCARDKITVAA | | SLQPEDFATYYCQQLNSYP | |
| | | FDIWGQGTMVTVSS | | YTFGQGTKLEIK | |
| 47A2 | 142 | QVQLQESGPGLVKPSETLSLT | 279 | EIVLTQSPATLSLSPGERA | US2022011954A1 |
| | | CAVSGYSISSDYYWGWIRQPP | | TLSCRASQSVRSYLAWYQQ | |
| | | GKGLEWIGSIYHSGSTYYNTS | | KPGQAPRLLIYDASKRATG | |
| | | LKSRVTISLDTSKNQFSLKLT | | IPARFSGSGSGTDFTLTIS | |
| | | SVTAADTAVYYCVREGTVGGH | | SLEPEDFAVYYCQQRNSWP | |
| | | YYYYYGMDVWGQGTTVTVSS | | TFGGGTKVEIK | |
| 14F11 | 143 | EVHLLESGGGLVQPGGSLRLS | 280 | DIQLTQSPSFLSASVGDRV | US2022011954A1 |
| | | CAASGFTFSSYAMSWVRQAPG | | TITCRASQGISSYLAWYQQ | |
| | | KGLEWVSGIDSGGDTYYADSV | | KPGKAPKLLIYAASTLQSG | |
| | | KGRFTISRDNSKNTLYLQMTS | | VPSRFSGSGSGTEFTLTIS | |
| | | LRAEDTAVYYCAKDLYSSGWL | | SLQPEDFAIYYCQQLNSYP | |
| | | AFDIWGQGTMVTVSS | | LYTFGQGTKLEIK | |
| 18B2 | 144 | EVQLLESGGGSVQPGGSLRLS | 281 | DIQLTQSPSFLSASVGDRV | US2022011954A1 |
| | | CAASGFTFSSYAMSWVRQAPG | | TITCRASQGISTYLAWYQQ | |
| | | KGLEWVSGISDNGNTYYADSV | | KPGKAPKLLIYAASTLQSG | |
| | | KGRFTISRDNSKNTLYLQMNS | | VPSRFSGSGSGTEFTLTIS | |
| | | LRAEDTAVYHCAKDLYSSGWL | | SLQPEDFATYYCQQLNSYP | |
| | | AFDIWGQGTMVTVSS | | LYTFGQGTKVEIK | |
| 25A11 | 145 | VQLQQPGAELVKPGASVKMSC | 282 | DIQMTQTTSSLSASLGDRV | US20080008719A1 |
| | | KASGYTFTSYYMYWVKQRPGQ | | TISCRASQDISNYLNWYQQ | |
| | | GLEWIGEINPSHGGTNFNEKF | | KPDGTVKLLIYYTSRLHSG | |
| | | KNKATLTVDKSSSTVYMQLSS | | VPSRFSGSGSGTDYSLTIS | |
| | | LTSEDSAVYYCTRGGNYPYFA | | NLEQEDIATYFCQQGNTLP | |
| | | MDYWGQGTSVTVSS | | WT | |
| Chimeric | 146 | VQLQQPGAELVKPGASVKMSC | 283 | DIQMTQTTSSLSASLGDRV | US20080008719A1 |
| 25A11 | | KASGYTFTSYYMYWVKQRPGQ | | TISCRASQDISNYLNWYQQ | |
| | | GLEWIGEINPSHGGTNFNEKF | | KPDGTVKLLIYYTSRLHSG | |
| | | KNKATLTVDKSSSTVYMQLSS | | VPSRFSGSGSGTDYSLTIS | |
| | | LTSEDSAVYYCTRGGNYPYFA | | NLEQEDIATYFCQQGNTLP | |
| | | MDYWGQGTSVTVSSASTKGPS | | WTFGGGTKLEIKRTVAAPS | |
| | | VFPLAPSSKSTSGGTAALGCL | | VFIFPPSDEQLKSGTASVV | |
| | | VKDYFPEPVTVSWNSGALTSG | | CLLNNFYPREAKVQWKVDN | |
| | | VHTFPAVLQSSGLYSLSSVVT | | ALQSGNSQESVTEQDSKDS | |
| | | VPSSSLGTQTYICNVNHKPSN | | TYSLSSTLTLSKADYEKHK | |
| | | TKVDKRVEPKSCDKTHTCPPC | | VYACEVTHQGLSSPVTKSF | |
| | | PAPELLGGPSVFLFPPKPKDT | | NRGEC | |
| | | LMISRTPEVTCVVVDVSHEDP | | | |
| | | EVKFNWYVDGVEVHNAKTKPR | | | |
| | | EEQYNSTYRVVSVLTVLHQDW | | | |
| | | LNGKEYKCKVSNKALPAPIEK | | | |
| | | TISKAKGQPREPQVYTLPPSR | | | |
| | | EEMTKNQVSLTCLVKGFYPSD | | | |
| | | IAVEWESNGQPENNYKTTPPV | | | |
| | | LDSDGSFFLYSKLTVDKSRWQ | | | |
| | | QGNVFSCSVMHEALHNHYTQK | | | |
| | | SLSLSPGK | | | |
| hHC4- | 147 | EVQLVESGGGLVQPGGSLRLS | 284 | EVQLVESGGGLVQPGGSLR | U.S. Pat. No. |
| H- | | CAASGFTFNSYGMSWVRQAPG | | LSCAASGFTFNSYGMSWVR | 9346886 B2 |
| humanized | | KGLEWVATISSGGSYKYYVDS | | QAPGKGLEWVATISSGGSY | |
| | | VKGRFTISRDNAKNSLYLQMN | | KYYVDSVKGRFTISRDNAK | |
| | | SLRAEDTAVYYCARHPDYDGV | | NSLYLQMNSLRAEDTAVYY | |
| | | WFAYWGQGTLVTVSS | | CARHPDYDGVWFAYWGQGT | |
| | | | | LVTVSS | |
| hHC4- | 148 | EVQLVESGGGLVQPGGSLRLS | 285 | EVQLVESGGGLVQPGGSLR | U.S. Pat. No. |
| c- | | CAASGFTFNSYGMSWVRQAPG | | LSCAASGFTFNSYGMSWVR | 9346886 B2 |
| humanized | | KGLEWVATISSGGSYTYYPDS | | QAPGKGLEWVATISSGGSY | |
| | | VKGRFTISRDNAKNSLYLQMN | | TYYPDSVKGRFTISRDNAK | |
| | | SLRAEDTAVYYCARHPDYDGV | | NSLYLQMNSLRAEDTAVYY | |
| | | WFAYWGQGTLVTVSS | | CARHPDYDGVWFAYWGQGT | |
| | | | | LVTVSS | |
| hHC4- | 149 | EVQLVESGGGLVKPGGSLRLS | 286 | EVQLVESGGGLVKPGGSLR | U.S. Pat. No. |
| a- | | CAASGFTFNSYGMSWVRQAPG | | LSCAASGFTFNSYGMSWVR | 9346886 B2 |
| humanized | | KGLEWVSTISSGGSYTYYPDS | | QAPGKGLEWVSTISSGGSY | |
| | | VKGRFTISRDNAKNSLYLQMN | | TYYPDSVKGRFTISRDNAK | |
| | | SLRAEDTAVYYCARHPDYDGV | | NSLYLQMNSLRAEDTAVYY | |
| | | WFAYWGQGTLVTVSS | | CARHPDYDGVWFAYWGQGT | |
| | | | | LVTVSS | |
| hHC4- | 150 | EVQLLESGGGLVQPGGSLRLS | 287 | EVQLLESGGGLVQPGGSLR | U.S. Pat. No. |
| d- | | CAASGFTFNSYGMSWVRQAPG | | LSCAASGFTFNSYGMSWVR | 9346886 B2 |
| humanized | | KGLEWVSTISSGGSYTYYPDS | | QAPGKGLEWVSTISSGGSY | |
| | | VKGRFTISRDNSKNTLYLQMN | | TYYPDSVKGRFTISRDNSK | |
| | | SLRAEDTAVYYCAKHPDYDGV | | NTLYLQMNSLRAEDTAVYY | |
| | | WFAYWGQGTLVTVSS | | CAKHPDYDGVWFAYWGQGT | |
| | | | | LVTVSS | |
| hHC4- | 151 | EVQLLESGGGLVQPGGSLRLS | 288 | EVQLLESGGGLVQPGGSLR | U.S. Pat. No. |
| 04- | | CAASGFTFNSYGMSWVRQAPG | | LSCAASGFTFNSYGMSWVR | 9346886 B2 |
| humanized | | KGLEWVSTISSGGSYTYYPDS | | QAPGKGLEWVSTISSGGSY | |
| | | VKGRFTISRDNSKNTLYLQMN | | TYYPDSVKGRFTISRDNSK | |
| | | SLRAEDTAVYYCARHPDYDGV | | NTLYLQMNSLRAEDTAVYY | |
| | | WFAYWGQGTLVTVSS | | CARHPDYDGVWFAYWGQGT | |
| | | | | LVTVSS | |
| hHC4- | 152 | EVQLVESGGGLVKPGGSLRLS | 289 | EVQLVESGGGLVKPGGSLR | U.S. Pat. No. |
| K- | | CAASGFTFNSYGMSWVRQAPG | | LSCAASGFTFNSYGMSWVR | 9346886 B2 |
| humanized | | KGLEWVSSISSGGSYIYYADS | | QAPGKGLEWVSSISSGGSY | |
| | | VKGRFTISRDNAKNSLYLQMN | | IYYADSVKGRFTISRDNAK | |
| | | SLRAEDTAVYYCARHPDYDGV | | NSLYLQMNSLRAEDTAVYY | |
| | | WFAYWGQGTLVTVSS | | CARHPDYDGVWFAYWGQGT | |
| | | | | LVTVSS | |
| hHC4- | 153 | EVQLVESGGGLVKPGGSLRLS | 290 | EVQLVESGGGLVKPGGSLR | U.S. Pat. No. |
| K2- | | CAASGFTFNSYGMSWVRQAPG | | LSCAASGFTFNSYGMSWVR | 9346886 B2 |
| humanized | | KGLEWVSSISSGGSYTYYADS | | QAPGKGLEWVSSISSGGSY | |
| | | VKGRFTISRDNAKNSLYLQMN | | TYYADSVKGRFTISRDNAK | |
| | | SLRAEDTAVYYCARHPDYDGV | | NSLYLQMNSLRAEDTAVYY | |
| | | WFAYWGQGTLVTVSS | | CARHPDYDGVWFAYWGQGT | |
| | | | | LVTVSS | |
| hHC4- | 154 | EVQLLESGGGLVQPGGSLRLS | 291 | EVQLLESGGGLVQPGGSLR | U.S. Pat. No. |
| I- | | CAASGFTFNSYAMSWVRQAPG | | LSCAASGFTFNSYAMSWVR | 9346886 B2 |
| humanized | | KGLEWVSAISSGGSYTYYADS | | QAPGKGLEWVSAISSGGSY | |
| | | VKGRFTISRDNSKNTLYLQMN | | TYYADSVKGRFTISRDNSK | |
| | | SLRAEDTAVYYCAKHPDYDGV | | NTLYLQMNSLRAEDTAVYY | |
| | | WFAYWGQGTLVTVSS | | CAKHPDYDGVWFAYWGQGT | |
| | | | | LVTVSS | |
| hHC4- | 155 | EVQLLESGGGLVQPGGSLRLS | 292 | EVQLLESGGGLVQPGGSLR | U.S. Pat. No. |
| 07- | | CAASGFTFNSYAMSWVRQAPG | | LSCAASGFTFNSYAMSWVR | 9346886 B2 |
| humanized | | KGLEWVSTISSGGSYTYYPDS | | QAPGKGLEWVSTISSGGSY | |
| | | VKGRFTISRDNSKNTLYLQMN | | TYYPDSVKGRFTISRDNSK | |
| | | SLRAEDTAVYYCAKHPDYDGV | | NTLYLQMNSLRAEDTAVYY | |
| | | WFAYWGQGTLVTVSS | | CAKHPDYDGVWFAYWGQGT | |
| | | | | LVTVSS | |
| hHC4- | 156 | EVQLLESGGGLVQPGGSLRLS | 293 | EVQLLESGGGLVQPGGSLR | U.S. Pat. No. |
| 03- | | CAASGFTFNSYGMSWVRQAPG | | LSCAASGFTFNSYGMSWVR | 9346886 B2 |
| humanized | | KGLEWVSAISSGGSYTYYPDS | | QAPGKGLEWVSAISSGGSY | |
| | | VKGRFTISRDNSKNTLYLQMN | | TYYPDSVKGRFTISRDNSK | |
| | | SLRAEDTAVYYCAKHPDYDGV | | NTLYLQMNSLRAEDTAVYY | |
| | | WFAYWGQGTLVTVSS | | CAKHPDYDGVWFAYWGQGT | |
| | | | | LVTVSS | |
| hHC4- | 157 | EVQLVESGGGLVKPGGSLRLS | 294 | EVQLVESGGGLVKPGGSLR | U.S. Pat. No. |
| b- | | CAASGFTFNSYSMNWVRQAPG | | LSCAASGFTFNSYSMNWVR | 9346886 B2 |
| humanized | | KGLEWVSSISSGGSYIYYADS | | QAPGKGLEWVSSISSGGSY | |
| | | VKGRFTISRDNAKNSLYLQMN | | IYYADSVKGRFTISRDNAK | |
| | | SLRAEDTAVYYCARHPDYDGV | | NSLYLQMNSLRAEDTAVYY | |
| | | WFAYWGQGTLVTVSS | | CARHPDYDGVWFAYWGQGT | |
| | | | | LVTVSS | |
| hHC4- | 158 | QVTLKESGPGILQPSQTLSLT | 295 | DVLMTQTPLSLPVSLGDQA | JP2007112734 |
| M- | | CSFSGFSLSTSGMGVSWIRQP | | SISCRSSQSIVHSDGNTYL | |
| humanized | | SGKGLEWLAHIYWDDDKRYNP | | EWYLQKPGQSPKLLIYKVS | |
| | | SLKSRLTISKDTSRNQVFLKI | | NRFSGVPDRFSGSGSGTDF | |
| | | TSVDTADTATYYCARSSSYYD | | TLKISRVEAEDLGVYYCFQ | |
| | | CFAYWGQGTLVTVSA | | GSHVPYTFGGGTKLEIK | |

Several human, humanized, and chimeric anti-CDCP1 antibodies are disclosed in the patent literature (see, e.g., Table A above depicting the heavy chain, heavy chain variable region, light chain, and/or light chain variable regions of such antibodies. Such sequences can be found, for example, in Japanese Application Publication No. JP 2007/112734, PCT Application Publication No. WO 2022/212876, as well as U.S. Application Publication Nos. 2023/0050380 A1, 2022/0389113 A1, 2022/011954 A1, 2008/0008719 A1, and U.S. Pat. No. 9,346,886, which are herein incorporated by reference.

In some embodiments, the targeting agent, antibody, or binding fragment thereof, comprises a heavy chain (or heavy chain variable region) comprising an amino acid sequence at least about 90%, at least about 91%, at least about 92%, at least about 93%, at least about 94%, at least about 95%, at least about 96%, at least about 97%, at least about 98%, at least about 99%, or about 100% identical to the amino acid sequence of any one of SEQ ID NOS: 23-158 and a light chain (or a light chain variable region) comprising an amino acid sequence at least about 90%, at least about 91%, at least about 92%, at least about 93%, at least about 94%, at least about 95%, at least about 96%, at least about 97%, at least about 98%, at least about 99%, or about 100% identical to the amino acid sequence of SEQ ID NOs: 159-295.

In some embodiments, the targeting agent, antibody, or binding fragment thereof, comprises a heavy chain comprising an amino acid sequence at least about 90%, at least about 91%, at least about 92%, at least about 93%, at least about 94%, at least about 95%, at least about 96%, at least about 97%, at least about 98%, at least about 99%, or about 100% identical to the amino acid sequence of SEQ ID NO: 1.

In some embodiments, the targeting agent, antibody, or binding fragment thereof, comprises a light chain comprising an amino acid sequence at least about 90%, at least about 91%, at least about 92%, at least about 93%, at least about 94%, at least about 95%, at least about 96%, at least about 97%, at least about 98%, at least about 99%, or about 100% identical to the amino acid sequence of SEQ ID NO: 5.

In some embodiments, the targeting agent, antibody, or binding fragment thereof, comprises a heavy chain comprising an amino acid sequence at least about 90%, at least about 91%, at least about 92%, at least about 93%, at least about 94%, at least about 95%, at least about 96%, at least about 97%, at least about 98%, at least about 99%, or about 100% identical to the amino acid sequence of SEQ ID NO: 10.

In some embodiments, the targeting agent, antibody, or binding fragment thereof, comprises a light chain comprising an amino acid sequence at least about 90%, at least about 91%, at least about 92%, at least about 93%, at least about 94%, at least about 95%, at least about 96%, at least about 97%, at least about 98%, at least about 99%, or about 100% identical to the amino acid sequence of SEQ ID NO: 11.

In some embodiments, the targeting agent, antibody, or binding fragment thereof comprises:
(i) a heavy chain variable region (VH) that comprises:
   (a) a CDRH1 comprising and/or consisting of the amino acid sequence of SEQ ID NO: 2,
   (b) a CDRH2 comprising and/or consisting of the amino acid sequence of SEQ ID NO: 3, and
   (c) a CDRH3 comprising and/or consisting of the amino acid sequence of SEQ ID NO: 4,
and (ii) a light chain variable region (VL) that comprises:
   (a) a CDRL1 comprising and/or consisting of the amino acid sequence of SEQ ID NO: 6,
   (b) a CDRL2 comprising and/or consisting of the amino acid sequence of SEQ ID NO: 7, and
   (c) a CDRL3 comprising and/or consisting of the amino acid sequence of SEQ ID NO: 8.

In some embodiments, the targeting agent, antibody, or binding fragment thereof comprises:
(i) a heavy chain variable region (VH) that comprises:
   (a) a CDRH1 comprising and/or consisting of the amino acid sequence of SEQ ID NO: 12,
   (b) a CDRH2 comprising and/or consisting of the amino acid sequence of SEQ ID NO: 13, and
   (c) a CDRH3 comprising and/or consisting of the amino acid sequence of SEQ ID NO: 14,
and (ii) a light chain variable region (VL) that comprises:
   (a) a CDRL1 comprising and/or consisting of the amino acid sequence of SEQ ID NO: 6,
   (b) a CDRL2 comprising and/or consisting of the amino acid sequence of SEQ ID NO: 7, and
   (c) a CDRL3 comprising and/or consisting of the amino acid sequence of SEQ ID NO: 8.

In some embodiments, the targeting agent, antibody, or binding fragment thereof, comprises a heavy chain comprising an amino acid sequence at least about 90%, at least about 91%, at least about 92%, at least about 93%, at least about 94%, at least about 95%, at least about 96%, at least about 97%, at least about 98%, at least about 99%, or about 100% identical to the amino acid sequence of SEQ ID NOs: 16, 17, and/or 18.

In some embodiments, the targeting agent, antibody, or binding fragment thereof, comprises a heavy chain comprising an amino acid sequence at least about 90%, at least about 91%, at least about 92%, at least about 93%, at least about 94%, at least about 95%, at least about 96%, at least about 97%, at least about 98%, at least about 99%, or about 100% identical to the amino acid sequence of SEQ ID NO: 15.

In some embodiments, the targeting agent, antibody, or binding fragment thereof, comprises a light chain comprising an amino acid sequence at least about 90%, at least about 91%, at least about 92%, at least about 93%, at least about 94%, at least about 95%, at least about 96%, at least about 97%, at least about 98%, at least about 99%, or about 100% identical to the amino acid sequence of SEQ ID NOs: 20, 21, and/or 22.

In some embodiments, the targeting agent, antibody, or binding fragment thereof, comprises a light chain comprising an amino acid sequence at least about 90%, at least about 91%, at least about 92%, at least about 93%, at least about 94%, at least about 95%, at least about 96%, at least about 97%, at least about 98%, at least about 99%, or about 100% identical to the amino acid sequence of SEQ ID NO: 19.

In some embodiments, the targeting agent, antibody, or binding fragment thereof comprises:
(i) a heavy chain variable region (VH) that comprises:
   (a) a CDRH1 comprising and/or consisting of the amino acid sequence of SEQ ID NO: 16,
   (b) a CDRH2 comprising and/or consisting of the amino acid sequence of SEQ ID NO: 17, and
   (c) a CDRH3 comprising and/or consisting of the amino acid sequence of SEQ ID NO: 18,
and (ii) a light chain variable region (VL) that comprises:
   (a) a CDRL1 comprising and/or consisting of the amino acid sequence of SEQ ID NO: 20,
   (b) a CDRL2 comprising and/or consisting of the amino acid sequence of SEQ ID NO: 21, and
   (c) a CDRL3 comprising and/or consisting of the amino acid sequence of SEQ ID NO: 22.

In certain embodiments, the targeting agent, antibody, or binding fragment thereof, described herein comprises an Fc domain. The Fc domain can be derived from IgA (e.g., IgA1 or IgA2), IgG, IgE, or IgG (e.g., IgG1, IgG2, IgG3, or IgG4). In some embodiments, the Fc domain comprises wild type sequence of an Fc domain. In some embodiments, the Fc domain comprises one or more mutations resulting in altered biological activity. For example, mutations may be introduced into the Fc domain to increase the homogeneity during the production of the recombinant protein. In some embodiments, the Fc domain is the Fc domain of human IgG. In some embodiments, the lysine located in the C-terminal position of the Fc domain is deleted to increase the homogeneity during the production of the recombinant protein. In some embodiments, the lysine located in the C-terminal position of the Fc domain is present.

In certain embodiments, the polypeptide comprising the targeting agent, antibody, or binding fragment thereof, described herein is encoded by a cDNA polynucleotide sequence. As is well-understood in the art, introduction of the cDNA into a competent mammalian cell will result in the production of the polypeptide comprising the targeting agent, antibody, or binding fragment thereof. Exemplary methods of antibody production by these means are disclosed in at least U.S. Pat. Nos. 8,008,449, 10,934,571 and 11,339,215, which are herein incorporated by reference.

In one embodiment, the cDNA comprises a polynucleotide encoding a polypeptide comprising an immunoglobulin heavy chain or a fragment thereof comprising a VH comprising CDRs H1, H2, and H3 with the amino acid sequences set forth in SEQ ID NOs: 2, 3, and 4, respectively.

In one embodiment, the cDNA comprises a polynucleotide encoding a polypeptide comprising an immunoglobulin heavy chain or a fragment thereof comprising a VH with the amino acid sequence set forth in SEQ ID NO: 1.

In one embodiment, the cDNA comprises a polynucleotide encoding a polypeptide comprising an immunoglobulin light chain or a fragment thereof comprising a VL comprising CDRs L1, L2, and L3 with the amino acid sequences set forth in SEQ ID NOs: 6, 7, and 8, respectively.

In one embodiment, the cDNA comprises a polynucleotide encoding a polypeptide comprising an immunoglobulin heavy chain or a fragment thereof comprising a VL with the amino acid sequence set forth in SEQ ID NO: 5.

Also provided by the present disclosure is a targeting agent, antibody, or binding fragment thereof, that binds to the same epitope (e.g. CDCP1) as any of the antibodies, or binding fragment thereofs thereof, described herein. For example, antibody competition assay (and overlapping epitope analysis) can be assessed by surface plasmon resonance (SPR) or bio-layer interferometry (BLI), as described in detail herein.

The antibodies and binding fragment thereofs provided by the invention include monoclonal antibodies, polyclonal antibodies, antibody fragments (e.g., Fab, Fab′, F(ab′)₂, Fv, Fc, etc.), chimeric antibodies, bispecific antibodies, heteroconjugate antibodies (e.g. antibody-drug conjugates), single chain (ScFv), mutants thereof, fusion proteins comprising an antibody portion, domain antibodies (dAbs), humanized antibodies, and any other modified configuration of the immunoglobulin molecule that comprises an antigen recognition site of the required specificity, including glycosylation variants of antibodies, amino acid sequence variants of antibodies, and covalently modified antibodies. The antibodies and binding fragment thereofs may be murine, rat, human, or any other origin (including chimeric or humanized antibodies). In some embodiments, the antibody is a monoclonal antibody. In some embodiments, the antibody is a chimeric, humanized or human antibody. In certain embodiments, the antibody is an antibody-drug conjugate.

In some embodiments, the anti-CDCP1 antibody or antibody fragment thereof comprises one or more conservative amino acid substitutions. A person of skill in the art will recognize that a conservative amino acid substitution is a substitution of one amino acid with another amino acid that has similar structural or chemical properties, such as, for example, a similar side chain. Exemplary conservative substitutions are described in the art, for example, in Watson et al., Molecular Biology of the Gene, The Benjamin/Cummings Publication Company, 4th Ed. (1987).

“Conservative modifications” refer to amino acid modifications that do not significantly affect or alter the binding characteristics of the antibody containing the amino acid sequences. Conservative modifications include amino acid substitutions, additions and deletions. Conservative substitutions are those in which the amino acid is replaced with an amino acid residue having a similar side chain. The families of amino acid residues having similar side chains are well defined and include amino acids with acidic side chains (e.g., aspartic acid, glutamic acid), basic side chains (e.g., lysine, arginine, histidine), nonpolar side chains (e.g., alanine, valine, leucine, isoleucine, proline, phenylalanine, methionine), uncharged polar side chains (e.g., glycine, asparagine, glutamine, cysteine, serine, threonine, tyrosine, tryptophan), aromatic side chains (e.g., phenylalanine, tryptophan, histidine, tyrosine), aliphatic side chains (e.g., glycine, alanine, valine, leucine, isoleucine, serine, threonine), amide (e.g., asparagine, glutamine), beta-branched side chains (e.g., threonine, valine, isoleucine) and sulfur-containing side chains (cysteine, methionine). Furthermore, any native residue in the polypeptide may also be substituted with alanine, as has been previously described for alanine scanning mutagenesis (MacLennan et al. (1998) Acta Physiol Scand Suppl 643:55-67; Sasaki et al. (1998) Adv Biophys 35:1-24). Amino acid substitutions to the antibodies of the invention may be made by known methods for example by PCR mutagenesis (U.S. Pat. No. 4,683,195).

In some embodiments, the antibody or fragment thereof comprises a variable heavy chain sequence that comprises an amino acid sequence with at least about 95%, about 96%, about 97%, about 98%, or about 99%, sequence identity to the amino acid sequence set forth in SEQ ID NOs: 1; 15; 23-158. In other embodiments, the antibody or fragment thereof retains the binding and/or functional activity of an antibody or fragment thereof that comprises the variable heavy chain sequence of SEQ ID NOs: 1; 15; 23-158. In still further embodiments, the antibody or fragment thereof comprises the variable heavy chain sequence of SEQ ID NOs: 1; 15; 23-158 and have one or more conservative amino acid substitutions, e.g., 1, 2, 3, 4, 5, 1-2, 1-3, 1-4 or 1-5 conservative amino acid substitutions in the heavy chain variable sequence.

In particular embodiments, the antibody or fragment thereof comprises a variable heavy chain sequence with at least about 95%, about 96%, about 97%, about 98%, or about 99% sequence identity to the antibody or fragment thereof heavy chain variable region sequence set forth in SEQ ID NOs: 1; 15; 23-158, comprises one or more conservative amino acid substitutions in a framework region (based on the numbering system of Kabat), and retains the binding and/or functional activity of an antibody or fragment thereof that comprises a variable heavy chain sequence as set forth in SEQ ID NOs: 1; 15; 23-158 and a variable light chain sequence as set forth in SEQ ID NOs: 5; 19; 159-295.

In some embodiments, the antibody or fragment thereof comprises a variable light chain sequence that comprises an amino acid sequence with at least about 95%, about 96%, about 97%, about 98%, or about 99%, sequence identity to the amino acid sequence set forth in SEQ ID NOs: 5; 19; 159-295. In other embodiments, the antibody or fragment thereof retains the binding and/or functional activity of an antibody or fragment thereof that comprises the variable light chain sequence of SEQ ID NOs: 5; 19; 159-295. In still further embodiments, the antibody or fragment thereof comprises the variable light chain sequence of SEQ ID Nos: 5; 19; 159-295 and have one or more conservative amino acid substitutions, e.g., 1, 2, 3, 4, 5, 1-2, 1-3, 1-4 or 1-5 conservative amino acid substitutions in the light chain variable sequence.

In particular embodiments, the antibody or fragment thereof comprises a variable light chain sequence with at least about 95%, about 96%, about 97%, about 98%, or about 99% sequence identity to the antibody or fragment thereof light chain variable region sequence set forth in SEQ ID NOs: 5; 19; 159-295, comprises one or more conservative amino acid substitutions in a framework region (based on the numbering system of Kabat), and retains the binding and/or functional activity of an antibody or fragment thereof that comprises a variable heavy chain sequence as set forth in SEQ ID NOs: 1; 15; 23-158 and a variable light chain sequence as set forth in SEQ ID NOs: 5; 19; 159-295.

The binding affinity of a targeting agent or antibody can be expressed as an equilibrium dissociation constant (K_{D}) value, which refers to the dissociation rate of a particular antigen-antibody interaction. K_{D} is the ratio of the rate of dissociation, also called the “off-rate (k_{off})”, to the association rate, or “on-rate (kₒₙ)”. Thus, K_{D} equals k_{off}/kₒₙ (dissociation/association) and is expressed as a molar concentration (M), and the smaller the K_{D}, the stronger the affinity of binding. K_{D} values for antibodies can be determined using methods well established in the art. Unless otherwise specified, “binding affinity” refers to monovalent interactions (intrinsic activity; e.g., binding of an antibody to an antigen through a monovalent interaction).

In certain embodiments, the targeting agent, antibody, or binding fragment thereof, of the invention has an affinity (K_{D}) value of or less than about 350 nM, about 325 nM, about 323.10 nM, about 300 nM, about 286.44 nM, about 275 nM, about 250 nM, about 232.13 nM, about 225 nM, about 219.13 nM, about 200 nM, about 195.54 nM, about 175 nM, about 158 nM, about 150 nM, about 125 nM, or about 100 nM.

In some embodiments, the targeting agent, antibody, or binding fragment thereof, binds an epitope (e.g. CDCP1) with a K_{D} value of or less than about 95 nM, about 90 nM, about 80 nM, about 79.89 nM, about 75 nM, about 70 nM, about 69.50 nM, about 65 nM, about 63.44 nM, about 60 nM, about 55 nM, about 52.88 nM, about 50 nM, about 45 nM, about 44.50 nM, about 41.99 nM, about 40 nM, about 35 nM, about 30 nM, about 25 nM, about 20 nM, about 10 nM, about 5 nM, or about 1 nM.

In some embodiments, the targeting agent, antibody, or binding fragment thereof, binds an epitope (e.g. CDCP1) with a K_{D} value of or less than about 5 nM, about 4.5 nM, about 4 nM, about 3.5 nM, about 3.12 nM, about 3 nM, about 2.90 nM, about 2.5 nM, about 2 nM, about 1.5 nM, about 1 nM, about 900 pM, about 800 pM, about 700 pM, about 600 pM, about 500 pM, about 400 pM, about 300 pM, about 250 pM, about 200 pM, about 150 pM, about 100 pM, about 50 pM, about 40 pM, about 30 pM, about 25 pM, about 20 pM, about 15 pM, about 10 pM, about 5 pM, or about 1 pM.

The value of K_{D} can be determined directly by well-known methods, and can be computed even for complex mixtures by methods such as those, for example, set forth in Caceci et al., (1984, Byte 9:340-362). For example, the K_{D} may be established using a double-filter nitrocellulose filter binding assay such as that disclosed by Wong & Lohman (1993, Proc. Natl. Acad. Sci. USA 90:5428-5432). Other standard assays to evaluate the binding ability of ligands such as antibodies towards target antigens are known in the art, including for example, ELISAs, Western blots, RIAs, and flow cytometry analysis, and other assays exemplified elsewhere herein.

One exemplary method for measuring binding affinity (K_{D}) value is surface plasmon resonance (SPR), typically using a biosensor system such as a BIACORE® system. SPR refers to an optical phenomenon that allows for the analysis of real-time biospecific interactions by detection of alterations in protein concentrations within a biosensor matrix, for example using the BIACORE® system. BIAcore kinetic analysis comprises analyzing the binding and dissociation of an antigen from a chip with an immobilized molecule (e.g., a molecule comprising an antigen-binding domain), on their surface; or the dissociation of an antibody, or binding fragment thereof, from a chip with an immobilized antigen.

In certain embodiments, the SPR measurement is conducted using a BIACORE® T100 or T200 instrument. For example, a standard assay condition for surface plasmon resonance can be based on antibody immobilization of approximately 100-500 Response Units (RU) of IgG on the SPR chip. Purified target proteins are diluted in buffer to a range of final concentrations and injected at a requisite flow rate (e.g., 10-100 μl/min) to allow the calculation of Ka. Dissociation is allowed to proceed to establish off-rate, followed by 3 M MgCl₂ (or 20 mM NaOH) for regeneration of the chip surface. Sensorgrams are then analyzed using a kinetics evaluation software package. In an exemplary embodiment, the SPR assay is according to the conditions as set forth in the Examples.

In certain embodiments, the binding affinity (K_{D}) value is measured using solution-based kinetic exclusion assay (KinExA™). In a particular embodiment, the KinExA measurement is conducted using a KinExA™ 3200 instrument (Sapidyne). The Kinetic Exclusion Assay (KinExA™) is a general purpose immunoassay platform (basically a flow spectrofluorimeter) that is capable of measuring equilibrium dissociation constants, and association and dissociation rate constants for antigen/antibody interactions. Since KinExA™ is performed after equilibrium has been obtained it is an advantageous technique to use for measuring the K_{D} of high affinity interactions where the off-rate of the interaction may be very slow. The KinExA™ methodology can be conducted generally as described in Drake et al., (2004) Analytical Biochem. 328, 35-43.

Another method for determining the K_{D} of an antibody is by using Bio-Layer Interferometry (BLI), typically using OCTET® technology (e.g., Octet QKe system) from ForteBio. In certain embodiments, the BLI measurement is conducted according to the following: sensor tips coated with a proprietary anti-human antibody (ForteBio) undergo BLI signal stabilization by dipping in running buffer (such as 10 mM Hepes Buffered Saline (HBS) containing 0.05% tween-20) for 120s. The antibody is then captured by dipping the sensors into a running buffer solution (buffer may contain 1-10 ug/mL of the antibody) for 300s. The signal is then stabilized by dipping the sensor tips back into running buffer for 120s. The tips are then transferred into solution containing the cognate antigen. The binding of antibody-antigen is measured over 180s prior to the sensor tips being transferred to running buffer in order to monitor receptor dissociation over 180s.

In the case of CDCP1, typically a 7-point dose response of the antigen (may range from 1-2 nM in doubling dilutions) is measured. Additionally, sensor tips with no antibody captured are exposed to the antigen in order to monitor non-specific binding of the receptors to the sensor tips. A 2^{nd} reference type also includes a tip with antibody captured upon on it but with subsequent exposure to running buffer only with no antigen. This allows for double-referencing to eliminate both non-specific binding as well as system noise and the underlying baseline drift attributed to the antibody dissociating from the anti-human Fc sensor tip. The raw under goes double reference subtraction and is then fit to a 1:1 Langmuir type binding model to determine affinity and kinetic parameters.

In some embodiments, the CDCP1 is a human CDCP1, cyno CDCP1 or mouse CDCP1. In general, an anti-CDCP1 antibody should bind to CDCP1 with high affinity. It is desirable that the anti-CDCP1 antibody have binding affinities (K_{D}) to human CDCP1 in low nanomolar range, such as about 40 nM or lower. In some embodiments, the CDCP1 is a human CDCP1 and the K_{D} value is about 40 nM, about 45 nM or about 50 nM. In some embodiments, the CDCP1 is a cyno CDCP1 and the K_{D} value is about 62 nM, about 64 nM, about 66 nm, about 68 nM, or about 70 nM.

### Drug Moieties

In some embodiments, drug moiety is a cytotoxic agent, an immunomodulating agent, an imaging agent, a chemotherapeutic agent, or a therapeutic protein.

In some embodiments, the drug moiety is a small molecule having a molecular weight preferably <about 5 kDa, more preferably <about 4 kDa, more preferably <about 3 kDa, most preferably <about 1.5 kDa or <about 1 kDa.

In some embodiments, the drug moiety has an IC₅₀ of about less than about 1 nM.

In some embodiments, the drug moiety has an IC₅₀ of about greater than 1 nM, for example, the therapeutic agent has an IC₅₀ of about 1 to about 50 nM.

Some drug moieties having an IC₅₀ of greater than about 1 nM (e.g., “less potent drugs”) are unsuitable for conjugation with an antibody using art-recognized conjugation techniques. Without wishing to be bound by theory, such drug moieties have a potency that is insufficient for use in targeted antibody-drug conjugates using conventional techniques as sufficient copies of the drug (i.e., more than 8) cannot be conjugated using art-recognized techniques without resulting in diminished pharmacokinetic and physiochemical properties of the conjugate. However sufficiently high loadings of these less potent drugs can be achieved using the conjugation strategies described herein thereby resulting in high loadings of the therapeutic agent while maintaining the desirable pharmacokinetic and physiochemical properties. Thus, in some embodiments, the disclosure also relates to an antibody-drug conjugate which includes an antibody, a linker, and at least eight drug moieties moieties, wherein the therapeutic agent has an IC₅₀ of greater than about 1 nM.

In some embodiments, the small molecule therapeutic agents used in this disclosure (e.g., antiproliferative (cytotoxic and cytostatic) agents capable of being linked to a targeting moiety via the linker(s) of the disclosure) include cytotoxic compounds (e.g., broad spectrum), angiogenesis inhibitors, ceil cycle progression inhibitors, PI3K/m-TOR/AKT pathway inhibitors, MAPK signaling pathway inhibitors, kinase inhibitors, protein chaperones inhibitors, HDAC inhibitors, PARP inhibitors, nicotinamide phosphoribosyl transferase (NAMPT) inhibitors, Wnt Hedgehog signaling pathway inhibitors and RNA polymerase inhibitors.

Broad spectrum cytotoxins include, but are not limited to, DNA-binding, intercalating or alkylating drugs, microtubule stabilizing and destabilizing agents, platinum compounds, topoisomerase inhibitors (including topoisomerase I and topoisomerase II inhibitors) and protein synthesis inhibitors.

In some embodiments, the drug moiety comprises one or more cGAS/stimulator of interferon genes (STING) pathway agonists. Non-limiting examples of STING agonists include DMXAA, ADUS100/MIW815, MK-1454, MK-2118, SB11285, GSK3745417, BMS-986301, BI-STING (BI 1387446), E7766, TAK-676, SNX281, SYNB1891. Additional non-limiting examples of STING agonists and combinations with other cytotoxic agents and/or ENPP1 inhibitors can be found in Amouzegar et al., Cancers 13:2695 (2021), which is incorporated herein by reference in its entirety.

Exemplary DNA-binding, intercalation or alkylating drugs include, but are not limited to, CC-1065 and its analogs, anthracyclines (doxorubicin, epirubicin, idarubicin, daunorubicin, nemorubicin and its derivatives, PNU-159682), bisnapththalimide compounds such as elinafide (LU79553).and its analogs, alkylating agents, such as calicheamicins, dactinomycins, mitomycins, pyrrolobenzodiazepines, indolinobenzodiazepines and the like. Exemplary CC-1065 analogs include, but are not limited to, duocarmycin SA, duocarmycin A, duocarmycin CI, duocarmycin C2, duocarmycin B 1, duocarmycin B2, duocarmvcin D, DU-86, KW-2189. adozeiesin, bizelesin, carzeiesin. seco-adozelesin, and related analogs and prodrug forms, examples of which are described in U.S. Pat. Nos. 5,475,092; 5,595,499; 5,846,545; 6,534,660; 6,586,618; 6,756,397 and 7,049,316. Doxorubicin and its analogs include those described in U.S. Pat. No. 6,630,579. Calicheamicins include, e.g., enediynes, e.g., esperamicin, and those described in U.S. Pat. Nos. 5,714,586 and 5,739,116. Duocarmycins include those described in U.S. Pat. Nos. 5,070,092; 5,101,038; 5, 187,186; 6,548,530; 6,660,742; and 7,553,816 B2; and Li et al., Tel Letts., 50:2932-2935 (2009), the disclosures of all of which are incorporated by reference herein in their entireties.

Exemplary topoisomerase inhibitors (e.g. topoisomerase I and/or topoisomerase II) include, but are not limited to, camptothecin, camptothecin derivatives, camptothecin analogs and non-natural camptothecins, such as, for example, exatecan, Dxd, Sn-38 (7-ethyl-10-hydroxy-camptothecin), CPT-11 (irinotecan), GI-147211C, topotecan, 9-aminocamptothecin, 7-hydroxymethyl camptothecin, 7-aminomethyl camptothecin, 10-hydroxy camptothecin, (20S)-camptothecin, rubitecan, gimatecan, karenitecin, silatecan, lurtotecan, diflomotecan, belotecan, lurtotecan and S39625, and any analogues thereof. Non-limiting examples of other topoisomerase inhibitors (e.g. topoisomerase I and/or topoisomerase II) that can be used in the present disclosure include those described in WO 2020/00880 and WO 2021/148501, the disclosures of each of which are incorporated by reference herein in their entireties. Non-limiting examples of other camptothecin compounds that can be used in the present disclosure include those described in J. Med. Chem., 29:2358-2363 (1986); J. Med. Chem., 23:554 (1980); J. Med. Chem., 30:1774 (1987), the disclosures of each of which are incorporated by reference herein in their entireties. In some embodiments, the drug moiety is exatecan and/or an analogue thereof. In some embodiments, the drug moiety is Dxd and/or an analogue thereof.

Non-limiting examples of pyrrolobenzodiazepines (PBD) and analogs thereof include, but are not limited to, those described in Denny, Exp. Opin. Ther. Patents., 10(4): 459-474 (2000), Antonow and Thurston, Chem Rev., 2815-2864 (2010), Min et al., ACS Omega 5:25798-25809 (2020), and Hartley Exp. Opin. Biol. Therapy 7:931-943 (2020), the disclosures of each of which is incorporated by reference herein in their entireties.

Exemplary microtubule stabilizing and destabilizing agents include, but are not limited to, taxane compounds, such as paclitaxel, docetaxel, tesetaxel and carbazitaxel; maytansinoids, auristatins and analogs thereof, vinca alkaloid derivatives, epothilones and cryptophycins.

Exemplary maytansinoids or maytansinoid analogs include, but are not limited to maytansinoi and maytansinol analogs, maytansine or DM-i and DM-4 are those described in U.S. Pat. Nos. 5,208,020; 5,416,064; 6,333,410, 6,441,163; 6,716,821; RE39,151 and 7,276,497. In certain embodiments, the cytotoxic agent is a maytansinoid, another group of anti-tubulin agents (ImmunoGen, Inc.; see also Chari et al., 1992, Cancer Res. 52:127-131), maytansinoids or maytansinoid analogs. Examples of suitable maytansinoids include, but are not limited to, maytansinol and maytansinol analogs. Non-limiting examples of suitable maytansinoids are disclosed in U.S. Pat. Nos. 4,424,219; 4,256,746; 4,294,757; 4,307,016; 4,313,946; 4,315,929; 4,331,598; 4,361,650; 4,362,663; 4,364,866, 4,450,254; 4,322,348; 4,371,533, 6,333,410; 5,475,092; 5,585,499; and 5,846,545, which are incorporated by reference herein in their entireties.

Exemplary auristatins include, but are not limited to, auristatin E (also known as a derivative of dolastatin-10), auristatin EB (AEB), auristatin EFP (AEFP), monomethyl auristatin E (MMAE), monomethyl auristatin F (MMAF), auristatin F, auristatin F phenylenediamine (AFP), auristatin F hydroxylpropylamide (AF FTP A), monomethyl auristatin F hydroxylpropylamide (MMAF HP A), and dolastatin. Non-limiting examples of suitable auristatins are also described in U.S. Publication Nos. 2003/0083263, 2011/0020343, and 2011/0070248; PCT Application Publication Nos. WO 09/117531, WO 2005/081711, WO 04/010957; WO 02/088172 and WO 01/24763, and U.S. Pat. Nos. 7,498,298; 6,884,869; 6,323,315; 6,239,104; 6,124,431; 6,034,065; 5,780,588; 5,767,237, 5,665,860; 5,663,149; 5,635,483, 5,599,902; 5,554,725; 5,530,097; 5,521,284; 5,504,191; 5,410,024; 5,138,036; 5,076,973; 4,986,988; 4,978,744; 4,879,278; 4,816,444; and 4,486,414, the disclosures of each of which are incorporated herein by reference in their entirety. In some embodiments, the drug moiety is monomethyl auristatin E (MMAE) and/or an analogue thereof.

Exemplary vinca alkaloids include, but are not limited to, vincristine, vinblastine, vindesine, and navelbine (vinorelbine). Suitable Vinca alkaloids that can be used in the present disclosure are also disclosed in U.S. Publication Nos. 2002/0103136 and 2010/0305149, and in U.S. Pat. No. 7,303,749 B1, the disclosures of each of which are incorporated herein by reference in their entirety.

Exemplary epothilone compounds include, but are not limited to, epothilone A, B, C, D, E and F, and derivatives thereof. Suitable epothilone compounds and derivatives thereof are described, for example, in U.S. Pat. Nos. 6,956,036; 6,989,450, 6,121,029; 6,117,659; 6,096,757, 6,043,372; 5,969,145; and 5,886,026; and WO 97/19086; WO 98/08849; WO 98/22461; WO 98/25929; WO 98/38192, WO 99/01124; WO 99/02514; WO 99/03848; WO 99/07692; WO 99/27890; and WO 99/28324; the disclosures of all of which are incorporated herein by reference in their entireties.

Non-limiting examples of cryptophycin compounds are described in U.S. Pat. Nos. 6,680,311 and 6,747,021, the disclosures of each of which are incorporated herein by reference in their entireties.

Exemplary platinum compounds include, but are not limited to, cisplatin (PLATINOL®), carboplatin (PARAPLATIN®), oxaliplatin (ELOXATINE®), iproplatin, ormaplatin, and tetraplatin,

Non-limiting examples of other classes of compounds or compounds with these or other cytotoxic modes of action may be selected, including, e.g., mitomycin C, mitomycin A, daunorubicin, doxorubicin, morpholino-doxorubicin, cyanomorpholino-doxorubicin, aminopterin, bleomycin, 1-(chloromethyl)-2,3-dihydro-1H-benzo[e]indol-5-ol, pyrridinobenzodiazepines (PDD), pyrrolobenzodiazepine (PBD) and polyamide and dimers thereof. Non-limiting examples of other suitable cytotoxic agents include puromycins, topotecan, rhizoxin, echinomycin, combretastatin, netropsin, estramustine, cryptophysins, cemadotin, discodermolide, eleutherobin, and mitoxantrone.

Angiogenesis inhibitors include, but are not limited to, MetAP2 inhibitors, VEGF inhibitors, PIGF inhibitors, VGFR inhibitors, PDGFR inhibitors, MetAP2 inhibitors. Exemplary VGFR and PDGFR inhibitors include, but are not limited to, sorafenib (Nexavar), sunitinib (Sutent) and vatalanib. Exemplary MetAP2 inhibitors include fumagillol analogs, meaning any compound that includes the fumagillin core structure, including fumagiilamine, that inhibits the ability of MetAP-2 to remove NH2-terminal methionines from proteins as described in Rodeschini et al., J. Org. Chem., 69, 357-373, 2004 and Liu, et al., Science 282, 1324-1327, 1998, Non limiting examples of “fumagillol analogs” are disclosed in J Org. Chem., 69, 357, 2004; J. Org. Chem., 70, 6870, 2005; European Patent Application 0 354 787; J. Med. Chem., 49, 5645, 2006; Bioorg. Med. Chem., 11, 5051, 2003; Bioorg. Med. Chem., 14, 91, 2004; Tel Lett 40, 4797, 1999; WO 99/61432; U.S. Pat. Nos. 6,603,812; 5,789,405; 5,767,293; 6,566,541; and 6,207,704, the disclosures of all of which are incorporated by reference herein in their entireties.

Exemplary ceil cycle progression inhibitors include, but are not limited to, CDK inhibitors such as BMS-387032 and PD0332991; Rho-kinase inhibitors such as GSK429286; checkpoint kinase inhibitors such as AZD7762; aurora kinase inhibitors such as AZD1 152, MLN8054 and MLN8237; PLK inhibitors such as BI 2536, BI6727 (Volasertib), GSK461364, ON-01910 (Estybon); and KSP inhibitors such as SB 743921, SB 715992 (ispinesib), MK-0731, AZD8477, AZ3146 and ARRY-520.

Exemplary PBK/m-TOR/AKT signaling pathway inhibitors include, but are not limited to, phosphoinositide 3-kinase (PI3K) inhibitors, GSK-3 inhibitors, ATM inhibitors, DNA-PK inhibitors and PDK-1 inhibitors.

Non-limiting examples of exemplary PI3 kinase inhibitors are disclosed in U.S. Pat. No. 6,608,053 (the disclosure of which is incorporated by reference herein in its entirety), and include BEZ235, BGT226, BKM120, CAL101, CAL263, demethoxyviridin, GDC-0941, GSK615, IC871 14, LY294002, Pafomid 529, perifosine, PI-103, PF-04691502, PX-866, SAR245408, SAR245409, SF 1 126, Wortmannin, XL 147 and XL765.

Exemplary AKT inhibitors include, but are not limited to, AT7867.

Exemplary MAPK signaling pathway inhibitors include, but are not limited to, MEK, Ras, JNK, B-Raf and p38 MAPK inhibitors,

Non-limiting exemplary MEK inhibitors are disclosed in U.S. Pat. No. 7,517,994 (the disclosure of which is incorporated by reference herein in its entirety), and include GDC-0973, GSK1120212, MSC1936369B, AS703026, R05126766 and R04987655, PD0325901, AZD6244, AZD 8330 and GDC-0973.

Exemplary B-raf inhibitors include, but are not limited to, CDC-0879, PLX-4032, and SB590885.

Exemplary B p38 M APK inhibitors include, but are not limited to, BIRB 796, LY2228820 and SB 202190.

Receptor tyrosine kinases (RTK) are cell surface receptors which are often associated with signaling pathways stimulating uncontrolled proliferation of cancer cells and neoangiogenesis. Many RTKs, which over express or have mutations leading to constitutive activation of the receptor, have been identified, including, but not limited to, VEGFR, EGFR, FGFR, PDGFR, EphR and RET receptor family receptors. Exemplary specific RTK targets include, but not limited to, ErbB2, FLT-3, c-Kit, and c-Met.

Exemplary inhibitors of ErbB2 receptor (EGFR family) include, but are not limited to, AEE788 (NVP-AEE 788), BIBW2992, (Afatinib), Lapatinib, Erlotinib (Tarceva), and Gefitinib (Iressa).

Exemplary RTK inhibitors targeting more than one signaling pathway (multitargeted kinase inhibitors) include, but are not limited to, AP24534 (Ponatinib) that targets FGFR, FLT-3, VEGFR-PDGFR and Bcr-Abl receptors; ABT-869 (Linifanib) that targets FLT-3 and VEGFR-PDGFR receptors: AZD2171 that targets VEGFR-PDGFR, Flt-1 and VEGF receptors; CHR-258 (Dovitinib) that targets VEGFR-PDGFR, FGFR, Flt-3, and c-Kit receptors; Sunitinib (Sutent) that targets VEGFR, PDGFR, KIT, FLT-3 and CSF-IR; Sorafenib (Nexavar) and Vatalanib that target VEGFR, PDGFR as well as intracellular serine/threonine kinases in the Raf/Mek/Erk pathway.

Exemplary protein chaperon inhibitors include, but are not limited to, HSP90 inhibitors.

Exemplary HSP90 inhibitors include, but are not limited to, 17AAG derivatives, BIIB021, BIIB028, S X-5422, NVP-AUY-922 and KW-2478.

Exemplary WD AC inhibitors include, but are not limited to, Belinostat (PXD101), CUDC-101, Droxinostat, ITF2357 (Givinostat, Gavinostat), JNJ=26481585, LAQ824 (NVP-LAQ824, Dacinostat), LBH-589 (Panobinostat), MC I 568, MGCD0103 (Mocetinostat), M S-275 (Entinostat), PCI-24781, Pyroxamide (NSC 696085), SB939, Trichostatin A and Vorinostat (SAHA).

Exemplary PARP inhibitors include, but are not limited to, iniparib (BSI 201), olaparib (AZD-2281), ABT-888 (Veliparib), AG014699, CEP 9722, MK 4827, KU-0059436 (AZD2281), LT-673, 3-aminobenzamide, A-966492, and AZD2461.

Exemplary NAMPT inhibitors include, but are not limited to, FK866 (AP0866) and CHS828, GPP 78, GMX1778 (CHS828), STF-1 18804, STF-31, CB 300919, CB 30865, GNE-617, IS001, TP201565, Nampt-IN-1, P7C3, MPC-9528, CB30865, MPI0479883 and (£)-N-(5-((4-(((2-(1H-Indol-3-yl)ethyl) (isopropyl)amino)methyl)phenyl)arnino) pentyl)-3-(pyridin-3-yl)acrylamide.

Exemplary Wnt/Hedgehog signaling pathway inhibitors include, but are not limited to, vismodegib (RG3616/GDC-0449), cyclopamine (11-deoxojervine) (Hedgehog pathway inhibitors) and XAV-939 (Wnt pathway inhibitor).

Exemplary RNA polymerase inhibitors include, but are not limited to, amatoxins. Exemplary amatoxins include a-amanitins, β-amanitins, γ-amanitins, ε-amanitins, amanuilin, amanullic acid, amaninamide, amanin, and proamanullin.

Exemplary protein synthesis inhibitors include, but are not limited to, trichothecene compounds.

In some embodiments, the drug moiety is a topoisomerase inhibitor (such as, for example, a non-natural camptothecin compound), vinca alkaloid, kinase inhibitor (e.g., PI3 kinase inhibitor (GDC-0941 and PI-103)), MEK inhibitor, KSP inhibitor, RNA polymerase inhibitor, protein synthesis inhibitor, PARP inhibitor, NAMPT inhibitor, docetaxel, paclitaxel, doxorubicin, duocarmycin, auristatin, dolastatin, calicheamicins, topotecan, SN38, camptothecin, exatecan, nemorubicin and its derivatives, PNU-1.59682, CC1065, elinafide, trichothecene, pyrrolobenzodiazepines, maytansinoids, DNA-binding drugs or a platinum compound, and analogs thereof. In some embodiments, the drug is a derivative of Sn-38, camptothecin, topotecan, exatecan, calicheamicin, nemorubicin, PNU-159682, anthracycline, maytansinoid, taxane, tnchothecene, CC1065, elinafide, vindesine, vinblastine, PI-103, AZD 8330, dolastatin, auristatin E, auristatin F, a duocarmycin compound, ispinesib, pyrrolobenzodiazepine, ARRY-520 and stereoisomers, isosteres and analogs thereof.

In some embodiments, the drug moiety D is a topoisomerase inhibitor having a structure of the formula:

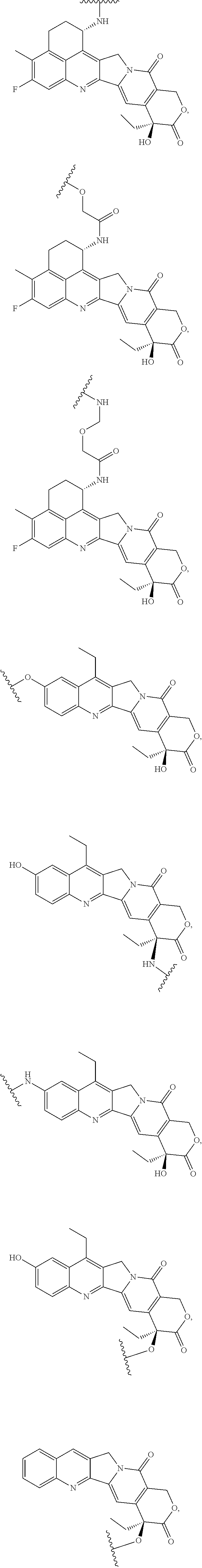

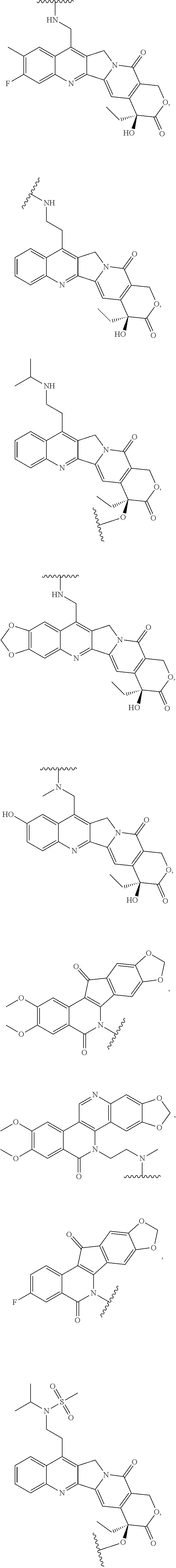

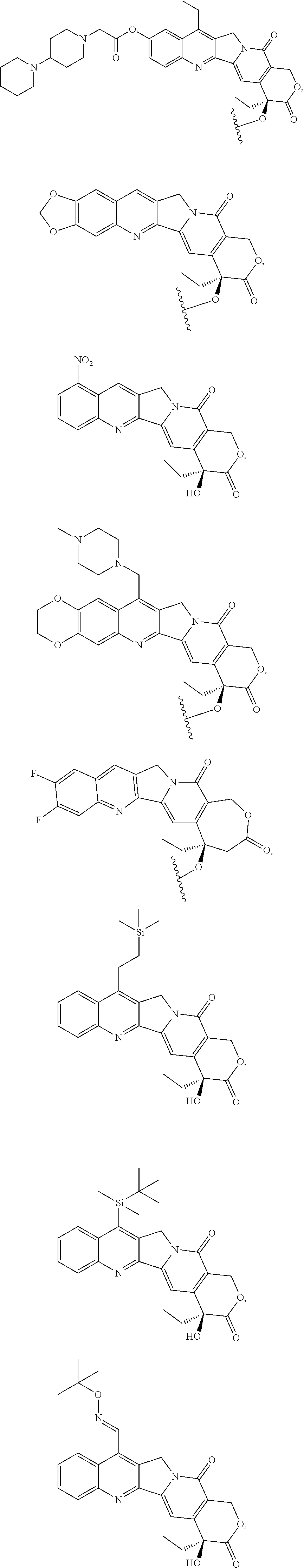

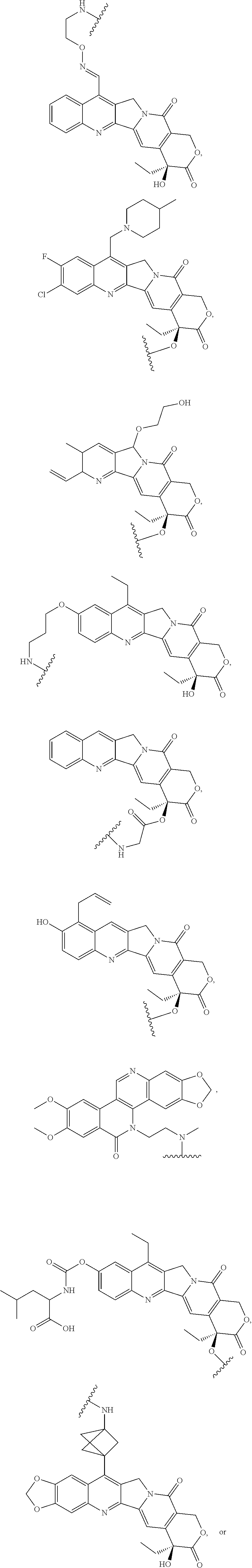

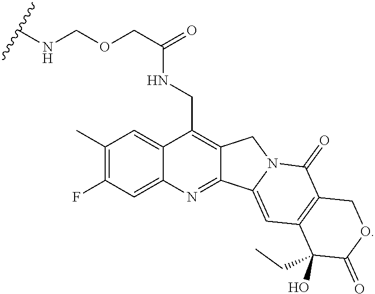 [SMILES: *NCOCC(=O)NCc1c2c(nc3cc(F)c(C)cc13)-c1cc3c(c(=O)n1C2)COC(=O)[C@]3(O)CC]

In some embodiments, the drug moiety used in the disclosure is a combination of two or more drugs, such as, for example, PI3 kinase inhibitors and MEK inhibitors, broad spectrum cytotoxic compounds and platinum compounds; PARI³ inhibitors, NAMPT inhibitors and platinum compounds, broad spectrum cytotoxic compounds and PARP inhibitors.

In some embodiments, the drug moiety used in the disclosure is auristatin F-hydroxypropylamide-L-alanine.

### Linker

In one aspect, a drug moiety, may be linked, either directly or indirectly, to a targeting agent (e.g. an antibody or antibody-binding fragment) to provide a targeted conjugate. In some embodiments, an antibody drug conjugate (ADC) of the disclosure (e.g. an ADC of formula (I)), contains a linker group, wherein the targeting agent (e.g. an antibody or antibody-binding fragment) is attached to the drug moiety through the linker group. In some embodiments, a compound of the disclosure (e.g. formula (III) or salts, solvates, tautomers, isomers or mixtures thereof), contains a linker group, wherein the targeting agent (e.g. an antibody or antibody-binding fragment) is attached to the drug moiety through the linker group. In some embodiments, the linker is a single bond. In a non-limiting example, when the linker is a single bond, the drug moiety is directly attached to a targeting agent (e.g. an antibody or antibody-binding fragment). In some embodiments, a variety of target conjugates are known in the art and can be used with a compound of formula (III) and salts or solvates thereof. In a non-limiting example the target conjugate is an antibody-drug conjugate, wherein one or more compounds of formula (III) are linked to the antibody. In embodiments, the antibody drug conjugates of the present disclosure contain one or multiple compounds of formula (III) or salts, solvates, tautomers, isomers or mixtures thereof.

Any linker suitable for attaching a drug moiety to a targeting agent (e.g. an antibody or antibody-binding fragment) is contemplated by the present disclosure, as would be understood by one of ordinary skill in the art.

In some embodiments, the linker is a bond or is a moiety having 1-200 nonhydrogen atoms selected from C, N, O, S, or halogen, and optionally incorporates alkyl, ether, oxo, carboxyl, carboxamide, carboxamidyl, ester, urethanyl, branched, cyclic, unsaturated, amino acid, heterocyclyl, aryl or heteroaryl moieties. In embodiments, the linker is unbranched or branched, flexible or rigid, short or long and optionally incorporates any combination of moieties as deemed useful. In some embodiments, at least a portion of the linker has a polyalkylene oxide polymeric region. In a non-limiting example, polyalkylene oxide polymeric region are capable of enhancing solubility of the drug moiety. In some embodiments, the linker has a repeating unit of ethylene glycol.

In some embodiments, the linker has a number of repeating ethylene glycol units ranging from about 1 to about 25, or any number therebetween. In some embodiments, the linker includes about 3 to about 20, about 3 to about 5, about 4 to about 15, about 4 to about 8, about 4 to about 6, about 5 to about 12, about 6 to about 10, or about 7 to about 9 ethylene glycol units. In some embodiments, the linker includes about 8 ethylene glycol units.

In some embodiments, at least a portion of the linker includes one or more amino acid moieties. In a non-limiting example, one or more amino acid moieties provides enhanced solubility for the drug moiety and/or provides amino acid sequences to enhance target binding, enhance compatibility with a targeting agent, and/or enhance target binding recognition. In some embodiments, the linker includes one or more amino acid moieties that provide a suitable substrate motif for a protease. In a non-limiting example, when a set of amino acid moieties are incorporated into the linker that provide a substrate motif specific for a selected protease, the drug moiety may be released from a target bound conjugate to provide localized cytotoxic effects.

In some embodiments, the linker includes an alkylene chain. In some embodiments, the alkylene chain is 1, 2, 3, 4, 5, 6, 7, 8, 9, 10, 11 or 12 carbons in length; and suitably the alkylene chain comprises —CH₂— groups. In some embodiments, these substrate motifs are known in the art and are incorporated into the linker as desired to provide selective release from the target bound conjugate. In a non-limiting example, this selectivity is based on known presence of a desired protease within the localized delivery region of the conjugate drug. In some embodiments, other polymeric types of moieties may be incorporated in the linker, including but not limited to polyacids, polysaccharides, or polyamines. In some embodiments, other moieties such as substituted aromatic or heteroaromatic moieties are used to enhance rigidity or provide synthetically accessible sites on substituents therein for linking to reactive moieties or to the drug moiety.

In a non-limiting example, the linker includes ethylene glycol repeating units, and/or an amino acid sequence.

In some embodiments, the linker comprises or consists of the formula:

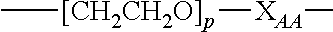

wherein X_{AA} is an amino acid sequence, and p is an integer from 0 to 50. In some embodiments, p is an integer from 1 to 10, 4 to 10, 6 to 10, or 7 to 9. In some embodiments, p is 8.

In some embodiments, the linker comprises or consists of the formula:

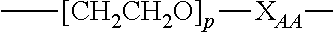

wherein X_{AA} is an amino acid sequence, and p is an integer from 0 to 50. In some embodiments, p is an integer from 1 to 40. In some embodiments, p is an integer from 1 to 30. In some embodiments, p is an integer from 6 to 40. In some embodiments, p is an integer from 8 to 30. In some embodiments, p is an integer from 6 to 20. In some embodiments, p is an integer from 6 to 20. In some embodiments, p is an integer from 8 to 20. In some embodiments, p is an integer from 10 to 30. In some embodiments, p is an integer from 10 to 20. In some embodiments, p is an integer from 1 to 25, 4 to 20, 5 to 15, 6 to 12, or 5 to 10. In some embodiments, p is an integer from 1 to 10, 4 to 10, 6 to 10, or 7 to 9. In some embodiments, p is 8.

In some embodiments, the linker (e.g. L_{A}) comprises or consists of the formula:

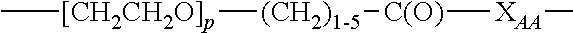

wherein X_{AA} is an amino acid sequence, and p is an integer from 0 to 50. In some embodiments, p is an integer from 1 to 40. In some embodiments, p is an integer from 1 to 30. In some embodiments, p is an integer from 6 to 40. In some embodiments, p is an integer from 8 to 30. In some embodiments, p is an integer from 6 to 20. In some embodiments, p is an integer from 6 to 20. In some embodiments, p is an integer from 8 to 20. In some embodiments, p is an integer from 10 to 30. In some embodiments, p is an integer from 10 to 20. In some embodiments, p is an integer from 1 to 25, 4 to 20, 5 to 15, 6 to 12, or 5 to 10. In some embodiments, p is an integer from 1 to 10, 4 to 10, 6 to 10, or 7 to 9. In some embodiments, p is 8.

In some embodiments, the linker (e.g. L_{A}) comprises or consists of the formula:

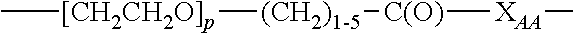

wherein X_{AA} is an amino acid sequence, and p is an integer from 0 to 50, wherein X_{AA} is not Val-Cit or Phe-Lys. In some embodiments, p is an integer from 1 to 40. In some embodiments, p is an integer from 1 to 30. In some embodiments, p is an integer from 6 to 40. In some embodiments, p is an integer from 8 to 30. In some embodiments, p is an integer from 6 to 20. In some embodiments, p is an integer from 6 to 20. In some embodiments, p is an integer from 8 to 20. In some embodiments, p is an integer from 10 to 30. In some embodiments, p is an integer from 10 to 20. In some embodiments, p is an integer from 1 to 25, 4 to 20, 5 to 15, 6 to 12, or 5 to 10. In some embodiments, p is an integer from 1 to 10, 4 to 10, 6 to 10, or 7 to 9. In some embodiments, p is 8. In some embodiments, —(CH₂)₁₋₅— is —(CH₂)₁₋₃—.

In some embodiments, the linker (e.g. L_{A}) comprises or consists of the formula:

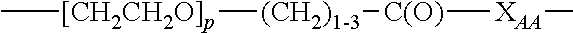

wherein X_{AA} is an amino acid sequence, and p is an integer from 0 to 50. In some embodiments, p is an integer from 1 to 40. In some embodiments, p is an integer from 1 to 30. In some embodiments, p is an integer from 6 to 40. In some embodiments, p is an integer from 8 to 30. In some embodiments, p is an integer from 6 to 20. In some embodiments, p is an integer from 6 to 20. In some embodiments, p is an integer from 8 to 20. In some embodiments, p is an integer from 10 to 30. In some embodiments, p is an integer from 10 to 20. In some embodiments, p is an integer from 1 to 25, 4 to 20, 5 to 15, 6 to 12, or 5 to 10. In some embodiments, p is an integer from 1 to 10, 4 to 10, 6 to 10, or 7 to 9. In some embodiments, p is 8.

In some embodiments, the linker (e.g. L_{A}) comprises or consists of the formula:

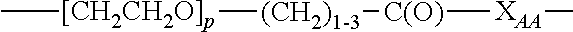

wherein X_{AA} is an amino acid sequence, and p is an integer from 0 to 50, wherein X_{AA} is not Val-Cit or Phe-Lys. In some embodiments, p is an integer from 1 to 40. In some embodiments, p is an integer from 1 to 30. In some embodiments, p is an integer from 6 to 40. In some embodiments, p is an integer from 8 to 30. In some embodiments, p is an integer from 6 to 20. In some embodiments, p is an integer from 6 to 20. In some embodiments, p is an integer from 8 to 20. In some embodiments, p is an integer from 10 to 30. In some embodiments, p is an integer from 10 to 20. In some embodiments, p is an integer from 1 to 25, 4 to 20, 5 to 15, 6 to 12, or 5 to 10. In some embodiments, p is an integer from 1 to 10, 4 to 10, 6 to 10, or 7 to 9. In some embodiments, p is 8.

In some embodiments, the linker (e.g. L_{A}) comprises or consists of the formula:

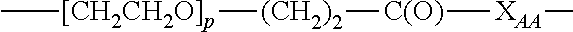

wherein X_{AA} is an amino acid sequence, and p is an integer from 0 to 50. In some embodiments, p is an integer from 1 to 40. In some embodiments, p is an integer from 1 to 30. In some embodiments, p is an integer from 6 to 40. In some embodiments, p is an integer from 8 to 30. In some embodiments, p is an integer from 6 to 20. In some embodiments, p is an integer from 6 to 20. In some embodiments, p is an integer from 8 to 20. In some embodiments, p is an integer from 10 to 30. In some embodiments, p is an integer from 10 to 20. In some embodiments, p is an integer from 1 to 25, 4 to 20, 5 to 15, 6 to 12, or 5 to 10. In some embodiments, p is an integer from 1 to 10, 4 to 10, 6 to 10, or 7 to 9. In some embodiments, p is 8.

In some embodiments, the linker (e.g. L_{A}) comprises or consists of the formula:

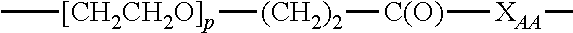

wherein X_{AA} is an amino acid sequence, and p is an integer from 0 to 50, wherein X_{AA} is not Val-Cit or Phe-Lys. In some embodiments, p is an integer from 1 to 40. In some embodiments, p is an integer from 1 to 30. In some embodiments, p is an integer from 6 to 40. In some embodiments, p is an integer from 8 to 30. In some embodiments, p is an integer from 6 to 20. In some embodiments, p is an integer from 6 to 20. In some embodiments, p is an integer from 8 to 20. In some embodiments, p is an integer from 10 to 30. In some embodiments, p is an integer from 10 to 20. In some embodiments, p is an integer from 1 to 25, 4 to 20, 5 to 15, 6 to 12, or 5 to 10. In some embodiments, p is an integer from 1 to 10, 4 to 10, 6 to 10, or 7 to 9. In some embodiments, p is 8.

In some embodiments, a suitable number of ethylene glycol units can be used in the linker. In some embodiments, the linker includes 1, 2, 3, 4, 5, 6, 7, 8, 9, 10, 11, 12, 15, 16, 19, 20, 23, 24, 35, 36, 37, 48, 49, or more ethylene glycol units. In some embodiments, the linker includes 1 to 10, 4 to 10, 6 to 10, or 7 to 9 ethylene glycol units. In some embodiments, the linker includes 8 ethylene glycol units. Non-limiting examples of commercially available ethylene glycol groups (polyethylene glycol, PEG) suitable in the linker include H₂N-dPEG®₈-C(O)OH, having a discrete (“d”) polyethylene glycol having 8 ethylene glycol repeating units. Non-limiting examples of other discrete PEG units are commercially available and known to one of skill in the art, such as by Advanced ChemTech. In some embodiments, the linker comprises the formula:

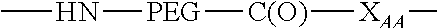

wherein PEG has 1-50 ethylene glycol units, and X_{AA} is an amino acid sequence. In some embodiments, PEG has 1-10 ethylene glycol units, about 4-10 ethylene glycol units, or about 7-9 ethylene glycol units. In some embodiments, PEG has 8 ethylene glycol units.

In some embodiments, the linker comprises the formula:

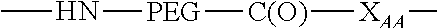

wherein PEG has 1-50 ethylene glycol units, and X_{AA} is an amino acid sequence, with the proviso that X_{AA} is not Val-Cit or Phe-Lys. In some embodiments, PEG has 1-10 ethylene glycol units, about 4-10 ethylene glycol units, or about 7-9 ethylene glycol units. In some embodiments, PEG has 8 ethylene glycol units.

In some embodiments, the linker (e.g. L_{A}) comprises the formula:

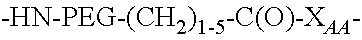

wherein PEG has 1-50 ethylene glycol units, and X_{AA} is an amino acid sequence. In some embodiments, PEG has 1-10 ethylene glycol units, about 4-10 ethylene glycol units, or about 7-9 ethylene glycol units. In some embodiments, PEG has 8 ethylene glycol units. In some embodiments, p is 8.

In some embodiments, the linker (e.g. L_{A}) comprises the formula:

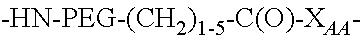

wherein PEG has 1-50 ethylene glycol units, and X_{AA} is an amino acid sequence, with the proviso that X_{AA} is not Val-Cit or Phe-Lys. In some embodiments, PEG has 1-10 ethylene glycol units, about 4-10 ethylene glycol units, or about 7-9 ethylene glycol units. In some embodiments, PEG has 8 ethylene glycol units.

In some embodiments, the linker (e.g. L_{A}) comprises the formula:

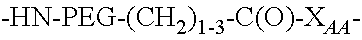

wherein PEG has 1-50 ethylene glycol units, and X_{AA} is an amino acid sequence. In some embodiments, PEG has 1-10 ethylene glycol units, about 4-10 ethylene glycol units, or about 7-9 ethylene glycol units. In some embodiments, PEG has 8 ethylene glycol units. In some embodiments, p is 8.

In some embodiments, the linker (e.g. L_{A}) comprises the formula:

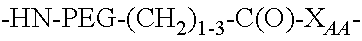

wherein PEG has 1-50 ethylene glycol units, and X_{AA} is an amino acid sequence, with the proviso that X_{AA} is not Val-Cit or Phe-Lys. In some embodiments, PEG has 1-10 ethylene glycol units, about 4-10 ethylene glycol units, or about 7-9 ethylene glycol units. In some embodiments, PEG has 8 ethylene glycol units.

In some embodiments, the linker (e.g. L_{A}) comprises the formula:

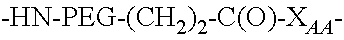

wherein PEG has 1-50 ethylene glycol units, and X_{AA} is an amino acid sequence. In some embodiments, PEG has 1-10 ethylene glycol units, about 4-10 ethylene glycol units, or about 7-9 ethylene glycol units. In some embodiments, PEG has 8 ethylene glycol units.

In some embodiments, the linker (e.g. L_{A}) comprises the formula:

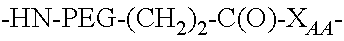

wherein PEG has 1-50 ethylene glycol units, and X_{AA} is an amino acid sequence, with the proviso that X_{AA} is not Val-Cit or Phe-Lys. In some embodiments, PEG has 1-10 ethylene glycol units, about 4-10 ethylene glycol units, or about 7-9 ethylene glycol units. In some embodiments, PEG has 8 ethylene glycol units.

In another non-limiting example, the linker includes an alkylene chain, and/or an amino acid sequence. In some embodiments, the linker comprises the formula:

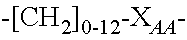

wherein X_{AA} is an amino acid sequence; and the linker include 0, 1, 2, 3, 4, 5, 6, 7, 8, 9, 10, 11 or 12-CH₂— units.

In another non-limiting example, the linker includes an alkylene chain, and/or an amino acid sequence. In some embodiments, the linker comprises the formula:

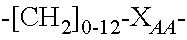

wherein X_{AA} is an amino acid sequence; and the linker include 0, 1, 2, 3, 4, 5, 6, 7, 8, 9, 10, 11 or 12-CH₂— units, with the proviso that X_{AA} is not Val-Cit or Phe-Lys.

In some embodiments, the linker comprises the formula:

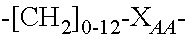

wherein X_{AA} is an amino acid sequence; and the linker include 0, 1, 2, 3, 4, 5, 6, 7, 8, 9, 10, 11 or 12-CH₂— units, with the proviso that X_{AA} is not Val-Cit or Phe-Lys.

In some embodiments, the linker comprises the formula:

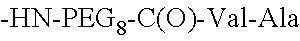

wherein PEGs has 8 ethylene glycol units.

In some embodiments, the linker comprises the formula:

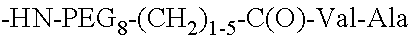

wherein PEGs has 8 ethylene glycol units.

In some embodiments, the linker comprises the formula:

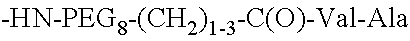

wherein PEGs has 8 ethylene glycol units.

In some embodiments, the linker comprises the formula:

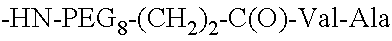

wherein PEGs has 8 ethylene glycol units.

In some embodiments, the linker also includes a variety of other connecting groups that connect the ethylene glycol portion to the amino acid sequence, or connect the ethylene glycol or amino acid sequence to a targeting agent (e.g. an antibody or antibody-binding fragment), or the drug moiety. For example, the amino acid sequence can be connected to the drug moiety via a 4-amino benzyl carboxylate group. In some embodiments, the ethylene glycol portion ca be directly linked to a targeting agent (e.g. an antibody or antibody-binding fragment). In some embodiments, the linker comprises or consists of the formula:

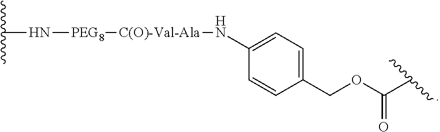

In some embodiments, the HN group is directly linked to a targeting agent (e.g. an antibody or antibody-binding fragment).

In embodiments, the linker is or comprises:

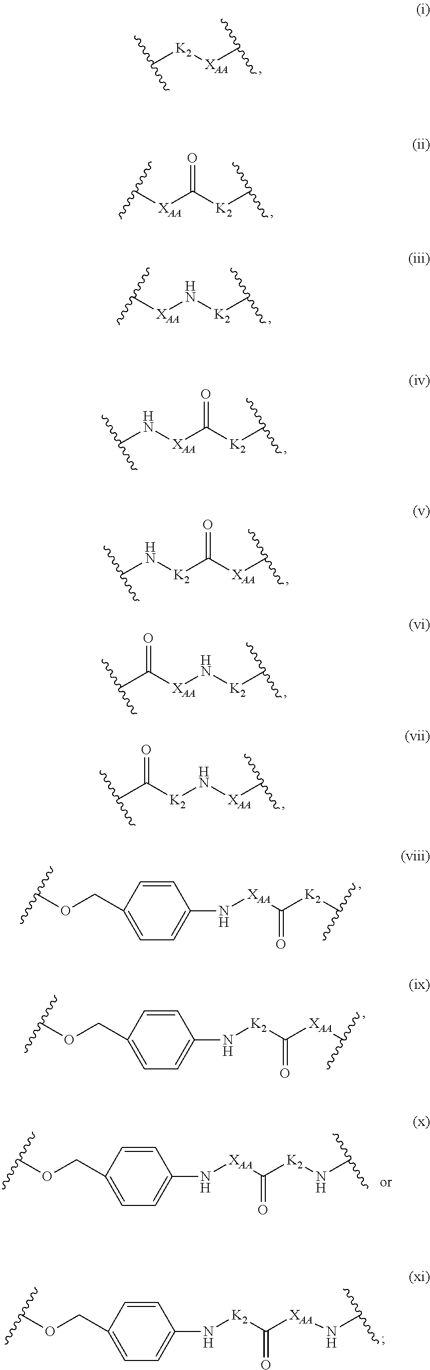

wherein X_{AA} is an amino acid sequence; and K₂ is —[CH₂CH₂O]₀₋₅₀— or —[CH₂]₀₋₁₂—. In some embodiments, the linker is attached to a targeting agent (e.g. an antibody or antibody-binding fragment) and the drug moiety in either direction. In some embodiments, the linker is (i), (ii), (iii), (iv), (vi), (viii) or (ix).

In embodiments, the linker is or comprises:

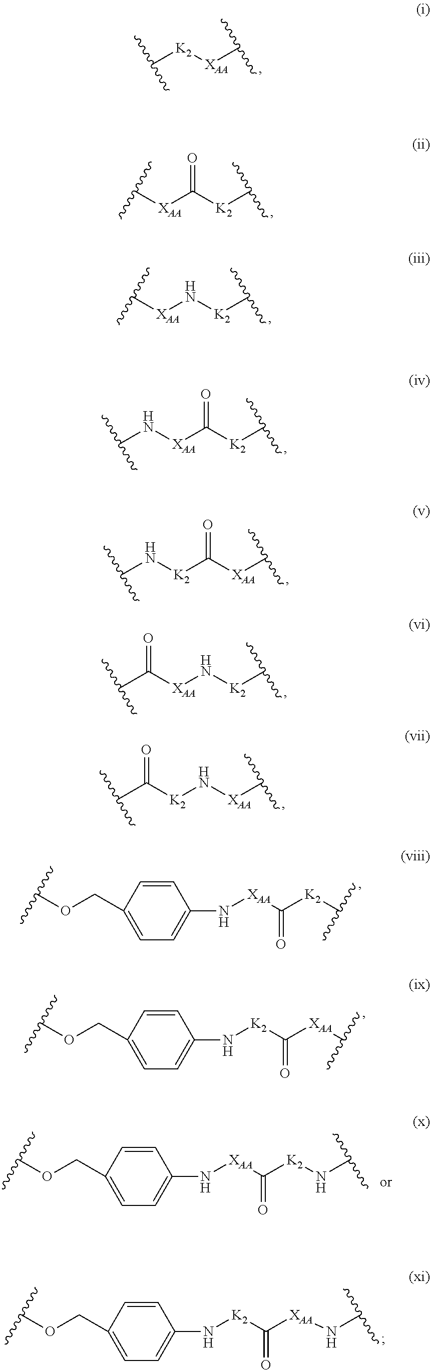

wherein X_{AA} is an amino acid sequence; and K₂ is —[CH₂CH₂O]₀₋₅₀—[CH₂]₀₋₁₂—C(O)—. In some embodiments, the linker is attached to a targeting agent (e.g. an antibody or antibody-binding fragment) and the drug moiety in either direction. In some embodiments, the linker is (i), (ii), (iii), (iv), (vi), (viii) or (ix).

In some embodiments, the linker is or comprises:

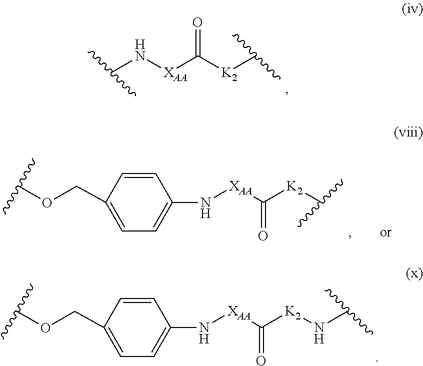

In some embodiments, the linker further comprises 1, 2, 3, 4, 5, 6, 7, 8, 9, 10, 11, 12, 15, 16, 19, 20, 23, 24, 35, 36, 37, 48, 49 or 50 ethylene glycol units. In some embodiments, the linker comprises 0, 1, 2, 3, 4, 5, 6, 7, 8, 9, 10, 11 or 12 —CH₂— units. In some embodiments, the HN group is directly linked to a targeting agent (e.g. an antibody or antibody-binding fragment).

In some embodiments, the linker comprises an amino acid portion which includes any suitable number of amino acid moieties, as described above. In a non-limiting example, the amino acid sequence X_{AA} includes from 1 to 100 amino acid moieties, or from 1 to 10 amino acid moieties, or from 1 to 5 amino acid moieties. In some embodiments, the linker includes 1, 2, 3, 4, 5, 6, 7, 8, 9, 10, or more amino acid moieties. In some embodiments, the linker includes 2 amino acid moieties. In some embodiments, X_{AA} is valine-alanine.

In some embodiments, the amino acid sequence X_{AA} is:

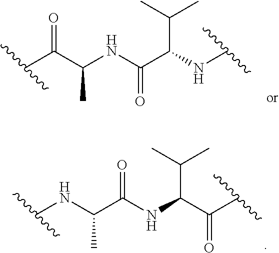 [SMILES: *N[C@H](C(=O)N[C@@H](C)C(*)=O)C(C)C ; *N[C@@H](C)C(=O)N[C@H](C(*)=O)C(C)C]

In some embodiments, the amino acid sequence X_{AA} is:

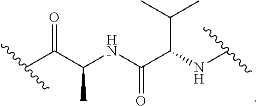 [SMILES: *N[C@H](C(=O)N[C@@H](C)C(*)=O)C(C)C]

In some embodiments, the linker comprises one or more groups selected from C₁-C₆ alkyl, C═O, —NH—, ethylene glycol, optionally 2-10 ethylene glycol units, valine-citrulline (val-cit), 6-maleimidocaproyl (mc), 6-succinimidylcaproyl, 6-(2,5-dioxo-3λ³-pyrrolidin-1-yl)caproyl, methoxy-polyethylene glycol maleimide 6 (MalPeg6), p-aminobenzylcarbamate (PABC), dimethylaminoethanol (DMAE), 3-maleimidopropanoyl (MP), 3-succinimidylpropanoyl, 3-(2,5-dioxo-3λ³-pyrrolidin-1-yl)propanoyl, hydrolyzed Peg-maleimides, hydrolyzed maleimide, hydrolyzed succinimide, valine-alanine (Val-Ala), alanine-phenylalanine (ala-phe), p-aminobenzyloxycarbonyl (PAB), N-Succinimidyl 4-(2-pyridylthio) pentanoate (SPP), N-succinimidyl 4-(N-maleimidomethyl)cyclohexane-1carboxylate (SMCC), N-Succinimidyl (4-iodo-acetyl)aminobenzoate (SIAB), 6-maleimidocaproyl-valine-citrulline-p-aminobenzyloxycarbonyl (mc-val-cit-PAB), and 6-maleimidocaproyl-valine-citrulline-p-aminobenzylcarbamate (mc-val-cit-PABC), an amino acid, optionally (D)-valine, (L)-valine, (D)-alanine, and/or (L)-alanine, and maleimide.

In some embodiments, the linker comprises one or more of C₁-C₆ alkyl, C═O, —NH—, polyethylene glycol (PEG), optionally 2-10 PEG groups, an amino acid, optionally (D)-valine, (L)-valine, (D)-alanine, and/or (L)-alanine, and maleimide, succinimide, or 2,5-dioxo-3λ³-pyrrolidin-1-yl. In some embodiments, the linker comprises and/or consists of valine-citrulline (val-cit). In some embodiments, the linker comprises and/or consists of valine-citrulline (val-cit)-p-aminobenzyloxycarbonyl (PAB).

In some embodiments, the linker comprises one or more reactive moieties capable of reacting with a targeting agent and/or a targeting agent (e.g. an antibody or an antibody fragment). Non-limiting examples of reactive moieties include an azide, alkyne, bisulfone, carbohydrazide, hydrazine, hydroxylamine, iodoacetamide, isothiocyanate, maleimide, phosphine, pyrridopyridazine, semihydrazide, succinimidyl ester, sulfodichlorophenol ester, sulfonyl halide, sulfosuccinimidyl ester, 4-sulfotetrafluorophenyl ester, tetrafluorophenyl ester, thiazole, and NHNH₂. Non-limiting examples of targeting agent include a protein, a portion of a protein, a polypeptide, a nucleic acid, a hormone, an antibody or an antibody fragment. In some embodiments, the targeting agent is an antibody or an antibody fragment.

In some embodiments, the linker comprises R*, where R* is a reactive moiety capable of reacting with a targeting agent, a linking moiety connecting the linker to a targeting agent, or is a targeting agent. In some embodiments, the linker comprises or consists of the following formula:

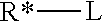

wherein R* is a reactive moiety, a linking moiety, or a targeting agent.

In some embodiments, R* is a reactive moiety, and capable of reacting with functional groups such as aldehydes, amines, disulfides, ketones, thiols in the targeting agent, or in Staudinger reactions, Pictet-Spengler reactions and/or Click-type chemistry with the targeting agent. For some reactive moieties suitable coupling reagents are used to react the reactive moiety with a targeting agent, e.g. where R* is a carboxylic acid, carbodiimide coupling reagents maybe used. In some embodiments, R* is selected from an azide, alkynes, bisulfone, carbohydrazide, hydroxylamine, iodoacetamide, isothiocyanate, maleimide, phosphine, semihydrazide, succinimidyl ester and sulfonyl halide.

In some embodiments, R* is or comprises maleimide:

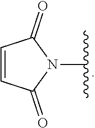 [SMILES: *N1C(=O)C=CC1=O]

In some embodiments, R* is or comprises bisulfone. In some embodiments, the bisulfone is or comprises

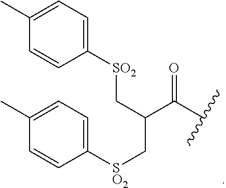 [SMILES: *C(=O)C(CS(=O)(=O)c1ccc(C)cc1)CS(=O)(=O)c1ccc(C)cc1]

In some embodiments, the bisulfone is or comprises

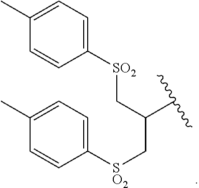 [SMILES: *C(CS(=O)(=O)c1ccc(C)cc1)CS(=O)(=O)c1ccc(C)cc1]

In some embodiments, R* is or comprises an azide, alkynes, bisulfone, carbohydrazide, hydroxylamine, iodoacetamide, isothiocyanate, maleimide, phosphine, semihydrazide, succinimidyl ester and sulfonyl halide.

Non-limiting examples of other chemistries are known for attachment of compounds to antibodies. U.S. Pat. No. 7,595,292 (Brocchini et al.) refers to linkers that form thioesters with the sulfurs in a disulfide bond of an antibody. U.S. Pat. No. 7,985,783 (Carico et al.) refers to the introduction of aldehyde residues into antibodies, which are used to couple compounds to the antibody, all of which are incorporated by reference herein in their entireties.

In some embodiments, R* is a targeting agent wherein the targeting agent is selected from a protein, a portion of a protein, a peptide, a nucleic acid, a hormone, an antibody or an antibody fragment. In some embodiments, the targeting agent binds to a tumor-associated antigen, a cancer-stem-cell associated antigen or a viral antigen.

In some embodiments, the targeting agent is selected from a protein, a portion of a protein, a polypeptide, a nucleic acid, an antibody or an antibody fragment. In some embodiments, the targeting agent is an antibody or an antibody fragment. In some embodiments, the targeting agent is an antibody.

In various embodiments, the targeting agent may bind to a target selected from an acute myeloid leukemia (AML M4) cell, an acute promyelocytic leukemia cell, an acute lymphoblastic leukemia cell, an acute lymphocytic leukemia cell, a chronic lymphocytic leukemia cell, a chronic myeloid leukemia cell, a chronic T-cell lymphocytic leukemia, a myelodysplasia syndromic cell, a multiple myeloma cell, a prostate carcinoma cell, a renal cell adenocarcinoma cell, a pancreatic adenocarcinoma cell, a lung carcinoma cell or a gastric adenocarcinoma cell, a gastric adenocarcinoma cell, a breast cancer cell, a colon cancer cell, a melanoma cell, a thyroid cancer cell, an ovarian cancer cell, a bladder cancer cell, a liver cancer cell, a head and neck cancer cell, an esophageal cancer cell, a hodgkin lymphoma cell, a non-hodgkin lymphoma cell, a mesothelioma cell, a neuroblastoma cell, a neuroendocrine tumor cell, a neurofibromatosis type 1 (NF1) cell, a neurofibromatosis type 2 (NF2) or an osteosarcoma cell.

In some embodiments, the reactive moiety and/or targeting agent further comprises a linking moiety. In some embodiments, the linking moiety is attached to the reactive moiety and/or targeting agent and the linker to connect the reactive moiety and/or targeting agent to the linker. In some embodiments, the linking moiety comprises one or more groups selected from —[CH₂]₀₋₁₂, —[CH₂CH₂O]₀₋₅₀— and —[CH₂]₀₋₁₂—C(O)NH—.

In some embodiments, the linker comprises or consists of the following formula:

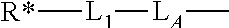

wherein L_{A} is a linker, L₁ is a linking moiety, and R* is a reactive moiety or a targeting agent. In some embodiments, the linker L_{A} is conjugated to the drug moiety.

In some embodiments, linker L_{A} comprises or consists of the formula:

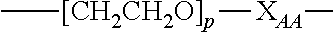

wherein X_{AA} is an amino acid sequence, and p is an integer from 0 to 50. In some embodiments, the linker L_{A} further comprises

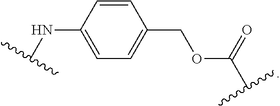 [SMILES: *Nc1ccc(COC(*)=O)cc1]

In some embodiments, linker L_{A} comprises or consists of the formula:

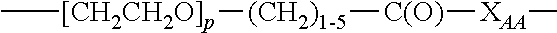

wherein X_{AA} is an amino acid sequence, and p is an integer from 0 to 50. In some embodiments, the linker L_{A} further comprises

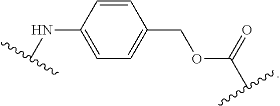 [SMILES: *Nc1ccc(COC(*)=O)cc1]

In some embodiments, linker L_{A} comprises or consists of the formula:

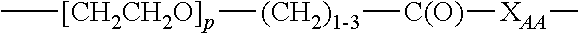

wherein X_{AA} is an amino acid sequence, and p is an integer from 0 to 50. In some embodiments, the linker L_{A} further comprises

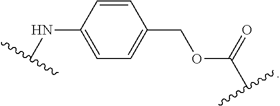 [SMILES: *Nc1ccc(COC(*)=O)cc1]

In some embodiments, linker L_{A} comprises or consists of the formula:

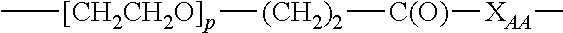

wherein X_{AA} is an amino acid sequence, and p is an integer from 0 to 50. In some embodiments, the linker L_{A} further comprises

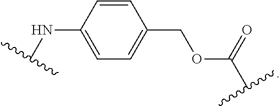 [SMILES: *Nc1ccc(COC(*)=O)cc1]

In some embodiments, R* is a reactive moiety. In some embodiments, the reactive moiety is maleimide. In some embodiments, the reactive moiety is bisulfone.

In some embodiments, L₁ is comprises one or more groups selected from —[CH₂]₀₋₁₂, —[CH₂CH₂O]₀₋₅₀— and —[CH₂]₀₋₁₂—C(O)NH—. In some embodiments, L₁ is —[CH₂]₀₋₁₂—C(O)NH—. In some embodiments, L₁ is —[CH₂]₂—C(O)NH—. In some embodiments, —[CH₂]₅—C(O)NH—.

In some embodiments, L₁ is —[CH₂]₁₋₃—C(O)NH—.

In some embodiments, L₁ further comprises a linking moiety, which is produced from the reaction of a reactive moiety and a functional group such as aldehydes, amines, disulfides, ketones thiols in the targeting agent, or in Staudinger reactions, Pictet-Spengler reactions and/or Click-type chemistry of the targeting agent. In some embodiments, L₁ further comprises a linking moiety selected from a triazole, an amide, a thioether, and a succinimide.

In some embodiments, L₁ further comprises succinimide (i.e., a succinimidyl moiety, “2,5-dioxo-3λ³-pyrrolidin-1-yl”):

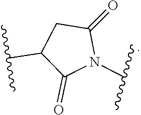 [SMILES: *C1CC(=O)N(*)C1=O]

In some embodiments, L₁ is or comprises

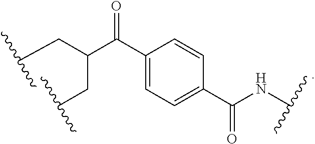 [SMILES: *CC(C*)C(=O)c1ccc(C(=O)N*)cc1]

In some embodiments, L₁ is or comprises

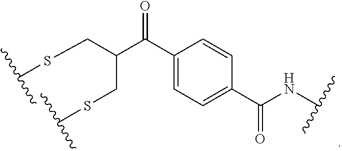 [SMILES: *NC(=O)c1ccc(C(=O)C(CS*)CS*)cc1]

In some embodiments, L₁ is or comprise

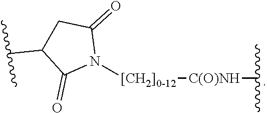

In some embodiments, L₁ is or comprises

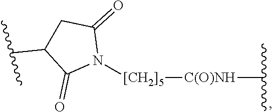

In some embodiments, L₁ is or comprises

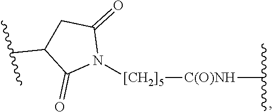

In some embodiments, L₁ further comprises

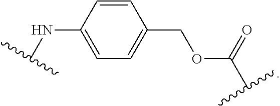 [SMILES: *Nc1ccc(COC(*)=O)cc1]

In some embodiments, R* is a reactive moiety that has reacted with a functional group such as aldehydes, amines, disulfides, ketones thiols in a targeting agent (e.g. with Ab of formula (I)), or in Staudinger reactions, Pictet-Spengler reactions and/or Click-type chemistry of the targeting agent (e.g. with Ab of formula (I)). In some embodiments, R* is selected from succinimide, a triazole, an amide, and a thioether.

In some embodiments, L is a linker of the formula —R*-L₁-L_{A}. In a non-limiting example, R*is a reactive moiety has reacted with a functional group of a targeting agent (e.g. with a cysteine moiety of an antibody or antibody fragment, such as in formula (I)). In some embodiments, R* is selected from succinimide, a triazole, an amide, and a thioether.

In some embodiments, R* is succinimide (i.e., a succinimidyl moiety, “2,5-dioxo-3λ³-pyrrolidin-1-yl”):

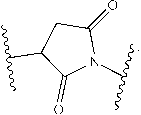 [SMILES: *C1CC(=O)N(*)C1=O]

In some embodiments, R* is

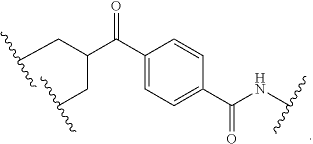 [SMILES: *CC(C*)C(=O)c1ccc(C(=O)N*)cc1]

In some embodiments, R* is

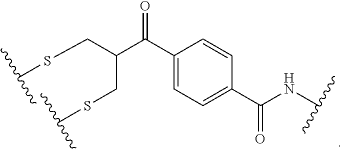 [SMILES: *NC(=O)c1ccc(C(=O)C(CS*)CS*)cc1]

In some embodiments, R* is

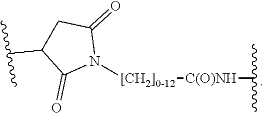

In some embodiments, R* is

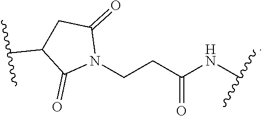 [SMILES: *NC(=O)CCN1C(=O)CC(*)C1=O]

In some embodiments, R* is

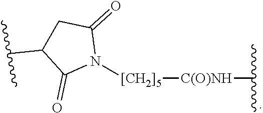

In some embodiments, R* is

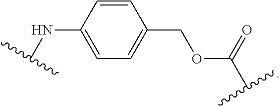 [SMILES: *Nc1ccc(COC(*)=O)cc1]

In some embodiments, the linker comprises or consists of the formula:

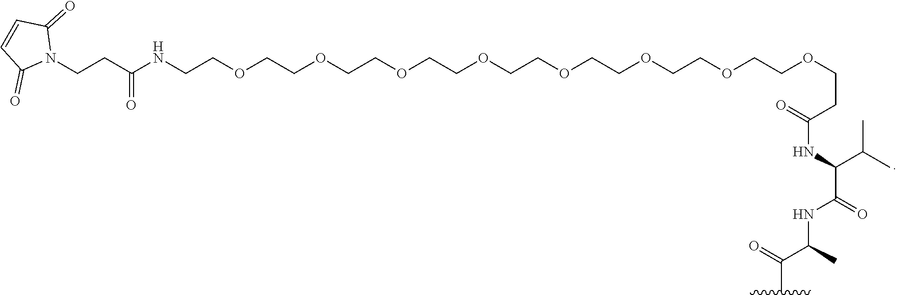 [SMILES: *C(=O)[C@H](C)NC(=O)[C@@H](NC(=O)CCOCCOCCOCCOCCOCCOCCOCCOCCNC(=O)CCN1C(=O)C=CC1=O)C(C)C]

In some embodiments, the linker comprises or consists of the formula:

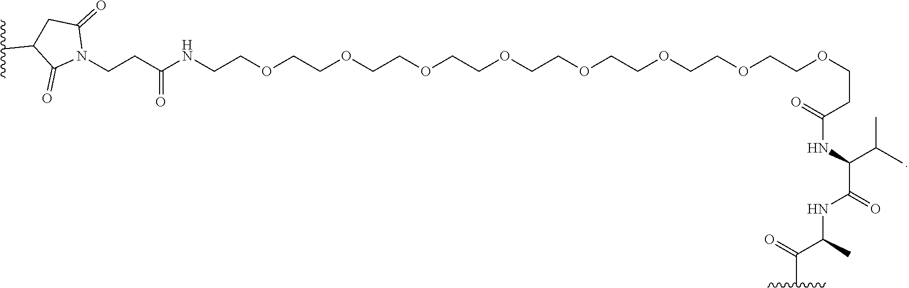 [SMILES: *C(=O)[C@H](C)NC(=O)[C@@H](NC(=O)CCOCCOCCOCCOCCOCCOCCOCCOCCNC(=O)CCN1C(=O)CC(*)C1=O)C(C)C]

In some embodiments, the linker comprises or consists of the formula:

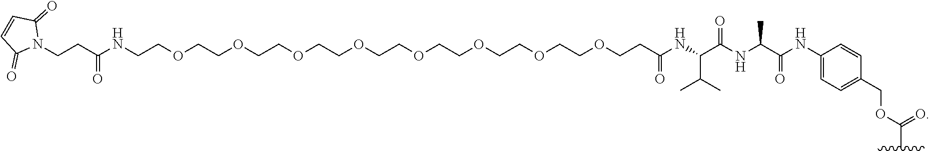 [SMILES: *C(=O)OCc1ccc(NC(=O)[C@H](C)NC(=O)[C@@H](NC(=O)CCOCCOCCOCCOCCOCCOCCOCCOCCNC(=O)CCN2C(=O)C=CC2=O)C(C)C)cc1]

In some embodiments, the linker comprises or consists of the formula:

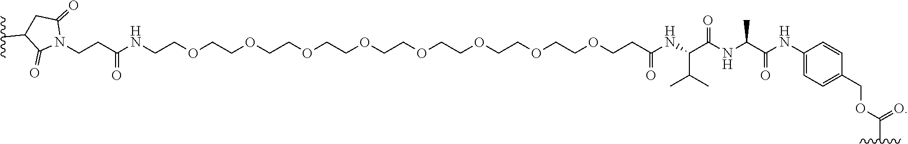 [SMILES: *C(=O)OCc1ccc(NC(=O)[C@H](C)NC(=O)[C@@H](NC(=O)CCOCCOCCOCCOCCOCCOCCOCCOCCNC(=O)CCN2C(=O)CC(*)C2=O)C(C)C)cc1]

In some embodiments, the linker comprises or consists of the formula:

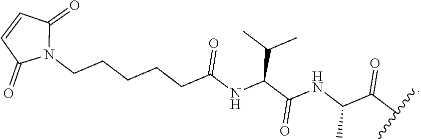 [SMILES: *C(=O)[C@H](C)NC(=O)[C@@H](NC(=O)CCCCCN1C(=O)C=CC1=O)C(C)C]

In some embodiments, the linker comprises or consists of the formula:

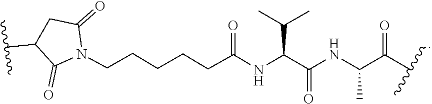 [SMILES: *C(=O)[C@H](C)NC(=O)[C@@H](NC(=O)CCCCCN1C(=O)CC(*)C1=O)C(C)C]

In some embodiments, the linker comprises or consists of the formula:

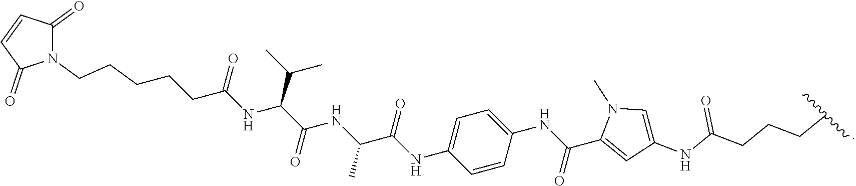 [SMILES: *CCCC(=O)Nc1cc(C(=O)Nc2ccc(NC(=O)[C@H](C)NC(=O)[C@@H](NC(=O)CCCCCN3C(=O)C=CC3=O)C(C)C)cc2)n(C)c1]

In some embodiments, the linker comprises or consists of the formula:

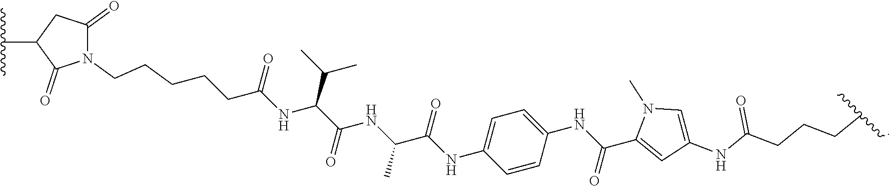 [SMILES: *CCCC(=O)Nc1cc(C(=O)Nc2ccc(NC(=O)[C@H](C)NC(=O)[C@@H](NC(=O)CCCCCN3C(=O)CC(*)C3=O)C(C)C)cc2)n(C)c1]

In some embodiments, the linker comprises or consists of the formula:

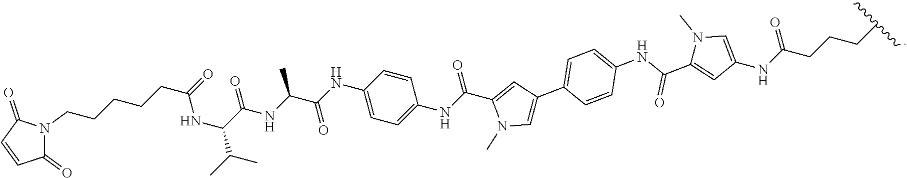 [SMILES: *CCCC(=O)Nc1cc(C(=O)Nc2ccc(-c3cc(C(=O)Nc4ccc(NC(=O)[C@H](C)NC(=O)[C@@H](NC(=O)CCCCCN5C(=O)C=CC5=O)C(C)C)cc4)n(C)c3)cc2)n(C)c1]

In some embodiments, the linker comprises or consists of the formula:

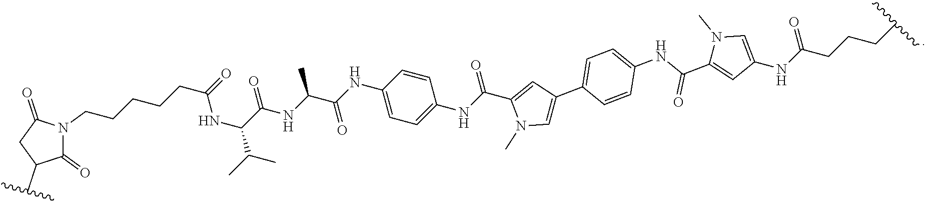 [SMILES: *CCCC(=O)Nc1cc(C(=O)Nc2ccc(-c3cc(C(=O)Nc4ccc(NC(=O)[C@H](C)NC(=O)[C@@H](NC(=O)CCCCCN5C(=O)CC(*)C5=O)C(C)C)cc4)n(C)c3)cc2)n(C)c1]

In some embodiments, the linker comprises or consists of the formula:

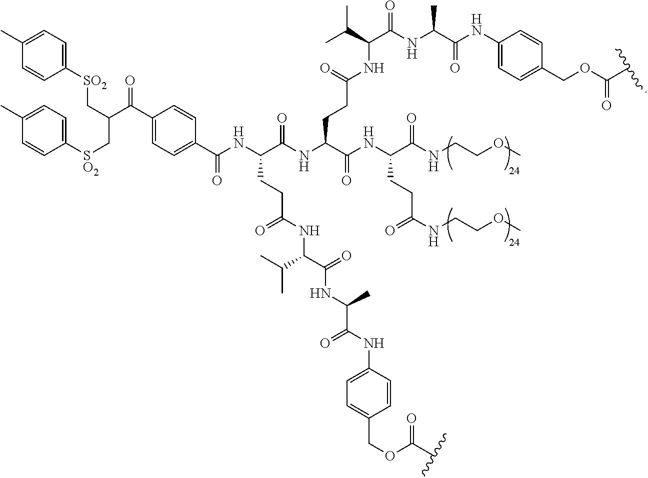

In some embodiments, the linker comprises or consists of the formula:

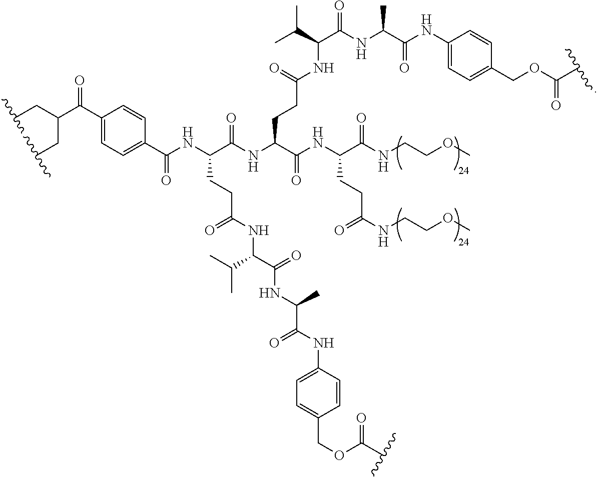

In some embodiments, the linker comprises or consists of the formula:

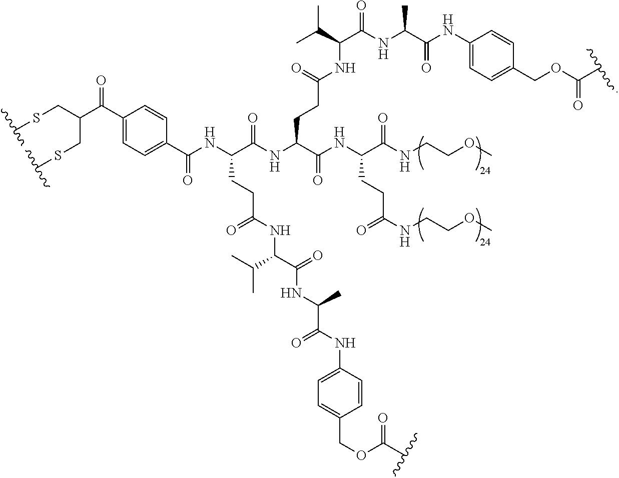

In some embodiments, the linker comprises or consists of the formula:

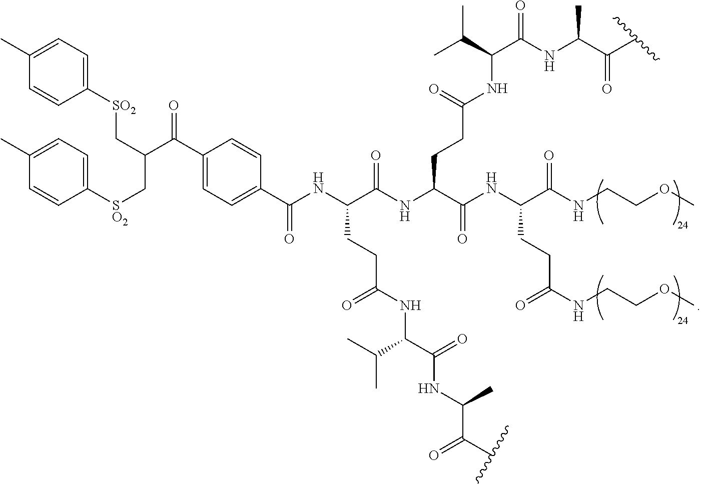

In some embodiments, the linker comprises or consists of the formula:

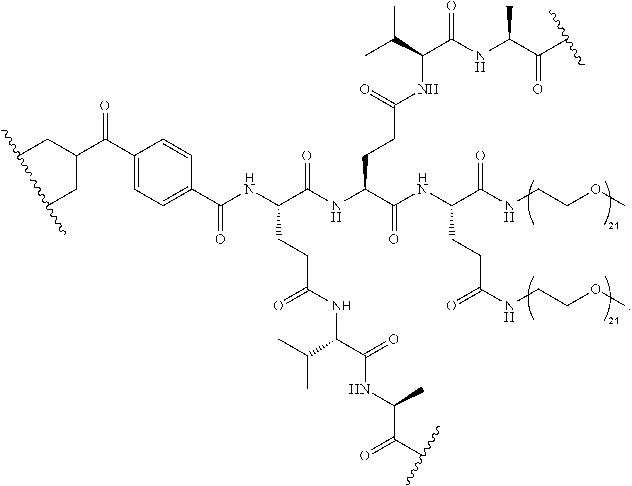

In some embodiments, the linker comprises or consists of the formula:

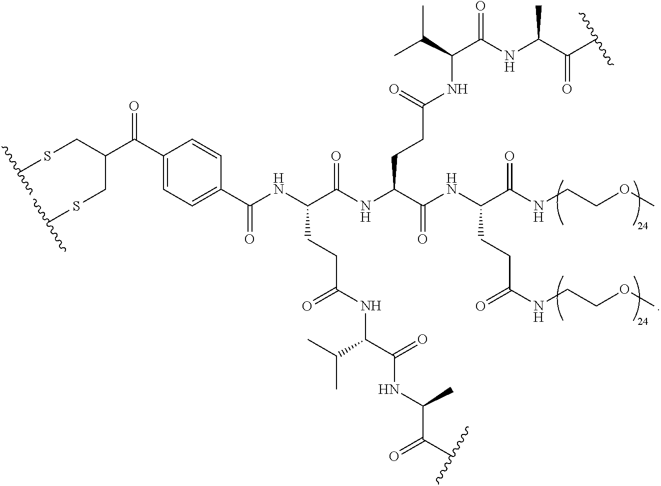

In some embodiments, the linker comprises or consists of the formula:

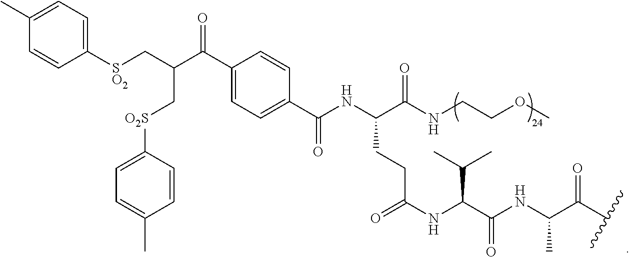

In some embodiments, the linker comprises or consists of the formula:

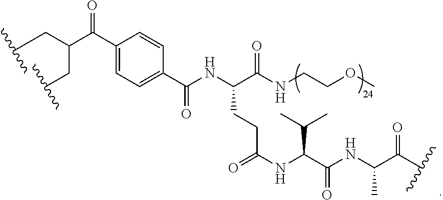

In some embodiments, the linker comprises or consists of the formula:

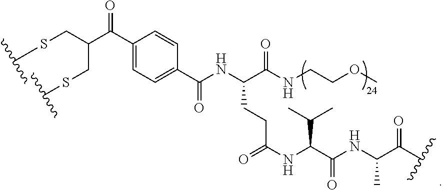

In some embodiments, R* is a targeting agent. In some embodiments, the targeting agent is an antibody or antibody fragment. In some embodiments, the targeting agent is an antibody.

### Compounds

In one aspect, the disclosure provides compounds comprising one or more linkers and one or more drug moieties. In some embodiments, the antibody-drug conjugates of the disclosure (e.g. formula (I)) comprise a compound of formula (III).

In some embodiments, the compound is of formula (III), or salts, solvates, tautomers, isomers or mixtures thereof:

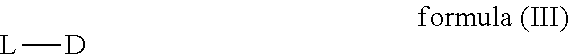

wherein in formula (III):
L is a linker; and
D comprises a drug moiety.

In some embodiments, the linker L of formula (III) reacts with a target moiety (e.g. an antibody or antibody-binding fragment, including but not limited to Ab of formula (III)) that has reacted with the targeting moiety to form a covalent bond with the targeting moiety. In a non-limiting example, the linker L of formula (III) comprises (a reactive group R* which reacts with the antibody or antibody fragment Ab to provide a conjugate of formula (I), wherein the linker L of formula (I) is the linker of formula (III) comprising the product of the reaction of R* with the targeting moiety (e.g. R* is a maleimide in formula (III), and is a succinimide in formula (I), and otherwise L is equivalent in each of formula (I) and formula (III)).

In some embodiments, the antibody is any antibody or antibody fragment disclosed herein. In some embodiments, the antibody or antibody fragment is anti-CDCP1 antibody.

In some embodiments, the drug moiety D is selected from exatecan, Dxd, Sn-38, monomethyl auristatin E (MMAE), and pyrridinobenzodiazepines (PDDs). In some embodiments, the exatecan comprises or has the formula:

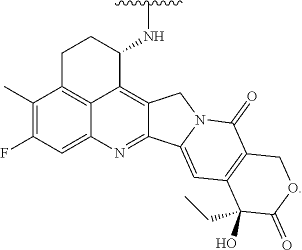 [SMILES: *N[C@H]1CCc2c(C)c(F)cc3nc4c(c1c23)Cn1c-4cc2c(c1=O)COC(=O)[C@]2(O)CC]

In some embodiments, the PDD comprises or has the formula:

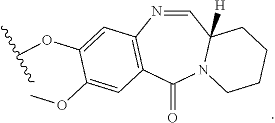 [SMILES: *Oc1cc2c(cc1OC)C(=O)N1CCCC[C@H]1C=N2]

In some embodiments, the PDD comprises or has the formula:

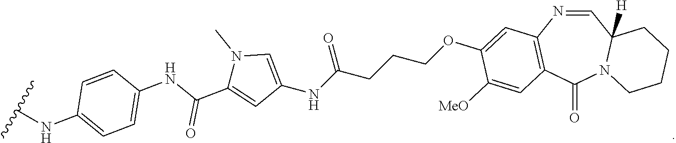 [SMILES: *Nc1ccc(NC(=O)c2cc(NC(=O)CCCOc3cc4c(cc3OC)C(=O)N3CCCC[C@H]3C=N4)cn2C)cc1]

In some embodiments, the PDD comprises or has the formula:

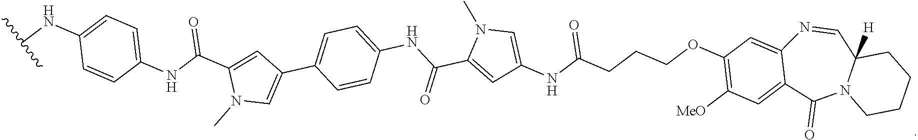 [SMILES: *Nc1ccc(NC(=O)c2cc(-c3ccc(NC(=O)c4cc(NC(=O)CCCOc5cc6c(cc5OC)C(=O)N5CCCC[C@H]5C=N6)cn4C)cc3)cn2C)cc1]

In some embodiments, the Sn-38 comprises or has the formula:

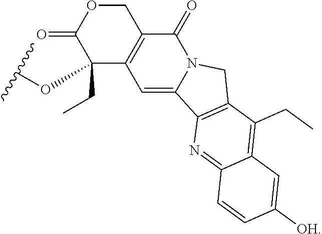 [SMILES: *O[C@]1(CC)C(=O)OCc2c1cc1n(c2=O)Cc2c-1nc1ccc(O)cc1c2CC]

In some embodiments, the compound comprises or consists of the formula:

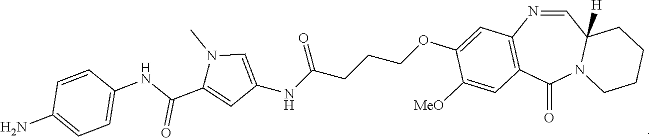 [SMILES: COc1cc2c(cc1OCCCC(=O)Nc1cc(C(=O)Nc3ccc(N)cc3)n(C)c1)N=C[C@@H]1CCCCN1C2=O]

In some embodiments, the compound comprises or consists of the formula:

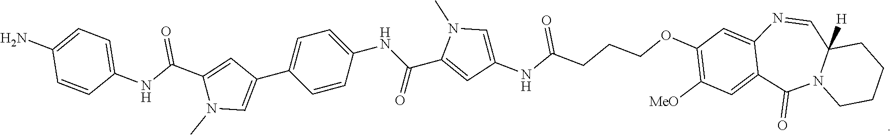 [SMILES: COc1cc2c(cc1OCCCC(=O)Nc1cc(C(=O)Nc3ccc(-c4cc(C(=O)Nc5ccc(N)cc5)n(C)c4)cc3)n(C)c1)N=C[C@@H]1CCCCN1C2=O]

In some embodiments, the linker comprises or consists of the formula:

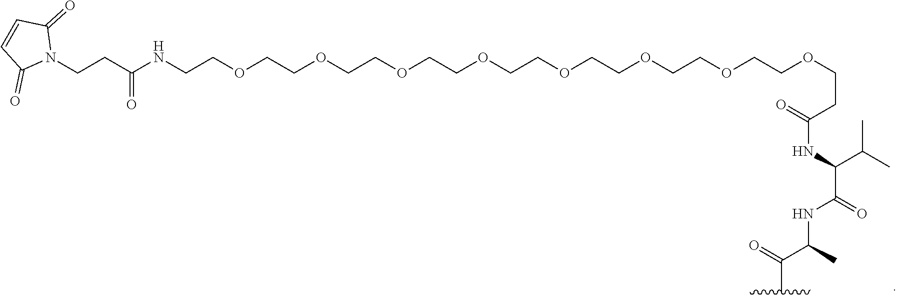 [SMILES: *C(=O)[C@H](C)NC(=O)[C@@H](NC(=O)CCOCCOCCOCCOCCOCCOCCOCCOCCNC(=O)CCN1C(=O)C=CC1=O)C(C)C]

In some embodiments, the linker comprises or consists of the formula:

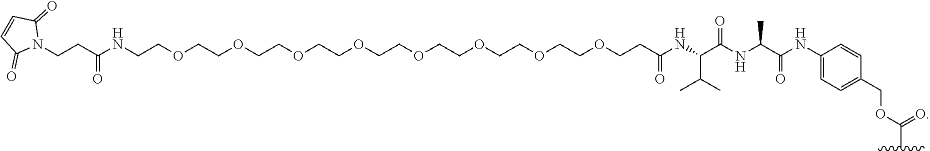 [SMILES: *C(=O)OCc1ccc(NC(=O)[C@H](C)NC(=O)[C@@H](NC(=O)CCOCCOCCOCCOCCOCCOCCOCCOCCNC(=O)CCN2C(=O)C=CC2=O)C(C)C)cc1]

In some embodiments, the linker comprises or consists of the formula:

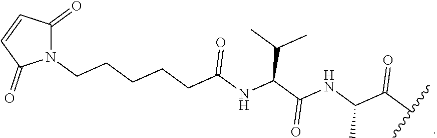 [SMILES: *C(=O)[C@H](C)NC(=O)[C@@H](NC(=O)CCCCCN1C(=O)C=CC1=O)C(C)C]

In some embodiments, the linker comprises or consists of the formula:

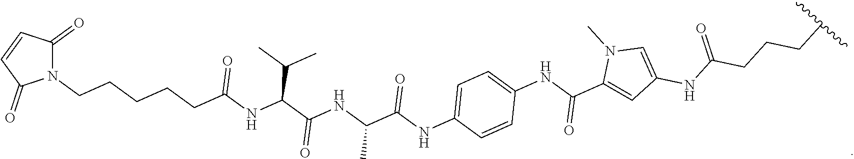 [SMILES: *CCCC(=O)Nc1cc(C(=O)Nc2ccc(NC(=O)[C@H](C)NC(=O)[C@@H](NC(=O)CCCCCN3C(=O)C=CC3=O)C(C)C)cc2)n(C)c1]

In some embodiments, the linker comprises or consists of the formula:

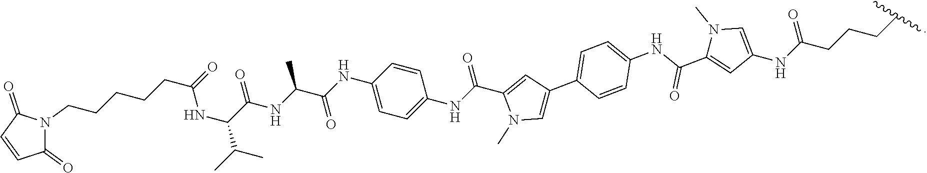 [SMILES: *CCCC(=O)Nc1cc(C(=O)Nc2ccc(-c3cc(C(=O)Nc4ccc(NC(=O)[C@H](C)NC(=O)[C@@H](NC(=O)CCCCCN5C(=O)C=CC5=O)C(C)C)cc4)n(C)c3)cc2)n(C)c1]

In some embodiments, the linker comprises or consists of the formula:

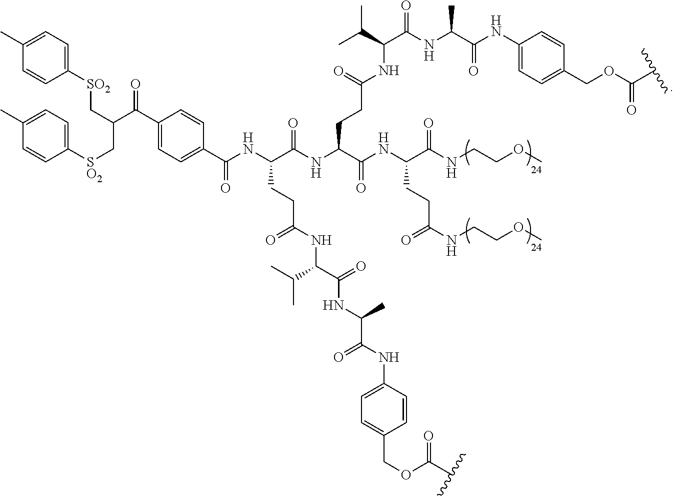

In some embodiments, the linker comprises or consists of the formula:

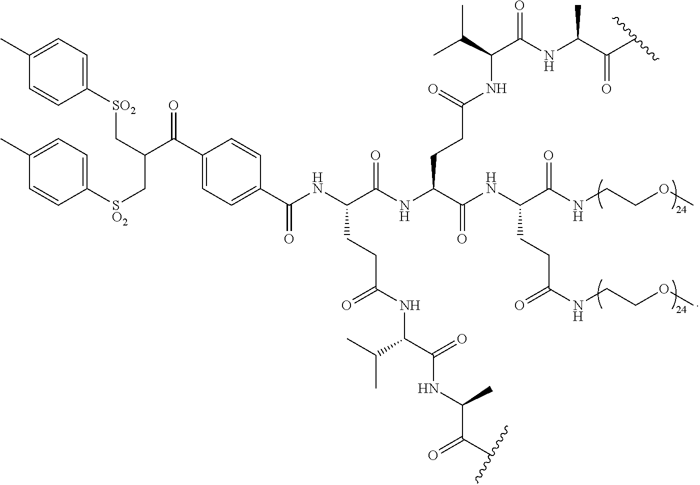

In some embodiments, the linker comprises or consists of the formula:

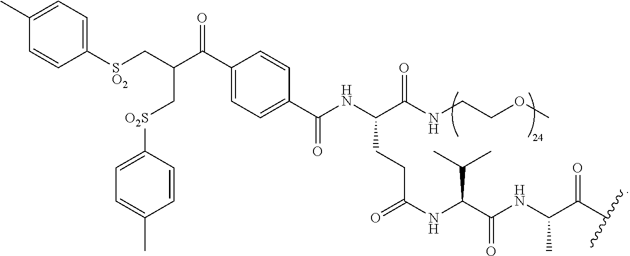

In some embodiments, L-D has the formula:

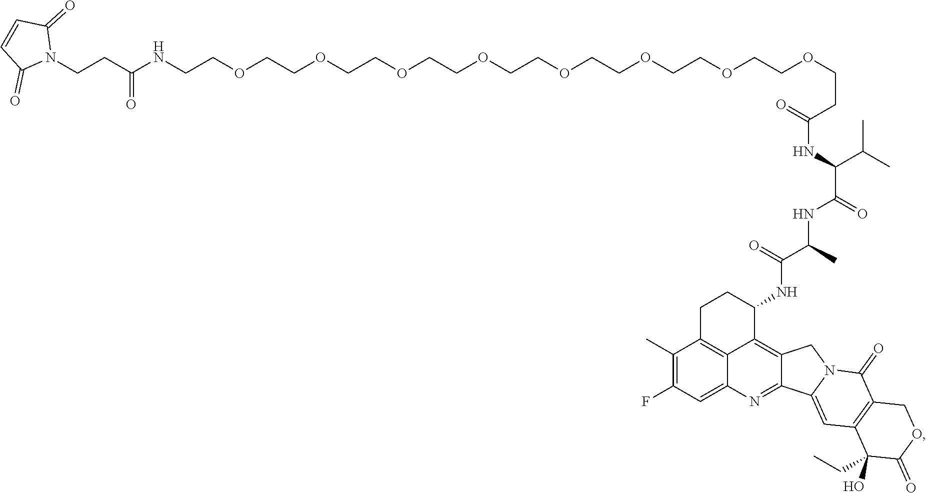 [SMILES: CC[C@@]1(O)C(=O)OCc2c1cc1n(c2=O)Cc2c-1nc1cc(F)c(C)c3c1c2[C@@H](NC(=O)[C@H](C)NC(=O)[C@@H](NC(=O)CCOCCOCCOCCOCCOCCOCCOCCOCCNC(=O)CCN1C(=O)C=CC1=O)C(C)C)CC3]

In some embodiments, L-D has the formula:

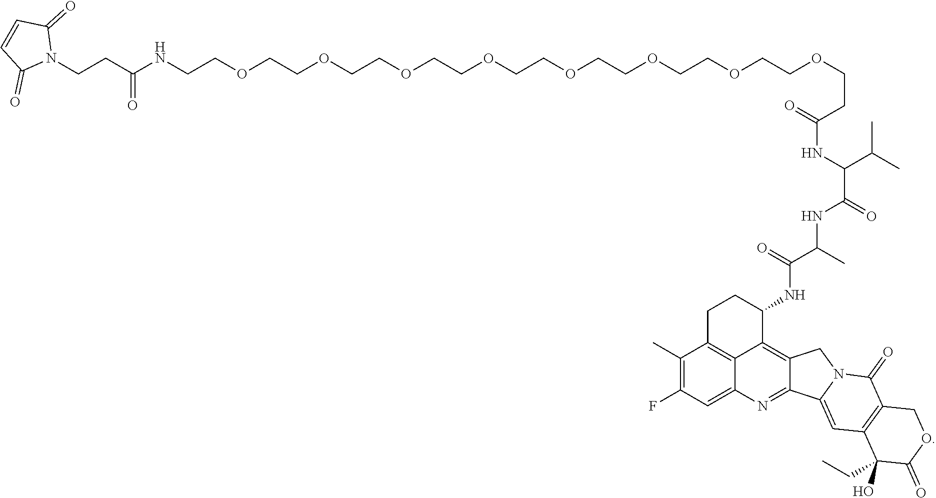 [SMILES: CC[C@@]1(O)C(=O)OCc2c1cc1n(c2=O)Cc2c-1nc1cc(F)c(C)c3c1c2[C@@H](NC(=O)C(C)NC(=O)C(NC(=O)CCOCCOCCOCCOCCOCCOCCOCCOCCNC(=O)CCN1C(=O)C=CC1=O)C(C)C)CC3]

In some embodiments, L-D has the formula:

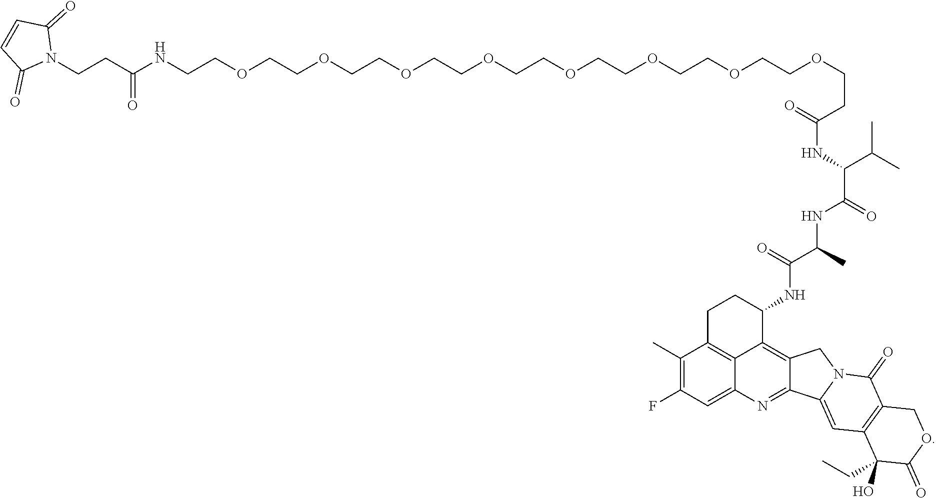 [SMILES: CC[C@@]1(O)C(=O)OCc2c1cc1n(c2=O)Cc2c-1nc1cc(F)c(C)c3c1c2[C@@H](NC(=O)[C@H](C)NC(=O)[C@H](NC(=O)CCOCCOCCOCCOCCOCCOCCOCCOCCNC(=O)CCN1C(=O)C=CC1=O)C(C)C)CC3]

In some embodiments, L-D has the formula:

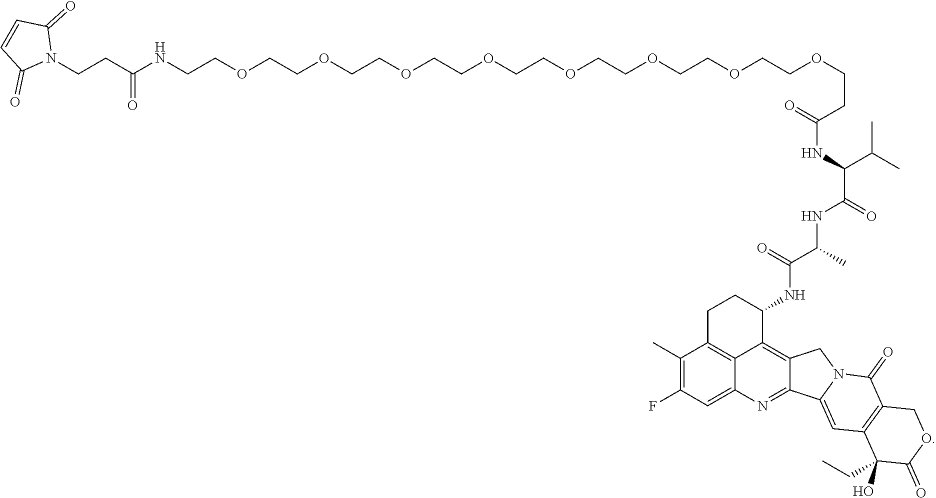 [SMILES: CC[C@@]1(O)C(=O)OCc2c1cc1n(c2=O)Cc2c-1nc1cc(F)c(C)c3c1c2[C@@H](NC(=O)[C@@H](C)NC(=O)[C@@H](NC(=O)CCOCCOCCOCCOCCOCCOCCOCCOCCNC(=O)CCN1C(=O)C=CC1=O)C(C)C)CC3]

In some embodiments, L-D has the formula:

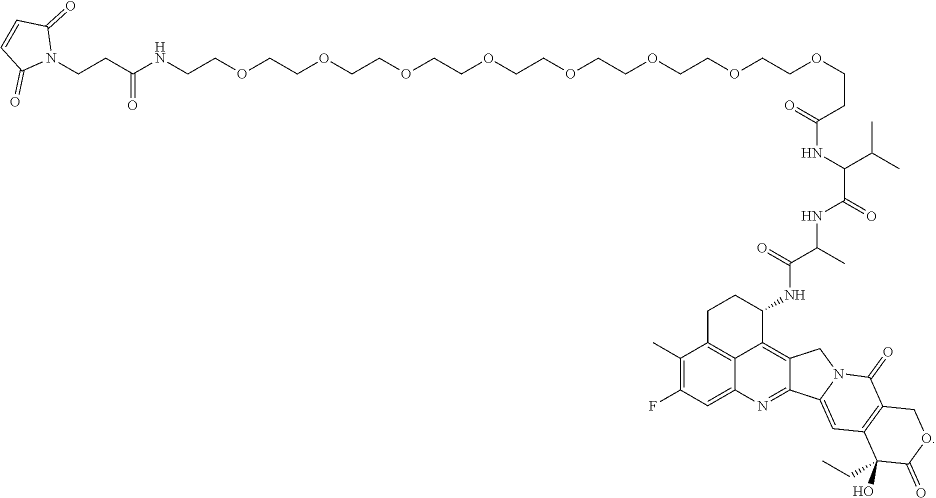 [SMILES: CC[C@@]1(O)C(=O)OCc2c1cc1n(c2=O)Cc2c-1nc1cc(F)c(C)c3c1c2[C@@H](NC(=O)C(C)NC(=O)C(NC(=O)CCOCCOCCOCCOCCOCCOCCOCCOCCNC(=O)CCN1C(=O)C=CC1=O)C(C)C)CC3]

In some embodiments, L-D has the formula:

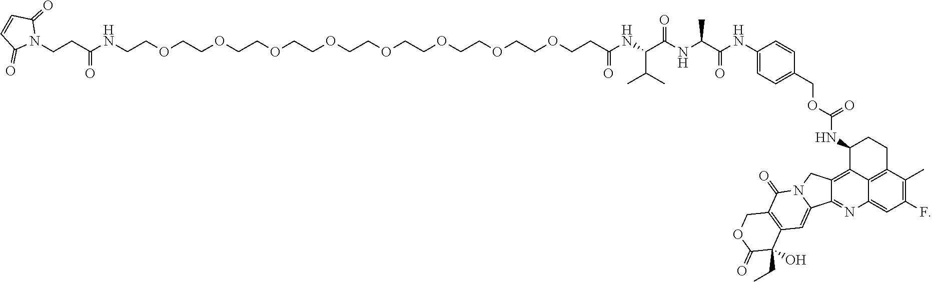 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](C)NC(=O)[C@@H](NC(=O)CCOCCOCCOCCOCCOCCOCCOCCOCCNC(=O)CCN2C(=O)C=CC2=O)C(C)C)cc1)CC3]

In some embodiments, L-D has the formula:

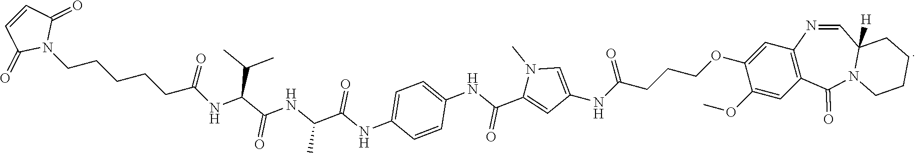 [SMILES: COc1cc2c(cc1OCCCC(=O)Nc1cc(C(=O)Nc3ccc(NC(=O)[C@H](C)NC(=O)[C@@H](NC(=O)CCCCCN4C(=O)C=CC4=O)C(C)C)cc3)n(C)c1)N=C[C@@H]1CCCCN1C2=O]

In some embodiments, L-D has the formula:

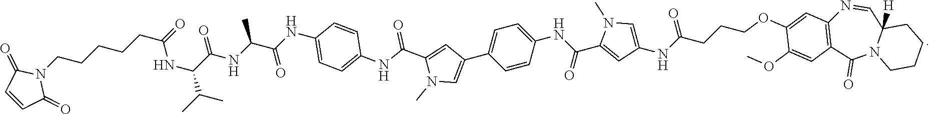 [SMILES: COc1cc2c(cc1OCCCC(=O)Nc1cc(C(=O)Nc3ccc(-c4cc(C(=O)Nc5ccc(NC(=O)[C@H](C)NC(=O)[C@@H](NC(=O)CCCCCN6C(=O)C=CC6=O)C(C)C)cc5)n(C)c4)cc3)n(C)c1)N=C[C@@H]1CCCCN1C2=O]

In some embodiments, L-D has the formula:

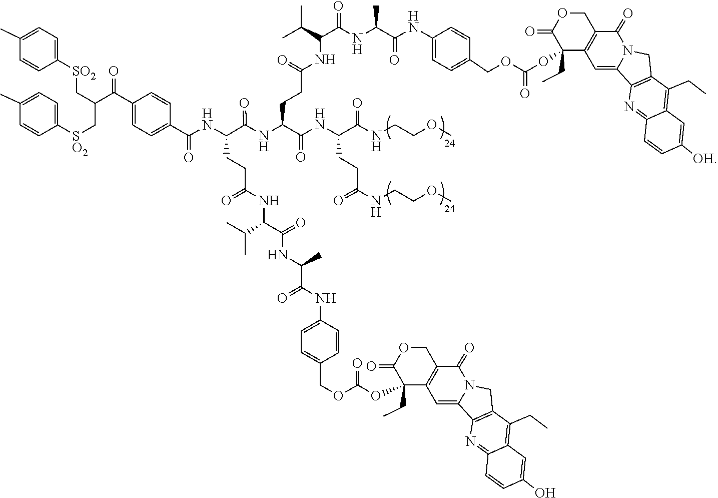

In some embodiments, L-D has the formula:

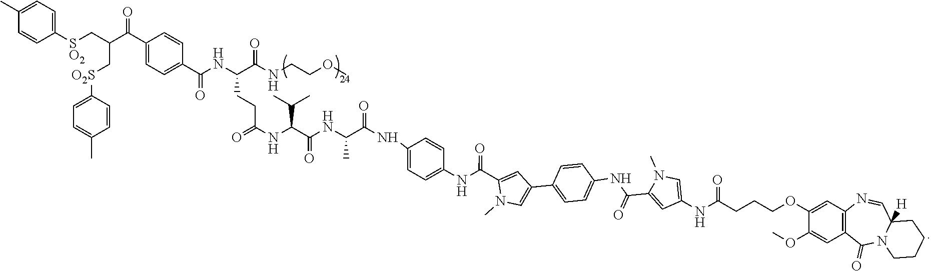

In some embodiments, the compound is of formula (III), or salts, solvates, tautomers, isomers or mixtures thereof:

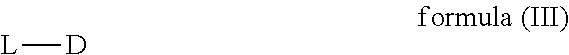

wherein in formula (III):
L is a linker of the formula R*-L₁-L_{A}-;
R* is maleimide;
L₁ is —[CH₂]₁₋₃—C(O)NH—;
L_{A} is —[CH₂CH₂O]ₚ—(CH₂)₁₋₅—C(O)—X_{AA}—, optionally —[CH₂CH₂O]ₚ—(CH₂)₁₋₃—C(O)—X_{AA}—, optionally —[CH₂CH₂O]ₚ—(CH₂)₂—C(O)—X_{AA}—, wherein p is an integer from 5 to 10, and X_{AA} is an amino acid sequence having 2 amino acid moieties; and
D is

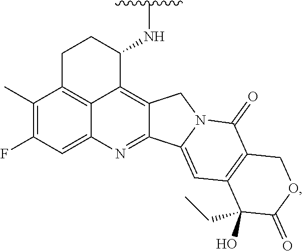 [SMILES: *N[C@H]1CCc2c(C)c(F)cc3nc4c(c1c23)Cn1c-4cc2c(c1=O)COC(=O)[C@]2(O)CC]

### Dxd, or Sn-38.

In some embodiments, the compound is of formula (III), or salts, solvates, tautomers, isomers or mixtures thereof:

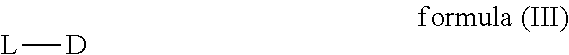

wherein in formula (III):
L is a linker of the formula R*-L₁-L_{A}-;
R* is maleimide;
L₁ is —[CH₂]₁₋₃—C(O)NH—;
L_{A} is —[CH₂CH₂O]ₚ—(CH₂)₁₋₅—C(O)—X_{AA}—, optionally —[CH₂CH₂O]ₚ—(CH₂)₁₋₃—C(O)—X_{AA}—, optionally —[CH₂CH₂O]ₚ—(CH₂)₂—C(O)—X_{AA}—, wherein p is an integer from 5 to 10, and X_{AA} is an amino acid sequence having 2 amino acid moieties; and
D is selected from

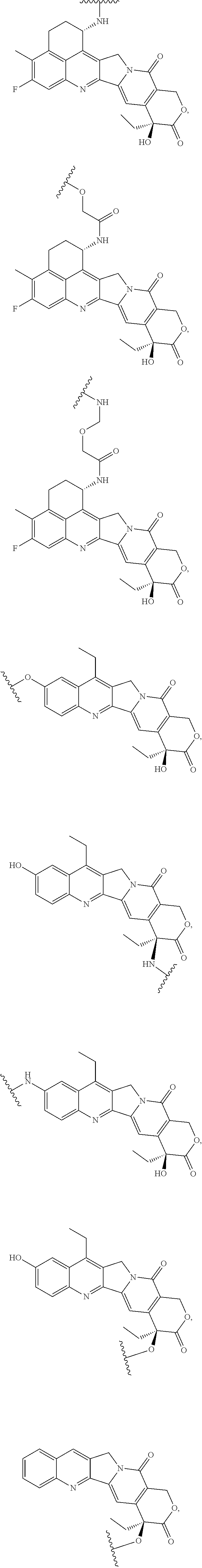

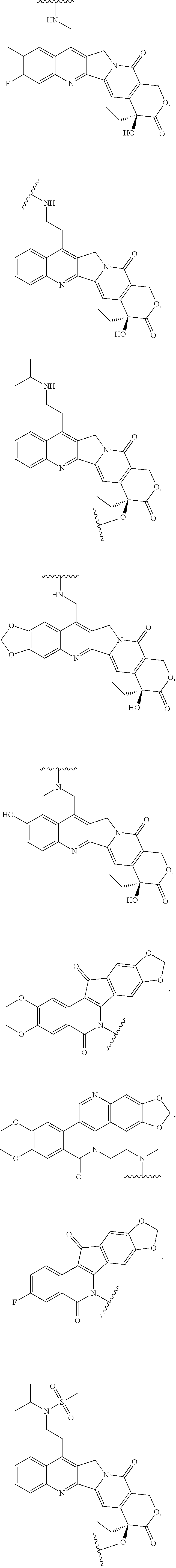

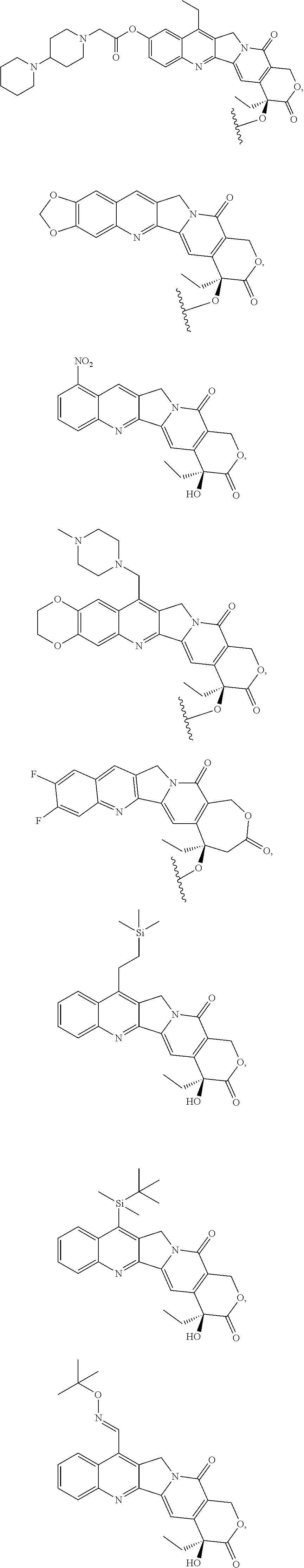

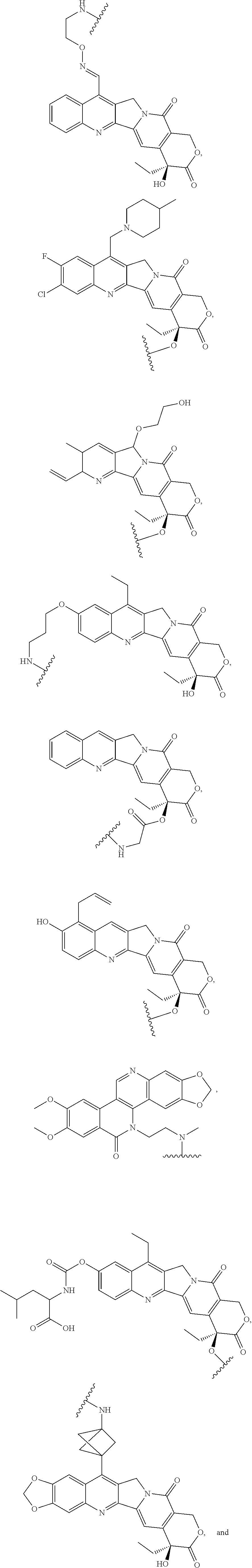

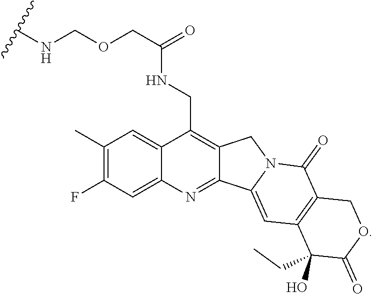 [SMILES: *NCOCC(=O)NCc1c2c(nc3cc(F)c(C)cc13)-c1cc3c(c(=O)n1C2)COC(=O)[C@]3(O)CC]

In some embodiments, X_{AA} is selected from Val-Ala, Tyr-Arg, Phe-Arg, Val-Gln, Val-Cit, Tyr-Met, Leu-Gln, Val-Arg, Met-Thr, Phe-Gln, Thr-Thr, Val-Thr, Ala-Ala, Val-Met, Leu-Met, Ala-Asn, D-Val-D-Gln, D-Ala-D-Ala, and Phe-Met.

In some embodiments, X_{AA} is Val-Ala.

In some embodiments, the compound is of formula (III-A), or salts, solvates, tautomers, isomers or mixtures thereof:

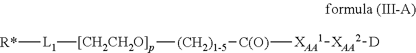

wherein in formula (III-A):
R* is

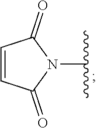 [SMILES: *N1C(=O)C=CC1=O]

L₁ is —[CH₂]₁₋₃—C(O)NH—;
p is an integer from 6 to 20;
X_{AA}¹ and X_{AA}² are independently selected amino acid moieties; and
D is a topoisomerase inhibitor.

In some embodiments, formula (III-A) is has the formula —[CH₂CH₂O]ₚ—(CH₂)₁₋₃. —C(O)—X_{AA}—. In some embodiments, formula (III-A) is has the formula —[CH₂CH₂O]ₚ—(CH₂)₂—C(O)—X_{AA}—.

In some embodiments, X_{AA}¹ is selected from Val, Tyr, Phe, Leu, Met, Thr, Ala, D-Val, and D-Ala. In some embodiments, X_{AA}¹ is Val.

In some embodiments, X_{AA}² is selected from Ala, Arg, Gln, Cit, Met, Thr, Asn, D-Gln, and D-Ala. In some embodiments, X_{AA}² is Ala.

In some embodiments, —X_{AA}¹—X_{AA}²— is selected from Val-Ala, Tyr-Arg, Phe-Arg, Val-Gln, Val-Cit, Tyr-Met, Leu-Gln, Val-Arg, Met-Thr, Phe-Gln, Thr-Thr, Val-Thr, Ala-Ala, Val-Met, Leu-Met, Ala-Asn, D-Val-D-Gln, D-Ala-D-Ala, and Phe-Met.

In some embodiments, —X_{AA}¹—X_{AA}²— is Val-Ala.

In some embodiments, the compound of formula (III) or the compound of formula (III-A) is selected from a compound of any one of formula 30 or 3031-3064, or salts, solvates, tautomers, isomers or mixtures thereof:

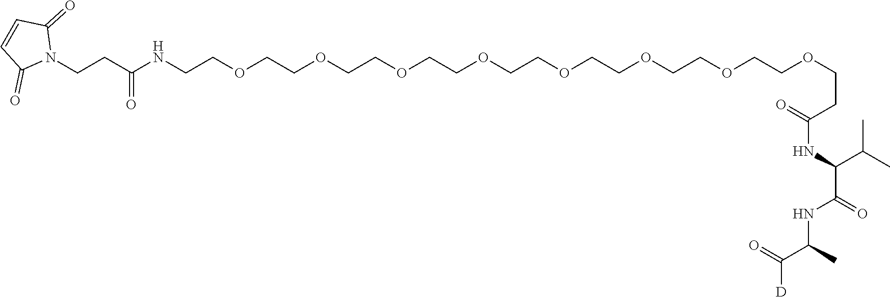 [SMILES: [2H]C(=O)[C@H](C)NC(=O)[C@@H](NC(=O)CCOCCOCCOCCOCCOCCOCCOCCOCCNC(=O)CCN1C(=O)C=CC1=O)C(C)C]

| Formula | D |
|---|---|
| 30 | 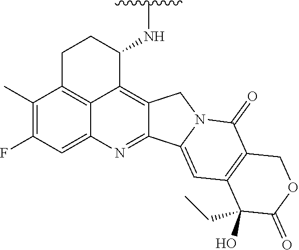 [SMILES: *N[C@H]1CCc2c(C)c(F)cc3nc4c(c1c23)Cn1c-4cc2c(c1=O)COC(=O)[C@]2(O)CC] |
| 3031 | 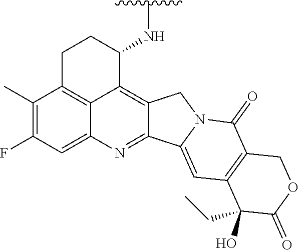 [SMILES: *N[C@H]1CCc2c(C)c(F)cc3nc4c(c1c23)Cn1c-4cc2c(c1=O)COC(=O)[C@]2(O)CC] |
| 3032 | 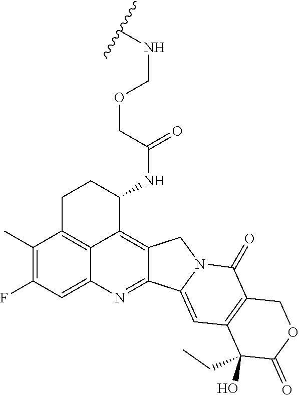 [SMILES: *NCOCC(=O)N[C@H]1CCc2c(C)c(F)cc3nc4c(c1c23)Cn1c-4cc2c(c1=O)COC(=O)[C@]2(O)CC] |
| 3033 | 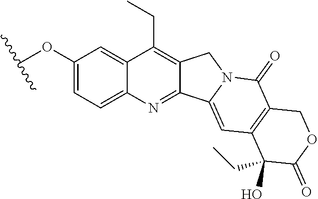 [SMILES: *Oc1ccc2nc3c(c(CC)c2c1)Cn1c-3cc2c(c1=O)COC(=O)[C@]2(O)CC] |
| 3034 | 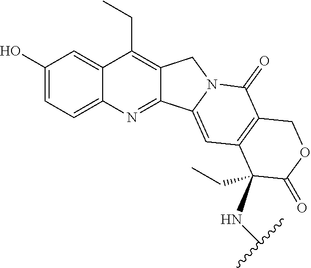 [SMILES: *N[C@]1(CC)C(=O)OCc2c1cc1n(c2=O)Cc2c-1nc1ccc(O)cc1c2CC] |
| 3035 | 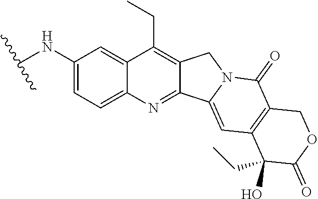 [SMILES: *Nc1ccc2nc3c(c(CC)c2c1)Cn1c-3cc2c(c1=O)COC(=O)[C@]2(O)CC] |
| 3036 | 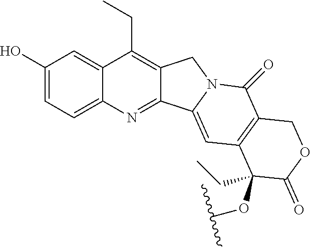 [SMILES: *O[C@]1(CC)C(=O)OCc2c1cc1n(c2=O)Cc2c-1nc1ccc(O)cc1c2CC] |
| 3037 | 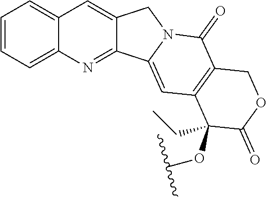 [SMILES: *O[C@]1(CC)C(=O)OCc2c1cc1n(c2=O)Cc2cc3ccccc3nc2-1] |
| 3038 | 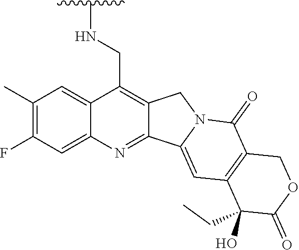 [SMILES: *NCc1c2c(nc3cc(F)c(C)cc13)-c1cc3c(c(=O)n1C2)COC(=O)[C@]3(O)CC] |
| 3039 | 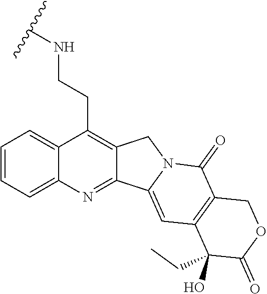 [SMILES: *NCCc1c2c(nc3ccccc13)-c1cc3c(c(=O)n1C2)COC(=O)[C@]3(O)CC] |
| 3040 | 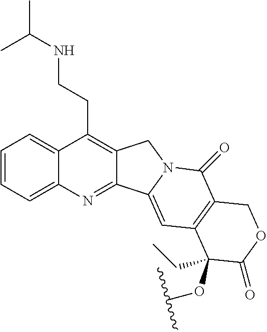 [SMILES: *O[C@]1(CC)C(=O)OCc2c1cc1n(c2=O)Cc2c-1nc1ccccc1c2CCNC(C)C] |
| 3041 | 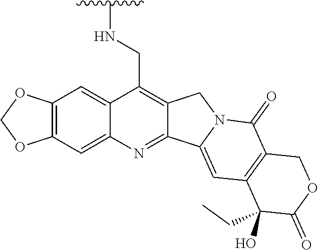 [SMILES: *NCc1c2c(nc3cc4c(cc13)OCO4)-c1cc3c(c(=O)n1C2)COC(=O)[C@]3(O)CC] |
| 3042 | 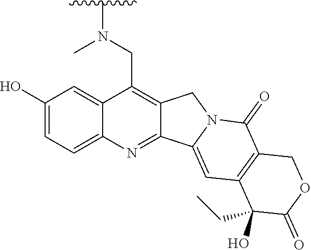 [SMILES: *N(C)Cc1c2c(nc3ccc(O)cc13)-c1cc3c(c(=O)n1C2)COC(=O)[C@]3(O)CC] |
| 3043 | 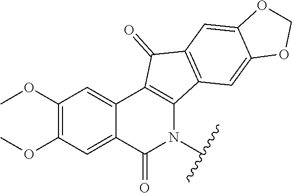 [SMILES: *n1c2c(c3cc(OC)c(OC)cc3c1=O)C(=O)c1cc3c(cc1-2)OCO3] |
| 3044 | 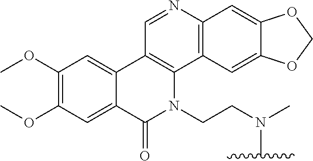 [SMILES: *N(C)CCn1c(=O)c2cc(OC)c(OC)cc2c2cnc3cc4c(cc3c21)OCO4] |
| 3045 | 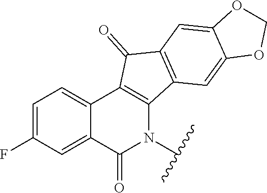 [SMILES: *n1c2c(c3ccc(F)cc3c1=O)C(=O)c1cc3c(cc1-2)OCO3] |
| 3046 | 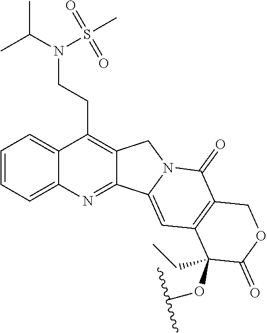 [SMILES: *O[C@]1(CC)C(=O)OCc2c1cc1n(c2=O)Cc2c-1nc1ccccc1c2CCN(C(C)C)S(C)(=O)=O] |
| 3047 | 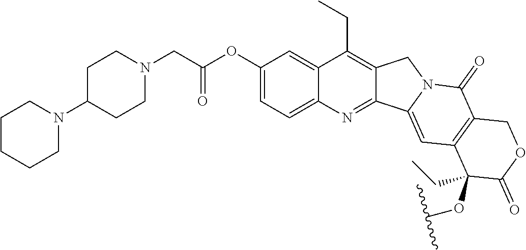 [SMILES: *O[C@]1(CC)C(=O)OCc2c1cc1n(c2=O)Cc2c-1nc1ccc(OC(=O)CN3CCC(N4CCCCC4)CC3)cc1c2CC] |
| 3048 | 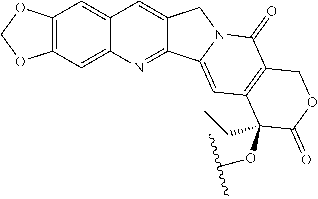 [SMILES: *O[C@]1(CC)C(=O)OCc2c1cc1n(c2=O)Cc2cc3cc4c(cc3nc2-1)OCO4] |
| 3049 | 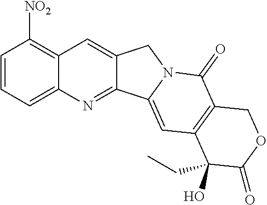 |
| 3050 | 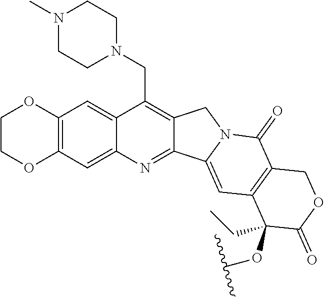 [SMILES: *O[C@]1(CC)C(=O)OCc2c1cc1n(c2=O)Cc2c-1nc1cc3c(cc1c2CN1CCN(C)CC1)OCCO3] |
| 3051 | 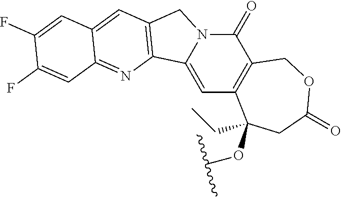 [SMILES: *O[C@]1(CC)CC(=O)OCc2c1cc1n(c2=O)Cc2cc3cc(F)c(F)cc3nc2-1] |
| 3052 | 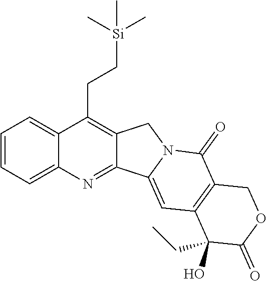 |
| 3053 | 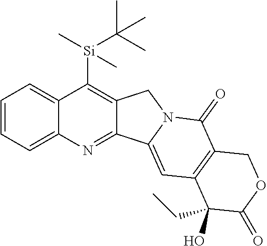 |
| 3054 | 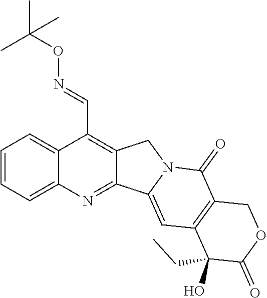 |
| 3055 | 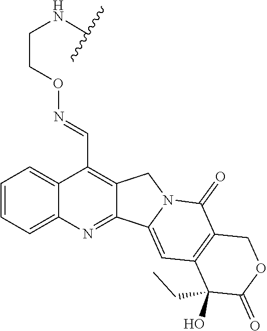 [SMILES: *NCCO/N=C/c1c2c(nc3ccccc13)-c1cc3c(c(=O)n1C2)COC(=O)[C@]3(O)CC] |
| 3056 | 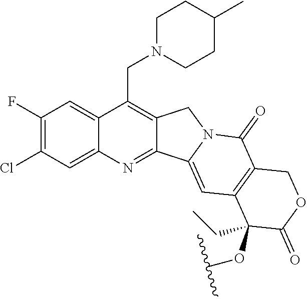 [SMILES: *O[C@]1(CC)C(=O)OCc2c1cc1n(c2=O)Cc2c-1nc1cc(Cl)c(F)cc1c2CN1CCC(C)CC1] |
| 3057 | 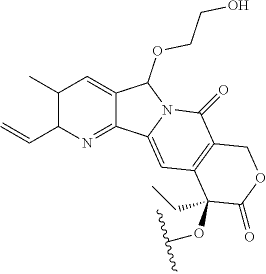 [SMILES: *O[C@]1(CC)C(=O)OCc2c1cc1n(c2=O)C(OCCO)C2=CC(C)C(C=C)N=C21] |
| 3058 | 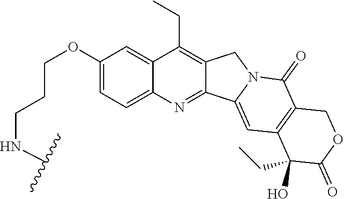 [SMILES: *NCCCOc1ccc2nc3c(c(CC)c2c1)Cn1c-3cc2c(c1=O)COC(=O)[C@]2(O)CC] |
| 3059 | 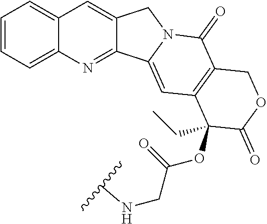 [SMILES: *NCC(=O)O[C@]1(CC)C(=O)OCc2c1cc1n(c2=O)Cc2cc3ccccc3nc2-1] |
| 3060 | 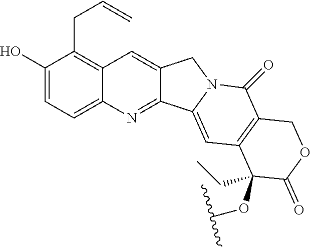 [SMILES: *O[C@]1(CC)C(=O)OCc2c1cc1n(c2=O)Cc2cc3c(CC=C)c(O)ccc3nc2-1] |
| 3061 | 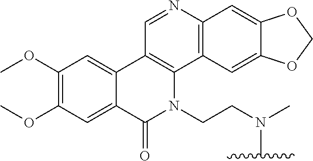 [SMILES: *N(C)CCn1c(=O)c2cc(OC)c(OC)cc2c2cnc3cc4c(cc3c21)OCO4] |
| 3062 | 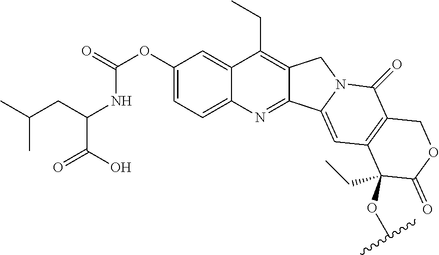 [SMILES: *O[C@]1(CC)C(=O)OCc2c1cc1n(c2=O)Cc2c-1nc1ccc(OC(=O)NC(CC(C)C)C(=O)O)cc1c2CC] |
| 3063 | 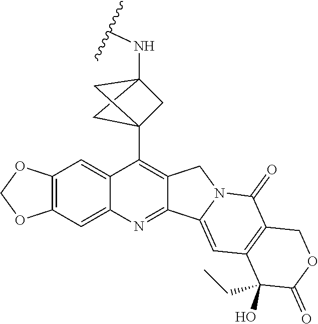 [SMILES: *NC12CC(c3c4c(nc5cc6c(cc35)OCO6)-c3cc5c(c(=O)n3C4)COC(=O)[C@]5(O)CC)(C1)C2] |
| 3064 | 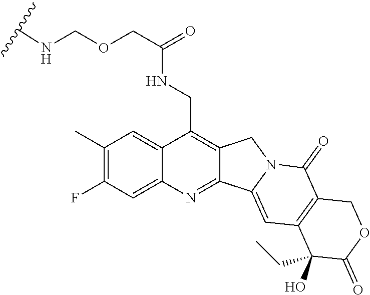 [SMILES: *NCOCC(=O)NCc1c2c(nc3cc(F)c(C)cc13)-c1cc3c(c(=O)n1C2)COC(=O)[C@]3(O)CC] |

In some embodiments, the compound of formula (III) or the compound of formula (III-A) is selected from a compound of any one of formula 30 or 3100-3118, or salts, solvates, tautomers, isomers or mixtures thereof:

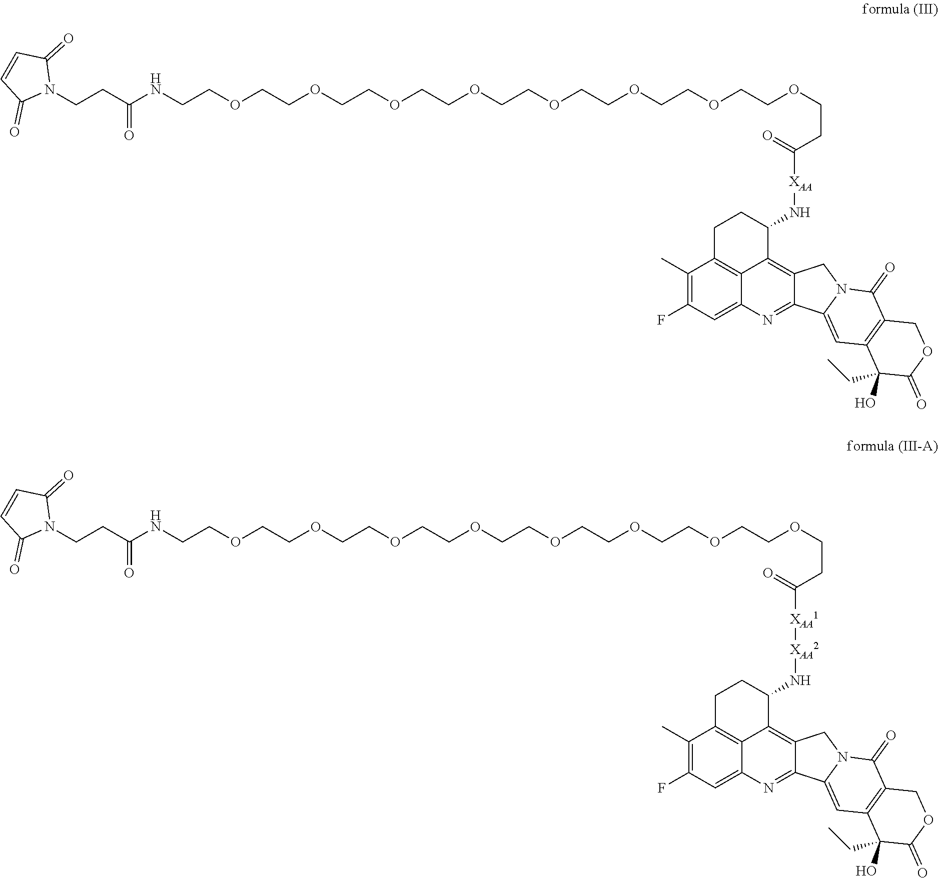

| Formula | -X_{AA}- or -X_{AA}¹-X_{AA}²- |
|---|---|
| 30 | Val-Ala |
| 3101 | Tyr-Arg |
| 3102 | Phe-Arg |
| 3103 | Val-Gln |
| 3104 | Val-Cit |
| 3105 | Tyr-Met |
| 3106 | Leu-Gln |
| 3107 | Val-Arg |
| 3108 | Met-Thr |
| 3109 | Phe-Gln |
| 3110 | Thr-Thr |
| 3111 | Val-Thr |
| 3112 | Ala-Ala |
| 3113 | Val-Met |
| 3114 | Leu-Met |
| 3115 | Ala-Asn |
| 3116 | D-Val-D-Gln |
| 3117 | D-Ala-D-Ala |
| 3118 | Phe-Met |

In one embodiment (CI), the disclosure provides a compound of formula (III), or salts, solvates, tautomers, isomers or mixtures thereof:

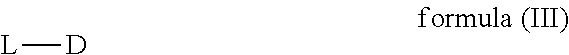

wherein in formula (III):
L is a linker; and
D comprises a drug moiety.
   The linker has the following formula:

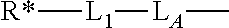

wherein L_{A} is a linker, L₁ is a linking moiety, and R* is a reactive moiety or a targeting agent. The linker L_{A} has the formula:

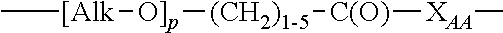

wherein
Alk designates C₂-C₄-alkylene,
X_{AA} is a dipeptide,
p is an integer from 0 to 50.
   R* is a reactive moiety selected from azide, alkynes, bisulfone, carbohydrazide, hydroxylamine, iodoacetamide, isothiocyanate, maleimide, phosphine, semihydrazide, succinimidyl ester and sulfonyl halide. Further, R* is or comprises optionally a bisulfone, optionally the bisulfone is or comprises

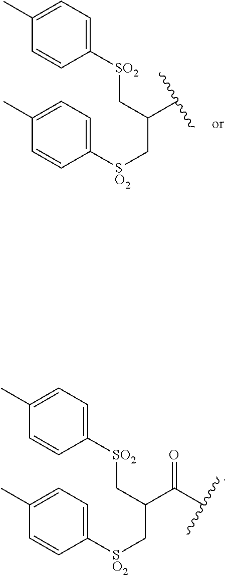 [SMILES: *C(CS(=O)(=O)c1ccc(C)cc1)CS(=O)(=O)c1ccc(C)cc1 ; *C(=O)C(CS(=O)(=O)c1ccc(C)cc1)CS(=O)(=O)c1ccc(C)cc1]

L₁ is —[CH₂]₀₋₁₂—C(O)NH—.
The drug moiety is selected from exatecan having the formula:

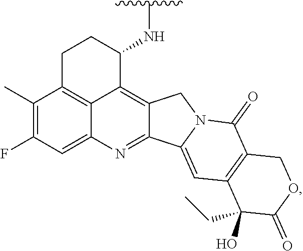 [SMILES: *N[C@H]1CCc2c(C)c(F)cc3nc4c(c1c23)Cn1c-4cc2c(c1=O)COC(=O)[C@]2(O)CC]

Dxd, Sn-38, monomethyl auristatin E (MMAE), and a pyrridinobenzodiazepine (PDD).

In one embodiment (CI), the disclosure provides a compound of formula (III), or salts, solvates, tautomers, isomers or mixtures thereof:

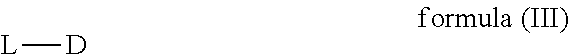

wherein in formula (III):
L is a linker; and
D comprises a drug moiety.
   The linker has the following formula:

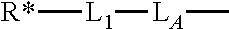

wherein L_{A} is a linker, L₁ is a linking moiety, and R* is a reactive moiety or a targeting agent. The linker L_{A} has the formula:

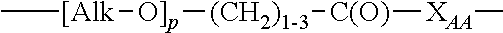

wherein
Alk designates C₂-C₄-alkylene,
X_{AA} is a dipeptide,
p is an integer from 0 to 50.
   R* is a reactive moiety selected from azide, alkynes, bisulfone, carbohydrazide, hydroxylamine, iodoacetamide, isothiocyanate, maleimide, phosphine, semihydrazide, succinimidyl ester and sulfonyl halide. Further, R* is or comprises optionally a bisulfone, optionally the bisulfone is or comprises

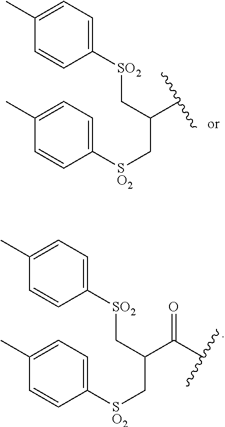 [SMILES: *C(CS(=O)(=O)c1ccc(C)cc1)CS(=O)(=O)c1ccc(C)cc1 ; *C(=O)C(CS(=O)(=O)c1ccc(C)cc1)CS(=O)(=O)c1ccc(C)cc1]

L₁ is —[CH₂]₀₋₁₂—C(O)NH—.
The drug moiety is selected from exatecan having the formula:

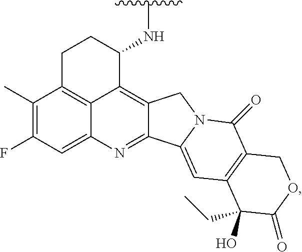 [SMILES: *N[C@H]1CCc2c(C)c(F)cc3nc4c(c1c23)Cn1c-4cc2c(c1=O)COC(=O)[C@]2(O)CC]

Dxd, Sn-38, monomethyl auristatin E (MMAE), and a pyrridinobenzodiazepine (PDD).

In one embodiment (CIb), the disclosure provides a compound of formula (III), or salts, solvates, tautomers, isomers or mixtures thereof:

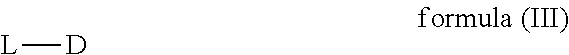

wherein in formula (III):
L is a linker; and
D comprises a drug moiety.
   The linker has the following formula:

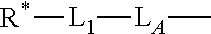

wherein L_{A} is a linker, L₁ is a linking moiety, and R* is a reactive moiety or a targeting agent. The linker L_{A} has the formula:

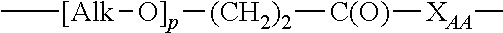

wherein
Alk designates C₂-C₄-alkylene,
X_{AA} is a dipeptide,
p is an integer from 0 to 50.
   R* is a reactive moiety selected from azide, alkynes, bisulfone, carbohydrazide, hydroxylamine, iodoacetamide, isothiocyanate, maleimide, phosphine, semihydrazide, succinimidyl ester and sulfonyl halide. Further, R* is or comprises optionally a bisulfone, optionally the bisulfone is or comprises

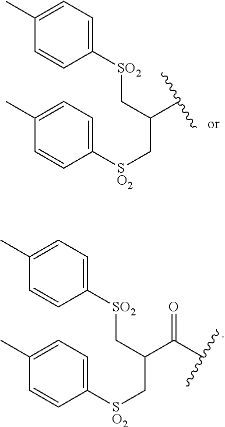 [SMILES: *C(CS(=O)(=O)c1ccc(C)cc1)CS(=O)(=O)c1ccc(C)cc1 ; *C(=O)C(CS(=O)(=O)c1ccc(C)cc1)CS(=O)(=O)c1ccc(C)cc1]

L₁ is —[CH₂]₀₋₁₂—C(O)NH—.
The drug moiety is selected from exatecan having the formula:

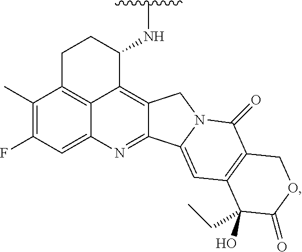 [SMILES: *N[C@H]1CCc2c(C)c(F)cc3nc4c(c1c23)Cn1c-4cc2c(c1=O)COC(=O)[C@]2(O)CC]

Dxd, Sn-38, monomethyl auristatin E (MMAE), and a pyrridinobenzodiazepine (PDD).

In one embodiment (CII), the disclosure provides a compound of formula (III), or salts, solvates, tautomers, isomers or mixtures thereof:

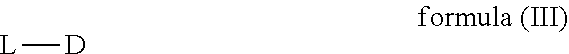

wherein in formula (III):
L is a linker; and
D comprises a drug moiety.
   The linker has the following formula:

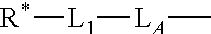

wherein L_{A} is a linker, L₁ is a linking moiety, and R* is a reactive moiety or a targeting agent. The linker L_{A} has the formula:

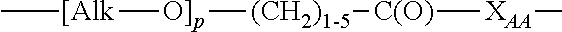

wherein
Alk designates C₂-C₄-alkylene,
X_{AA} is a dipeptide,
p is an integer from 0 to 50.
   R* is a reactive moiety selected from azide, alkynes, bisulfone, carbohydrazide, hydroxylamine, iodoacetamide, isothiocyanate, maleimide, phosphine, semihydrazide, succinimidyl ester and sulfonyl halide. Further, R* is or comprises optionally a bisulfone, optionally the bisulfone is or comprises

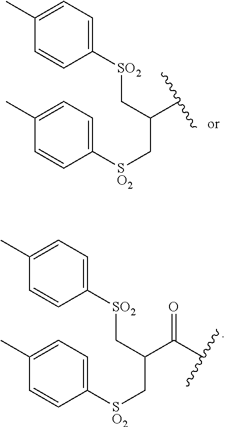 [SMILES: *C(CS(=O)(=O)c1ccc(C)cc1)CS(=O)(=O)c1ccc(C)cc1 ; *C(=O)C(CS(=O)(=O)c1ccc(C)cc1)CS(=O)(=O)c1ccc(C)cc1]

L₁ is —[CH₂]₀₋₁₂—C(O)NH—.
The drug moiety is selected from:

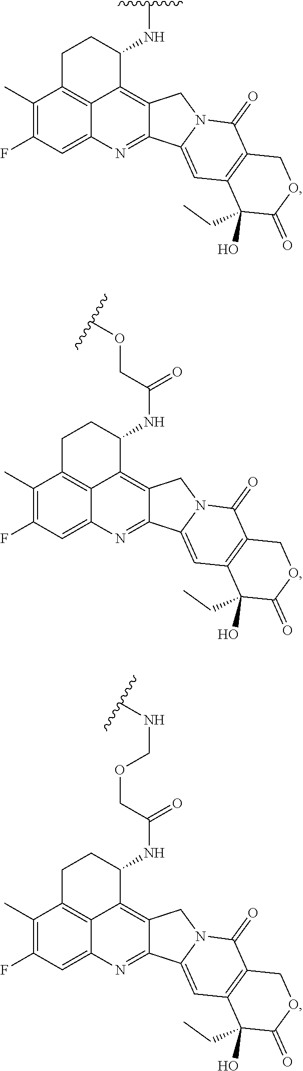 [SMILES: *N[C@H]1CCc2c(C)c(F)cc3nc4c(c1c23)Cn1c-4cc2c(c1=O)COC(=O)[C@]2(O)CC ; *OCC(=O)N[C@H]1CCc2c(C)c(F)cc3nc4c(c1c23)Cn1c-4cc2c(c1=O)COC(=O)[C@]2(O)CC ; *NCOCC(=O)N[C@H]1CCc2c(C)c(F)cc3nc4c(c1c23)Cn1c-4cc2c(c1=O)COC(=O)[C@]2(O)CC]

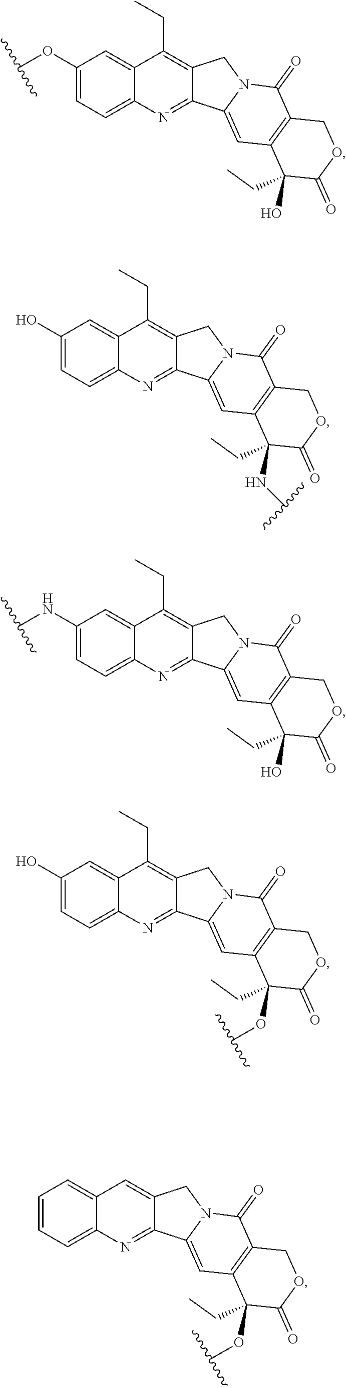 [SMILES: *Oc1ccc2nc3c(c(CC)c2c1)Cn1c-3cc2c(c1=O)COC(=O)[C@]2(O)CC ; *N[C@]1(CC)C(=O)OCc2c1cc1n(c2=O)Cc2c-1nc1ccc(O)cc1c2CC ; *Nc1ccc2nc3c(c(CC)c2c1)Cn1c-3cc2c(c1=O)COC(=O)[C@]2(O)CC ; *O[C@]1(CC)C(=O)OCc2c1cc1n(c2=O)Cc2c-1nc1ccc(O)cc1c2CC ; *O[C@]1(CC)C(=O)OCc2c1cc1n(c2=O)Cc2cc3ccccc3nc2-1]

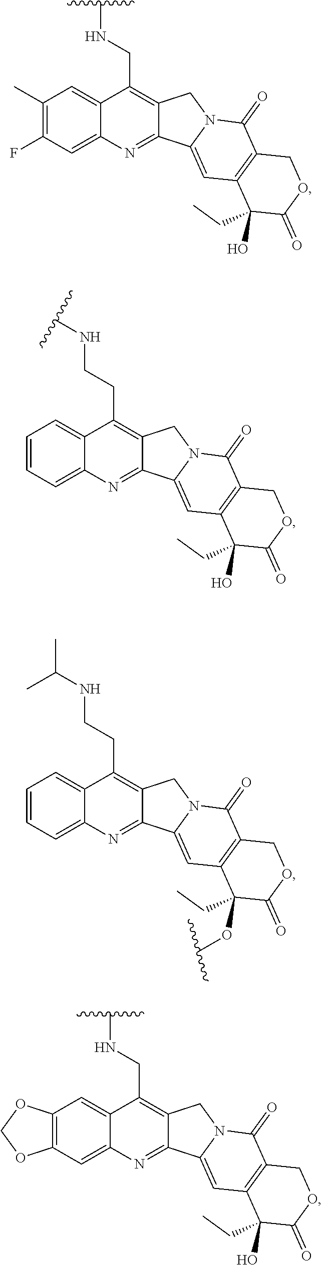 [SMILES: *NCc1c2c(nc3cc(F)c(C)cc13)-c1cc3c(c(=O)n1C2)COC(=O)[C@]3(O)CC ; *NCCc1c2c(nc3ccccc13)-c1cc3c(c(=O)n1C2)COC(=O)[C@]3(O)CC ; *O[C@]1(CC)C(=O)OCc2c1cc1n(c2=O)Cc2c-1nc1ccccc1c2CCNC(C)C ; *NCc1c2c(nc3cc4c(cc13)OCO4)-c1cc3c(c(=O)n1C2)COC(=O)[C@]3(O)CC]

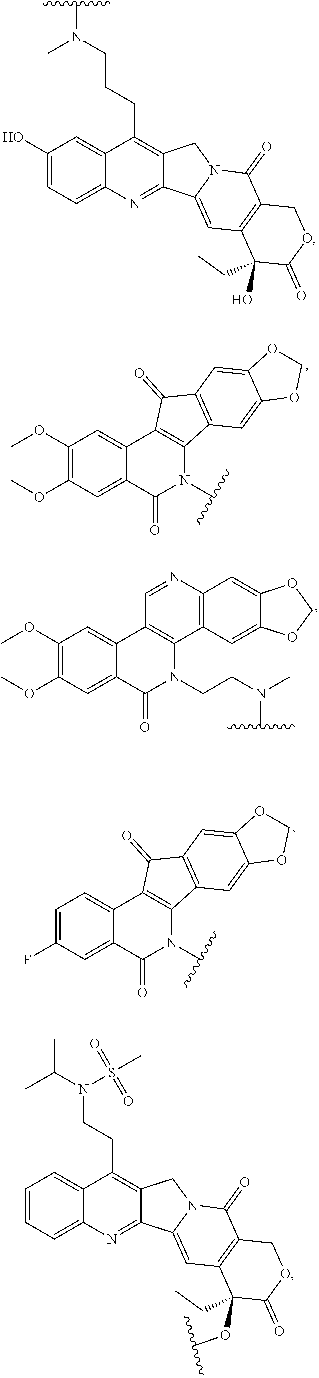 [SMILES: *N(C)CCCc1c2c(nc3ccc(O)cc13)-c1cc3c(c(=O)n1C2)COC(=O)[C@]3(O)CC ; *n1c2c(c3cc(OC)c(OC)cc3c1=O)C(=O)c1cc3c(cc1-2)OCO3 ; *N(C)CCn1c(=O)c2cc(OC)c(OC)cc2c2cnc3cc4c(cc3c21)OCO4 ; *n1c2c(c3ccc(F)cc3c1=O)C(=O)c1cc3c(cc1-2)OCO3 ; *O[C@]1(CC)C(=O)OCc2c1cc1n(c2=O)Cc2c-1nc1ccccc1c2CCN(C(C)C)S(C)(=O)=O]

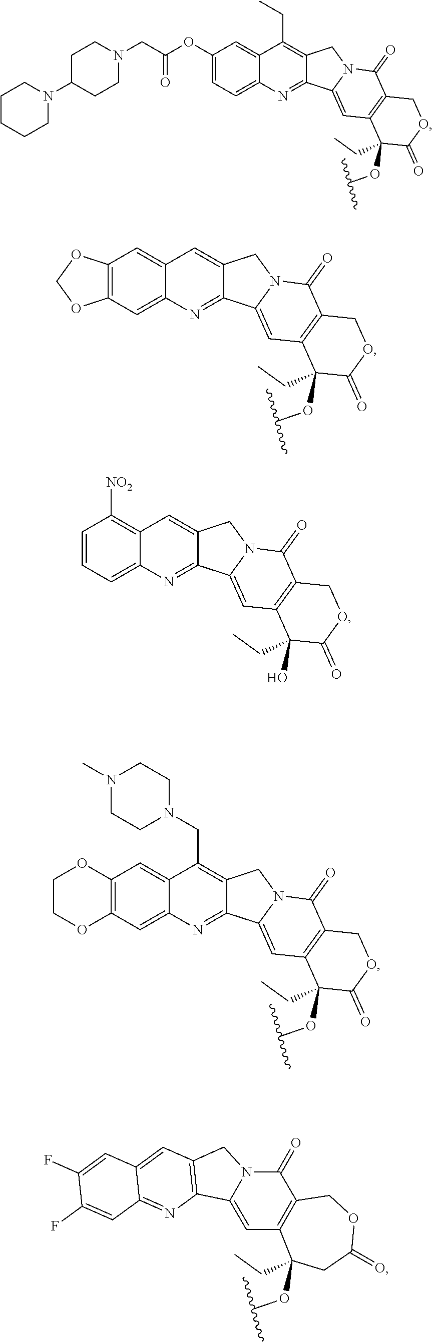 [SMILES: *O[C@]1(CC)C(=O)OCc2c1cc1n(c2=O)Cc2c-1nc1ccc(OC(=O)CN3CCC(N4CCCCC4)CC3)cc1c2CC ; *O[C@]1(CC)C(=O)OCc2c1cc1n(c2=O)Cc2cc3cc4c(cc3nc2-1)OCO4 ; CC[C@@]1(O)C(=O)OCc2c1cc1n(c2=O)Cc2cc3c([N+](=O)[O-])cccc3nc2-1 ; *O[C@]1(CC)C(=O)OCc2c1cc1n(c2=O)Cc2c-1nc1cc3c(cc1c2CN1CCN(C)CC1)OCCO3 ; *O[C@]1(CC)CC(=O)OCc2c1cc1n(c2=O)Cc2cc3cc(F)c(F)cc3nc2-1]

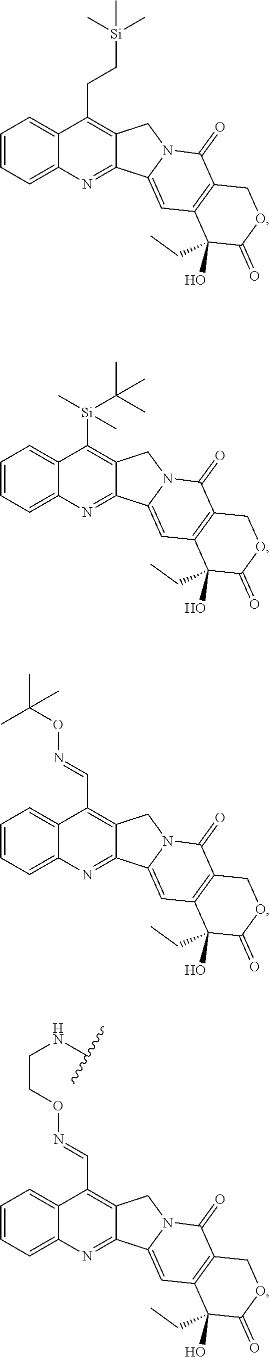 [SMILES: CC[C@@]1(O)C(=O)OCc2c1cc1n(c2=O)Cc2c-1nc1ccccc1c2CC[Si](C)(C)C ; CC[C@@]1(O)C(=O)OCc2c1cc1n(c2=O)Cc2c-1nc1ccccc1c2[Si](C)(C)C(C)(C)C ; CC[C@@]1(O)C(=O)OCc2c1cc1n(c2=O)Cc2c-1nc1ccccc1c2/C=N/OC(C)(C)C ; *NCCO/N=C/c1c2c(nc3ccccc13)-c1cc3c(c(=O)n1C2)COC(=O)[C@]3(O)CC]

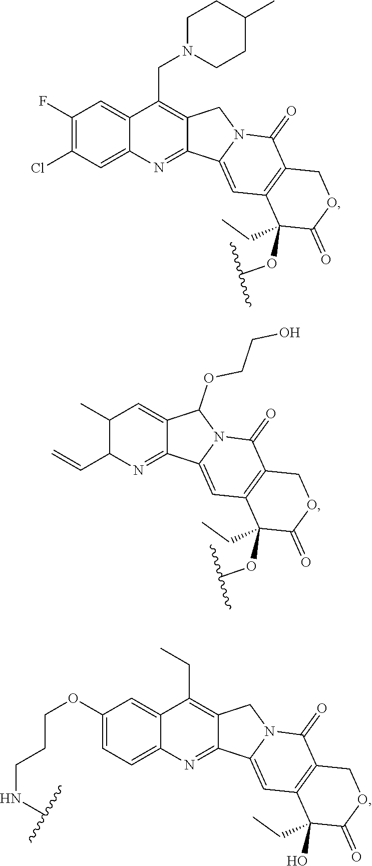 [SMILES: *O[C@]1(CC)C(=O)OCc2c1cc1n(c2=O)Cc2c-1nc1cc(Cl)c(F)cc1c2CN1CCC(C)CC1 ; *O[C@]1(CC)C(=O)OCc2c1cc1n(c2=O)C(OCCO)C2=CC(C)C(C=C)N=C21 ; *NCCCOc1ccc2nc3c(c(CC)c2c1)Cn1c-3cc2c(c1=O)COC(=O)[C@]2(O)CC]

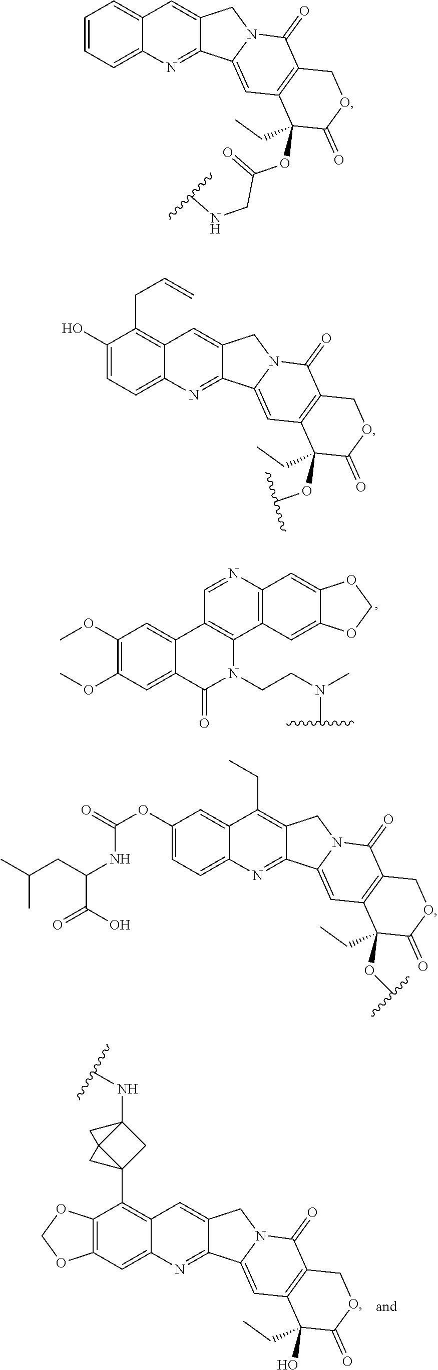

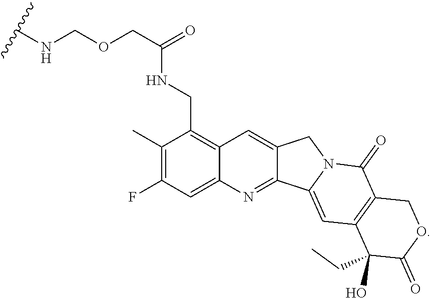 [SMILES: *NCOCC(=O)NCc1c(C)c(F)cc2nc3c(cc12)Cn1c-3cc2c(c1=O)COC(=O)[C@]2(O)CC]

In one embodiment (CIIa), the disclosure provides a compound of formula (III), or salts, solvates, tautomers, isomers or mixtures thereof:

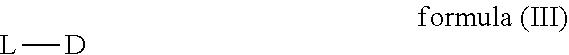

wherein in formula (III):
L is a linker; and
D comprises a drug moiety.
   The linker has the following formula:

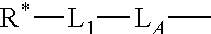

wherein L_{A} is a linker, L₁ is a linking moiety, and R* is a reactive moiety or a targeting agent. The linker L_{A} has the formula:

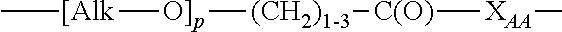

wherein
Alk designates C₂-C₄-alkylene,
X_{AA} is a dipeptide,
p is an integer from 0 to 50.
   R* is a reactive moiety selected from azide, alkynes, bisulfone, carbohydrazide, hydroxylamine, iodoacetamide, isothiocyanate, maleimide, phosphine, semihydrazide, succinimidyl ester and sulfonyl halide. Further, R* is or comprises optionally a bisulfone, optionally the bisulfone is or comprises

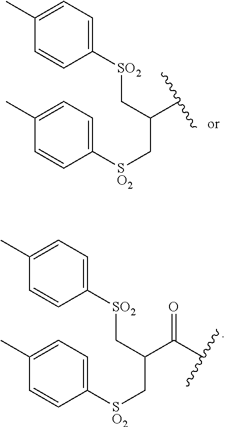 [SMILES: *C(CS(=O)(=O)c1ccc(C)cc1)CS(=O)(=O)c1ccc(C)cc1 ; *C(=O)C(CS(=O)(=O)c1ccc(C)cc1)CS(=O)(=O)c1ccc(C)cc1]

L₁ is —[CH₂]₀₋₁₂—C(O)NH—.
The drug moiety is selected from:

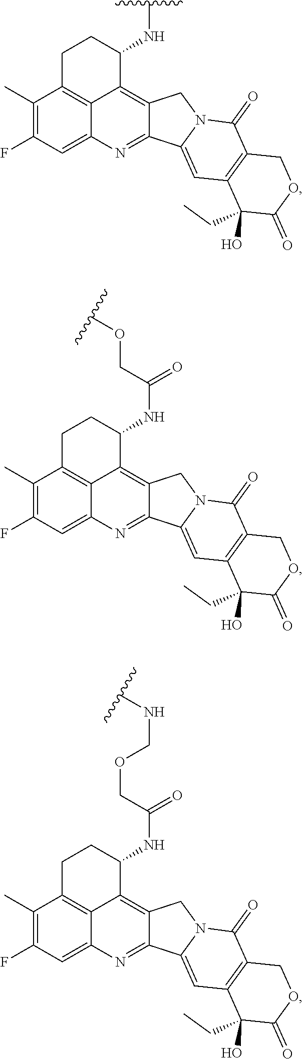 [SMILES: *N[C@H]1CCc2c(C)c(F)cc3nc4c(c1c23)Cn1c-4cc2c(c1=O)COC(=O)[C@]2(O)CC ; *OCC(=O)N[C@H]1CCc2c(C)c(F)cc3nc4c(c1c23)Cn1c-4cc2c(c1=O)COC(=O)[C@]2(O)CC ; *NCOCC(=O)N[C@H]1CCc2c(C)c(F)cc3nc4c(c1c23)Cn1c-4cc2c(c1=O)COC(=O)[C@]2(O)CC]

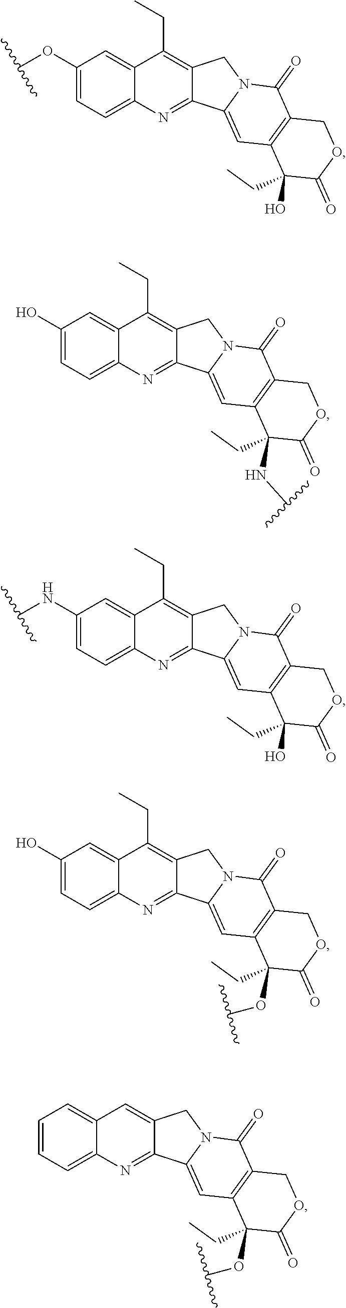 [SMILES: *Oc1ccc2nc3c(c(CC)c2c1)Cn1c-3cc2c(c1=O)COC(=O)[C@]2(O)CC ; *N[C@]1(CC)C(=O)OCc2c1cc1n(c2=O)Cc2c-1nc1ccc(O)cc1c2CC ; *Nc1ccc2nc3c(c(CC)c2c1)Cn1c-3cc2c(c1=O)COC(=O)[C@]2(O)CC ; *O[C@]1(CC)C(=O)OCc2c1cc1n(c2=O)Cc2c-1nc1ccc(O)cc1c2CC ; *O[C@]1(CC)C(=O)OCc2c1cc1n(c2=O)Cc2cc3ccccc3nc2-1]

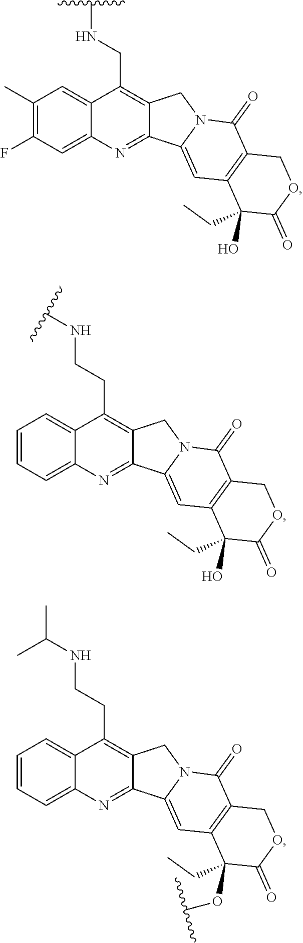 [SMILES: *NCc1c2c(nc3cc(F)c(C)cc13)-c1cc3c(c(=O)n1C2)COC(=O)[C@]3(O)CC ; *NCCc1c2c(nc3ccccc13)-c1cc3c(c(=O)n1C2)COC(=O)[C@]3(O)CC ; *O[C@]1(CC)C(=O)OCc2c1cc1n(c2=O)Cc2c-1nc1ccccc1c2CCNC(C)C]

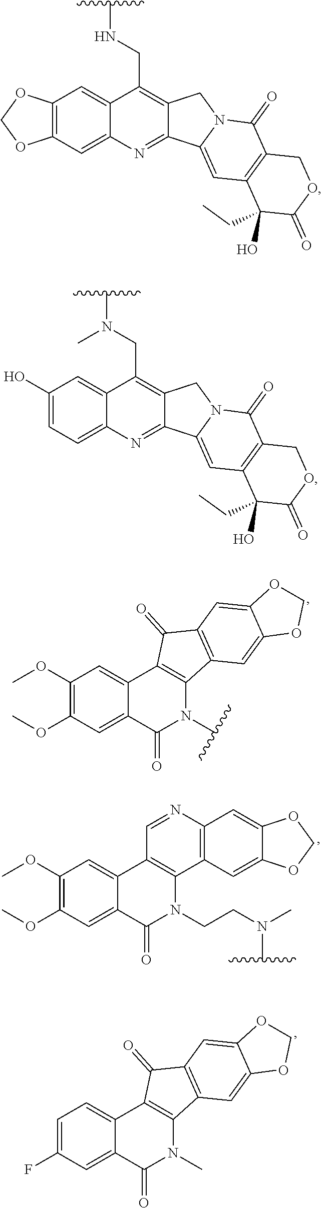 [SMILES: *NCc1c2c(nc3cc4c(cc13)OCO4)-c1cc3c(c(=O)n1C2)COC(=O)[C@]3(O)CC ; *N(C)Cc1c2c(nc3ccc(O)cc13)-c1cc3c(c(=O)n1C2)COC(=O)[C@]3(O)CC ; *n1c2c(c3cc(OC)c(OC)cc3c1=O)C(=O)c1cc3c(cc1-2)OCO3 ; *N(C)CCn1c(=O)c2cc(OC)c(OC)cc2c2cnc3cc4c(cc3c21)OCO4 ; Cn1c2c(c3ccc(F)cc3c1=O)C(=O)c1cc3c(cc1-2)OCO3]

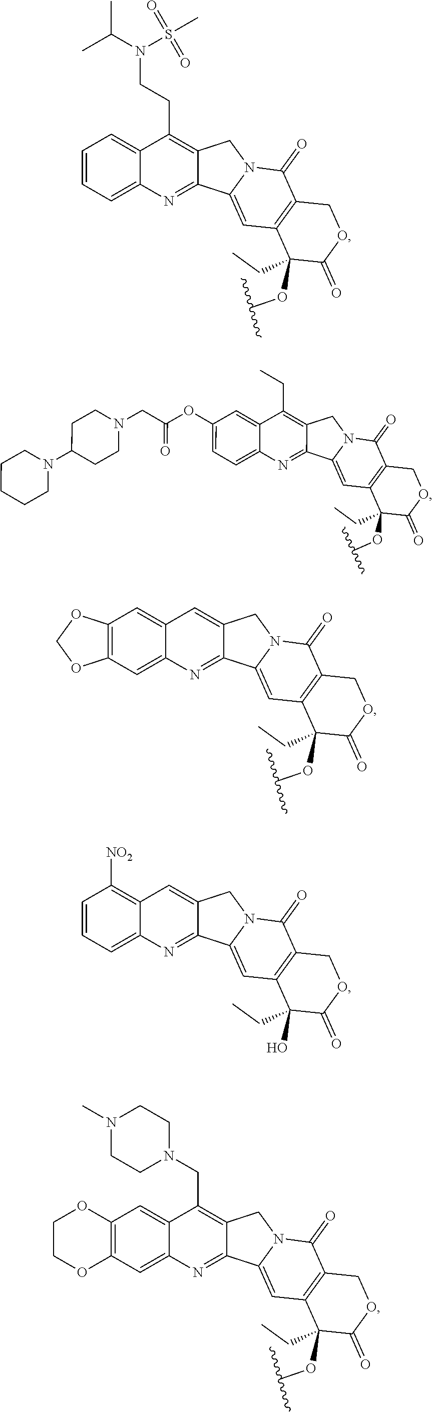 [SMILES: *O[C@]1(CC)C(=O)OCc2c1cc1n(c2=O)Cc2c-1nc1ccccc1c2CCN(C(C)C)S(C)(=O)=O ; *O[C@]1(CC)C(=O)OCc2c1cc1n(c2=O)Cc2c-1nc1ccc(OC(=O)CN3CCC(N4CCCCC4)CC3)cc1c2CC ; *O[C@]1(CC)C(=O)OCc2c1cc1n(c2=O)Cc2cc3cc4c(cc3nc2-1)OCO4 ; CC[C@@]1(O)C(=O)OCc2c1cc1n(c2=O)Cc2cc3c([N+](=O)[O-])cccc3nc2-1 ; *O[C@]1(CC)C(=O)OCc2c1cc1n(c2=O)Cc2c-1nc1cc3c(cc1c2CN1CCN(C)CC1)OCCO3]

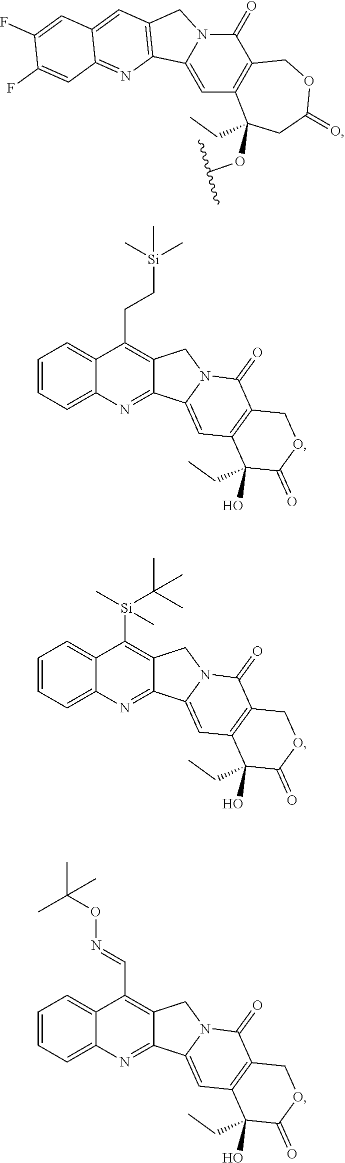 [SMILES: *O[C@]1(CC)CC(=O)OCc2c1cc1n(c2=O)Cc2cc3cc(F)c(F)cc3nc2-1 ; CC[C@@]1(O)C(=O)OCc2c1cc1n(c2=O)Cc2c-1nc1ccccc1c2CC[Si](C)(C)C ; CC[C@@]1(O)C(=O)OCc2c1cc1n(c2=O)Cc2c-1nc1ccccc1c2[Si](C)(C)C(C)(C)C ; CC[C@@]1(O)C(=O)OCc2c1cc1n(c2=O)Cc2c-1nc1ccccc1c2/C=N/OC(C)(C)C]

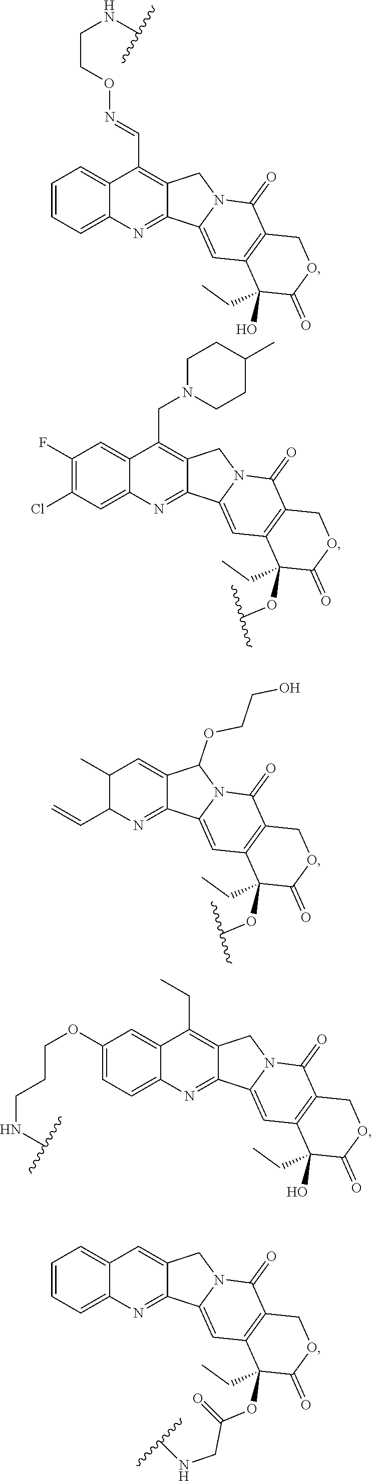

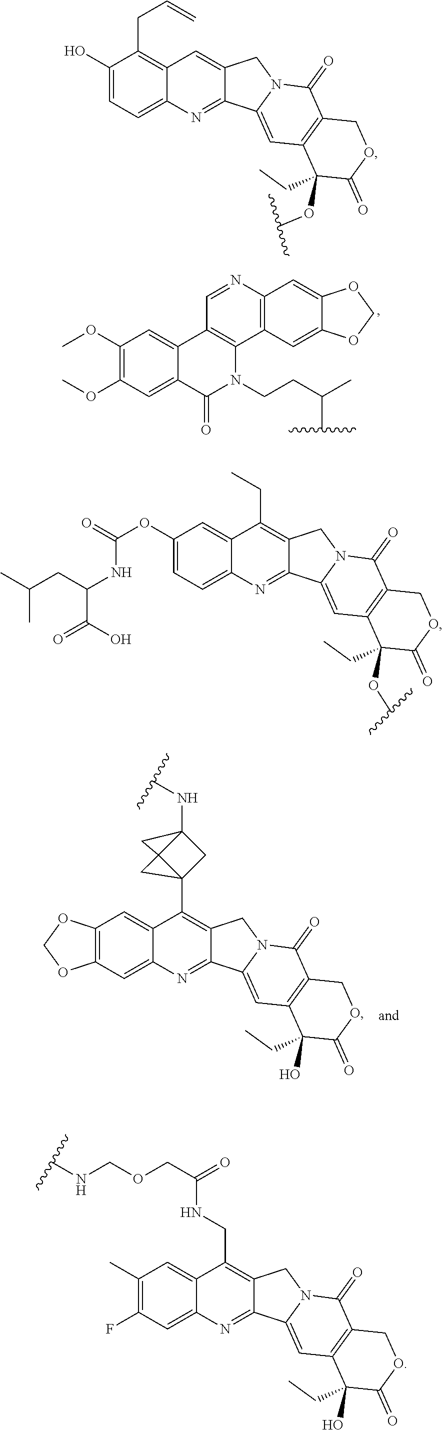 [SMILES: *O[C@]1(CC)C(=O)OCc2c1cc1n(c2=O)Cc2cc3c(CC=C)c(O)ccc3nc2-1 ; *C(C)CCn1c(=O)c2cc(OC)c(OC)cc2c2cnc3cc4c(cc3c21)OCO4 ; *O[C@]1(CC)C(=O)OCc2c1cc1n(c2=O)Cc2c-1nc1ccc(OC(=O)NC(CC(C)C)C(=O)O)cc1c2CC ; *NC12CC3(c4c5c(nc6cc7c(cc46)OCO7)-c4cc6c(c(=O)n4C5)COC(=O)[C@]6(O)CC)CC13C2 ; *NCOCC(=O)NCc1c2c(nc3cc(F)c(C)cc13)-c1cc3c(c(=O)n1C2)COC(=O)[C@]3(O)CC]

In one embodiment (CIIb), the disclosure provides a compound of formula (III), or salts, solvates, tautomers, isomers or mixtures thereof:

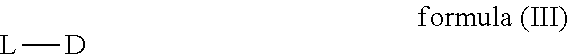

wherein in formula (III):
L is a linker; and
D comprises a drug moiety.
   The linker has the following formula:

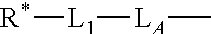

wherein L_{A} is a linker, L₁ is a linking moiety, and R* is a reactive moiety or a targeting agent. The linker L_{A} has the formula:

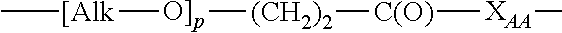

wherein
Alk designates C₂-C₄-alkylene,
X_{AA} is a dipeptide,
p is an integer from 0 to 50.
   R* is a reactive moiety selected from azide, alkynes, bisulfone, carbohydrazide, hydroxylamine, iodoacetamide, isothiocyanate, maleimide, phosphine, semihydrazide, succinimidyl ester and sulfonyl halide. Further, R* is or comprises optionally a bisulfone, optionally the bisulfone is or comprises

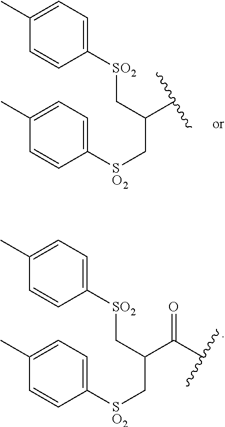 [SMILES: *C(CS(=O)(=O)c1ccc(C)cc1)CS(=O)(=O)c1ccc(C)cc1 ; *C(=O)C(CS(=O)(=O)c1ccc(C)cc1)CS(=O)(=O)c1ccc(C)cc1]

L₁ is —[CH₂]₀₋₁₂—C(O)NH—.
The drug moiety is selected from:

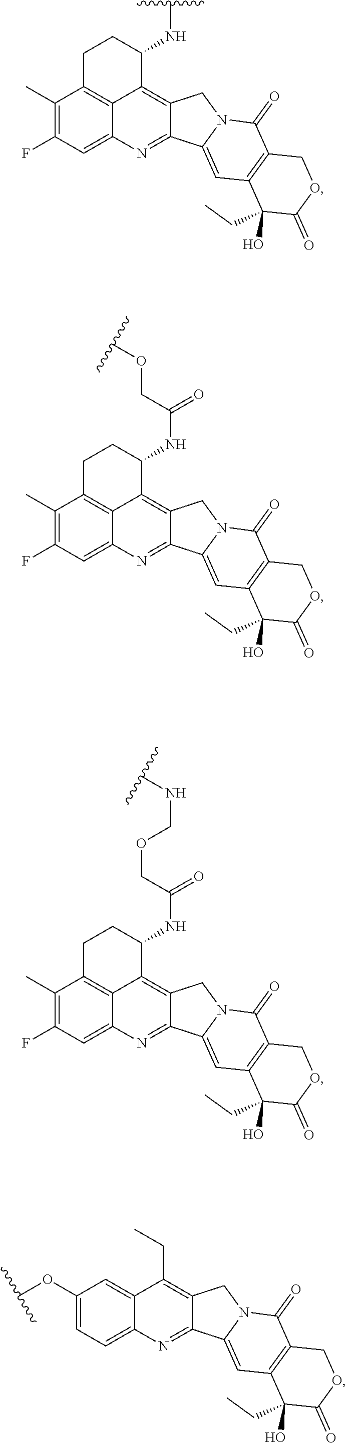 [SMILES: *N[C@H]1CCc2c(C)c(F)cc3nc4c(c1c23)Cn1c-4cc2c(c1=O)COC(=O)[C@]2(O)CC ; *OCC(=O)N[C@H]1CCc2c(C)c(F)cc3nc4c(c1c23)Cn1c-4cc2c(c1=O)COC(=O)[C@]2(O)CC ; *NCOCC(=O)N[C@H]1CCc2c(C)c(F)cc3nc4c(c1c23)Cn1c-4cc2c(c1=O)COC(=O)[C@]2(O)CC ; *Oc1ccc2nc3c(c(CC)c2c1)Cn1c-3cc2c(c1=O)COC(=O)[C@]2(O)CC]

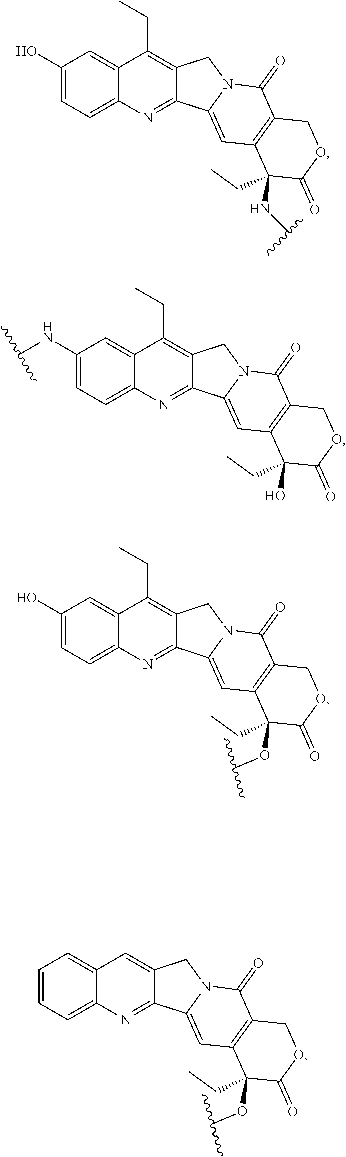 [SMILES: *N[C@]1(CC)C(=O)OCc2c1cc1n(c2=O)Cc2c-1nc1ccc(O)cc1c2CC ; *Nc1ccc2nc3c(c(CC)c2c1)Cn1c-3cc2c(c1=O)COC(=O)[C@]2(O)CC ; *O[C@]1(CC)C(=O)OCc2c1cc1n(c2=O)Cc2c-1nc1ccc(O)cc1c2CC ; *O[C@]1(CC)C(=O)OCc2c1cc1n(c2=O)Cc2cc3ccccc3nc2-1]

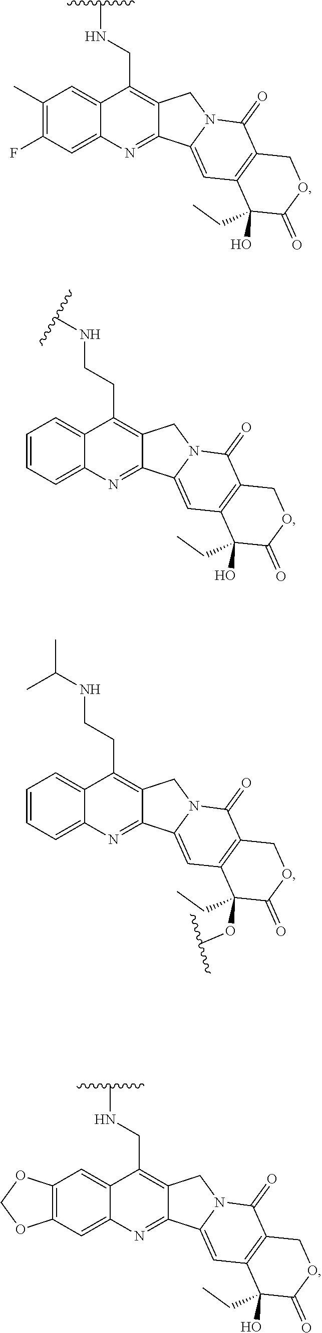 [SMILES: *NCc1c2c(nc3cc(F)c(C)cc13)-c1cc3c(c(=O)n1C2)COC(=O)[C@]3(O)CC ; *NCCc1c2c(nc3ccccc13)-c1cc3c(c(=O)n1C2)COC(=O)[C@]3(O)CC ; *O[C@]1(CC)C(=O)OCc2c1cc1n(c2=O)Cc2c-1nc1ccccc1c2CCNC(C)C ; *NCc1c2c(nc3cc4c(cc13)OCO4)-c1cc3c(c(=O)n1C2)COC(=O)[C@]3(O)CC]

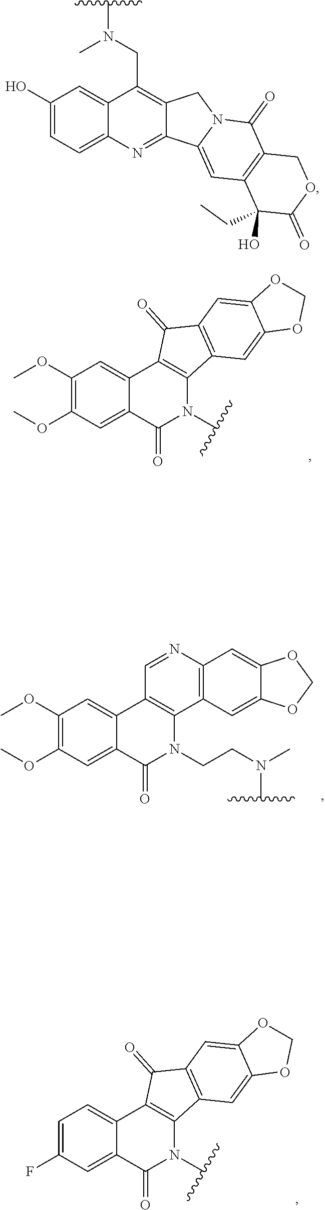

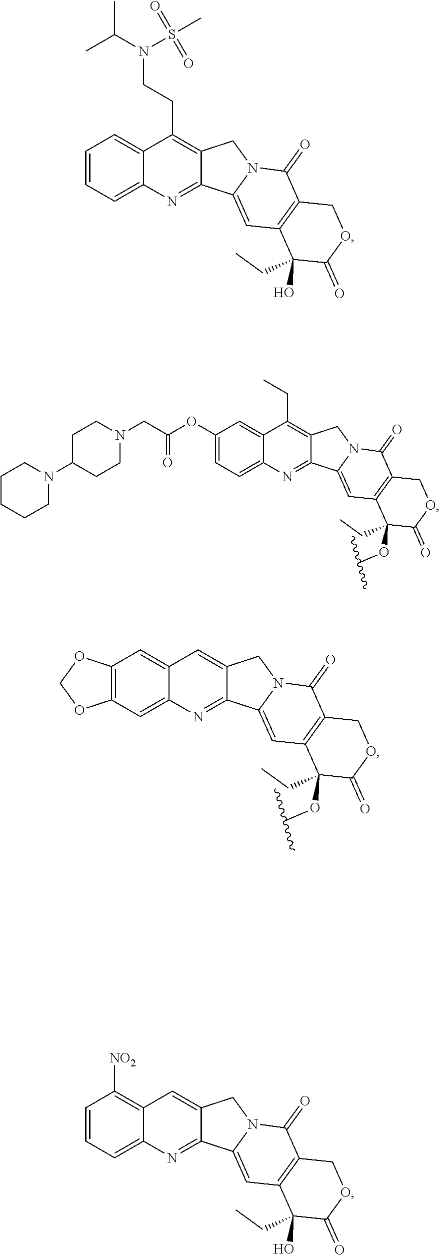 [SMILES: CC[C@@]1(O)C(=O)OCc2c1cc1n(c2=O)Cc2c-1nc1ccccc1c2CCN(C(C)C)S(C)(=O)=O ; *O[C@]1(CC)C(=O)OCc2c1cc1n(c2=O)Cc2c-1nc1ccc(OC(=O)CN3CCC(N4CCCCC4)CC3)cc1c2CC ; *O[C@]1(CC)C(=O)OCc2c1cc1n(c2=O)Cc2cc3cc4c(cc3nc2-1)OCO4 ; CC[C@@]1(O)C(=O)OCc2c1cc1n(c2=O)Cc2cc3c([N+](=O)[O-])cccc3nc2-1]

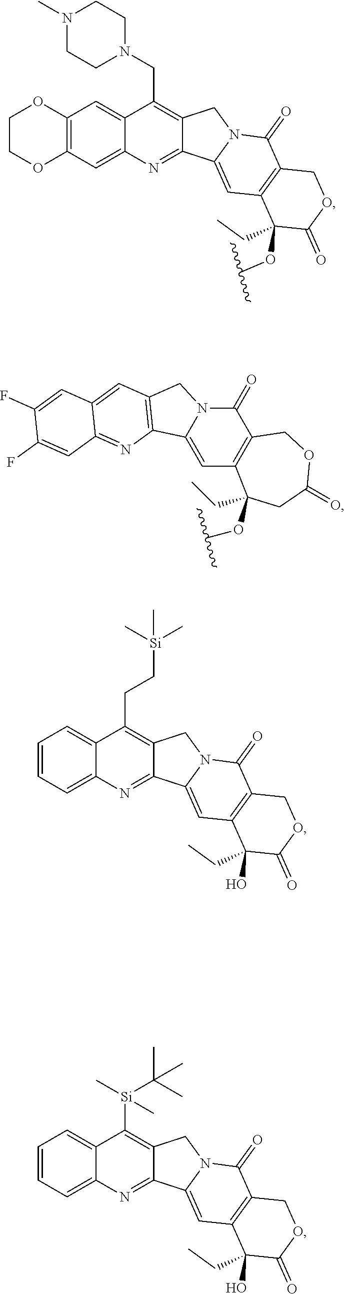 [SMILES: *O[C@]1(CC)C(=O)OCc2c1cc1n(c2=O)Cc2c-1nc1cc3c(cc1c2CN1CCN(C)CC1)OCCO3 ; *O[C@]1(CC)CC(=O)OCc2c1cc1n(c2=O)Cc2cc3cc(F)c(F)cc3nc2-1 ; CC[C@@]1(O)C(=O)OCc2c1cc1n(c2=O)Cc2c-1nc1ccccc1c2CC[Si](C)(C)C ; CC[C@@]1(O)C(=O)OCc2c1cc1n(c2=O)Cc2c-1nc1ccccc1c2[Si](C)(C)C(C)(C)C]

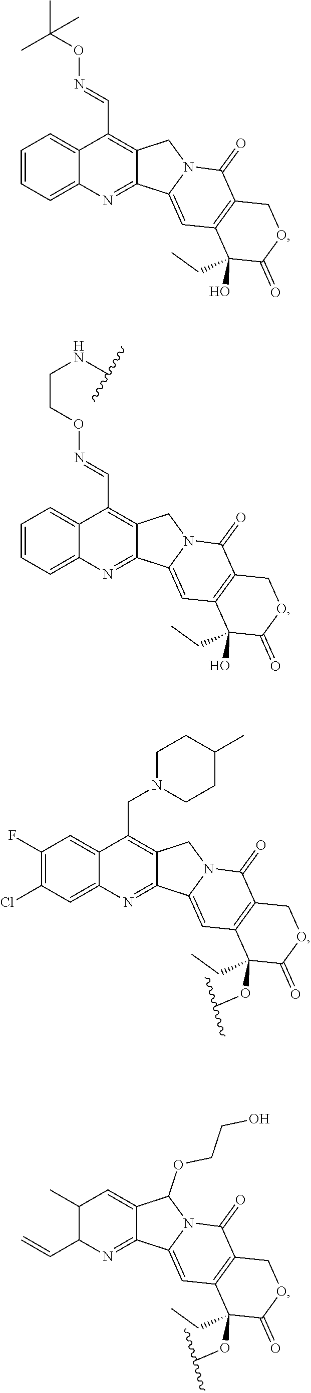 [SMILES: CC[C@@]1(O)C(=O)OCc2c1cc1n(c2=O)Cc2c-1nc1ccccc1c2/C=N/OC(C)(C)C ; *NCCO/N=C/c1c2c(nc3ccccc13)-c1cc3c(c(=O)n1C2)COC(=O)[C@]3(O)CC ; *O[C@]1(CC)C(=O)OCc2c1cc1n(c2=O)Cc2c-1nc1cc(Cl)c(F)cc1c2CN1CCC(C)CC1 ; *O[C@]1(CC)C(=O)OCc2c1cc1n(c2=O)C(OCCO)C2=CC(C)C(C=C)N=C21]

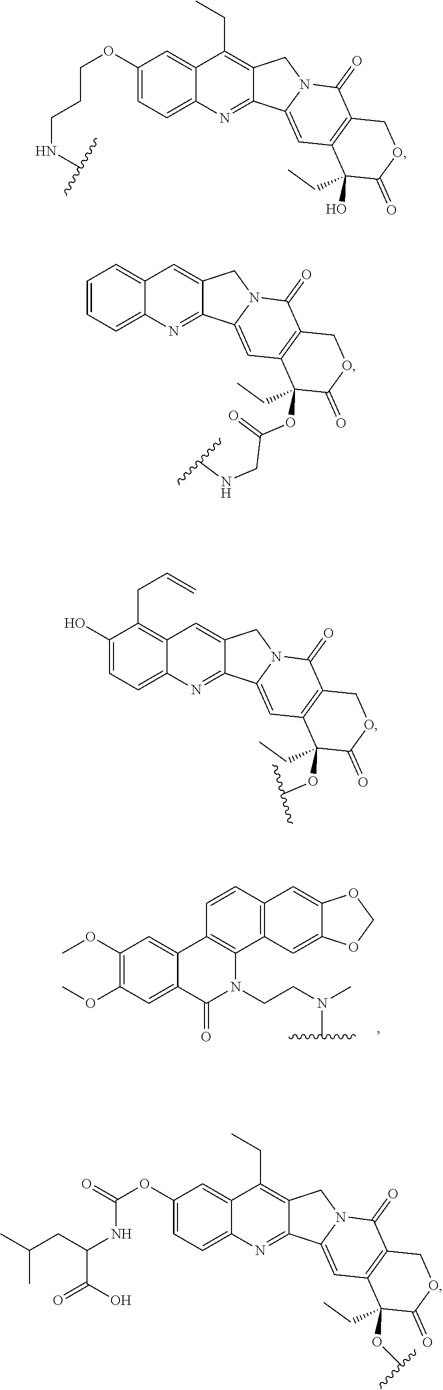 [SMILES: *NCCCOc1ccc2nc3c(c(CC)c2c1)Cn1c-3cc2c(c1=O)COC(=O)[C@]2(O)CC ; *NCC(=O)O[C@]1(CC)C(=O)OCc2c1cc1n(c2=O)Cc2cc3ccccc3nc2-1 ; *O[C@]1(CC)C(=O)OCc2c1cc1n(c2=O)Cc2cc3c(CC=C)c(O)ccc3nc2-1 ; *N(C)CCn1c(=O)c2cc(OC)c(OC)cc2c2ccc3cc4c(cc3c21)OCO4 ; *O[C@]1(CC)C(=O)OCc2c1cc1n(c2=O)Cc2c-1nc1ccc(OC(=O)NC(CC(C)C)C(=O)O)cc1c2CC]

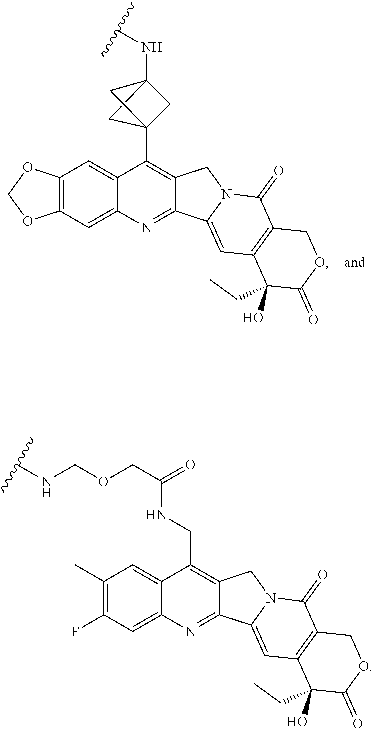 [SMILES: *NC12CC(c3c4c(nc5cc6c(cc35)OCO6)-c3cc5c(c(=O)n3C4)COC(=O)[C@]5(O)CC)(C1)C2 ; "and" ; *NCOCC(=O)NCc1c2c(nc3cc(F)c(C)cc13)-c1cc3c(c(=O)n1C2)COC(=O)[C@]3(O)CC]

In one embodiment (CIII), the disclosure provides a compound of formula (III), or salts, solvates, tautomers, isomers or mixtures thereof:

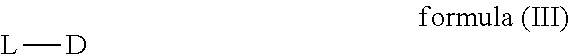

wherein in formula (III):
L is a linker; and
D comprises a drug moiety.
   The linker has the following formula:

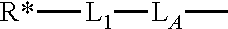

wherein L_{A} is a linker, L₁ is a linking moiety, and R* is a reactive moiety or a targeting agent. The linker L_{A} has the formula:

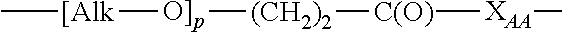

wherein
X_{AA} is a nonpolar dipeptide,
p is an integer from 5 to 10, e.g. 5, 6, 7, 8, 9, or 10.
   R* is a reactive moiety selected from azide, alkynes, bisulfone, carbohydrazide, hydroxylamine, iodoacetamide, isothiocyanate, maleimide, phosphine, semihydrazide, succinimidyl ester and sulfonyl halide. Further, R* is or comprises optionally a bisulfone, optionally the bisulfone is or comprises

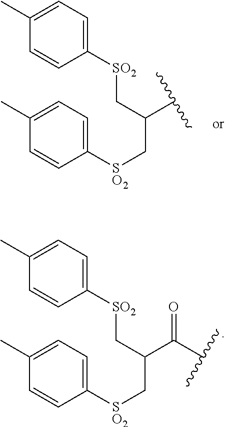 [SMILES: *C(CS(=O)(=O)c1ccc(C)cc1)CS(=O)(=O)c1ccc(C)cc1 ; *C(=O)C(CS(=O)(=O)c1ccc(C)cc1)CS(=O)(=O)c1ccc(C)cc1]

L₁ is —[CH₂]₀₋₁₂—C(O)NH—.
The drug moiety is selected from exatecan having the formula:

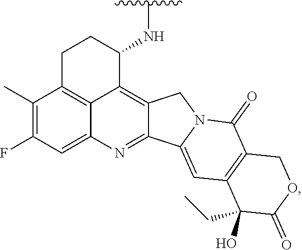 [SMILES: *N[C@H]1CCc2c(C)c(F)cc3nc4c(c1c23)Cn1c-4cc2c(c1=O)COC(=O)[C@]2(O)CC]

Dxd, Sn-38, monomethyl auristatin E (MMAE), and a pyrridinobenzodiazepine (PDD).

In one embodiment (CIV), the disclosure provides a compound of formula (III), or salts, solvates, tautomers, isomers or mixtures thereof:

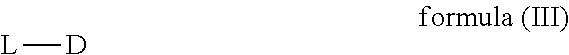

wherein in formula (III):
L is a linker; and
D comprises a drug moiety.
   The linker has the following formula:

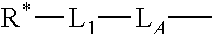

wherein L_{A} is a linker, L₁ is a linking moiety, and R* is a reactive moiety or a targeting agent. The linker L_{A} has the formula:

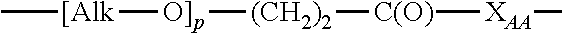

wherein
Alk designates C₂-C₄-alkylene,
X_{AA} is a dipeptide,
p is an integer from 0 to 50.
   R* is maleimide.
   L₁ is —[CH₂]₁₋₃—C(O)NH—.
   The drug moiety is selected from exatecan having the formula:

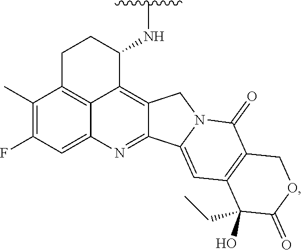 [SMILES: *N[C@H]1CCc2c(C)c(F)cc3nc4c(c1c23)Cn1c-4cc2c(c1=O)COC(=O)[C@]2(O)CC]

Dxd, Sn-38, monomethyl auristatin E (MMAE), and a pyrridinobenzodiazepine (PDD).

In one embodiment (CV), the disclosure provides a compound of formula (III), or salts, solvates, tautomers, isomers or mixtures thereof:

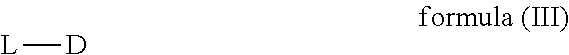

wherein in formula (III):
L is a linker; and
D comprises a drug moiety.
   The linker has the following formula:

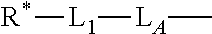

wherein L_{A} is a linker, L₁ is a linking moiety, and R* is a reactive moiety or a targeting agent. The linker L_{A} has the formula:

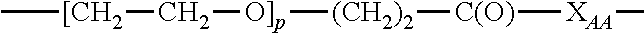

wherein
X_{AA} is a nonpolar dipeptide,
p is an integer from 5 to 10, e.g. 5, 6, 7, 8, 9, or 10.
   R* is maleimide
   L₁ is —[CH₂]₁₋₃—C(O)NH—.
   The drug moiety is selected from exatecan having the formula:

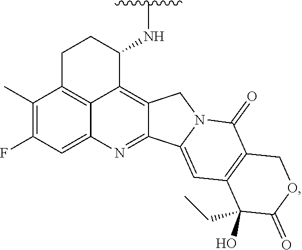 [SMILES: *N[C@H]1CCc2c(C)c(F)cc3nc4c(c1c23)Cn1c-4cc2c(c1=O)COC(=O)[C@]2(O)CC]

Dxd, Sn-38, monomethyl auristatin E (MMAE), and a pyrridinobenzodiazepine (PDD).

In one embodiment (CVI), the disclosure provides a compound of formula (III), or salts, solvates, tautomers, isomers or mixtures thereof:

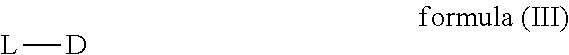

wherein in formula (III):
L is a linker; and
D comprises a drug moiety.
   The linker has the following formula:

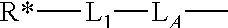

wherein L_{A} is a linker, L₁ is a linking moiety, and R* is a reactive moiety or a targeting agent. The linker L_{A} has the formula:

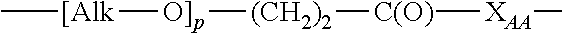

wherein
Alk designates C₂-C₄-alkylene,
X_{AA} is Val-Ala,
p is 7 or 8.
   R* is a reactive moiety selected from azide, alkynes, bisulfone, carbohydrazide, hydroxylamine, iodoacetamide, isothiocyanate, maleimide, phosphine, semihydrazide, succinimidyl ester and sulfonyl halide. Further, R* is or comprises optionally a bisulfone, optionally the bisulfone is or comprises

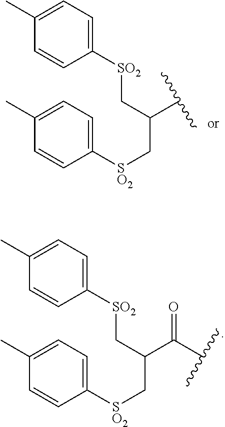 [SMILES: *C(CS(=O)(=O)c1ccc(C)cc1)CS(=O)(=O)c1ccc(C)cc1 ; *C(=O)C(CS(=O)(=O)c1ccc(C)cc1)CS(=O)(=O)c1ccc(C)cc1]

L₁ is —[CH₂]₀₋₁₂—C(O)NH—.

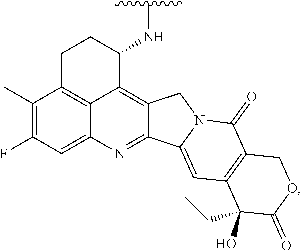 [SMILES: *N[C@H]1CCc2c(C)c(F)cc3nc4c(c1c23)Cn1c-4cc2c(c1=O)COC(=O)[C@]2(O)CC]

Dxd, Sn-38, monomethyl auristatin E (MMAE), and a pyrridinobenzodiazepine (PDD).

In one embodiment (CVII), the disclosure provides a compound of formula (III), or salts, solvates, tautomers, isomers or mixtures thereof:

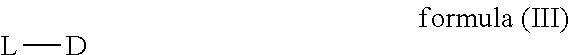

wherein in formula (III):
L is a linker; and
D comprises a drug moiety.
   The linker has the following formula:

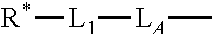

wherein L_{A} is a linker, L₁ is a linking moiety, and R* is a reactive moiety or a targeting agent. The linker L_{A} has the formula:

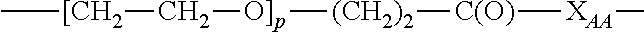

wherein
X_{AA} is Val-Ala,
p is 7 or 8.
   R* is a reactive moiety selected from azide, alkynes, bisulfone, carbohydrazide, hydroxylamine, iodoacetamide, isothiocyanate, maleimide, phosphine, semihydrazide, succinimidyl ester and sulfonyl halide. Further, R* is or comprises optionally a bisulfone, optionally the bisulfone is or comprises

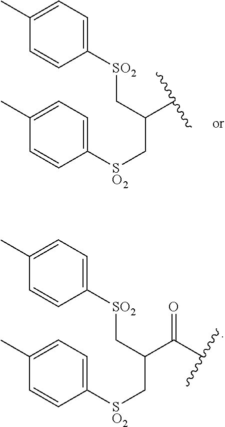 [SMILES: *C(CS(=O)(=O)c1ccc(C)cc1)CS(=O)(=O)c1ccc(C)cc1 ; *C(=O)C(CS(=O)(=O)c1ccc(C)cc1)CS(=O)(=O)c1ccc(C)cc1]

L₁ is —[CH₂]₀₋₁₂—C(O)NH—.

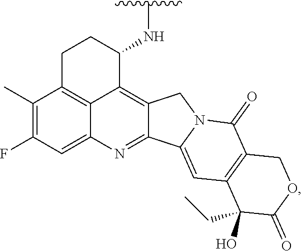 [SMILES: *N[C@H]1CCc2c(C)c(F)cc3nc4c(c1c23)Cn1c-4cc2c(c1=O)COC(=O)[C@]2(O)CC]

Dxd, Sn-38, monomethyl auristatin E (MMAE), and a pyrridinobenzodiazepine (PDD).

In one embodiment (CVIII), the disclosure provides a compound of formula (III), or salts, solvates, tautomers, isomers or mixtures thereof:

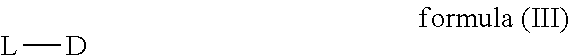

wherein in formula (III):
L is a linker; and
D comprises a drug moiety.
   The linker has the following formula:

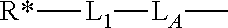

wherein L_{A} is a linker, L₁ is a linking moiety, and R* is a reactive moiety or a targeting agent. The linker L_{A} has the formula:

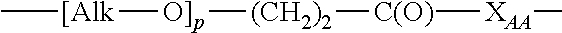

wherein
Alk designates C₂-C₄-alkylene,
X_{AA} is Val-Ala,
p is 7 or 8.
   R* is maleimide.
   L₁ is —[CH₂]₁₋₃—C(O)NH—.
   The drug moiety is selected from exatecan having the formula:

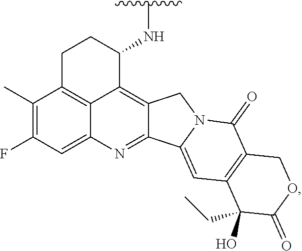 [SMILES: *N[C@H]1CCc2c(C)c(F)cc3nc4c(c1c23)Cn1c-4cc2c(c1=O)COC(=O)[C@]2(O)CC]

Dxd, Sn-38, monomethyl auristatin E (MMAE), and a pyrridinobenzodiazepine (PDD).

In one embodiment (CIX), the disclosure provides a compound of formula (III), or salts, solvates, tautomers, isomers or mixtures thereof:

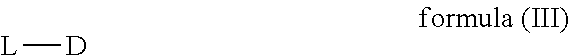

wherein in formula (III):
L is a linker; and
D comprises a drug moiety.
   The linker has the following formula:

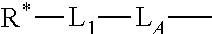

wherein L_{A} is a linker, L₁ is a linking moiety, and R* is a reactive moiety or a targeting agent. The linker L_{A} has the formula:

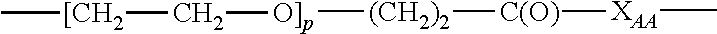

wherein
X_{AA} is Val-Ala,
p is 7 or 8.
   R* is maleimide.
   L₁ is —[CH₂]₁₋₃—C(O)NH—.
   The drug moiety is selected from exatecan having the formula:

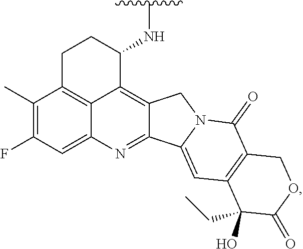 [SMILES: *N[C@H]1CCc2c(C)c(F)cc3nc4c(c1c23)Cn1c-4cc2c(c1=O)COC(=O)[C@]2(O)CC]

Dxd, Sn-38, monomethyl auristatin E (MMAE), and a pyrridinobenzodiazepine (PDD).

In one embodiment (CX), the disclosure provides a compound of formula (III), or salts, solvates, tautomers, isomers or mixtures thereof:

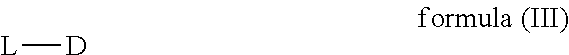

as defined in any one of embodiments (CI) to (CX), wherein index p is 8.

In one embodiment (CXI), the disclosure provides a compound of formula (III), or salts, solvates, tautomers, isomers or mixtures thereof:

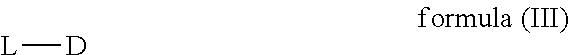

as defined in any of embodiments (CI) to (CX), wherein L₁ is —[CH₂]₂—C(O)NH—.

In one embodiment (CXII), the disclosure provides a compound of formula (III), or salts, solvates, tautomers, isomers or mixtures thereof:

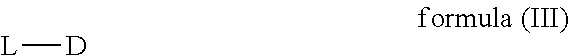

as defined in any one of embodiments (CI) to (CXI), wherein the drug moiety is selected from exatecan having the formula:

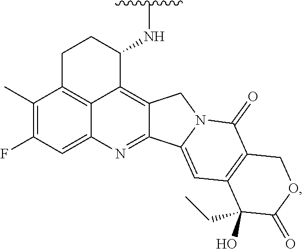 [SMILES: *N[C@H]1CCc2c(C)c(F)cc3nc4c(c1c23)Cn1c-4cc2c(c1=O)COC(=O)[C@]2(O)CC]

### Dxd, and SN-38.

In one embodiment (XIII), the disclosure provides a compound of formula (III), or salts, solvates, tautomers, isomers or mixtures thereof:

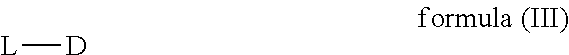

as defined in any one of embodiments (CI) to (CXII), wherein the drug moiety is selected from exatecan having the formula:

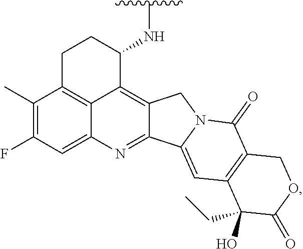 [SMILES: *N[C@H]1CCc2c(C)c(F)cc3nc4c(c1c23)Cn1c-4cc2c(c1=O)COC(=O)[C@]2(O)CC]

### Dxd, and SN-38.

In one embodiment (XIV), the disclosure provides a compound of formula (III), or salts, solvates, tautomers, isomers or mixtures thereof:

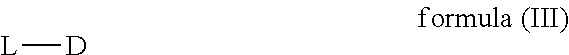

as defined in any one of embodiments (CI) to (CXIII), wherein the drug moiety is exatecan having the formula:

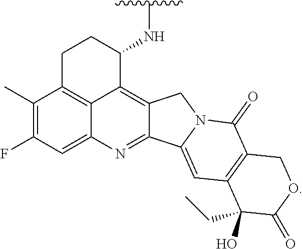 [SMILES: *N[C@H]1CCc2c(C)c(F)cc3nc4c(c1c23)Cn1c-4cc2c(c1=O)COC(=O)[C@]2(O)CC]

In one embodiment (CXV), the disclosure provides a compound of formula (III), or salts, solvates, tautomers, isomers or mixtures thereof:

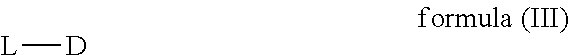

as defined in any one of embodiments (CI) to (CXIV), wherein X_{AA} is not Val-Cit or Phe-Lys.

In one embodiment (CXVI), the disclosure provides a compound of formula (III), or salts, solvates, tautomers, isomers or mixtures thereof:

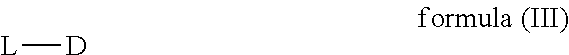

wherein the moiety L-D has the following structure

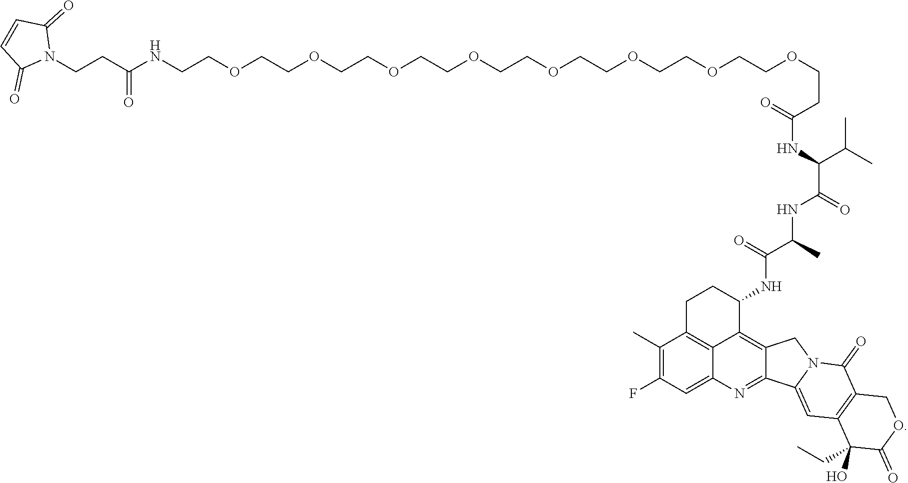 [SMILES: CC[C@@]1(O)C(=O)OCc2c1cc1n(c2=O)Cc2c-1nc1cc(F)c(C)c3c1c2[C@@H](NC(=O)[C@H](C)NC(=O)[C@@H](NC(=O)CCOCCOCCOCCOCCOCCOCCOCCOCCNC(=O)CCN1C(=O)C=CC1=O)C(C)C)CC3]

In one aspect, the disclosure provides a conjugate of formula (II):

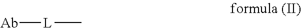

wherein Ab is an antibody or antibody fragment, L is a linker.

Any antibody, antibody fragment, and/or linker disclosed herein is contemplated within formula (II). In some embodiments, the linker comprises and/or consists of a partial structure, which is further conjugated to a drug moiety. In some embodiments, the linker comprises and/or consists of a complete structure, which can be further conjugated to a drug moiety.

In some embodiments, the linker comprises or consists of the formula:

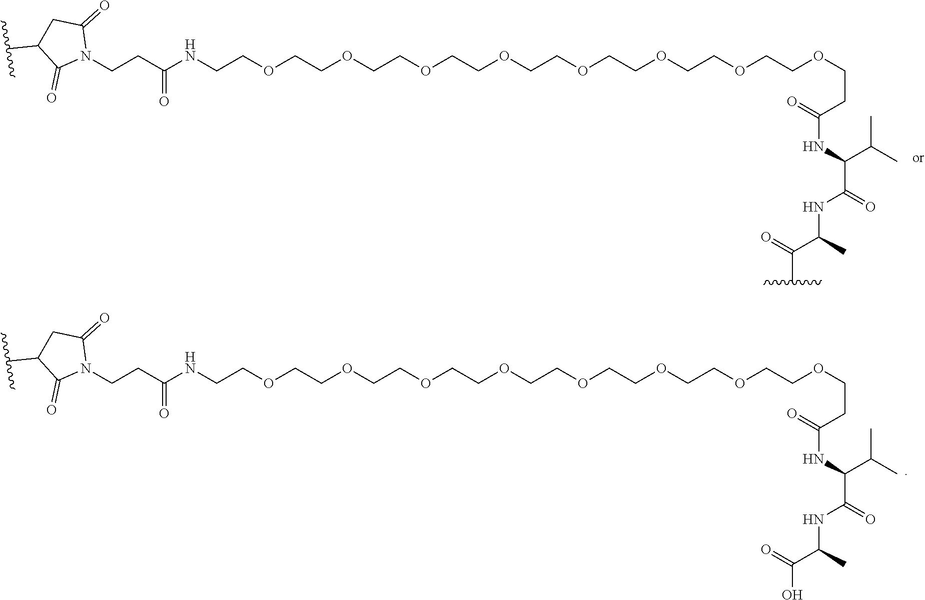 [SMILES: *C(=O)[C@H](C)NC(=O)[C@@H](NC(=O)CCOCCOCCOCCOCCOCCOCCOCCOCCNC(=O)CCN1C(=O)CC(*)C1=O)C(C)C ; *C1CC(=O)N(CCC(=O)NCCOCCOCCOCCOCCOCCOCCOCCOCCC(=O)N[C@H](C(=O)N[C@@H](C)C(=O)O)C(C)C)C1=O]

In some embodiments, the linker has the formula:

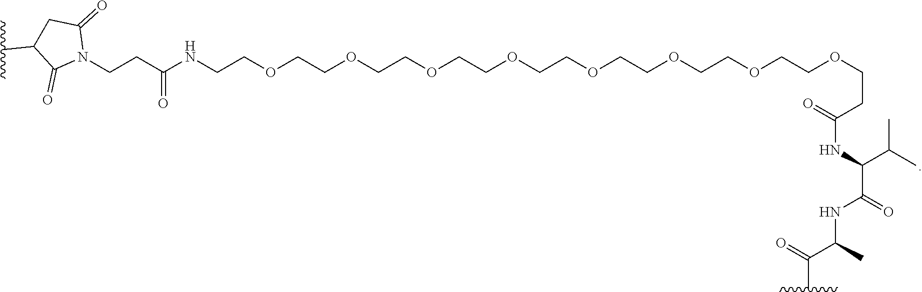 [SMILES: *C(=O)[C@H](C)NC(=O)[C@@H](NC(=O)CCOCCOCCOCCOCCOCCOCCOCCOCCNC(=O)CCN1C(=O)CC(*)C1=O)C(C)C]

In some embodiments, the linker has the formula:

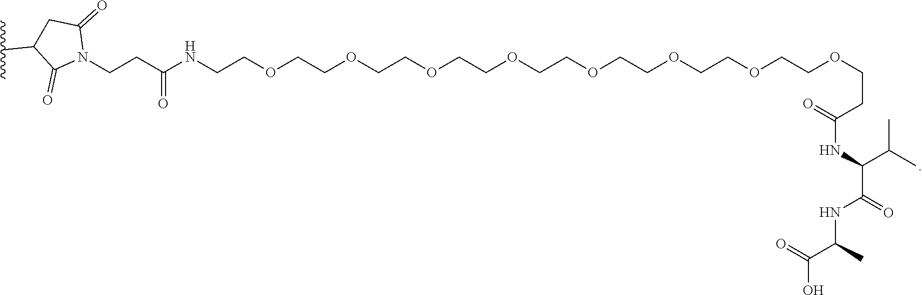 [SMILES: *C1CC(=O)N(CCC(=O)NCCOCCOCCOCCOCCOCCOCCOCCOCCC(=O)N[C@H](C(=O)N[C@@H](C)C(=O)O)C(C)C)C1=O]

In some embodiments, the linker comprises or consists of the formula:

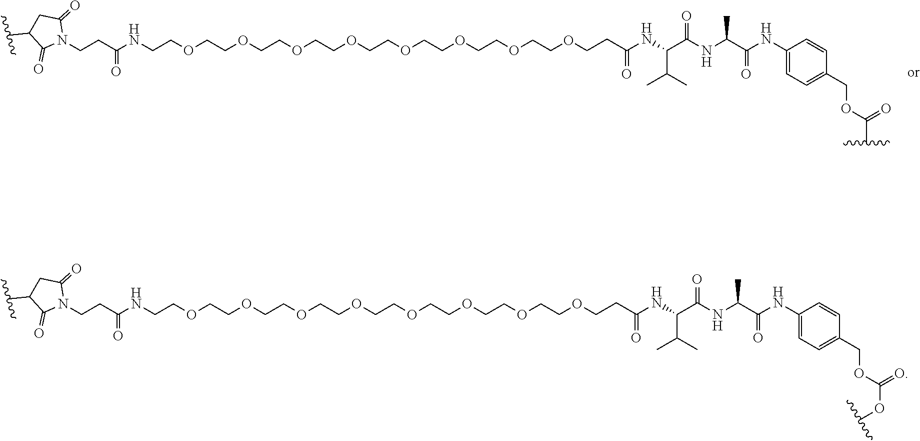 [SMILES: *C(=O)OCc1ccc(NC(=O)[C@H](C)NC(=O)[C@@H](NC(=O)CCOCCOCCOCCOCCOCCOCCOCCOCCNC(=O)CCN2C(=O)CC(*)C2=O)C(C)C)cc1 ; "or" ; *OC(=O)OCc1ccc(NC(=O)[C@H](C)NC(=O)[C@@H](NC(=O)CCOCCOCCOCCOCCOCCOCCOCCOCCNC(=O)CCN2C(=O)CC(*)C2=O)C(C)C)cc1]

In some embodiments, the linker comprises or consists of the formula:

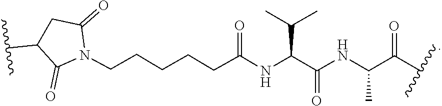 [SMILES: *C(=O)[C@H](C)NC(=O)[C@@H](NC(=O)CCCCCN1C(=O)CC(*)C1=O)C(C)C]

In some embodiments, the linker comprises or consists of the formula:

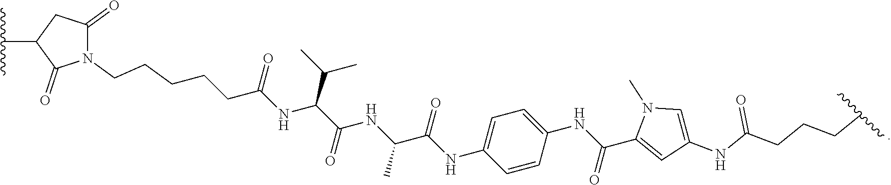 [SMILES: *CCCC(=O)Nc1cc(C(=O)Nc2ccc(NC(=O)[C@H](C)NC(=O)[C@@H](NC(=O)CCCCCN3C(=O)CC(*)C3=O)C(C)C)cc2)n(C)c1]

In some embodiments, the linker comprises or consists of the formula:

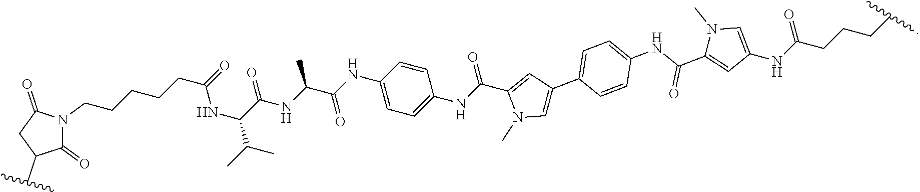 [SMILES: *CCCC(=O)Nc1cc(C(=O)Nc2ccc(-c3cc(C(=O)Nc4ccc(NC(=O)[C@H](C)NC(=O)[C@@H](NC(=O)CCCCCN5C(=O)CC(*)C5=O)C(C)C)cc4)n(C)c3)cc2)n(C)c1]

In some embodiments, the linker comprises or consists of the formula:

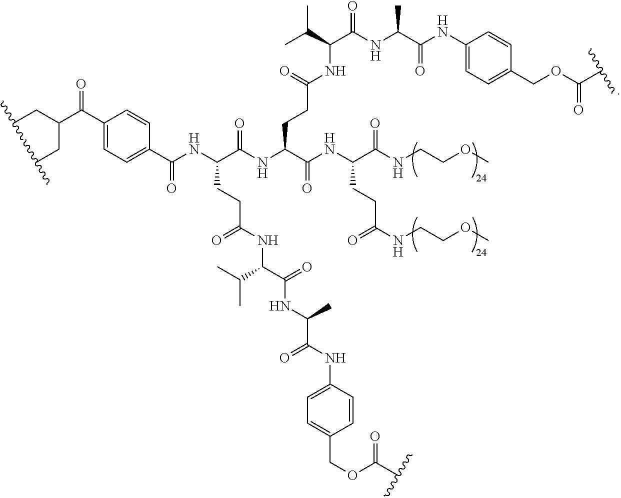

In some embodiments, the linker comprises or consists of the formula:

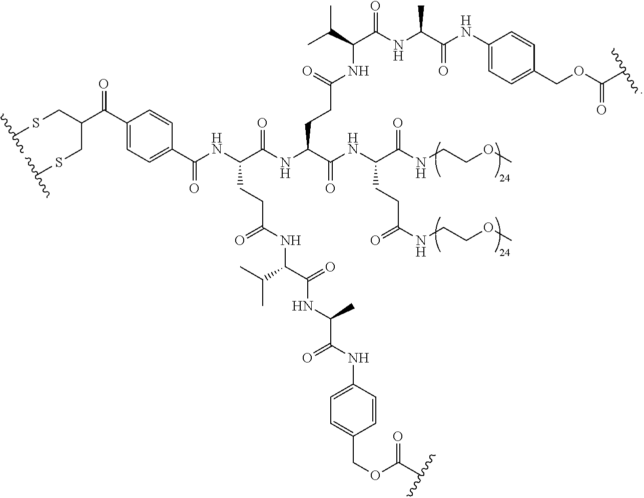

In some embodiments, the linker comprises or consists of the formula:

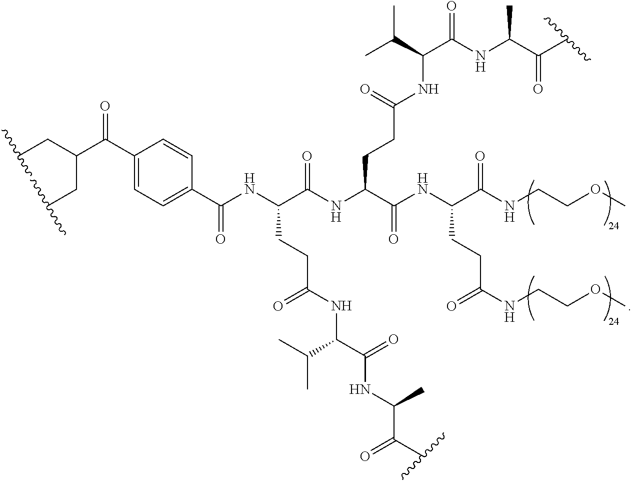

In some embodiments, the linker comprises or consists of the formula:

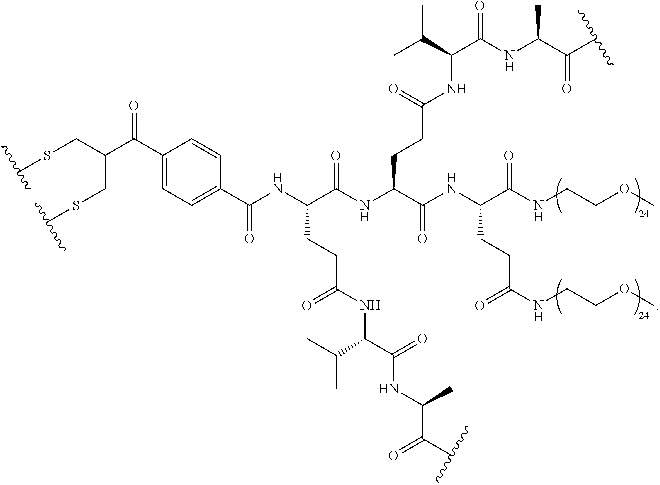

In some embodiments, the linker comprises or consists of the formula:

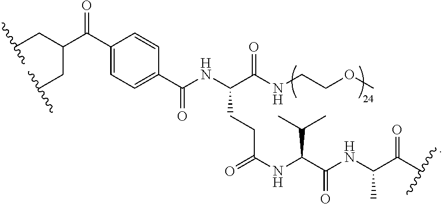

In some embodiments, the linker comprises or consists of the formula:

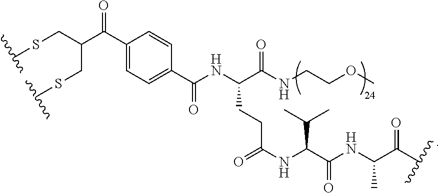

In some embodiments, the antibody or binding fragment thereof Ab specifically binds CUB Domain-Containing Protein-1 (CDCP1).

In some embodiments, the antibody, or binding fragment thereof Ab comprises:
(i) a heavy chain variable region (VH) that comprises one or more of:
   (a) a CDRH1 comprising and/or consisting of the amino acid sequence of SEQ ID NO: 2,
   (b) a CDRH2 comprising and/or consisting of the amino acid sequence of SEQ ID NO: 3, and
   (c) a CDRH3 comprising and/or consisting of the amino acid sequence of SEQ ID NO: 4,
and/or (ii) a light chain variable region (VL) that comprises one or more of:
   (a) a CDRL1 comprising and/or consisting of the amino acid sequence of SEQ ID NO: 6,
   (b) a CDRL2 comprising and/or consisting of the amino acid sequence of SEQ ID NO: 7, and
   (c) a CDRL3 comprising and/or consisting of the amino acid sequence of SEQ ID NO: 8.

In some embodiments, the antibody or binding fragment thereof Ab comprises a VH that comprises and/or consists of an amino acid sequence of SEQ ID NO: 1 and/or comprises a VL that comprises and/or consists of an amino acid sequence of SEQ ID NO: 5.

In some embodiments, the antibody or binding fragment thereof Ab comprises a heavy chain that comprises an amino acid sequence of SEQ ID NO: 1 and/or comprises a light chain that comprises an amino acid sequence of SEQ ID NO: 5.

### Antibody-Drug Conjugates

In one aspect, the disclosure provides an antibody-drug conjugate comprising one or more linkers and one or more drug moieties. In some embodiments, the antibody-drug conjugate comprises an antibody, antibody fragment, a linker and/or drug moiety described herein (e.g. formula (II) and/or formula (III)). In some embodiments, the antibody and/or antibody fragment is conjugated to a linker-drug moiety via a sulfur-containing moiety (e.g. thiol) on the antibody and/or antibody fragment. In some embodiments, the sulfur containing moiety comprises and/or consists of a sulfur moiety of one or more interchain disulfide bridges of the antibody and/or antibody fragment. In a non-limiting example, the interchain disulfide bridges holding the arms of the antibody (e.g. mAb) and/or antibody fragment together are broken using a reducing agent, and the linker and/or payload is conjugated to a sulfur moiety of the disulfide bridge. In a non-limiting example, all available thiols from interchain disulfides are occupied for a loading (DAR) of 8, due to the presence of 4 interchain disulfides in an antibody (e.g. mAb). In some embodiments, the antibody and/or antibody fragment is conjugated to a linker-drug moiety via one or more amino acid residues on the antibody and/or antibody fragment, including but not limited to amino acid residues comprising sulfur-containing side chains, (e.g. cysteine and/or methionine). In some embodiments, the antibody and/or antibody fragment is conjugated to a linker-drug moiety via one or more cysteine residues on the antibody and/or antibody fragment. In some embodiments, the antibody and/or antibody fragment is conjugated to a linker-drug moiety via one or more cysteine residues on the antibody. In some embodiments, the amino acid residue is non-engineered (e.g. a non-engineered cysteine and/or non-engineered methionine residue). In some embodiments, the amino acid residue is engineered (e.g. an engineered cysteine and/or engineered methionine residue). In some embodiments, the linker is selected from any of the linkers described herein. In some embodiments, the drug moiety is selected from any of the drug moieties described herein.

In one aspect, the disclosure provides an antibody-drug conjugate (ADC) having formula (I):

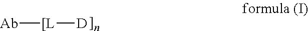

wherein in formula (I):
Ab comprises an antibody or antibody-binding fragment;
L is a linker;
D comprises a drug moiety; and
n is an integer from 1 to 20.

Any antibody, antibody fragment, linker, and/or drug moiety disclosed herein is contemplated within formula (III).

In some embodiments, the linker L of formula (I) is a linker of formula (III) that has reacted with antibody or antibody-binding fragment Ab (e.g. the linker comprises a reactive group R* which reacts with the antibody or antibody fragment Ab).

In some embodiments, the linker comprises or consists of the formula:

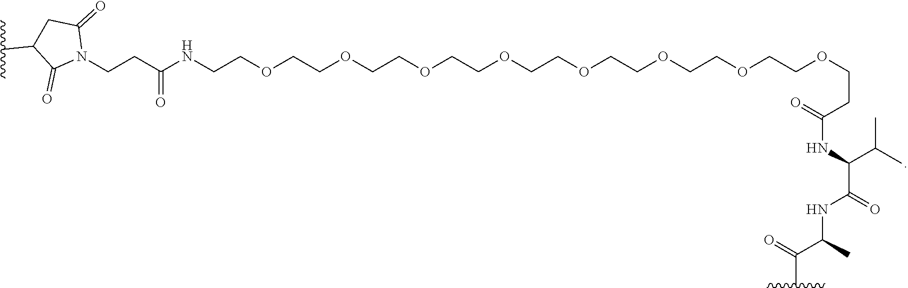 [SMILES: *C(=O)[C@H](C)NC(=O)[C@@H](NC(=O)CCOCCOCCOCCOCCOCCOCCOCCOCCNC(=O)CCN1C(=O)CC(*)C1=O)C(C)C]

In some embodiments, the linker comprises or consists of the formula:

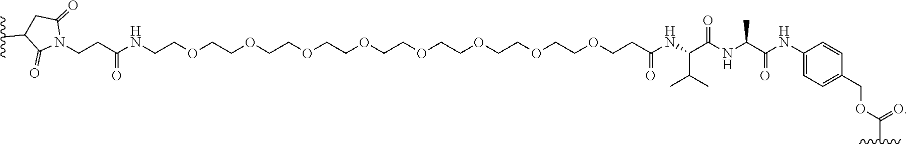 [SMILES: *C(=O)OCc1ccc(NC(=O)[C@H](C)NC(=O)[C@@H](NC(=O)CCOCCOCCOCCOCCOCCOCCOCCOCCNC(=O)CCN2C(=O)CC(*)C2=O)C(C)C)cc1]

In some embodiments, the linker comprises or consists of the formula:

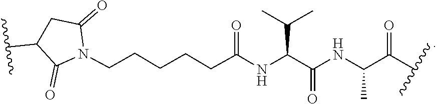 [SMILES: *C(=O)[C@H](C)NC(=O)[C@@H](NC(=O)CCCCCN1C(=O)CC(*)C1=O)C(C)C]

In some embodiments, the linker comprises or consists of the formula:

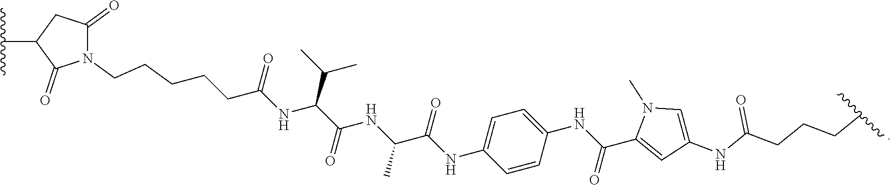 [SMILES: *CCCC(=O)Nc1cc(C(=O)Nc2ccc(NC(=O)[C@H](C)NC(=O)[C@@H](NC(=O)CCCCCN3C(=O)CC(*)C3=O)C(C)C)cc2)n(C)c1]

In some embodiments, the linker comprises or consists of the formula:

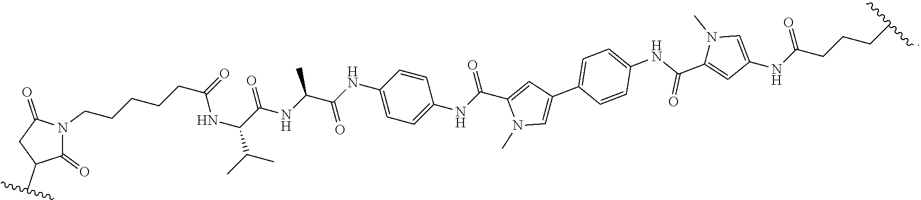 [SMILES: *CCCC(=O)Nc1cc(C(=O)Nc2ccc(-c3cc(C(=O)Nc4ccc(NC(=O)[C@H](C)NC(=O)[C@@H](NC(=O)CCCCCN5C(=O)CC(*)C5=O)C(C)C)cc4)n(C)c3)cc2)n(C)c1]

In some embodiments, the linker comprises or consists of the formula:

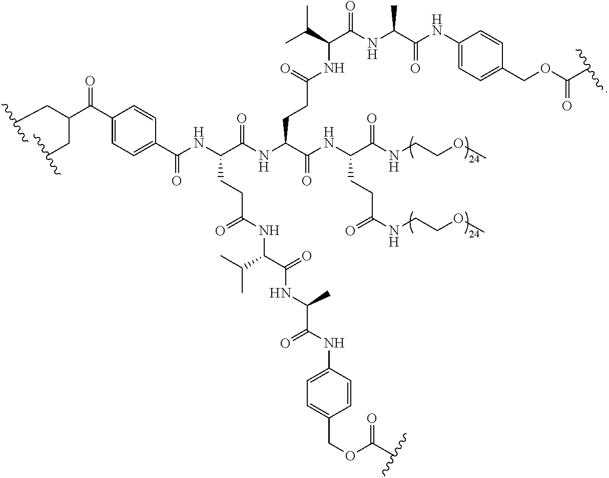

In some embodiments, the linker comprises or consists of the formula:

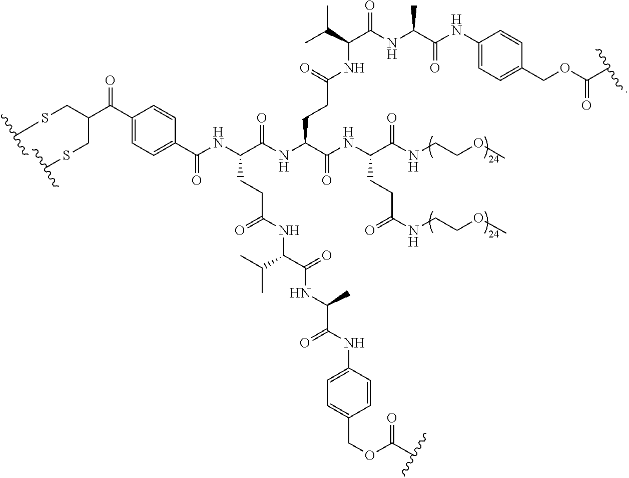

In some embodiments, the linker comprises or consists of the formula:

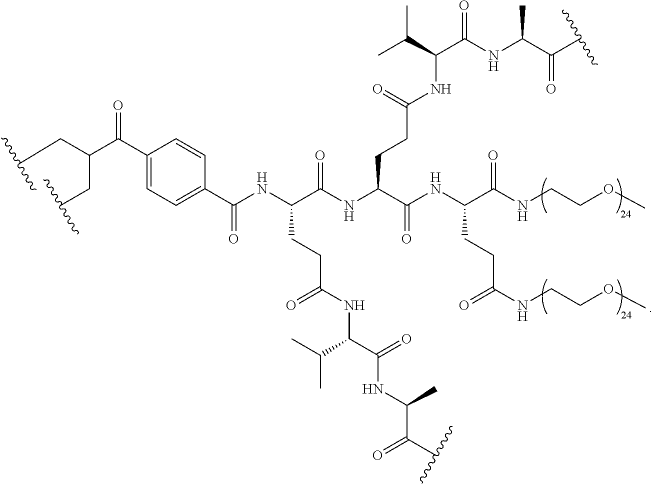

In some embodiments, the linker comprises or consists of the formula:

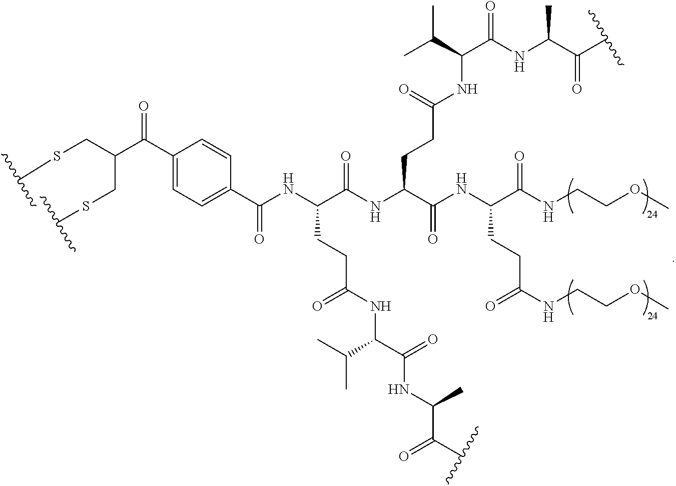

In some embodiments, the linker comprises or consists of the formula:

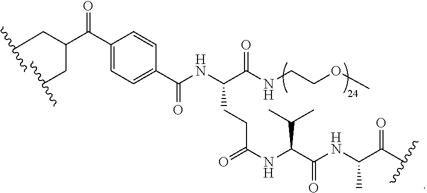

In some embodiments, the linker comprises or consists of the formula:

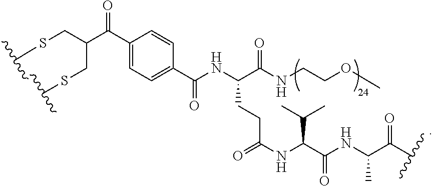

In some embodiments, the linker comprises or consists of valine-citrulline.

In some embodiments, the drug moiety D is selected from exatecan, Dxd, Sn-38, monomethyl auristatin E (MMAE), and pyrridinobenzodiazepines (PDDs). In some embodiments, the exatecan comprises or has the formula:

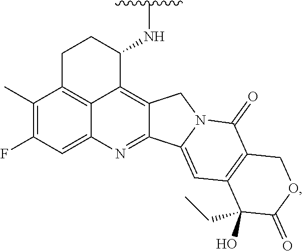 [SMILES: *N[C@H]1CCc2c(C)c(F)cc3nc4c(c1c23)Cn1c-4cc2c(c1=O)COC(=O)[C@]2(O)CC]

In some embodiments, the PDD comprises or has the formula:

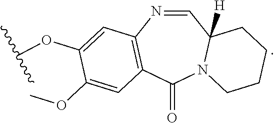 [SMILES: *Oc1cc2c(cc1OC)C(=O)N1CCCC[C@H]1C=N2]

In some embodiments, the PDD comprises or has the formula:

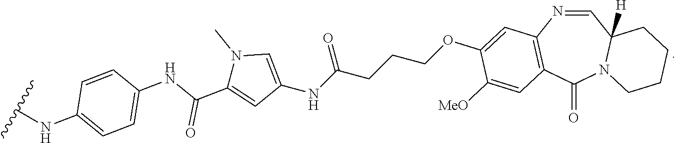 [SMILES: *Nc1ccc(NC(=O)c2cc(NC(=O)CCCOc3cc4c(cc3OC)C(=O)N3CCCC[C@H]3C=N4)cn2C)cc1]

In some embodiments, the PDD comprises or has the formula:

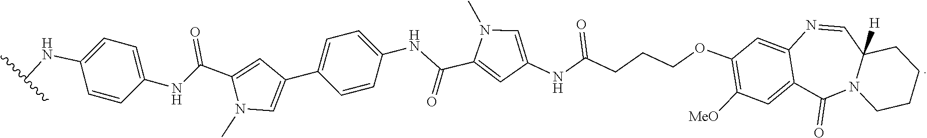 [SMILES: *Nc1ccc(NC(=O)c2cc(-c3ccc(NC(=O)c4cc(NC(=O)CCCOc5cc6c(cc5OC)C(=O)N5CCCC[C@H]5C=N6)cn4C)cc3)cn2C)cc1]

In some embodiments, the Sn-38 comprises or has the formula:

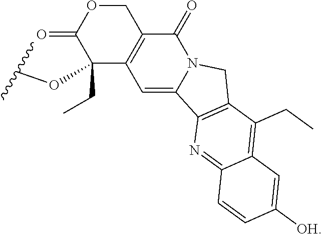 [SMILES: *O[C@]1(CC)C(=O)OCc2c1cc1n(c2=O)Cc2c-1nc1ccc(O)cc1c2CC]

In some embodiments, L-D has the formula:

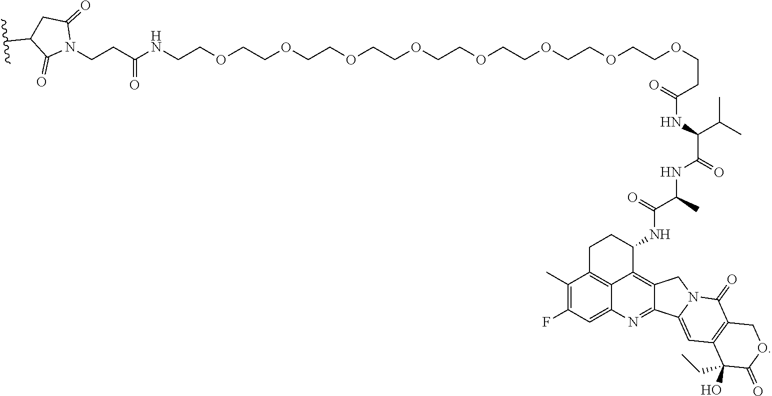 [SMILES: *C1CC(=O)N(CCC(=O)NCCOCCOCCOCCOCCOCCOCCOCCOCCC(=O)N[C@H](C(=O)N[C@@H](C)C(=O)N[C@H]2CCc3c(C)c(F)cc4nc5c(c2c34)Cn2c-5cc3c(c2=O)COC(=O)[C@]3(O)CC)C(C)C)C1=O]

In some embodiments, L-D has the formula:

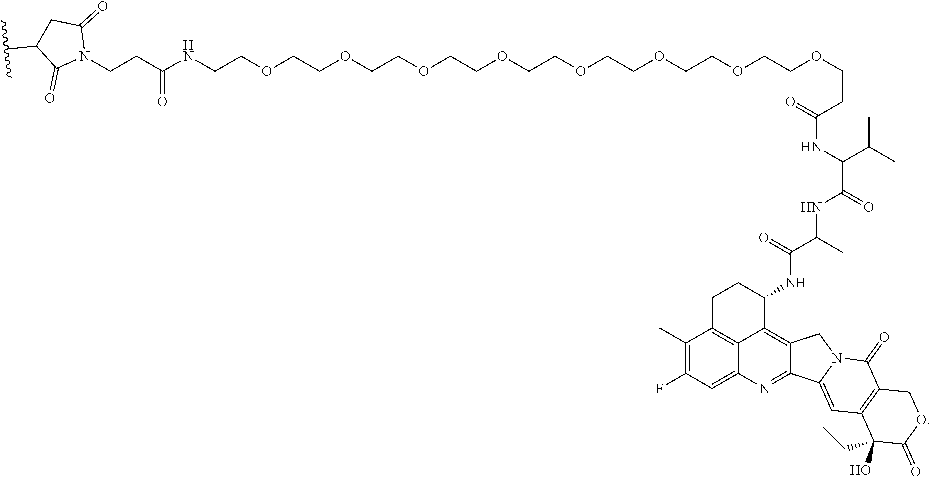 [SMILES: *C1CC(=O)N(CCC(=O)NCCOCCOCCOCCOCCOCCOCCOCCOCCC(=O)NC(C(=O)NC(C)C(=O)N[C@H]2CCc3c(C)c(F)cc4nc5c(c2c34)Cn2c-5cc3c(c2=O)COC(=O)[C@]3(O)CC)C(C)C)C1=O]

In some embodiments, L-D has the formula:

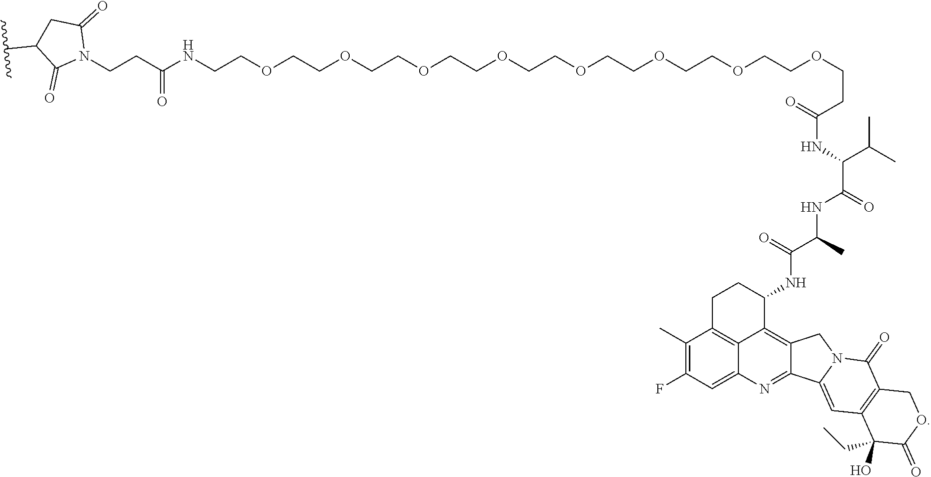 [SMILES: *C1CC(=O)N(CCC(=O)NCCOCCOCCOCCOCCOCCOCCOCCOCCC(=O)N[C@@H](C(=O)N[C@@H](C)C(=O)N[C@H]2CCc3c(C)c(F)cc4nc5c(c2c34)Cn2c-5cc3c(c2=O)COC(=O)[C@]3(O)CC)C(C)C)C1=O]

In some embodiments, L-D has the formula:

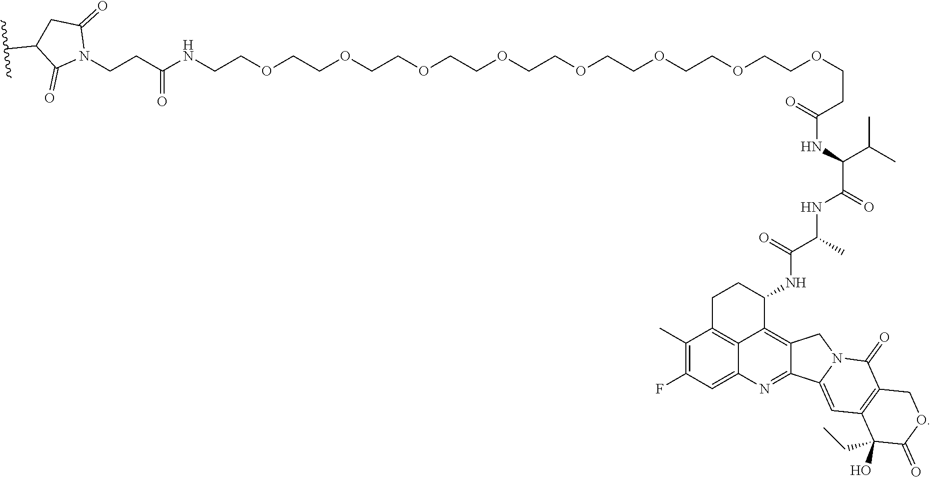 [SMILES: *C1CC(=O)N(CCC(=O)NCCOCCOCCOCCOCCOCCOCCOCCOCCC(=O)N[C@H](C(=O)N[C@H](C)C(=O)N[C@H]2CCc3c(C)c(F)cc4nc5c(c2c34)Cn2c-5cc3c(c2=O)COC(=O)[C@]3(O)CC)C(C)C)C1=O]

In some embodiments, L-D has the formula:

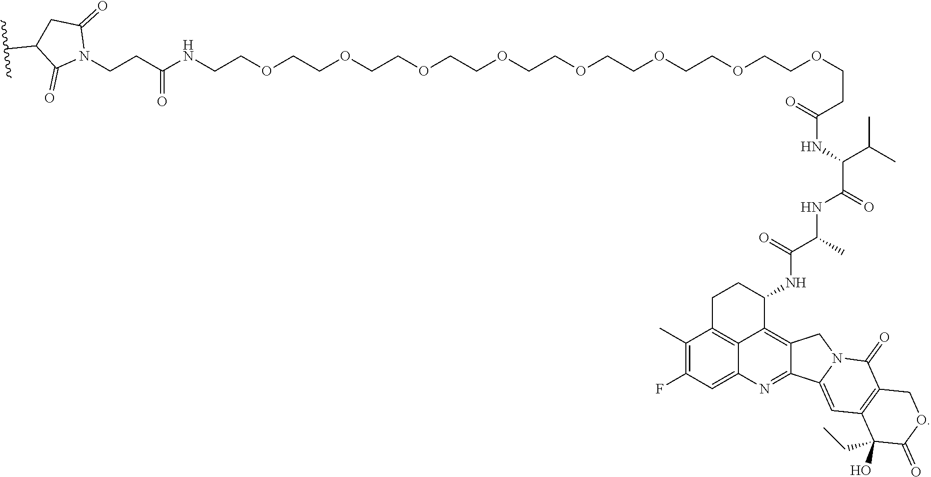 [SMILES: *C1CC(=O)N(CCC(=O)NCCOCCOCCOCCOCCOCCOCCOCCOCCC(=O)N[C@@H](C(=O)N[C@H](C)C(=O)N[C@H]2CCc3c(C)c(F)cc4nc5c(c2c34)Cn2c-5cc3c(c2=O)COC(=O)[C@]3(O)CC)C(C)C)C1=O]

In some embodiments, L-D has the formula:

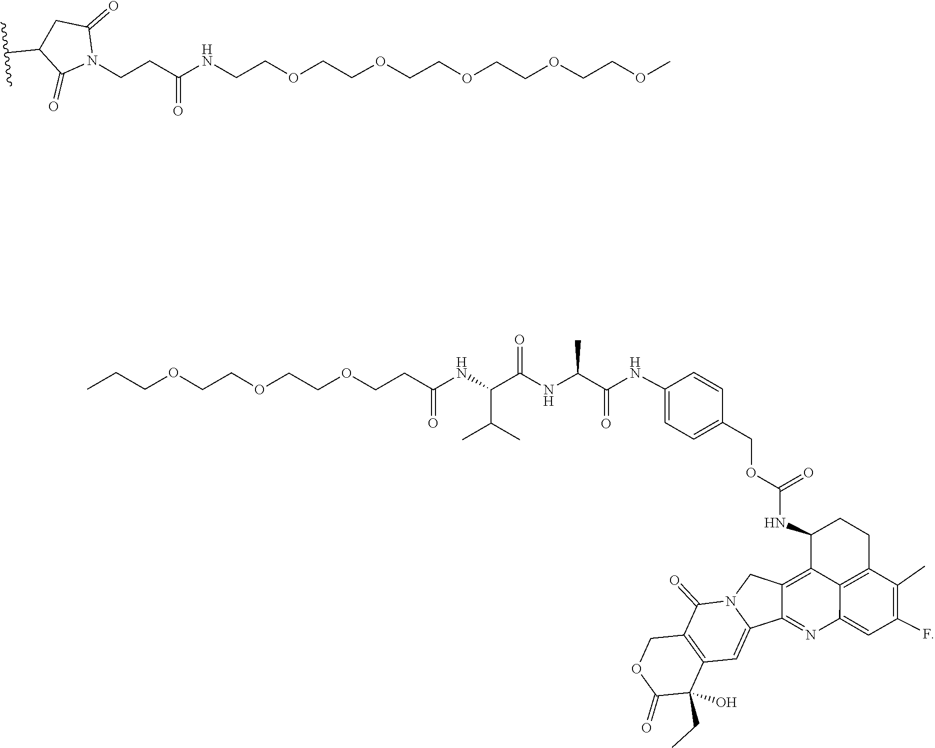

In some embodiments, L-D has the formula:

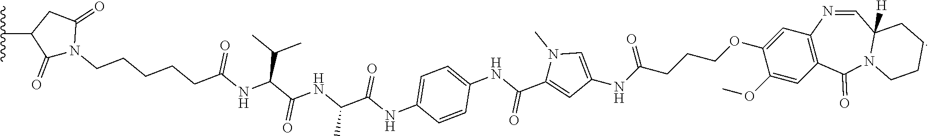 [SMILES: *C1CC(=O)N(CCCCCC(=O)N[C@H](C(=O)N[C@@H](C)C(=O)Nc2ccc(NC(=O)c3cc(NC(=O)CCCOc4cc5c(cc4OC)C(=O)N4CCCC[C@H]4C=N5)cn3C)cc2)C(C)C)C1=O]

In some embodiments, L-D has the formula:

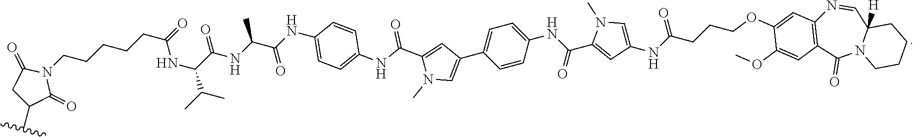 [SMILES: *C1CC(=O)N(CCCCCC(=O)N[C@H](C(=O)N[C@@H](C)C(=O)Nc2ccc(NC(=O)c3cc(-c4ccc(NC(=O)c5cc(NC(=O)CCCOc6cc7c(cc6OC)C(=O)N6CCCC[C@H]6C=N7)cn5C)cc4)cn3C)cc2)C(C)C)C1=O]

In some embodiments, L-D has the formula:

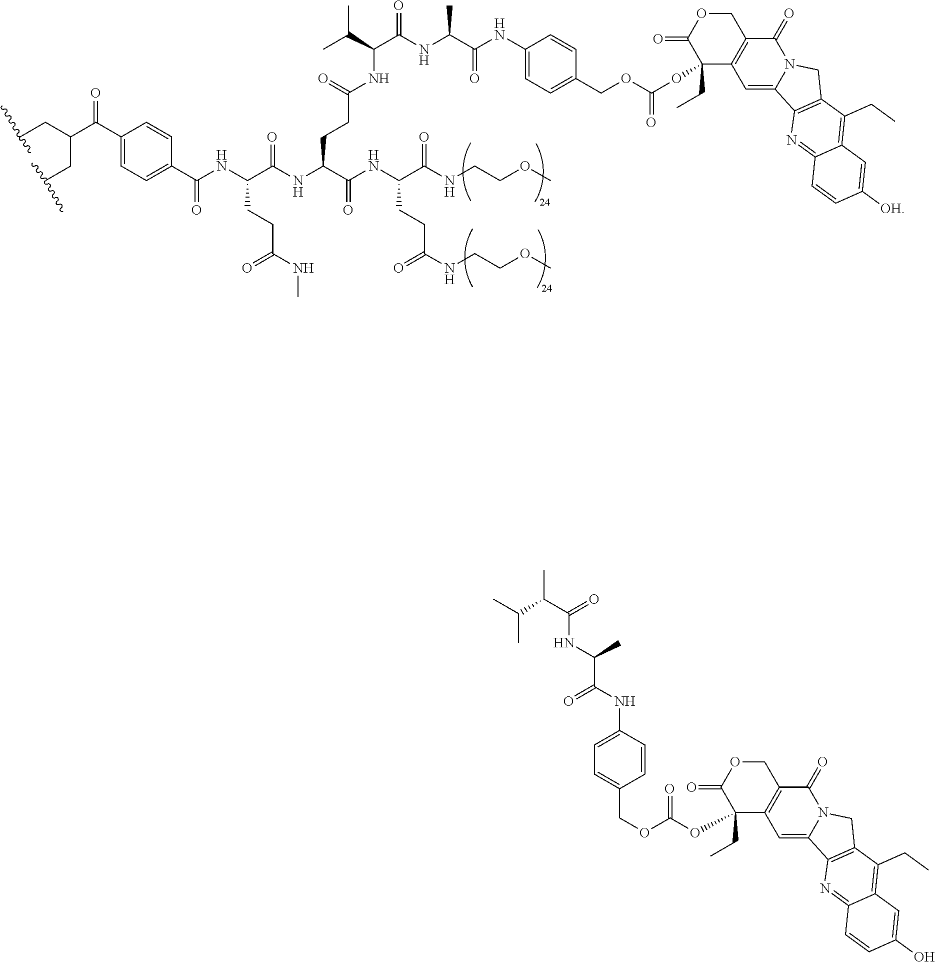

In some embodiments, L-D has the formula:

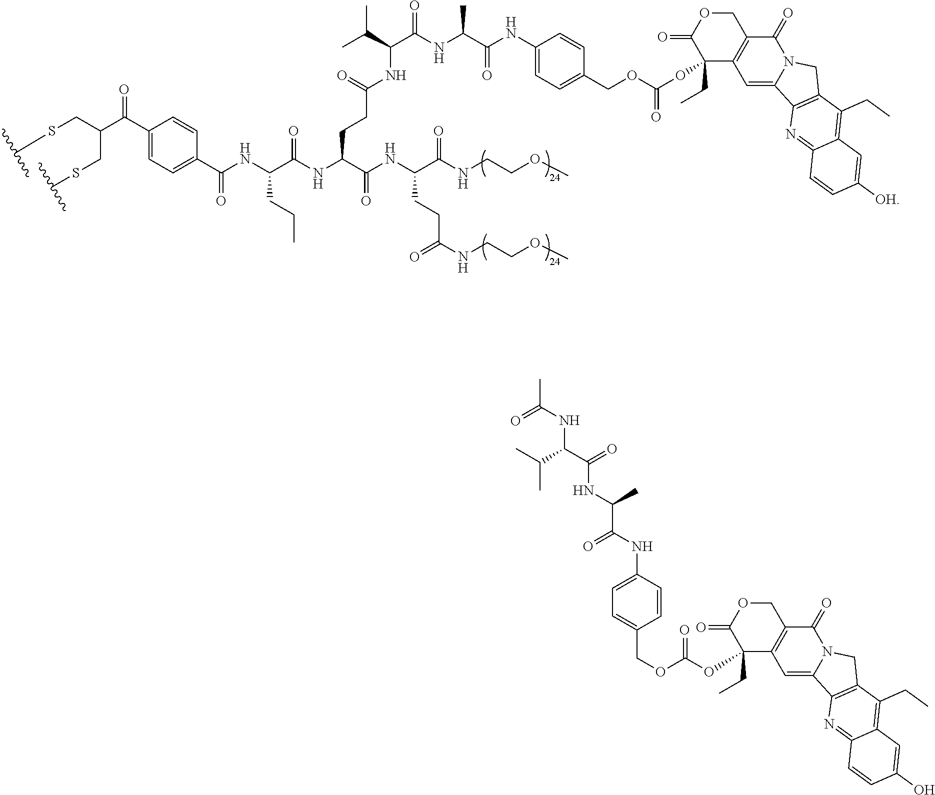

In some embodiments, L-D has the formula:

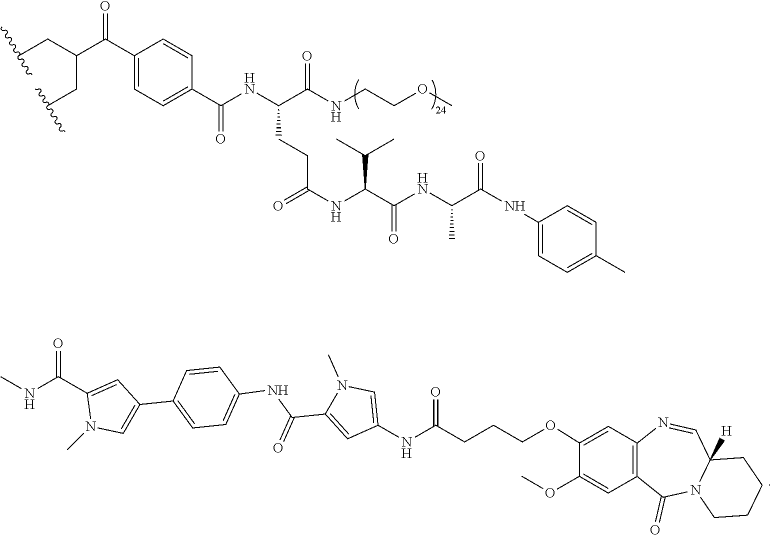

In some embodiments, L-D has the formula:

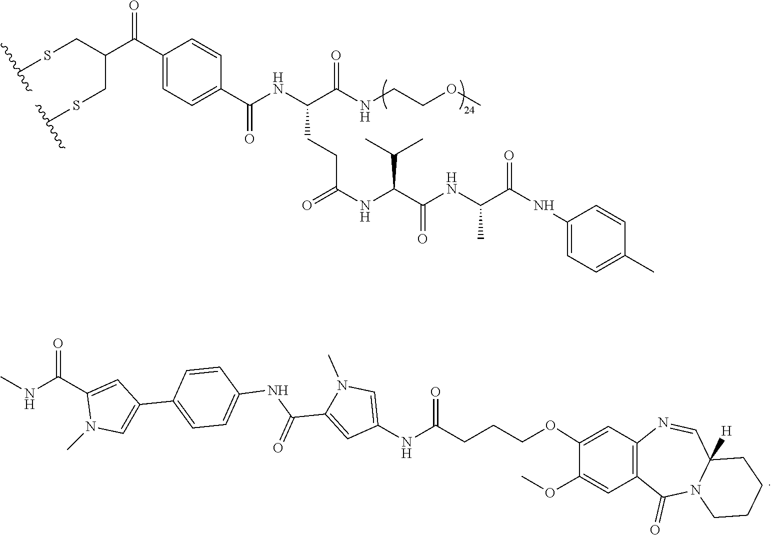

In some embodiments, L-D has the formula -val-cit-MMAE.

In some embodiments, L-D has the formula -val-cit.PAB-MMAE.

In some embodiments, the antibody-drug conjugate is of formula (I-A):

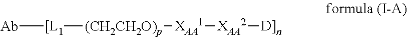

wherein in formula (I-A):
Ab comprises an antibody or antibody-binding fragment;
L₁ is

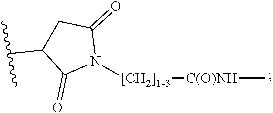

p is an integer from 6 to 20;
X_{AA}¹ and X_{AA}² are independently selected amino acid moieties;
D is a topoisomerase inhibitor; and
n is an integer from 1 to 20.

In some embodiments, the conjugate of formula (I) or the conjugate of formula (I-A) is selected from a conjugate of any one of formula 1030-1064:

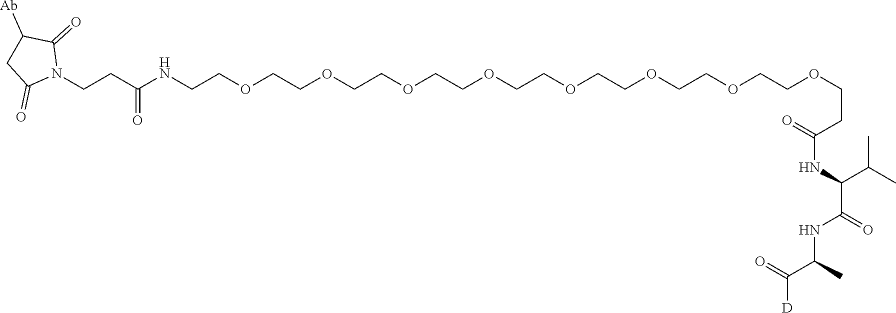

| Formula | D |
|---|---|
| 1030 | 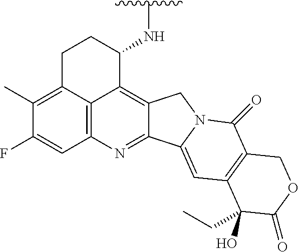 [SMILES: *N[C@H]1CCc2c(C)c(F)cc3nc4c(c1c23)Cn1c-4cc2c(c1=O)COC(=O)[C@]2(O)CC] |
| 1031 | 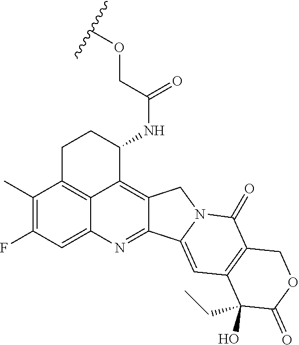 [SMILES: *OCC(=O)N[C@H]1CCc2c(C)c(F)cc3nc4c(c1c23)Cn1c-4cc2c(c1=O)COC(=O)[C@]2(O)CC] |
| 1032 | 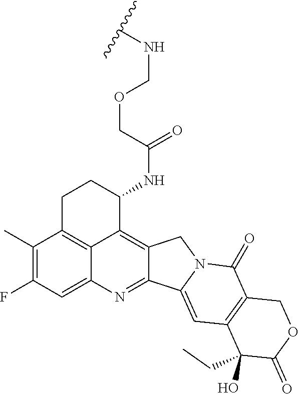 [SMILES: *NCOCC(=O)N[C@H]1CCc2c(C)c(F)cc3nc4c(c1c23)Cn1c-4cc2c(c1=O)COC(=O)[C@]2(O)CC] |
| 1033 | 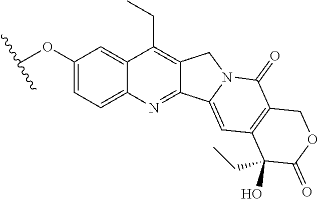 [SMILES: *Oc1ccc2nc3c(c(CC)c2c1)Cn1c-3cc2c(c1=O)COC(=O)[C@]2(O)CC] |
| 1034 | 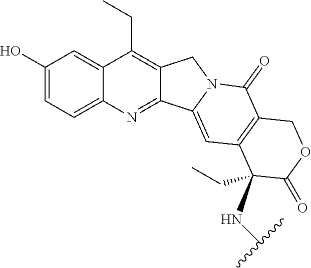 [SMILES: *N[C@]1(CC)C(=O)OCc2c1cc1n(c2=O)Cc2c-1nc1ccc(O)cc1c2CC] |
| 1035 | 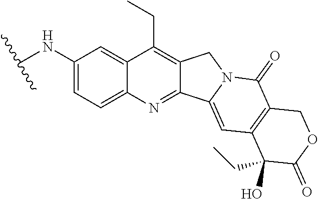 [SMILES: *Nc1ccc2nc3c(c(CC)c2c1)Cn1c-3cc2c(c1=O)COC(=O)[C@]2(O)CC] |
| 1036 | 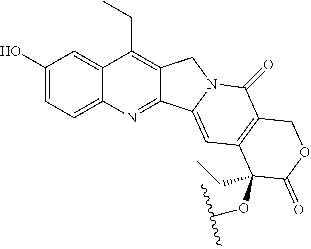 [SMILES: *O[C@]1(CC)C(=O)OCc2c1cc1n(c2=O)Cc2c-1nc1ccc(O)cc1c2CC] |
| 1037 | 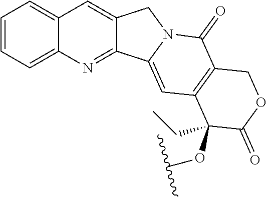 [SMILES: *O[C@]1(CC)C(=O)OCc2c1cc1n(c2=O)Cc2cc3ccccc3nc2-1] |
| 1038 | 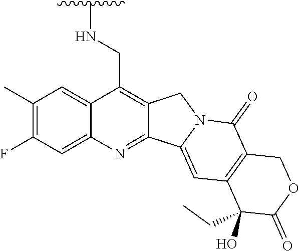 [SMILES: *NCc1c2c(nc3cc(F)c(C)cc13)-c1cc3c(c(=O)n1C2)COC(=O)[C@]3(O)CC] |
| 1039 | 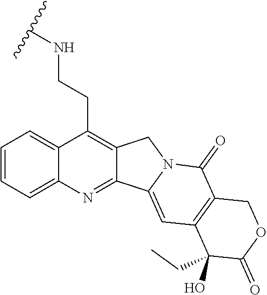 [SMILES: *NCCc1c2c(nc3ccccc13)-c1cc3c(c(=O)n1C2)COC(=O)[C@]3(O)CC] |
| 1040 | 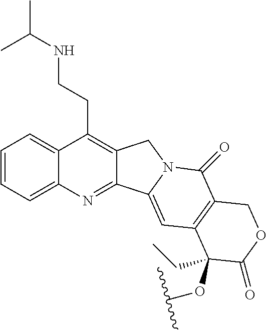 [SMILES: *O[C@]1(CC)C(=O)OCc2c1cc1n(c2=O)Cc2c-1nc1ccccc1c2CCNC(C)C] |
| 1041 | 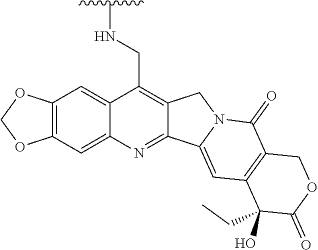 [SMILES: *NCc1c2c(nc3cc4c(cc13)OCO4)-c1cc3c(c(=O)n1C2)COC(=O)[C@]3(O)CC] |
| 1042 | 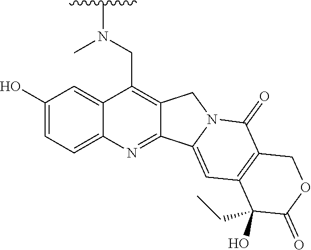 [SMILES: *N(C)Cc1c2c(nc3ccc(O)cc13)-c1cc3c(c(=O)n1C2)COC(=O)[C@]3(O)CC] |
| 1043 | 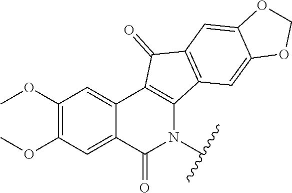 [SMILES: *n1c2c(c3cc(OC)c(OC)cc3c1=O)C(=O)c1cc3c(cc1-2)OCO3] |
| 1044 | 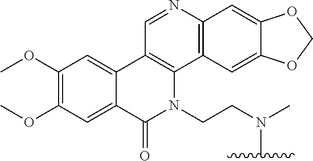 [SMILES: *N(C)CCn1c(=O)c2cc(OC)c(OC)cc2c2cnc3cc4c(cc3c21)OCO4] |
| 1045 | 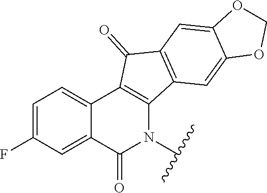 [SMILES: *n1c2c(c3ccc(F)cc3c1=O)C(=O)c1cc3c(cc1-2)OCO3] |
| 1046 | 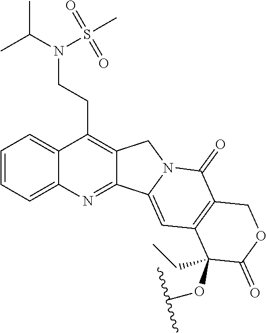 [SMILES: *O[C@]1(CC)C(=O)OCc2c1cc1n(c2=O)Cc2c-1nc1ccccc1c2CCN(C(C)C)S(C)(=O)=O] |
| 1047 | 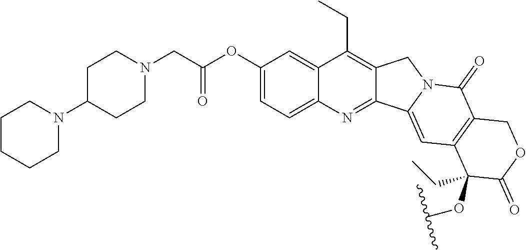 [SMILES: *O[C@]1(CC)C(=O)OCc2c1cc1n(c2=O)Cc2c-1nc1ccc(OC(=O)CN3CCC(N4CCCCC4)CC3)cc1c2CC] |
| 1048 | 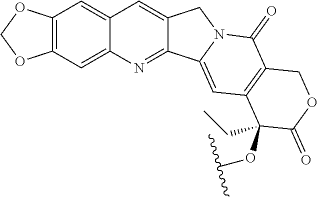 [SMILES: *O[C@]1(CC)C(=O)OCc2c1cc1n(c2=O)Cc2cc3cc4c(cc3nc2-1)OCO4] |
| 1049 | 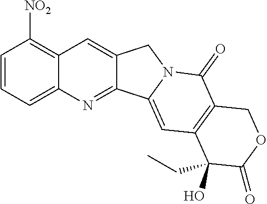 |
| 1050 | 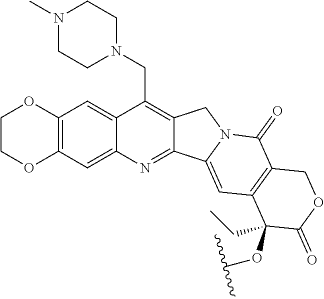 [SMILES: *O[C@]1(CC)C(=O)OCc2c1cc1n(c2=O)Cc2c-1nc1cc3c(cc1c2CN1CCN(C)CC1)OCCO3] |
| 1051 | 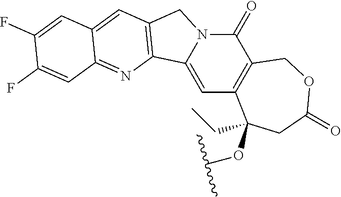 [SMILES: *O[C@]1(CC)CC(=O)OCc2c1cc1n(c2=O)Cc2cc3cc(F)c(F)cc3nc2-1] |
| 1052 | 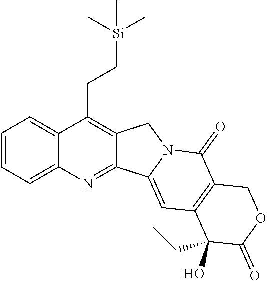 |
| 1053 | 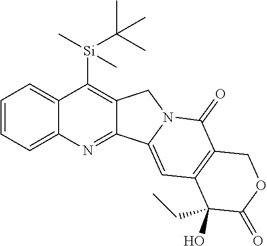 |
| 1054 | 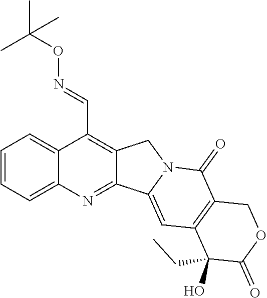 |
| 1055 | 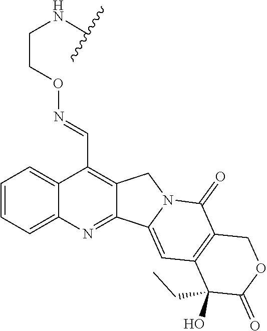 [SMILES: *NCCO/N=C/c1c2c(nc3ccccc13)-c1cc3c(c(=O)n1C2)COC(=O)[C@]3(O)CC] |
| 1056 | 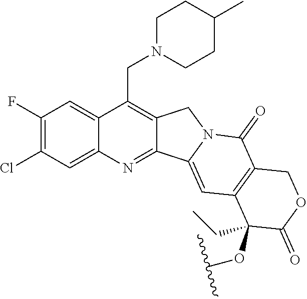 [SMILES: *O[C@]1(CC)C(=O)OCc2c1cc1n(c2=O)Cc2c-1nc1cc(Cl)c(F)cc1c2CN1CCC(C)CC1] |
| 1057 | 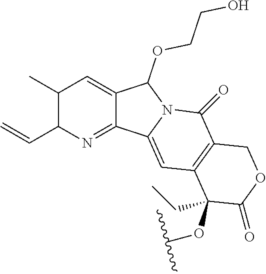 [SMILES: *O[C@]1(CC)C(=O)OCc2c1cc1n(c2=O)C(OCCO)C2=CC(C)C(C=C)N=C21] |
| 1058 | 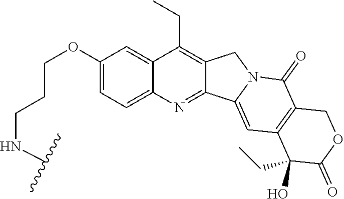 [SMILES: *NCCCOc1ccc2nc3c(c(CC)c2c1)Cn1c-3cc2c(c1=O)COC(=O)[C@]2(O)CC] |
| 1059 | 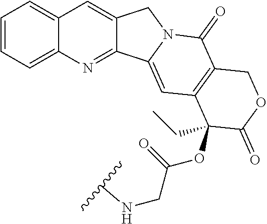 [SMILES: *NCC(=O)O[C@]1(CC)C(=O)OCc2c1cc1n(c2=O)Cc2cc3ccccc3nc2-1] |
| 1060 | 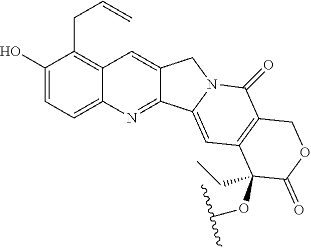 [SMILES: *O[C@]1(CC)C(=O)OCc2c1cc1n(c2=O)Cc2cc3c(CC=C)c(O)ccc3nc2-1] |
| 1061 | 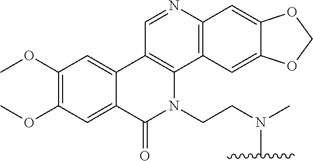 [SMILES: *N(C)CCn1c(=O)c2cc(OC)c(OC)cc2c2cnc3cc4c(cc3c21)OCO4] |
| 1062 | 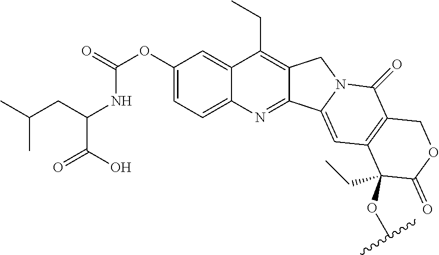 [SMILES: *O[C@]1(CC)C(=O)OCc2c1cc1n(c2=O)Cc2c-1nc1ccc(OC(=O)NC(CC(C)C)C(=O)O)cc1c2CC] |
| 1063 | 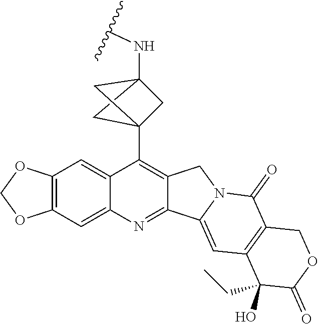 [SMILES: *NC12CC(c3c4c(nc5cc6c(cc35)OCO6)-c3cc5c(c(=O)n3C4)COC(=O)[C@]5(O)CC)(C1)C2] |
| 1064 | 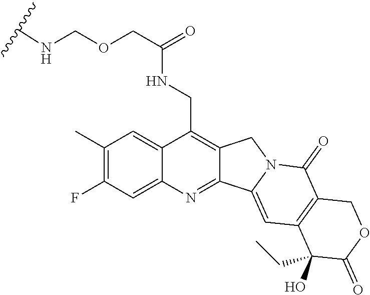 [SMILES: *NCOCC(=O)NCc1c2c(nc3cc(F)c(C)cc13)-c1cc3c(c(=O)n1C2)COC(=O)[C@]3(O)CC] |

In some embodiments, the conjugate of formula (I) or the conjugate of formula (I-A) is selected from a conjugate of any one of formula 1030 or 1100-1118:

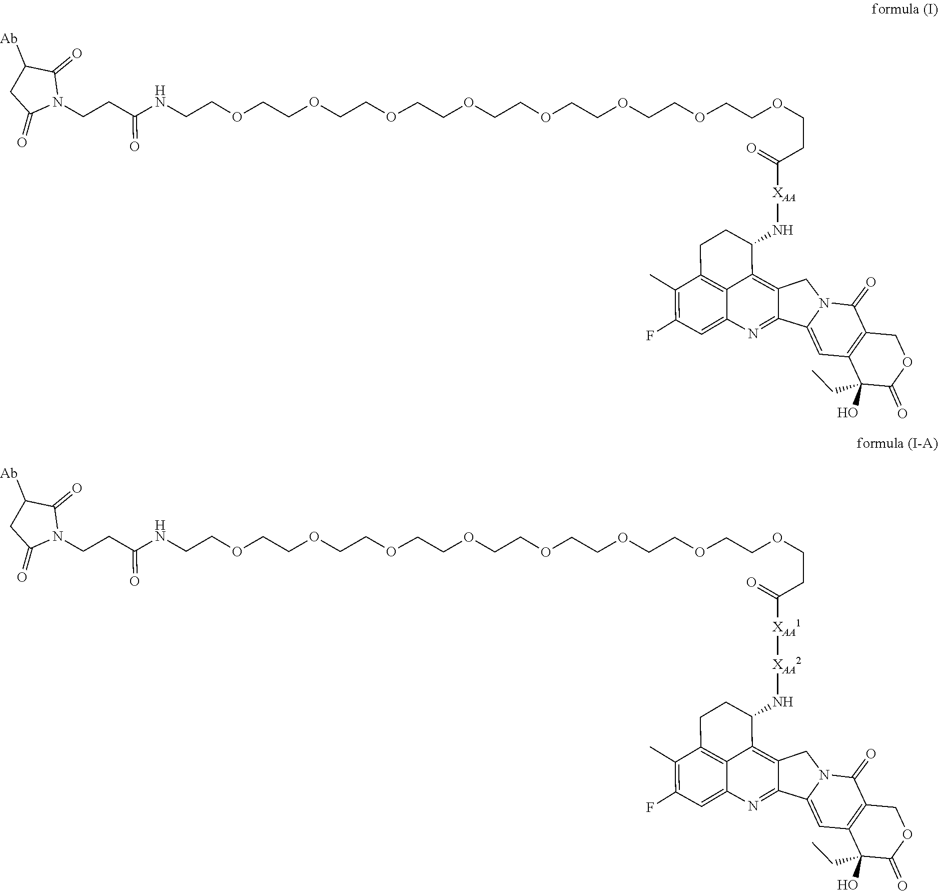

| Formula | -X_{AA}- or -X_{AA}¹-X_{AA}²- |
|---|---|
| 1030 | Val-Ala |
| 1101 | Tyr-Arg |
| 1102 | Phe-Arg |
| 1103 | Val-Gln |
| 1104 | Val-Cit |
| 1105 | Tyr-Met |
| 1106 | Leu-Gln |
| 1107 | Val-Arg |
| 1108 | Met-Thr |
| 1109 | Phe-Gln |
| 1110 | Thr-Thr |
| 1111 | Val-Thr |
| 1112 | Ala-Ala |
| 1113 | Val-Met |
| 1114 | Leu-Met |
| 1115 | Ala-Asn |
| 1116 | D-Val-D-Gln |
| 1117 | D-Ala-D-Ala |
| 1118 | Phe-Met |

In some embodiments, n is an integer from 1 to 10. In some embodiments, n is an integer from 4 to 8. In some embodiments, n is an integer from 2 to 8. In some embodiments, n is 1. In some embodiments, n is 2. In some embodiments, n is 3. In some embodiments, n is 4. In some embodiments, n is 5. In some embodiments, n is 6. In some embodiments, n is 7. In some embodiments, n is 8. In some embodiments, n is 9. In some embodiments, n is 10.

In one embodiment (I), the disclosure provides an antibody-drug conjugate having formula (I):

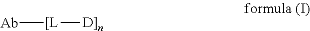

wherein in formula (I):
Ab is an antibody or antibody-binding fragment;
L is a linker;
D is a drug moiety; and
n is an integer from 1 to 20.
   The linker has the following formula:

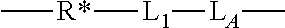

wherein L_{A} is a linker, L₁ is a linking moiety, and R* is a reactive moiety or a targeting agent. The linker L_{A} has the formula:

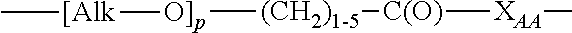

wherein
Alk designates C₂-C₄-alkylene,
X_{AA} is a dipeptide,
p is an integer from 0 to 50.
   R* is a reactive moiety selected from a succinimide, triazole, an amide, and a thioether.
   L₁ is —[CH₂]₀₋₁₂—C(O)NH—.
   The drug moiety is selected from exatecan having the formula:

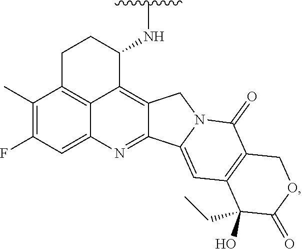 [SMILES: *N[C@H]1CCc2c(C)c(F)cc3nc4c(c1c23)Cn1c-4cc2c(c1=O)COC(=O)[C@]2(O)CC]

Dxd, Sn-38, monomethyl auristatin E (MMAE), and a pyrridinobenzodiazepine (PDD).

In one embodiment (I), the disclosure provides an antibody-drug conjugate having formula (I):

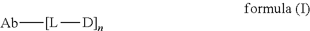

wherein in formula (I):
Ab is an antibody or antibody-binding fragment;
L is a linker;
D is a drug moiety; and
n is an integer from 1 to 20.
   The linker has the following formula:

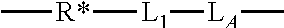

wherein L_{A} is a linker, L₁ is a linking moiety, and R* is a reactive moiety or a targeting agent. The linker L_{A} has the formula:

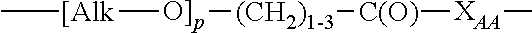

wherein
Alk designates C₂-C₄-alkylene,
X_{AA} is a dipeptide,
p is an integer from 0 to 50.
   R* is a reactive moiety selected from a succinimide, triazole, an amide, and a thioether.
   L₁ is —[CH₂]₀₋₁₂—C(O)NH—.
   The drug moiety is selected from exatecan having the formula:

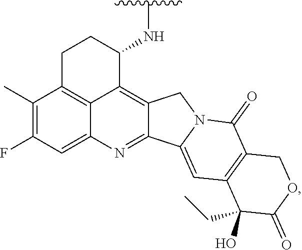 [SMILES: *N[C@H]1CCc2c(C)c(F)cc3nc4c(c1c23)Cn1c-4cc2c(c1=O)COC(=O)[C@]2(O)CC]

Dxd, Sn-38, monomethyl auristatin E (MMAE), and a pyrridinobenzodiazepine (PDD).

In one embodiment (Ib), the disclosure provides an antibody-drug conjugate having formula (I):

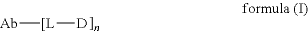

wherein in formula (I):
Ab is an antibody or antibody-binding fragment;
L is a linker;
D is a drug moiety; and
n is an integer from 1 to 20.
   The linker has the following formula:

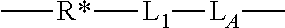

wherein L_{A} is a linker, L₁ is a linking moiety, and R* is a reactive moiety or a targeting agent.
The linker L_{A} has the formula:

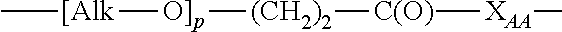

wherein
Alk designates C₂-C₄-alkylene,
X_{AA} is a dipeptide,
p is an integer from 0 to 50.
   R* is a reactive moiety selected from a succinimide, triazole, an amide, and a thioether.
   L₁ is —[CH₂]₀₋₁₂—C(O)NH—.
   The drug moiety is selected from exatecan having the formula:

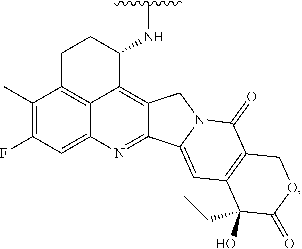 [SMILES: *N[C@H]1CCc2c(C)c(F)cc3nc4c(c1c23)Cn1c-4cc2c(c1=O)COC(=O)[C@]2(O)CC]

Dxd, Sn-38, monomethyl auristatin E (MMAE), and a pyrridinobenzodiazepine (PDD).

In one embodiment (Ic), the disclosure provides an antibody-drug conjugate having formula (I):

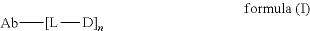

wherein in formula (I):
Ab is an antibody or antibody-binding fragment;
L is a linker;
D is a drug moiety; and
n is an integer from 1 to 20.
   The linker has the following formula:

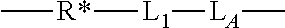

wherein L_{A} is a linker, L₁ is a linking moiety, and R* is a reactive moiety or a targeting agent.
The linker L_{A} has the formula:

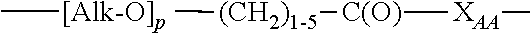

wherein
Alk designates C₂-C₄-alkylene,
X_{AA} is a dipeptide,
p is an integer from 0 to 50.
   R* is a reactive moiety selected from a succinimide, triazole, an amide, and a thioether.
   L₁ is —[CH₂]₀₋₁₂—C(O)NH—.
   The drug moiety is selected from:

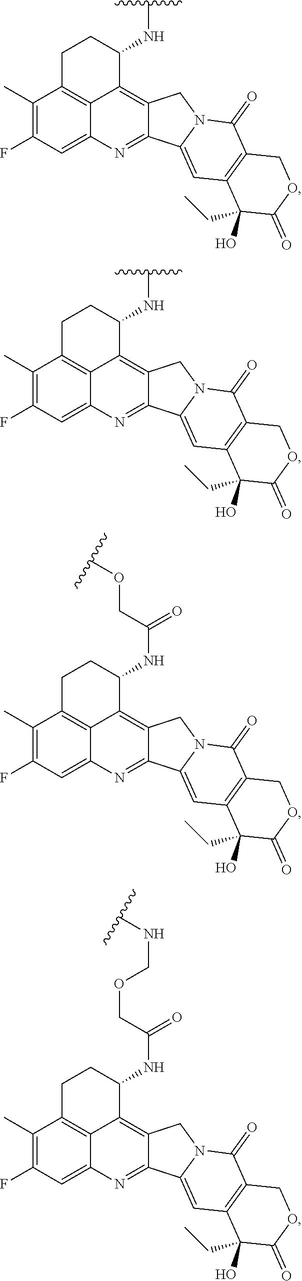 [SMILES: *N[C@H]1CCc2c(C)c(F)cc3nc4c(c1c23)Cn1c-4cc2c(c1=O)COC(=O)[C@]2(O)CC ; *N[C@H]1CCc2c(C)c(F)cc3nc4c(c1c23)Cn1c-4cc2c(c1=O)COC(=O)[C@]2(O)CC ; *OCC(=O)N[C@H]1CCc2c(C)c(F)cc3nc4c(c1c23)Cn1c-4cc2c(c1=O)COC(=O)[C@]2(O)CC ; *NCOCC(=O)N[C@H]1CCc2c(C)c(F)cc3nc4c(c1c23)Cn1c-4cc2c(c1=O)COC(=O)[C@]2(O)CC]

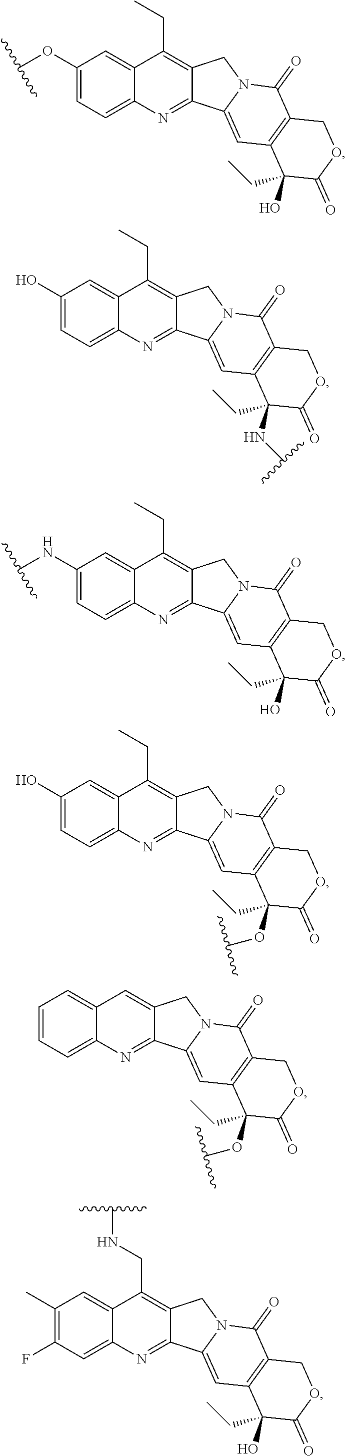 [SMILES: *Oc1ccc2nc3c(c(CC)c2c1)Cn1c-3cc2c(c1=O)COC(=O)[C@]2(O)CC ; *N[C@]1(CC)C(=O)OCc2c1cc1n(c2=O)Cc2c-1nc1ccc(O)cc1c2CC ; *Nc1ccc2nc3c(c(CC)c2c1)Cn1c-3cc2c(c1=O)COC(=O)[C@]2(O)CC ; *O[C@]1(CC)C(=O)OCc2c1cc1n(c2=O)Cc2c-1nc1ccc(O)cc1c2CC ; *O[C@]1(CC)C(=O)OCc2c1cc1n(c2=O)Cc2cc3ccccc3nc2-1 ; *NCc1c2c(nc3cc(F)c(C)cc13)-c1cc3c(c(=O)n1C2)COC(=O)[C@]3(O)CC]

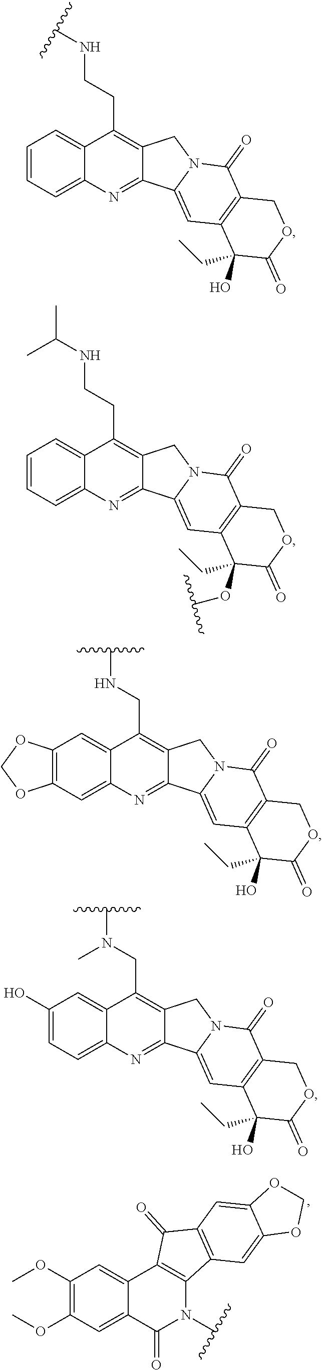

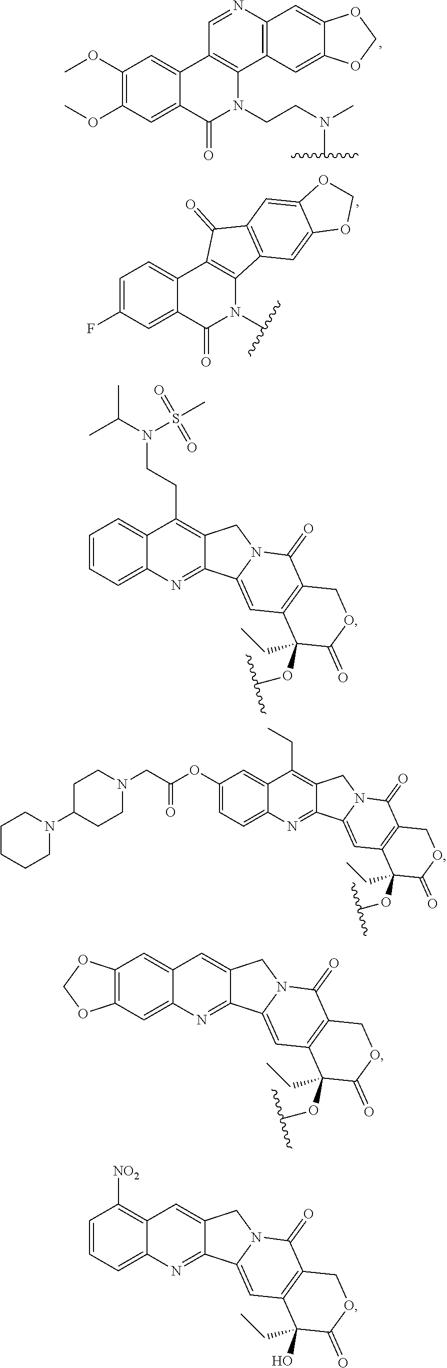

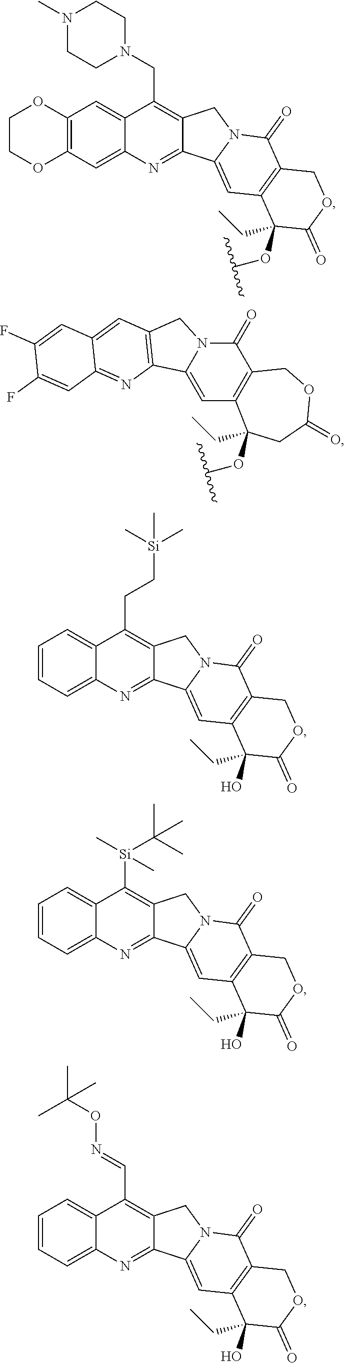 [SMILES: *O[C@]1(CC)C(=O)OCc2c1cc1n(c2=O)Cc2c-1nc1cc3c(cc1c2CN1CCN(C)CC1)OCCO3 ; *O[C@]1(CC)CC(=O)OCc2c1cc1n(c2=O)Cc2cc3cc(F)c(F)cc3nc2-1 ; CC[C@@]1(O)C(=O)OCc2c1cc1n(c2=O)Cc2c-1nc1ccccc1c2CC[Si](C)(C)C ; CC[C@@]1(O)C(=O)OCc2c1cc1n(c2=O)Cc2c-1nc1ccccc1c2[Si](C)(C)C(C)(C)C ; CC[C@@]1(O)C(=O)OCc2c1cc1n(c2=O)Cc2c-1nc1ccccc1c2/C=N/OC(C)(C)C]

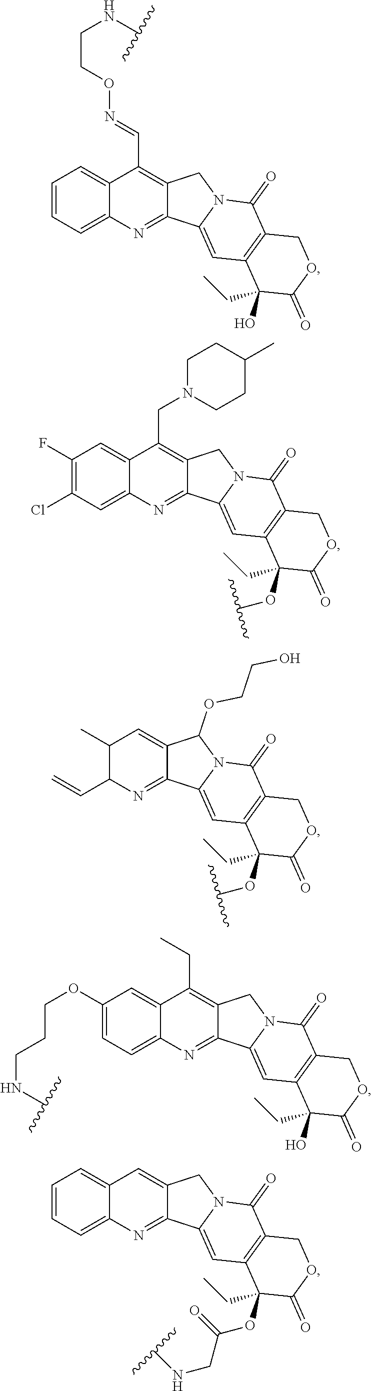

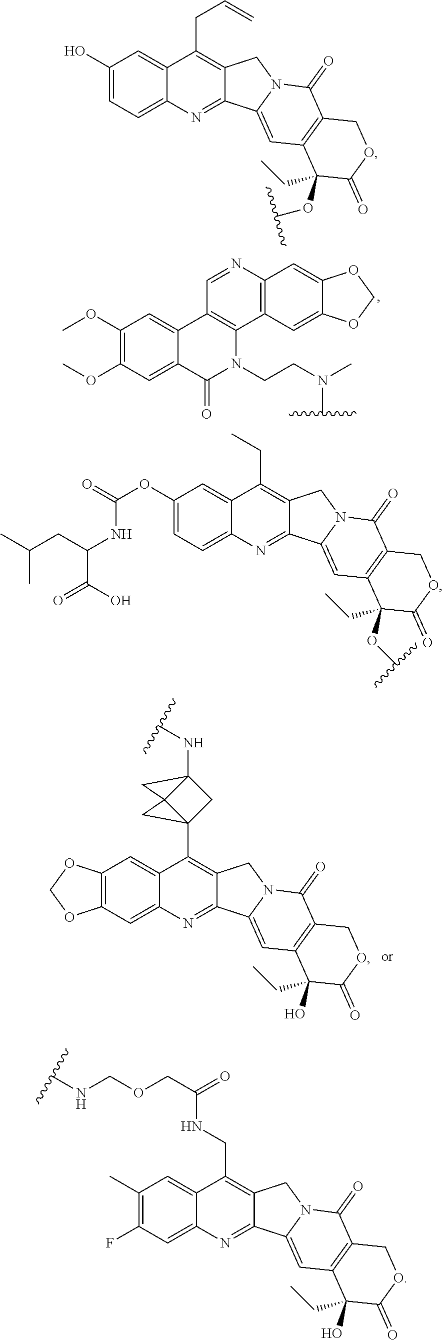

In one embodiment (Id), the disclosure provides an antibody-drug conjugate having formula (I):

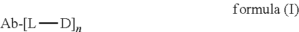

wherein in formula (I):
Ab is an antibody or antibody-binding fragment;
L is a linker;
D is a drug moiety; and
n is an integer from 1 to 20.
   The linker has the following formula:

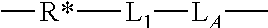

wherein L_{A} is a linker, L₁ is a linking moiety, and R* is a reactive moiety or a targeting agent.
The linker L_{A} has the formula:

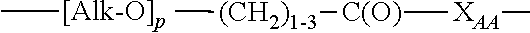

wherein
Alk designates C₂-C₄-alkylene,
X_{AA} is a dipeptide,
p is an integer from 0 to 50.
   R* is a reactive moiety selected from a succinimide, triazole, an amide, and a thioether.
   L₁ is —[CH₂]₀₋₁₂—C(O)NH—.
   The drug moiety is selected from:

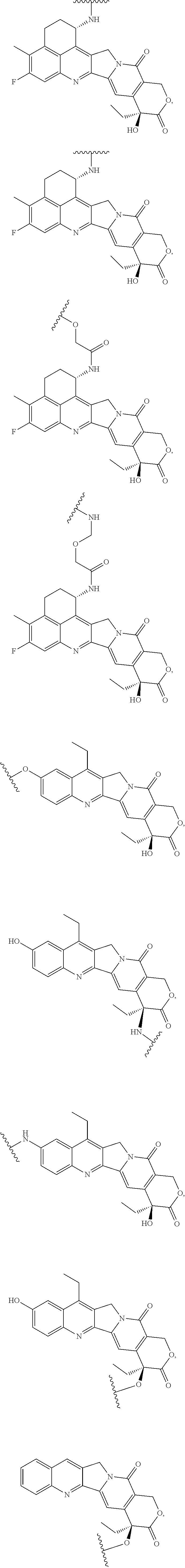

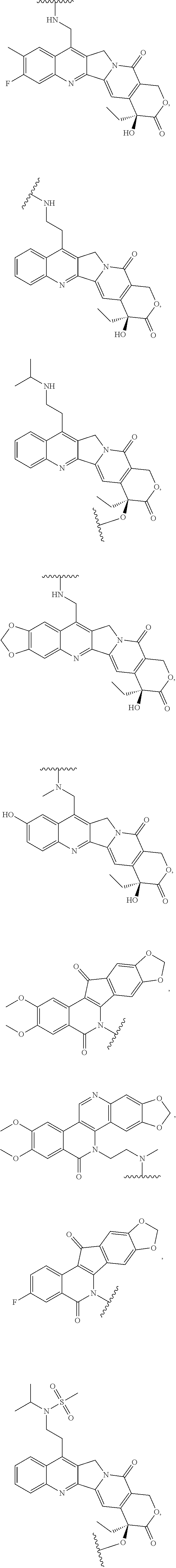

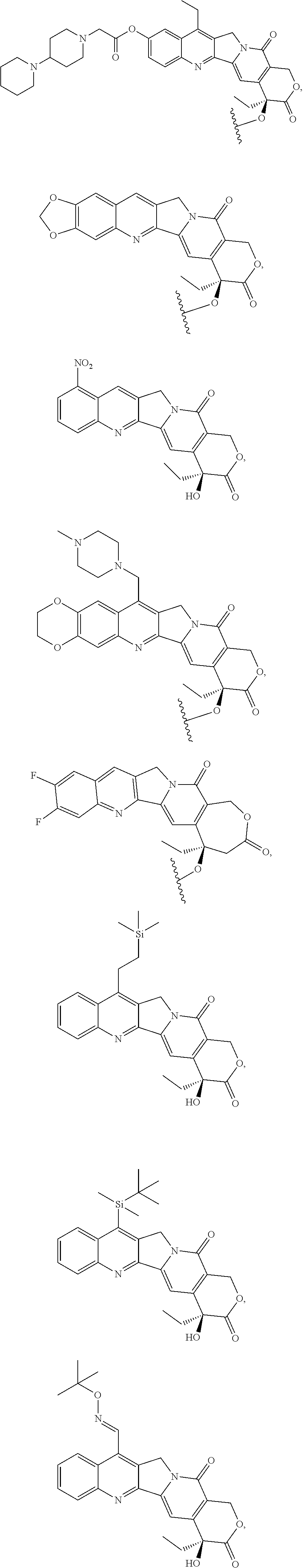

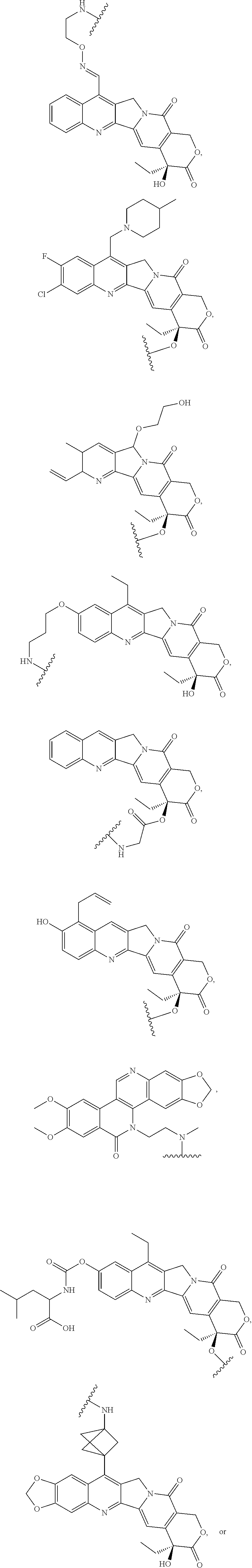

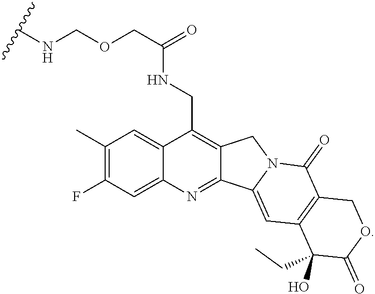 [SMILES: *NCOCC(=O)NCc1c2c(nc3cc(F)c(C)cc13)-c1cc3c(c(=O)n1C2)COC(=O)[C@]3(O)CC]

In one embodiment (Ie), the disclosure provides an antibody-drug conjugate having formula (I):

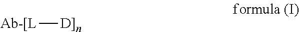

wherein in formula (I):
Ab is an antibody or antibody-binding fragment;
L is a linker;
D is a drug moiety; and
n is an integer from 1 to 20.
   The linker has the following formula:

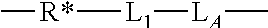

wherein L_{A} is a linker, L₁ is a linking moiety, and R* is a reactive moiety or a targeting agent.
The linker L_{A} has the formula:

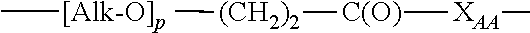

wherein
Alk designates C₂-C₄-alkylene,
X_{AA} is a dipeptide,
p is an integer from 0 to 50.
   R* is a reactive moiety selected from a succinimide, triazole, an amide, and a thioether.
   L₁ is —[CH₂]₀₋₁₂—C(O)NH—.
   The drug moiety is selected from:

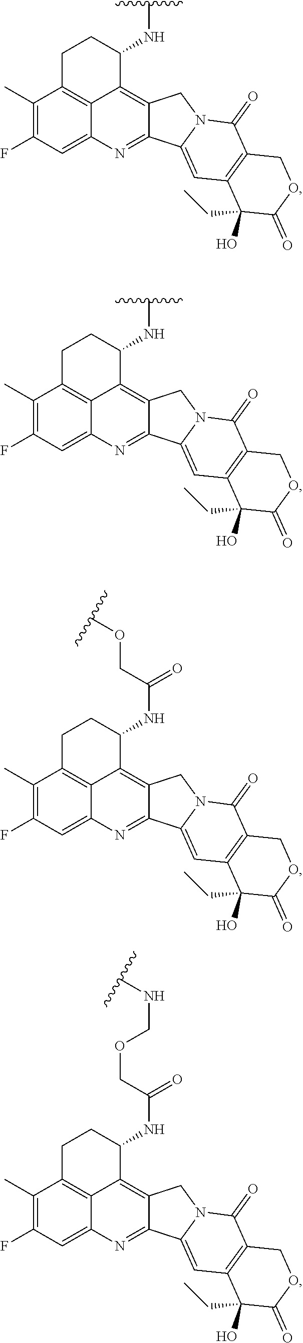 [SMILES: *N[C@H]1CCc2c(C)c(F)cc3nc4c(c1c23)Cn1c-4cc2c(c1=O)COC(=O)[C@]2(O)CC ; *N[C@H]1CCc2c(C)c(F)cc3nc4c(c1c23)Cn1c-4cc2c(c1=O)COC(=O)[C@]2(O)CC ; *OCC(=O)N[C@H]1CCc2c(C)c(F)cc3nc4c(c1c23)Cn1c-4cc2c(c1=O)COC(=O)[C@]2(O)CC ; *NCOCC(=O)N[C@H]1CCc2c(C)c(F)cc3nc4c(c1c23)Cn1c-4cc2c(c1=O)COC(=O)[C@]2(O)CC]

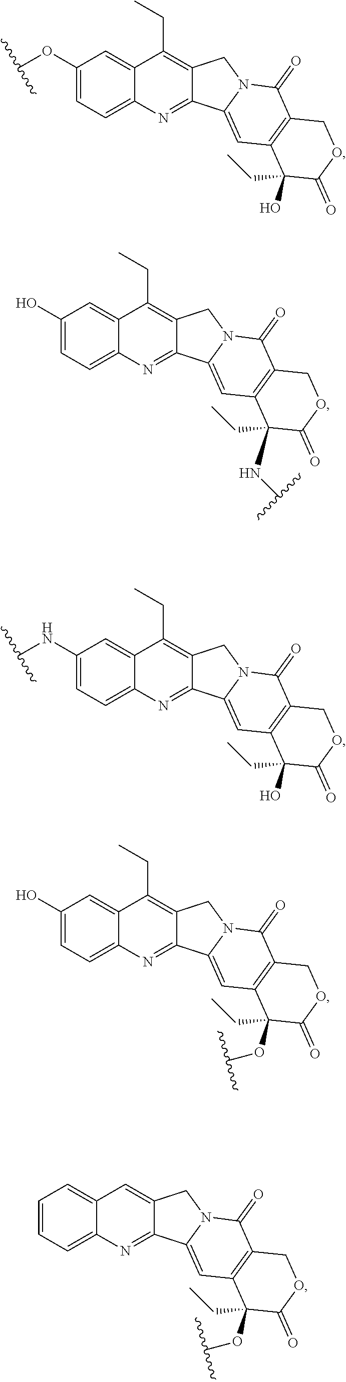 [SMILES: *Oc1ccc2nc3c(c(CC)c2c1)Cn1c-3cc2c(c1=O)COC(=O)[C@]2(O)CC ; *N[C@]1(CC)C(=O)OCc2c1cc1n(c2=O)Cc2c-1nc1ccc(O)cc1c2CC ; *Nc1ccc2nc3c(c(CC)c2c1)Cn1c-3cc2c(c1=O)COC(=O)[C@]2(O)CC ; *O[C@]1(CC)C(=O)OCc2c1cc1n(c2=O)Cc2c-1nc1ccc(O)cc1c2CC ; *O[C@]1(CC)C(=O)OCc2c1cc1n(c2=O)Cc2cc3ccccc3nc2-1]

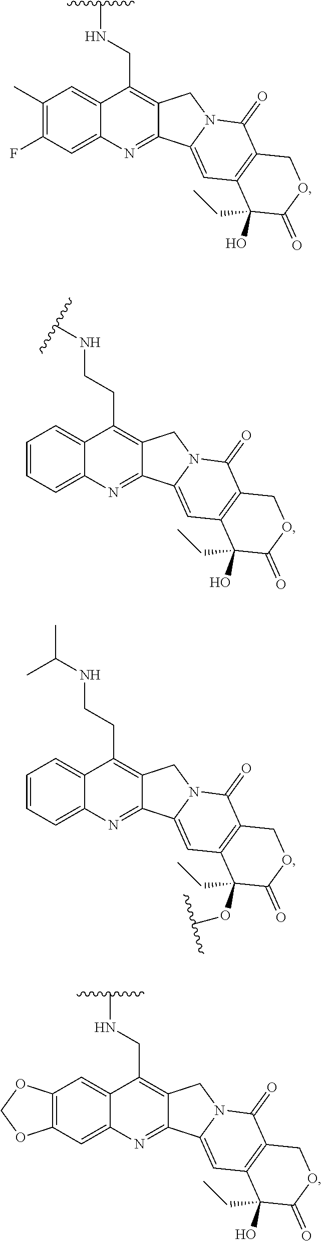 [SMILES: *NCc1c2c(nc3cc(F)c(C)cc13)-c1cc3c(c(=O)n1C2)COC(=O)[C@]3(O)CC ; *NCCc1c2c(nc3ccccc13)-c1cc3c(c(=O)n1C2)COC(=O)[C@]3(O)CC ; *O[C@]1(CC)C(=O)OCc2c1cc1n(c2=O)Cc2c-1nc1ccccc1c2CCNC(C)C ; *NCc1c2c(nc3cc4c(cc13)OCO4)-c1cc3c(c(=O)n1C2)COC(=O)[C@]3(O)CC]

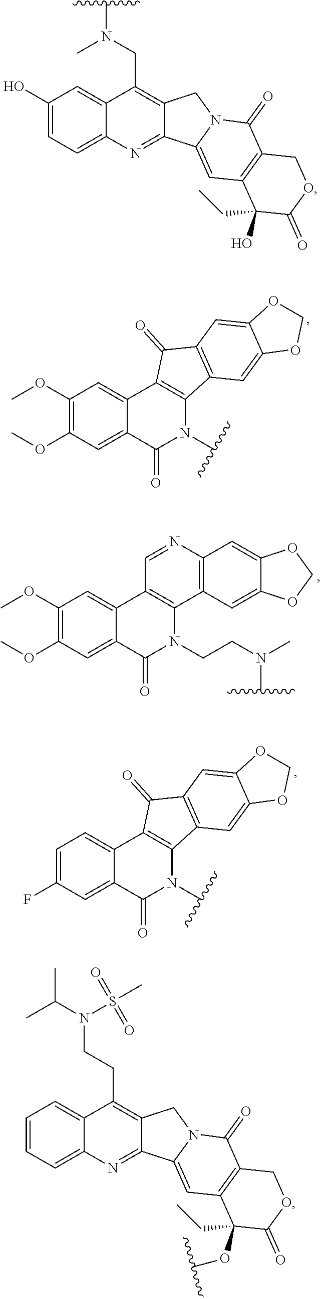 [SMILES: *N(C)Cc1c2c(nc3ccc(O)cc13)-c1cc3c(c(=O)n1C2)COC(=O)[C@]3(O)CC ; *n1c2c(c3cc(OC)c(OC)cc3c1=O)C(=O)c1cc3c(cc1-2)OCO3 ; *N(C)CCn1c(=O)c2cc(OC)c(OC)cc2c2cnc3cc4c(cc3c21)OCO4 ; *n1c2c(c3ccc(F)cc3c1=O)C(=O)c1cc3c(cc1-2)OCO3 ; *O[C@]1(CC)C(=O)OCc2c1cc1n(c2=O)Cc2c-1nc1ccccc1c2CCN(C(C)C)S(C)(=O)=O]

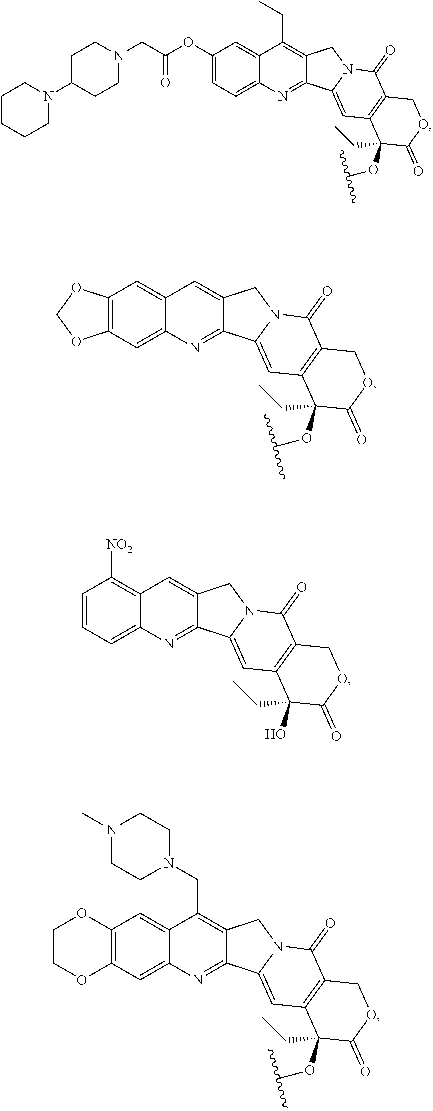 [SMILES: *O[C@]1(CC)C(=O)OCc2c1cc1n(c2=O)Cc2c-1nc1ccc(OC(=O)CN3CCC(N4CCCCC4)CC3)cc1c2CC ; *O[C@]1(CC)C(=O)OCc2c1cc1n(c2=O)Cc2cc3cc4c(cc3nc2-1)OCO4 ; CC[C@@]1(O)C(=O)OCc2c1cc1n(c2=O)Cc2cc3c([N+](=O)[O-])cccc3nc2-1 ; *O[C@]1(CC)C(=O)OCc2c1cc1n(c2=O)Cc2c-1nc1cc3c(cc1c2CN1CCN(C)CC1)OCCO3]

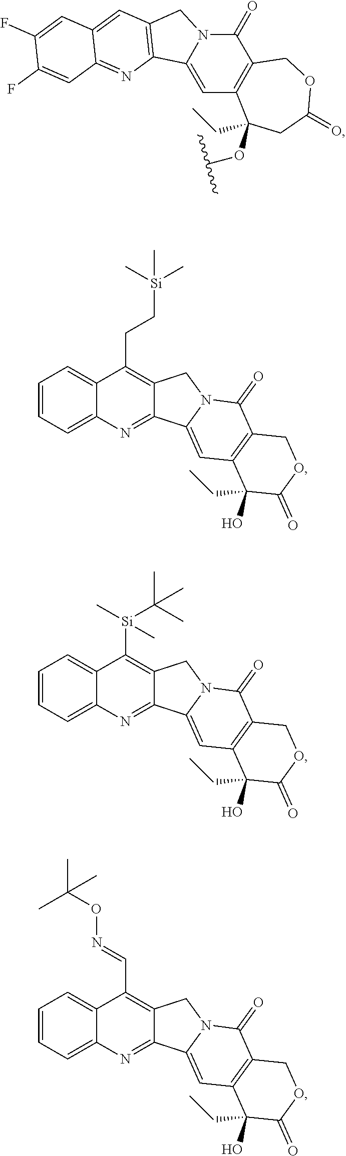 [SMILES: *O[C@]1(CC)CC(=O)OCc2c1cc1n(c2=O)Cc2cc3cc(F)c(F)cc3nc2-1 ; CC[C@@]1(O)C(=O)OCc2c1cc1n(c2=O)Cc2c-1nc1ccccc1c2CC[Si](C)(C)C ; CC[C@@]1(O)C(=O)OCc2c1cc1n(c2=O)Cc2c-1nc1ccccc1c2[Si](C)(C)C(C)(C)C ; CC[C@@]1(O)C(=O)OCc2c1cc1n(c2=O)Cc2c-1nc1ccccc1c2/C=N/OC(C)(C)C]

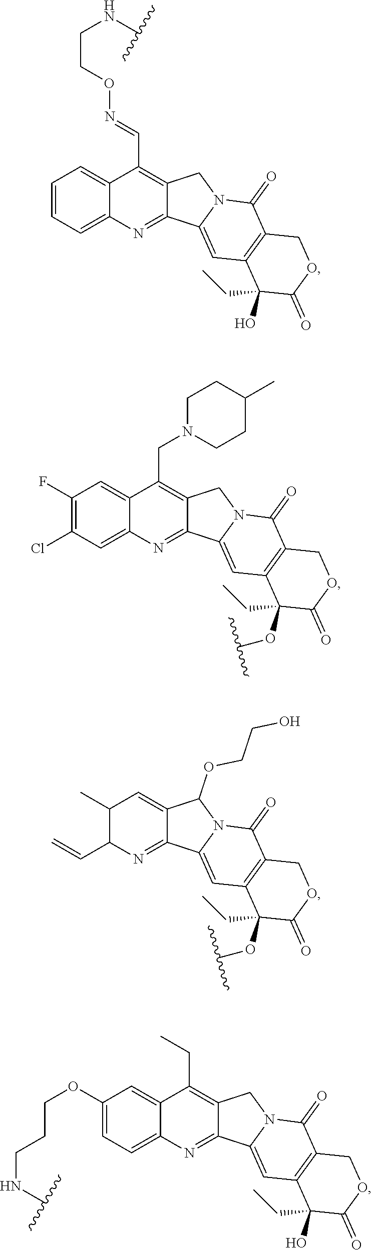 [SMILES: *NCCO/N=C/c1c2c(nc3ccccc13)-c1cc3c(c(=O)n1C2)COC(=O)[C@]3(O)CC ; *O[C@]1(CC)C(=O)OCc2c1cc1n(c2=O)Cc2c-1nc1cc(Cl)c(F)cc1c2CN1CCC(C)CC1 ; *O[C@]1(CC)C(=O)OCc2c1cc1n(c2=O)C(OCCO)C2=CC(C)C(C=C)N=C21 ; *NCCCOc1ccc2nc3c(c(CC)c2c1)Cn1c-3cc2c(c1=O)COC(=O)[C@]2(O)CC]

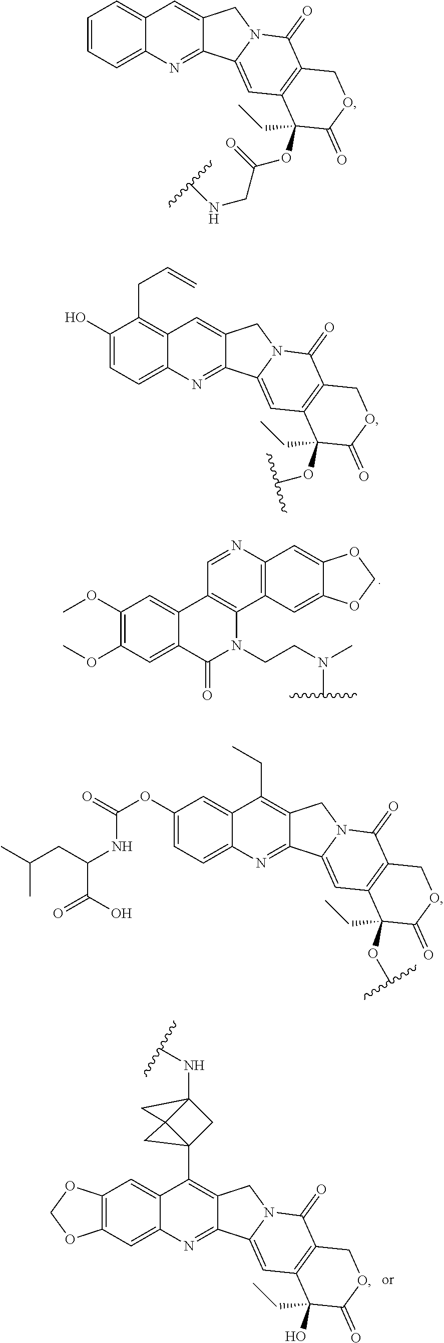 [SMILES: *NCC(=O)O[C@]1(CC)C(=O)OCc2c1cc1n(c2=O)Cc2cc3ccccc3nc2-1 ; *O[C@]1(CC)C(=O)OCc2c1cc1n(c2=O)Cc2cc3c(CC=C)c(O)ccc3nc2-1 ; *N(C)CCn1c(=O)c2cc(OC)c(OC)cc2c2cnc3cc4c(cc3c21)OCO4 ; *O[C@]1(CC)C(=O)OCc2c1cc1n(c2=O)Cc2c-1nc1ccc(OC(=O)NC(CC(C)C)C(=O)O)cc1c2CC ; *NC12CC3(c4c5c(nc6cc7c(cc46)OCO7)-c4cc6c(c(=O)n4C5)COC(=O)[C@]6(O)CC)CC13C2]

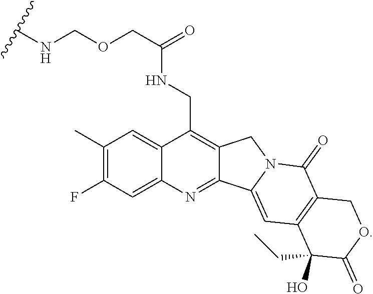 [SMILES: *NCOCC(=O)NCc1c2c(nc3cc(F)c(C)cc13)-c1cc3c(c(=O)n1C2)COC(=O)[C@]3(O)CC]

In one embodiment (II), the disclosure provides an antibody-drug conjugate having formula (I):

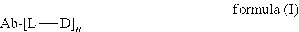

wherein in formula (I):
Ab is an antibody or antibody-binding fragment;
L is a linker;
D is a drug moiety; and
n is an integer from 1 to 20.
   Ab is an antibody or binding fragment thereof that specifically binds CUB Domain-Containing Protein-1 (CDCP1).
   The linker has the following formula:

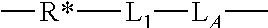

wherein L_{A} is a linker, L₁ is a linking moiety, and R* is a reactive moiety or a targeting agent.
The linker L_{A} has the formula:

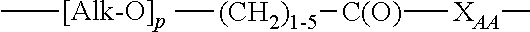

wherein
Alk designates C₂-C₄-alkylene,
X_{AA} is a dipeptide,
p is an integer from 0 to 50.
   R* is a reactive moiety selected from a succinimide, a triazole, an amide, and a thioether.
   Further, R* is or comprises optionally a bisulfone, optionally the bisulfone is or comprises

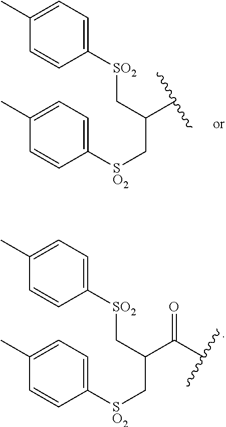 [SMILES: *C(CS(=O)(=O)c1ccc(C)cc1)CS(=O)(=O)c1ccc(C)cc1 ; *C(=O)C(CS(=O)(=O)c1ccc(C)cc1)CS(=O)(=O)c1ccc(C)cc1]

L₁ is —[CH₂]₀₋₁₂—C(O)NH—.
The drug moiety is selected from exatecan having the formula:

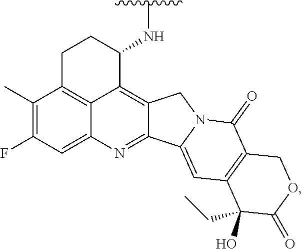 [SMILES: *N[C@H]1CCc2c(C)c(F)cc3nc4c(c1c23)Cn1c-4cc2c(c1=O)COC(=O)[C@]2(O)CC]

Dxd, Sn-38, monomethyl auristatin E (MMAE), and a pyrridinobenzodiazepine (PDD).

In one embodiment (IIa), the disclosure provides an antibody-drug conjugate having formula (I):

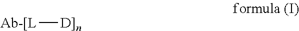

wherein in formula (I):
Ab is an antibody or antibody-binding fragment;
L is a linker;
D is a drug moiety; and
n is an integer from 1 to 20.
   Ab is an antibody or binding fragment thereof that specifically binds CUB Domain-Containing Protein-1 (CDCP1).
   The linker has the following formula:

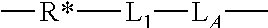

wherein L_{A} is a linker, L₁ is a linking moiety, and R* is a reactive moiety or a targeting agent.
The linker L_{A} has the formula:

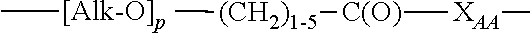

wherein
Alk designates C₂-C₄-alkylene,
X_{AA} is a dipeptide,
p is an integer from 0 to 50.
   R* is a reactive moiety selected from a succinimide, a triazole, an amide, and a thioether.
   Further, R* is or comprises optionally a bisulfone, optionally the bisulfone is or comprises

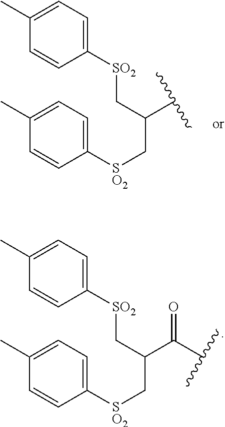 [SMILES: *C(CS(=O)(=O)c1ccc(C)cc1)CS(=O)(=O)c1ccc(C)cc1 ; *C(=O)C(CS(=O)(=O)c1ccc(C)cc1)CS(=O)(=O)c1ccc(C)cc1]

L₁ is —[CH₂]₀₋₁₂—C(O)NH—.
The drug moiety is selected from:

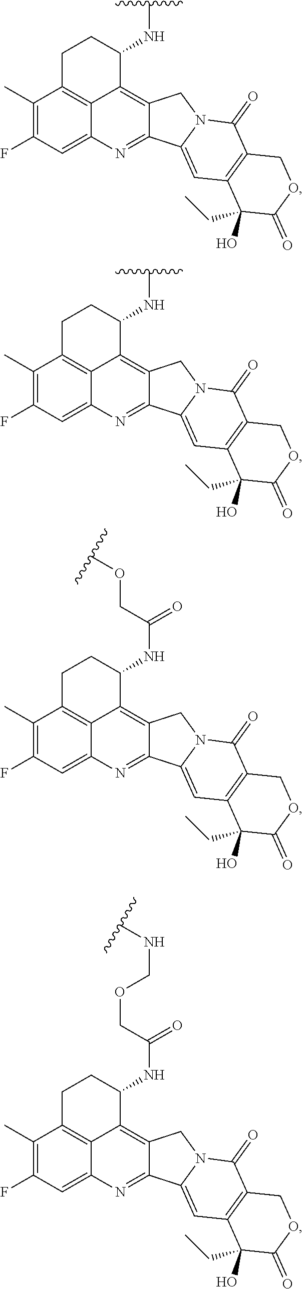 [SMILES: *N[C@H]1CCc2c(C)c(F)cc3nc4c(c1c23)Cn1c-4cc2c(c1=O)COC(=O)[C@]2(O)CC ; *N[C@H]1CCc2c(C)c(F)cc3nc4c(c1c23)Cn1c-4cc2c(c1=O)COC(=O)[C@]2(O)CC ; *OCC(=O)N[C@H]1CCc2c(C)c(F)cc3nc4c(c1c23)Cn1c-4cc2c(c1=O)COC(=O)[C@]2(O)CC ; *NCOCC(=O)N[C@H]1CCc2c(C)c(F)cc3nc4c(c1c23)Cn1c-4cc2c(c1=O)COC(=O)[C@]2(O)CC]

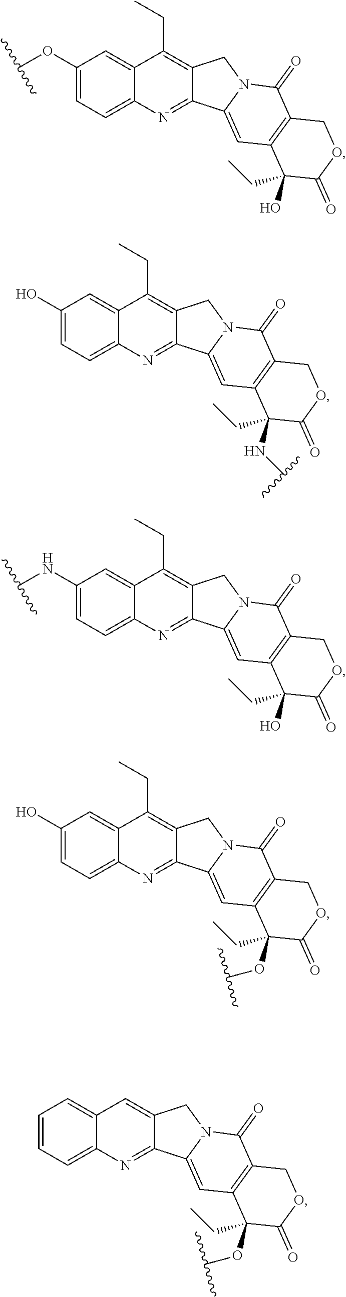 [SMILES: *Oc1ccc2nc3c(c(CC)c2c1)Cn1c-3cc2c(c1=O)COC(=O)[C@]2(O)CC ; *N[C@]1(CC)C(=O)OCc2c1cc1n(c2=O)Cc2c-1nc1ccc(O)cc1c2CC ; *Nc1ccc2nc3c(c(CC)c2c1)Cn1c-3cc2c(c1=O)COC(=O)[C@]2(O)CC ; *O[C@]1(CC)C(=O)OCc2c1cc1n(c2=O)Cc2c-1nc1ccc(O)cc1c2CC ; *O[C@]1(CC)C(=O)OCc2c1cc1n(c2=O)Cc2cc3ccccc3nc2-1]

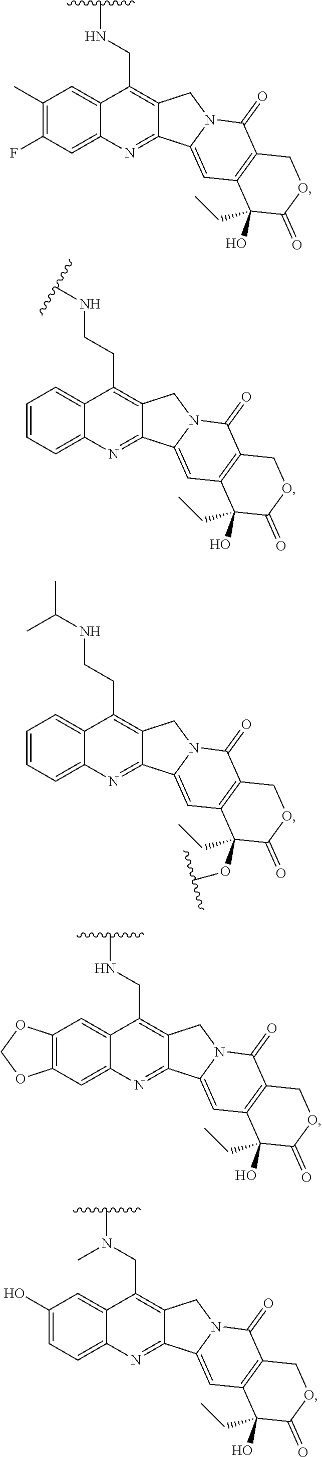 [SMILES: *NCc1c2c(nc3cc(F)c(C)cc13)-c1cc3c(c(=O)n1C2)COC(=O)[C@]3(O)CC ; *NCCc1c2c(nc3ccccc13)-c1cc3c(c(=O)n1C2)COC(=O)[C@]3(O)CC ; *O[C@]1(CC)C(=O)OCc2c1cc1n(c2=O)Cc2c-1nc1ccccc1c2CCNC(C)C ; *NCc1c2c(nc3cc4c(cc13)OCO4)-c1cc3c(c(=O)n1C2)COC(=O)[C@]3(O)CC ; *N(C)Cc1c2c(nc3ccc(O)cc13)-c1cc3c(c(=O)n1C2)COC(=O)[C@]3(O)CC]

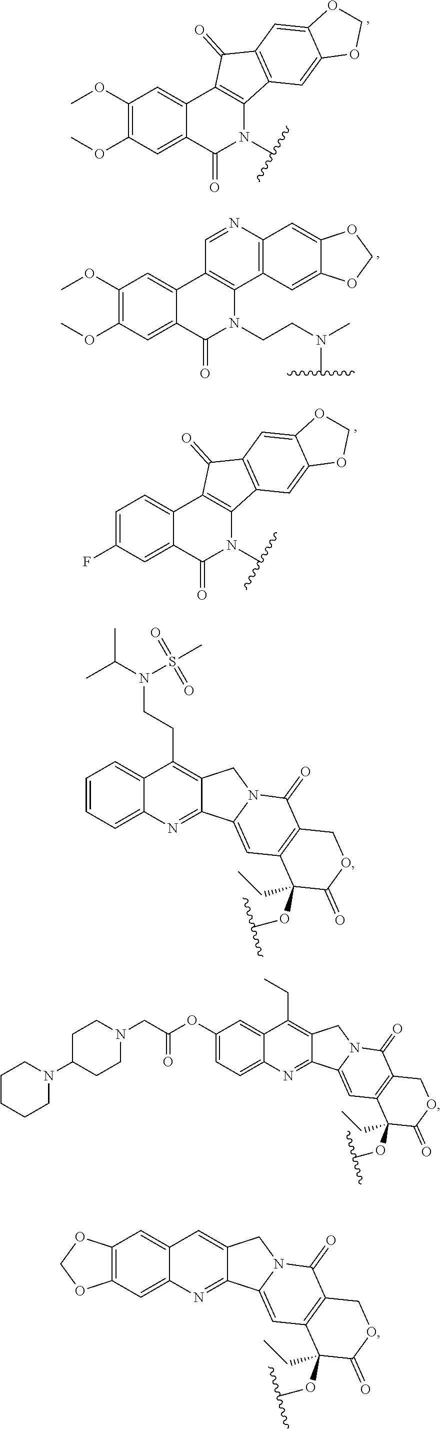

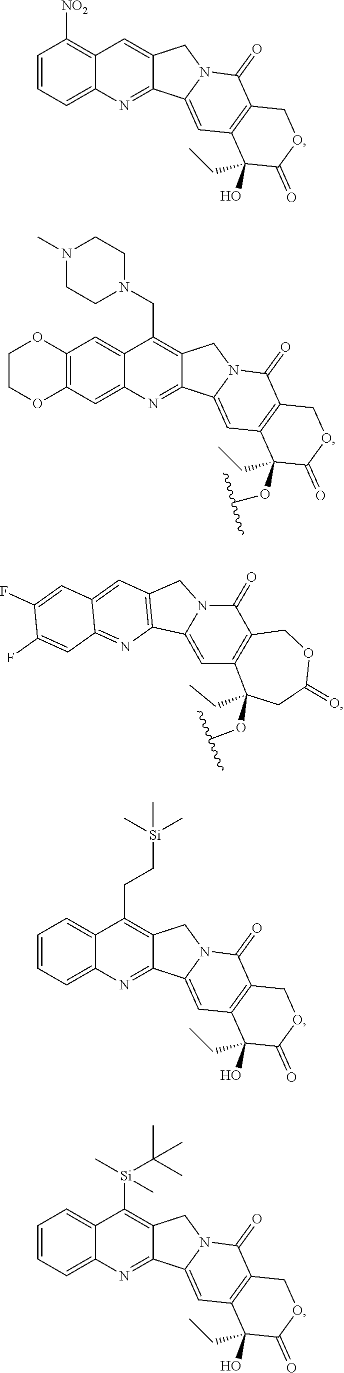 [SMILES: CC[C@@]1(O)C(=O)OCc2c1cc1n(c2=O)Cc2cc3c([N+](=O)[O-])cccc3nc2-1 ; *O[C@]1(CC)C(=O)OCc2c1cc1n(c2=O)Cc2c-1nc1cc3c(cc1c2CN1CCN(C)CC1)OCCO3 ; *O[C@]1(CC)CC(=O)OCc2c1cc1n(c2=O)Cc2cc3cc(F)c(F)cc3nc2-1 ; CC[C@@]1(O)C(=O)OCc2c1cc1n(c2=O)Cc2c-1nc1ccccc1c2CC[Si](C)(C)C ; CC[C@@]1(O)C(=O)OCc2c1cc1n(c2=O)Cc2c-1nc1ccccc1c2[Si](C)(C)C(C)(C)C]

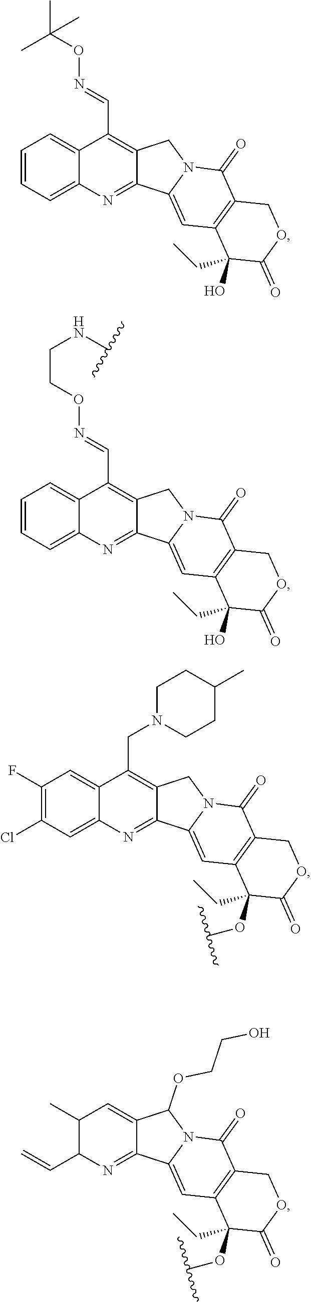

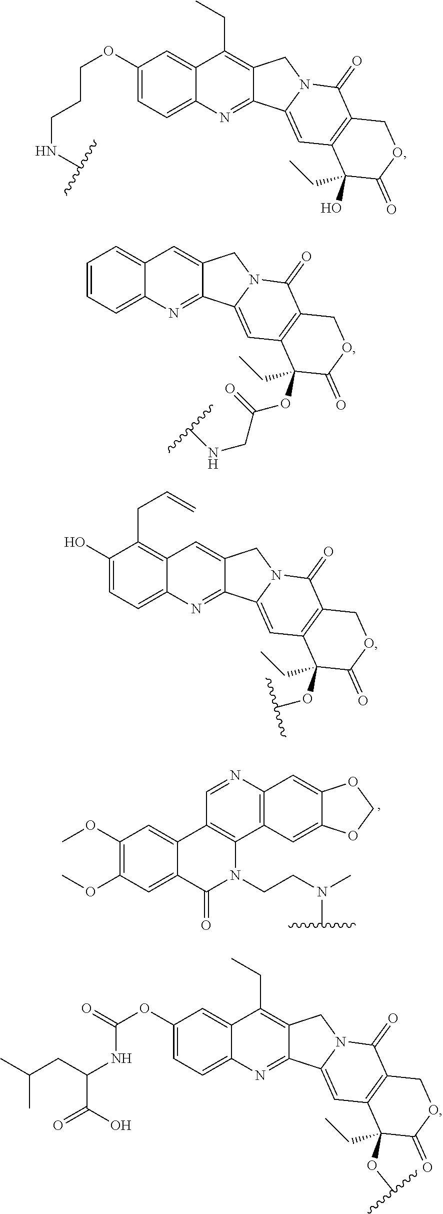

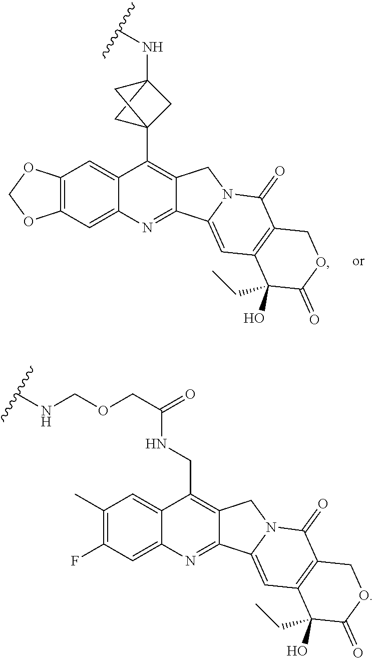 [SMILES: *NC12CC(c3c4c(nc5cc6c(cc35)OCO6)-c3cc5c(c(=O)n3C4)COC(=O)[C@]5(O)CC)(C1)C2 ; "or" ; *NCOCC(=O)NCc1c2c(nc3cc(F)c(C)cc13)-c1cc3c(c(=O)n1C2)COC(=O)[C@]3(O)CC]

In one embodiment (IIb), the disclosure provides an antibody-drug conjugate having formula (I):

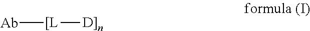

wherein in formula (I):
Ab is an antibody or antibody-binding fragment;
L is a linker;
D is a drug moiety; and
n is an integer from 1 to 20.
   Ab is an antibody or binding fragment thereof that specifically binds CUB Domain-Containing Protein-1 (CDCP1).
   The linker has the following formula:

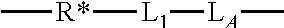

wherein L_{A} is a linker, L₁ is a linking moiety, and R* is a reactive moiety or a targeting agent.
The linker L_{A} has the formula:

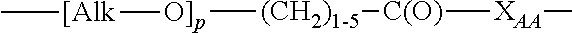

wherein
Alk designates C₂-C₄-alkylene,
X_{AA} is a dipeptide,
p is an integer from 0 to 50.
   R* is a reactive moiety selected from a succinimide, a triazole, an amide, and a thioether.
   Further, R* is or comprises optionally a bisulfone, optionally the bisulfone is or comprises

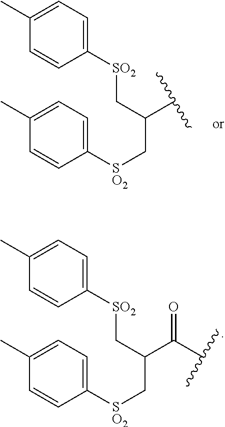 [SMILES: *C(CS(=O)(=O)c1ccc(C)cc1)CS(=O)(=O)c1ccc(C)cc1 ; *C(=O)C(CS(=O)(=O)c1ccc(C)cc1)CS(=O)(=O)c1ccc(C)cc1]

L₁ is —[CH₂]₀₋₁₂—C(O)NH—.
The drug moiety is selected from:

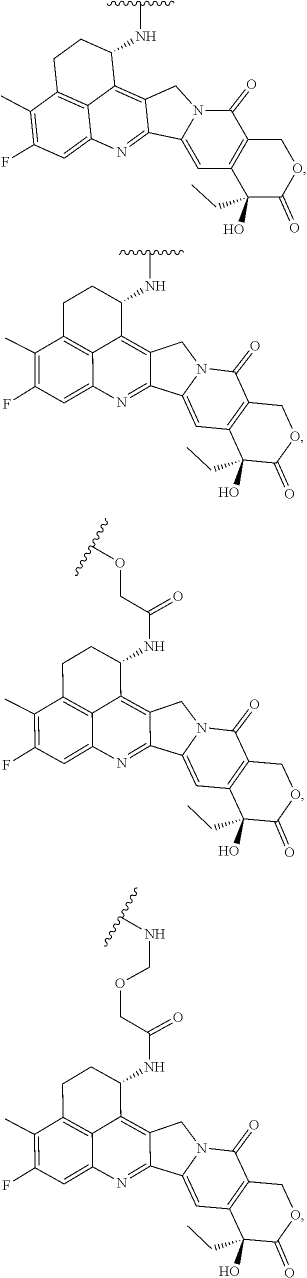 [SMILES: *N[C@H]1CCc2c(C)c(F)cc3nc4c(c1c23)Cn1c-4cc2c(c1=O)COC(=O)[C@]2(O)CC ; *N[C@H]1CCc2c(C)c(F)cc3nc4c(c1c23)Cn1c-4cc2c(c1=O)COC(=O)[C@]2(O)CC ; *OCC(=O)N[C@H]1CCc2c(C)c(F)cc3nc4c(c1c23)Cn1c-4cc2c(c1=O)COC(=O)[C@]2(O)CC ; *NCOCC(=O)N[C@H]1CCc2c(C)c(F)cc3nc4c(c1c23)Cn1c-4cc2c(c1=O)COC(=O)[C@]2(O)CC]

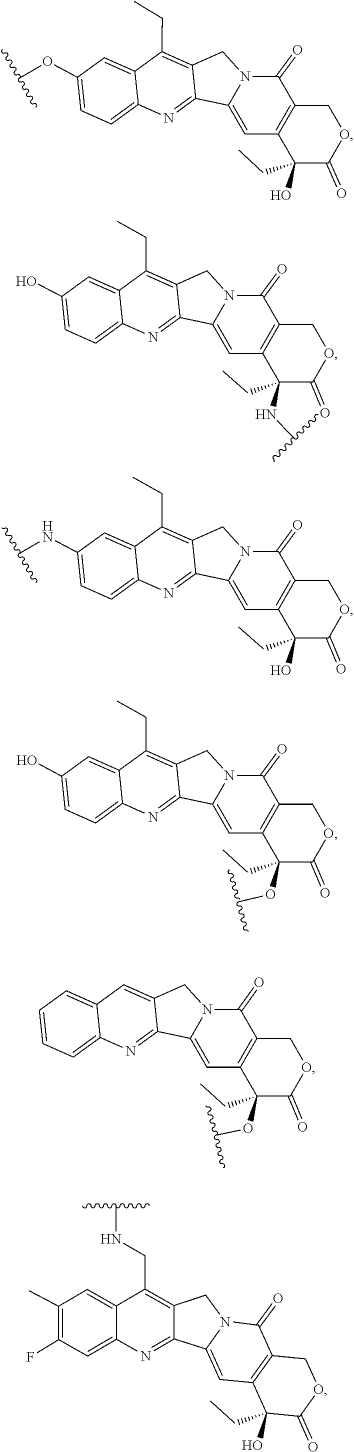 [SMILES: *Oc1ccc2nc3c(c(CC)c2c1)Cn1c-3cc2c(c1=O)COC(=O)[C@]2(O)CC ; *N[C@]1(CC)C(=O)OCc2c1cc1n(c2=O)Cc2c-1nc1ccc(O)cc1c2CC ; *Nc1ccc2nc3c(c(CC)c2c1)Cn1c-3cc2c(c1=O)COC(=O)[C@]2(O)CC ; *O[C@]1(CC)C(=O)OCc2c1cc1n(c2=O)Cc2c-1nc1ccc(O)cc1c2CC ; *O[C@]1(CC)C(=O)OCc2c1cc1n(c2=O)Cc2cc3ccccc3nc2-1 ; *NCc1c2c(nc3cc(F)c(C)cc13)-c1cc3c(c(=O)n1C2)COC(=O)[C@]3(O)CC]

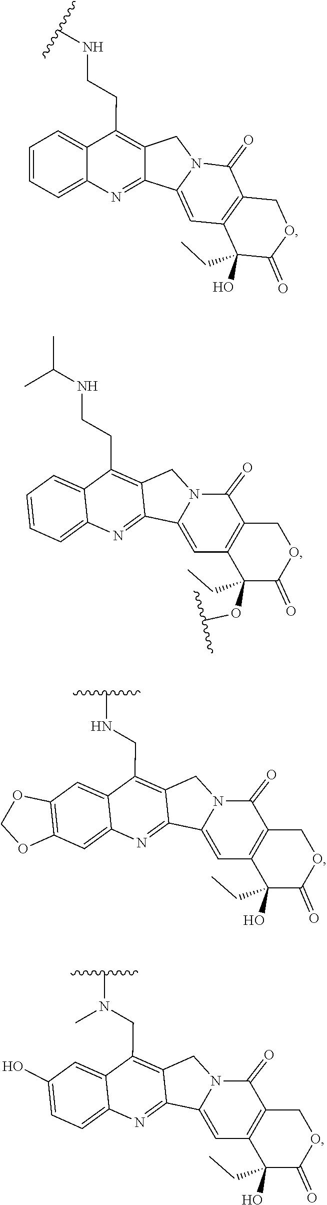 [SMILES: *NCCc1c2c(nc3ccccc13)-c1cc3c(c(=O)n1C2)COC(=O)[C@]3(O)CC ; *O[C@]1(CC)C(=O)OCc2c1cc1n(c2=O)Cc2c-1nc1ccccc1c2CCNC(C)C ; *NCc1c2c(nc3cc4c(cc13)OCO4)-c1cc3c(c(=O)n1C2)COC(=O)[C@]3(O)CC ; *N(C)Cc1c2c(nc3ccc(O)cc13)-c1cc3c(c(=O)n1C2)COC(=O)[C@]3(O)CC]

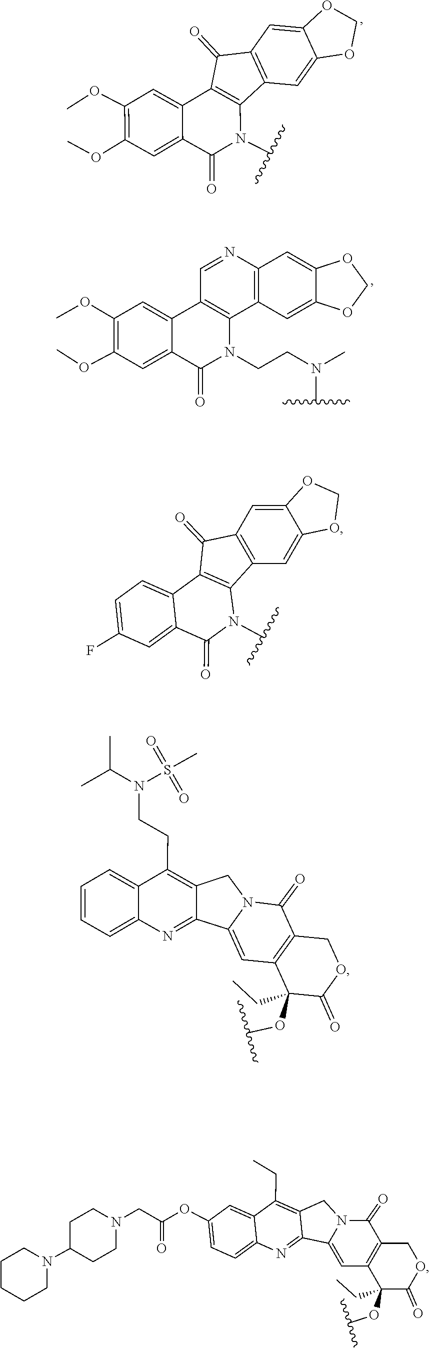 [SMILES: *n1c2c(c3cc(OC)c(OC)cc3c1=O)C(=O)c1cc3c(cc1-2)OCO3 ; *N(C)CCn1c(=O)c2cc(OC)c(OC)cc2c2cnc3cc4c(cc3c21)OCO4 ; *n1c2c(c3ccc(F)cc3c1=O)C(=O)c1cc3c(cc1-2)OCO3 ; *O[C@]1(CC)C(=O)OCc2c1cc1n(c2=O)Cc2c-1nc1ccccc1c2CCN(C(C)C)S(C)(=O)=O ; *O[C@]1(CC)C(=O)OCc2c1cc1n(c2=O)Cc2c-1nc1ccc(OC(=O)CN3CCC(N4CCCCC4)CC3)cc1c2CC]

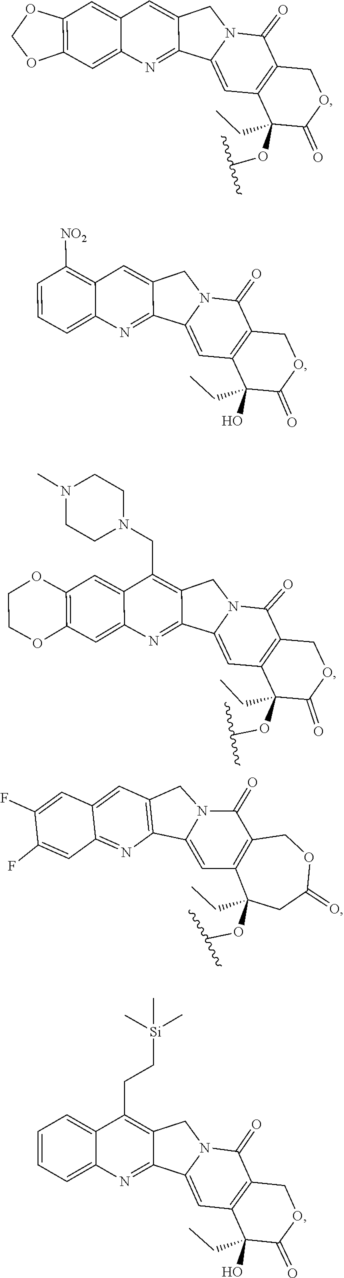 [SMILES: *O[C@]1(CC)C(=O)OCc2c1cc1n(c2=O)Cc2cc3cc4c(cc3nc2-1)OCO4 ; CC[C@@]1(O)C(=O)OCc2c1cc1n(c2=O)Cc2cc3c([N+](=O)[O-])cccc3nc2-1 ; *O[C@]1(CC)C(=O)OCc2c1cc1n(c2=O)Cc2c-1nc1cc3c(cc1c2CN1CCN(C)CC1)OCCO3 ; *O[C@]1(CC)CC(=O)OCc2c1cc1n(c2=O)Cc2cc3cc(F)c(F)cc3nc2-1 ; CC[C@@]1(O)C(=O)OCc2c1cc1n(c2=O)Cc2c-1nc1ccccc1c2CC[Si](C)(C)C]

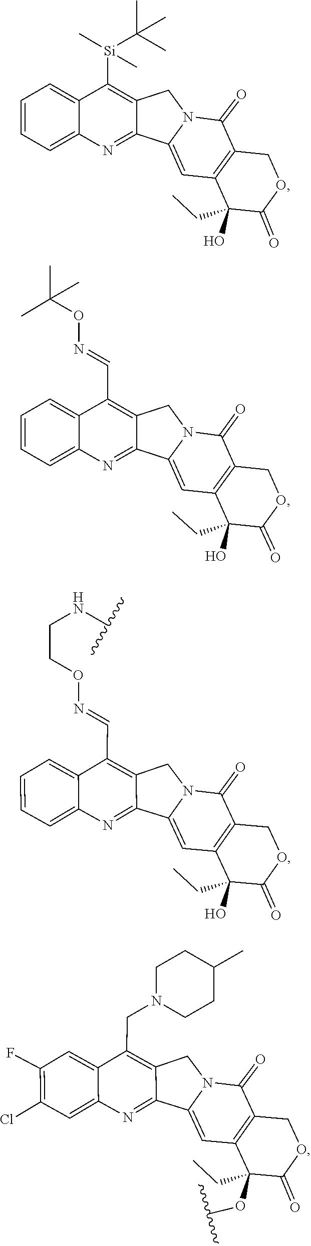

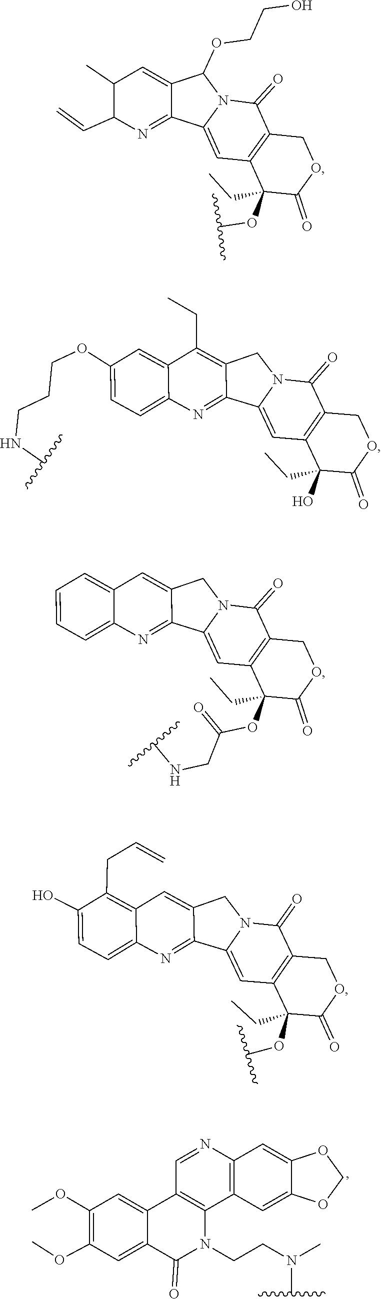 [SMILES: *O[C@]1(CC)C(=O)OCc2c1cc1n(c2=O)C(OCCO)C2=CC(C)C(C=C)N=C21 ; *NCCCOc1ccc2nc3c(c(CC)c2c1)Cn1c-3cc2c(c1=O)COC(=O)[C@]2(O)CC ; *NCC(=O)O[C@]1(CC)C(=O)OCc2c1cc1n(c2=O)Cc2cc3ccccc3nc2-1 ; *O[C@]1(CC)C(=O)OCc2c1cc1n(c2=O)Cc2cc3c(CC=C)c(O)ccc3nc2-1 ; *N(C)CCn1c(=O)c2cc(OC)c(OC)cc2c2cnc3cc4c(cc3c21)OCO4]

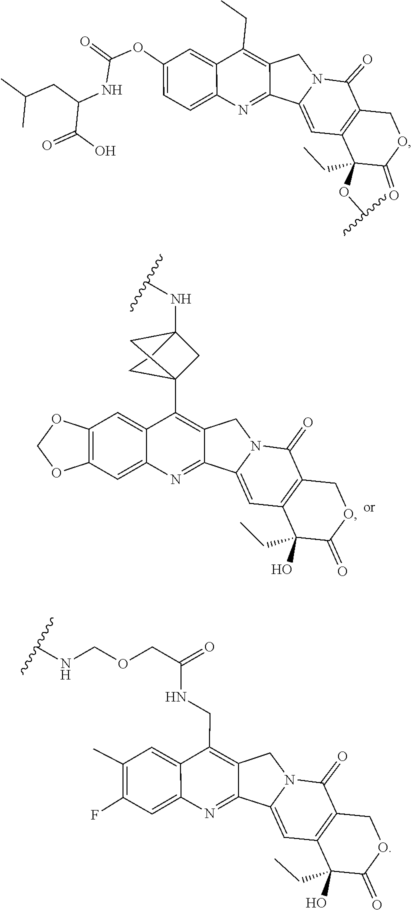 [SMILES: *O[C@]1(CC)C(=O)OCc2c1cc1n(c2=O)Cc2c-1nc1ccc(OC(=O)NC(CC(C)C)C(=O)O)cc1c2CC ; *NC12CCC(c3c4c(nc5cc6c(cc35)OCO6)-c3cc5c(c(=O)n3C4)COC(=O)[C@]5(O)CC)(C1)C2 ; *NCOCC(=O)NCc1c2c(nc3cc(F)c(C)cc13)-c1cc3c(c(=O)n1C2)COC(=O)[C@]3(O)CC]

In one embodiment (III), the disclosure provides an antibody-drug conjugate having formula (I):

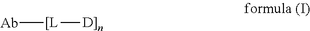

wherein in formula (I):
Ab is an antibody or antibody-binding fragment;
L is a linker;
D is a drug moiety; and
n is an integer from 1 to 20.
   The antibody or binding fragment thereof comprises
(i) a heavy chain variable region (VH) that comprises:
   (a) a VH complementarity determining region one (CDRH1) comprising and/or consisting of the amino acid sequence of SEQ ID NO: 2,
   (b) a VH complementarity determining region two (CDRH2) comprising and/or consisting of the amino acid sequence of SEQ ID NO: 3, and
   (c) a VH complementarity determining region three (CDRH3) comprising and/or consisting of the amino acid sequence of SEQ ID NO: 4,
and (ii) a light chain variable region (VL) that comprises:
   (a) a VL complementarity determining region one (CDRL1) comprising and/or consisting of the amino acid sequence of SEQ ID NO: 6,
   (b) a VL complementarity determining region two (CDRL2) comprising and/or consisting of the amino acid sequence of SEQ ID NO: 7, and
   (c) a VL complementarity determining region three (CDRL3) comprising and/or consisting of the amino acid sequence of SEQ ID NO:8.
      The linker has the following formula:

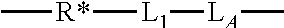

wherein L_{A} is a linker, L₁ is a linking moiety, and R* is a reactive moiety or a targeting agent.
The linker L_{A} has the formula:

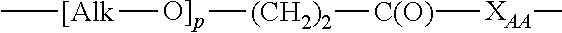

wherein
Alk designates C₂-C₄-alkylene,
X_{AA} is a dipeptide,
p is an integer from 0 to 50.
   R* is a reactive moiety selected from a succinimide, a triazole, an amide, and a thioether.
   L₁ is —[CH₂]₀₋₁₂—C(O)NH—.
   The drug moiety is selected from exatecan having the formula:

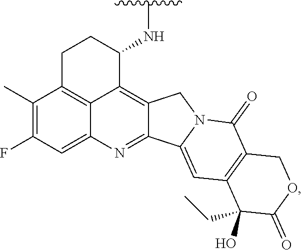 [SMILES: *N[C@H]1CCc2c(C)c(F)cc3nc4c(c1c23)Cn1c-4cc2c(c1=O)COC(=O)[C@]2(O)CC]

Dxd, Sn-38, monomethyl auristatin E (MMAE), and a pyrridinobenzodiazepine (PDD).

In one embodiment (IV), the disclosure provides an antibody-drug conjugate having formula (I):

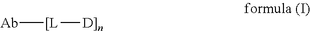

wherein in formula (I):
Ab is an antibody or antibody-binding fragment;
L is a linker;
D is a drug moiety; and
n is an integer from 1 to 20.
   The antibody or binding fragment thereof comprises
(i) a heavy chain variable region (VH) that comprises:
   (a) a VH complementarity determining region one (CDRH1) comprising and/or consisting of the amino acid sequence of SEQ ID NO: 12,
   (b) a VH complementarity determining region two (CDRH2) comprising and/or consisting of the amino acid sequence of SEQ ID NO: 13, and
   (c) a VH complementarity determining region three (CDRH3) comprising and/or consisting of the amino acid sequence of SEQ ID NO: 14,
and (ii) a light chain variable region (VL) that comprises:
   (a) a VL complementarity determining region one (CDRL1) comprising and/or consisting of the amino acid sequence of SEQ ID NO: 6,
   (b) a VL complementarity determining region two (CDRL2) comprising and/or consisting of the amino acid sequence of SEQ ID NO: 7, and
   (c) a VL complementarity determining region three (CDRL3) comprising and/or consisting of the amino acid sequence of SEQ ID NO:8.
      The linker has the following formula:

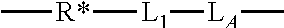

wherein L_{A} is a linker, L₁ is a linking moiety, and R* is a reactive moiety or a targeting agent.
The linker L_{A} has the formula:

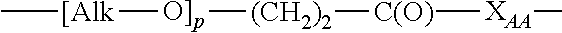

wherein
Alk designates C₂-C₄-alkylene,
X_{AA} is a dipeptide,
p is an integer from 0 to 50.
   R* is a reactive moiety selected from a succinimide, a triazole, an amide, and a thioether.
   L₁ is —[CH₂]₀₋₁₂—C(O)NH—.
   The drug moiety is selected from exatecan having the formula:

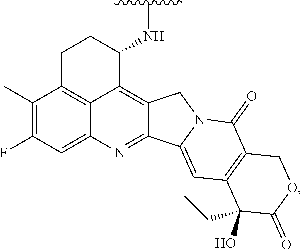 [SMILES: *N[C@H]1CCc2c(C)c(F)cc3nc4c(c1c23)Cn1c-4cc2c(c1=O)COC(=O)[C@]2(O)CC]

Dxd, Sn-38, monomethyl auristatin E (MMAE), and a pyrridinobenzodiazepine (PDD).

In one embodiment (V), the disclosure provides an antibody-drug conjugate having formula (I):

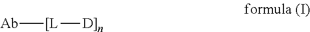

wherein in formula (I):
Ab is an antibody or antibody-binding fragment;
L is a linker;
D is a drug moiety; and
n is an integer from 1 to 20.
   The antibody or binding fragment thereof Ab comprises a VH that comprises and/or consists of an amino acid sequence of SEQ ID NO: 1 and/or comprises a VL that comprises and/or consists of an amino acid sequence of SEQ ID NO: 5.
   The linker has the following formula:

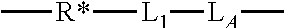

wherein L_{A} is a linker, L₁ is a linking moiety, and R* is a reactive moiety or a targeting agent.
The linker L_{A} has the formula:

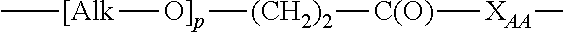

wherein
Alk designates C₂-C₄-alkylene,
X_{AA} is a dipeptide,
p is an integer from 0 to 50.
   R* is a reactive moiety selected from a succinimide, a triazole, an amide, and a thioether.
   L₁ is —[CH₂]₀₋₁₂—C(O)NH—.
   The drug moiety is selected from exatecan having the formula:

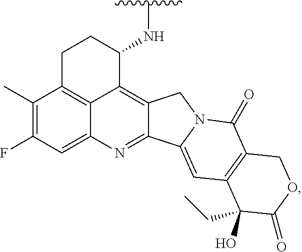 [SMILES: *N[C@H]1CCc2c(C)c(F)cc3nc4c(c1c23)Cn1c-4cc2c(c1=O)COC(=O)[C@]2(O)CC]

Dxd, Sn-38, monomethyl auristatin E (MMAE), and a pyrridinobenzodiazepine (PDD).

In one embodiment (VI), the disclosure provides an antibody-drug conjugate having formula (I):

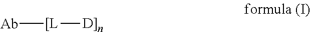

wherein in formula (I):
Ab is an antibody or antibody-binding fragment;
L is a linker;
D is a drug moiety; and
n is an integer from 1 to 20.
   The antibody or binding fragment thereof Ab comprises a heavy chain that comprises an amino acid sequence of SEQ ID NO: 1 and/or comprises a light chain that comprises an amino acid sequence of SEQ ID NO: 5.
   The linker has the following formula:

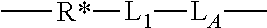

wherein L_{A} is a linker, L₁ is a linking moiety, and R* is a reactive moiety or a targeting agent.
The linker L_{A} has the formula:

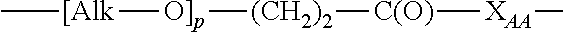

wherein
Alk designates C₂-C₄-alkylene,
X_{AA} is a dipeptide,
p is an integer from 0 to 50.
   R* is a reactive moiety selected from a succinimide, a triazole, an amide, and a thioether.
   L₁ is —[CH₂]₀₋₁₂—C(O)NH—.
   The drug moiety is selected from exatecan having the formula:

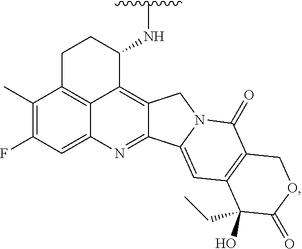 [SMILES: *N[C@H]1CCc2c(C)c(F)cc3nc4c(c1c23)Cn1c-4cc2c(c1=O)COC(=O)[C@]2(O)CC]

Dxd, Sn-38, monomethyl auristatin E (MMAE), and a pyrridinobenzodiazepine (PDD).

In one embodiment (VII), the disclosure provides an antibody-drug conjugate having formula (I):

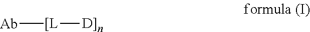

wherein in formula (I):
Ab is an antibody or antibody-binding fragment;
L is a linker;
D is a drug moiety; and
n is an integer from 1 to 20.
   The linker has the following formula:

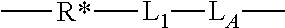

wherein L_{A} is a linker, L₁ is a linking moiety, and R* is a reactive moiety or a targeting agent.
The linker L_{A} has the formula:

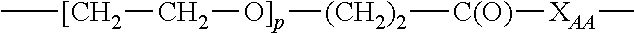

wherein
X_{AA} is a nonpolar dipeptide,
p is an integer from 5 to 10, e.g. 5, 6, 7, 8, 9, or 10.
   R* is a reactive moiety selected from a succinimide, a triazole, an amide, and a thioether.
   L₁ is —[CH₂]₀₋₁₂—C(O)NH—.
   The drug moiety is selected from exatecan having the formula:

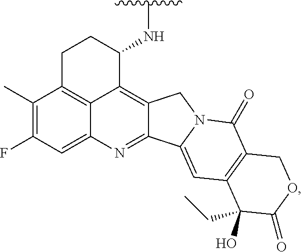 [SMILES: *N[C@H]1CCc2c(C)c(F)cc3nc4c(c1c23)Cn1c-4cc2c(c1=O)COC(=O)[C@]2(O)CC]

Dxd, Sn-38, monomethyl auristatin E (MMAE), and a pyrridinobenzodiazepine (PDD).

In one embodiment (VIII), the disclosure provides an antibody-drug conjugate having formula (I):

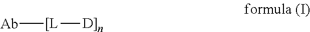

wherein in formula (I):
Ab is an antibody or antibody-binding fragment;
L is a linker;
D is a drug moiety; and
n is an integer from 1 to 20.
   Ab is an antibody or binding fragment thereof that specifically binds CUB Domain-Containing Protein-1 (CDCP1).
   The linker has the following formula:

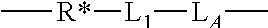

wherein L_{A} is a linker, L₁ is a linking moiety, and R* is a reactive moiety or a targeting agent.
The linker L_{A} has the formula:

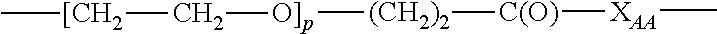

wherein
X_{AA} is a nonpolar dipeptide,
p is an integer from 5 to 10, e.g. 5, 6, 7, 8, 9, or 10.
   R* is a reactive moiety selected from a succinimide, a triazole, an amide, and a thioether.
   L₁ is —[CH₂]₀₋₁₂—C(O)NH—.
   The drug moiety is selected from exatecan having the formula:

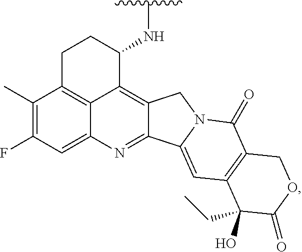 [SMILES: *N[C@H]1CCc2c(C)c(F)cc3nc4c(c1c23)Cn1c-4cc2c(c1=O)COC(=O)[C@]2(O)CC]

Dxd, Sn-38, monomethyl auristatin E (MMAE), and a pyrridinobenzodiazepine (PDD).

In one embodiment (IX), the disclosure provides an antibody-drug conjugate having formula (I):

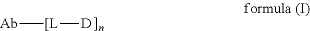

wherein in formula (I):
Ab is an antibody or antibody-binding fragment;
L is a linker;
D is a drug moiety; and
n is an integer from 1 to 20.
   The antibody or binding fragment thereof comprises
(i) a heavy chain variable region (VH) that comprises:
   (a) a VH complementarity determining region one (CDRH1) comprising and/or consisting of the amino acid sequence of SEQ ID NO: 2,
   (b) a VH complementarity determining region two (CDRH2) comprising and/or consisting of the amino acid sequence of SEQ ID NO: 3, and
   (c) a VH complementarity determining region three (CDRH3) comprising and/or consisting of the amino acid sequence of SEQ ID NO: 4,
and (ii) a light chain variable region (VL) that comprises:
   (a) a VL complementarity determining region one (CDRL1) comprising and/or consisting of the amino acid sequence of SEQ ID NO: 6,
   (b) a VL complementarity determining region two (CDRL2) comprising and/or consisting of the amino acid sequence of SEQ ID NO: 7, and
   (c) a VL complementarity determining region three (CDRL3) comprising and/or consisting of the amino acid sequence of SEQ ID NO:8.
      The linker has the following formula:

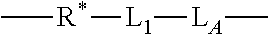

wherein L_{A} is a linker, L₁ is a linking moiety, and R* is a reactive moiety or a targeting agent.
The linker L_{A} has the formula:

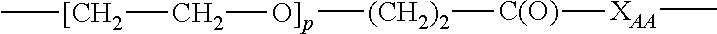

wherein
X_{AA} is a nonpolar dipeptide,
p is an integer from 5 to 10, e.g. 5, 6, 7, 8, 9, or 10.
   R* is a reactive moiety selected from a succinimide, a triazole, an amide, and a thioether.
   L₁ is —[CH₂]₀₋₁₂—C(O)NH—.
   The drug moiety is selected from exatecan having the formula:

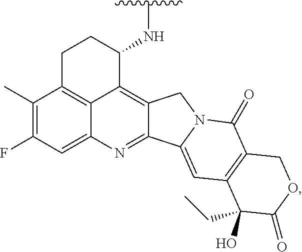 [SMILES: *N[C@H]1CCc2c(C)c(F)cc3nc4c(c1c23)Cn1c-4cc2c(c1=O)COC(=O)[C@]2(O)CC]

Dxd, Sn-38, monomethyl auristatin E (MMAE), and a pyrridinobenzodiazepine (PDD).

In one embodiment (X), the disclosure provides an antibody-drug conjugate having formula (I):

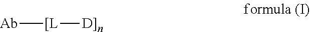

wherein in formula (I):
Ab is an antibody or antibody-binding fragment;
L is a linker;
D is a drug moiety; and
n is an integer from 1 to 20.
   The antibody or binding fragment thereof comprises
(i) a heavy chain variable region (VH) that comprises:
   (a) a VH complementarity determining region one (CDRH1) comprising and/or consisting of the amino acid sequence of SEQ ID NO: 12,
   (b) a VH complementarity determining region two (CDRH2) comprising and/or consisting of the amino acid sequence of SEQ ID NO: 13, and
   (c) a VH complementarity determining region three (CDRH3) comprising and/or consisting of the amino acid sequence of SEQ ID NO: 14,
and (ii) a light chain variable region (VL) that comprises:
   (a) a VL complementarity determining region one (CDRL1) comprising and/or consisting of the amino acid sequence of SEQ ID NO: 6,
   (b) a VL complementarity determining region two (CDRL2) comprising and/or consisting of the amino acid sequence of SEQ ID NO: 7, and
   (c) a VL complementarity determining region three (CDRL3) comprising and/or consisting of the amino acid sequence of SEQ ID NO:8.
      The linker has the following formula:

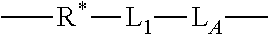

wherein L_{A} is a linker, L₁ is a linking moiety, and R* is a reactive moiety or a targeting agent.
The linker L_{A} has the formula:

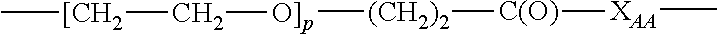

wherein
X_{AA} is a nonpolar dipeptide,
p is an integer from 5 to 10, e.g. 5, 6, 7, 8, 9, or 10.
   R* is a reactive moiety selected from a succinimide, a triazole, an amide, and a thioether.
   L₁ is —[CH₂]₀₋₁₂—C(O)NH—.
   The drug moiety is selected from exatecan having the formula:

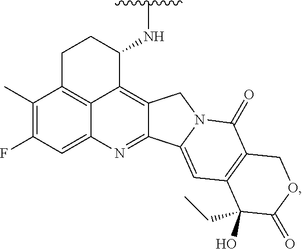 [SMILES: *N[C@H]1CCc2c(C)c(F)cc3nc4c(c1c23)Cn1c-4cc2c(c1=O)COC(=O)[C@]2(O)CC]

Dxd, Sn-38, monomethyl auristatin E (MMAE), and a pyrridinobenzodiazepine (PDD).

In one embodiment (XI), the disclosure provides an antibody-drug conjugate having formula (I):

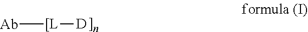

wherein in formula (I):
Ab is an antibody or antibody-binding fragment;
L is a linker;
D is a drug moiety; and
n is an integer from 1 to 20.
   The antibody or binding fragment thereof Ab comprises a VH that comprises and/or consists of an amino acid sequence of SEQ ID NO: 1 and/or comprises a VL that comprises and/or consists of an amino acid sequence of SEQ ID NO: 5.
   The linker has the following formula:

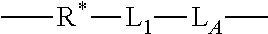

wherein L_{A} is a linker, L₁ is a linking moiety, and R* is a reactive moiety or a targeting agent.
The linker L_{A} has the formula:

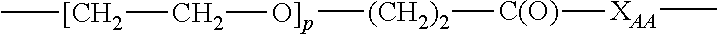

wherein
X_{AA} is a nonpolar dipeptide,
p is an integer from 5 to 10, e.g. 5, 6, 7, 8, 9, or 10.
   R* is a reactive moiety selected from a succinimide, a triazole, an amide, and a thioether.
   L₁ is —[CH₂]₀₋₁₂—C(O)NH—.
   The drug moiety is selected from exatecan having the formula:

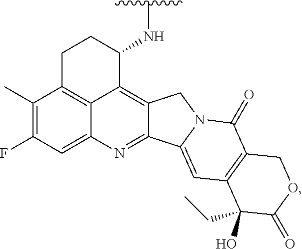 [SMILES: *N[C@H]1CCc2c(C)c(F)cc3nc4c(c1c23)Cn1c-4cc2c(c1=O)COC(=O)[C@]2(O)CC]

Dxd, Sn-38, monomethyl auristatin E (MMAE), and a pyrridinobenzodiazepine (PDD).

In one embodiment (XII), the disclosure provides an antibody-drug conjugate having formula (I):

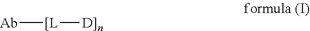

wherein in formula (I):
Ab is an antibody or antibody-binding fragment;
L is a linker;
D is a drug moiety; and
n is an integer from 1 to 20.
   The antibody or binding fragment thereof Ab comprises a heavy chain that comprises an amino acid sequence of SEQ ID NO: 1 and/or comprises a light chain that comprises an amino acid sequence of SEQ ID NO: 5.
   The linker has the following formula:

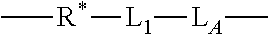

wherein L_{A} is a linker, L₁ is a linking moiety, and R* is a reactive moiety or a targeting agent.
The linker L_{A} has the formula:

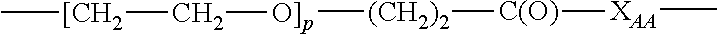

wherein
X_{AA} is a nonpolar dipeptide,
p is an integer from 5 to 10, e.g. 5, 6, 7, 8, 9, or 10.
   R* is a reactive moiety selected from a succinimide, a triazole, an amide, and a thioether.
   L₁ is —[CH₂]₀₋₁₂—C(O)NH—.
   The drug moiety is selected from exatecan having the formula:

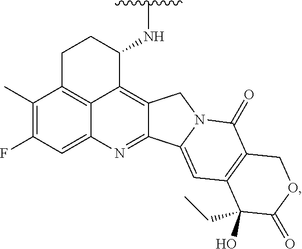 [SMILES: *N[C@H]1CCc2c(C)c(F)cc3nc4c(c1c23)Cn1c-4cc2c(c1=O)COC(=O)[C@]2(O)CC]

Dxd, Sn-38, monomethyl auristatin E (MMAE), and a pyrridinobenzodiazepine (PDD).

In one embodiment (XIII), the disclosure provides an antibody-drug conjugate having formula (I):

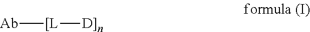

wherein in formula (I):
Ab is an antibody or antibody-binding fragment;
L is a linker;
D is a drug moiety; and
n is an integer from 1 to 20.
   The linker has the following formula:

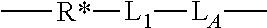

wherein L_{A} is a linker, L₁ is a linking moiety, and R* is a reactive moiety or a targeting agent.
The linker L_{A} has the formula:

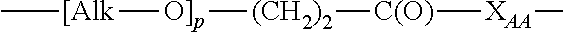

wherein
Alk designates C₂-C₄-alkylene,
X_{AA} is a dipeptide,
p is an integer from 0 to 50.
   R* is succinimide.
   L₁ is —[CH₂]₁₋₃—C(O)NH—.
   The drug moiety is selected from exatecan having the formula:

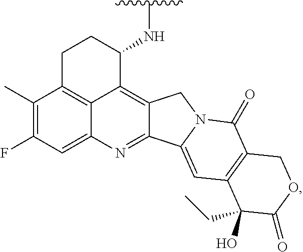 [SMILES: *N[C@H]1CCc2c(C)c(F)cc3nc4c(c1c23)Cn1c-4cc2c(c1=O)COC(=O)[C@]2(O)CC]

Dxd, Sn-38, monomethyl auristatin E (MMAE), and a pyrridinobenzodiazepine (PDD).

In one embodiment (XIV), the disclosure provides an antibody-drug conjugate having formula (I):

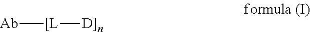

wherein in formula (I):
Ab is an antibody or antibody-binding fragment;
L is a linker;
D is a drug moiety; and
n is an integer from 1 to 20.
   Ab is an antibody or binding fragment thereof that specifically binds CUB Domain-Containing Protein-1 (CDCP1).
   The linker has the following formula:

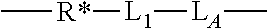

wherein L_{A} is a linker, L₁ is a linking moiety, and R* is a reactive moiety or a targeting agent.
The linker L_{A} has the formula:

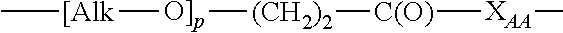

wherein
Alk designates C₂-C₄-alkylene,
X_{AA} is a dipeptide,
p is an integer from 0 to 50.
   R* is succinimide.
   L₁ is —[CH₂]₁₋₃—C(O)NH—.
   The drug moiety is selected from exatecan having the formula:

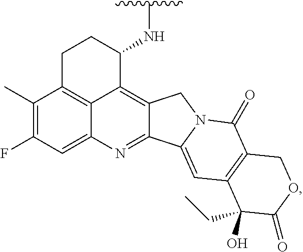 [SMILES: *N[C@H]1CCc2c(C)c(F)cc3nc4c(c1c23)Cn1c-4cc2c(c1=O)COC(=O)[C@]2(O)CC]

Dxd, Sn-38, monomethyl auristatin E (MMAE), and a pyrridinobenzodiazepine (PDD).

In one embodiment (XV), the disclosure provides an antibody-drug conjugate having formula (I):

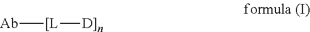

wherein in formula (I):
Ab is an antibody or antibody-binding fragment;
L is a linker;
D is a drug moiety; and
n is an integer from 1 to 20.
   The antibody or binding fragment thereof comprises
(i) a heavy chain variable region (VH) that comprises:
   (a) a VH complementarity determining region one (CDRH1) comprising and/or consisting of the amino acid sequence of SEQ ID NO: 2,
   (b) a VH complementarity determining region two (CDRH2) comprising and/or consisting of the amino acid sequence of SEQ ID NO: 3, and
   (c) a VH complementarity determining region three (CDRH3) comprising and/or consisting of the amino acid sequence of SEQ ID NO: 4,
and (ii) a light chain variable region (VL) that comprises:
   (a) a VL complementarity determining region one (CDRL1) comprising and/or consisting of the amino acid sequence of SEQ ID NO: 6,
   (b) a VL complementarity determining region two (CDRL2) comprising and/or consisting of the amino acid sequence of SEQ ID NO: 7, and
   (c) a VL complementarity determining region three (CDRL3) comprising and/or consisting of the amino acid sequence of SEQ ID NO:8.
      The linker has the following formula:

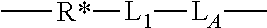

wherein L_{A} is a linker, L₁ is a linking moiety, and R* is a reactive moiety or a targeting agent.
The linker L_{A} has the formula:

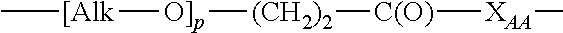

wherein
Alk designates C₂-C₄-alkylene,
X_{AA} is a dipeptide,
p is an integer from 0 to 50.
   R* is succinimide.
   L₁ is —[CH₂]₁₋₃—C(O)NH—.
   The drug moiety is selected from exatecan having the formula:

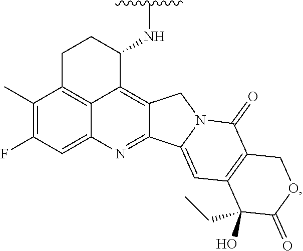 [SMILES: *N[C@H]1CCc2c(C)c(F)cc3nc4c(c1c23)Cn1c-4cc2c(c1=O)COC(=O)[C@]2(O)CC]

Dxd, Sn-38, monomethyl auristatin E (MMAE), and a pyrridinobenzodiazepine (PDD).

In one embodiment (XVI), the disclosure provides an antibody-drug conjugate having formula (I):

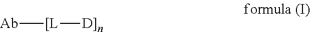

wherein in formula (I):
Ab is an antibody or antibody-binding fragment;
L is a linker;
D is a drug moiety; and
n is an integer from 1 to 20.
   The antibody or binding fragment thereof comprises
(i) a heavy chain variable region (VH) that comprises:
   (a) a VH complementarity determining region one (CDRH1) comprising and/or consisting of the amino acid sequence of SEQ ID NO: 12,
   (b) a VH complementarity determining region two (CDRH2) comprising and/or consisting of the amino acid sequence of SEQ ID NO: 13, and
   (c) a VH complementarity determining region three (CDRH3) comprising and/or consisting of the amino acid sequence of SEQ ID NO: 14,
and (ii) a light chain variable region (VL) that comprises:
   (a) a VL complementarity determining region one (CDRL1) comprising and/or consisting of the amino acid sequence of SEQ ID NO: 6,
   (b) a VL complementarity determining region two (CDRL2) comprising and/or consisting of the amino acid sequence of SEQ ID NO: 7, and
   (c) a VL complementarity determining region three (CDRL3) comprising and/or consisting of the amino acid sequence of SEQ ID NO:8.
      The linker has the following formula:

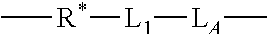

wherein L_{A} is a linker, L₁ is a linking moiety, and R* is a reactive moiety or a targeting agent.
The linker L_{A} has the formula:

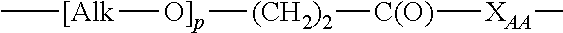

wherein
Alk designates C₂-C₄-alkylene,
X_{AA} is a dipeptide,
p is an integer from 0 to 50.
   R* is succinimide
   L₁ is —[CH₂]₁₋₃—C(O)NH—.
   The drug moiety is selected from exatecan having the formula:

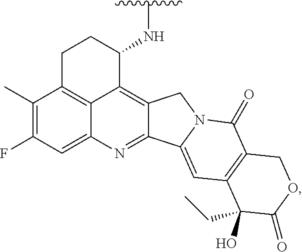 [SMILES: *N[C@H]1CCc2c(C)c(F)cc3nc4c(c1c23)Cn1c-4cc2c(c1=O)COC(=O)[C@]2(O)CC]

Dxd, Sn-38, monomethyl auristatin E (MMAE), and a pyrridinobenzodiazepine (PDD).

In one embodiment (XVII), the disclosure provides an antibody-drug conjugate having formula (I):

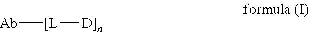

wherein in formula (I):
Ab is an antibody or antibody-binding fragment;
L is a linker;
D is a drug moiety; and
n is an integer from 1 to 20.
   The antibody or binding fragment thereof Ab comprises a VH that comprises and/or consists of an amino acid sequence of SEQ ID NO: 1 and/or comprises a VL that comprises and/or consists of an amino acid sequence of SEQ ID NO: 5.
   The linker has the following formula:

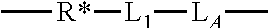

wherein L_{A} is a linker, L₁ is a linking moiety, and R* is a reactive moiety or a targeting agent.
The linker L_{A} has the formula:

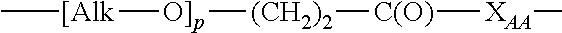

wherein
Alk designates C₂-C₄-alkylene,
X_{AA} is a dipeptide,
p is an integer from 0 to 50.
   R* is succinimide
   L₁ is —[CH₂]₁₋₃—C(O)NH—.
   The drug moiety is selected from exatecan having the formula:

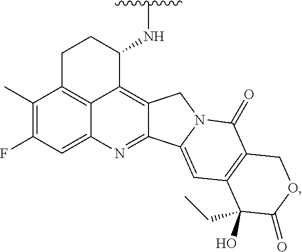 [SMILES: *N[C@H]1CCc2c(C)c(F)cc3nc4c(c1c23)Cn1c-4cc2c(c1=O)COC(=O)[C@]2(O)CC]

Dxd, Sn-38, monomethyl auristatin E (MMAE), and a pyrridinobenzodiazepine (PDD).

In one embodiment (XVIII), the disclosure provides an antibody-drug conjugate having formula (I):

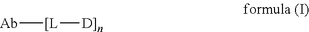

wherein in formula (I):
Ab is an antibody or antibody-binding fragment;
L is a linker;
D is a drug moiety; and
n is an integer from 1 to 20.
   The antibody or binding fragment thereof Ab comprises a heavy chain that comprises an amino acid sequence of SEQ ID NO: 1 and/or comprises a light chain that comprises an amino acid sequence of SEQ ID NO: 5.
   The linker has the following formula:

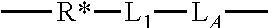

wherein L_{A} is a linker, L₁ is a linking moiety, and R* is a reactive moiety or a targeting agent.
The linker L_{A} has the formula:

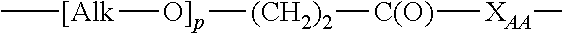

wherein
Alk designates C₂-C₄-alkylene,
X_{AA} is a dipeptide,
p is an integer from 0 to 50.
   R* is succinimide.
   L₁ is —[CH₂]₁₋₃—C(O)NH—.
   The drug moiety is selected from exatecan having the formula:

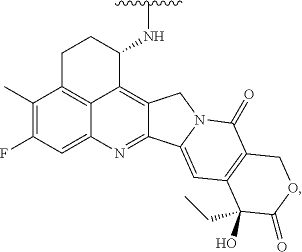 [SMILES: *N[C@H]1CCc2c(C)c(F)cc3nc4c(c1c23)Cn1c-4cc2c(c1=O)COC(=O)[C@]2(O)CC]

Dxd, Sn-38, monomethyl auristatin E (MMAE), and a pyrridinobenzodiazepine (PDD).

In one embodiment (XIX), the disclosure provides an antibody-drug conjugate having formula (I):

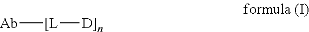

wherein in formula (I):
Ab is an antibody or antibody-binding fragment;
L is a linker;
D is a drug moiety; and
n is an integer from 1 to 20.
   The linker has the following formula:

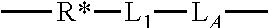

wherein L_{A} is a linker, L₁ is a linking moiety, and R* is a reactive moiety or a targeting agent.
The linker L_{A} has the formula:

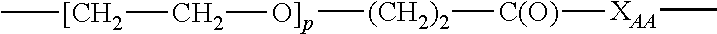

wherein
X_{AA} is a nonpolar dipeptide,
p is an integer from 5 to 10, e.g. 5, 6, 7, 8, 9, or 10.
   R* is succinimide
   L₁ is —[CH₂]₁₋₃—C(O)NH—.
   The drug moiety is selected from exatecan having the formula:

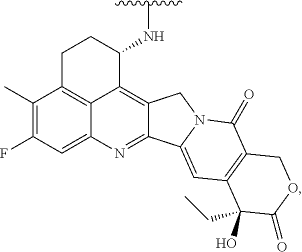 [SMILES: *N[C@H]1CCc2c(C)c(F)cc3nc4c(c1c23)Cn1c-4cc2c(c1=O)COC(=O)[C@]2(O)CC]

Dxd, Sn-38, monomethyl auristatin E (MMAE), and a pyrridinobenzodiazepine (PDD).

In one embodiment (XX), the disclosure provides an antibody-drug conjugate having formula (I):

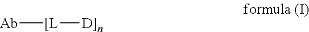

wherein in formula (I):
Ab is an antibody or antibody-binding fragment;
L is a linker;
D is a drug moiety; and
n is an integer from 1 to 20.
   Ab is an antibody or binding fragment thereof that specifically binds CUB Domain-Containing Protein-1 (CDCP1).
   The linker has the following formula:

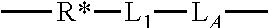

wherein L_{A} is a linker, L₁ is a linking moiety, and R* is a reactive moiety or a targeting agent.
The linker L_{A} has the formula:

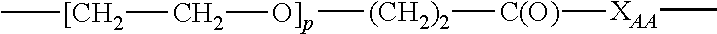

wherein
X_{AA} is a nonpolar dipeptide,
p is an integer from 5 to 10, e.g. 5, 6, 7, 8, 9, or 10.
   R* is succinimide
   L₁ is —[CH₂]₁₋₃—C(O)NH—.
   The drug moiety is selected from exatecan having the formula:

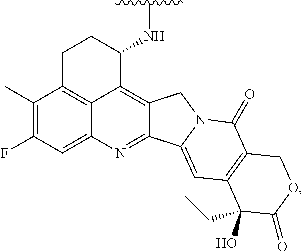 [SMILES: *N[C@H]1CCc2c(C)c(F)cc3nc4c(c1c23)Cn1c-4cc2c(c1=O)COC(=O)[C@]2(O)CC]

Dxd, Sn-38, monomethyl auristatin E (MMAE), and a pyrridinobenzodiazepine (PDD).

In one embodiment (XXI), the disclosure provides an antibody-drug conjugate having formula (I):

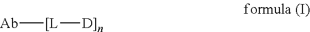

wherein in formula (I):
Ab is an antibody or antibody-binding fragment;
L is a linker;
D is a drug moiety; and
n is an integer from 1 to 20.
   The antibody or binding fragment thereof comprises
(i) a heavy chain variable region (VH) that comprises:
   (a) a VH complementarity determining region one (CDRH1) comprising and/or consisting of the amino acid sequence of SEQ ID NO: 2,
   (b) a VH complementarity determining region two (CDRH2) comprising and/or consisting of the amino acid sequence of SEQ ID NO: 3, and
   (c) a VH complementarity determining region three (CDRH3) comprising and/or consisting of the amino acid sequence of SEQ ID NO: 4,
and (ii) a light chain variable region (VL) that comprises:
   (a) a VL complementarity determining region one (CDRL1) comprising and/or consisting of the amino acid sequence of SEQ ID NO: 6,
   (b) a VL complementarity determining region two (CDRL2) comprising and/or consisting of the amino acid sequence of SEQ ID NO: 7, and
   (c) a VL complementarity determining region three (CDRL3) comprising and/or consisting of the amino acid sequence of SEQ ID NO:8.
      The linker has the following formula:

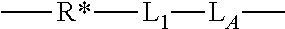

wherein L_{A} is a linker, L₁ is a linking moiety, and R* is a reactive moiety or a targeting agent.
The linker L_{A} has the formula:

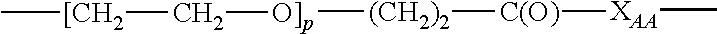

wherein
X_{AA} is a nonpolar dipeptide,
p is an integer from 5 to 10, e.g. 5, 6, 7, 8, 9, or 10.
   R* is succinimide
   L₁ is —[CH₂]₁₋₃—C(O)NH—.
   The drug moiety is selected from exatecan having the formula:

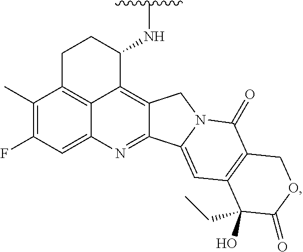 [SMILES: *N[C@H]1CCc2c(C)c(F)cc3nc4c(c1c23)Cn1c-4cc2c(c1=O)COC(=O)[C@]2(O)CC]

Dxd, Sn-38, monomethyl auristatin E (MMAE), and a pyrridinobenzodiazepine (PDD).

In one embodiment (XXII), the disclosure provides an antibody-drug conjugate having formula (I):

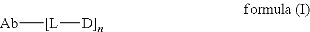

wherein in formula (I):
Ab is an antibody or antibody-binding fragment;
L is a linker;
D is a drug moiety; and
n is an integer from 1 to 20.
   The antibody or binding fragment thereof comprises
(i) a heavy chain variable region (VH) that comprises:
   (a) a VH complementarity determining region one (CDRH1) comprising and/or consisting of the amino acid sequence of SEQ ID NO: 12,
   (b) a VH complementarity determining region two (CDRH2) comprising and/or consisting of the amino acid sequence of SEQ ID NO: 13, and
   (c) a VH complementarity determining region three (CDRH3) comprising and/or consisting of the amino acid sequence of SEQ ID NO: 14,
and (ii) a light chain variable region (VL) that comprises:
   (a) a VL complementarity determining region one (CDRL1) comprising and/or consisting of the amino acid sequence of SEQ ID NO: 6,
   (b) a VL complementarity determining region two (CDRL2) comprising and/or consisting of the amino acid sequence of SEQ ID NO: 7, and
   (c) a VL complementarity determining region three (CDRL3) comprising and/or consisting of the amino acid sequence of SEQ ID NO:8.
      The linker has the following formula:

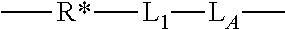

wherein L_{A} is a linker, L₁ is a linking moiety, and R* is a reactive moiety or a targeting agent.
The linker L_{A} has the formula:

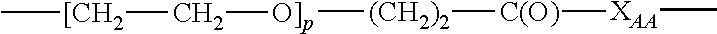

wherein
X_{AA} is a nonpolar dipeptide,
p is an integer from 5 to 10, e.g. 5, 6, 7, 8, 9, or 10.
   R* is succinimide.
   L₁ is —[CH₂]₁₋₃—C(O)NH—.
   The drug moiety is selected from exatecan having the formula:

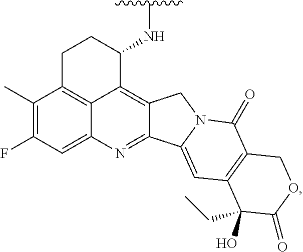 [SMILES: *N[C@H]1CCc2c(C)c(F)cc3nc4c(c1c23)Cn1c-4cc2c(c1=O)COC(=O)[C@]2(O)CC]

Dxd, Sn-38, monomethyl auristatin E (MMAE), and a pyrridinobenzodiazepine (PDD).

In one embodiment (XXIII), the disclosure provides an antibody-drug conjugate having formula (I):

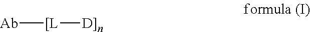

wherein in formula (I):
Ab is an antibody or antibody-binding fragment;
L is a linker;
D is a drug moiety; and
n is an integer from 1 to 20.
   The antibody or binding fragment thereof Ab comprises a VH that comprises and/or consists of an amino acid sequence of SEQ ID NO: 1 and/or comprises a VL that comprises and/or consists of an amino acid sequence of SEQ ID NO: 5.
   The linker has the following formula:

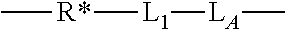

wherein L_{A} is a linker, L₁ is a linking moiety, and R* is a reactive moiety or a targeting agent.
The linker L_{A} has the formula:

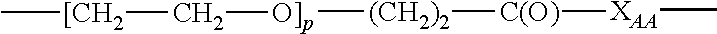

wherein
X_{AA} is a nonpolar dipeptide,
p is an integer from 5 to 10, e.g. 5, 6, 7, 8, 9, or 10.
   R* is succinimide.
   L₁ is —[CH₂]₁₋₃—C(O)NH—.
   The drug moiety is selected from exatecan having the formula:

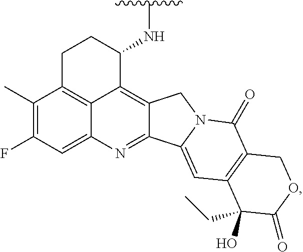 [SMILES: *N[C@H]1CCc2c(C)c(F)cc3nc4c(c1c23)Cn1c-4cc2c(c1=O)COC(=O)[C@]2(O)CC]

Dxd, Sn-38, monomethyl auristatin E (MMAE), and a pyrridinobenzodiazepine (PDD).

In one embodiment (XXIV), the disclosure provides an antibody-drug conjugate having formula (I):

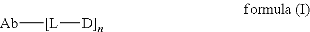

wherein in formula (I):
Ab is an antibody or antibody-binding fragment;
L is a linker;
D is a drug moiety; and
n is an integer from 1 to 20.
   The antibody or binding fragment thereof Ab comprises a heavy chain that comprises an amino acid sequence of SEQ ID NO: 1 and/or comprises a light chain that comprises an amino acid sequence of SEQ ID NO: 5.
   The linker has the following formula:

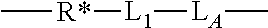

wherein L_{A} is a linker, L₁ is a linking moiety, and R* is a reactive moiety or a targeting agent.
The linker L_{A} has the formula:

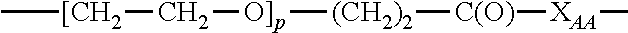

wherein
X_{AA} is a nonpolar dipeptide,
p is an integer from 5 to 10, e.g. 5, 6, 7, 8, 9, or 10.
   R* is succinimide.
   L₁ is —[CH₂]₁₋₃—C(O)NH—.
   The drug moiety is selected from exatecan having the formula:

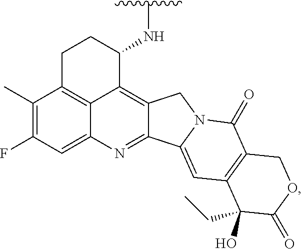 [SMILES: *N[C@H]1CCc2c(C)c(F)cc3nc4c(c1c23)Cn1c-4cc2c(c1=O)COC(=O)[C@]2(O)CC]

Dxd, Sn-38, monomethyl auristatin E (MMAE), and a pyrridinobenzodiazepine (PDD).

In one embodiment (XXV), the disclosure provides an antibody-drug conjugate having formula (I):

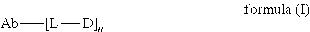

wherein in formula (I):
Ab is an antibody or antibody-binding fragment;
L is a linker;
D is a drug moiety; and
n is an integer from 1 to 10, e.g. 1, 2, 3, 4, 5, 6, 7, 8, 9, or 10.
   The linker has the following formula:

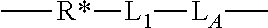

wherein L_{A} is a linker, L₁ is a linking moiety, and R* is a reactive moiety or a targeting agent.
The linker L_{A} has the formula:

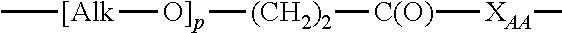

wherein
Alk designates C₂-C₄-alkylene,
X_{AA} is Val-Ala,
p is 7 or 8.
   R* is a reactive moiety selected from a succinimide, triazole, an amide, and a thioether.
   L₁ is —[CH₂]₀₋₁₂—C(O)NH—.
   The drug moiety is selected from exatecan having the formula:

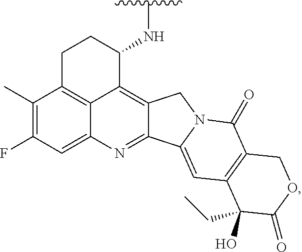 [SMILES: *N[C@H]1CCc2c(C)c(F)cc3nc4c(c1c23)Cn1c-4cc2c(c1=O)COC(=O)[C@]2(O)CC]

Dxd, Sn-38, monomethyl auristatin E (MMAE), and a pyrridinobenzodiazepine (PDD).

In one embodiment (XVI), the disclosure provides an antibody-drug conjugate having formula (I):

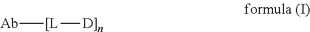

wherein in formula (I):
Ab is an antibody or antibody-binding fragment;
L is a linker;
D is a drug moiety; and
n is an integer from 1 to 10, e.g. 1, 2, 3, 4, 5, 6, 7, 8, 9, or 10.
   Ab is an antibody or binding fragment thereof that specifically binds CUB Domain-Containing Protein-1 (CDCP1).
   The linker has the following formula:

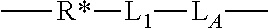

wherein L_{A} is a linker, L₁ is a linking moiety, and R* is a reactive moiety or a targeting agent.
The linker L_{A} has the formula:

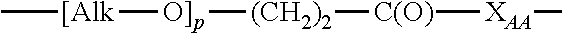

wherein
Alk designates C₂-C₄-alkylene,
X_{AA} is Val-Ala,
p is 7 or 8.
   R* is a reactive moiety selected from a succinimide, triazole, an amide, and a thioether.
   L₁ is —[CH₂]₀₋₁₂—C(O)NH—.
   The drug moiety is selected from exatecan having the formula:

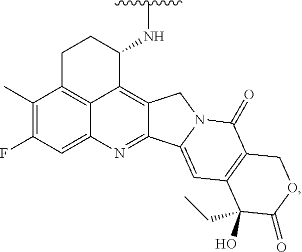 [SMILES: *N[C@H]1CCc2c(C)c(F)cc3nc4c(c1c23)Cn1c-4cc2c(c1=O)COC(=O)[C@]2(O)CC]

Dxd, Sn-38, monomethyl auristatin E (MMAE), and a pyrridinobenzodiazepine (PDD).

In one embodiment (XXVII), the disclosure provides an antibody-drug conjugate having formula (I):

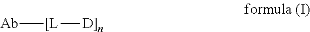

wherein in formula (I):
Ab is an antibody or antibody-binding fragment;
L is a linker;
D is a drug moiety; and
n is an integer from 1 to 10, e.g. 1, 2, 3, 4, 5, 6, 7, 8, 9, or 10.
   The antibody or binding fragment thereof comprises
(i) a heavy chain variable region (VH) that comprises:
   (a) a VH complementarity determining region one (CDRH1) comprising and/or consisting of the amino acid sequence of SEQ ID NO: 2,
   (b) a VH complementarity determining region two (CDRH2) comprising and/or consisting of the amino acid sequence of SEQ ID NO: 3, and
   (c) a VH complementarity determining region three (CDRH3) comprising and/or consisting of the amino acid sequence of SEQ ID NO: 4,
and (ii) a light chain variable region (VL) that comprises:
   (a) a VL complementarity determining region one (CDRL1) comprising and/or consisting of the amino acid sequence of SEQ ID NO: 6,
   (b) a VL complementarity determining region two (CDRL2) comprising and/or consisting of the amino acid sequence of SEQ ID NO: 7, and
   (c) a VL complementarity determining region three (CDRL3) comprising and/or consisting of the amino acid sequence of SEQ ID NO:8.
      The linker has the following formula:

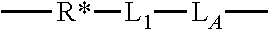

wherein L_{A} is a linker, L₁ is a linking moiety, and R* is a reactive moiety or a targeting agent.
The linker L_{A} has the formula:

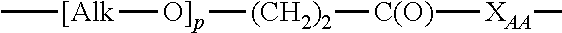

wherein
Alk designates C₂-C₄-alkylene,
X_{AA} is Val-Ala,
p is 7 or 8.
   R* is a reactive moiety selected from a succinimide, triazole, an amide, and a thioether.
   L₁ is —[CH₂]₀₋₁₂—C(O)NH—.
   The drug moiety is selected from exatecan having the formula:

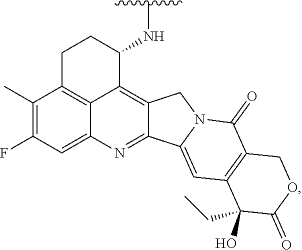 [SMILES: *N[C@H]1CCc2c(C)c(F)cc3nc4c(c1c23)Cn1c-4cc2c(c1=O)COC(=O)[C@]2(O)CC]

Dxd, Sn-38, monomethyl auristatin E (MMAE), and a pyrridinobenzodiazepine (PDD).

In one embodiment (XXVIII), the disclosure provides an antibody-drug conjugate having formula (I):

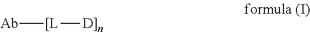

wherein in formula (I):
Ab is an antibody or antibody-binding fragment;
L is a linker;
D is a drug moiety; and
n is an integer from 1 to 10, e.g. 1, 2, 3, 4, 5, 6, 7, 8, 9, or 10.
   The antibody or binding fragment thereof comprises
(i) a heavy chain variable region (VH) that comprises:
   (a) a VH complementarity determining region one (CDRH1) comprising and/or consisting of the amino acid sequence of SEQ ID NO: 12,
   (b) a VH complementarity determining region two (CDRH2) comprising and/or consisting of the amino acid sequence of SEQ ID NO: 13, and
   (c) a VH complementarity determining region three (CDRH3) comprising and/or consisting of the amino acid sequence of SEQ ID NO: 14,
and (ii) a light chain variable region (VL) that comprises:
   (a) a VL complementarity determining region one (CDRL1) comprising and/or consisting of the amino acid sequence of SEQ ID NO: 6,
   (b) a VL complementarity determining region two (CDRL2) comprising and/or consisting of the amino acid sequence of SEQ ID NO: 7, and
   (c) a VL complementarity determining region three (CDRL3) comprising and/or consisting of the amino acid sequence of SEQ ID NO:8.
      The linker has the following formula:

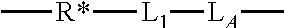

wherein L_{A} is a linker, L₁ is a linking moiety, and R* is a reactive moiety or a targeting agent.
The linker L_{A} has the formula:

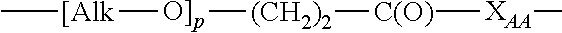

wherein
Alk designates C₂-C₄-alkylene,
X_{AA} is Val-Ala,
p is 7 or 8.
   R* is a reactive moiety selected from a succinimide, triazole, an amide, and a thioether.
   L₁ is —[CH₂]₀₋₁₂—C(O)NH—.
   The drug moiety is selected from exatecan having the formula:

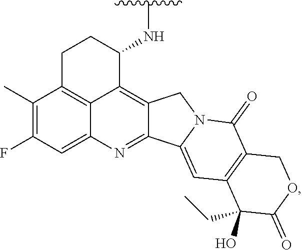 [SMILES: *N[C@H]1CCc2c(C)c(F)cc3nc4c(c1c23)Cn1c-4cc2c(c1=O)COC(=O)[C@]2(O)CC]

Dxd, Sn-38, monomethyl auristatin E (MMAE), and a pyrridinobenzodiazepine (PDD).

In one embodiment (XXIX), the disclosure provides an antibody-drug conjugate having formula (I):

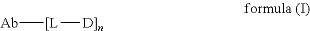

wherein in formula (I):
Ab is an antibody or antibody-binding fragment;
L is a linker;
D is a drug moiety; and
n is an integer from 1 to 10, e.g. 1, 2, 3, 4, 5, 6, 7, 8, 9, or 10.
   The antibody or binding fragment thereof Ab comprises a VH that comprises and/or consists of an amino acid sequence of SEQ ID NO: 1 and/or comprises a VL that comprises and/or consists of an amino acid sequence of SEQ ID NO: 5.
   The linker has the following formula:

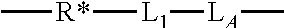

wherein L_{A} is a linker, L₁ is a linking moiety, and R* is a reactive moiety or a targeting agent.
The linker L_{A} has the formula:

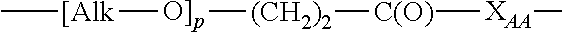

wherein
Alk designates C₂-C₄-alkylene,
X_{AA} is Val-Ala,
p is 7 or 8.
   R* is a reactive moiety selected from a succinimide, triazole, an amide, and a thioether.
   L₁ is —[CH₂]₀₋₁₂—C(O)NH—.
   The drug moiety is selected from exatecan having the formula:

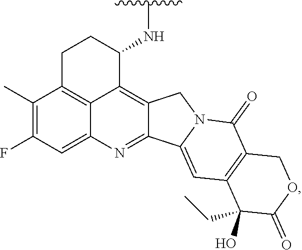 [SMILES: *N[C@H]1CCc2c(C)c(F)cc3nc4c(c1c23)Cn1c-4cc2c(c1=O)COC(=O)[C@]2(O)CC]

Dxd, Sn-38, monomethyl auristatin E (MMAE), and a pyrridinobenzodiazepine (PDD).

In one embodiment (XXX), the disclosure provides an antibody-drug conjugate having formula (I):

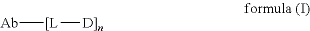

wherein in formula (I):
Ab is an antibody or antibody-binding fragment;
L is a linker;
D is a drug moiety; and
n is an integer from 1 to 10, e.g. 1, 2, 3, 4, 5, 6, 7, 8, 9, or 10.
   The antibody or binding fragment thereof Ab comprises a heavy chain that comprises an amino acid sequence of SEQ ID NO: 1 and/or comprises a light chain that comprises an amino acid sequence of SEQ ID NO: 5.
   The linker has the following formula:

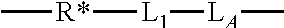

wherein L_{A} is a linker, L₁ is a linking moiety, and R* is a reactive moiety or a targeting agent.
The linker L_{A} has the formula:

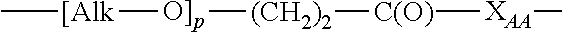

wherein
Alk designates C₂-C₄-alkylene,
X_{AA} is Val-Ala,
p is 7 or 8.
   R* is a reactive moiety selected from a succinimide, triazole, an amide, and a thioether.
   L₁ is —[CH₂]₀₋₁₂—C(O)NH—.
   The drug moiety is selected from exatecan having the formula:

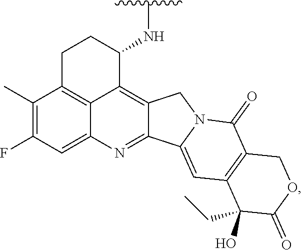 [SMILES: *N[C@H]1CCc2c(C)c(F)cc3nc4c(c1c23)Cn1c-4cc2c(c1=O)COC(=O)[C@]2(O)CC]

Dxd, Sn-38, monomethyl auristatin E (MMAE), and a pyrridinobenzodiazepine (PDD).

In one embodiment (XXXI), the disclosure provides an antibody-drug conjugate having formula (I):

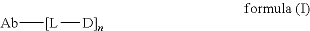

wherein in formula (I):
Ab is an antibody or antibody-binding fragment;
L is a linker;
D is a drug moiety; and
n is an integer from 1 to 10, e.g. 1, 2, 3, 4, 5, 6, 7, 8, 9, or 10.
   The linker has the following formula:

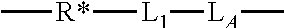

wherein L_{A} is a linker, L₁ is a linking moiety, and R* is a reactive moiety or a targeting agent.
The linker L_{A} has the formula:

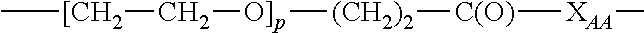

wherein
X_{AA} is Val-Ala,
p is 7 or 8.
   R* is a reactive moiety selected from a succinimide, triazole, an amide, and a thioether.
   L₁ is —[CH₂]₀₋₁₂—C(O)NH—.
   The drug moiety is selected from exatecan having the formula:

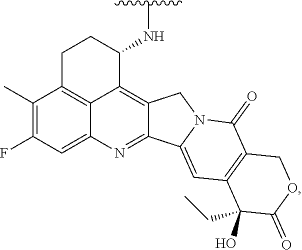 [SMILES: *N[C@H]1CCc2c(C)c(F)cc3nc4c(c1c23)Cn1c-4cc2c(c1=O)COC(=O)[C@]2(O)CC]

Dxd, Sn-38, monomethyl auristatin E (MMAE), and a pyrridinobenzodiazepine (PDD).

In one embodiment (XXXII), the disclosure provides an antibody-drug conjugate having formula (I):

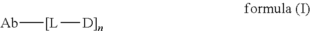

wherein in formula (I):
Ab is an antibody or antibody-binding fragment;
L is a linker;
D is a drug moiety; and
n is an integer from 1 to 10, e.g. 1, 2, 3, 4, 5, 6, 7, 8, 9, or 10.
   Ab is an antibody or binding fragment thereof that specifically binds CUB Domain-Containing Protein-1 (CDCP1).
   The linker has the following formula:

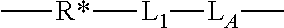

wherein L_{A} is a linker, L₁ is a linking moiety, and R* is a reactive moiety or a targeting agent.
The linker L_{A} has the formula:

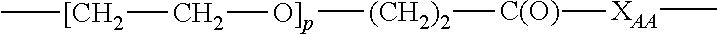

wherein
X_{AA} is Val-Ala,
p is 7 or 8.
   R* is a reactive moiety selected from a succinimide, triazole, an amide, and a thioether.
   L₁ is —[CH₂]₀₋₁₂—C(O)NH—.
   The drug moiety is selected from exatecan having the formula:

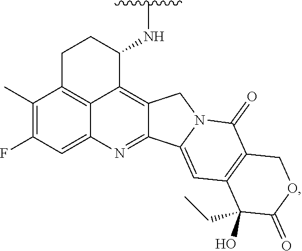 [SMILES: *N[C@H]1CCc2c(C)c(F)cc3nc4c(c1c23)Cn1c-4cc2c(c1=O)COC(=O)[C@]2(O)CC]

Dxd, Sn-38, monomethyl auristatin E (MMAE), and a pyrridinobenzodiazepine (PDD).

In one embodiment (XXXIII), the disclosure provides an antibody-drug conjugate having formula (I):

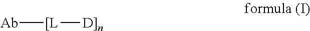

wherein in formula (I):
Ab is an antibody or antibody-binding fragment;
L is a linker;
D is a drug moiety; and
n is an integer from 1 to 10, e.g. 1, 2, 3, 4, 5, 6, 7, 8, 9, or 10.
   The antibody or binding fragment thereof comprises
(i) a heavy chain variable region (VH) that comprises:
   (a) a VH complementarity determining region one (CDRH1) comprising and/or consisting of the amino acid sequence of SEQ ID NO: 2,
   (b) a VH complementarity determining region two (CDRH2) comprising and/or consisting of the amino acid sequence of SEQ ID NO: 3, and
   (c) a VH complementarity determining region three (CDRH3) comprising and/or consisting of the amino acid sequence of SEQ ID NO: 4,
and (ii) a light chain variable region (VL) that comprises:
   (a) a VL complementarity determining region one (CDRL1) comprising and/or consisting of the amino acid sequence of SEQ ID NO: 6,
   (b) a VL complementarity determining region two (CDRL2) comprising and/or consisting of the amino acid sequence of SEQ ID NO: 7, and
   (c) a VL complementarity determining region three (CDRL3) comprising and/or consisting of the amino acid sequence of SEQ ID NO:8.
      The linker has the following formula:

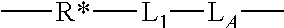

wherein L_{A} is a linker, L₁ is a linking moiety, and R* is a reactive moiety or a targeting agent.
The linker L_{A} has the formula:

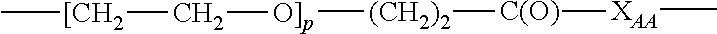

wherein
X_{AA} is Val-Ala,
p is 7 or 8.
   R* is a reactive moiety selected from a succinimide, triazole, an amide, and a thioether.
   L₁ is —[CH₂]₀₋₁₂—C(O)NH—.
   The drug moiety is selected from exatecan having the formula:

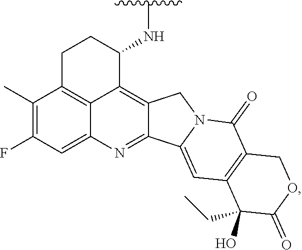 [SMILES: *N[C@H]1CCc2c(C)c(F)cc3nc4c(c1c23)Cn1c-4cc2c(c1=O)COC(=O)[C@]2(O)CC]

Dxd, Sn-38, monomethyl auristatin E (MMAE), and a pyrridinobenzodiazepine (PDD).

In one embodiment (XXXIV), the disclosure provides an antibody-drug conjugate having formula (I):

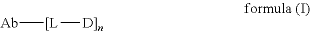

wherein in formula (I):
Ab is an antibody or antibody-binding fragment;
L is a linker;
D is a drug moiety; and
n is an integer from 1 to 10, e.g. 1, 2, 3, 4, 5, 6, 7, 8, 9, or 10.
   The antibody or binding fragment thereof comprises
(i) a heavy chain variable region (VH) that comprises:
   (a) a VH complementarity determining region one (CDRH1) comprising and/or consisting of the amino acid sequence of SEQ ID NO: 12,
   (b) a VH complementarity determining region two (CDRH2) comprising and/or consisting of the amino acid sequence of SEQ ID NO: 13, and
   (c) a VH complementarity determining region three (CDRH3) comprising and/or consisting of the amino acid sequence of SEQ ID NO: 14,
and (ii) a light chain variable region (VL) that comprises:
   (a) a VL complementarity determining region one (CDRL1) comprising and/or consisting of the amino acid sequence of SEQ ID NO: 6,
   (b) a VL complementarity determining region two (CDRL2) comprising and/or consisting of the amino acid sequence of SEQ ID NO: 7, and
   (c) a VL complementarity determining region three (CDRL3) comprising and/or consisting of the amino acid sequence of SEQ ID NO:8.
      The linker has the following formula:

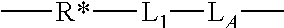

wherein L_{A} is a linker, L₁ is a linking moiety, and R* is a reactive moiety or a targeting agent.
The linker L_{A} has the formula:

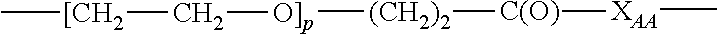

wherein
X_{AA} is Val-Ala,
p is 7 or 8.
   R* is a reactive moiety selected from a succinimide, triazole, an amide, and a thioether.
   L₁ is —[CH₂]₀₋₁₂—C(O)NH—.
   The drug moiety is selected from exatecan having the formula:

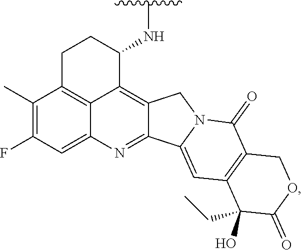 [SMILES: *N[C@H]1CCc2c(C)c(F)cc3nc4c(c1c23)Cn1c-4cc2c(c1=O)COC(=O)[C@]2(O)CC]

Dxd, Sn-38, monomethyl auristatin E (MMAE), and a pyrridinobenzodiazepine (PDD).

In one embodiment (XXXV), the disclosure provides an antibody-drug conjugate having formula (I):

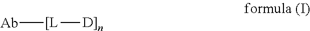

wherein in formula (I):
Ab is an antibody or antibody-binding fragment;
L is a linker;
D is a drug moiety; and
n is an integer from 1 to 10, e.g. 1, 2, 3, 4, 5, 6, 7, 8, 9, or 10.
   The antibody or binding fragment thereof Ab comprises a VH that comprises and/or consists of an amino acid sequence of SEQ ID NO: 1 and/or comprises a VL that comprises and/or consists of an amino acid sequence of SEQ ID NO: 5.
   The linker has the following formula:

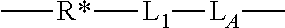

wherein L_{A} is a linker, L₁ is a linking moiety, and R* is a reactive moiety or a targeting agent.
The linker L_{A} has the formula:

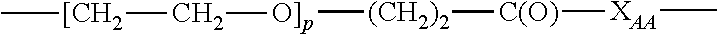

wherein
X_{AA} is Val-Ala,
p is 7 or 8.
   R* is a reactive moiety selected from a succinimide, triazole, an amide, and a thioether.
   L₁ is —[CH₂]₀₋₁₂—C(O)NH—.
   The drug moiety is selected from exatecan having the formula:

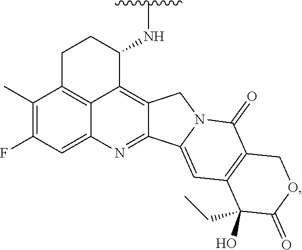 [SMILES: *N[C@H]1CCc2c(C)c(F)cc3nc4c(c1c23)Cn1c-4cc2c(c1=O)COC(=O)[C@]2(O)CC]

Dxd, Sn-38, monomethyl auristatin E (MMAE), and a pyrridinobenzodiazepine (PDD).

In one embodiment (XXXVI), the disclosure provides an antibody-drug conjugate having formula (I):

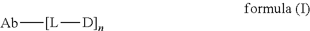

wherein in formula (I):
Ab is an antibody or antibody-binding fragment;
L is a linker;
D is a drug moiety; and
n is an integer from 1 to 10, e.g. 1, 2, 3, 4, 5, 6, 7, 8, 9, or 10.
   The antibody or binding fragment thereof Ab comprises a heavy chain that comprises an amino acid sequence of SEQ ID NO: 1 and/or comprises a light chain that comprises an amino acid sequence of SEQ ID NO: 5.
   The linker has the following formula:

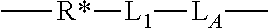

wherein L_{A} is a linker, L₁ is a linking moiety, and R* is a reactive moiety or a targeting agent.
The linker L_{A} has the formula:

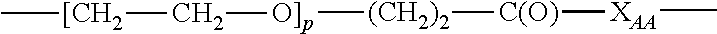

wherein
X_{AA} is Val-Ala,
p is 7 or 8.
   R* is a reactive moiety selected from a succinimide, triazole, an amide, and a thioether.
   L₁ is —[CH₂]₀₋₁₂—C(O)NH—.
   The drug moiety is selected from exatecan having the formula:

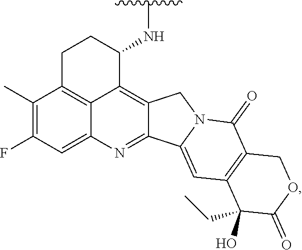 [SMILES: *N[C@H]1CCc2c(C)c(F)cc3nc4c(c1c23)Cn1c-4cc2c(c1=O)COC(=O)[C@]2(O)CC]

Dxd, Sn-38, monomethyl auristatin E (MMAE), and a pyrridinobenzodiazepine (PDD).

In one embodiment (XXXVII), the disclosure provides an antibody-drug conjugate having formula (I):

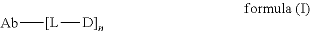

wherein in formula (I):
Ab is an antibody or antibody-binding fragment;
L is a linker;
D is a drug moiety; and
n is an integer from 1 to 10, e.g. 1, 2, 3, 4, 5, 6, 7, 8, 9, or 10.
   The linker has the following formula:

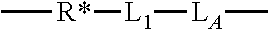

wherein L_{A} is a linker, L₁ is a linking moiety, and R* is a reactive moiety or a targeting agent.
The linker L_{A} has the formula:

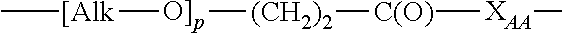

wherein
Alk designates C₂-C₄-alkylene,
X_{AA} is Val-Ala,
p is 7 or 8.
   R* is succinimide.
   L₁ is —[CH₂]₁₋₃—C(O)NH—.
   The drug moiety is selected from exatecan having the formula:

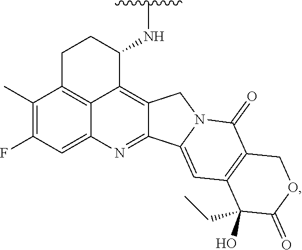 [SMILES: *N[C@H]1CCc2c(C)c(F)cc3nc4c(c1c23)Cn1c-4cc2c(c1=O)COC(=O)[C@]2(O)CC]

Dxd, Sn-38, monomethyl auristatin E (MMAE), and a pyrridinobenzodiazepine (PDD).

In one embodiment (XXXVIII), the disclosure provides an antibody-drug conjugate having formula (I):

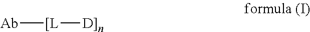

wherein in formula (I):
Ab is an antibody or antibody-binding fragment;
L is a linker;
D is a drug moiety; and
n is an integer from 1 to 10, e.g. 1, 2, 3, 4, 5, 6, 7, 8, 9, or 10.
   Ab is an antibody or binding fragment thereof that specifically binds CUB Domain-Containing Protein-1 (CDCP1).
   The linker has the following formula:

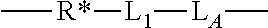

wherein L_{A} is a linker, L₁ is a linking moiety, and R* is a reactive moiety or a targeting agent.

The linker L_{A} has the formula:

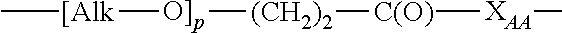

wherein
Alk designates C₂-C₄-alkylene,
X_{AA} is Val-Ala,
p is 7 or 8.
   R* is succinimide.
   L₁ is —[CH₂]₁₋₃—C(O)NH—.
   The drug moiety is selected from exatecan having the formula:

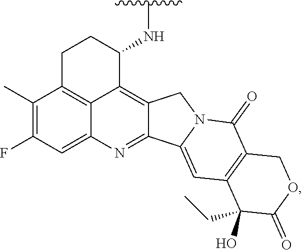 [SMILES: *N[C@H]1CCc2c(C)c(F)cc3nc4c(c1c23)Cn1c-4cc2c(c1=O)COC(=O)[C@]2(O)CC]

Dxd, Sn-38, monomethyl auristatin E (MMAE), and a pyrridinobenzodiazepine (PDD).

In one embodiment (XXXIX), the disclosure provides an antibody-drug conjugate having formula (I):

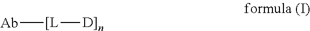

wherein in formula (I):
Ab is an antibody or antibody-binding fragment;
L is a linker;
D is a drug moiety; and
n is an integer from 1 to 10, e.g. 1, 2, 3, 4, 5, 6, 7, 8, 9, or 10.
   The antibody or binding fragment thereof comprises
(i) a heavy chain variable region (VH) that comprises:
   (a) a VH complementarity determining region one (CDRH1) comprising and/or consisting of the amino acid sequence of SEQ ID NO: 2,
   (b) a VH complementarity determining region two (CDRH2) comprising and/or consisting of the amino acid sequence of SEQ ID NO: 3, and
   (c) a VH complementarity determining region three (CDRH3) comprising and/or consisting of the amino acid sequence of SEQ ID NO: 4,
and (ii) a light chain variable region (VL) that comprises:
   (a) a VL complementarity determining region one (CDRL1) comprising and/or consisting of the amino acid sequence of SEQ ID NO: 6,
   (b) a VL complementarity determining region two (CDRL2) comprising and/or consisting of the amino acid sequence of SEQ ID NO: 7, and
   (c) a VL complementarity determining region three (CDRL3) comprising and/or consisting of the amino acid sequence of SEQ ID NO:8.
      The linker has the following formula:

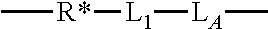

wherein L_{A} is a linker, L₁ is a linking moiety, and R* is a reactive moiety or a targeting agent.
The linker L_{A} has the formula:

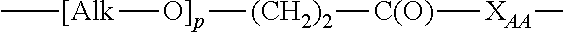

wherein
Alk designates C₂-C₄-alkylene,
X_{AA} is Val-Ala,
p is 7 or 8.
   R* is succinimide.
   L₁ is —[CH₂]₁₋₃—C(O)NH—.
   The drug moiety is selected from exatecan having the formula:

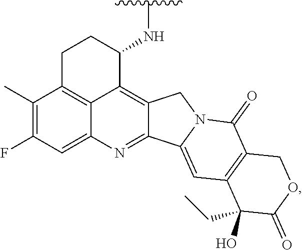 [SMILES: *N[C@H]1CCc2c(C)c(F)cc3nc4c(c1c23)Cn1c-4cc2c(c1=O)COC(=O)[C@]2(O)CC]

Dxd, Sn-38, monomethyl auristatin E (MMAE), and a pyrridinobenzodiazepine (PDD).

In one embodiment (XL), the disclosure provides an antibody-drug conjugate having formula (I):

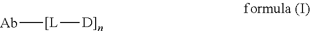

wherein in formula (I):
Ab is an antibody or antibody-binding fragment;
L is a linker;
D is a drug moiety; and
n is an integer from 1 to 10, e.g. 1, 2, 3, 4, 5, 6, 7, 8, 9, or 10.
   The antibody or binding fragment thereof comprises
(i) a heavy chain variable region (VH) that comprises:
   (a) a VH complementarity determining region one (CDRH1) comprising and/or consisting of the amino acid sequence of SEQ ID NO: 12,
   (b) a VH complementarity determining region two (CDRH2) comprising and/or consisting of the amino acid sequence of SEQ ID NO: 13, and
   (c) a VH complementarity determining region three (CDRH3) comprising and/or consisting of the amino acid sequence of SEQ ID NO: 14,
and (ii) a light chain variable region (VL) that comprises:
   (a) a VL complementarity determining region one (CDRL1) comprising and/or consisting of the amino acid sequence of SEQ ID NO: 6,
   (b) a VL complementarity determining region two (CDRL2) comprising and/or consisting of the amino acid sequence of SEQ ID NO: 7, and
   (c) a VL complementarity determining region three (CDRL3) comprising and/or consisting of the amino acid sequence of SEQ ID NO:8.
      The linker has the following formula:

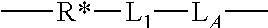

wherein L_{A} is a linker, L₁ is a linking moiety, and R* is a reactive moiety or a targeting agent.
The linker L_{A} has the formula:

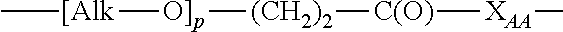

wherein
Alk designates C₂-C₄-alkylene,
X_{AA} is Val-Ala,
p is 7 or 8.
   R* is succinimide.
   L₁ is —[CH₂]₁₋₃—C(O)NH—.
   The drug moiety is selected from exatecan having the formula:

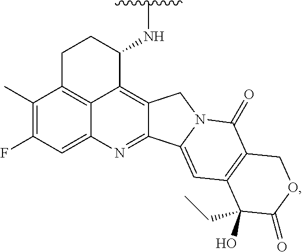 [SMILES: *N[C@H]1CCc2c(C)c(F)cc3nc4c(c1c23)Cn1c-4cc2c(c1=O)COC(=O)[C@]2(O)CC]

Dxd, Sn-38, monomethyl auristatin E (MMAE), and a pyrridinobenzodiazepine (PDD).

In one embodiment (XLI), the disclosure provides an antibody-drug conjugate having formula (I):

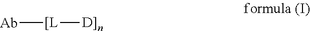

wherein in formula (I):
Ab is an antibody or antibody-binding fragment;
L is a linker;
D is a drug moiety; and
n is an integer from 1 to 10, e.g. 1, 2, 3, 4, 5, 6, 7, 8, 9, or 10.
   The antibody or binding fragment thereof Ab comprises a VH that comprises and/or consists of an amino acid sequence of SEQ ID NO: 1 and/or comprises a VL that comprises and/or consists of an amino acid sequence of SEQ ID NO: 5.
   The linker has the following formula:

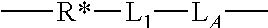

wherein L_{A} is a linker, L₁ is a linking moiety, and R* is a reactive moiety or a targeting agent.
The linker L_{A} has the formula:

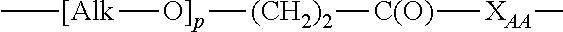

wherein
Alk designates C₂-C₄-alkylene,
X_{AA} is Val-Ala,
p is 7 or 8.
   R* is succinimide.
   L₁ is —[CH₂]₁₋₃—C(O)NH—.
   The drug moiety is selected from exatecan having the formula:

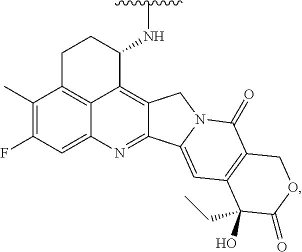 [SMILES: *N[C@H]1CCc2c(C)c(F)cc3nc4c(c1c23)Cn1c-4cc2c(c1=O)COC(=O)[C@]2(O)CC]

Dxd, Sn-38, monomethyl auristatin E (MMAE), and a pyrridinobenzodiazepine (PDD).

In one embodiment (XLII), the disclosure provides an antibody-drug conjugate having formula (I):

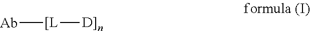

wherein in formula (I):
Ab is an antibody or antibody-binding fragment;
L is a linker;
D is a drug moiety; and
n is an integer from 1 to 10, e.g. 1, 2, 3, 4, 5, 6, 7, 8, 9, or 10.
   The antibody or binding fragment thereof Ab comprises a heavy chain that comprises an amino acid sequence of SEQ ID NO: 1 and/or comprises a light chain that comprises an amino acid sequence of SEQ ID NO: 5.
   The linker has the following formula:

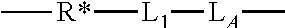

wherein L_{A} is a linker, L₁ is a linking moiety, and R* is a reactive moiety or a targeting agent.
The linker L_{A} has the formula:

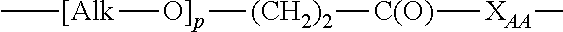

wherein
Alk designates C₂-C₄-alkylene,
X_{AA} is Val-Ala,
p is 7 or 8.
   R* is succinimide.
   L₁ is —[CH₂]₁₋₃—C(O)NH—.
   The drug moiety is selected from exatecan having the formula:

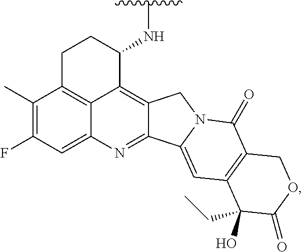 [SMILES: *N[C@H]1CCc2c(C)c(F)cc3nc4c(c1c23)Cn1c-4cc2c(c1=O)COC(=O)[C@]2(O)CC]

Dxd, Sn-38, monomethyl auristatin E (MMAE), and a pyrridinobenzodiazepine (PDD).

In one embodiment (XLIII), the disclosure provides an antibody-drug conjugate having formula (I):

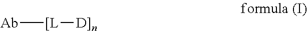

wherein in formula (I):
Ab is an antibody or antibody-binding fragment;
L is a linker;
D is a drug moiety; and
n is an integer from 1 to 10, e.g. 1, 2, 3, 4, 5, 6, 7, 8, 9, or 10.
   The linker has the following formula:

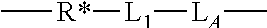

wherein L_{A} is a linker, L₁ is a linking moiety, and R* is a reactive moiety or a targeting agent.
The linker L_{A} has the formula:

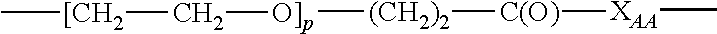

wherein
X_{AA} is Val-Ala,
p is 7 or 8.
   R* is succinimide.
   L₁ is —[CH₂]₁₋₃—C(O)NH—.
   The drug moiety is selected from exatecan having the formula:

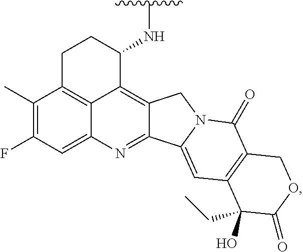 [SMILES: *N[C@H]1CCc2c(C)c(F)cc3nc4c(c1c23)Cn1c-4cc2c(c1=O)COC(=O)[C@]2(O)CC]

Dxd, Sn-38, monomethyl auristatin E (MMAE), and a pyrridinobenzodiazepine (PDD).

In one embodiment (XLIV), the disclosure provides an antibody-drug conjugate having formula (I):

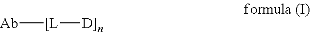

wherein in formula (I):
Ab is an antibody or antibody-binding fragment;
   L is a linker;
D is a drug moiety; and
n is an integer from 1 to 10, e.g. 1, 2, 3, 4, 5, 6, 7, 8, 9, or 10.
   Ab is an antibody or binding fragment thereof that specifically binds CUB Domain-Containing Protein-1 (CDCP1).
   The linker has the following formula:

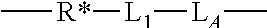

wherein L_{A} is a linker, L₁ is a linking moiety, and R* is a reactive moiety or a targeting agent.
The linker L_{A} has the formula:

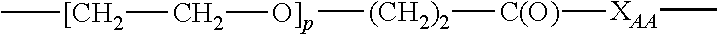

wherein
X_{AA} is Val-Ala,
p is 7 or 8.
   R* is succinimide.
   L₁ is —[CH₂]₁₋₃—C(O)NH—.
   The drug moiety is selected from exatecan having the formula:

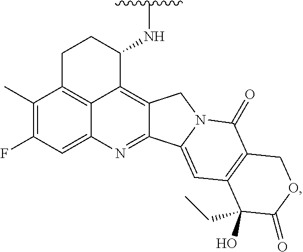 [SMILES: *N[C@H]1CCc2c(C)c(F)cc3nc4c(c1c23)Cn1c-4cc2c(c1=O)COC(=O)[C@]2(O)CC]

Dxd, Sn-38, monomethyl auristatin E (MMAE), and a pyrridinobenzodiazepine (PDD).

In one embodiment (XLV), the disclosure provides an antibody-drug conjugate having formula (I):

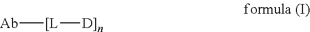

wherein in formula (I):
Ab is an antibody or antibody-binding fragment;
L is a linker;
D is a drug moiety; and
n is an integer from 1 to 10, e.g. 1, 2, 3, 4, 5, 6, 7, 8, 9, or 10.
   The antibody or binding fragment thereof comprises
(i) a heavy chain variable region (VH) that comprises:
   (a) a VH complementarity determining region one (CDRH1) comprising and/or consisting of the amino acid sequence of SEQ ID NO: 2,
   (b) a VH complementarity determining region two (CDRH2) comprising and/or consisting of the amino acid sequence of SEQ ID NO: 3, and
   (c) a VH complementarity determining region three (CDRH3) comprising and/or consisting of the amino acid sequence of SEQ ID NO: 4,
and (ii) a light chain variable region (VL) that comprises:
   (a) a VL complementarity determining region one (CDRL1) comprising and/or consisting of the amino acid sequence of SEQ ID NO: 6,
   (b) a VL complementarity determining region two (CDRL2) comprising and/or consisting of the amino acid sequence of SEQ ID NO: 7, and
   (c) a VL complementarity determining region three (CDRL3) comprising and/or consisting of the amino acid sequence of SEQ ID NO:8.
      The linker has the following formula:

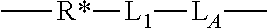

wherein L_{A} is a linker, L₁ is a linking moiety, and R* is a reactive moiety or a targeting agent.
The linker L_{A} has the formula:

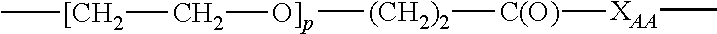

wherein
X_{AA} is Val-Ala,
p is 7 or 8.
   R* is succinimide.
   L₁ is —[CH₂]₁₋₃—C(O)NH—.
   The drug moiety is selected from exatecan having the formula:

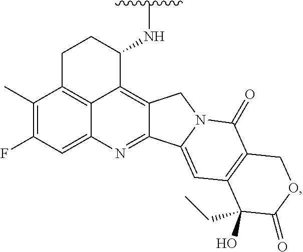 [SMILES: *N[C@H]1CCc2c(C)c(F)cc3nc4c(c1c23)Cn1c-4cc2c(c1=O)COC(=O)[C@]2(O)CC]

Dxd, Sn-38, monomethyl auristatin E (MMAE), and a pyrridinobenzodiazepine (PDD).

In one embodiment (XLVI), the disclosure provides an antibody-drug conjugate having formula (I):

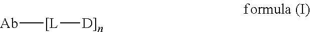

wherein in formula (I):
Ab is an antibody or antibody-binding fragment;
L is a linker;
D is a drug moiety; and
n is an integer from 1 to 10, e.g. 1, 2, 3, 4, 5, 6, 7, 8, 9, or 10.
   The antibody or binding fragment thereof comprises
(i) a heavy chain variable region (VH) that comprises:
   (a) a VH complementarity determining region one (CDRH1) comprising and/or consisting of the amino acid sequence of SEQ ID NO: 12,
   (b) a VH complementarity determining region two (CDRH2) comprising and/or consisting of the amino acid sequence of SEQ ID NO: 13, and
   (c) a VH complementarity determining region three (CDRH3) comprising and/or consisting of the amino acid sequence of SEQ ID NO: 14,
and (ii) a light chain variable region (VL) that comprises:
   (a) a VL complementarity determining region one (CDRL1) comprising and/or consisting of the amino acid sequence of SEQ ID NO: 6,
   (b) a VL complementarity determining region two (CDRL2) comprising and/or consisting of the amino acid sequence of SEQ ID NO: 7, and
   (c) a VL complementarity determining region three (CDRL3) comprising and/or consisting of the amino acid sequence of SEQ ID NO:8.
      The linker has the following formula:

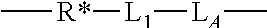

wherein L_{A} is a linker, L₁ is a linking moiety, and R* is a reactive moiety or a targeting agent.
The linker L_{A} has the formula:

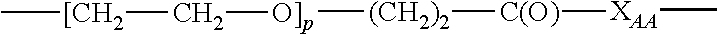

wherein
X_{AA} is Val-Ala,
p is 7 or 8.
   R* is succinimide.
   L₁ is —[CH₂]₁₋₃—C(O)NH—.
   The drug moiety is selected from exatecan having the formula:

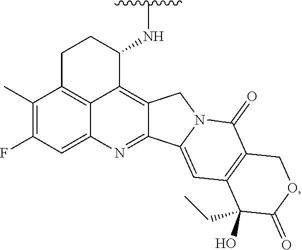 [SMILES: *N[C@H]1CCc2c(C)c(F)cc3nc4c(c1c23)Cn1c-4cc2c(c1=O)COC(=O)[C@]2(O)CC]

Dxd, Sn-38, monomethyl auristatin E (MMAE), and a pyrridinobenzodiazepine (PDD).

In one embodiment (XLVII), the disclosure provides an antibody-drug conjugate having formula (I):

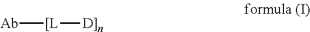

wherein in formula (I):
Ab is an antibody or antibody-binding fragment;
L is a linker;
D is a drug moiety; and
n is an integer from 1 to 10, e.g. 1, 2, 3, 4, 5, 6, 7, 8, 9, or 10.
   The antibody or binding fragment thereof Ab comprises a VH that comprises and/or consists of an amino acid sequence of SEQ ID NO: 1 and/or comprises a VL that comprises and/or consists of an amino acid sequence of SEQ ID NO: 5.
   The linker has the following formula:

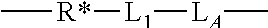

wherein L_{A} is a linker, L₁ is a linking moiety, and R* is a reactive moiety or a targeting agent.
The linker L_{A} has the formula:

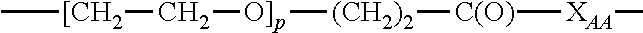

wherein
X_{AA} is Val-Ala,
p is 7 or 8.
   R* is succinimide.
   L₁ is —[CH₂]₁₋₃—C(O)NH—.
   The drug moiety is selected from exatecan having the formula:

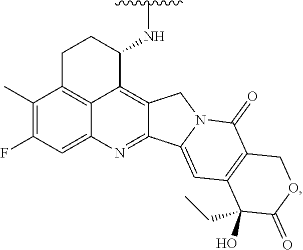 [SMILES: *N[C@H]1CCc2c(C)c(F)cc3nc4c(c1c23)Cn1c-4cc2c(c1=O)COC(=O)[C@]2(O)CC]

Dxd, Sn-38, monomethyl auristatin E (MMAE), and a pyrridinobenzodiazepine (PDD).

In one embodiment (XLVIII), the disclosure provides an antibody-drug conjugate having formula (I):

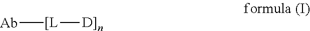

wherein in formula (I):
Ab is an antibody or antibody-binding fragment;
L is a linker;
D is a drug moiety; and
n is an integer from 1 to 10, e.g. 1, 2, 3, 4, 5, 6, 7, 8, 9, or 10.
   The antibody or binding fragment thereof Ab comprises a heavy chain that comprises an amino acid sequence of SEQ ID NO: 1 and/or comprises a light chain that comprises an amino acid sequence of SEQ ID NO: 5.
   The linker has the following formula:

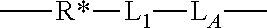

wherein L_{A} is a linker, L₁ is a linking moiety, and R* is a reactive moiety or a targeting agent.
The linker L_{A} has the formula:

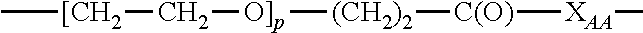

wherein
X_{AA} is Val-Ala,
p is 7 or 8.
   R* is succinimide.
   L₁ is —[CH₂]₁₋₃—C(O)NH—.
   The drug moiety is selected from exatecan having the formula:

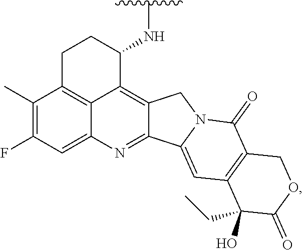 [SMILES: *N[C@H]1CCc2c(C)c(F)cc3nc4c(c1c23)Cn1c-4cc2c(c1=O)COC(=O)[C@]2(O)CC]

Dxd, Sn-38, monomethyl auristatin E (MMAE), and a pyrridinobenzodiazepine (PDD).

In one embodiment (XLIX), the disclosure provides an antibody-drug conjugate having formula (I):

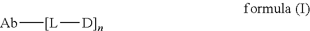

as defined in any one of embodiments (I) to (XLVIII), wherein index p is 8.

In one embodiment (L), the disclosure provides an antibody-drug conjugate having formula (I):

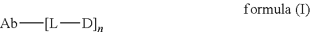

as defined in any of embodiments (I) to (XLIX), wherein L₁ is —[CH₂]₂—C(O)NH—.

In one embodiment (LI), the disclosure provides an antibody-drug conjugate having formula (I):

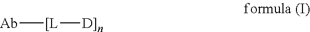

as defined in any one of embodiments (I) to (L), wherein the drug moiety is selected from exatecan having the formula:

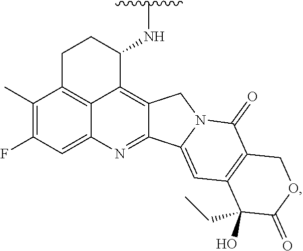 [SMILES: *N[C@H]1CCc2c(C)c(F)cc3nc4c(c1c23)Cn1c-4cc2c(c1=O)COC(=O)[C@]2(O)CC]

### Dxd, and SN-38.

In one embodiment (LII), the disclosure provides an antibody-drug conjugate having formula (I):

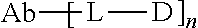

as defined in any one of embodiments (I) to (L), wherein the drug moiety is selected from exatecan having the formula:

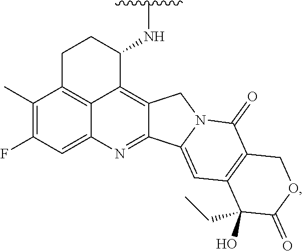 [SMILES: *N[C@H]1CCc2c(C)c(F)cc3nc4c(c1c23)Cn1c-4cc2c(c1=O)COC(=O)[C@]2(O)CC]

### Dxd, and SN-38.

In one embodiment (LII), the disclosure provides an antibody-drug conjugate having formula (I):

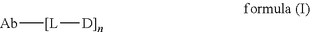

as defined in any one of embodiments (I) to (LI), wherein the drug moiety is exatecan having the formula:

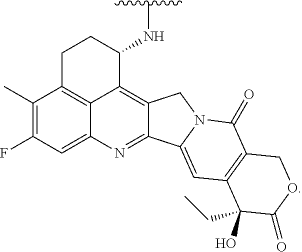 [SMILES: *N[C@H]1CCc2c(C)c(F)cc3nc4c(c1c23)Cn1c-4cc2c(c1=O)COC(=O)[C@]2(O)CC]

In one embodiment (LIII), the disclosure provides an antibody-drug conjugate having formula (I):

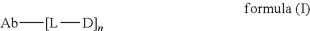

as defined in any one of embodiments (I) to (LII), wherein X_{AA} is not Val-Cit or Phe-Lys.

In one embodiment (LIV), the disclosure provides an antibody-drug conjugate having formula (I):

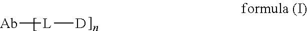

wherein the moiety -[L-D]ₙ has the following structure

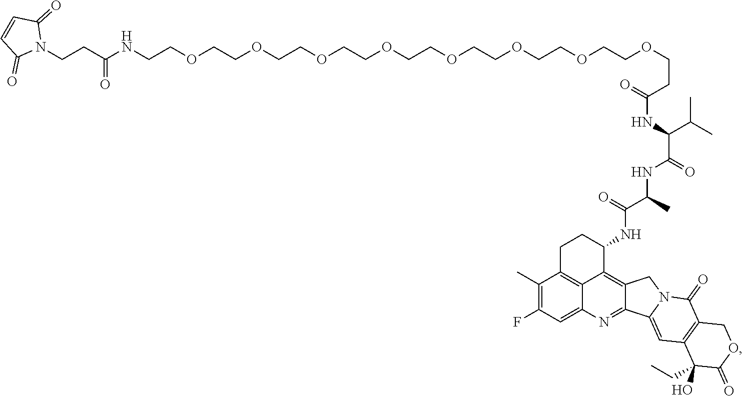 [SMILES: CC[C@@]1(O)C(=O)OCc2c1cc1n(c2=O)Cc2c-1nc1cc(F)c(C)c3c1c2[C@@H](NC(=O)[C@H](C)NC(=O)[C@@H](NC(=O)CCOCCOCCOCCOCCOCCOCCOCCOCCNC(=O)CCN1C(=O)C=CC1=O)C(C)C)CC3]

wherein the moiety Ab- is as defined in any one of embodiments (I) to (VI), and wherein said moiety Ab- is reacted with the maleimide group.

In one embodiment (LV), the disclosure provides a compound of formula (III), or salts, solvates, tautomers, isomers or mixtures thereof:

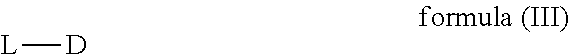

wherein in formula (III):
L is a linker; and
D comprises a drug moiety,
   said formula (III) corresponding to formula (I) in any one of embodiments (I) to (LIV),
   wherein Ab is absent and n is 1.

In embodiments, the disclosure provides an antibody-drug conjugate (ADC) having formula (I):

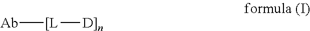

wherein in formula (I):
Ab is an antibody or antibody-binding fragment;
L is a linker having formula —R*-L₁-L_{A}-, wherein L_{A} is a linker, L₁ is a linking moiety, and R* is a reactive moiety or a targeting agent;
D is a drug moiety; and
n is an integer from 1 to 20. In embodiments, R* is a reactive moiety selected from a succinimide, triazole, an amide, and a thioether. In embodiments, R* is succinimide. In embodiments, L₁ is —[CH₂]₀₋₁₂—C(O)NH—. In some embodiments, the linker L_{A} has the formula -[Alk-O]ₚ—(CH₂)₂—C(O)—X_{AA}—. In some embodiments, the linker L_{A} has the formula —[CH₂CH₂—O]ₚ—(CH₂)₂—C(O)—X_{AA}—, wherein p is an integer from 5 to 10. In embodiments, X_{AA} is selected from Val-Ala, Tyr-Arg, Phe-Arg, Val-Gln, Val-Cit, Tyr-Met, Leu-Gln, Val-Arg, Met-Thr, Phe-Gln, Thr-Thr, Val-Thr, Ala-Ala, Val-Met, Leu-Met, Ala-Asn, D-Val-D-Gln, D-Ala-D-Ala, and Phe-Met. In some embodiments, X_{AA} is Val-Ala. In some embodiments, L₁ is —[CH₂]₁₋₃—C(O)NH—. In some embodiments, L₁ is —[CH₂]₂—C(O)NH—. In some embodiments, p is 7 or 8. In some embodiments, p is 8.

In some embodiments, the antibody or binding fragment thereof specifically binds CUB Domain-Containing Protein-1 (CDCP1).

In some embodiments, the antibody or binding fragment thereof Ab comprises:
(i) a heavy chain variable region (VH) that comprises one or more of:
   (a) a CDRH1 comprising and/or consisting of the amino acid sequence of SEQ ID NO: 2,
   (b) a CDRH2 comprising and/or consisting of the amino acid sequence of SEQ ID NO: 3, and
   (c) a CDRH3 comprising and/or consisting of the amino acid sequence of SEQ ID NO: 4,
and/or (ii) a light chain variable region (VL) that comprises one or more of:
   (a) a CDRL1 comprising and/or consisting of the amino acid sequence of SEQ ID NO: 6,
   (b) a CDRL2 comprising and/or consisting of the amino acid sequence of SEQ ID NO: 7, and
   (c) a CDRL3 comprising and/or consisting of the amino acid sequence of SEQ ID NO: 8.

In some embodiments, the antibody or binding fragment thereof Ab comprises a VH that comprises and/or consists of an amino acid sequence of SEQ ID NO: 1 and/or comprises a VL that comprises and/or consists of an amino acid sequence of SEQ ID NO: 5.

In some embodiments, the antibody or binding fragment thereof Ab comprises a heavy chain that comprises an amino acid sequence of SEQ ID NO: 1 and/or comprises a light chain that comprises an amino acid sequence of SEQ ID NO: 5.

In some embodiments, the antibody or binding fragment thereof Ab comprises:
(i) a heavy chain variable region (VH) that comprises one or more of:
   (a) a CDRH1 comprising and/or consisting of the amino acid sequence of SEQ ID NO: 2,
   (b) a CDRH2 comprising and/or consisting of the amino acid sequence of SEQ ID NO: 3, and
   (c) a CDRH3 comprising and/or consisting of the amino acid sequence of SEQ ID NO: 4,
and/or (ii) a light chain variable region (VL) that comprises one or more of:
   (a) a CDRL1 comprising and/or consisting of the amino acid sequence of SEQ ID NO: 6,
   (b) a CDRL2 comprising and/or consisting of the amino acid sequence of SEQ ID NO: 7, and
   (c) a CDRL3 comprising and/or consisting of the amino acid sequence of SEQ ID NO: 8, and the drug moiety D is or comprises exetecan.

In some embodiments, the antibody or binding fragment thereof Ab comprises:
(i) a heavy chain variable region (VH) that comprises one or more of:
   (a) a CDRH1 comprising and/or consisting of the amino acid sequence of SEQ ID NO: 2,
   (b) a CDRH2 comprising and/or consisting of the amino acid sequence of SEQ ID NO: 3, and
   (c) a CDRH3 comprising and/or consisting of the amino acid sequence of SEQ ID NO: 4,
and/or (ii) a light chain variable region (VL) that comprises one or more of:
   (a) a CDRL1 comprising and/or consisting of the amino acid sequence of SEQ ID NO: 6,
   (b) a CDRL2 comprising and/or consisting of the amino acid sequence of SEQ ID NO: 7, and
   (c) a CDRL3 comprising and/or consisting of the amino acid sequence of SEQ ID NO: 8, and wherein the drug moiety D is or comprises Dxd.

In some embodiments, the antibody or binding fragment thereof Ab comprises:
(i) a heavy chain variable region (VH) that comprises one or more of:
   (a) a CDRH1 comprising and/or consisting of the amino acid sequence of SEQ ID NO: 2,
   (b) a CDRH2 comprising and/or consisting of the amino acid sequence of SEQ ID NO: 3, and
   (c) a CDRH3 comprising and/or consisting of the amino acid sequence of SEQ ID NO: 4,
and/or (ii) a light chain variable region (VL) that comprises one or more of:
   (a) a CDRL1 comprising and/or consisting of the amino acid sequence of SEQ ID NO: 6,
   (b) a CDRL2 comprising and/or consisting of the amino acid sequence of SEQ ID NO: 7, and
   (c) a CDRL3 comprising and/or consisting of the amino acid sequence of SEQ ID NO: 8, and wherein the drug moiety D is or comprises MMAE. In some embodiments, the linker is or comprises val-cit. In some embodiments, the linker comprises val-cit-PAB.

In some embodiments, the antibody or binding fragment thereof Ab comprises:
(i) a heavy chain variable region (VH) that comprises one or more of:
   (a) a CDRH1 comprising and/or consisting of the amino acid sequence of SEQ ID NO: 2,
   (b) a CDRH2 comprising and/or consisting of the amino acid sequence of SEQ ID NO: 3, and
   (c) a CDRH3 comprising and/or consisting of the amino acid sequence of SEQ ID NO: 4,
and/or (ii) a light chain variable region (VL) that comprises one or more of:
   (a) a CDRL1 comprising and/or consisting of the amino acid sequence of SEQ ID NO: 6,
   (b) a CDRL2 comprising and/or consisting of the amino acid sequence of SEQ ID NO: 7, and
   (c) a CDRL3 comprising and/or consisting of the amino acid sequence of SEQ ID NO: 8, and wherein the drug moiety D is or comprises a pyrridinobenzodiazepine (PDD). In some embodiments, the PDD comprises or has the formula:

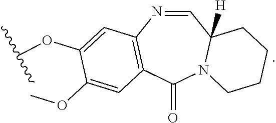 [SMILES: *Oc1cc2c(cc1OC)C(=O)N1CCCC[C@H]1C=N2]

In some embodiments, the PDD comprises or has the formula:

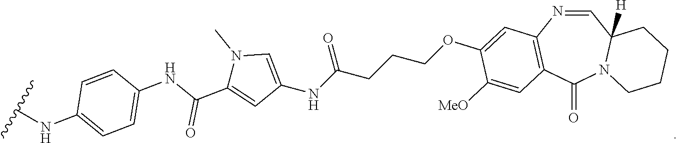 [SMILES: *Nc1ccc(NC(=O)c2cc(NC(=O)CCCOc3cc4c(cc3OC)C(=O)N3CCCC[C@H]3C=N4)cn2C)cc1]

In some embodiments, the PDD comprises or has the formula:

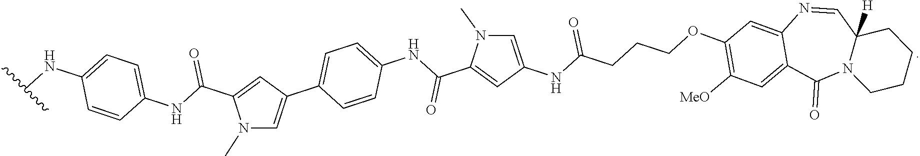 [SMILES: *Nc1ccc(NC(=O)c2cc(-c3ccc(NC(=O)c4cc(NC(=O)CCCOc5cc6c(cc5OC)C(=O)N5CCCC[C@H]5C=N6)cn4C)cc3)cn2C)cc1]

In some embodiments, the antibody or binding fragment thereof Ab comprises:
(i) a heavy chain variable region (VH) that comprises one or more of:
   (a) a CDRH1 comprising and/or consisting of the amino acid sequence of SEQ ID NO: 2,
   (b) a CDRH2 comprising and/or consisting of the amino acid sequence of SEQ ID NO: 3, and
   (c) a CDRH3 comprising and/or consisting of the amino acid sequence of SEQ ID NO: 4,
and/or (ii) a light chain variable region (VL) that comprises one or more of:
   (a) a CDRL1 comprising and/or consisting of the amino acid sequence of SEQ ID NO: 6,
   (b) a CDRL2 comprising and/or consisting of the amino acid sequence of SEQ ID NO: 7, and
   (c) a CDRL3 comprising and/or consisting of the amino acid sequence of SEQ ID NO: 8, and wherein the drug moiety D is or comprises Sn-38.

In some embodiments, the antibody or binding fragment thereof Ab comprises a VH that comprises and/or consists of the amino acid sequence of SEQ ID NO: 1 and a VL that comprises and/or consists of the amino acid sequence of SEQ ID NO: 5, and wherein the drug moiety D is or comprises exetecan.

In some embodiments, the antibody or binding fragment thereof Ab comprises a VH that comprises and/or consists of the amino acid sequence of SEQ ID NO: 1 and a VL that comprises and/or consists of the amino acid sequence of SEQ ID NO: 5, and wherein the drug moiety D is or comprises Dxd.

In some embodiments, the antibody or binding fragment thereof Ab comprises a VH that comprises and/or consists of the amino acid sequence of SEQ ID NO: 1 and a VL that comprises and/or consists of the amino acid sequence of SEQ ID NO: 5, and wherein the drug moiety D is or comprises MMAE. In some embodiments, the linker is or comprises val-cit. In some embodiments, the linker comprises val-cit-PAB.

In some embodiments, the antibody or binding fragment thereof Ab comprises a VH that comprises and/or consists of the amino acid sequence of SEQ ID NO: 1 and a VL that comprises and/or consists of the amino acid sequence of SEQ ID NO: 5, and wherein the drug moiety D is or comprises a pyrridinobenzodiazepine (PDD). In some embodiments, the PDD comprises or has the formula:

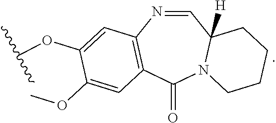 [SMILES: *Oc1cc2c(cc1OC)C(=O)N1CCCC[C@H]1C=N2]

In some embodiments, the PDD comprises or has the formula:

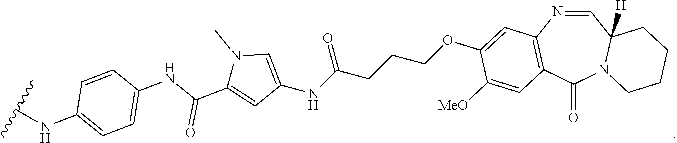 [SMILES: *Nc1ccc(NC(=O)c2cc(NC(=O)CCCOc3cc4c(cc3OC)C(=O)N3CCCC[C@H]3C=N4)cn2C)cc1]

In some embodiments, the PDD comprises or has the formula:

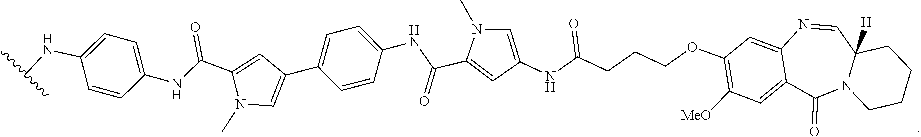 [SMILES: *Nc1ccc(NC(=O)c2cc(-c3ccc(NC(=O)c4cc(NC(=O)CCCOc5cc6c(cc5OC)C(=O)N5CCCC[C@H]5C=N6)cn4C)cc3)cn2C)cc1]

In some embodiments, the antibody or binding fragment thereof Ab comprises a VH that comprises and/or consists of the amino acid sequence of SEQ ID NO: 1 and a VL that comprises and/or consists of the amino acid sequence of SEQ ID NO: 5, and wherein the drug moiety D is or comprises Sn-38.

In some embodiments, the antibody or binding fragment thereof Ab comprises a heavy chain that comprises the amino acid sequence of SEQ ID NO: 1 and a light chain that comprises the amino acid sequence of SEQ ID NO: 5, and wherein the drug moiety D is or comprises exetecan.

In some embodiments, the antibody or binding fragment thereof Ab comprises a heavy chain that comprises the amino acid sequence of SEQ ID NO: 1 and a light chain that comprises the amino acid sequence of SEQ ID NO: 5, and wherein the drug moiety D is or comprises Dxd.

In some embodiments, the antibody or binding fragment thereof Ab comprises a heavy chain that comprises the amino acid sequence of SEQ ID NO: 1 and a light chain that comprises the amino acid sequence of SEQ ID NO: 5, and wherein the drug moiety D is or comprises MMAE.

In some embodiments, the antibody or binding fragment thereof Ab comprises a heavy chain that comprises the amino acid sequence of SEQ ID NO: 1 and a light chain that comprises the amino acid sequence of SEQ ID NO: 5, and wherein the drug moiety D is or comprises a pyrridinobenzodiazepine (PDD). In some embodiments, the PDD comprises or has the formula:

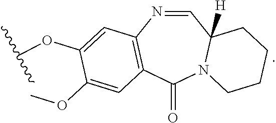 [SMILES: *Oc1cc2c(cc1OC)C(=O)N1CCCC[C@H]1C=N2]

In some embodiments, the PDD comprises or has the formula:

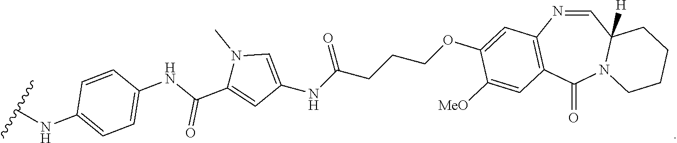 [SMILES: *Nc1ccc(NC(=O)c2cc(NC(=O)CCCOc3cc4c(cc3OC)C(=O)N3CCCC[C@H]3C=N4)cn2C)cc1]

In some embodiments, the PDD comprises or has the formula:

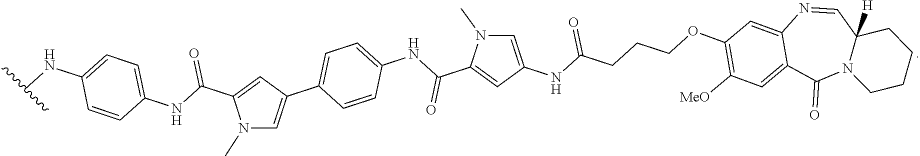 [SMILES: *Nc1ccc(NC(=O)c2cc(-c3ccc(NC(=O)c4cc(NC(=O)CCCOc5cc6c(cc5OC)C(=O)N5CCCC[C@H]5C=N6)cn4C)cc3)cn2C)cc1]

In some embodiments, the antibody or binding fragment thereof Ab comprises a heavy chain that comprises the amino acid sequence of SEQ ID NO: 1 and a light chain that comprises the amino acid sequence of SEQ ID NO: 5, and wherein the drug moiety D is or comprises Sn-38.

In some embodiments, the antibody is any antibody or binding fragment thereof disclosed herein. In some embodiments, the antibody or binding fragment thereof preferentially binds any portion of the polypeptide comprising SEQ ID NO: 9. In some embodiments, the antibody or binding fragment thereof is an anti-CDCP1 antibody.

In some embodiments, the anti-CDCP1 antibody comprises a CDR selected from the group consisting of SEQ ID NOs: 2, 3, 4, 6, 7, and 8.

In some embodiments, the conjugate is ADC1. In some embodiments, the conjugate is ADC2. In some embodiments, the conjugate is ADC3. In some embodiments, the conjugate is ADC4. In some embodiments, the conjugate is ADC5.

In some embodiments, the antibody drug conjugate as described herein binds CDCP1 at pH 7.4 with a K_{D} value of or less than about 1000 nM, about 500 nM, about 400 nM, about 300 nM, about 200 nM, about 180 nM, about 160 nM, about 140 nM, about 120 nM, or about 100 nM.

In some embodiments, ADCs may be produced or generated having (a) an antibody, or binding fragment thereof (e.g. an antibody, or binding fragment thereof that binds to CDCP1); (b) a linker and (c) a drug moiety. The drug-to-antibody ratio (DAR) or drug loading indicates the number of drug (D) molecules and/or moieties that are conjugated per antibody. In some embodiments, the number of linker-drug moieties attached to an antibody can be any number suitable for development of an ADC. In some embodiments, the number of linker-drug moieties per antibody ranges from about 1 to about 10. In some embodiments, the number of linker-drug moieties per antibody is about 10. In some embodiments, the number of linker-drug moieties per antibody is about 9. In some embodiments, the number of linker-drug moieties per antibody is about 8. In some embodiments, the number of linker-drug moieties per antibody is about 7. In some embodiments, the number of linker-drug moieties per antibody is about 6. In some embodiments, the number of linker-drug moieties per antibody is about 5. In some embodiments, the number of linker-drug moieties per antibody is about 4. In some embodiments, the number of linker-drug moieties per antibody is about 3. In some embodiments, the number of linker-drug moieties per antibody is about 2. In some embodiments, the number of linker-drug moieties per antibody is about 1. In some embodiments, the number of linker-drug moieties per antibody is greater than 4, such as 5, 6, 7, 8, 9, 10, 11, 12 or greater than 12 linker-drug moieties per antibody. Non-limiting examples for determining DAR include various conventional means such as UV spectroscopy, mass spectroscopy, ELISA assay, radiometric methods, hydrophobic interaction chromatography (HIC), electrophoresis and HPLC. In some embodiments, the DAR of an ADC of the disclosure is equivalent to the “n” referred to in formula (I).

In some embodiments, the antibody drug conjugates (ADCs) of the disclosure (e.g., the ADCs of the disclosure comprising antibodies and/or antibody fragments, such as but not limited to CDCP1 antibodies and/or antibody fragments, conjugated to a drug moiety via a linker) are capable of being internalized. In another embodiment, the antibody drug conjugates (ADCs) of the disclosure are capable of inducing cell death of cells endogenously expressing CDCP1.

In some embodiments, L-D has the formula:

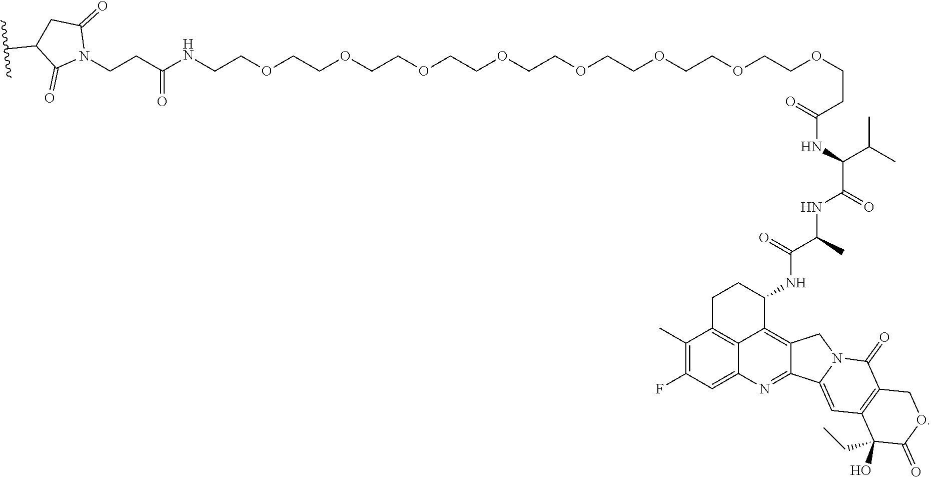 [SMILES: *C1CC(=O)N(CCC(=O)NCCOCCOCCOCCOCCOCCOCCOCCOCCC(=O)N[C@H](C(=O)N[C@@H](C)C(=O)N[C@H]2CCc3c(C)c(F)cc4nc5c(c2c34)Cn2c-5cc3c(c2=O)COC(=O)[C@]3(O)CC)C(C)C)C1=O]

In some embodiments, L-D has the formula:

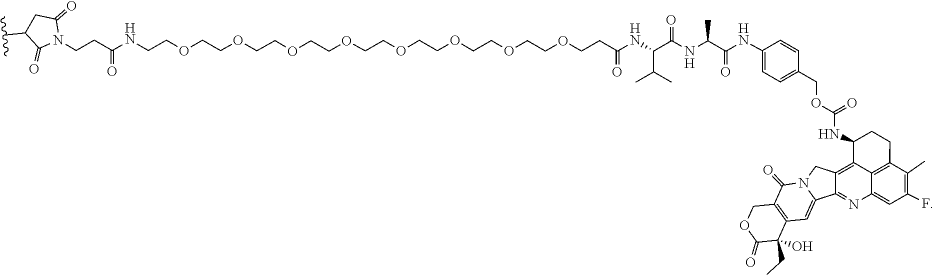 [SMILES: *C1CC(=O)N(CCC(=O)NCCOCCOCCOCCOCCOCCOCCOCCOCCC(=O)N[C@H](C(=O)N[C@@H](C)C(=O)Nc2ccc(COC(=O)N[C@H]3CCc4c(C)c(F)cc5nc6c(c3c45)Cn3c-6cc4c(c3=O)COC(=O)[C@]4(O)CC)cc2)C(C)C)C1=O]

In some embodiments, L-D has the formula:

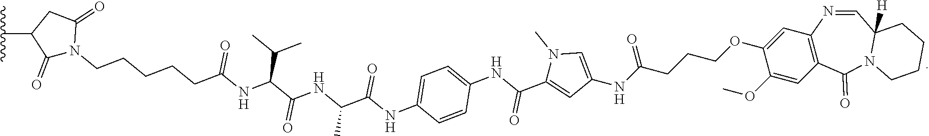 [SMILES: *C1CC(=O)N(CCCCCC(=O)N[C@H](C(=O)N[C@@H](C)C(=O)Nc2ccc(NC(=O)c3cc(NC(=O)CCCOc4cc5c(cc4OC)C(=O)N4CCCC[C@H]4C=N5)cn3C)cc2)C(C)C)C1=O]

In some embodiments, L-D has the formula:

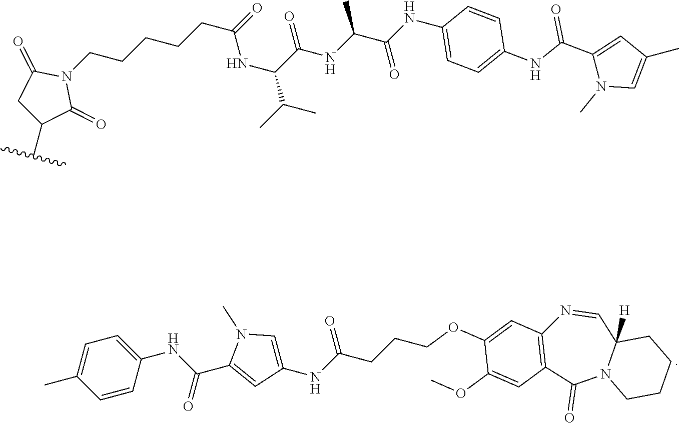

In some embodiments, L-D has the formula:

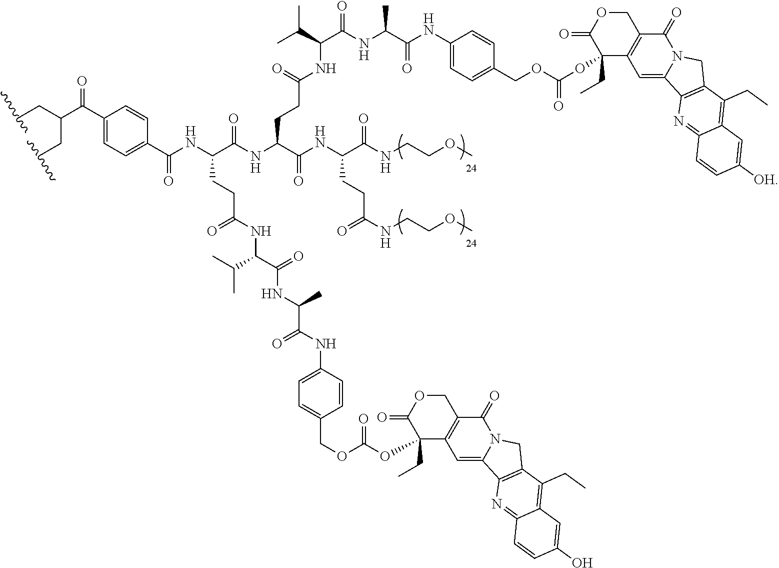

In some embodiments, L-D has the formula:

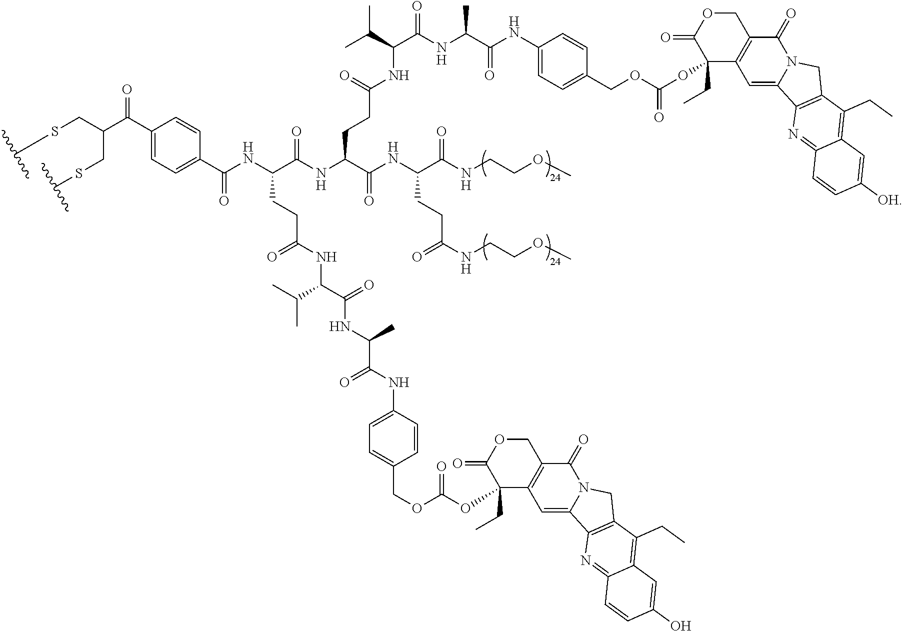

In some embodiments, L-D has the formula:

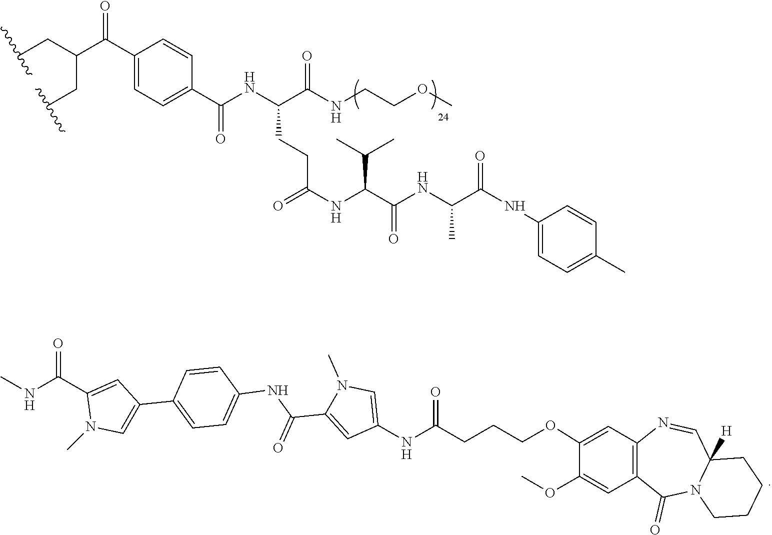

In some embodiments, L-D has the formula:

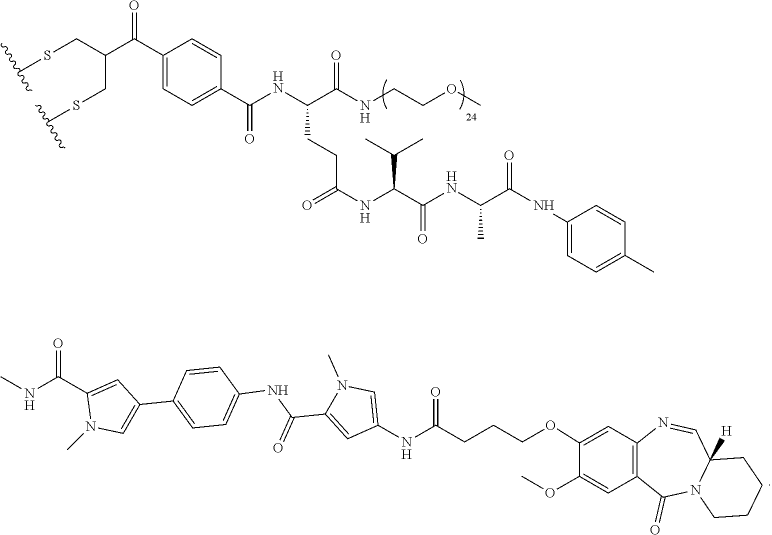

In some embodiments, L-D has the formula -val-cit-MMAE.

In some embodiments, L-D has the formula -val-cit-PAB-MMAE.

In some embodiments, the antibody-drug conjugates described herein (e.g. formula (I)) is capable of forming a metabolite in vivo. In some embodiments, the antibody-drug conjugate is capable of forming a metabolite in vitro.

In some embodiments, the antibody-drug conjugate described herein (e.g. formula (I)) is capable of forming a metabolite of formula 300:

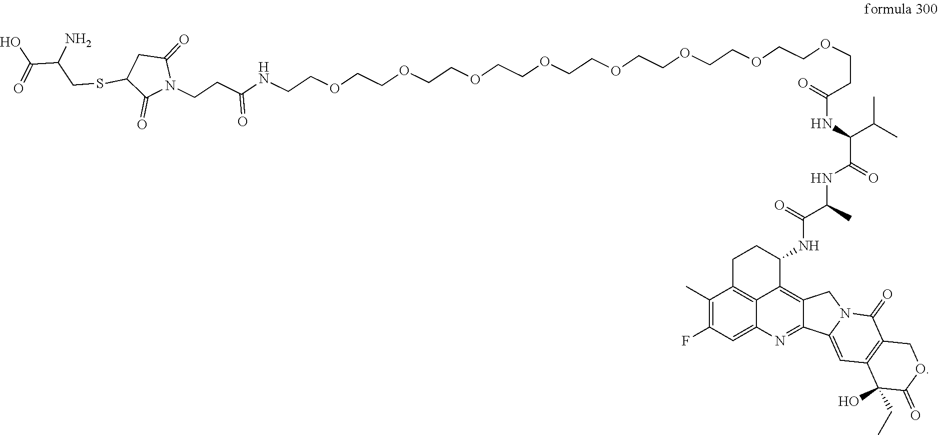 [SMILES: CC[C@@]1(O)C(=O)OCc2c1cc1n(c2=O)Cc2c-1nc1cc(F)c(C)c3c1c2[C@@H](NC(=O)[C@H](C)NC(=O)[C@@H](NC(=O)CCOCCOCCOCCOCCOCCOCCOCCOCCNC(=O)CCN1C(=O)CC(SCC(N)C(=O)O)C1=O)C(C)C)CC3]

In some embodiments, the antibody-drug conjugate described herein (e.g. formula (III)) is capable of forming a metabolite of formula 301:

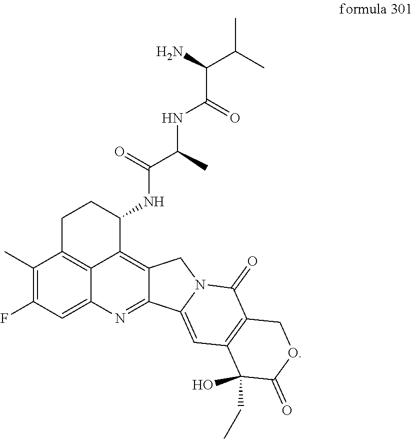 [SMILES: CC[C@@]1(O)C(=O)OCc2c1cc1n(c2=O)Cc2c-1nc1cc(F)c(C)c3c1c2[C@@H](NC(=O)[C@H](C)NC(=O)[C@@H](N)C(C)C)CC3]

In some embodiments, the antibody-drug conjugate described herein (e.g. formula (III)) is capable of forming a metabolite of formula 302:

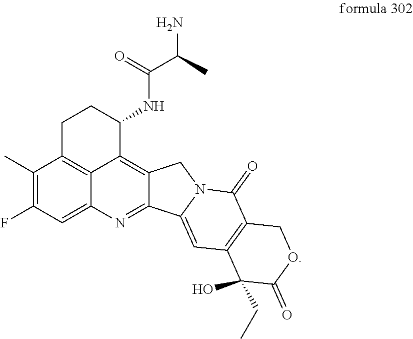 [SMILES: CC[C@@]1(O)C(=O)OCc2c1cc1n(c2=O)Cc2c-1nc1cc(F)c(C)c3c1c2[C@@H](NC(=O)[C@H](C)N)CC3]

In some embodiments, the antibody-drug conjugate described herein (e.g. formula (I)) is capable of forming a metabolite of formula 303:

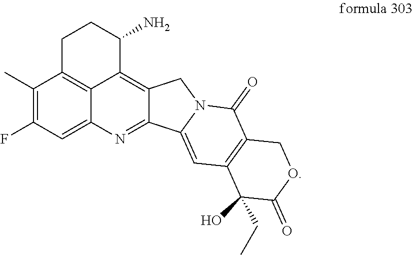 [SMILES: CC[C@@]1(O)C(=O)OCc2c1cc1n(c2=O)Cc2c-1nc1cc(F)c(C)c3c1c2[C@@H](N)CC3]

In some embodiments, the metabolite of formula 300 is capable of forming the metabolite of formula 301. In some embodiments, the metabolite of formula 300 is capable of forming the metabolite of formula 302. In some embodiments, the metabolite of formula 300 is capable of forming the metabolite of formula 303. In some embodiments, the metabolite of formula 301 is capable of forming the metabolite of formula 302. In some embodiments, the metabolite of formula 301 is capable of forming the metabolite of formula 303. In some embodiments, the metabolite of formula 300 is capable of forming the metabolite of formula 301 and the metabolite of formula 301 is capable of forming the metabolite of formula 302. In some embodiments, the metabolite of formula 300 is capable of forming the metabolite of formula 301, the metabolite of formula 301 is capable of forming the metabolite of formula 302, and the metabolite of formula 302 is capable of forming the metabolite of formula 303.

### Methods of Treatment

In one aspect, the conjugates (e.g. ADCs), compounds and compositions described herein (e.g. formula (I), formula (II), and/or formula (III)) can be used in methods for treating diseases. In some embodiments, the disease is cancer. In one embodiment, the disease is a hyperproliferative diseases. In some embodiments, the hyperproliferative disorder is cancer. In some embodiments, the cancer is pancreatic cancer, breast cancer, prostate cancer, lymphoma, skin cancer, colon cancer, melanoma, malignant melanoma, ovarian cancer, brain cancer, primary brain carcinoma, head-neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, chronic lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, or retinoblastoma, and the like. In other embodiments, the cancer is acoustic neuroma, adenocarcinoma, angiosarcoma, astrocytoma, basal cell carcinoma, bile duct carcinoma, bladder carcinoma, brain cancer, breast cancer, triple-negative breast cancer (TNBC), bronchogenic carcinoma, cervical cancer, chordoma, choriocarcinoma, colon cancer, colorectal cancer, craniopharyngioma, cystadenocarcinoma, embryonal carcinoma, endotheliocarcinoma, ependymoma, epithelial carcinoma, esophageal cancer, Ewing's tumor, fibrosarcoma, gastric cancer, glioblastoma multiforme, glioma, head and neck cancer, hemangioblastoma, hepatoma, kidney cancer, leiomyosarcoma, liposarcoma, lung cancer, lymphangioendotheliosarcoma, lymphangiosarcoma, medullary carcinoma, medulloblastoma, melanoma, meningioma, mesothelioma, myxosarcoma, nasal cancer, neuroblastoma, oligodendroglioma, oral cancer, osteogenic sarcoma, ovarian cancer, pancreatic cancer, papillary adenocarcinoma, papillary carcinoma, pinealoma, prostate cancer, rabdomyosarcoma, rectal cancer, renal cell carcinoma, retinoblastoma, sarcoma, sebaceous gland carcinoma, seminoma, skin cancer, squamous cell carcinoma, stomach cancer, sweat gland carcinoma, synovioma, testicular cancer, small cell lung carcinoma, throat cancer, uterine cancer, Wilm's tumor, blood cancer, acute erythroleukemic leukemia, acute lymphoblastic B-cell leukemia, acute lymphoblastic T-cell leukemia, acute lymphoblastic leukemia, acute megakaryoblastic leukemia, acute monoblastic leukemia, acute myeloblastic leukemia, acute myelomonocytic leukemia, acute nonlymphocytic leukemia, acute promyelocytic leukemia, acute undifferentiated leukemia, chronic lymphocytic leukemia, chronic myelocytic leukemia, hairy cell leukemia, multiple myeloma, heavy chain disease, Hodgkin's disease, multiple myeloma, non-Hodgkin's lymphoma, polycythemia vera, or Waldenstrom's macroglobulinemia. In some embodiments, the disease is triple negative breast cancer (TNBC).

In some embodiments, the antibody drug conjugate as described herein reduces mean tumor volume by at least 5%, 10%, 15%, 20%, 25%, 30%, 35%, 40%, 45%, 50%, 55%, 60%, 65%, 70%, 75%, 80%, 85%, 90%, 95%, or 100% as compared to mean tumor volume in untreated controls in a breast cancer MDA-MB-231 model. In some embodiments, the antibody drug conjugate reduces mean tumor volume by at least 5%, 10%, 15%, 20%, 25%, 30%, 35%, 40%, 45%, 50%, 55%, 60%, 65%, 70%, 75%, 80%, 85%, 90%, 95%, or 100% as compared to mean tumor volume in untreated controls in a breast cancer patient-derived xenograft model.

In some embodiments, the method of treating a cancer comprises administering to a subject in need thereof a therapeutically effective amount of an antibody-drug conjugate described herein (e.g. formula (I)) or pharmaceutical composition thereof.

In some embodiments, the antibody-drug conjugate is converted to a metabolite after administering the therapeutically effective amount of the antibody-drug conjugate to the subject.

In some embodiments, less than about 75% of the antibody-drug conjugate is converted to a metabolite about 24 hours after administering the therapeutically effective amount of the antibody-drug conjugate to the subject. In some embodiments, less than about 10%, less than about 15%, less than about 20%, less than about 25%, less than about 30%, less than about 35%, less than about 40%, less than about 45%, less than about 50%, less than about 55%, less than about 60%, less than about 65%, or less than about 70% of the antibody-drug conjugate is converted to a metabolite about 24 hours after administering the therapeutically effective amount of the antibody-drug conjugate to the subject.

In some embodiments, about 10% to less than about 50% of the antibody-drug conjugate is converted to a metabolite about 24 hours after administering the therapeutically effective amount of the antibody-drug conjugate to the subject. In some embodiments, less than about 50% of the antibody-drug conjugate is converted to a metabolite about 24 hours, about 36 hours, about 48 hours, about 60 hours, about 72 hours, or about 84 hours after administering the therapeutically effective amount of the antibody-drug conjugate to the subject. In some embodiments, less than about 50% of the antibody-drug conjugate is converted to a metabolite about 24 hours after administering the therapeutically effective amount of the antibody-drug conjugate to the subject.

In some embodiments, about 10%, about 15%, about 20%, about 25%, about 30%, about 35%, about 40%, about 45%, about 50%, about 55%, about 60%, about 65%, about 70%, or about 75% of the antibody-drug conjugate is converted to a metabolite about 24 hours, about 36 hours, about 48 hours, about 60 hours, about 72 hours, about 84 hours, or about 96 hours after administering the therapeutically effective amount of the antibody-drug conjugate to the subject. In some embodiments, about 10% to about 50% of the antibody-drug conjugate is converted to a metabolite about 36 hours to about 96 hours after administering the therapeutically effective amount of the antibody-drug conjugate to the subject. In some embodiments, about 50% of the antibody-drug conjugate is converted to a metabolite about 36 hours, about 48 hours, about 60 hours, about 72 hours, about 84 hours, or about 96 hours after administering the therapeutically effective amount of the antibody-drug conjugate to the subject. In some embodiments, about 50% of the antibody-drug conjugate is converted to a metabolite about 96 hours after administering the therapeutically effective amount of the antibody-drug conjugate to the subject.

In some embodiments, the antibody-drug conjugate is converted to a metabolite of formula 300 after administering the therapeutically effective amount of the antibody-drug conjugate to the subject:

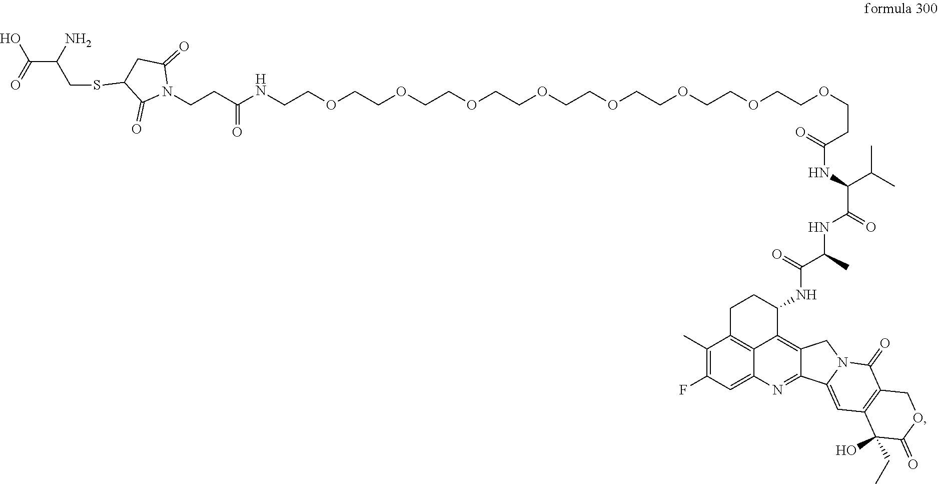 [SMILES: CC[C@@]1(O)C(=O)OCc2c1cc1n(c2=O)Cc2c-1nc1cc(F)c(C)c3c1c2[C@@H](NC(=O)[C@H](C)NC(=O)[C@@H](NC(=O)CCOCCOCCOCCOCCOCCOCCOCCOCCNC(=O)CCN1C(=O)CC(SCC(N)C(=O)O)C1=O)C(C)C)CC3]

In some embodiments, the antibody-drug conjugate is converted to a metabolite of formula 301 after administering the therapeutically effective amount of the antibody-drug conjugate to the subject:

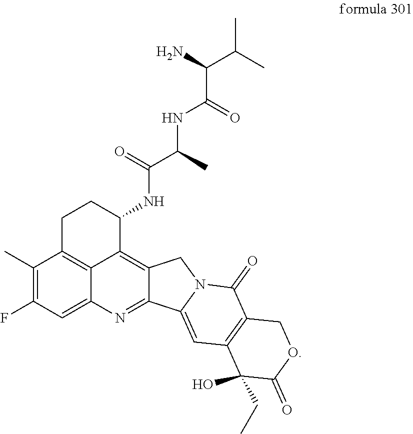 [SMILES: CC[C@@]1(O)C(=O)OCc2c1cc1n(c2=O)Cc2c-1nc1cc(F)c(C)c3c1c2[C@@H](NC(=O)[C@H](C)NC(=O)[C@@H](N)C(C)C)CC3]

In some embodiments, the antibody-drug conjugate is converted to a metabolite of formula 302 after administering the therapeutically effective amount of the antibody-drug conjugate to the subject:

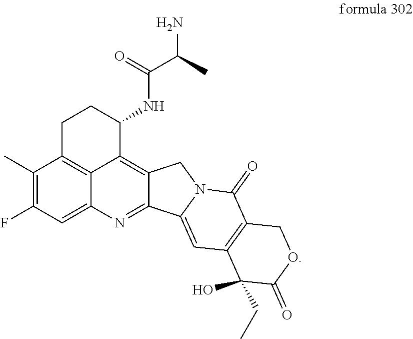 [SMILES: CC[C@@]1(O)C(=O)OCc2c1cc1n(c2=O)Cc2c-1nc1cc(F)c(C)c3c1c2[C@@H](NC(=O)[C@H](C)N)CC3]

In some embodiments, the metabolite of formula 300 is converted to the metabolite of formula 301. In some embodiments, the metabolite of formula 300 is converted to the metabolite of formula 302. In some embodiments, the metabolite of formula 301 is converted to the metabolite of formula 302. In some embodiments, the metabolite of formula 300 is converted to the metabolite of formula 301 and the metabolite of formula 301 is converted to the metabolite of formula 302.

In some embodiments, the antibody-drug conjugate is converted to a metabolite in vivo. In some embodiments, the antibody-drug conjugate is converted to a metabolite in vitro.

In a non-limiting example, the percent of antibody-drug conjugate converted into a metabolite is based on the therapeutically effective amount of the antibody-drug conjugate administered to the subject. Conversion of an antibody-drug conjugate to a metabolite can be determined using any method known in the art. Non-limiting examples of methods useful for determining conversion include mass spectrometry.

### Combination Therapies/Conjugation Agents

As described herein, the present invention relates to, in various embodiments, anti-tumor agents that may be a part of a conjugate and/or compound of the invention or used in the context of various combination therapies encompassed by the present invention.

Combination therapy embraces the administration of an antibody-drug conjugate, and another therapeutic agent as part of a specific treatment regimen, optionally, including a maintenance phase, intended to provide a beneficial effect from the co-action of these therapeutic agents. The beneficial effect of the combination includes, but is not limited to, pharmacokinetic or pharmacodynamic co-action resulting from the combination of therapeutic agents. Administration of these therapeutic agents in combination typically is carried out over a defined time period (usually minutes, hours, days or weeks depending upon the combination selected). Combination therapy generally is not intended to encompass the administration of two or more of these therapeutic agents as part of separate monotherapy regimens that incidentally and arbitrarily result in the combinations of the present invention.

Combination therapy embraces administration of these therapeutic agents in a sequential manner, that is, wherein each therapeutic agent is administered at a different time, as well as administration of these therapeutic agents, or at least two of the therapeutic agents, in a substantially simultaneous manner. Sequential or substantially simultaneous administration of each therapeutic agent can be effected by any appropriate route including, but not limited to, oral routes, intravenous routes, intramuscular, subcutaneous routes, and direct absorption through mucous membrane tissues. The therapeutic agents can be administered by the same route or by different routes. For example, a first therapeutic agent (e.g., a chemotherapeutic agent) can be administered orally, and a second agent (e.g., an ADC) can be administered intravenously. Further, a first therapeutic agent of the combination selected may be administered by intravenous injection while the other therapeutic agents of the combination may be administered orally. Alternatively, for example, both the therapeutic agents may be administered by intravenous or subcutaneous injection.

In the present disclosure the term sequential means, unless otherwise specified, characterized by a regular sequence or order, e.g., if a dosage regimen includes the administration of an ADC and a chemotherapeutic agent, a sequential dosage regimen could include administration of the ADC before, simultaneously, substantially simultaneously, or after administration of the chemotherapeutic agent, but both agents will be administered in a regular sequence or order. The term separate means, unless otherwise specified, to keep apart one from the other. The term simultaneously means, unless otherwise specified, happening or done at the same time, i.e., the compounds of the invention are administered at the same time. The term substantially simultaneously means that the compounds are administered within minutes of each other (e.g., within 10 minutes of each other) and intends to embrace joint administration as well as consecutive administration, but if the administration is consecutive it is separated in time for only a short period (e.g., the time it would take a medical practitioner to administer two compounds separately). As used herein, concurrent administration and substantially simultaneous administration are used interchangeably. Sequential administration refers to temporally separated administration of the ADC and the chemotherapeutic agent.

In some embodiments, the chemotherapeutic agent is selected from alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (e.g., bullatacin and bullatacinone); a camptothecin (including the synthetic analogue topotecan); bryostatin; CC-1065 (including its adozelesin, carzelesin and bizelesin synthetic analogues); cryptophycins (e.g., cryptophycin 1 and cryptophycin 8); dolastatin; duocarmycin (including the synthetic analogues, KW-2189 and CB 1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (see, e.g., Agnew, Chem. Intl. Ed. Engl., 33:183-186 (1994)); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxy doxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as minoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (e.g., T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL paclitaxel, ABRAXANE Cremophor-free, albumin-engineered nanoparticle formulation of paclitaxel (American Pharmaceutical Partners, Schaumberg, 111), and TAXOTERE doxetaxel (Rhone-Poulenc Rorer, Antony, France); chloranbucil; GEMZAR gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE. vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (Tykerb); inhibitors of PKC-α, Raf, H-Ras, EGFR (e.g., erlotinib (Tarceva)) and VEGF-A that reduce cell proliferation and pharmaceutically acceptable salts, acids or derivatives of any of the above.

In some embodiments, the anti-tumor agent is a cytotoxic agent. In some embodiments, the cytotoxic agent is selected from methotrexate, aminopterin, 6-mercaptopurine, 6-thioguanine, cytarabine, 5-fluorouracil decarbazine; alkylating agents such as mechlorethamine, thioepa chlorambucil, melphalan, carmustine (BSNU), mitomycin C, lomustine (CCNU), 1-methylnitrosourea, cyclothosphamide, mechlorethamine, busulfan, dibromomannitol, streptozotocin, mitomycin C, cis-dichlorodiamine platinum (II) (DDP) cisplatin and carboplatin (paraplatin); anthracyclines include daunorubicin, doxorubicin (adriamycin), detorubicin, carminomycin, idarubicin, epirubicin, mitoxantrone and bisantrene; antibiotics include dactinomycin (actinomycin D), bleomycin, calicheamicin, mithramycin, and anthramycin (AMC); and antimytotic agents such as the vinca alkaloids, vincristine and vinblastine, and mixtures thereof.

In some embodiments, the cytotoxic agent is selected from paclitaxel (taxol), ricin, pseudomonas exotoxin, gemcitabine, cytochalasin B, gramicidin D, ethidium bromide, emetine, etoposide, tenoposide, colchicin, dihydroxy anthracin dione, 1-dehydrotestosterone, glucocorticoids, procaine, tetracaine, lidocaine, propranolol, puromycin, procarbazine, hydroxyurea, and mixtures thereof.

In some embodiments, the present compositions and methods find use in combination with checkpoint inhibitors—e.g., in the treatment of various cancers. For instance, the present compositions and methods may supplement checkpoint inhibitor-based cancer therapies, e.g., by improving patient response to the same (e.g., by converting non-responders to responders, and/or increasing the magnitude of therapeutic response, and/or reducing the dose or regimen needed for therapeutic response, and/or reducing one or more side effects of the checkpoint inhibitor-based cancer therapies).

In some embodiments, the checkpoint inhibitor is an agent that targets one of TIM-3, BTLA, PD-1, CTLA-4, B7-H4, GITR, galectin-9, HVEM, PD-L1, PD-L2, B7-H3, CD244, CD160, TIGIT, SIRPα, ICOS, CD172a, and TMIGD2.

In some embodiments, the immune checkpoint immunotherapy agent modulates PD-1) In some embodiments, the agent that targets PD-1 is an antibody or antigen-binding portion thereof that is specific for PD-1, optionally selected from nivolumab, pembrolizumab, and pidilizumab. In some embodiments, an antibody or antigen-binding portion thereof specific for PD-1 is Nivolumab and can be administered at 240 mg every 2 weeks. In some embodiments, an antibody or antigen-binding portion thereof that is specific for PD-1 is Pembrolizumab and can be administered at 200 mg every 3 weeks. In some embodiments, an antibody or antigen-binding portion thereof that is specific for PD-1 is Pidilizumab and can be administered at 200 mg every 3 weeks.

In some embodiments, the immune checkpoint immunotherapy agent modulates PD-L1. In some embodiments, the agent that modulates PD-L1 is an antibody or antigen-binding portion thereof that is specific for PD-L1. In some embodiments, the antibody or antigen-binding portion thereof that is specific for PD-L1 is selected from Atezolizumab, Avelumab, Durvalumab, and BMS-936559. In some embodiments, the antibody or antigen-binding portion thereof that is specific for PD-L1 is BMS-936559 and can be administered at 0.1 mg/kg every 2 weeks. In some embodiments, the antibody or antigen-binding portion thereof that is specific for PD-L1 is Atezolizumab and can be administered at 1200 mg every 3 weeks. In some embodiments, the antibody or antigen-binding portion thereof that is specific for PD-L1 is Avelumab and can be administered at 10 mg/kg every 2 weeks. In some embodiments, the antibody or antigen-binding portion thereof that is specific for PD-L1 is Durvalumab and can be administered at 10 mg/kg every 2 weeks.

In some embodiments, the agent that targets CTLA-4 is an antibody or antigen-binding portion thereof that is specific for CTLA-4, optionally selected from ipilimumab and tremelimumab. In some embodiments, the antibody or antigen-binding portion thereof that is specific for CTLA-4 is tremelimumab and can administered at 3 mg/kg, 6 mg/kg or 10 mg/kg. In some embodiments, the antibody or antigen-binding portion thereof that is specific for CTLA-4 is Ipilimumab and can administered at 5 mg/mL 12 weeks.

In some embodiments, the hyperproliferative disorder (e.g., cancer) treated by the compounds and compositions described herein includes cells having p38α MAPK protein and/or p38α MAPK related protein expression.

In one embodiment, the disclosure relates to a method of treating a disease alleviated by inhibiting the p38α MAPK protein in a patient in need thereof, including administering to the patient a therapeutically effective amount of a p38α MAPK inhibitor, wherein the p38α MAPK inhibitor is a compound capable of binding to a pocket near the ED substrate-docking site of p38α MAPK, or a pharmaceutically acceptable salt, solvate, hydrate, cocrystal, or prodrug thereof, and one or more additional therapeutic agents, including chemotherapeutic and/or immunotherapeutic agents.

Efficacy of the compounds and combinations of compounds described herein in treating the indicated diseases or disorders can be tested using various models known in the art, and described herein, which provide guidance for treatment of human disease. Any and all of the described methods of treatment may include medical follow-up to determine the therapeutic or prophylactic effect brought about in the subject undergoing treatment with the compound(s) and/or composition(s) described herein.

### Pharmaceutical Compositions

In an embodiment, an active pharmaceutical ingredient or combination of active pharmaceutical ingredients, such as any of the conjugates, drug moieties, linkers, compounds, and/or compositions of the disclosure, is provided as a pharmaceutically acceptable composition.

In one embodiment, the disclosure relates to a pharmaceutical composition including a therapeutically effective amount of one or more conjugates, drug moieties, linkers, compounds, and/or compositions of the disclosure (e.g. formula (I) formula (II), and/or formula (III)), or a pharmaceutically acceptable salt, solvate, hydrate, cocrystal, or prodrug thereof; and a physiologically compatible carrier medium, wherein the disease is cancer. In one embodiment, the diseases is a cancer such as acoustic neuroma, adenocarcinoma, angiosarcoma, astrocytoma, basal cell carcinoma, bile duct carcinoma, bladder carcinoma, brain cancer, breast cancer, bronchogenic carcinoma, cervical cancer, chordoma, choriocarcinoma, colon cancer, colorectal cancer, craniopharyngioma, cystadenocarcinoma, embryonal carcinoma, endotheliocarcinoma, ependymoma, epithelial carcinoma, esophageal cancer, Ewing's tumor, fibrosarcoma, gastric cancer, glioblastoma multiforme, glioma, head and neck cancer, hemangioblastoma, hepatoma, kidney cancer, leiomyosarcoma, liposarcoma, lung cancer, lymphangioendotheliosarcoma, lymphangiosarcoma, medullary carcinoma, medulloblastoma, melanoma, meningioma, mesothelioma, myxosarcoma, nasal cancer, neuroblastoma, oligodendroglioma, oral cancer, osteogenic sarcoma, ovarian cancer, pancreatic cancer, papillary adenocarcinoma, papillary carcinoma, pinealoma, prostate cancer, rabdomyosarcoma, rectal cancer, renal cell carcinoma, retinoblastoma, sarcoma, sebaceous gland carcinoma, seminoma, skin cancer, squamous cell carcinoma, stomach cancer, sweat gland carcinoma, synovioma, testicular cancer, small cell lung carcinoma, throat cancer, triple-negative breast cancer (TNBC), uterine cancer, Wilm's tumor, blood cancer, acute erythroleukemic leukemia, acute lymphoblastic B-cell leukemia, acute lymphoblastic T-cell leukemia, acute lymphoblastic leukemia, acute megakaryoblastic leukemia, acute monoblastic leukemia, acute myeloblastic leukemia, acute myelomonocytic leukemia, acute nonlymphocytic leukemia, acute promyelocytic leukemia, acute undifferentiated leukemia, chronic lymphocytic leukemia, chronic myelocytic leukemia, hairy cell leukemia, multiple myeloma, heavy chain disease, Hodgkin's disease, multiple myeloma, non-Hodgkin's lymphoma, polycythemia vera, or Waldenstrom's macroglobulinemia. In some embodiments, the disease is triple-negative breast cancer (TNBC).

In some embodiments, the concentration of each of the active pharmaceutical ingredients provided in the pharmaceutical compositions of the disclosure, such as any of the conjugates, drug moieties, linkers, compounds, and/or compositions of the disclosure (e.g. formula (I) formula (II), and/or formula (III)), is less than, for example, 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v, or v/v of the pharmaceutical composition.

In some embodiments, the concentration of each of the active pharmaceutical ingredients provided in the pharmaceutical compositions of the disclosure, such as any of the conjugates, drug moieties, linkers, compounds, and/or compositions of the disclosure (e.g. formula (I) formula (II), and/or formula (III)), is greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v, or v/v of the pharmaceutical composition.

In some embodiments, the concentration of each of the active pharmaceutical ingredients provided in the pharmaceutical compositions of the disclosure, such as any of the conjugates, drug moieties, linkers, compounds, and/or compositions of the disclosure (e.g. formula (I) formula (II), and/or formula (III)), is in the range from about 0.0001% to about 50%, about 0.001% to about 40%, about 0.01% to about 30%, about 0.02% to about 29%, about 0.03% to about 28%, about 0.04% to about 27%, about 0.05% to about 26%, about 0.06% to about 25%, about 0.07% to about 24%, about 0.08% to about 23%, about 0.09% to about 22%, about 0.1% to about 21%, about 0.2% to about 20%, about 0.3% to about 19%, about 0.4% to about 18%, about 0.5% to about 17%, about 0.6% to about 16%, about 0.7% to about 15%, about 0.8% to about 14%, about 0.9% to about 12% or about 1% to about 10% w/w, w/v, or v/v of the pharmaceutical composition.

In some embodiments, the concentration of each of the active pharmaceutical ingredients provided in the pharmaceutical compositions of the disclosure, such as any of the conjugates, drug moieties, linkers, compounds, and/or compositions of the disclosure (e.g. formula (I) formula (II), and/or formula (III)), is in the range from about 0.001% to about 10%, about 0.01% to about 5%, about 0.02% to about 4.5%, about 0.03% to about 4%, about 0.04% to about 3.5%, about 0.05% to about 3%, about 0.06% to about 2.5%, about 0.07% to about 2%, about 0.08% to about 1.5%, about 0.09% to about 1%, about 0.1% to about 0.9% w/w, w/v, or v/v of the pharmaceutical composition.

In some embodiments, the amount of each of the active pharmaceutical ingredients provided in the pharmaceutical compositions of the disclosure, such as any of the foregoing conjugates, drug moieties, linkers, compounds, and/or compositions of the disclosure (e.g. formula (I) formula (II), and/or formula (III)), is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of each of the active pharmaceutical ingredients provided in the pharmaceutical compositions of the disclosure, such as any of the conjugates, drug moieties, linkers, compounds, and/or compositions of the disclosure (e.g. formula (I) formula (II), and/or formula (III)), is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g.

Each of the active pharmaceutical ingredients according to the disclosure is effective over a wide dosage range. For example, in the treatment of adult humans, dosages independently range from 0.01 to 1000 mg, from 0.5 to 100 mg, from 1 to 50 mg per day, and from 5 to 40 mg per day are examples of dosages that may be used. The exact dosage will depend upon the route of administration, the form in which the compound is administered, the gender and age of the subject to be treated, the body weight of the subject to be treated, and the preference and experience of the attending physician. The clinically-established dosages of the conjugates, drug moieties, linkers, compounds, and/or compositions of the disclosure (e.g. formula (I) formula (II), and/or formula (III)), of the disclosure may also be used if appropriate.

In an embodiment, the molar ratio of two active pharmaceutical ingredients in the pharmaceutical compositions is in the range from 10:1 to 1:10, preferably from 2.5:1 to 1:2.5, and more preferably about 1:1. In an embodiment, the weight ratio of the molar ratio of two active pharmaceutical ingredients in the pharmaceutical compositions is selected from the group consisting of 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, and 1:20. In an embodiment, the weight ratio of the molar ratio of two active pharmaceutical ingredients in the pharmaceutical compositions is selected from the group consisting of 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, and 1:20.

Described below are non-limiting pharmaceutical compositions and methods for preparing the same.

### Pharmaceutical Compositions for Oral Administration

In an embodiment, the disclosure provides a pharmaceutical composition for oral administration containing the active pharmaceutical ingredient or combination of active pharmaceutical ingredients, such as the conjugates, drug moieties, linkers, compounds, and/or compositions of the disclosure (e.g. formula (I) formula (II), and/or formula (III)), described herein, and a pharmaceutical excipient suitable for oral administration.

In some embodiments, the disclosure provides a solid pharmaceutical composition for oral administration containing: (i) an effective amount of an active pharmaceutical ingredient or combination of active pharmaceutical ingredients, and (ii) a pharmaceutical excipient suitable for oral administration. In selected embodiments, the composition further contains (iii) an effective amount of a third active pharmaceutical ingredient, and optionally (iv) an effective amount of a fourth active pharmaceutical ingredient.

In some embodiments, the pharmaceutical composition may be a liquid pharmaceutical composition suitable for oral consumption. Pharmaceutical compositions of the disclosure suitable for oral administration can be presented as discrete dosage forms, such as capsules, sachets, or tablets, or liquids or aerosol sprays each containing a predetermined amount of an active ingredient as a powder or in granules, a solution, or a suspension in an aqueous or non-aqueous liquid, an oil-in-water emulsion, a water-in-oil liquid emulsion, powders for reconstitution, powders for oral consumptions, bottles (including powders or liquids in a bottle), orally dissolving films, lozenges, pastes, tubes, gums, and packs. Such dosage forms can be prepared by any of the methods of pharmacy, but all methods include the step of bringing the active ingredient(s) into association with the carrier, which constitutes one or more necessary ingredients. In general, the compositions are prepared by uniformly and intimately admixing the active ingredient(s) with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

The disclosure further encompasses anhydrous pharmaceutical compositions and dosage forms since water can facilitate the degradation of some compounds. For example, water may be added (e.g., 5%) in the pharmaceutical arts as a means of simulating long-term storage in order to determine characteristics such as shelf-life or the stability of formulations over time. Anhydrous pharmaceutical compositions and dosage forms of the disclosure can be prepared using anhydrous or low moisture containing ingredients and low moisture or low humidity conditions. Pharmaceutical compositions and dosage forms of the disclosure which contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

Each of the active pharmaceutical ingredients can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants may be used in the compositions of the disclosure to provide tablets that disintegrate when exposed to an aqueous environment. Too much of a disintegrant may produce tablets which disintegrate in the bottle. Too little may be insufficient for disintegration to occur, thus altering the rate and extent of release of the active ingredients from the dosage form. Thus, a sufficient amount of disintegrant that is neither too little nor too much to detrimentally alter the release of the active ingredient(s) may be used to form the dosage forms of the compounds disclosed herein. The amount of disintegrant used may vary based upon the type of formulation and mode of administration, and may be readily discernible to those of ordinary skill in the art. About 0.5 to about 15 weight percent of disintegrant, or about 1 to about 5 weight percent of disintegrant, may be used in the pharmaceutical composition. Disintegrants that can be used to form pharmaceutical compositions and dosage forms of the disclosure include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions and dosage forms of the disclosure include, but are not limited to, calcium stearate, magnesium stearate, sodium stearyl fumarate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethylaureate, agar, or mixtures thereof. Additional lubricants include, for example, a syloid silica gel, a coagulated aerosol of synthetic silica, silicified microcrystalline cellulose, or mixtures thereof. A lubricant can optionally be added in an amount of less than about 0.5% or less than about 1% (by weight) of the pharmaceutical composition.

When aqueous suspensions and/or elixirs are desired for oral administration, the active pharmaceutical ingredient(s) may be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactants which can be used to form pharmaceutical compositions and dosage forms of the disclosure include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions. Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acyl-lactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogs thereof; polyoxyethylated vitamins and derivatives thereof; polyoxyethylene-polyoxypropylene block copolymers; and mixtures thereof; polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10 oleate, Tween 40, Tween 60, sucrose monostearate, sucrose monolaurate, sucrose monopalmitate, PEG 10−100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In an embodiment, the composition may include a solubilizer to ensure good solubilization and/or dissolution of the compound of the present disclosure and to minimize precipitation of the compound of the present disclosure. This can be especially important for compositions for non-oral use—e.g., compositions for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, ε-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, ε-caprolactone and isomers thereof, δ-valerolactone and isomers thereof, β-butyrolactone and isomers thereof; and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a patient using conventional techniques, such as distillation or evaporation. Thus, if present, the solubilizer can be in a weight ratio of 10%, 25%, 50%, 100%, or up to about 200% by weight, based on the combined weight of the drug, and other excipients. If desired, very small amounts of solubilizer may also be used, such as 5%, 2%, 1% or even less. Typically, the solubilizer may be present in an amount of about 1% to about 100%, more typically about 5% to about 25% by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals and alkaline earth metals. Example may include, but not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid and uric acid.

### Pharmaceutical Compositions for Injection

In some embodiments, a pharmaceutical composition is provided for injection containing an active pharmaceutical ingredient or combination of active pharmaceutical ingredients, such as a conjugate, drug moiety, linker, compounds, and/or compositions of the disclosure (e.g. formula (I), formula (II), and/or formula (III)), and a pharmaceutical excipient suitable for injection.

The forms in which the compositions of the present disclosure may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol and liquid polyethylene glycol (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid, and thimerosal.

Sterile injectable solutions are prepared by incorporating an active pharmaceutical ingredient or combination of active pharmaceutical ingredients in the required amounts in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

### Pharmaceutical Compositions for Topical Delivery

In some embodiments, a pharmaceutical composition is provided for transdermal delivery containing an active pharmaceutical ingredient or combination of active pharmaceutical ingredients, such as conjugates, drug moieties, linkers, compounds, and/or compositions of the disclosure (e.g. formula (I), formula (II), and/or formula (III)), and a pharmaceutical excipient suitable for transdermal delivery.

Compositions of the present disclosure can be formulated into preparations in solid, semi-solid, or liquid forms suitable for local or topical administration, such as gels, water soluble jellies, creams, lotions, suspensions, foams, powders, slurries, ointments, solutions, oils, pastes, suppositories, sprays, emulsions, saline solutions, dimethylsulfoxide (DMSO)-based solutions. In general, carriers with higher densities are capable of providing an area with a prolonged exposure to the active ingredients. In contrast, a solution formulation may provide more immediate exposure of the active ingredient to the chosen area.

The pharmaceutical compositions also may comprise suitable solid or gel phase carriers or excipients, which are compounds that allow increased penetration of, or assist in the delivery of, therapeutic molecules across the stratum corneum permeability barrier of the skin. There are many of these penetration-enhancing molecules known to those trained in the art of topical formulation. Examples of such carriers and excipients include, but are not limited to, humectants (e.g., urea), glycols (e.g., propylene glycol), alcohols (e.g., ethanol), fatty acids (e.g., oleic acid), surfactants (e.g., isopropyl myristate and sodium lauryl sulfate), pyrrolidones, glycerol monolaurate, sulfoxides, terpenes (e.g., menthol), amines, amides, alkanes, alkanols, water, calcium carbonate, calcium phosphate, various sugars, starches, cellulose derivatives, gelatin, and polymers such as polyethylene glycols.

Another exemplary formulation for use in the methods of the present disclosure employs transdermal delivery devices (“patches”). Such transdermal patches may be used to provide continuous or discontinuous infusion of an active pharmaceutical ingredient or combination of active pharmaceutical ingredients in controlled amounts, either with or without another active pharmaceutical ingredient.

The construction and use of transdermal patches for the delivery of pharmaceutical agents is well known in the art. See, e.g., U.S. Pat. Nos. 5,023,252; 4,992,445; and 5,001,139, the entirety of which are incorporated herein by reference. Such patches may be constructed for continuous, pulsatile, or on demand delivery of pharmaceutical agents.

### Pharmaceutical Compositions for Inhalation

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra and the conjugates, drug moieties, linkers, compounds, and/or compositions of the disclosure (e.g. formula (I), formula (II), and/or formula (III)). Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices that deliver the formulation in an appropriate manner. Dry powder inhalers may also be used to provide inhaled delivery of the compositions.

### Other Pharmaceutical Compositions

Pharmaceutical compositions of the conjugates, drug moieties, linkers, compounds, and/or compositions of the disclosure (e.g. formula (I), formula (II), and/or formula (III)), may also be prepared from compositions described herein and one or more pharmaceutically acceptable excipients suitable for sublingual, buccal, rectal, intraosseous, intraocular, intranasal, epidural, or intraspinal administration. Preparations for such pharmaceutical compositions are well-known in the art. See, e.g., Anderson, Philip O.; Knoben, James E.; Troutman, William G, eds., Handbook of Clinical Drug Data, Tenth Edition, McGraw-Hill, 2002; and Pratt and Taylor, eds., Principles of Drug Action, Third Edition, Churchill Livingston, N.Y., 1990, each of which is incorporated by reference herein in its entirety.

Administration of an active pharmaceutical ingredient or combination of active pharmaceutical ingredients or a pharmaceutical composition thereof can be effected by any method that enables delivery of the compounds to the site of action. These methods include oral routes, intraduodenal routes, parenteral injection (including intravenous, intraarterial, subcutaneous, intramuscular, intravascular, intraperitoneal or infusion), topical (e.g., transdermal application), rectal administration, via local delivery by catheter or stent or through inhalation. The active pharmaceutical ingredient or combination of active pharmaceutical ingredients can also be administered intraadiposally or intrathecally.

Exemplary parenteral administration forms include solutions or suspensions of active compound in sterile aqueous solutions, for example, aqueous propylene glycol or dextrose solutions. Such dosage forms can be suitably buffered, if desired.

### Kits

The disclosure also provides kits. The kits include an active pharmaceutical ingredient or combination of active pharmaceutical ingredients, either alone or in combination in suitable packaging, and written material that can include instructions for use, discussion of clinical studies and listing of side effects. Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. The kit may further contain another active pharmaceutical ingredient. In selected embodiments, an active pharmaceutical ingredient or combination of active pharmaceutical ingredients are provided as separate compositions in separate containers within the kit. In selected embodiments, an active pharmaceutical ingredient or combination of active pharmaceutical ingredients are provided as a single composition within a container in the kit. Suitable packaging and additional articles for use (e.g., measuring cup for liquid preparations, foil wrapping to minimize exposure to air, and the like) are known in the art and may be included in the kit. Kits described herein can be provided, marketed and/or promoted to health providers, including physicians, nurses, pharmacists, formulary officials, and the like. Kits may also, in selected embodiments, be marketed directly to the consumer.

In some embodiments, the disclosure provides a kit comprising a composition comprising a therapeutically effective amount of an active pharmaceutical ingredient (e.g., a conjugates, drug moieties, linkers, compounds, and/or compositions of the disclosure (e.g. formula (I), formula (II), and/or formula (III)), or combination of active pharmaceutical ingredients or a pharmaceutically acceptable salt, solvate, hydrate, cocrystal, or prodrug thereof. These compositions are typically pharmaceutical compositions. The kit is for co-administration of the active pharmaceutical ingredient or combination of active pharmaceutical ingredients, either simultaneously or separately.

In some embodiments, the disclosure provides a kit comprising (1) a composition comprising a therapeutically effective amount of an active pharmaceutical ingredient (e.g., a conjugates, drug moieties, linkers, compounds, and/or compositions of the disclosure (e.g. formula (I), formula (II), and/or formula (III)), or combination of active pharmaceutical ingredients or a pharmaceutically acceptable salt, solvate, hydrate, cocrystal, or prodrug thereof, and (2) a diagnostic test for determining whether a patient's cancer is a particular subtype of a cancer. Any of the foregoing diagnostic methods may be utilized in the kit.

The kits described above are preferably for use in the treatment of the diseases and conditions described herein. In some embodiments, the kits are for use in the treatment of hyperproliferative disorders, such as cancer.

In a particular embodiment, the kits described herein are for use in the treatment of cancer. In some embodiments, the kits described herein are for use in the treatment of a cancer selected from the group consisting of acoustic neuroma, adenocarcinoma, angiosarcoma, astrocytoma, basal cell carcinoma, bile duct carcinoma, bladder carcinoma, brain cancer, breast cancer, bronchogenic carcinoma, cervical cancer, chordoma, choriocarcinoma, colon cancer, colorectal cancer, craniopharyngioma, cystadenocarcinoma, embryonal carcinoma, endotheliocarcinoma, ependymoma, epithelial carcinoma, esophageal cancer, Ewing's tumor, fibrosarcoma, gastric cancer, glioblastoma multiforme, glioma, head and neck cancer, hemangioblastoma, hepatoma, kidney cancer, leiomyosarcoma, liposarcoma, lung cancer, lymphangioendotheliosarcoma, lymphangiosarcoma, medullary carcinoma, medulloblastoma, melanoma, meningioma, mesothelioma, myxosarcoma, nasal cancer, neuroblastoma, oligodendroglioma, oral cancer, osteogenic sarcoma, ovarian cancer, pancreatic cancer, papillary adenocarcinoma, papillary carcinoma, pinealoma, prostate cancer, rabdomyosarcoma, rectal cancer, renal cell carcinoma, retinoblastoma, sarcoma, sebaceous gland carcinoma, seminoma, skin cancer, squamous cell carcinoma, stomach cancer, sweat gland carcinoma, synovioma, testicular cancer, small cell lung carcinoma, throat cancer, triple-negative breast cancer (TNBC), uterine cancer, Wilm's tumor, blood cancer, acute erythroleukemic leukemia, acute lymphoblastic B-cell leukemia, acute lymphoblastic T-cell leukemia, acute lymphoblastic leukemia, acute megakaryoblastic leukemia, acute monoblastic leukemia, acute myeloblastic leukemia, acute myelomonocytic leukemia, acute nonlymphocytic leukemia, acute promyelocytic leukemia, acute undifferentiated leukemia, chronic lymphocytic leukemia, chronic myelocytic leukemia, hairy cell leukemia, multiple myeloma, heavy chain disease, Hodgkin's disease, multiple myeloma, non-Hodgkin's lymphoma, polycythemia vera, or Waldenstrom's macroglobulinemia. In some embodiments, the disease is triple-negative breast cancer (TNBC).

### Dosages and Dosing Regimens

The amounts of the pharmaceutical compositions administered using the methods herein, such as the dosages of conjugates, drug moieties, linkers, compounds, and/or compositions of the disclosure (e.g. formula (I), formula (II), and/or formula (III)), will be dependent on the human or mammal being treated, the severity of the disorder or condition, the rate of administration, the disposition of the active pharmaceutical ingredients and the discretion of the prescribing physician. However, an effective dosage is in the range of about 0.001 to about 100 mg per kg body weight per day, such as about 1 to about 35 mg/kg/day, in single or divided doses. For a 70 kg human, this would amount to about 0.05 to 7 g/day, such as about 0.05 to about 2.5 g/day. In some instances, dosage levels below the lower limit of the aforesaid range may be more than adequate, while in other cases still larger doses may be employed without causing any harmful side effect—e.g., by dividing such larger doses into several small doses for administration throughout the day. The dosage of the pharmaceutical compositions and active pharmaceutical ingredients may be provided in units of mg/kg of body mass or in mg/m² of body surface area.

In some embodiments, a pharmaceutical composition or active pharmaceutical ingredient is administered in a single dose. Such administration may be by injection, e.g., intravenous injection, in order to introduce the active pharmaceutical ingredient quickly. However, other routes, including the preferred oral route, may be used as appropriate. A single dose of a pharmaceutical composition may also be used for treatment of an acute condition.

In some embodiments, a pharmaceutical composition or active pharmaceutical ingredient is administered in multiple doses. In an embodiment, a pharmaceutical composition is administered in multiple doses. Dosing may be once, twice, three times, four times, five times, six times, or more than six times per day. Dosing may be once a month, once every two weeks, once a week, or once every other day. In other embodiments, a pharmaceutical composition is administered about once per day to about 6 times per day. In some embodiments, a pharmaceutical composition is administered once daily, while in other embodiments, a pharmaceutical composition is administered twice daily, and in other embodiments a pharmaceutical composition is administered three times daily.

Administration of the active pharmaceutical ingredients may continue as long as necessary. In selected embodiments, a pharmaceutical composition is administered for more than 1, 2, 3, 4, 5, 6, 7, 14, or 28 day(s). In some embodiments, a pharmaceutical composition is administered for less than 28, 14, 7, 6, 5, 4, 3, 2, or 1 day(s). In some embodiments, a pharmaceutical composition is administered chronically on an ongoing basis—e.g., for the treatment of chronic effects. In some embodiments, the administration of a pharmaceutical composition continues for less than about 7 days. In yet another embodiment the administration continues for more than about 6, 10, 14, 28 days, two months, six months, or one year. In some cases, continuous dosing is achieved and maintained as long as necessary.

In some embodiments, an effective dosage of an active pharmaceutical ingredient disclosed herein, for example any of the conjugates, drug moieties, linkers, compounds, and/or compositions of the disclosure (e.g. formula (I), formula (II), and/or formula (III)), is in the range of about 1 mg to about 500 mg, about 10 mg to about 300 mg, about 20 mg to about 250 mg, about 25 mg to about 200 mg, about 10 mg to about 200 mg, about 20 mg to about 150 mg, about 30 mg to about 120 mg, about 10 mg to about 90 mg, about 20 mg to about 80 mg, about 30 mg to about 70 mg, about 40 mg to about 60 mg, about 45 mg to about 55 mg, about 48 mg to about 52 mg, about 50 mg to about 150 mg, about 60 mg to about 140 mg, about 70 mg to about 130 mg, about 80 mg to about 120 mg, about 90 mg to about 110 mg, about 95 mg to about 105 mg, about 150 mg to about 250 mg, about 160 mg to about 240 mg, about 170 mg to about 230 mg, about 180 mg to about 220 mg, about 190 mg to about 210 mg, about 195 mg to about 205 mg, or about 198 to about 202 mg. In some embodiments, an effective dosage of an active pharmaceutical ingredient disclosed herein is less than about 25 mg, less than about 50 mg, less than about 75 mg, less than about 100 mg, less than about 125 mg, less than about 150 mg, less than about 175 mg, less than about 200 mg, less than about 225 mg, or less than about 250 mg. In some embodiments, an effective dosage of an active pharmaceutical ingredient disclosed herein is greater than about 25 mg, greater than about 50 mg, greater than about 75 mg, greater than about 100 mg, greater than about 125 mg, greater than about 150 mg, greater than about 175 mg, greater than about 200 mg, greater than about 225 mg, or greater than about 250 mg.

In some embodiments, an effective dosage of an active pharmaceutical ingredient disclosed herein, for example any of the conjugates, drug moieties, linkers, compounds, and/or compositions of the disclosure (e.g. formula (I), formula (II), and/or formula (III)), is in the range of about 0.01 mg/kg to about 200 mg/kg, or about 0.1 to 100 mg/kg, or about 1 to 50 mg/kg.

In some embodiments, an active pharmaceutical ingredient is administered at a dosage of 10 to 200 mg BID, including 50, 60, 70, 80, 90, 100, 150, or 200 mg BID. In some embodiments, an active pharmaceutical ingredient is administered at a dosage of 10 to 500 mg BID, including 1, 5, 10, 15, 25, 50, 75, 100, 150, 200, 300, 400, or 500 mg BID.

In some instances, dosage levels below the lower limit of the aforesaid ranges may be more than adequate, while in other cases still larger doses may be employed without causing any harmful side effect, e.g., by dividing such larger doses into several small doses for administration throughout the day. Of course, as those skilled in the art will appreciate, the dosage actually administered will depend upon the condition being treated, the age, health and weight of the recipient, the type of concurrent treatment, if any, and the frequency of treatment. Moreover, the effective dosage amount may be determined by one skilled in the art on the basis of routine empirical activity testing to measure the bioactivity of the compound(s) in a bioassay, and thus establish the appropriate dosage to be administered.

An effective amount of the combination of the active pharmaceutical ingredient may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, or as an inhalant.

In some embodiments, the compositions described herein further include controlled-release, sustained release, or extended-release therapeutic dosage forms for administration of the compounds described herein, which involves incorporation of the compounds into a suitable delivery system in the formation of certain compositions. This dosage form controls release of the compound(s) in such a manner that an effective concentration of the compound(s) in the bloodstream may be maintained over an extended period of time, with the concentration in the blood remaining relatively constant, to improve therapeutic results and/or minimize side effects. Additionally, a controlled-release system would provide minimum peak to trough fluctuations in blood plasma levels of the compound.

### Numbered Embodiments

The disclosure will be further described in the following embodiments, which do not limit the scope of the disclosure described in the claims.

Embodiment 1. A conjugate of formula (II):

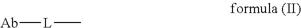

wherein A is a targeting moiety, optionally an antibody or antibody fragment, L is a linker.

Embodiment 2. The conjugate of embodiment 1, optionally further comprising a drug moiety wherein the drug moiety is selected from exatecan, Dxd, Sn-38, monomethyl auristatin E (MMAE), and a pyrridinobenzodiazepine (PDD).

Embodiment 3. The conjugate of embodiment 1 or 2, wherein the exatecan comprises or has the formula:

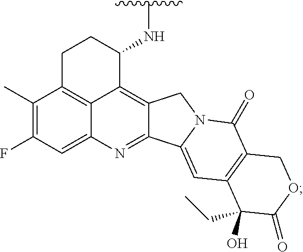 [SMILES: *N[C@H]1CCc2c(C)c(F)cc3nc4c(c1c23)Cn1c-4cc2c(c1=O)COC(=O)[C@]2(O)CC]

or wherein the PDD comprises or has the formula:

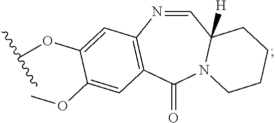 [SMILES: *Oc1cc2c(cc1OC)C(=O)N1CCCC[C@H]1C=N2]

or wherein the PDD comprises or has the formula:

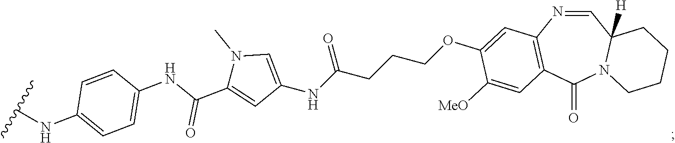 [SMILES: *Nc1ccc(NC(=O)c2cc(NC(=O)CCCOc3cc4c(cc3OC)C(=O)N3CCCC[C@H]3C=N4)cn2C)cc1]

or wherein the PDD comprises or has the formula:

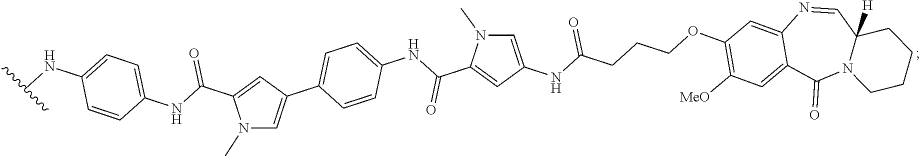 [SMILES: *Nc1ccc(NC(=O)c2cc(-c3ccc(NC(=O)c4cc(NC(=O)CCCOc5cc6c(cc5OC)C(=O)N5CCCC[C@H]5C=N6)cn4C)cc3)cn2C)cc1]

or wherein the Sn-38 comprises or has the formula:

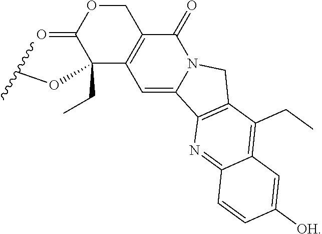 [SMILES: *O[C@]1(CC)C(=O)OCc2c1cc1n(c2=O)Cc2c-1nc1ccc(O)cc1c2CC]

Embodiment 3a: The conjugate of embodiment 1 or embodiment 2, wherein the conjugate further comprises a drug moiety selected from:

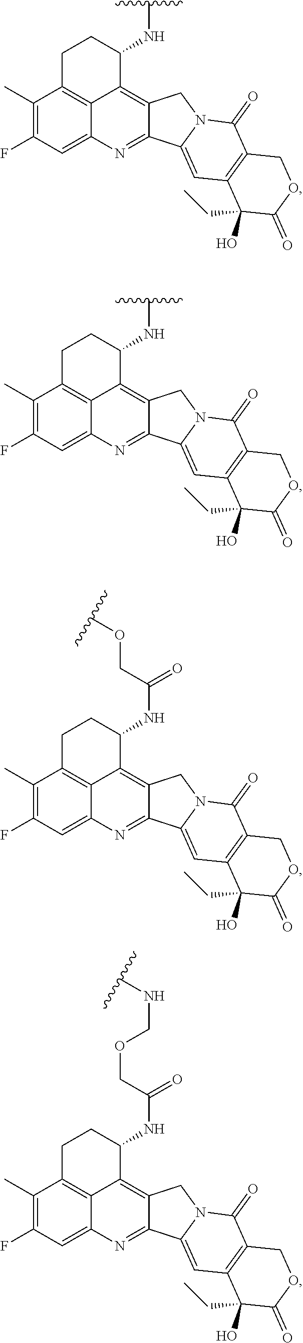 [SMILES: *N[C@H]1CCc2c(C)c(F)cc3nc4c(c1c23)Cn1c-4cc2c(c1=O)COC(=O)[C@]2(O)CC ; *N[C@H]1CCc2c(C)c(F)cc3nc4c(c1c23)Cn1c-4cc2c(c1=O)COC(=O)[C@]2(O)CC ; *OCC(=O)N[C@H]1CCc2c(C)c(F)cc3nc4c(c1c23)Cn1c-4cc2c(c1=O)COC(=O)[C@]2(O)CC ; *NCOCC(=O)N[C@H]1CCc2c(C)c(F)cc3nc4c(c1c23)Cn1c-4cc2c(c1=O)COC(=O)[C@]2(O)CC]

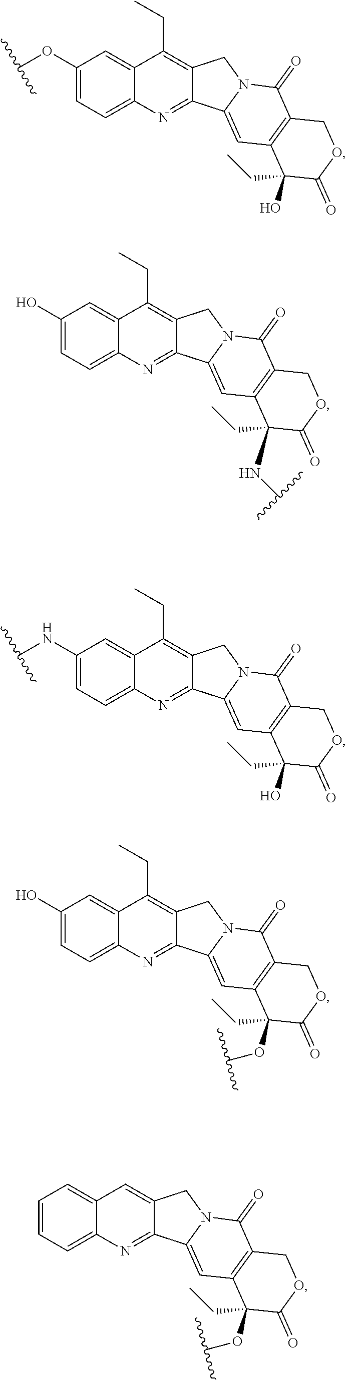 [SMILES: *Oc1ccc2nc3c(c(CC)c2c1)Cn1c-3cc2c(c1=O)COC(=O)[C@]2(O)CC ; *N[C@]1(CC)C(=O)OCc2c1cc1n(c2=O)Cc2c-1nc1ccc(O)cc1c2CC ; *Nc1ccc2nc3c(c(CC)c2c1)Cn1c-3cc2c(c1=O)COC(=O)[C@]2(O)CC ; *O[C@]1(CC)C(=O)OCc2c1cc1n(c2=O)Cc2c-1nc1ccc(O)cc1c2CC ; *O[C@]1(CC)C(=O)OCc2c1cc1n(c2=O)Cc2cc3ccccc3nc2-1]

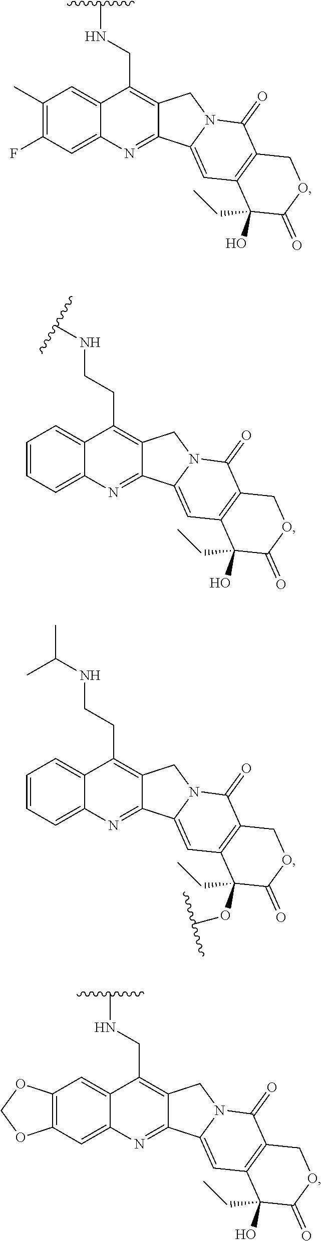 [SMILES: *NCc1c2c(nc3cc(F)c(C)cc13)-c1cc3c(c(=O)n1C2)COC(=O)[C@]3(O)CC ; *NCCc1c2c(nc3ccccc13)-c1cc3c(c(=O)n1C2)COC(=O)[C@]3(O)CC ; *O[C@]1(CC)C(=O)OCc2c1cc1n(c2=O)Cc2c-1nc1ccccc1c2CCNC(C)C ; *NCc1c2c(nc3cc4c(cc13)OCO4)-c1cc3c(c(=O)n1C2)COC(=O)[C@]3(O)CC]

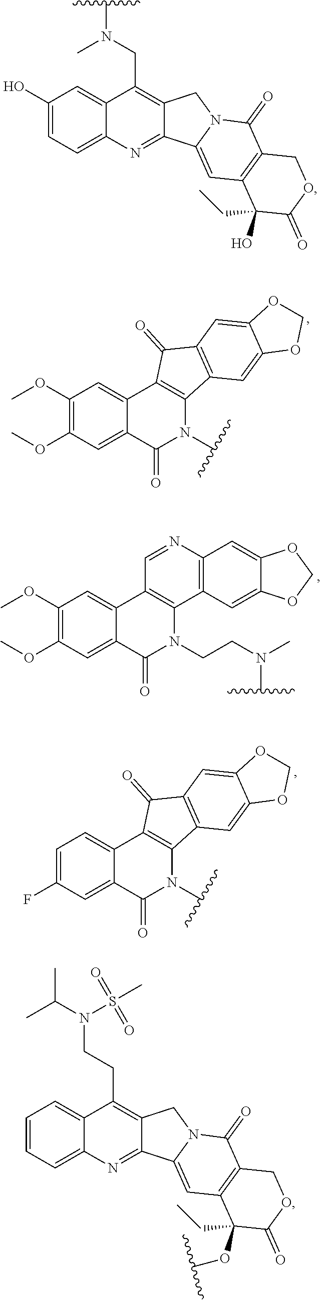 [SMILES: *N(C)Cc1c2c(nc3ccc(O)cc13)-c1cc3c(c(=O)n1C2)COC(=O)[C@]3(O)CC ; *n1c2c(c3cc(OC)c(OC)cc3c1=O)C(=O)c1cc3c(cc1-2)OCO3 ; *N(C)CCn1c(=O)c2cc(OC)c(OC)cc2c2cnc3cc4c(cc3c21)OCO4 ; *n1c2c(c3ccc(F)cc3c1=O)C(=O)c1cc3c(cc1-2)OCO3 ; *O[C@]1(CC)C(=O)OCc2c1cc1n(c2=O)Cc2c-1nc1ccccc1c2CCN(C(C)C)S(C)(=O)=O]

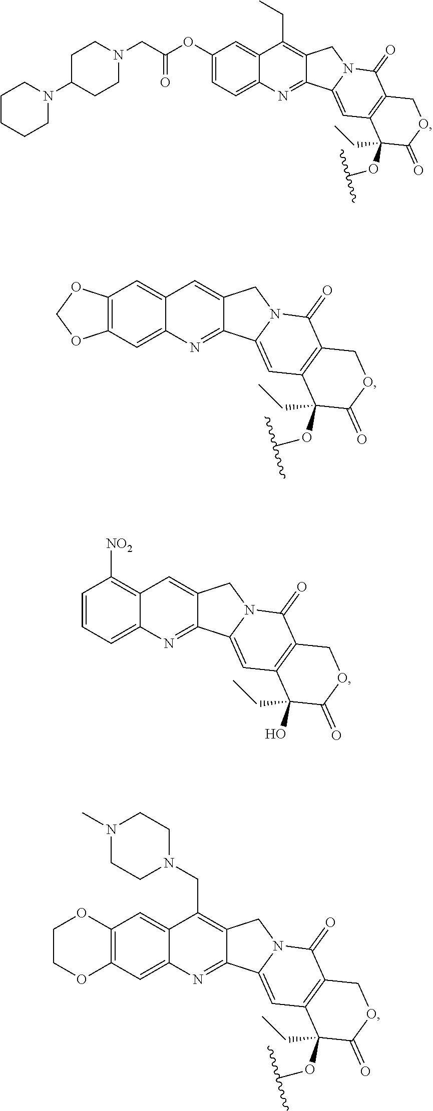 [SMILES: *O[C@]1(CC)C(=O)OCc2c1cc1n(c2=O)Cc2c-1nc1ccc(OC(=O)CN3CCC(N4CCCCC4)CC3)cc1c2CC ; *O[C@]1(CC)C(=O)OCc2c1cc1n(c2=O)Cc2cc3cc4c(cc3nc2-1)OCO4 ; CC[C@@]1(O)C(=O)OCc2c1cc1n(c2=O)Cc2cc3c([N+](=O)[O-])cccc3nc2-1 ; *O[C@]1(CC)C(=O)OCc2c1cc1n(c2=O)Cc2c-1nc1cc3c(cc1c2CN1CCN(C)CC1)OCCO3]

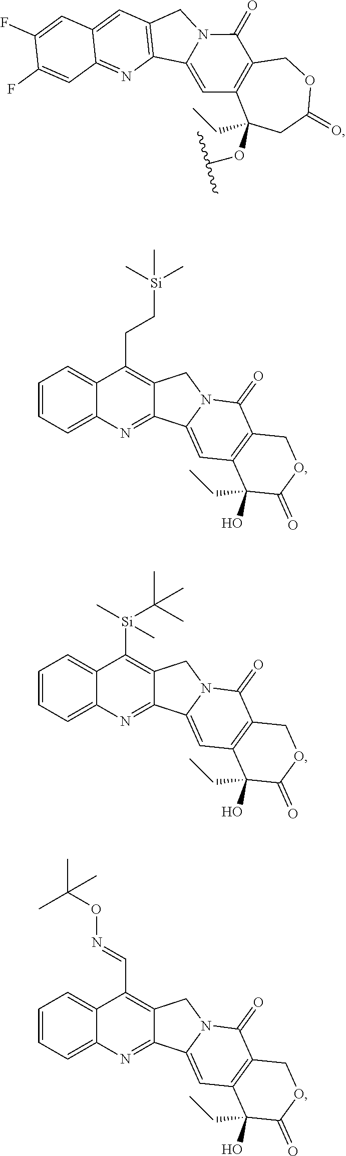 [SMILES: *O[C@]1(CC)CC(=O)OCc2c1cc1n(c2=O)Cc2cc3cc(F)c(F)cc3nc2-1 ; CC[C@@]1(O)C(=O)OCc2c1cc1n(c2=O)Cc2c-1nc1ccccc1c2CC[Si](C)(C)C ; CC[C@@]1(O)C(=O)OCc2c1cc1n(c2=O)Cc2c-1nc1ccccc1c2[Si](C)(C)C(C)(C)C ; CC[C@@]1(O)C(=O)OCc2c1cc1n(c2=O)Cc2c-1nc1ccccc1c2/C=N/OC(C)(C)C]

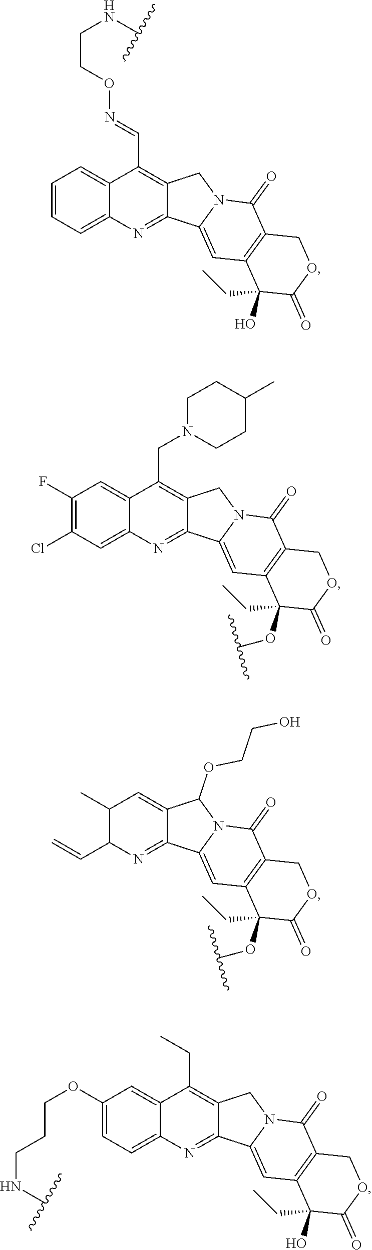 [SMILES: *NCCO/N=C/c1c2c(nc3ccccc13)-c1cc3c(c(=O)n1C2)COC(=O)[C@]3(O)CC ; *O[C@]1(CC)C(=O)OCc2c1cc1n(c2=O)Cc2c-1nc1cc(Cl)c(F)cc1c2CN1CCC(C)CC1 ; *O[C@]1(CC)C(=O)OCc2c1cc1n(c2=O)C(OCCO)C2=CC(C)C(C=C)N=C21 ; *NCCCOc1ccc2nc3c(c(CC)c2c1)Cn1c-3cc2c(c1=O)COC(=O)[C@]2(O)CC]

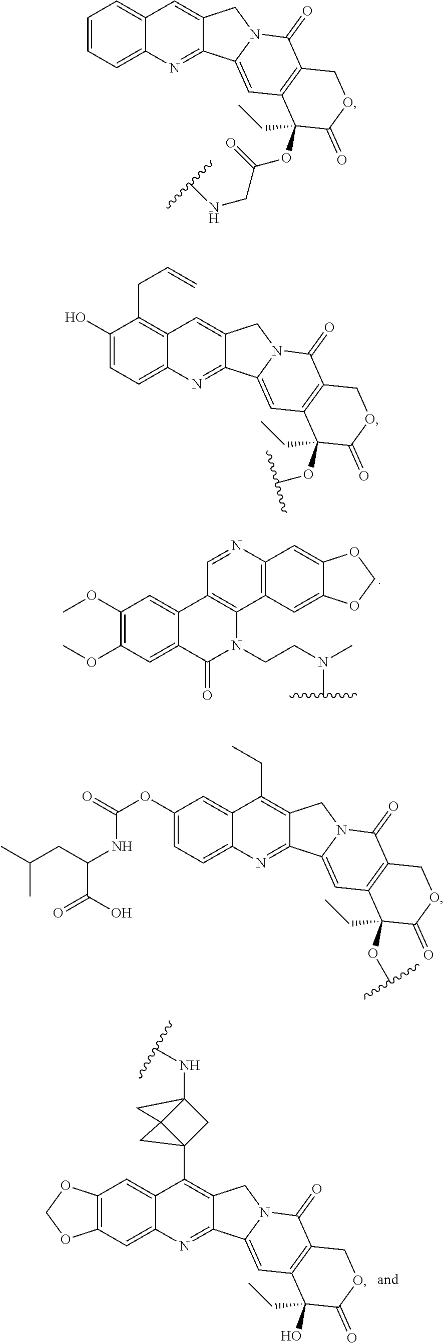 [SMILES: *NCC(=O)O[C@]1(CC)C(=O)OCc2c1cc1n(c2=O)Cc2cc3ccccc3nc2-1 ; *O[C@]1(CC)C(=O)OCc2c1cc1n(c2=O)Cc2cc3c(CC=C)c(O)ccc3nc2-1 ; *N(C)CCn1c(=O)c2cc(OC)c(OC)cc2c2cnc3cc4c(cc3c21)OCO4 ; *O[C@]1(CC)C(=O)OCc2c1cc1n(c2=O)Cc2c-1nc1ccc(OC(=O)NC(CC(C)C)C(=O)O)cc1c2CC ; *NC12CC3(c4c5c(nc6cc7c(cc46)OCO7)-c4cc6c(c(=O)n4C5)COC(=O)[C@]6(O)CC)CC13C2]

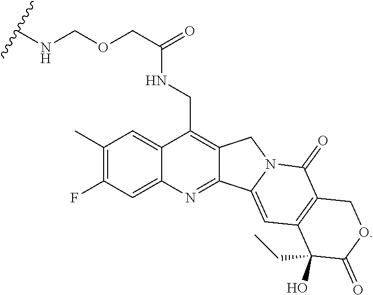 [SMILES: *NCOCC(=O)NCc1c2c(nc3cc(F)c(C)cc13)-c1cc3c(c(=O)n1C2)COC(=O)[C@]3(O)CC]

Embodiment 4. The conjugate of any one of embodiments 1-3, wherein the linker L comprises or consists of the following formula:

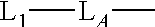

wherein L_{A} is a linker and L₁ is a linking moiety.

Embodiment 5. The conjugate of embodiment 3, wherein the linker L_{A} comprises or consists of the formula:

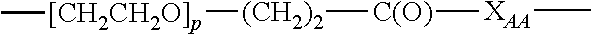

wherein X_{AA} is an amino acid sequence, and p is an integer from 0 to 50, optionally wherein linker L_{A} further comprises

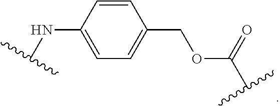 [SMILES: *Nc1ccc(COC(*)=O)cc1]

Embodiment 6. The conjugate of embodiment 5, wherein p is 4 or p is 8 or p is 0.

Embodiment 7. The conjugate of embodiment 5 or embodiment 6, wherein X_{AA} is valine-alanine.

Embodiment 8. The conjugate of any one of embodiments 3-7, wherein L₁ is or comprises

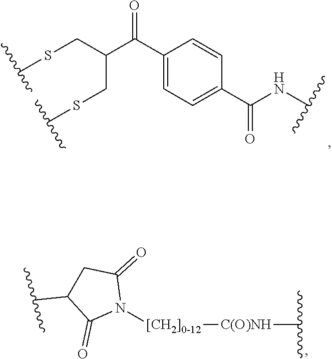

optionally

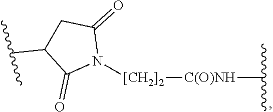

optionally

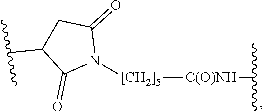

optionally further comprising

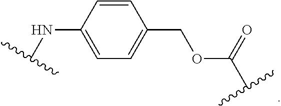 [SMILES: *Nc1ccc(COC(*)=O)cc1]

Embodiment 9. The conjugate of embodiment 1, wherein the linker L comprises or consists of:

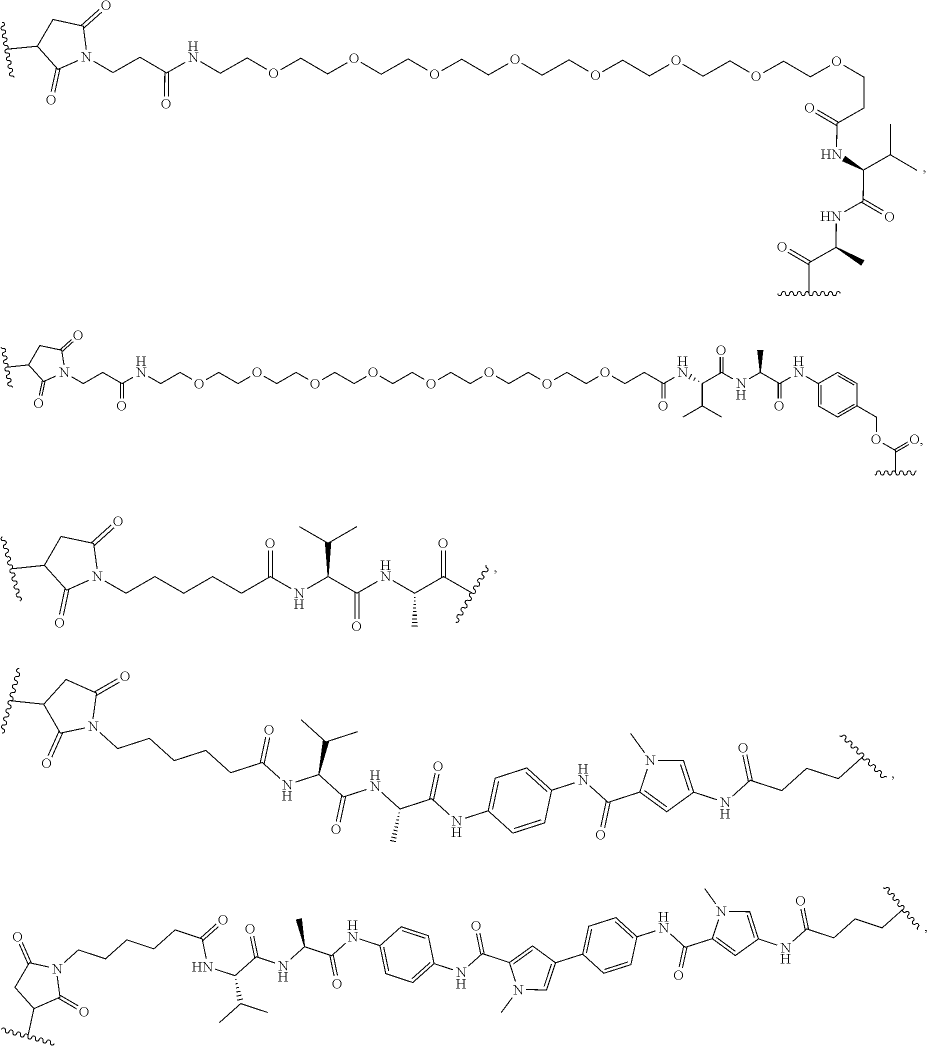 [SMILES: *C(=O)[C@H](C)NC(=O)[C@@H](NC(=O)CCOCCOCCOCCOCCOCCOCCOCCOCCNC(=O)CCN1C(=O)CC(*)C1=O)C(C)C ; *C(=O)OCc1ccc(NC(=O)[C@H](C)NC(=O)[C@@H](NC(=O)CCOCCOCCOCCOCCOCCOCCOCCOCCNC(=O)CCN2C(=O)CC(*)C2=O)C(C)C)cc1 ; *C(=O)[C@H](C)NC(=O)[C@@H](NC(=O)CCCCCN1C(=O)CC(*)C1=O)C(C)C ; *CCCC(=O)Nc1cc(C(=O)Nc2ccc(NC(=O)[C@H](C)NC(=O)[C@@H](NC(=O)CCCCCN3C(=O)CC(*)C3=O)C(C)C)cc2)n(C)c1 ; *CCCC(=O)Nc1cc(C(=O)Nc2ccc(-c3cc(C(=O)Nc4ccc(NC(=O)[C@H](C)NC(=O)[C@@H](NC(=O)CCCCCN5C(=O)CC(*)C5=O)C(C)C)cc4)n(C)c3)cc2)n(C)c1]

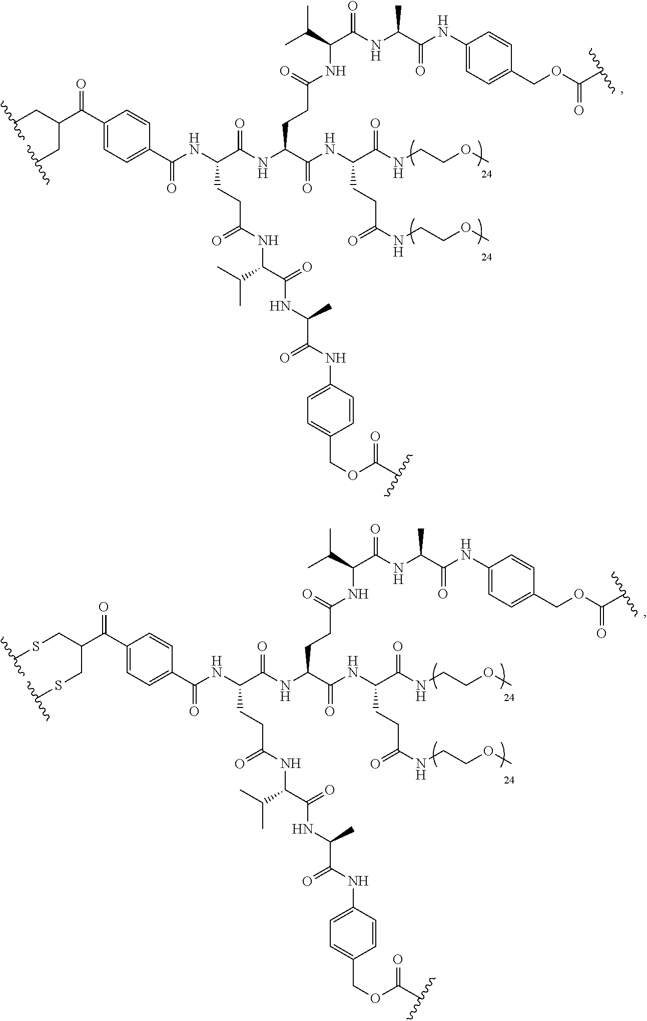

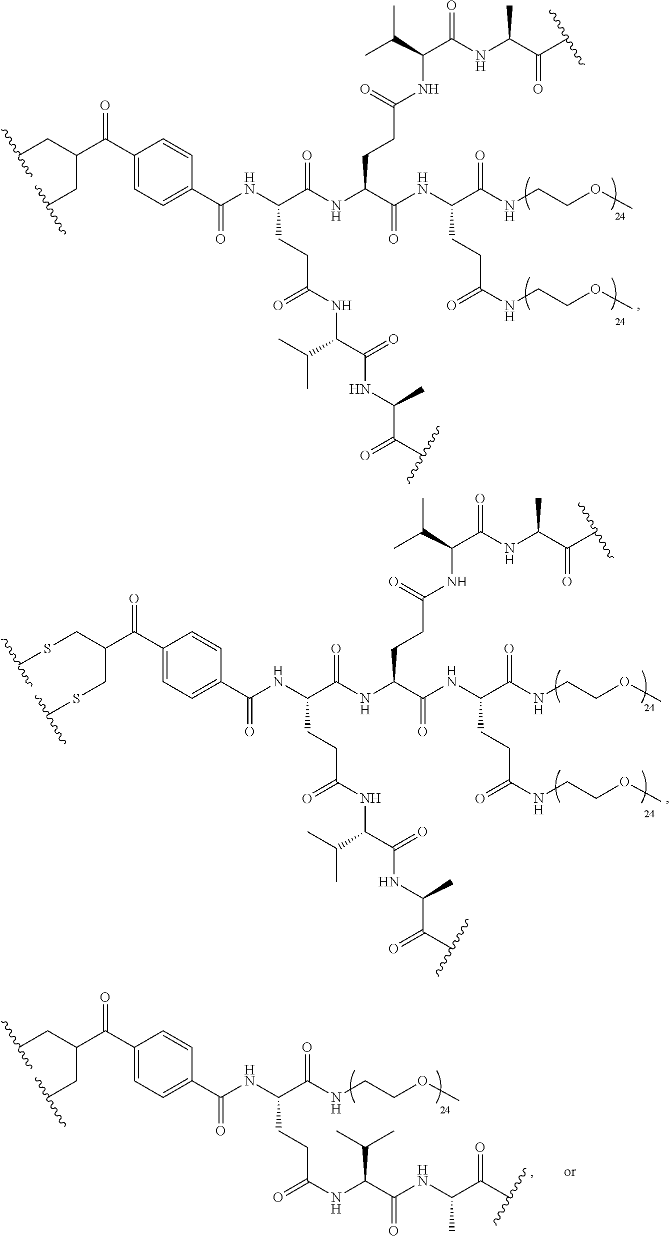

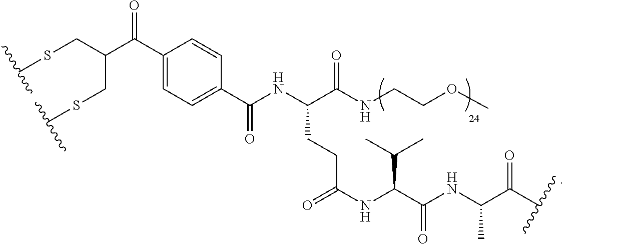

Embodiment 10. The conjugate of any one of embodiments 1-9, wherein the antibody or binding fragment thereof specifically binds CUB Domain-Containing Protein-1 (CDCP1).

Embodiment 11. The conjugate of embodiment 10, wherein the antibody or binding fragment thereof comprises:
(i) a heavy chain variable region (VH) that comprises:
   (a) a VH complementarity determining region one (CDRH1) comprising and/or consisting of the amino acid sequence of SEQ ID NO: 2,
   (b) a VH complementarity determining region two (CDRH2) comprising and/or consisting of the amino acid sequence of SEQ ID NO: 3, and
   (c) a VH complementarity determining region three (CDRH3) comprising and/or consisting of the amino acid sequence of SEQ ID NO: 4,
and (ii) a light chain variable region (VL) that comprises:
   (a) a VL complementarity determining region one (CDRL1) comprising and/or consisting of the amino acid sequence of SEQ ID NO: 6,
   (b) a VL complementarity determining region two (CDRL2) comprising and/or consisting of the amino acid sequence of SEQ ID NO: 7, and
   (c) a VL complementarity determining region three (CDRL3) comprising and/or consisting of the amino acid sequence of SEQ ID NO:8.

Embodiment 12. The conjugate of embodiment 10, wherein the antibody or binding fragment thereof comprises:
(i) a heavy chain variable region (VH) that comprises:
   (a) a VH complementarity determining region one (CDRH1) comprising and/or consisting of the amino acid sequence of SEQ ID NO: 12,
   (b) a VH complementarity determining region two (CDRH2) comprising and/or consisting of the amino acid sequence of SEQ ID NO: 13, and
   (c) a VH complementarity determining region three (CDRH3) comprising and/or consisting of the amino acid sequence of SEQ ID NO: 14,
and (ii) a light chain variable region (VL) that comprises:
   (a) a VL complementarity determining region one (CDRL1) comprising and/or consisting of the amino acid sequence of SEQ ID NO: 6,
   (b) a VL complementarity determining region two (CDRL2) comprising and/or consisting of the amino acid sequence of SEQ ID NO: 7, and
   (c) a VL complementarity determining region three (CDRL3) comprising and/or consisting of the amino acid sequence of SEQ ID NO:8.

Embodiment 13. The conjugate of any one of embodiments 1-12, wherein the antibody or binding fragment thereof comprises a VH that comprises an amino acid sequence at least 90%, at least 91%, at least 92%, at least 93%, at least 94%, at least 95%, at least 96%, at least 97%, at least 98%, at least 99%, or 100% identical to the amino acid sequence of SEQ ID NO: 1, and a VL that comprises an amino acid sequence at least 90%, at least 91%, at least 92%, at least 93%, at least 94%, at least 95%, at least 96%, at least 97%, at least 98%, at least 99%, or 100% identical to the amino acid sequence of SEQ ID NO: 5.

Embodiment 14. The conjugate of embodiment 13, wherein the antibody or binding fragment thereof comprises a VH that comprises the amino acid sequence of SEQ ID NO: 1 and a VL that comprises the amino acid sequence of SEQ ID NO: 5.

Embodiment 15. The conjugate of any one of embodiments 1-14, wherein the antibody or binding fragment thereof comprises a heavy chain comprising an amino acid sequence at least 90%, at least 91%, at least 92%, at least 93%, at least 94%, at least 95%, at least 96%, at least 97%, at least 98%, at least 99%, or 100% identical to the amino acid sequence of SEQ ID NO: 1 and a light chain comprising an amino acid sequence at least 90%, at least 91%, at least 92%, at least 93%, at least 94%, at least 95%, at least 96%, at least 97%, at least 98%, at least 99%, or 100% identical to the amino acid sequence of SEQ ID NO: 5.

Embodiment 16. The conjugate of embodiment 15, wherein the antibody or binding fragment thereof comprises a heavy chain that comprises the amino acid sequence of SEQ ID NO: 1 and a light chain that comprises the amino acid sequence of SEQ ID NO: 5.

Embodiment 17. An antibody-drug conjugate having formula (I):

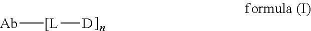

wherein in formula (I):
Ab comprises an antibody or antibody-binding fragment, wherein the antibody or binding fragment thereof specifically binds CUB Domain-Containing Protein-1 (CDCP1);
L is a linker;
D comprises a drug moiety; and
n is an integer from 1 to 20.

Embodiment 18. The antibody-drug conjugate of embodiment 16, wherein the drug moiety D is selected from exatecan, Dxd, Sn-38, monomethyl auristatin E (MMAE), and a pyrridinobenzodiazepine (PDD).

Embodiment 19. The antibody-drug conjugate of embodiment 17 or 18, wherein the exatecan comprises or has the formula:

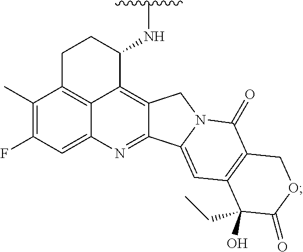 [SMILES: *N[C@H]1CCc2c(C)c(F)cc3nc4c(c1c23)Cn1c-4cc2c(c1=O)COC(=O)[C@]2(O)CC]

or wherein the PDD comprises or has the formula:

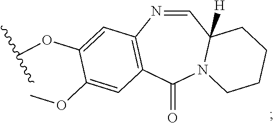 [SMILES: *Oc1cc2c(cc1OC)C(=O)N1CCCC[C@H]1C=N2]

or wherein the PDD comprises or has the formula:

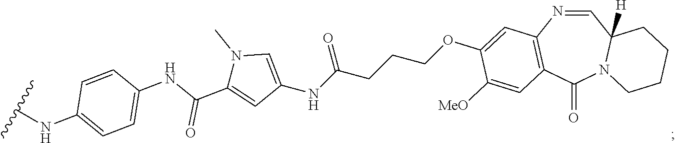 [SMILES: *Nc1ccc(NC(=O)c2cc(NC(=O)CCCOc3cc4c(cc3OC)C(=O)N3CCCC[C@H]3C=N4)cn2C)cc1]

or wherein the PDD comprises or has the formula:

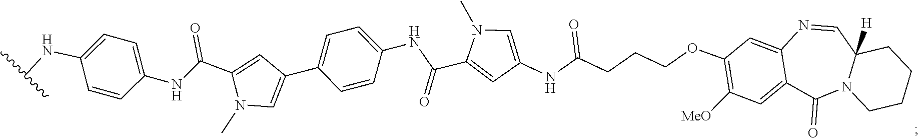 [SMILES: *Nc1ccc(NC(=O)c2cc(-c3ccc(NC(=O)c4cc(NC(=O)CCCOc5cc6c(cc5OC)C(=O)N5CCCC[C@H]5C=N6)cn4C)cc3)cn2C)cc1]

or wherein the Sn-38 comprises or has the formula:

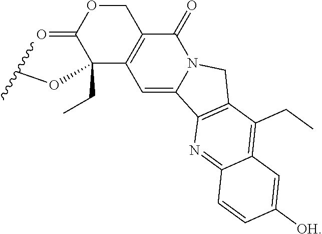 [SMILES: *O[C@]1(CC)C(=O)OCc2c1cc1n(c2=O)Cc2c-1nc1ccc(O)cc1c2CC]

Embodiment 19a: The conjugate of any one of embodiments 17-19, wherein the drug moiety D is selected from.

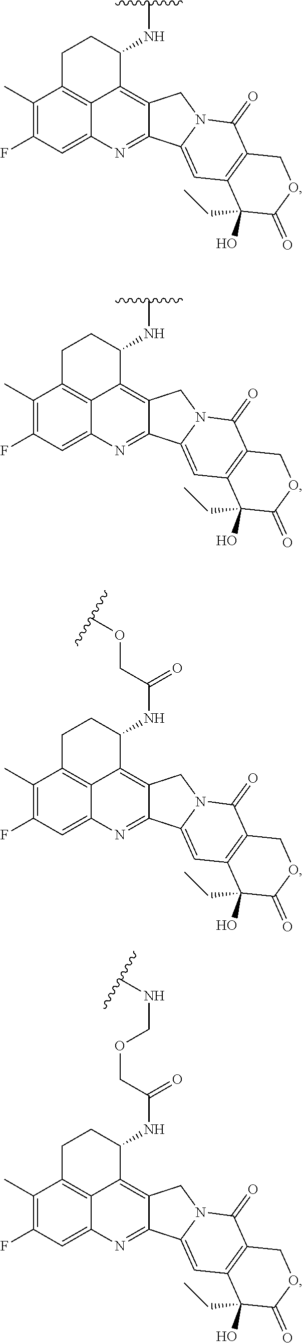 [SMILES: *N[C@H]1CCc2c(C)c(F)cc3nc4c(c1c23)Cn1c-4cc2c(c1=O)COC(=O)[C@]2(O)CC ; *N[C@H]1CCc2c(C)c(F)cc3nc4c(c1c23)Cn1c-4cc2c(c1=O)COC(=O)[C@]2(O)CC ; *OCC(=O)N[C@H]1CCc2c(C)c(F)cc3nc4c(c1c23)Cn1c-4cc2c(c1=O)COC(=O)[C@]2(O)CC ; *NCOCC(=O)N[C@H]1CCc2c(C)c(F)cc3nc4c(c1c23)Cn1c-4cc2c(c1=O)COC(=O)[C@]2(O)CC]

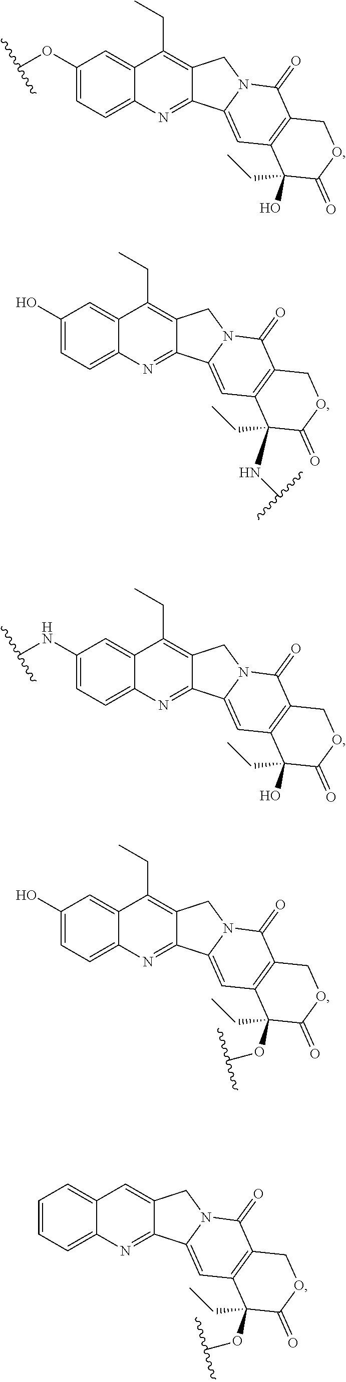 [SMILES: *Oc1ccc2nc3c(c(CC)c2c1)Cn1c-3cc2c(c1=O)COC(=O)[C@]2(O)CC ; *N[C@]1(CC)C(=O)OCc2c1cc1n(c2=O)Cc2c-1nc1ccc(O)cc1c2CC ; *Nc1ccc2nc3c(c(CC)c2c1)Cn1c-3cc2c(c1=O)COC(=O)[C@]2(O)CC ; *O[C@]1(CC)C(=O)OCc2c1cc1n(c2=O)Cc2c-1nc1ccc(O)cc1c2CC ; *O[C@]1(CC)C(=O)OCc2c1cc1n(c2=O)Cc2cc3ccccc3nc2-1]

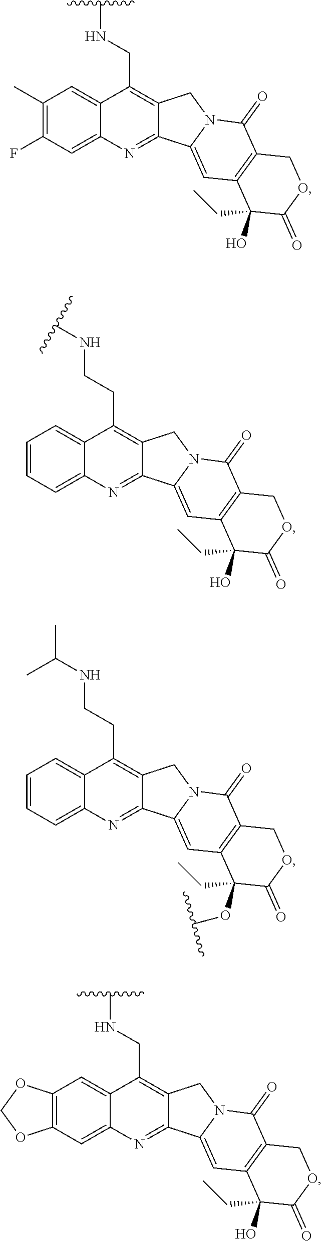 [SMILES: *NCc1c2c(nc3cc(F)c(C)cc13)-c1cc3c(c(=O)n1C2)COC(=O)[C@]3(O)CC ; *NCCc1c2c(nc3ccccc13)-c1cc3c(c(=O)n1C2)COC(=O)[C@]3(O)CC ; *O[C@]1(CC)C(=O)OCc2c1cc1n(c2=O)Cc2c-1nc1ccccc1c2CCNC(C)C ; *NCc1c2c(nc3cc4c(cc13)OCO4)-c1cc3c(c(=O)n1C2)COC(=O)[C@]3(O)CC]

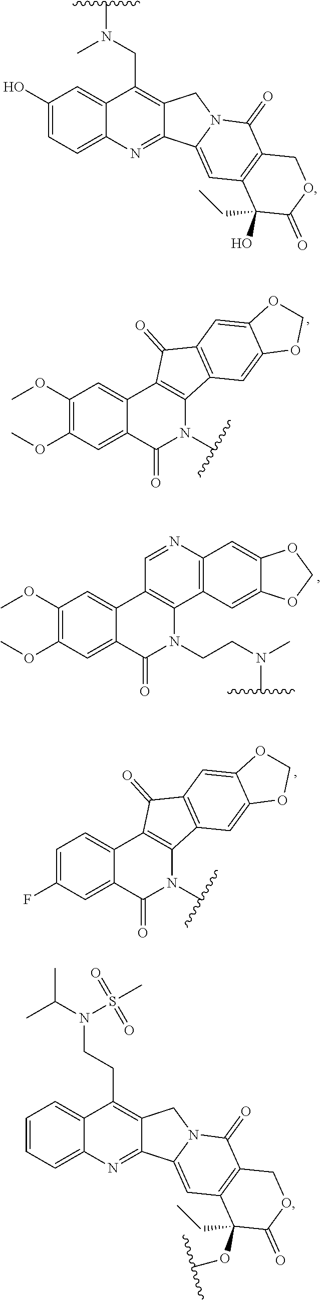 [SMILES: *N(C)Cc1c2c(nc3ccc(O)cc13)-c1cc3c(c(=O)n1C2)COC(=O)[C@]3(O)CC ; *n1c2c(c3cc(OC)c(OC)cc3c1=O)C(=O)c1cc3c(cc1-2)OCO3 ; *N(C)CCn1c(=O)c2cc(OC)c(OC)cc2c2cnc3cc4c(cc3c21)OCO4 ; *n1c2c(c3ccc(F)cc3c1=O)C(=O)c1cc3c(cc1-2)OCO3 ; *O[C@]1(CC)C(=O)OCc2c1cc1n(c2=O)Cc2c-1nc1ccccc1c2CCN(C(C)C)S(C)(=O)=O]

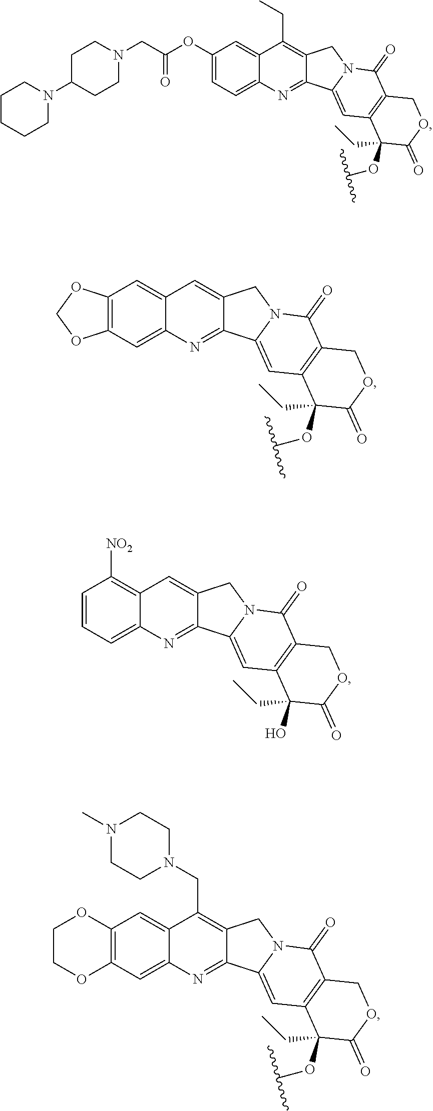 [SMILES: *O[C@]1(CC)C(=O)OCc2c1cc1n(c2=O)Cc2c-1nc1ccc(OC(=O)CN3CCC(N4CCCCC4)CC3)cc1c2CC ; *O[C@]1(CC)C(=O)OCc2c1cc1n(c2=O)Cc2cc3cc4c(cc3nc2-1)OCO4 ; CC[C@@]1(O)C(=O)OCc2c1cc1n(c2=O)Cc2cc3c([N+](=O)[O-])cccc3nc2-1 ; *O[C@]1(CC)C(=O)OCc2c1cc1n(c2=O)Cc2c-1nc1cc3c(cc1c2CN1CCN(C)CC1)OCCO3]

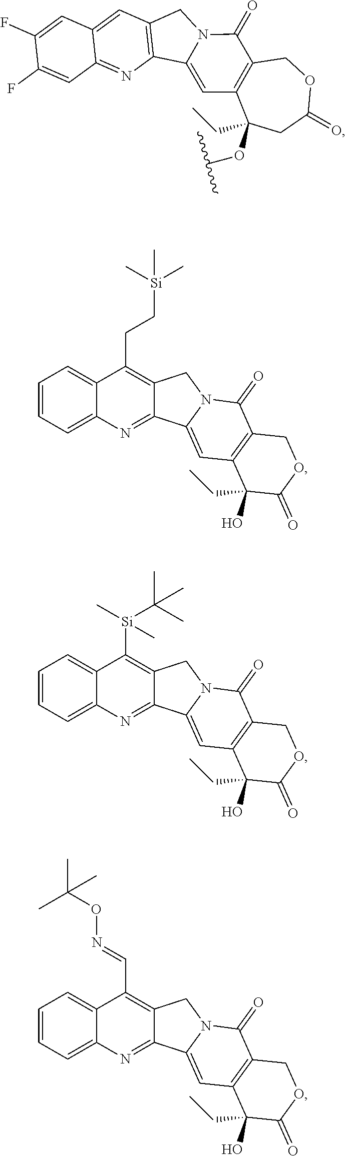 [SMILES: *O[C@]1(CC)CC(=O)OCc2c1cc1n(c2=O)Cc2cc3cc(F)c(F)cc3nc2-1 ; CC[C@@]1(O)C(=O)OCc2c1cc1n(c2=O)Cc2c-1nc1ccccc1c2CC[Si](C)(C)C ; CC[C@@]1(O)C(=O)OCc2c1cc1n(c2=O)Cc2c-1nc1ccccc1c2[Si](C)(C)C(C)(C)C ; CC[C@@]1(O)C(=O)OCc2c1cc1n(c2=O)Cc2c-1nc1ccccc1c2/C=N/OC(C)(C)C]

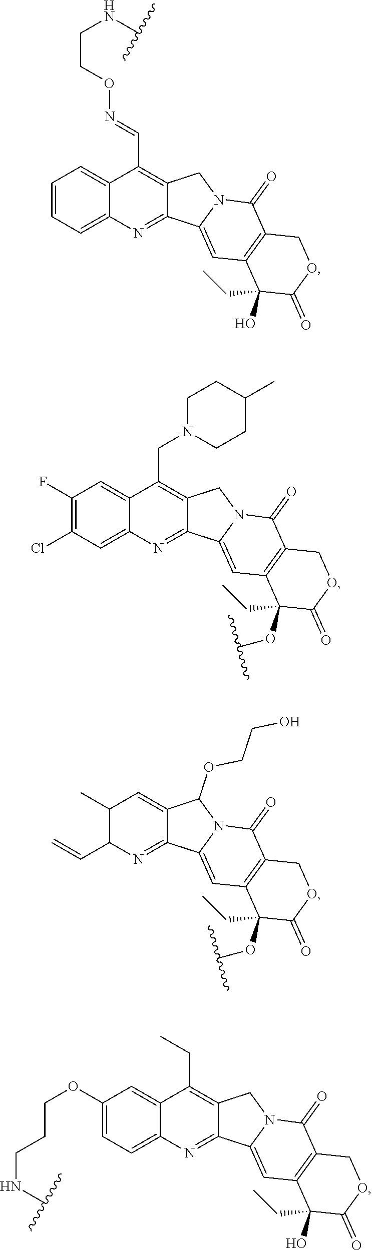 [SMILES: *NCCO/N=C/c1c2c(nc3ccccc13)-c1cc3c(c(=O)n1C2)COC(=O)[C@]3(O)CC ; *O[C@]1(CC)C(=O)OCc2c1cc1n(c2=O)Cc2c-1nc1cc(Cl)c(F)cc1c2CN1CCC(C)CC1 ; *O[C@]1(CC)C(=O)OCc2c1cc1n(c2=O)C(OCCO)C2=CC(C)C(C=C)N=C21 ; *NCCCOc1ccc2nc3c(c(CC)c2c1)Cn1c-3cc2c(c1=O)COC(=O)[C@]2(O)CC]

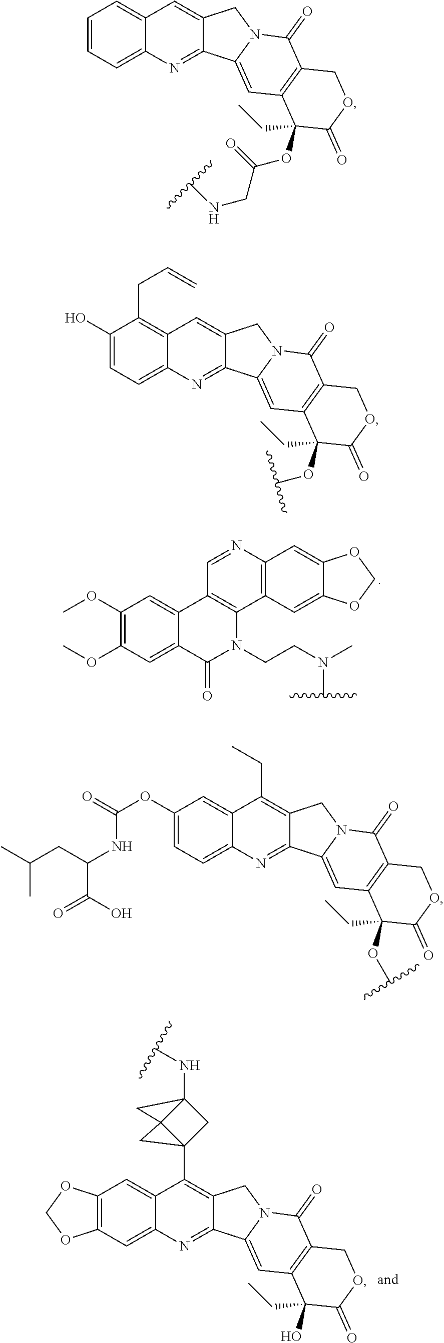 [SMILES: *NCC(=O)O[C@]1(CC)C(=O)OCc2c1cc1n(c2=O)Cc2cc3ccccc3nc2-1 ; *O[C@]1(CC)C(=O)OCc2c1cc1n(c2=O)Cc2cc3c(CC=C)c(O)ccc3nc2-1 ; *N(C)CCn1c(=O)c2cc(OC)c(OC)cc2c2cnc3cc4c(cc3c21)OCO4 ; *O[C@]1(CC)C(=O)OCc2c1cc1n(c2=O)Cc2c-1nc1ccc(OC(=O)NC(CC(C)C)C(=O)O)cc1c2CC ; *NC12CC3(c4c5c(nc6cc7c(cc46)OCO7)-c4cc6c(c(=O)n4C5)COC(=O)[C@]6(O)CC)CC13C2]

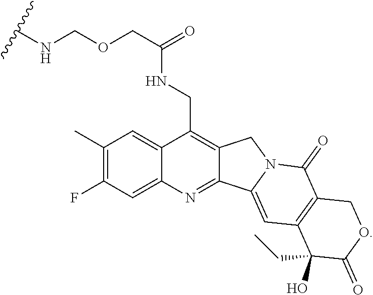 [SMILES: *NCOCC(=O)NCc1c2c(nc3cc(F)c(C)cc13)-c1cc3c(c(=O)n1C2)COC(=O)[C@]3(O)CC]

Embodiment 20. The antibody-drug conjugate of any one of embodiments 17-19, wherein the linker L comprises or consists of the following formula:

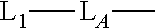

wherein L_{A} is a linker and L₁ is a linking moiety.

Embodiment 21. The antibody-drug conjugate of embodiment 20, wherein the linker L_{A} comprises or consists of the formula:

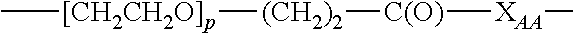

wherein X_{AA} is an amino acid sequence, and p is an integer from 0 to 50, optionally wherein linker L_{A} further comprises

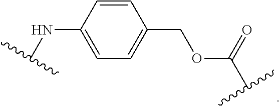 [SMILES: *Nc1ccc(COC(*)=O)cc1]

Embodiment 22. The antibody-drug conjugate of embodiment 21, wherein p is 8 or p is 0.

Embodiment 23. The antibody-drug conjugate of embodiment 21 or 22, wherein X_{AA} is valine-alanine.

Embodiment 24. The antibody-drug conjugate of any one of embodiments 21-23, wherein L₁ is or comprises

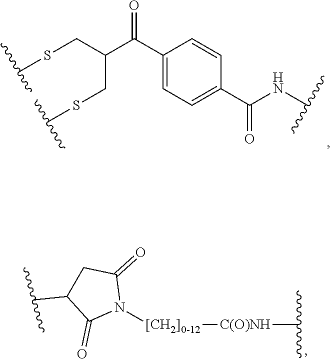

optionally

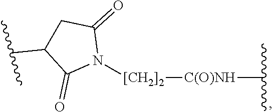

optionally

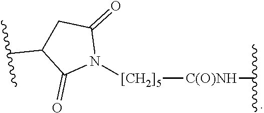

Embodiment 25. The antibody-drug conjugate of embodiment 24, wherein L₁ further comprises

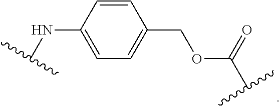 [SMILES: *Nc1ccc(COC(*)=O)cc1]

Embodiment 26. The antibody-drug conjugate of any one of embodiments 17-25, wherein the linker L comprises or consists of:

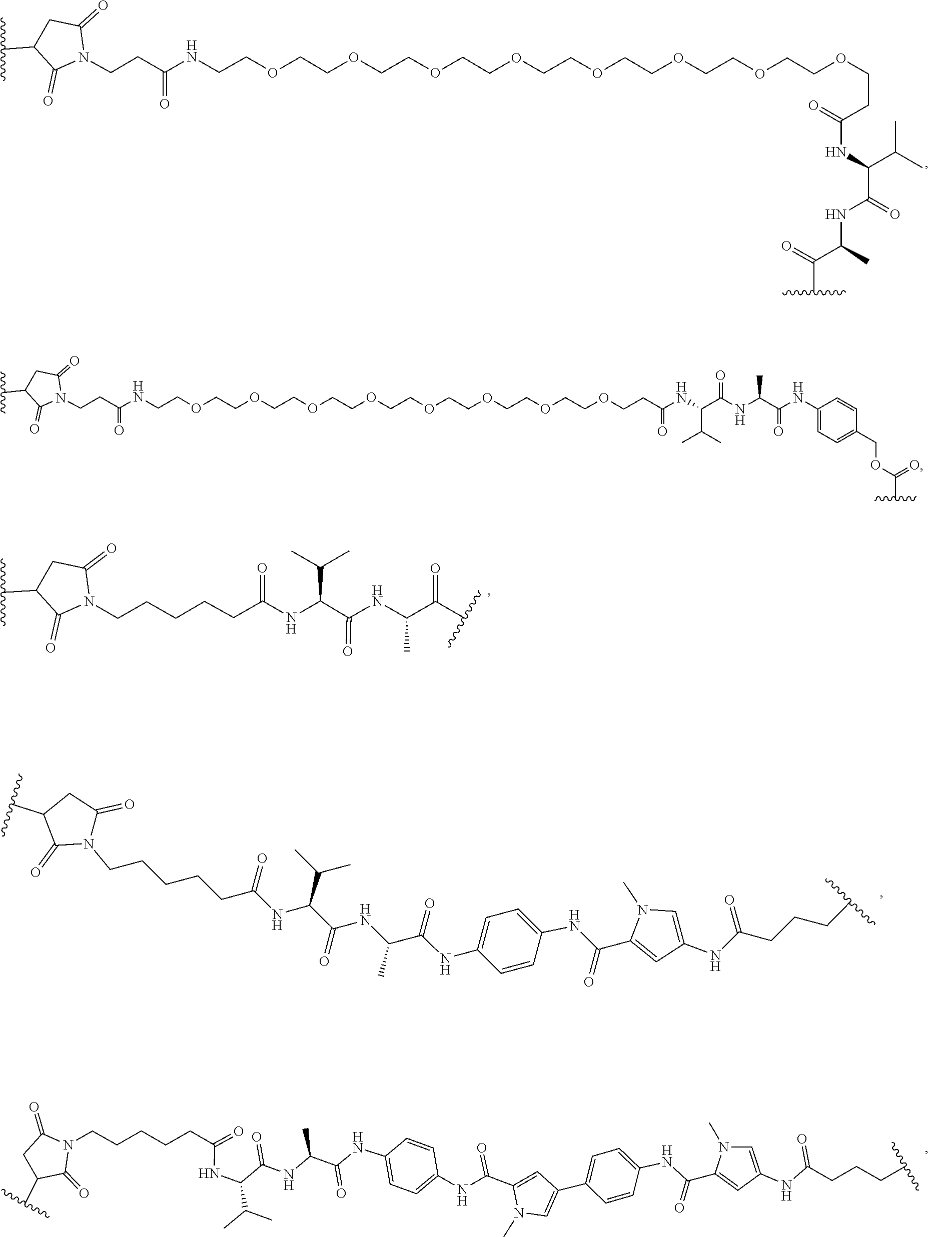 [SMILES: *C(=O)[C@H](C)NC(=O)[C@@H](NC(=O)CCOCCOCCOCCOCCOCCOCCOCCOCCNC(=O)CCN1C(=O)CC(*)C1=O)C(C)C ; *C(=O)OCc1ccc(NC(=O)[C@H](C)NC(=O)[C@@H](NC(=O)CCOCCOCCOCCOCCOCCOCCOCCOCCNC(=O)CCN2C(=O)CC(*)C2=O)C(C)C)cc1 ; *C(=O)[C@H](C)NC(=O)[C@@H](NC(=O)CCCCCN1C(=O)CC(*)C1=O)C(C)C ; *CCCC(=O)Nc1cc(C(=O)Nc2ccc(NC(=O)[C@H](C)NC(=O)[C@@H](NC(=O)CCCCCN3C(=O)CC(*)C3=O)C(C)C)cc2)n(C)c1 ; *CCCC(=O)Nc1cc(C(=O)Nc2ccc(-c3cc(C(=O)Nc4ccc(NC(=O)[C@H](C)NC(=O)[C@@H](NC(=O)CCCCCN5C(=O)CC(*)C5=O)C(C)C)cc4)n(C)c3)cc2)n(C)c1]

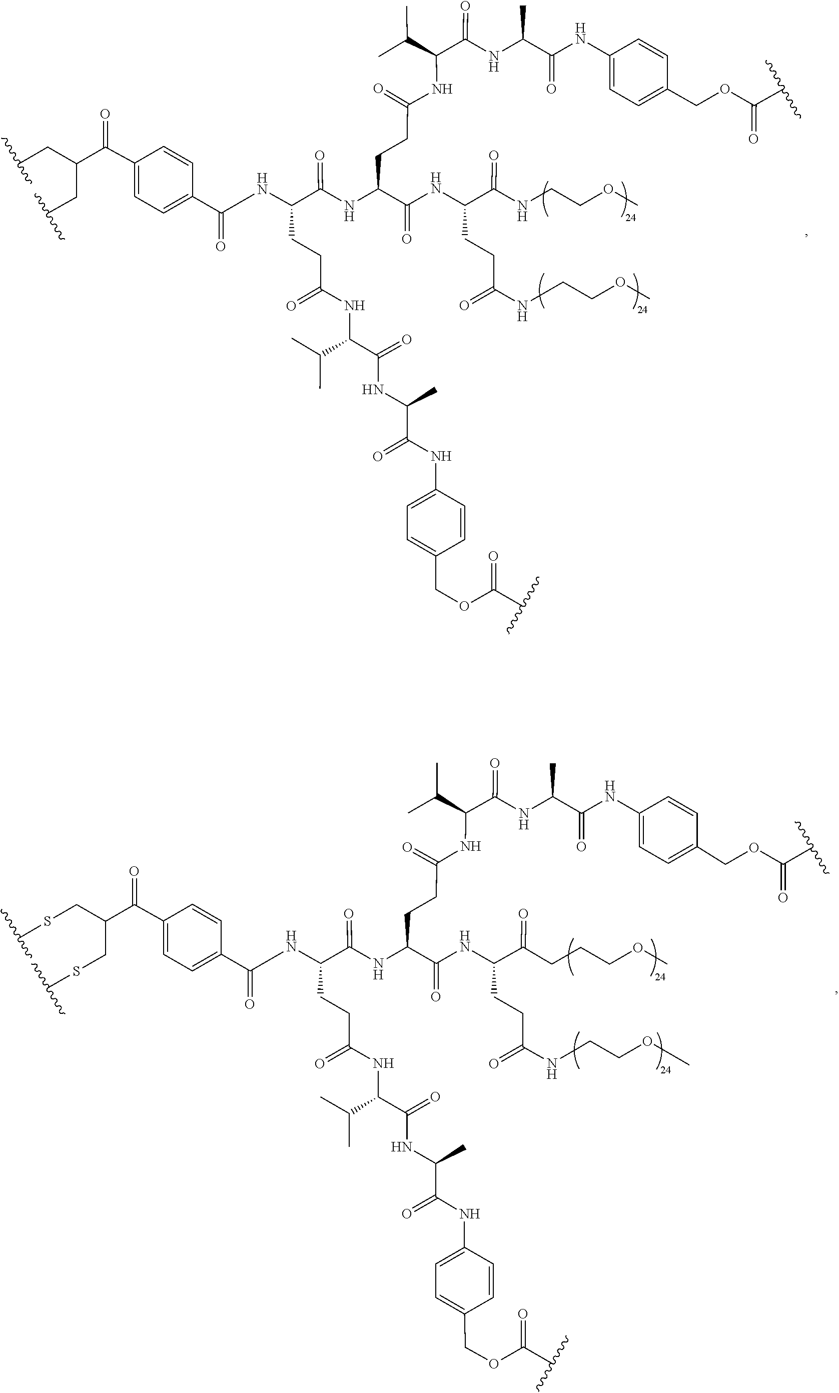

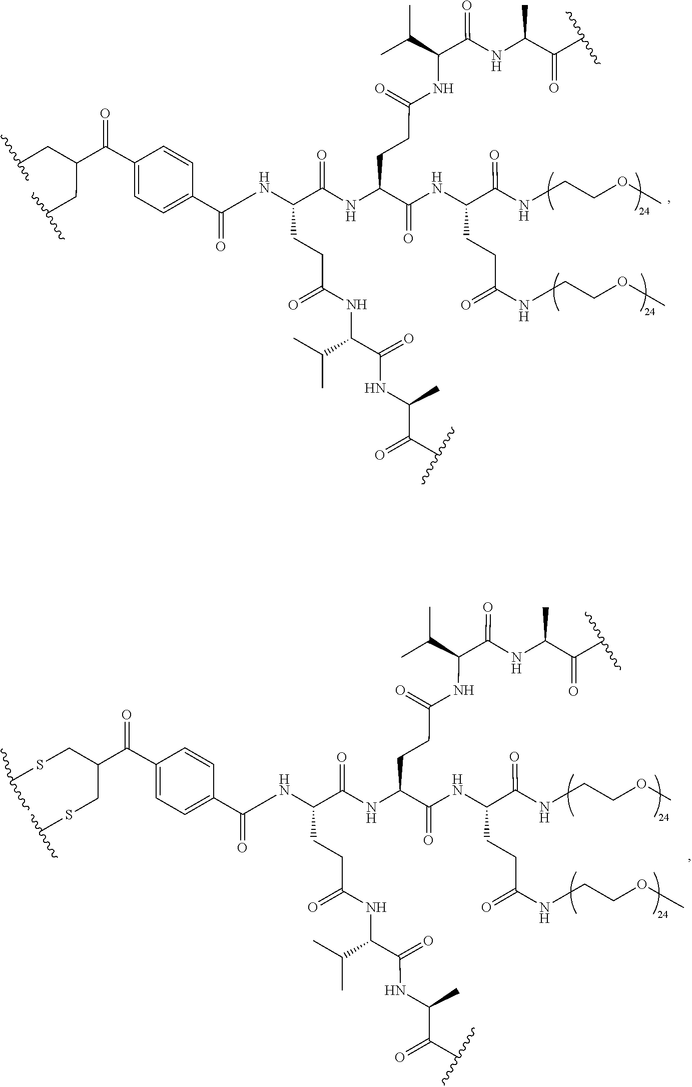

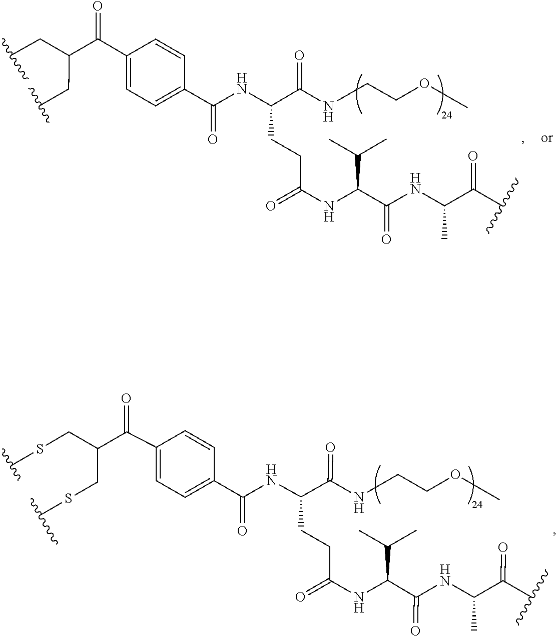

Embodiment 27. The antibody-drug conjugate of embodiment 17, wherein L-D has the formula:

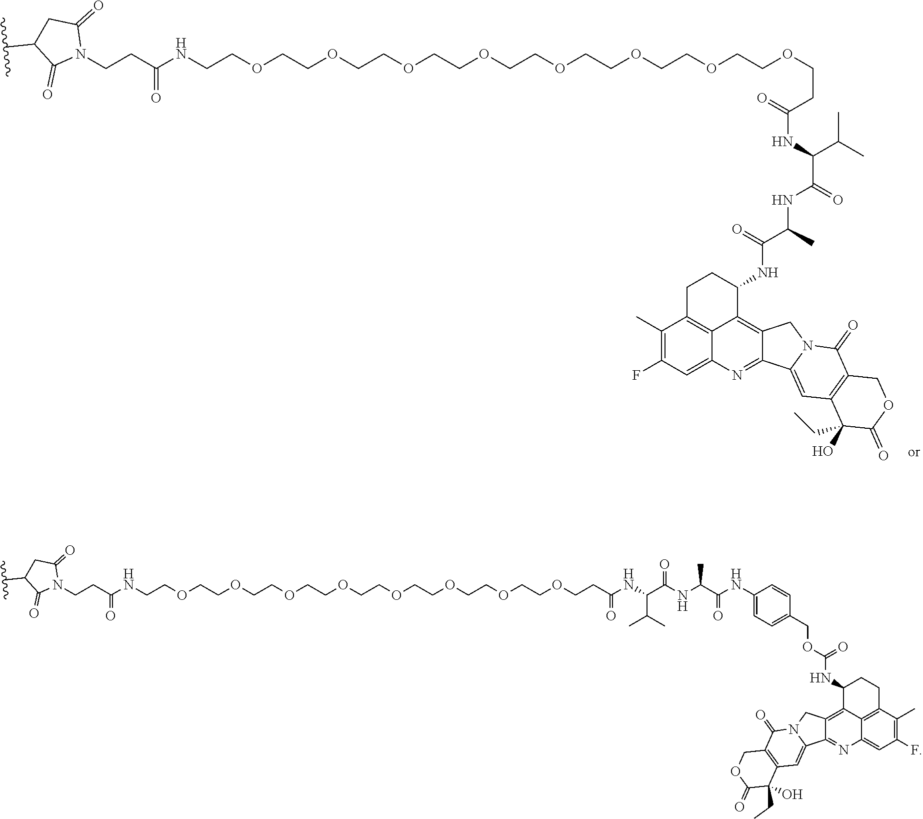 [SMILES: *C1CC(=O)N(CCC(=O)NCCOCCOCCOCCOCCOCCOCCOCCOCCC(=O)N[C@H](C(=O)N[C@@H](C)C(=O)N[C@H]2CCc3c(C)c(F)cc4nc5c(c2c34)Cn2c-5cc3c(c2=O)COC(=O)[C@]3(O)CC)C(C)C)C1=O ; *C1CC(=O)N(CCC(=O)NCCOCCOCCOCCOCCOCCOCCOCCOCCC(=O)N[C@H](C(=O)N[C@@H](C)C(=O)Nc2ccc(COC(=O)N[C@H]3CCc4c(C)c(F)cc5nc6c(c3c45)Cn3c-6cc4c(c3=O)COC(=O)[C@]4(O)CC)cc2)C(C)C)C1=O]

Embodiment 28. The antibody-drug conjugate of embodiment 17, wherein L-D has the formula:

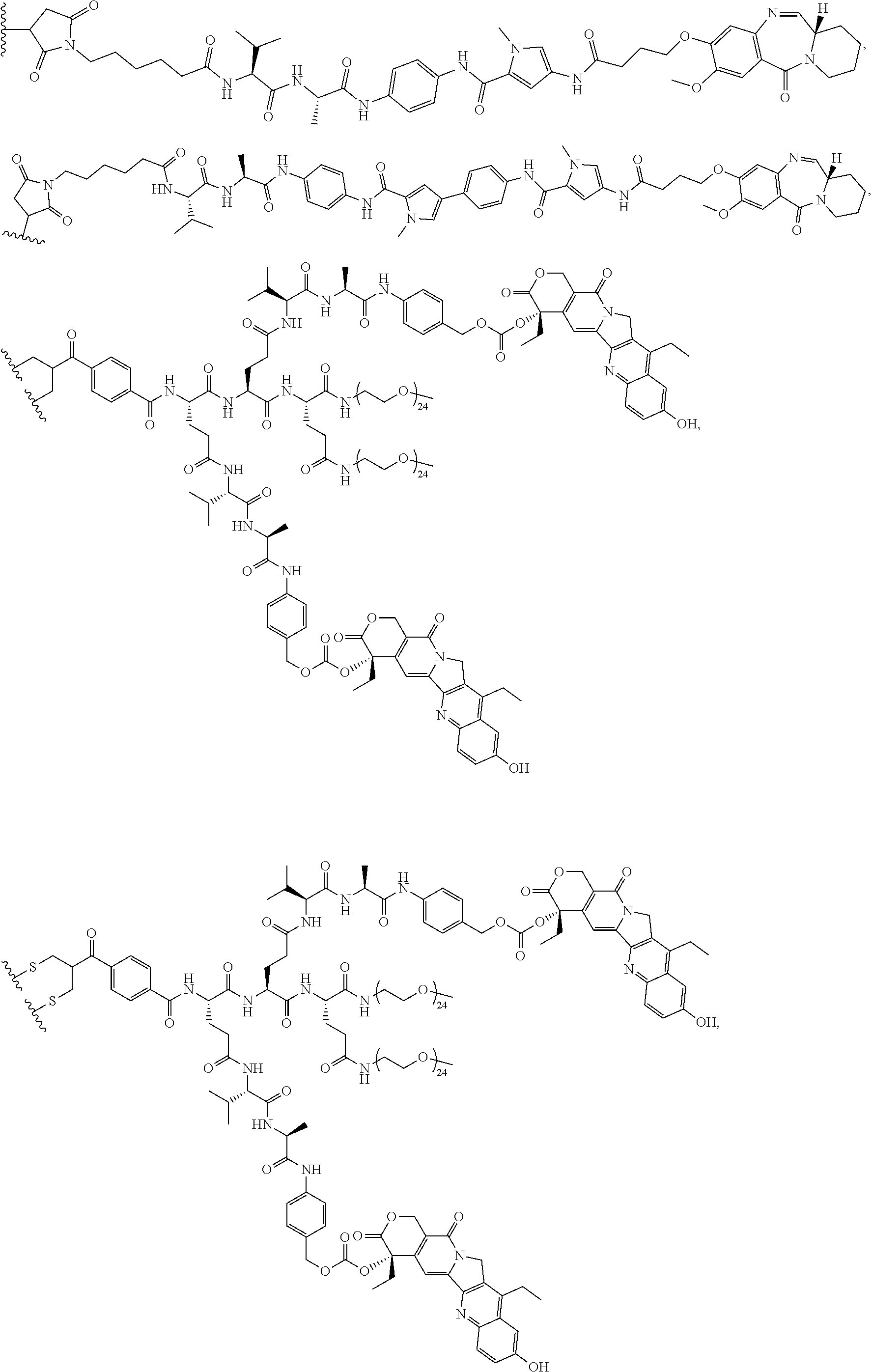

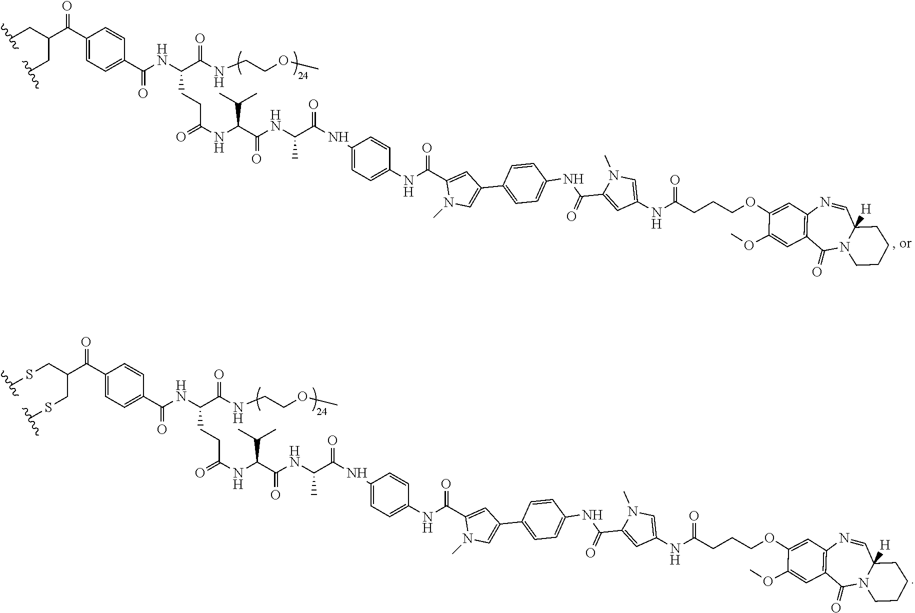

Embodiment 29. The antibody-drug conjugate of any one of embodiments 17-28, wherein n is an integer from 1 to 10, 2 to 8, or 4 to 8, optionally n is 1, 2, 3, 4, 5, 6, 7, 8, 9, or 10, optionally n is 4 or 8.

Embodiment 30. The antibody-drug conjugate of any one of embodiments 17-29, wherein the antibody or binding fragment thereof comprises:
(i) a heavy chain variable region (VH) that comprises:
   (a) a VH complementarity determining region one (CDRH1) comprising and/or consisting of the amino acid sequence of SEQ ID NO: 2,
   (b) a VH complementarity determining region two (CDRH2) comprising and/or consisting of the amino acid sequence of SEQ ID NO: 3, and
   (c) a VH complementarity determining region three (CDRH3) comprising and/or consisting of the amino acid sequence of SEQ ID NO: 4,
and (ii) a light chain variable region (VL) that comprises:
   (a) a VL complementarity determining region one (CDRL1) comprising and/or consisting of the amino acid sequence of SEQ ID NO: 6,
   (b) a VL complementarity determining region two (CDRL2) comprising and/or consisting of the amino acid sequence of SEQ ID NO: 7, and
   (c) a VL complementarity determining region three (CDRL3) comprising and/or consisting of the amino acid sequence of SEQ ID NO:8.

Embodiment 31. The antibody-drug conjugate of any one of embodiments 17-29, wherein the antibody or binding fragment thereof comprises:
(i) a heavy chain variable region (VH) that comprises:
   (a) a VH complementarity determining region one (CDRH1) comprising and/or consisting of the amino acid sequence of SEQ ID NO: 12,
   (b) a VH complementarity determining region two (CDRH2) comprising and/or consisting of the amino acid sequence of SEQ ID NO: 13, and
   (c) a VH complementarity determining region three (CDRH3) comprising and/or consisting of the amino acid sequence of SEQ ID NO: 14,
and (ii) a light chain variable region (VL) that comprises:
   (a) a VL complementarity determining region one (CDRL1) comprising and/or consisting of the amino acid sequence of SEQ ID NO: 6,
   (b) a VL complementarity determining region two (CDRL2) comprising and/or consisting of the amino acid sequence of SEQ ID NO: 7, and
   (c) a VL complementarity determining region three (CDRL3) comprising and/or consisting of the amino acid sequence of SEQ ID NO:8.

Embodiment 32. The antibody-drug conjugate of any one of embodiments 17-31, wherein the antibody or binding fragment thereof comprises a VH that comprises an amino acid sequence at least 90%, at least 91%, at least 92%, at least 93%, at least 94%, at least 95%, at least 96%, at least 97%, at least 98%, at least 99%, or 100% identical to the amino acid sequence of SEQ ID NO: 1, and a VL that comprises an amino acid sequence at least 90%, at least 91%, at least 92%, at least 93%, at least 94%, at least 95%, at least 96%, at least 97%, at least 98%, at least 99%, or 100% identical to the amino acid sequence of SEQ ID NO: 5.

Embodiment 33. The antibody-drug conjugate of embodiment 32, wherein the antibody or binding fragment thereof comprises a VH that comprises the amino acid sequence of SEQ ID NO: 1 and a VL that comprises the amino acid sequence of SEQ ID NO: 5.

Embodiment 34. The antibody-drug conjugate of any one of embodiments 17-33, wherein the antibody or binding fragment thereof comprises a heavy chain comprising an amino acid sequence at least 90%, at least 91%, at least 92%, at least 93%, at least 94%, at least 95%, at least 96%, at least 97%, at least 98%, at least 99%, or 100% identical to the amino acid sequence of SEQ ID NO: 1 and a light chain comprising an amino acid sequence at least 90%, at least 91%, at least 92%, at least 93%, at least 94%, at least 95%, at least 96%, at least 97%, at least 98%, at least 99%, or 100% identical to the amino acid sequence of SEQ ID NO: 5.

Embodiment 35. The antibody-drug conjugate of embodiment 34, wherein the antibody or binding fragment thereof comprises a heavy chain that comprises the amino acid sequence of SEQ ID NO: 1 and a light chain that comprises the amino acid sequence of SEQ ID NO: 5.

Embodiment 36. The antibody-drug conjugate of any one of embodiments 17-35, wherein the antibody-drug conjugate has a drug-to-antibody ratio (DAR) ranging from about 1 to about 10, optionally wherein the DAR is about 1, about 2, about 3, about 4, about 5, about 6, about 7, about 8, about 9, or about 10, optionally DAR is about 4, optionally DAR is about 8.

Embodiment 37. A pharmaceutical composition comprising the antibody drug conjugate of any one of embodiments 17-36; and a pharmaceutically acceptable carrier.

Embodiment 38. A method of treating a cancer comprising administering to a subject in need thereof a therapeutically effective amount of the antibody drug conjugate of any one of embodiments 16-35, or the pharmaceutical composition of embodiment 36.

Embodiment 39. The method of embodiment 37, wherein the cancer is selected from the group consisting of pancreatic cancer, breast cancer, prostate cancer, lymphoma, skin cancer, colon cancer, melanoma, malignant melanoma, ovarian cancer, brain cancer, primary brain carcinoma, head-neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, chronic lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, or retinoblastoma, and the like. In other embodiments, the cancer is acoustic neuroma, adenocarcinoma, angiosarcoma, astrocytoma, basal cell carcinoma, bile duct carcinoma, bladder carcinoma, brain cancer, breast cancer, triple-negative breast cancer (TNBC), bronchogenic carcinoma, cervical cancer, chordoma, choriocarcinoma, colon cancer, colorectal cancer, craniopharyngioma, cystadenocarcinoma, embryonal carcinoma, endotheliocarcinoma, ependymoma, epithelial carcinoma, esophageal cancer, Ewing's tumor, fibrosarcoma, gastric cancer, glioblastoma multiforme, glioma, head and neck cancer, hemangioblastoma, hepatoma, kidney cancer, leiomyosarcoma, liposarcoma, lung cancer, lymphangioendotheliosarcoma, lymphangiosarcoma, medullary carcinoma, medulloblastoma, melanoma, meningioma, mesothelioma, myxosarcoma, nasal cancer, neuroblastoma, oligodendroglioma, oral cancer, osteogenic sarcoma, ovarian cancer, pancreatic cancer, papillary adenocarcinoma, papillary carcinoma, pinealoma, prostate cancer, rabdomyosarcoma, rectal cancer, renal cell carcinoma, retinoblastoma, sarcoma, sebaceous gland carcinoma, seminoma, skin cancer, squamous cell carcinoma, stomach cancer, sweat gland carcinoma, synovioma, testicular cancer, small cell lung carcinoma, throat cancer, uterine cancer, Wilm's tumor, blood cancer, acute erythroleukemic leukemia, acute lymphoblastic B-cell leukemia, acute lymphoblastic T-cell leukemia, acute lymphoblastic leukemia, acute megakaryoblastic leukemia, acute monoblastic leukemia, acute myeloblastic leukemia, acute myelomonocytic leukemia, acute nonlymphocytic leukemia, acute promyelocytic leukemia, acute undifferentiated leukemia, chronic lymphocytic leukemia, chronic myelocytic leukemia, hairy cell leukemia, multiple myeloma, heavy chain disease, Hodgkin's disease, multiple myeloma, non-Hodgkin's lymphoma, polycythemia vera, or Waldenstrom's macroglobulinemia.

Embodiment 40. The method of embodiment 37 or 38, wherein the cancer in triple-negative breast cancer (TNBC).

Embodiment 41. An antibody-drug conjugate having formula (I):

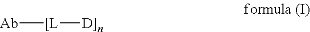

wherein in formula (I):
Ab comprises an antibody or antibody-binding fragment, wherein the antibody or binding fragment thereof specifically binds CUB Domain-Containing Protein-1 (CDCP1);
D comprises a drug moiety;
n is an integer from 1 to 20; and
L has the formula:

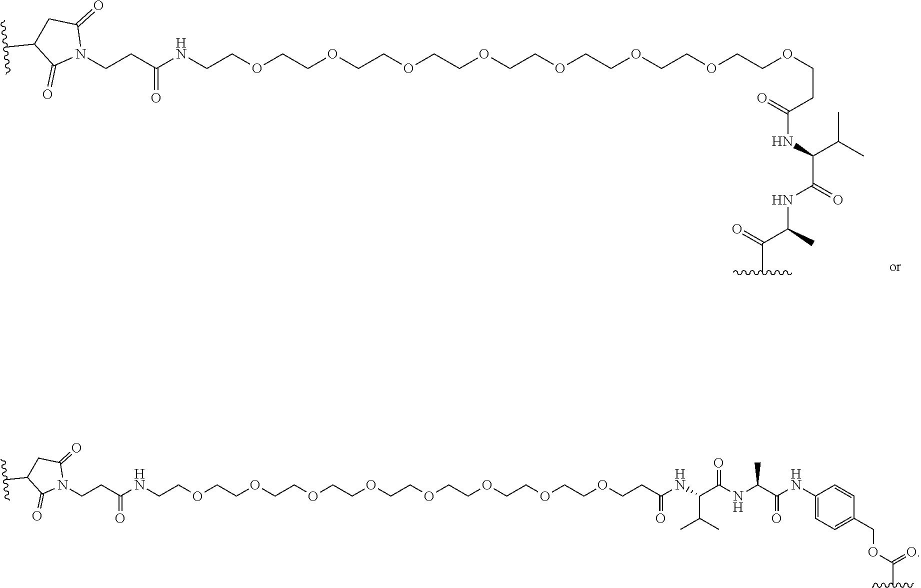 [SMILES: *C(=O)[C@H](C)NC(=O)[C@@H](NC(=O)CCOCCOCCOCCOCCOCCOCCOCCOCCNC(=O)CCN1C(=O)CC(*)C1=O)C(C)C ; *C(=O)OCc1ccc(NC(=O)[C@H](C)NC(=O)[C@@H](NC(=O)CCOCCOCCOCCOCCOCCOCCOCCOCCNC(=O)CCN2C(=O)CC(*)C2=O)C(C)C)cc1]

Embodiment 42. The antibody-drug conjugate of embodiment 41, wherein the antibody or binding fragment thereof comprises:
(i) a heavy chain variable region (VH) that comprises:
   (a) a VH complementarity determining region one (CDRH1) comprising and/or consisting of the amino acid sequence of SEQ ID NO: 2,
   (b) a VH complementarity determining region two (CDRH2) comprising and/or consisting of the amino acid sequence of SEQ ID NO: 3, and
   (c) a VH complementarity determining region three (CDRH3) comprising and/or consisting of the amino acid sequence of SEQ ID NO: 4,
and (ii) a light chain variable region (VL) that comprises:
   (a) a VL complementarity determining region one (CDRL1) comprising and/or consisting of the amino acid sequence of SEQ ID NO: 6,
   (b) a VL complementarity determining region two (CDRL2) comprising and/or consisting of the amino acid sequence of SEQ ID NO: 7, and
   (c) a VL complementarity determining region three (CDRL3) comprising and/or consisting of the amino acid sequence of SEQ ID NO:8.

Embodiment 43. The antibody-drug conjugate of embodiment 41, wherein the antibody or binding fragment thereof comprises:
(i) a heavy chain variable region (VH) that comprises:
   (a) a VH complementarity determining region one (CDRH1) comprising and/or consisting of the amino acid sequence of SEQ ID NO: 12,
   (b) a VH complementarity determining region two (CDRH2) comprising and/or consisting of the amino acid sequence of SEQ ID NO: 13, and
   (c) a VH complementarity determining region three (CDRH3) comprising and/or consisting of the amino acid sequence of SEQ ID NO: 14,
and (ii) a light chain variable region (VL) that comprises:
   (a) a VL complementarity determining region one (CDRL1) comprising and/or consisting of the amino acid sequence of SEQ ID NO: 6,
   (b) a VL complementarity determining region two (CDRL2) comprising and/or consisting of the amino acid sequence of SEQ ID NO: 7, and
   (c) a VL complementarity determining region three (CDRL3) comprising and/or consisting of the amino acid sequence of SEQ ID NO:8.

Embodiment 44. The antibody-drug conjugate of any one of embodiments 41-43, wherein the drug moiety comprises exatecan.

Embodiment 44A. The antibody-drug conjugate of any one of embodiments 41-43, wherein the linker L has the following formula:

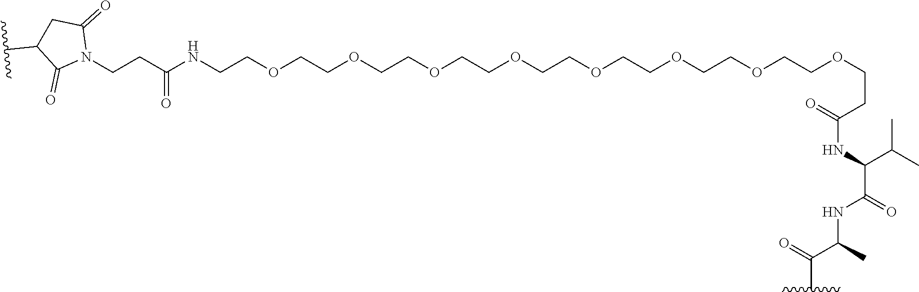 [SMILES: *C(=O)[C@H](C)NC(=O)[C@@H](NC(=O)CCOCCOCCOCCOCCOCCOCCOCCOCCNC(=O)CCN1C(=O)CC(*)C1=O)C(C)C]

Embodiment 45. The antibody-drug conjugate of any one of embodiments 41-43, wherein the drug moiety comprises Dxd.

Embodiment 46. The antibody-drug conjugate of any one of embodiments 41-43, wherein the drug moiety comprises monomethyl auristatin E (MMAE).

Embodiment 47. The antibody-drug conjugate of any one of embodiments 41-43, wherein the drug moiety comprises a pyrridinobenzodiazepine (PDD), optionally wherein the PDD comprises or has the formula:

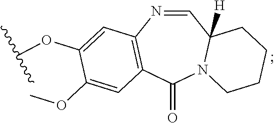 [SMILES: *Oc1cc2c(cc1OC)C(=O)N1CCCC[C@H]1C=N2]

optionally wherein the PDD comprises or has the formula:

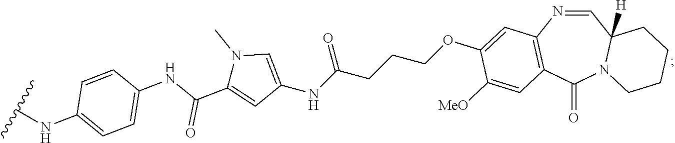 [SMILES: *Nc1ccc(NC(=O)c2cc(NC(=O)CCCOc3cc4c(cc3OC)C(=O)N3CCCC[C@H]3C=N4)cn2C)cc1]

optionally wherein the PDD comprises or has the formula:

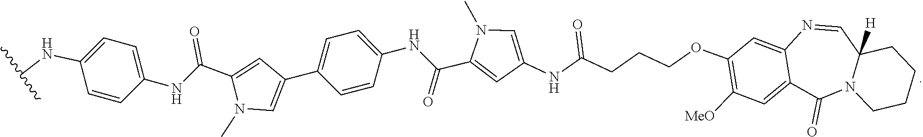 [SMILES: *Nc1ccc(NC(=O)c2cc(-c3ccc(NC(=O)c4cc(NC(=O)CCCOc5cc6c(cc5OC)C(=O)N5CCCC[C@H]5C=N6)cn4C)cc3)cn2C)cc1]

Embodiment 48. The antibody-drug conjugate of any one of embodiments 41-43, wherein the drug moiety comprises Sn-38.

Embodiment 49. The antibody-drug conjugate of any one of embodiments 41-48, wherein the antibody-drug conjugate has a drug-to-antibody ratio (DAR) ranging from about 1 to about 10, optionally wherein the DAR is about 1, about 2, about 3, about 4, about 5, about 6, about 7, about 8, about 9, or about 10, optionally DAR is about 4, optionally DAR is about 8.

Embodiment 50. An antibody-drug conjugate having formula (I):

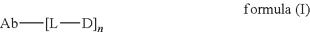

wherein in formula (I):
Ab comprises an antibody or antibody-binding fragment, wherein the antibody or binding fragment thereof specifically binds CUB Domain-Containing Protein-1 (CDCP1);
D comprises a drug moiety;
n is an integer from 1 to 20; and
L has the formula:

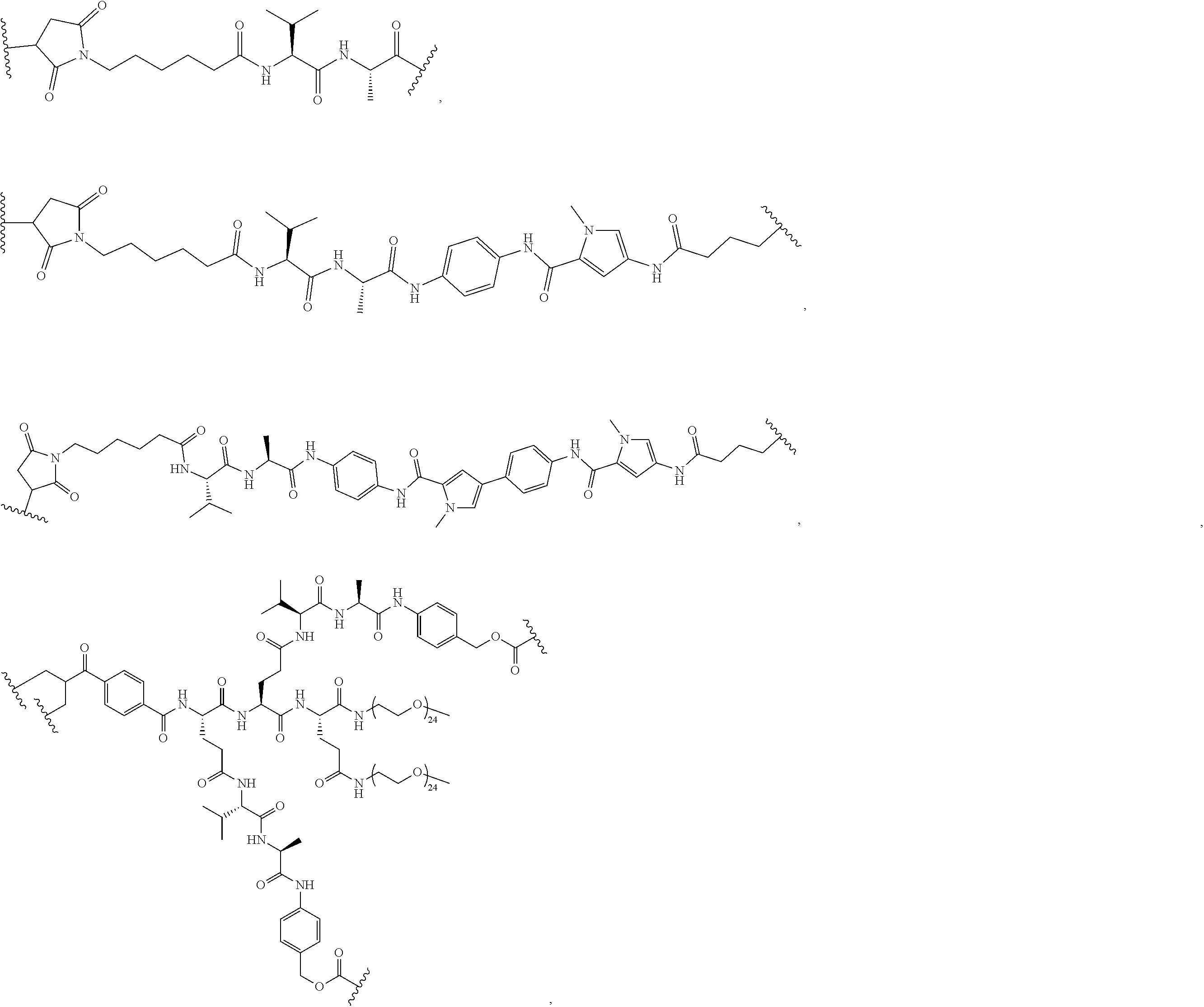

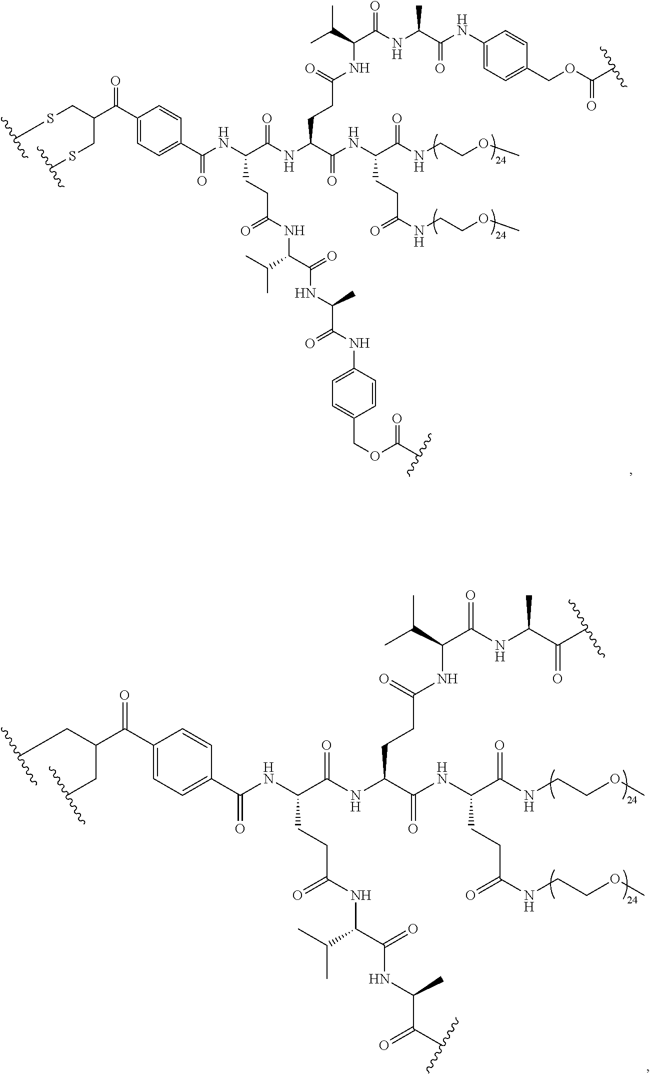

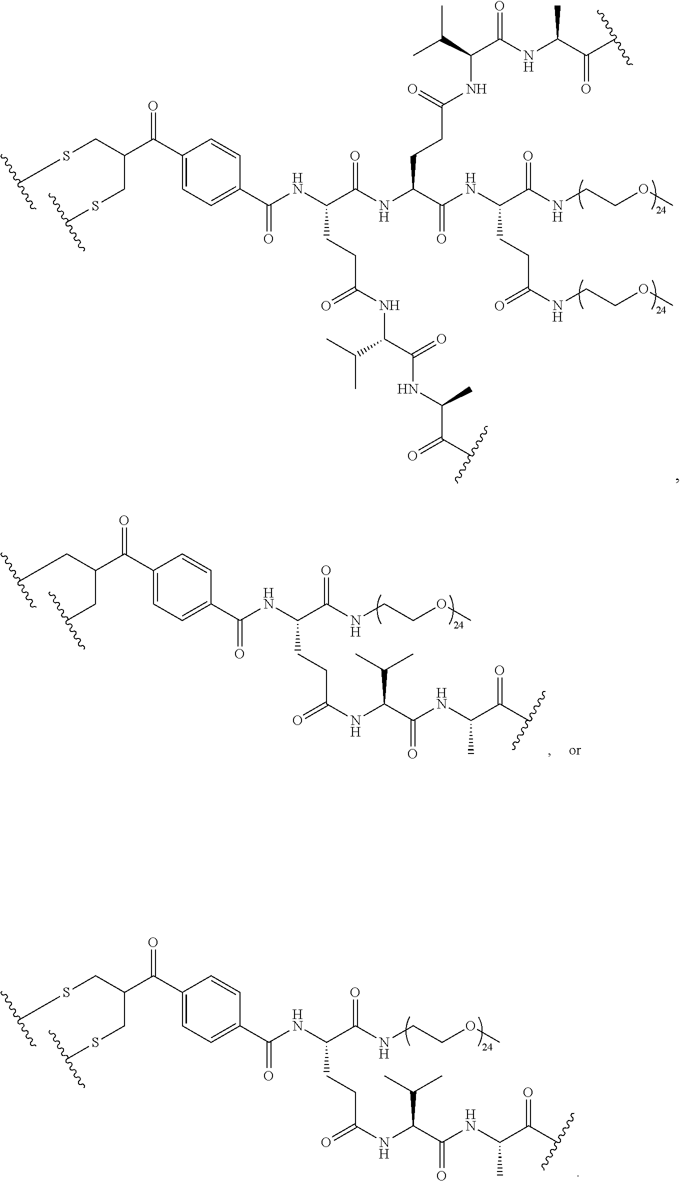

Embodiment 51. The antibody-drug conjugate of embodiment 49, wherein the antibody or binding fragment thereof comprises:
(i) a heavy chain variable region (VH) that comprises:
   (a) a VH complementarity determining region one (CDRH1) comprising and/or consisting of the amino acid sequence of SEQ ID NO: 2,
   (b) a VH complementarity determining region two (CDRH2) comprising and/or consisting of the amino acid sequence of SEQ ID NO: 3, and
   (c) a VH complementarity determining region three (CDRH3) comprising and/or consisting of the amino acid sequence of SEQ ID NO: 4,
and (ii) a light chain variable region (VL) that comprises:
   (a) a VL complementarity determining region one (CDRL1) comprising and/or consisting of the amino acid sequence of SEQ ID NO: 6,
   (b) a VL complementarity determining region two (CDRL2) comprising and/or consisting of the amino acid sequence of SEQ ID NO: 7, and
   (c) a VL complementarity determining region three (CDRL3) comprising and/or consisting of the amino acid sequence of SEQ ID NO:8.

Embodiment 52. The antibody-drug conjugate of embodiment 59, wherein the antibody or binding fragment thereof comprises:
(i) a heavy chain variable region (VH) that comprises:
   (a) a VH complementarity determining region one (CDRH1) comprising and/or consisting of the amino acid sequence of SEQ ID NO: 12,
   (b) a VH complementarity determining region two (CDRH2) comprising and/or consisting of the amino acid sequence of SEQ ID NO: 13, and
   (c) a VH complementarity determining region three (CDRH3) comprising and/or consisting of the amino acid sequence of SEQ ID NO: 14,
and (ii) a light chain variable region (VL) that comprises:
   (a) a VL complementarity determining region one (CDRL1) comprising and/or consisting of the amino acid sequence of SEQ ID NO: 6,
   (b) a VL complementarity determining region two (CDRL2) comprising and/or consisting of the amino acid sequence of SEQ ID NO: 7, and
   (c) a VL complementarity determining region three (CDRL3) comprising and/or consisting of the amino acid sequence of SEQ ID NO:8.

Embodiment 53. The antibody-drug conjugate of any one of embodiments 49-51, wherein the drug moiety comprises exatecan.

Embodiment 54. The antibody-drug conjugate of any one of embodiments 49-51, wherein the drug moiety comprises Dxd.

Embodiment 55. The antibody-drug conjugate of any one of embodiments 49-51, wherein the drug moiety comprises monomethyl auristatin E (MMAE).

Embodiment 56. The antibody-drug conjugate of any one of embodiments 49-51, wherein the drug moiety comprises a pyrridinobenzodiazepine (PDD), optionally wherein the PDD comprises or has the formula:

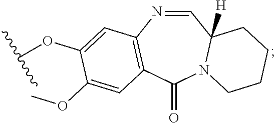 [SMILES: *Oc1cc2c(cc1OC)C(=O)N1CCCC[C@H]1C=N2]

optionally wherein the PDD comprises or has the formula:

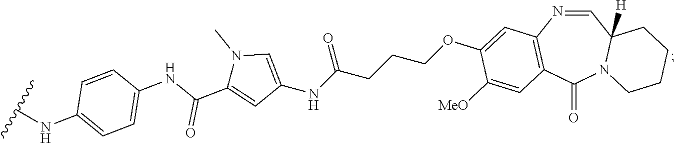 [SMILES: *Nc1ccc(NC(=O)c2cc(NC(=O)CCCOc3cc4c(cc3OC)C(=O)N3CCCC[C@H]3C=N4)cn2C)cc1]

optionally wherein the PDD comprises or has the formula:

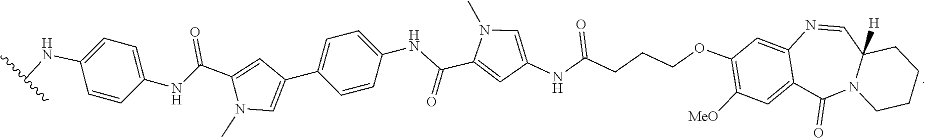 [SMILES: *Nc1ccc(NC(=O)c2cc(-c3ccc(NC(=O)c4cc(NC(=O)CCCOc5cc6c(cc5OC)C(=O)N5CCCC[C@H]5C=N6)cn4C)cc3)cn2C)cc1]

Embodiment 57. The antibody-drug conjugate of any one of embodiments 49-51, wherein the drug moiety comprises Sn-38.

Embodiment 58. The antibody-drug conjugate of any one of embodiments 49-56, wherein the antibody-drug conjugate has a drug-to-antibody ratio (DAR) ranging from about 1 to about 10, optionally wherein the DAR is about 1, about 2, about 3, about 4, about 5, about 6, about 7, about 8, about 9, or about 10, optionally DAR is about 4, optionally DAR is about 8.

Embodiment 59. An antibody-drug conjugate having formula (I):

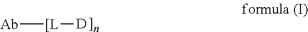

wherein in formula (I):
Ab comprises an antibody or antibody-binding fragment;
n is an integer from 1 to 20; and
L-D has the formula:

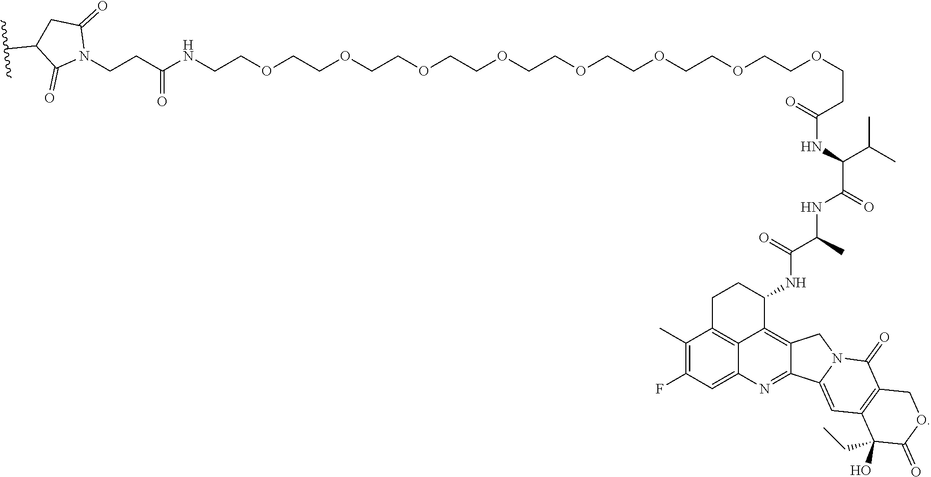 [SMILES: *C1CC(=O)N(CCC(=O)NCCOCCOCCOCCOCCOCCOCCOCCOCCC(=O)N[C@H](C(=O)N[C@@H](C)C(=O)N[C@H]2CCc3c(C)c(F)cc4nc5c(c2c34)Cn2c-5cc3c(c2=O)COC(=O)[C@]3(O)CC)C(C)C)C1=O]

Embodiment 60. An antibody-drug conjugate having formula (I):

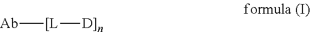

wherein in formula (I):
Ab comprises an antibody or antibody-binding fragment;
n is an integer from 1 to 20; and
L-D has the formula:

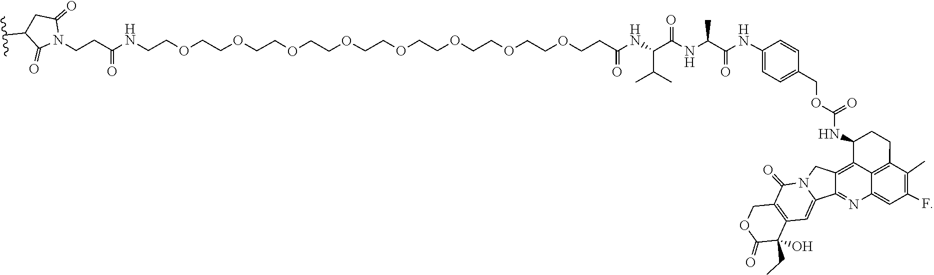 [SMILES: *C1CC(=O)N(CCC(=O)NCCOCCOCCOCCOCCOCCOCCOCCOCCC(=O)N[C@H](C(=O)N[C@@H](C)C(=O)Nc2ccc(COC(=O)N[C@H]3CCc4c(C)c(F)cc5nc6c(c3c45)Cn3c-6cc4c(c3=O)COC(=O)[C@]4(O)CC)cc2)C(C)C)C1=O]

Embodiment 61. An antibody-drug conjugate having formula (I):

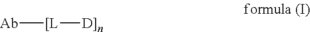

wherein in formula (I):
Ab comprises an antibody or antibody-binding fragment;
n is an integer from 1 to 20; and
L-D has the formula:

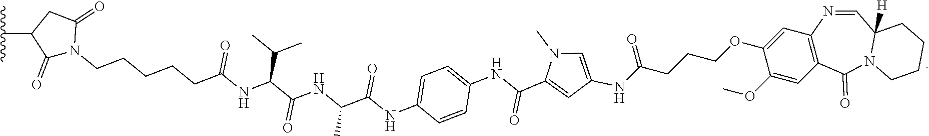 [SMILES: *C1CC(=O)N(CCCCCC(=O)N[C@H](C(=O)N[C@@H](C)C(=O)Nc2ccc(NC(=O)c3cc(NC(=O)CCCOc4cc5c(cc4OC)C(=O)N4CCCC[C@H]4C=N5)cn3C)cc2)C(C)C)C1=O]

Embodiment 62. An antibody-drug conjugate having formula (I):

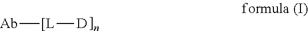

wherein in formula (I):
Ab comprises an antibody or antibody-binding fragment;
n is an integer from 1 to 20; and
L-D has the formula:

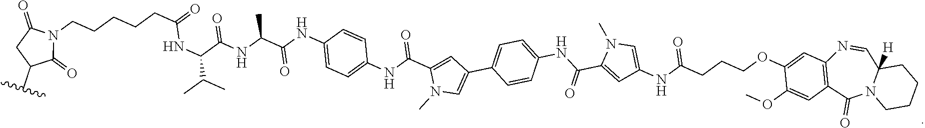 [SMILES: *C1CC(=O)N(CCCCCC(=O)N[C@H](C(=O)N[C@@H](C)C(=O)Nc2ccc(NC(=O)c3cc(-c4ccc(NC(=O)c5cc(NC(=O)CCCOc6cc7c(cc6OC)C(=O)N6CCCC[C@H]6C=N7)cn5C)cc4)cn3C)cc2)C(C)C)C1=O]

Embodiment 63. An antibody-drug conjugate having formula (I):

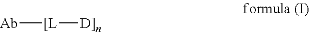

wherein in formula (I):
Ab comprises an antibody or antibody-binding fragment;
n is an integer from 1 to 20; and
L-D has the formula:

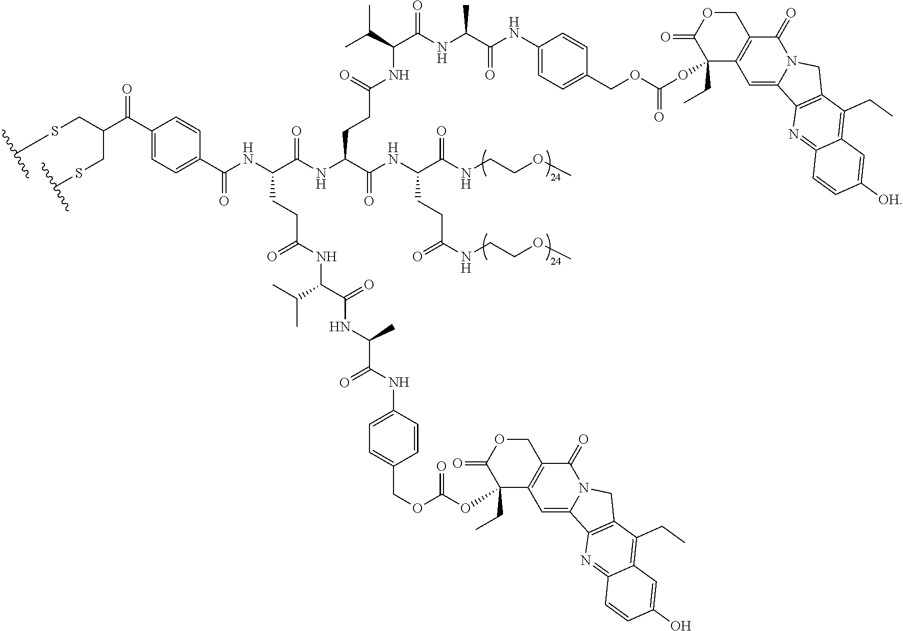

Embodiment 64. An antibody-drug conjugate having formula (I):

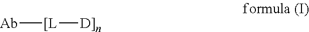

wherein in formula (I):
Ab comprises an antibody or antibody-binding fragment;
n is an integer from 1 to 20; and
L-D has the formula:

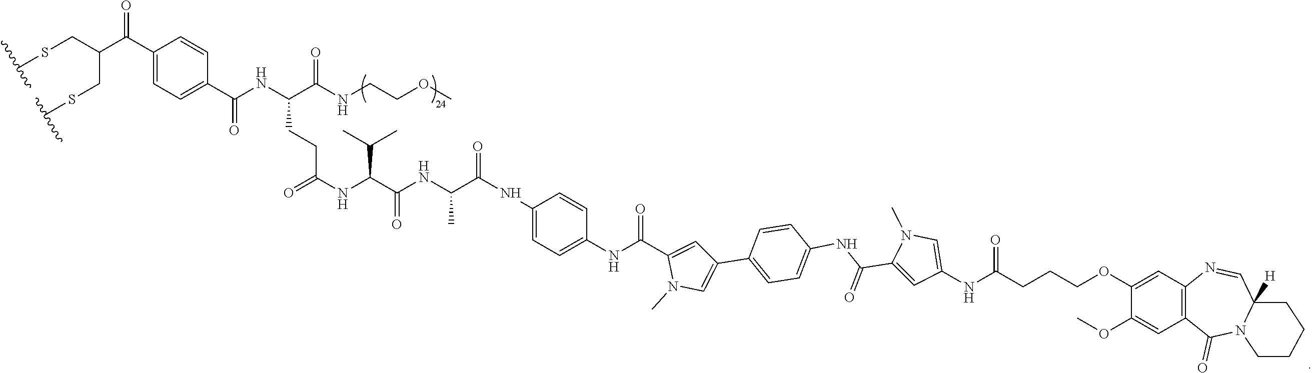

Embodiment 65. The antibody-drug conjugate of any one of embodiments 58-63, wherein the antibody-drug conjugate has a drug-to-antibody ratio (DAR) ranging from about 1 to about 10, optionally wherein the DAR is about 1, about 2, about 3, about 4, about 5, about 6, about 7, about 8, about 9, or about 10, optionally DAR is about 4, optionally DAR is about 8.

Embodiment 66. The antibody-drug conjugate of any one of embodiments 58-65, wherein n is an integer from 1 to 10, 2 to 8, or 4 to 8, optionally n is 1, 2, 3, 4, 5, 6, 7, 8, 9, or 10, optionally n is 4 or 8, optionally n is 4, optionally n is 8.

The following examples describe the disclosure in further detail. These examples are provided for illustrative purposes only, and should in no way be considered as limiting the disclosure.

### Examples

### Example 1: Chemical Synthesis

### General Remarks

All reagents and solvents were purchased from standard commercial suppliers and used as purchased. Anhydrous reactions were carried out under an inert atmosphere of argon using anhydrous solvents which were used as purchased, without further drying. Thin Layer Chromatography (TLC) was performed on silica gel aluminium plates (Merck 60, F₂₅₄), and flash column chromatography was carried out using a Biotage Isolera One (automated flash chromatography system), whilst monitoring by UV (254 nm) and TLC.

All Nuclear Magnetic Resonance (NMR) spectra were obtained at room temperature using a Bruker 600 MHz Ultrashield with Cryoprobe (Bruker Avance NEO console with Cryoplatform) or a Varian Mercury Vx Agilent 400 MHz spectrometer, for which chemical shifts are expressed in ppm relative to the solvent and coupling constants are expressed in Hz. Microwave reactions were carried out on a Biotage Initiator+ microwave synthesizer. High Resolution Mass Spectrometry (HRMS) was performed on a Thermo Scientific-Exactive HCD Orbitrap Mass Spectrometer. Yields refer to isolated material (homogeneous by TLC and NMR) unless otherwise stated and names are assigned according to IUPAC nomenclature.

Liquid Chromatography Mass Spectrometry (LCMS) analysis Methods A-C were performed on a Waters Alliance 2695 with water (A) and acetonitrile (B) comprising the mobile phases. Formic acid (0.1%) was added to both acetonitrile and water to ensure acidic conditions throughout the analysis. Function type: Diode array (535 scans). Column type: Monolithic C18 50×4.60 mm. Mass spectrometry data were collected using a Waters Micromass ZQ instrument coupled to the HPLC with a Waters 2996 PDA. Waters Micromass ZQ parameters used were: Capillary (kV), 3.38; Cone (V), 35; Extractor (V), 3.0; Source temperature (° C.), 100; De-solvation Temperature (° C.), 200; Cone flow rate (L/h), 50; De-solvation flow rate (L/h), 250. Gradient conditions are described as follows.

Method A (10 min): from 95% A/5% B to 50% B over 3 min. Then from 50% B to 80% B over 2 min. Then from 80% B to 95% B over 1.5 min and held constant for 1.5 min. This was then reduced to 5% B over 0.2 min and maintained to 5% B for 1.8 min. The flow rate was 0.5 mL/min, 200 μL was split via a zero dead volume T piece which passed into the mass spectrometer. The wavelength range of the UV detector was 220-400 nm.

Method B (5 min): from 95% A/5% B to 90% B over 3 min. Then from 90% B to 95% B over 0.5 min and held constant for 1 min. This was then reduced to 5% B over 0.5 min. The flow rate was 1.0 mL/min, 100 μL was split via a zero dead volume T piece which passed into the mass spectrometer. The wavelength range of the UV detector was 220-500 nm.

Method C (5 min): from 95% A/5% B, which was increased to 90% B over 3 min and to 95% B over a further 0.5 min. The gradient was then held at 95% B for 1 min and then returned to 5% B over 0.5 min. The total duration of the run was 5 minutes and the solvent flow rate was 1 mL/min, 100 μL was split via a zero dead volume T piece which passed into the mass spectrometer. The wavelength range of the UV detector was 220-500 nm.

Liquid Chromatography Mass Spectrometry (LCMS) analysis Methods D-G were performed on a Shimadzu LC-20AD series, Binary Pump, Diode Array Detector. Column type: Agilent Poroshell 120 EC-C18, 2.7 μm, 4.6×50 mm. Mobile phase: A: 0.05% formic acid in water (v/v); B: 0.05% formic acid in acetonitrile (v/v). Flow Rate: 1 mL/min at 25° C. Detector: 214 nm, 254 nm. Gradient stop time: 5 min. MS: 2020, Quadrupole LC/MS, Ion Source: API-ESI, TIC: 100-1300 m/z, Drying gas flow: 15 L/min, Nebulizer pressure: 1.5 L/min, Drying gas temperature: 250° C., Vcap: 4500V. Sample preparation: samples were dissolved in methanol at 1-10 μg/mL, then filtered through a 0.22 μm filter membrane. Injection volume: 1-10 μL. Gradient conditions are described as follows.

Method D (5 min): 20% A/80% B for 0.5 min, which was increased to 100% B over 3.5 min, then held at 100% B for 0.5 min. This was then returned to 20% A/80% B for 0.5 min.

Method E (5 min): 50% A/50% B for 0.5 min, which was increased to 100% B over 3.5 min, then held at 100% B for 0.5 min. This was then returned to 50% A/50% B for 0.5 min.

Method F (5 min): 85% A/15% B for 0.5 min, which was increased to 100% B over 3.5 min, then held at 100% B for 0.5 min. This was then returned to 85% A/15% B for 0.5 min.

Method G (5 min): 97% A/3% B for 0.5 min, which was increased to 30% A/70% B over 3.5 min, then to 100% B over 0.5 min. This was then returned to 97% A/3% B for 0.5 min.

Optical rotations were measured on an SGWzz-1 automatic Polarimeter (Shanghai Shen Guang Instrument Co., Ltd.) or Bellingham-Stanley ADP 440+ Polarimeter.

### 4-(Benzyloxy)-3-methoxybenzaldehyde (2)

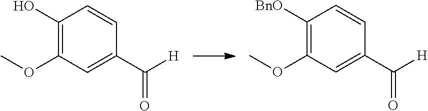 [SMILES: COc1cc(C=O)ccc1O ; COc1cc(C=O)ccc1OCc1ccccc1]

A mixture of compound vanillin (1) (200 g, 1.31 mol), benzyl bromide (236 g, 1.38 mol) and potassium carbonate (545 g, 3.94 mol) in methanol (1.20 L) was refluxed for 5 h. The reaction mixture was filtered, and the filtrate evaporated under reduced pressure to afford the title compound (271 g, 85%) as a pale yellow solid.

¹H NMR (400 MHz, CDCl₃) δ 9.83 (s, 1H), 7.47-7.35 (m, 6H), 7.33 (d, J=7.2 Hz, 1H), 6.98 (d, J=8.2 Hz, 1H), 5.24 (s, 2H), 3.94 (s, 3H); ¹³C NMR (100 MHz, CDCl₃) δ 191.0, 153.6, 150.1, 136.0, 130.3, 128.7, 128.2, 127.2, 126.6, 112.3, 109.3, 70.9, 56.1; MS (ES+): m/z=243 (M+H)⁺; LCMS (Method A): t_{R}=7.53 min.

### 4-(Benzyloxy)-5-methoxy-2-nitrobenzaldehyde (3)

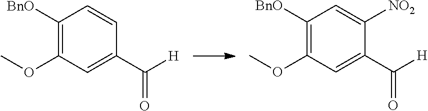 [SMILES: COc1cc(C=O)ccc1OCc1ccccc1 ; COc1cc(C=O)c([N+](=O)[O-])cc1OCc1ccccc1]

A solution of 4-(benzyloxy)-3-methoxybenzaldehyde (2) (130 g, 537 mmol) in trifluoroacetic acid (600 mL) was charged with a solution of potassium nitrate (65 g, 644 mmol), in trifluoroacetic acid (600 mL) dropwise at 0° C. The reaction mixture was stirred for 1 h and then diluted with water (2.40 L). The resulting precipitate was filtered and washed with cold water (500 mL×2) to afford the title compound (125 g, 81%) as a yellow solid.

¹H NMR (400 MHz, CDCl₃) δ 10.43 (s, 1H), 7.67 (s, 1H), 7.46-7.30 (m, 6H), 5.27 (s, 2H), 4.02 (s, 3H); ¹³C NMR (100 MHz, CDCl₃) δ 187.8, 153.7, 151.4, 134.85, 129.0, 128.9, 128.7, 127.6, 125.7, 110.0, 108.9, 71.6, 56.7; MS (ES−): m/z=286 (M−H)⁻; LCMS (Method A): t_{R}=7.87 min.

### 4-Hydroxy-5-methoxy-2-nitrobenzaldehyde (4)

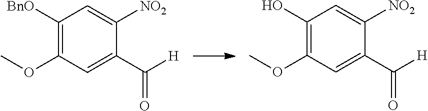 [SMILES: COc1cc(C=O)c([N+](=O)[O-])cc1OCc1ccccc1 ; COc1cc(C=O)c([N+](=O)[O-])cc1O]

A solution of 4-(benzyloxy)-5-methoxy-2-nitrobenzaldehyde (3) (100 g, 348 mmol) in glacial acetic acid (800 mL) was charged with an aqueous solution of hydrobromic acid (48% v/v, 88.0 mL, 522 mmol) and heated to 85° C., with stirring for 1 h, after which the reaction was judged to be complete by TLC. After allowing the resulting mixture to cool to room temperature, it was then diluted in water (1.60 L), and the resulting precipitate filtered, and washed with cold water (100 mL×3) to give the title compound (50.0 g, 73%) as a yellow solid, which was used immediately in the subsequent step without further purification.

¹H NMR (400 MHz, DMSO-d₆) δ 11.11 (br s, 1H), 10.15 (br s, 1H), 7.50 (s, 1H), 7.35 (s, 1H), 3.94 (s, 3H); MS (ES−): m/z=196 (M−H)⁻; LCMS (Method B): t_{R}=2.55 min.

### 5-Methoxy-2-nitro-4-((triisopropylsilyl)oxy)benzaldehyde (5)

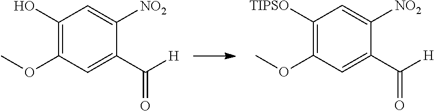

A mixture of 4-hydroxy-5-methoxy-2-nitrobenzaldehyde (4) (50.0 g, 254 mmol), triisopropylsilyl chloride (59.7 mL, 279 mmol) and imidazole (51.8 g, 761 mmol) was heated and stirred at 100° C. for 30 min. The reaction mixture was poured onto ice-water and extracted with ethyl acetate (500 mL×3). The organic extract was dried over sodium sulfate, filtered and concentrated under reduced pressure. The resulting residue was purified by flash column chromatography (silica), eluting with ethyl acetate/petroleum spirit, 40-60° C. (5%) to give the title compound (57.5 g, 64%) as a yellow solid.

¹H NMR (400 MHz, CDCl₃) δ 10.42 (s, 1H), 7.59 (s, 1H), 7.40 (s, 1H), 3.95 (s, 3H), 1.33-1.24 (m, 3H), 1.07 (s, 18H).

### 5-Methoxy-2-nitro-4-((triisopropylsilyl)oxy)benzoic acid (6)

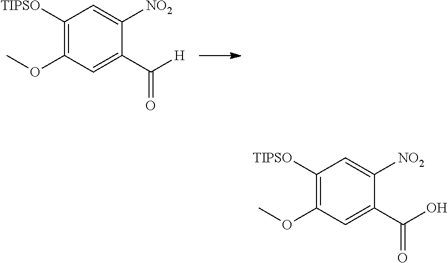

A solution of sodium chlorite (80%, 46.0 g, 407 mmol) and sodium phosphate monobasic dihydrate (35.5 g, 228 mmol) in water (200 mL) was added to a solution of 5-methoxy-2-nitro-4-((triisopropylsilyl)oxy)benzaldehyde (5) (57.5 g, 163 mmol) in tetrahydrofuran (800 mL) at room temperature. Hydrogen peroxide (30% w/w, 235 mL, 2.28 mol) was immediately added to the vigorously stirred biphasic mixture. The starting material dissolved, and the temperature of the reaction mixture rose to 45° C. After 30 min, the reaction was judged to have completed by TLC. The mixture was subsequently acidified to pH=3-4 with citric acid and extracted with ethyl acetate (500 mL×3). The combined organic extracts were washed with water (150 mL) and brine (150 mL), dried over anhydrous sodium sulfate, filtered and concentrated in vacuo. The residue was then purified by flash column chromatography (silica), eluting with ethyl acetate/petroleum spirit, 40-60° C. (10%) then methanol/dichloromethane (10%) to afford the title compound (38.0 g, 63%) as a yellow oil.

¹H NMR (400 MHz, CDCl₃) δ 9.81 (s, 1H), 7.35 (s, 1H), 7.25 (s, 1H), 3.91 (s, 3H), 1.26 (q, J=7.4 Hz, 3H), 1.09 (d, J=7.4 Hz, 18H); MS (ES−): m/z=368 (M−H)⁻; LCMS (Method D): t_{R}=4.75 min.

### (S)-(2-(Hydroxymethyl)piperidin-1-yl)(5-methoxy-2-nitro-4 ((triisopropylsilyl)oxy)phenyl)methanone (7)

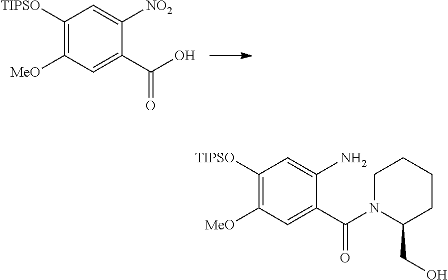

A solution of 5-methoxy-2-nitro-4-((triisopropylsilyl)oxy)benzoic acid (6) (28.0 g, 75.8 mmol), HATU (31.7 g, 83.4 mmol) and dry triethylamine (44 mL) in dry dichloromethane (300 mL) was stirred at room temperature for 30 min. (S)-Piperidin-2-ylmethanol (11.4 g, 98.5 mmol) was added and the reaction mixture was stirred at room temperature for 2 h. The reaction mixture was partitioned between dichloromethane (500 mL×2) and water (100 mL). The combined organic extracts were then dried over sodium sulfate, filtered and concentrated in vacuo. The resulting residue was purified by column chromatography (silica), eluting with ethyl acetate/petroleum spirit, 40-60° C. (from 50% to 75%), to give the title compound (20.0 g, 57%) as a yellow solid.

¹H NMR (400 MHz, CDCl₃) mixture of rotamers, δ 7.68-7.65 (m, 1H), 7.03-6.65 (m, 1H), 5.04-4.69 (m, 1H), 4.12-4.05 (m, 0.4H), 4.01-3.95 (m, 0.5H), 3.92-3.89 (m, 2.6H), 3.83-3.74 (m, 1.5H), 3.64-3.59 (m, 0.4H), 3.45-3.40 (m, 0.3H), 3.21-3.01 (m, 1.4H), 2.87-2.79 (m, 0.4H), 1.97-1.94 (m, 0.6H), 1.88-1.77 (m, 0.6H), 1.73-1.62 (m, 3H), 1.56-1.44 (m, 2H), 1.29-1.24 (m, 3H), 1.09 (d, J=7.3 Hz, 18H); MS (ES+): m/z=467 (M+H)⁺; LCMS (Method B): t_{R}=4.75 min.

### (S)-(2-Amino-5-methoxy-4-((triisopropylsilyl)oxy)phenyl)(2-(hydroxymethyl)piperidin-1-yl)methanone (8)

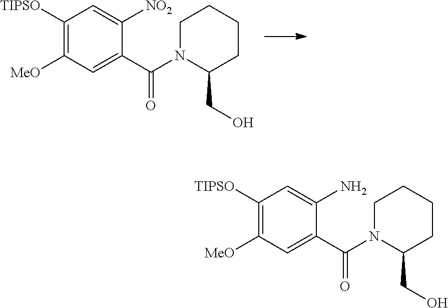

A solution of (S)-(2-(hydroxymethyl)piperidin-1-yl)(5-methoxy-2-nitro-4 ((triisopropylsilyl)oxy)phenyl)methanone (7) (1.00 g, 2.14 mmol) in tetrahydrofuran (5 mL) was charged with palladium on activated charcoal (10 wt. % basis, 100 mg), ammonium formate (1.10 g, 17.1 mmol) and water (1 mL), and stirred at room temperature, under argon, for 2 h. The resulting mixture was filtered through celite, the filter cake was washed with ethyl acetate (50 mL) and water (50 mL) and the filtrate separated. The organic phase was then extracted with brine (50 mL×2), and dried over magnesium sulfate, filtered and concentrated in vacuo. Purification by flash column chromatography (silica), eluting with ethyl acetate/petroleum spirit, 40-60° C. (from 50% to 67%), gave the title compound (892 mg, 95%) as a yellow solid.

¹H NMR (400 MHz, CDCl₃) δ 6.67 (s, 1H), 6.30 (s, 1H), 4.00-3.81 (m, 4H), 3.72 (s, 3H), 3.57 (s, 1H), 3.08 (s, 1H), 1.68-1.64 (m, 4H), 1.57-1.43 (m, 2H), 1.28-1.17 (m, 3H), 1.08 (d, J=7.4 Hz, 18H); ¹³C NMR (100 MHz, CDCl₃) δ 171.8, 147.9, 143.7, 133.2, 112.5, 110.0, 109.5, 68.7, 61.0, 56.4, 30.9, 25.8, 19.9, 17.9, 12.9; MS (ES+): m/z=437 (M+H)⁺; LCMS (Method C): t_{R}=4.07 min.

### Allyl(S)-(2-(2-(hydroxymethyl)piperidine-1-carbonyl)-4-methoxy-5-((triisopropylsilyl)oxy)phenyl)carbamate (9)

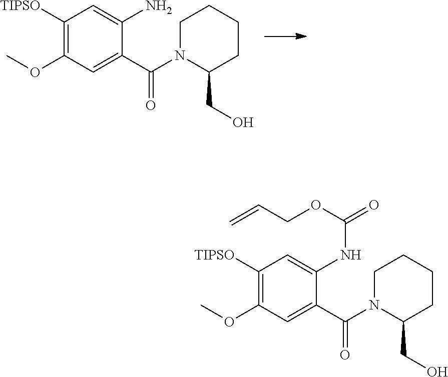

A solution of (S)-(2-amino-5-methoxy-4-((triisopropylsilyl)oxy)phenyl)(2-(hydroxymethyl)piperidin-1-yl)methanone (8) (892 mg, 2.04 mmol) in dichloromethane (4 mL) was cooled to −10° C. and charged with pyridine (380 μL) and allyl chloroformate (228 μL, 2.14 mmol), dropwise under argon. After 35 min, the reaction mixture was diluted with dichloromethane (10 mL), then extracted with a saturated aqueous solution of copper sulfate (10 mL×2) and brine (10 mL), dried over magnesium sulfate, filtered and concentrated in vacuo. Purification by flash column chromatography (silica), eluting with ethyl acetate/petroleum spirit, 40-60° C. (from 50% to 75%), gave the title compound (907 mg, 85%) as a pale yellow solid.

¹H NMR (400 MHz, CDCl₃) δ 8.08 (s, 1H), 7.62 (s, 1H), 6.75 (s, 1H), 5.92 (ddt, J=17.2, 10.7, 5.5 Hz, 1H), 5.32 (dt, J=17.3, 1.7 Hz, 1H), 5.20 (dt, J=10.6, 1.4 Hz, 1H), 4.61 (dt, J=5.5, 1.5 Hz, 2H), 3.88 (t, J=10.7 Hz, 1H), 3.76 (s, 3H), 3.61-3.57 (m, 1H), 3.20-3.02 (m, 2H), 2.03 (s, 1H), 1.65-1.62 (m, 3H), 1.53-1.40 (m, 2H), 1.29-1.24 (m, 4H), 1.11-1.08 (m, 18H); MS (ES+): m/z=522 (M+H)⁺; LCMS (Method A): t_{R}=9.62 min.

### Allyl (6aS)-6-hydroxy-2-methoxy-12-oxo-3-((triisopropylsilyl)oxy)-6,6a,7,8,9,10-hexahydrobenzo[e]pyrido[1,2-a][1,4]diazepine-5(12H)-carboxylate (10)

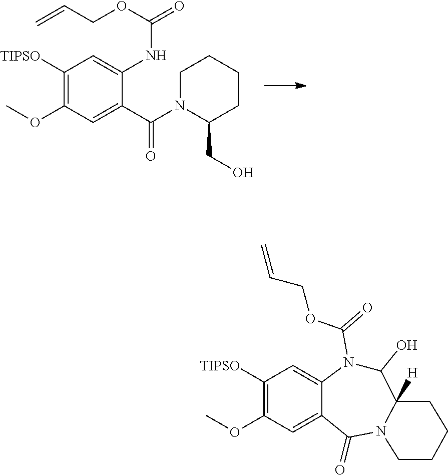

A solution of allyl (S)-(2-(2-(hydroxymethyl)piperidine-1-carbonyl)-4-methoxy-5-((triisopropylsilyl)oxy)phenyl)carbamate (9) (17.0 g, 32.7 mmol) in dichloromethane (150 mL) was charged with (diacetoxyiodo)benzene (12.6 g, 39.2 mmol) and 2,2,6,6-tetramethylpiperidine 1-oxyl (510 mg, 3.30 mmol), and stirred at room temperature for 16 h. The resulting mixture was then diluted with dichloromethane (350 mL), and sequentially washed with a saturated aqueous solution of sodium metabisulfite (100 mL) and a saturated aqueous solution of sodium hydrogen carbonate (100 mL). The organic extract was then dried over sodium sulfate, filtered and concentrated in vacuo. The resulting residue was then purified by flash column chromatography (silica), eluting with ethyl acetate/petroleum spirit, 40-60° C. (from 25% to 50%), to give the title compound (13.0 g, 77%) as a pale yellow solid.

¹H NMR (400 MHz, CDCl₃) δ 7.13 (s, 1H), 6.65 (s, 1H), 5.90 (d, J=10.3 Hz, 1H), 5.76 (s, 1H), 5.14 (t, J=12.1 Hz, 2H), 4.59 (dd, J=13.1, 5.3 Hz, 1H), 4.44 (dd, J=12.9, 5.1 Hz, 1H), 4.34 (dt, J=13.5, 4.1 Hz, 1H), 3.83 (s, 3H), 3.77 (br, 1H), 3.45 (ddd, J=10.1, 5.9, 4.0 Hz, 1H), 3.10-2.99 (m, 1H), 2.09-1.98 (m, 1H), 1.82-1.67 (m, 2H), 1.67-1.56 (m, 3H), 1.28-1.15 (m, 3H), 1.06 (dd, J=7.4, 2.5 Hz, 18H); ¹³C NMR (100 MHz, CDCl₃) δ 169.2, 156.2, 150.6, 147.6, 131.9, 127.0, 125.7, 121.2, 118.2, 110.9, 82.3, 66.9, 55.5, 55.3, 38.6, 23.2, 23.0, 18.2, 17.8, 12.8; MS (ES+): m/z=519 (M+H)⁺; LCMS (Method A): t_{R}=8.67 min.

### Allyl (6aS)-2-methoxy-12-oxo-6-((tetrahydro-2H-pyran-2-yl)oxy)-3-((triisopropylsilyl)oxy)-6,6a,7,8,9,10-hexahydrobenzo[e]pyrido[1,2-a][1,4]diazepine-5 (12H)-carboxylate (11)

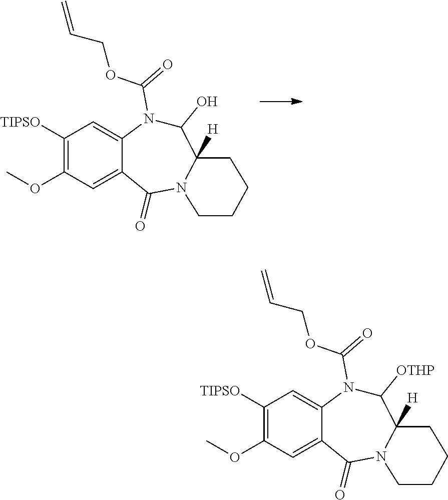

A solution of allyl (6aS)-6-hydroxy-2-methoxy-12-oxo-3-((triisopropylsilyl)oxy)-6,6a, 7,8,9,10-hexahydrobenzo[e]pyrido[1,2-a][1,4]diazepine-5(12H)-carboxylate (10) (14.0 g, 27.0 mmol) in tetrahydrofuran (130 mL) was charged with 3,4-dihydro-2H-pyran (24.6 g, 270 mmol) and p-toluenesulfonic acid monohydrate (140 mg, 0.76 mmol), and stirred for 18 h at room temperature. The resulting mixture was then diluted with ethyl acetate (360 mL) and washed with a saturated aqueous solution of sodium hydrogen carbonate (200 mL) and brine (100 mL). The organic phase was dried over sodium sulfate, filtered and concentrated in vacuo. Purification by flash column chromatography (silica), eluting with ethyl acetate/petroleum spirit, 40-60° C. (17%), gave the title compound (12.5 g, 77%) as a yellow oil.

¹H NMR (400 MHz, CDCl₃), 1:1 mixture of diastereomers, δ 7.13 (s, 0.4H), 7.10 (s, 0.5H), 6.90 (s, 0.5H), 6.52 (s, 0.4H), 6.15 (d, J=10.0 Hz, 0.4H), 5.98 (d, J=10.0 Hz, 0.5H), 5.80-5.68 (m, 1H), 5.17-4.94 (m, 3H), 4.64-4.21 (m, 3H), 3.91-3.85 (m, 1H), 3.83 (d, J=1.8 Hz, 3H), 3.66-3.39 (m, 2H), 3.14-3.00 (m, 1H), 2.08-1.87 (m, 1H), 1.83-1.33 (m, 12H), 1.26-1.19 (m, 3H), 1.08-1.05 (m, 18H); MS (ES+): m/z=603 (M+H)⁺; LCMS (Method A): t_{R}=9.95 min.

### Allyl (6aS)-3-hydroxy-2-methoxy-12-oxo-6-((tetrahydro-2H-pyran-2-yl)oxy)-6,6a,7,8,9,10-hexahydrobenzo[e]pyrido[1,2-a][1,4]diazepine-5(12H)-carboxylate (12)

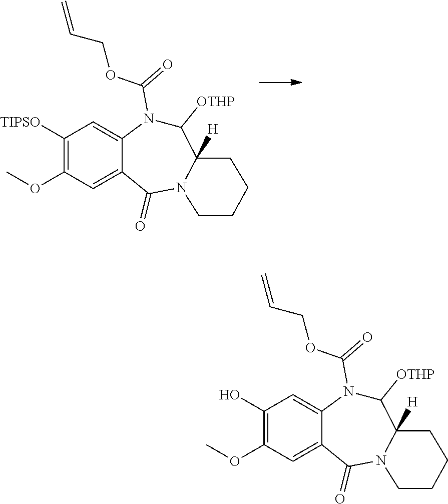

A solution of allyl (6aS)-2-methoxy-12-oxo-6-((tetrahydro-2H-pyran-2-yl)oxy)-3-((triisopropylsilyl)oxy)-6,6a,7,8,9,10-hexahydrobenzo[e]pyrido[1,2-a][1,4]diazepine-5(12H)-carboxylate (11) (10.5 g, 17.4 mmol) in tetrahydrofuran (33 mL) was charged with tetrabutylammonium fluoride (1 M in tetrahydrofuran, 26.1 mL, 26.1 mmol) at room temperature and stirred, to give an instantaneous orange colour. After 10 min, the reaction mixture was concentrated in vacuo, then immediately purified by flash column chromatography (silica), eluting with ethyl acetate/petroleum spirit, 40-60° C. (from 0% to 100%), to give the title compound (7.13 g, 92%) as a white solid.

¹H NMR (400 MHz, CDCl₃), mixture of diastereomers, δ 7.16 (s, 0.4H), 7.13 (s, 0.6H), 6.91 (br, 0.5H), 6.62 (br, 0.3H), 6.16 (d, J=10.1 Hz, 0.4H), 5.99 (d, J=10.1 Hz, 0.6H), 5.76 (ddd, J=15.8, 10.3, 5.0 Hz, 0.8H), 5.14-5.03 (m, 2H), 4.98 (br, 0.5H), 4.61 (dd, J=13.7, 4.9 Hz, 0.8H), 4.55-4.48 (m, 0.3H), 4.48 (br, 0.3H), 4.40 (dd, J=13.9, 5.2 Hz, 0.5H), 4.32-4.20 (m, 1H), 3.90 (s, 3H), 3.88-3.81 (m, 1H), 3.63 (br, 0.4H), 3.59-3.52 (m, 0.6H), 3.49-3.43 (m, 1H), 3.11-2.98 (m, 1H), 1.97-1.89 (m, 1H), 1.80-1.47 (m, 12H); ¹³C NMR (100 MHz, CDCl₃), mixture of diastereomers, δ 169.4, 169.2, 155.8, 147.9, 147.7, 146.6, 146.4, 132.2, 132.0, 128.4, 128.2, 125.9, 125.5, 117.0, 116.4, 115.8, 110.2, 109.8, 100.5, 95.3, 88.0, 84.1, 66.5, 66.3, 63.8, 63.2, 60.4, 56.2, 56.1, 55.4, 55.2, 38.8, 38.8, 31.0, 30.6, 25.2, 25.1, 23.22, 23.1, 23.0, 23.0, 20.1, 19.7, 18.4, 18.2; MS (ES+): m/z=447 (M+H)⁺; LCMS (Method A): t_{R}=6.87 min.

### Allyl (6aS)-2-methoxy-3-(4-methoxy-4-oxobutoxy)-12-oxo-6-((tetrahydro-2H-pyran-2-yl)oxy)-6,6a,7,8,9,10-hexahydrobenzo[e]pyrido[1,2-a][1,4]diazepine-5(12H)-carboxylate (13)

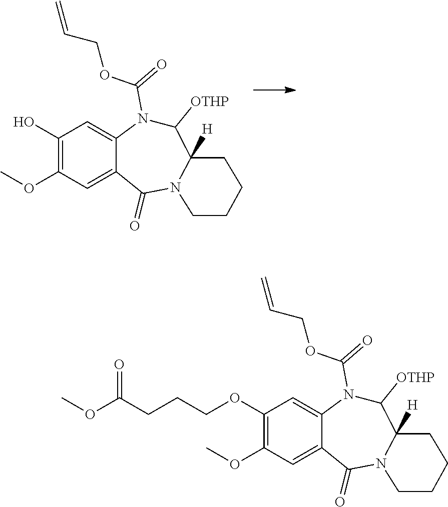

A solution of allyl (6aS)-3-hydroxy-2-methoxy-12-oxo-6-((tetrahydro-2H-pyran-2-yl)oxy)-6,6a,7,8,9,10-hexahydrobenzo[e]pyrido[1,2-a][1,4]diazepine-5(12H)-carboxylate (12) (1.0 g, 2.24 mmol) in N,N-dimethylformamide (5 mL) was charged with potassium carbonate (464 mg, 3.36 mmol) and methyl 4-bromobutanoate (296 μL, 2.35 mmol) and the resulting mixture was stirred rapidly at room temperature for 16 h. After diluting into ethyl acetate (100 mL), the solution was washed with brine (2×50 mL), dried over anhydrous magnesium sulfate, filtered, and concentrated in vacuo. Purification by flash column chromatography (silica), eluting with methanol/dichloromethane (from 0% to 2%) gave the title compound (1.02 g, 84%) as a cream solid (mixture of diastereomers).

¹H NMR (400 MHz, CDCl₃) δ 7.50 (s, 0.6H), 7.02 (s, 0.4H), 6.74 (s, 0.4H), 6.48 (s, 0.6H), 6.07 (d, J=9.8 Hz, 0.6H), 5.9 (d, J=10.2 Hz, 0.4H), 5.70-5.62 (m, 1H), 5.01-4.92 (m, 3H), 4.55-4.20 (m, 2H), 4.18-4.13 (m, 1H), 3.96-3.91 (m, 3H), 3.78 (s, 3H), 3.55 (s, 3H), 3.40-3.34 (m, 2H), 3.00-2.91 (m, 1H), 2.24 (t, J=7.0 Hz, 2H), 2.05-2.02 (m, 2H), 1.67-1.43 (m, 12H); ¹³C NMR (150 MHz, DMSO-d₆) δ 173.4, 168.5, 150.1, 149.2, 133.2, 127.6, 126.4, 116.9, 114.6, 110.8, 99.8, 94.7, 88.0, 83.9, 68.0, 66.0, 63.3, 62.5, 56.2, 55.4, 51.8, 38.6, 30.6, 30.2, 25.3, 24.3, 23.1, 19.7, 18.3; MS (ES+): m/z=547 (M+H)⁺; LCMS (Method A): t_{R}=7.70 min.

### 4-(((6aS)-5-((Allyloxy)carbonyl)-2-methoxy-12-oxo-6-((tetrahydro-2H-pyran-2-yl)oxy)-5,6,6a,7,8,9,10,12-octahydrobenzo[e]pyrido[1,2-a][1,4]diazepin-3-yl)oxy)butanoic acid (14)

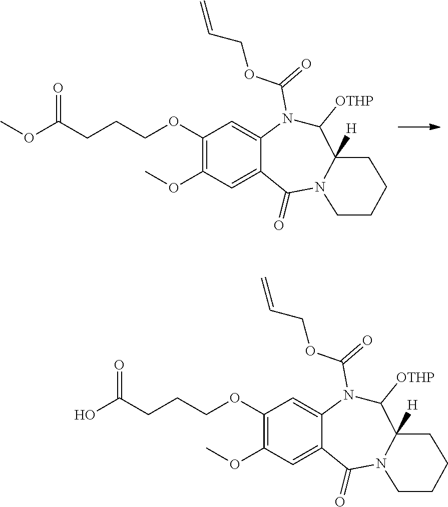

A solution of allyl (6aS)-2-methoxy-3-(4-methoxy-4-oxobutoxy)-12-oxo-6-((tetrahydro-2H-pyran-2-yl)oxy)-6,6a,7,8,9,10-hexahydrobenzo[e]pyrido[1,2-a][1,4]diazepin-5(12H)-carboxylate (13) (770 mg, 1.40 mmol) in 1,4-dioxane (10 mL) was charged with an aqueous solution of sodium hydroxide (1 M, 10.0 mL, 10.00 mmol) and the resulting mixture was stirred at room temperature for 2 h, before concentrating in vacuo. Water (20 mL) was then added and neutralisation was brought about by cautious addition of an aqueous solution of acetic acid (5 M, 10 mL, 50 mmol). After extracting with ethyl acetate (2×50 mL), the combined organic extracts were then washed with brine (50 mL), dried over sodium sulfate, filtered, and concentrated in vacuo to give the title compound (700 mg, 93%) as a yellow oil (mixture of diastereomers). The product was employed in the subsequent step without any further purification.

¹H NMR (400 MHz, DMSO-d₆) δ 12.15 (br. s., 1H), 7.03 (s, 0.6H), 7.01 (s, 0.4H), 6.86 (s, 0.6H), 6.78 (s, 0.4H), 6.01 (d, J=10.1, 0.6 H), 5.92 (d, J=9.8, 0.4H), 5.83-5.69 (m, 1H), 5.11-4.96 (m, 3H), 4.64-4.36 (m, 2H), 4.16-4.02 (m, 1H), 400-3.92 (m, 2H), 3.80 (s, 3H), 3.79-3.70 (m, 2H), 3.54-3.46 (m, 1H), 2.89-2.83 (m, 1H), 2.36 (t, J=7.2 Hz, 2H), 1.96-1.89 (m, 2H), 1.71-1.41 (m, 12H); ¹³C NMR (100 MHz, DMSO-d₆) δ 174.5, 174.4, 168.5, 168.5, 150.1, 149.1, 133.1, 127.6, 126.3, 114.5, 110.7, 109.1, 99.7, 84.4, 68.0, 67.9, 56.2, 52.9, 38.5, 30.6, 30.3, 30.2, 25.4, 25.3, 23.1, 23.0, 18.3; MS (ES+/−): m/z=533 (M+H)⁺, 531 (M−H)⁻; LCMS (Method A): t_{R}=6.98 min.

### tert-Butyl (1-methyl-5-((4-(2,2,2-trifluoroacetamido)phenyl)carbamoyl)-1H-pyrrol-3-yl)carbamate (15)

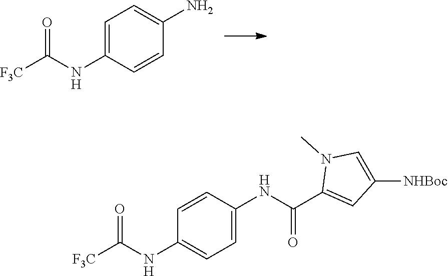 [SMILES: Nc1ccc(NC(=O)C(F)(F)F)cc1 ; Cn1cc(NC(=O)OC(C)(C)C)cc1C(=O)Nc1ccc(NC(=O)C(F)(F)F)cc1]

A solution of 4-((tert-butoxycarbonyl)amino)-1-methyl-1H-pyrrol-2-carboxylic acid (647 mg, 2.69 mmol), 4-(dimethylamino)pyridine (898 mg, 7.35 mmol) and N-(3-dimethylaminopropyl)-N′-ethylcarbodiimide hydrochloride (1.2 g, 6.25 mmol) in N,N-dimethylformamide (8 mL) was stirred for 30 min, after which N-(4-aminophenyl)-2,2,2-trifluoroacetamide (500 mg, 2.45 mmol) was then added. The resulting solution was stirred at room temperature for 18 h and then quenched with a saturated aqueous solution of sodium hydrogen carbonate (15 mL). After extracting with ethyl acetate (2×50 mL), the combined organic extracts were dried over magnesium sulfate and then concentrated in vacuo. The resulting residue was purified by flash column chromatography (silica), eluting with ethyl acetate/petroleum spirit, 40-60° C. (from 0% to 100%), to give the title compound (839 mg, 80%) as a cream solid.

¹H NMR (600 MHz, DMSO-d₆) δ 11.17 (s, 1H), 9.85 (s, 1H), 9.11 (s, 1H), 7.74 (d, J=9.0 Hz, 2H), 7.58 (d, J=9.0 Hz, 2H), 6.94 (s, 1H), 3.81 (s, 3H), 1.46 (s, 9H); ¹³C NMR (150 MHz, DMSO-d₆) δ 160.2, 153.3, 1375, 131.6, 123.1, 122.9, 121.8, 120.8, 118.4, 117.3, 115.4, 105.2, 78.8, 36.6, 28.7; MS (ES+): m/z=427 (M+H)⁺; LCMS (Method B): t_{R}=3.83 min.

### 4-Amino-1-methyl-N-(4-(2,2,2-trifluoroacetamido)phenyl)-1H-pyrrole-2-carboxamide hydrochloride (16)

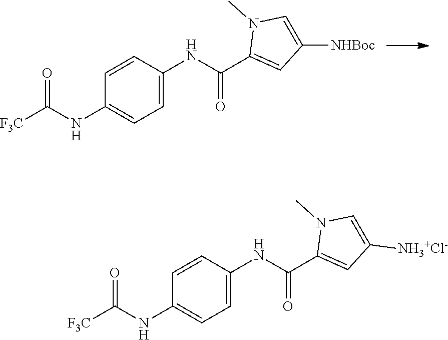

A solution of tert-butyl (1-methyl-5-((4-(2,2,2-trifluoroacetamido)phenyl)carbamoyl)-1H-pyrrol-3-yl)carbamate (15) (200 mg, 0.469 mmol) in dichloromethane/methanol (9:1, 1 mL) was charged with HCl (4 M in 1,4-dioxane, 1 mL) and stirred at room temperature for 20 min, before concentrating in vacuo. Diethyl ether (10 mL) was then added to the residue, before concentrating again, and then applying strong vacuum for 1 h, to give the title compound (170 mg, crude) as a white solid, which was used immediately in the subsequent step without further purification.

MS (ES+): m/z=327 (M+H)⁺; LCMS (Method A): t_{R}=4.80 min.

### Allyl (6aS)-2-methoxy-3-(4-((1-methyl-5-((4-(2,2,2-trifluoroacetamido)phenyl)carbamoyl)-1H-pyrrol-3-yl)amino)-4-oxobutoxy)-12-oxo-6-((tetrahydro-2H-pyran-2-yl)oxy)-6,6a,7,8,9,10-hexahydrobenzo[e]pyrido[1,2-a][1,4]diazepine-5(12H)-carboxylate (17)

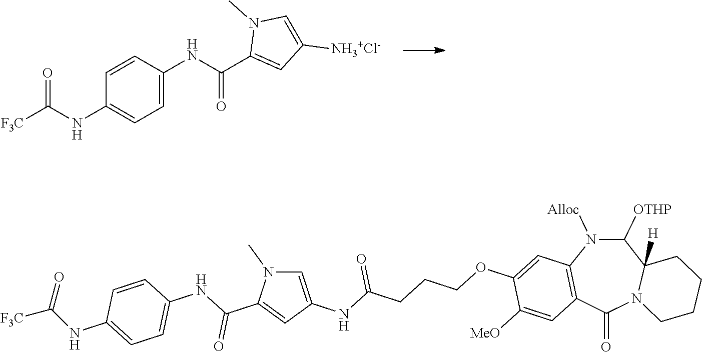

4-Amino-1-methyl-N-(4-(2,2,2-trifluoroacetamido)phenyl)-1H-pyrrole-2-carboxamide hydrochloride (16) (68 mg, 0.19 mmol) was added to a mixture of 4-(((6aS)-5-((allyloxy)carbonyl)-2-methoxy-12-oxo-6-((tetrahydro-2H-pyran-2-yl)oxy)-5,6,6a,7,8,9,10,12-octahydrobenzo[e]pyrido[1,2-a][1,4]diazepin-3-yl)oxy)butanoic acid (14) (11.0 mg, 0.21 mmol), 4-(dimethylamino)pyridine (69 mg, 0.56 mmol) and N-(3-dimethylaminopropyl)-N′-ethylcarbodiimide hydrochloride (90 mg, 0.47 mmol) in N,N-dimethylformamide (4 mL), which was previously stirred for 30 min. The resulting solution was stirred at room temperature for 18 h. The reaction mixture was quenched with a saturated aqueous solution of sodium hydrogen carbonate (5 mL) and diluted with brine (50 mL). The aqueous phase was extracted with ethyl acetate (2×30 mL) and the combined organic extracts were then concentrated in vacuo. The resulting residue was purified by column chromatography (silica), eluting with methanol/dichloromethane (from 0% to 10%), to give the title compound (140 mg, 87%) as a viscous brown oil (mixture of diastereomers).

¹H NMR (600 MHz, MeOD) δ 8.01 (s, 1H), 7.66 (d, J=9.0 Hz, 2H), 7.63-7.60 (m, 2H), 7.16-7.14 (m, 1H), 6.90 (s, 1H), 6.85 (s, 1H), 5.97 (d, J=8.1 Hz, 1H), 5.80 (s, 1H), 5.09 (d, J=11.3 Hz, 2H), 4.31-4.19 (m, 2H), 4.15-4.02 (m, 4H), 3.91-3.81 (m, 9H), 3.52 (dt, J=10.2, 4.1 Hz, 1H), 2.56 (t, J=7.2 Hz, 2H), 2.21-2.16 (m, 2H), 1.81-1.53 (m, 12H); ¹³C NMR (150 MHz, MeOD) δ 172.6, 171.5, 162.3, 157.0, 156.6, 152.0, 150.7, 137.9, 133.7, 133.5, 129.4, 126.7, 124.5, 123.2, 122.7, 122.4, 118.5, 118.0, 116.6, 115.8, 111.7, 106.8, 101.4, 83.6, 69.6, 67.6, 63.3, 58.0, 56.8, 54.9, 50.0, 40.1, 37.0, 31.7, 26.6, 24.1, 20.6, 19.3, 18.5; MS (ES+): m/z=842 (M+H)⁺; LCMS (Method B): t_{R}=4.00 min.

### Allyl (6aS)-3-(4-((5-((4-aminophenyl)carbamoyl)-1-methyl-1H-pyrrol-3-yl)amino)-4-oxobutoxy)-2-methoxy-12-oxo-6-((tetrahydro-2H-pyran-2-yl)oxy)-6,6a,7,8,9,10-hexahydrobenzo[e]pyrido[1,2-a][1,4]diazepine-5(12H)-carboxylate (18)

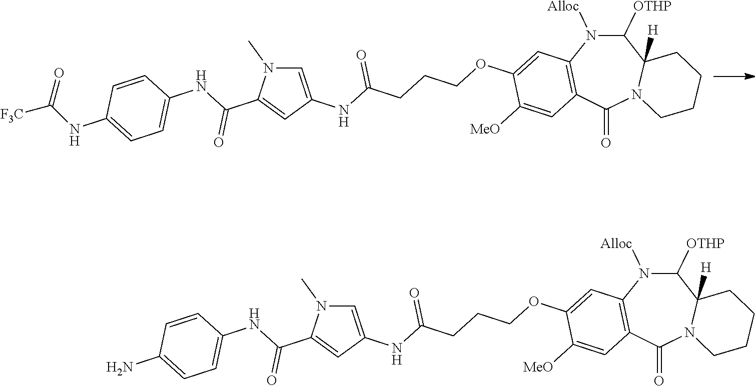

A solution of allyl (6aS)-2-methoxy-3-(4-((1-methyl-5-((4-(2,2,2-trifluoroacetamido)phenyl)carbamoyl)-1H-pyrrol-3-yl)amino)-4-oxobutoxy)-12-oxo-6-((tetrahydro-2H-pyran-2-yl)oxy)-6,6a,7,8,9,10-hexahydrobenzo[e]pyrido[1,2-a][1,4]diazepine-5(12H)-carboxylate (17) (138 mg, 0.164 mmol) in 1,4-dioxane (5 mL) was charged with an aqueous solution of sodium hydroxide (1 M, 5.0 mL, 5.00 mmol). The reaction mixture was stirred at room temperature for 4 h and was then concentrated in vacuo, after which it was diluted into brine (20 mL). The aqueous layer was extracted with ethyl acetate (2×20 mL) and the combined organic extracts were then dried over sodium sulfate, filtered, and concentrated in vacuo, to give the title compound (120 mg, 98%) as a grey solid (mixture of diastereomers).

¹H NMR (600 MHz, DMSO-d₆) δ 9.85 (s, 1H), 9.41 (s, 1H), 7.28 (d, J=8.7 Hz, 2H), 7.17 (s, 1H), 7.06 (s, 1H), 6.90 (s, 1H), 6.81 (s, 1H), 6.51 (d, J=8.7 Hz, 2H), 5.98 (dd, J=57.3, 9.9 Hz, 1H), 5.80-5.69 (m, 1H), 5.18-4.69 (m, 5H), 4.67-4.35 (m, 2H), 4.09-3.93 (m, 3H), 3.82 (s, 3H), 3.79 (d, J=3.0 Hz, 3H), 3.79-3.72 (m, 2H), 3.56-3.45 (m, 1H), 2.90 (s, 1H), 2.42 (t, J=7.1 Hz, 2H), 2.04-1.97 (m, 2H), 1.75-1.32 (m, 12H); ¹³C NMR (150 MHz, DMSO-d₆) δ 169.3, 168.6, 162.8, 159.8, 150.3, 149.2, 145.2, 133.2, 128.7, 127.7, 126.3, 123.6, 122.6, 122.4, 118.5, 116.8, 114.5, 114.1, 110.9, 104.4, 94.7, 83.9, 68.5, 66.1, 56.2, 55.3, 38.7, 36.5, 36.2, 32.1, 31.2, 30.6, 25.4, 25.2, 24.9, 23.1, 23.0, 19.4, 18.3; MS (ES+): m/z=745 (M+H)⁺; LCMS (Method A): t_{R}=5.88 min.

### (S)—N-(4-Aminophenyl)-4-(4-((2-methoxy-12-oxo-6a,7,8,9,10,12-hexahydrobenzo[e]pyrido[1,2-a][1,4]diazepin-3-yl)oxy)butanamido)-1-methyl-1H-pyrrol-2-carboxamide (19)

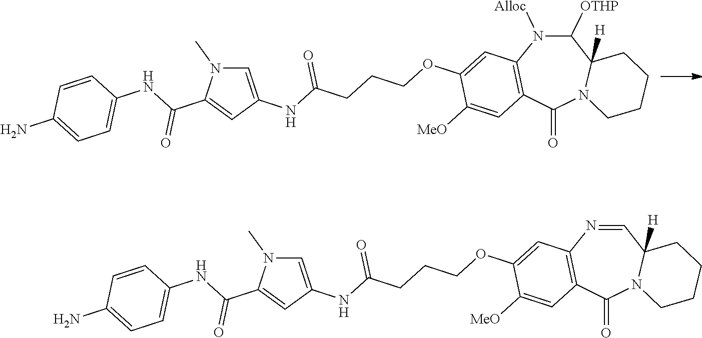

A solution of allyl (6aS)-3-(4-((5-((4-aminophenyl)carbamoyl)-1-methyl-1H-pyrrol-3-yl)amino)-4-oxobutoxy)-2-methoxy-12-oxo-6-((tetrahydro-2H-pyran-2-yl)oxy)-6,6a, 7,8,9,10-hexahydrobenzo[e]pyrido[1,2-a][1,4]diazepine-5(12H)-carboxylate (18) (43 mg, 0.05 mmol) in dichloromethane (2 mL) was charged with tetrakis(triphenylphosphine)palladium(0) (3 mg, 5 mol %), and pyrrolidine (5 μL, 0.06 mmol). The resulting mixture was stirred at room temperature for 30 min and then concentrated in vacuo and subjected to strong vacuum for 40 min. Purification by flash column chromatography (silica) eluting with methanol/dichloromethane (from 0% to 10%) gave the title compound (21.8 mg, 78%) as cream solid.

[α]_{D}²⁰=93.0° (c 1.36, DMSO); ¹H NMR (600 MHz, DMSO-d₆) δ 9.85 (s, 1H), 9.43 (s, 1H), 8.00 (d, J=5.7 Hz, 1H), 7.31-7.26 (m, 3H), 7.17 (d, J=1.8 Hz, 1H), 6.83 (d, J=1.8 Hz, 1H), 6.80 (s, 1H), 6.51 (d, J=8.8 Hz, 2H), 4.84 (s, 2H), 4.14-4.05 (m, 2H), 3.82 (s, 3H), 3.79 (s, 3H), 3.74-3.64 (m, 3H), 2.43 (t, J=7.4 Hz, 2H), 2.04 (dt, J=13.9, 6.8 Hz, 2H), 1.90-1.83 (m, 1H), 1.76-1.55 (m, 5H); ¹³C NMR (150 MHz, DMSO-d₆) δ 168.8, 166.3, 164.7, 159.3, 150.2, 147.1, 144.7, 139.9, 128.2, 123.2, 122.1, 121.9, 120.7, 118.1, 113.7, 111.4, 109.5, 104.0, 67.8, 55.6, 54.9, 48.6, 39.5, 36.01, 31.9, 24.7, 23.7, 22.6, 17.7; MS (ES+): m/z=559 (M+H)⁺; LCMS (Method B): t_{R}=2.53 min; HRMS (ESI, m/z): calc, for C₃₀H₃₅N₆O₅⁺ ([M]+H)⁺ 559.2663 found 559.2651.

### N-(4-((S)-2-((S)-2-(6-(2,5-Dioxo-2,5-dihydro-1H-pyrrol-1-yl) hexanamido)-3-methylbutanamido)propanamido)phenyl)-4-(4-(((S)-2-methoxy-12-oxo-6a,7,8,9,10,12-hexahydrobenzo[e]pyrido[1,2-a][1,4]diazepin-3-yl)oxy)butanamido)-1-methyl-1H-pyrrole-2-carboxamide (20)

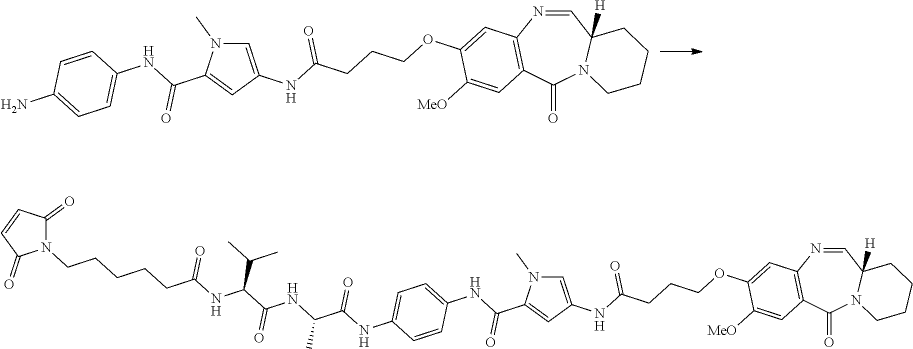 [SMILES: COc1cc2c(cc1OCCCC(=O)Nc1cc(C(=O)Nc3ccc(N)cc3)n(C)c1)N=C[C@@H]1CCCCN1C2=O ; COc1cc2c(cc1OCCCC(=O)Nc1cc(C(=O)Nc3ccc(NC(=O)[C@H](C)NC(=O)[C@@H](NC(=O)CCCCCN4C(=O)C=CC4=O)C(C)C)cc3)n(C)c1)N=C[C@@H]1CCCCN1C2=O]

A solution of (S)—N-(4-aminophenyl)-4-(4-((2-methoxy-12-oxo-6a,7,8,9,10,12-hexahydrobenzo[e]pyrido[1,2-a][1,4]diazepin-3-yl)oxy)butanamido)-1-methyl-1H-pyrrol-2-carboxamide (19) (350 mg, 0.63 mmol) and (6-(2,5-dioxo-2,5-dihydro-1H-pyrrol-1-yl)hexanoyl)-L-valyl-L-alanine (252 mg, 0.95 mmol) in dichloromethane/methanol (19:1, 20 mL) was cooled to 0° C. and charged with ethyl 1,2-dihydro-2-ethoxyquinoline-1-carboxylate (234 mg, 0.95 mmol). The resulting mixture was stirred for 18 h at room temperature, whereupon it was diluted into petroleum spirit, 40-60° C. (300 mL) and filtered under reduced pressure. Preparative TLC (silica), eluting with methanol/dichloromethane (5%) gave the title compound (158 mg, 27%) as a yellow solid.

¹H NMR (400 MHz, DMSO-d₆) δ 9.91 (s, 1H), 9.84 (s, 1H), 9.76 (s, 1H), 8.13 (d, J=6.8 Hz, 1H), 8.00 (d, J=5.6 Hz, 1H), 7.82 (d, J=8.6 Hz, 1H), 7.65-7.58 (m, 2H), 7.57-7.46 (m, 2H), 7.27 (s, 1H), 7.21 (s, 1H), 6.99 (s, 2H), 6.97-6.91 (d, J=1.9 Hz, 1H), 6.80 (s, 1H), 4.37 (t, J=7.2 Hz, 1H), 4.20-4.08 (m, 3H), 4.07-3.89 (m, 2H), 3.87-3.64 (m, 7H), 3.17 (d, J=5.2 Hz, 2H), 2.44 (t, J=7.4 Hz, 2H), 2.24-2.10 (m, 2H), 2.05 (h, J=7.6, 7.2 Hz, 3H), 1.96 (q, J=6.8 Hz, 1H), 1.89-1.41 (m, 10H), 1.30 (d, J=7.0 Hz, 3H), 1.23 (s, 1H), 0.84 (dd, J=16.0, 6.8 Hz, 6H); MS (ES+): m/z=922 (M+H)⁺; LCMS (Method F): t_{R}=3.02 min.

### Methyl 4-(4-((tert-butoxycarbonyl)amino)phenyl)-1-methyl-1H-pyrrol-2-carboxylate (21)

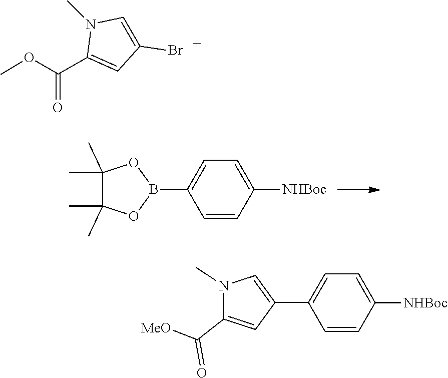

A solution of methyl 4-bromo-1-methyl-1H-pyrrole-2-carboxylate (500 mg, 2.3 mmol), in acetonitrile (10 mL) and water (5 mL), was charged with tert-butyl (4-(4,4,5,5-tetramethyl-1,3,2-dioxaborolan-2-yl)phenyl)carbamate (805 mg, 2.52 mmol), potassium carbonate (954 mg, 6.9 mmol), and tetrakis(triphenylphosphine)palladium (0) (140 mg, 5 mol %) and the resulting mixture was flushed with argon and then irradiated with microwaves at 100° C. for 2 min. The mixture was then filtered through a pad of celite, which was washed with ethyl acetate and the resulting organic filtrate was concentrated in vacuo. Purification by flash column chromatography (silica), eluting with ethyl acetate/petroleum spirit, 40-60° C. (from 0% to 40%), gave the title compound (475 mg, 63%) as a white solid.

¹H NMR (600 MHz, DMSO-d₆) δ 9.29 (s, 1H), 7.50 (d, J=2.0 Hz, 1H), 7.46-7.39 (m, 4H), 7.15 (d, J=2.1 Hz, 1H), 3.87 (s, 3H), 3.76 (s, 3H), 1.47 (s, 9H); ¹³C NMR (150 MHz, DMSO-d₆) δ 160.8, 152.7, 137.5, 128.0, 126.8, 124.8, 122.8, 122.2, 118.4, 113.7, 78.9, 50.9, 36.5, 28.1; MS (ES+): m/z=331 (M+H)⁺; LCMS (Method B): t_{R}=4.22 min.

### Methyl 4-(4-(4-((tert-butoxycarbonyl)amino)-1-methyl-1H-pyrrol-2-carboxamido)phenyl)-1-methyl-1H-pyrrol-2-carboxylate (22)

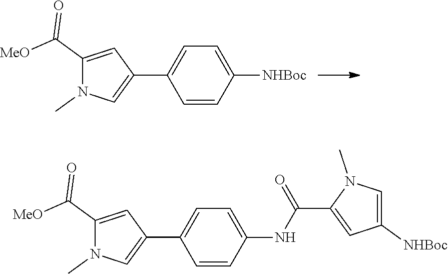 [SMILES: COC(=O)c1cc(-c2ccc(NC(=O)OC(C)(C)C)cc2)cn1C ; COC(=O)c1cc(-c2ccc(NC(=O)c3cc(NC(=O)OC(C)(C)C)cn3C)cc2)cn1C]

A solution of methyl 4-(4-((tert-butoxycarbonyl)amino)phenyl)-1-methyl-1H-pyrrol-2-carboxylate (21) (950 mg, 2.88 mmol) in 1,4-dioxane (4 mL) and methanol (4 mL), was charged with hydrogen chloride (4 M in 1,4-dioxane, 8 mL), dropwise. The resulting mixture was stirred for 3 h and then concentrated in vacuo. The residue was then added to a mixture of 4-((tert-butoxycarbonyl)amino)-1-methyl-1H-pyrrol-2-carboxylic acid (830 mg, 3.45 mmol), 4-(dimethylamino)pyridine (1.05 g, 8.64 mmol) and N-(3-dimethylaminopropyl)-N′-ethylcarbodiimide hydrochloride (1.38 g, 7.20 mmol) in N,N-dimethylformamide (15 mL), which was previously stirred for 30 min. The resulting mixture was then stirred at room temperature for 18 h, before diluting into a saturated aqueous solution of sodium hydrogen carbonate (10 mL) and brine (150 mL). This was then extracted with ethyl acetate (2×80 mL) and the combined organic extracts were then concentrated in vacuo. Purification by flash column chromatography (silica), eluting with acetone/petroleum spirit, 40-60° C. (from 0% to 40%), gave the title compound (860 mg, 66%) as a cream solid.

¹H NMR (600 MHz, DMSO-d₆) δ 9.76 (s, 1H), 9.11 (s, 1H), 7.69 (d, J=8.7 Hz, 2H), 7.55 (d, J=2.0 Hz, 1H), 7.50 (d, J=8.7 Hz, 2H), 7.19 (d, J=2.1 Hz, 1H), 6.93 (d, J=6.2 Hz, 2H), 3.89 (s, 3H), 3.81 (s, 3H), 3.77 (s, 3H), 1.46 (s, 9H); ¹³C NMR (150 MHz, DMSO-d₆) δ 160.8, 159.6, 152.9, 137.4, 128.8, 127.0, 124.6, 122.8, 122.4, 122.2, 120.4, 117.7, 113.8, 104.6, 78.3, 51.0, 36.5, 36.1, 28.2; MS (ES+): m/z=453 (M+H)⁺; LCMS (Method B): t_{R}=4.07 min.

### Allyl (6aS)-2-methoxy-3-(4-((5-((4-(5-(methoxycarbonyl)-1-methyl-1H-pyrrol-3-yl)phenyl)carbamoyl)-1-methyl-1H-pyrrol-3-yl)amino)-4-oxobutoxy)-12-oxo-6-((tetrahydro-2H-pyran-2-yl)oxy)-6,6a,7,8,9,10-hexahydrobenzo[e]pyrido[1,2-a][1,4]diazepin-5(12H)-carboxylate (23)

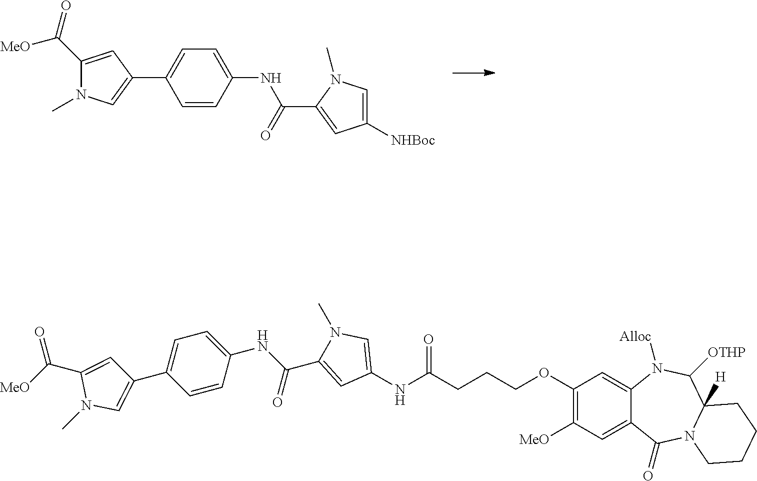

A solution of methyl 4-(4-(4-((tert-butoxycarbonyl)amino)-1-methyl-1H-pyrrol-2-carboxamido)phenyl)-1-methyl-1H-pyrrol-2-carboxylate (22) (250 mg, 0.552 mmol) in 1,4-dioxane (1 mL) and methanol (1 mL), was charged with hydrogen chloride (4 M in 1,4-dioxane, 3 mL), dropwise. The resulting mixture was stirred for 4 h and then concentrated in vacuo. The residue was added to a mixture of 4-(((6aS)-5-((allyloxy)carbonyl)-2-methoxy-12-oxo-6-((tetrahydro-2H-pyran-2-yl)oxy)-5,6,6a,7,8,9,10,12-octahydrobenzo[e]pyrido[1,2-a][1,4]diazepin-3-yl)oxy)butanoic acid (14) (272 mg, 0.511 mmol), 4-(dimethylamino)pyridine (208 mg, 1.70 mmol) and N-(3-dimethylaminopropyl)-N′-ethylcarbodiimide hydrochloride (272 mg, 1.42 mmol) in N,N-dimethylformamide (5 mL), which was previously stirred for 30 min. The resulting solution was stirred at room temperature for 18 h, before being diluted into ethyl acetate (60 mL), and washed with brine (50 mL). The organic phase was concentrated in vacuo. Purification by flash column chromatography (silica), eluting with acetone/dichloromethane (from 0% to 30%), gave the title compound (242 mg, 51%) as a brown solid.

¹H NMR (600 MHz, DMSO-d₆) δ 9.89 (s, 1H), 9.78 (s, 1H), 7.68 (d, J=8.8 Hz, 2H), 7.55 (d, J=1.9 Hz, 1H), 7.51 (d, J=8.7 Hz, 2H), 7.22 (d, J=1.7 Hz, 1H), 7.20 (d, J=2.0 Hz, 1H), 7.06 (d, J=4.2 Hz, 1H), 6.94 (d, J=1.8 Hz, 1H), 6.90 (s, 1H), 5.99-5.97 (m, 1H), 5.82-5.66 (m, 1H), 5.26-4.91 (m, 3H), 4.60-4.39 (m, 2H), 4.15-3.93 (m, 4H), 3.89 (s, 3H), 3.84-3.80 (m, 7H), 3.77 (s, 3H), 3.54-3.45 (m, 1H), 2.90 (d, J=10.9 Hz, 1H), 2.43 (t, J=7.1 Hz, 2H), 2.05-2.00 (m, 2H), 1.72-1.38 (m, 12H); ¹³C NMR (150 MHz, DMSO-d₆) δ 168.9, 168.8, 168.1, 168.1, 160.8, 159.6, 155.3, 149.8, 148.7, 137.3, 132.7, 128.9, 127.2, 127.0, 125.8, 124.6, 122.8, 122.7, 122.3, 122.1, 120.4, 118.8, 116.4, 114.0, 113.8, 110.4, 104.7, 94.3, 83.4, 68.1, 65.6, 62.0, 55.7, 54.8, 51.0, 39.5, 38.2, 36.5, 36.1, 31.7, 30.5, 30.2, 24.9, 24.8, 24.5, 22.6, 22.6, 18.9, 17.8; MS (ES+): m/z=867 (M+H)⁺; LCMS (Method B): t_{R}=4.17 min.

### 4-(4-(4-(4-(((6aS)-5-((Allyloxy)carbonyl)-2-methoxy-12-oxo-6-((tetrahydro-2H-pyran-2-yl)oxy)-5,6,6a,7,8,9,10,12-octahydrobenzo[e]pyrido[1,2-a][1,4]diazepin-3-yl)oxy)butanamido)-1-methyl-1H-pyrrol-2-carboxamido)phenyl)-1-methyl-1H-pyrrol-2-carboxylic acid (24)

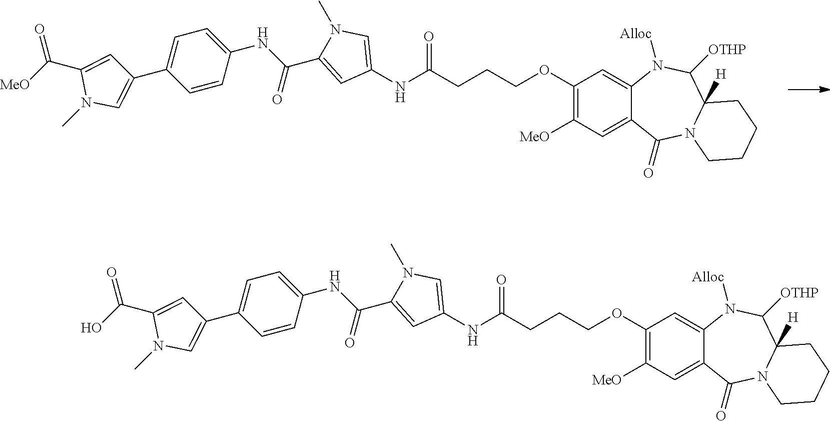

A solution of allyl (6aS)-2-methoxy-3-(4-((5-((4-(5-(methoxycarbonyl)-1-methyl-1H-pyrrol-3-yl)phenyl)carbamoyl)-1-methyl-1H-pyrrol-3-yl)amino)-4-oxobutoxy)-12-oxo-6-((tetrahydro-2H-pyran-2-yl)oxy)-6,6a,7,8,9,10-hexahydrobenzo[e]pyrido[1,2-a][1,4]diazepin-5(12H)-carboxylate (23) (600 mg, 0.69 mmol) in 1,4-dioxane (10 mL) was charged with an aqueous solution of sodium hydroxide (1 M, 10 mL) and the resulting mixture was stirred at room temperature for 18 h, then concentrated in vacuo. Water (100 mL) was then added, and the mixture was acidified to pH=5, with dropwise addition of glacial acetic acid. After extracting with ethyl acetate (2×100 mL), the combined organic extracts were concentrated to give the title compound (558 mg, 97%) as a cream solid (mixture of diastereomers).

¹H NMR (600 MHz, DMSO-d₆) δ 12.26 (s, 1H), 9.89 (s, 1H), 9.78 (s, 1H), 7.68 (d, J=8.1 Hz, 2H), 7.54-7.44 (m, 3H), 7.22 (s, 1H), 7.14 (d, J=2.0 Hz, 1H), 7.06 (d, J=4.3 Hz, 1H), 6.98-6.77 (m, 2H), 6.14 (d, J=9.9 Hz, 1H), 5.83-5.75 (m, 1H), 5.24-4.91 (m, 3H), 4.69-4.35 (m, 2H), 4.18-3.93 (m, 3H), 3.88 (s, 3H), 3.86-3.69 (m, 8H), 3.56-3.44 (m, 1H), 2.89 (dd, J=23.0, 10.8 Hz, 1H), 2.44 (t, J=6.9 Hz, 2H), 2.04 (dp, J=20.8, 6.9 Hz, 2H), 1.76-1.29 (m, 12H); ¹³C NMR (150 MHz, DMSO-d₆) δ 169.3, 168.6, 168.5, 162.4, 160.1, 155.3, 149.8, 149.2, 149.0, 137.7, 133.2, 129.7, 127.7, 126.9, 125.0, 123.8, 123.2, 122.9, 122.6, 120.9, 119.2, 117.0, 114.5, 114.2, 110.8, 105.2, 99.7, 94.7, 88.0, 83.9, 68.5, 68.3, 66.0, 63.2, 62.5, 56.2, 55.4, 38.7, 37.0, 36.6, 32.2, 30.6, 25.4, 25.2, 23.1, 19.6, 18.3; MS (ES+): m/z=853 (M+H)⁺; LCMS (Method B): t_{R}=3.83 min.

### Allyl (6aS)-3-(4-((5-((4-(5-((4-aminophenyl)carbamoyl)-1-methyl-1H-pyrrol-3-yl)phenyl)carbamoyl)-1-methyl-1H-pyrrol-3-yl)amino)-4-oxobutoxy)-2-methoxy-12-oxo-6-((tetrahydro-2H-pyran-2-yl)oxy)-6,6a,7,8,9,10-hexahydrobenzo[e]pyrido[1,2-a][1,4]diazepin-5(12H)-carboxylate (25)

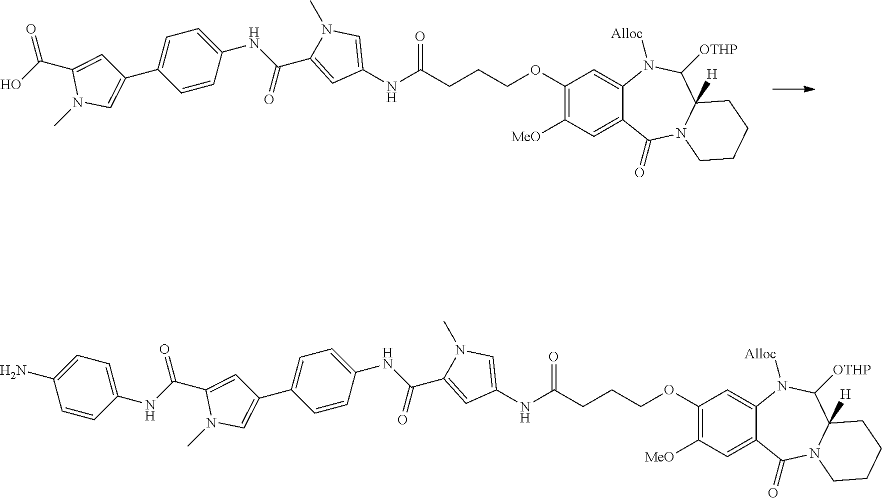

A solution of 4-(4-(4-(4-(((6aS)-5-((allyloxy)carbonyl)-2-methoxy-12-oxo-6-((tetrahydro-2H-pyran-2-yl)oxy)-5,6,6a,7,8,9,10,12-octahydrobenzo[e]pyrido[1,2-a][1,4]diazepin-3-yl)oxy)butanamido)-1-methyl-1H-pyrrol-2-carboxamido)phenyl)-1-methyl-1H-pyrrol-2-carboxylic acid (24) (210 mg, 0.246 mmol) in N,N-dimethylformamide (8 mL) was charged with 4-(dimethylamino)pyridine (90 mg, 0.74 mmol) and N-(3-dimethylaminopropyl)-N′-ethylcarbodiimide hydrochloride (118 mg, 0.615 mmol), and stirred for 30 min at room temperature. To the resulting mixture, 1,4-benzenediamine (40 mg, 0.370 mmol) was added and stirred for a further 18 h. The reaction mixture was then diluted into a saturated aqueous solution of sodium hydrogen carbonate (100 mL) and extracted with ethyl acetate (2×40 mL). The combined organic extracts were concentrated in vacuo. Purification by flash column chromatography (silica), eluting with methanol/dichloromethane (from 0% to 20%), gave the title compound (110 mg, 47%) as a cream solid (mixture of diastereomers).

¹H NMR (600 MHz, DMSO-d₆) δ 9.89 (s, 1H), 9.78 (s, 1H), 9.48 (s, 1H), 7.70 (d, J=8.6 Hz, 2H), 7.48 (d, J=8.5 Hz, 2H), 7.39 (d, J=1.7 Hz, 1H), 7.36-7.32 (m, 2H), 7.30 (d, J=1.8 Hz, 1H), 7.23-7.20 (m, 1H), 7.06 (d, J=4.1 Hz, 1H), 6.97-6.94 (m, 1H), 6.80 (s, 1H), 6.57-6.52 (m, 2H), 6.03 (d, J=10.0 Hz, 1H), 5.81-5.72 (m, 1H), 5.26-4.95 (m, 3H), 4.92 (s, 2H), 4.64-4.36 (m, 2H), 4.14-3.95 (m, 3H), 3.89 (s, 3H), 3.83 (dd, J=6.0, 2.4 Hz, 6H), 3.81-3.70 (m, 1H), 3.55-3.34 (m, 2H), 3.00-2.82 (m, 1H), 2.44 (t, J=7.0 Hz, 2H), 2.09-1.99 (m, 2H), 1.94-1.35 (m, 12H); ¹³C NMR (150 MHz, DMSO-d₆) δ 169.4, 168.6, 160.1, 159.7, 149.2, 145.1, 137.5, 133.2, 128.8, 127.1, 125.1, 124.8, 123.2, 122.6, 122.4, 122.3, 121.0, 119.2, 117.0, 114.3, 110.9, 110.8, 110.1, 105.2, 99.7, 94.7, 88.0, 83.9, 68.5, 68.3, 66.0, 63.2, 56.2, 55.4, 38.7, 38.6, 36.8, 36.6, 32.2, 32.1, 30.9, 30.6, 25.4, 25.2, 24.9, 23.1, 23.0, 19.6, 19.4, 18.3; MS (ES+): m/z=943 (M+H)⁺; LCMS (Method B): t_{R}=3.45 min.

### (S)—N-(4-aminophenyl)-4-(4-(4-(4-((2-methoxy-12-oxo-6a,7,8,9,10,12-hexahydrobenzo[e]pyrido[1,2-a][1,4]diazepin-3-yl)oxy)butanamido)-1-methyl-1H-pyrrol-2-carboxamido)phenyl)-1-methyl-1H-pyrrol-2-carboxamide (26)

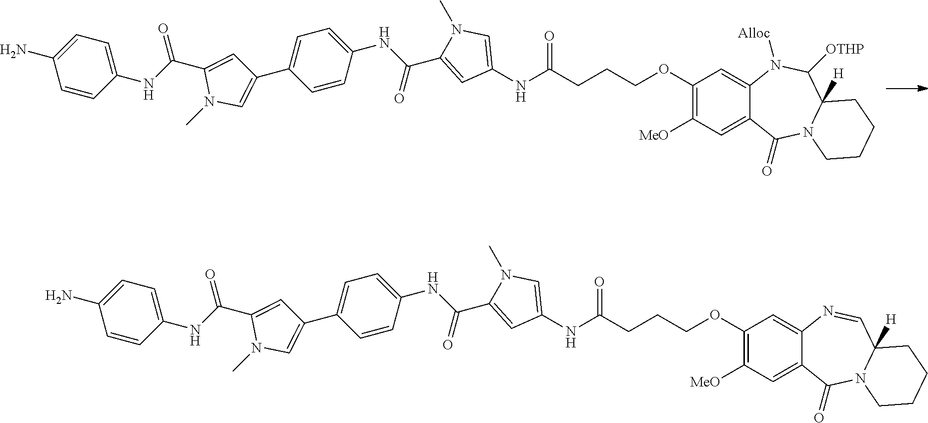

A solution of allyl (6aS)-3-(4-((5-((4-(5-((4-aminophenyl)carbamoyl)-1-methyl-1H-pyrrol-3-yl)phenyl)carbamoyl)-1-methyl-1H-pyrrol-3-yl)amino)-4-oxobutoxy)-2-methoxy-12-oxo-6-((tetrahydro-2H-pyran-2-yl)oxy)-6,6a,7,8,9,10-hexahydrobenzo[e]pyrido[1,2-a][1,4]diazepin-5(12H)-carboxylate (25) (250 mg, 0.265 mmol) in dichloromethane (3 mL) was charged with tetrakis(triphenylphosphine) palladium (0) (15 mg, 5 mol %), and pyrrolidine (26 μL, 0.32 mmol) and the resulting mixture was stirred at room temperature for 20 min, before being subjected to strong vacuum for 30 min. The resulting residue was then purified by flash column chromatography (silica), eluting with methanol/dichloromethane (from 0% to 10%) to give the title compound (118 mg, 59%) as a cream solid.

[α]_{D}²³=85° (c 0.143, DMSO); ¹H NMR (600 MHz, DMSO-d₆) δ 9.90 (s, 1H), 9.78 (s, 1H), 9.48 (s, 1H), 8.00 (d, J=5.6 Hz, 1H), 7.70 (d, J=8.6 Hz, 2H), 7.48 (d, J=8.6 Hz, 2H), 7.39 (d, J=1.5 Hz, 1H), 7.33 (d, J=8.7 Hz, 2H), 7.30 (d, J=1.5 Hz, 1H), 7.27 (s, 1H), 7.21 (d, J=1.4 Hz, 1H), 6.96 (d, J=1.5 Hz, 1H), 6.80 (s, 1H), 6.53 (d, J=8.7 Hz, 2H), 4.86 (s, 2H), 4.13 (dt, J=9.6, 6.3 Hz, 1H), 4.07-3.97 (m, 2H), 3.88 (s, 3H), 3.83 (d, J=5.1 Hz, 6H), 3.72 (dt, J=8.4, 4.1 Hz, 1H), 3.16-3.08 (m, 1H), 2.44 (t, J=7.4 Hz, 2H), 2.07-2.02 (m, 3H), 1.89-1.59 (m, 5H); ¹³C NMR (150 MHz, DMSO-d₆) δ 168.9, 166.3, 164.7, 159.6, 159.2, 150.2, 147.2, 144.8, 139.9, 137.0, 129.7, 128.2, 126.7, 124.7, 124.4, 122.7, 122.1, 121.9, 121.8, 120.7, 120.5, 118.8, 113.7, 111.4, 109.6, 109.5, 104.8, 67.8, 55.6, 49.3, 36.3, 36.2, 31.9, 24.7, 23.7, 22.6, 17.7; MS (ES+): m/z=757 (M+H)⁺; LCMS (Method A): t_{R}=5.80 min; HRMS (EI, m/z): calculated for C₄₂H₄₅N₈O₆⁺ (M+H)⁺757.3457 found 757.3457.

### N-(4-((S)-2-((S)-2-(6-(2,5-Dioxo-2,5-dihydro-1H-pyrrol-1-yl) hexanamido)-3-methylbutanamido)propanamido)phenyl)-4-(4-(4-(4-(((S)-2-methoxy-12-oxo-6a,7,8,9,10,12-hexahydrobenzo[e]pyrido[1,2-a][1,4]diazepin-3-yl)oxy)butanamido)-1-methyl-1H-pyrrol-2-carboxamido)phenyl)-1-methyl-1H-pyrrol-2-carboxamide (27)

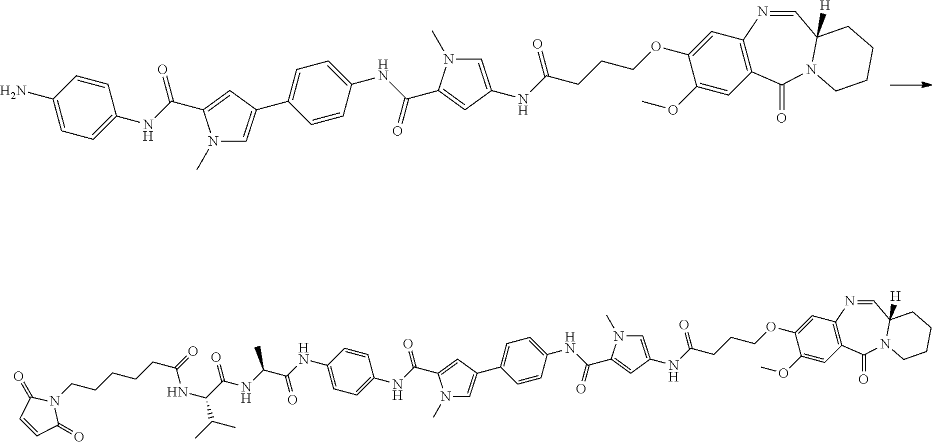 [SMILES: COc1cc2c(cc1OCCCC(=O)Nc1cc(C(=O)Nc3ccc(-c4cc(C(=O)Nc5ccc(N)cc5)n(C)c4)cc3)n(C)c1)N=C[C@@H]1CCCCN1C2=O ; COc1cc2c(cc1OCCCC(=O)Nc1cc(C(=O)Nc3ccc(-c4cc(C(=O)Nc5ccc(NC(=O)[C@H](C)NC(=O)[C@@H](NC(=O)CCCCCN6C(=O)C=CC6=O)C(C)C)cc5)n(C)c4)cc3)n(C)c1)N=C[C@@H]1CCCCN1C2=O]

A solution of (S)—N-(4-aminophenyl)-4-(4-(4-(4-((2-methoxy-12-oxo-6a, 7,8,9,10,12-hexahydrobenzo[e]pyrido[1,2-a][1,4]diazepin-3-yl)oxy)butanamido)-1-methyl-1H-pyrrol-2-carboxamido)phenyl)-1-methyl-1H-pyrrol-2-carboxamide (26) (200 mg, 0.264 mmol), in dichloromethane (3 mL) and methanol (300 μL) was cooled to 0° C., then charged with 2-ethoxy-1-ethoxycarbonyl-1,2-dihydroquinoline (98 mg, 0.396 mmol) and (6-(2,5-dioxo-2,5-dihydro-1H-pyrrol-1-yl)hexanoyl)-L-valyl-L-alanine (106 mg, 0.277 mmol). The resulting mixture was stirred at room temperature for 16 h, and then concentrated in vacuo. Purification by flash column chromatography (silica), eluting with methanol/dichloromethane (from 0% to 10%) gave the title compound (150 mg, 51%) as a white solid.

[α]_{D}²²=85° (c 0.200, DMSO); ¹H NMR (600 MHz, DMSO-d₆) δ 9.90 (s, 1H), 9.84 (s, 1H), 9.79 (s, 2H), 8.12 (d, J=7.0 Hz, 1H), 8.00 (d, J=5.7 Hz, 1H), 7.81 (d, J=8.6 Hz, 1H), 7.71 (d, J=8.7 Hz, 2H), 7.65 (d, J=9.0 Hz, 2H), 7.54 (d, J=9.0 Hz, 2H), 7.49 (d, J=8.6 Hz, 2H), 7.44 (d, J=1.5 Hz, 1H), 7.38 (d, J=1.7 Hz, 1H), 7.27 (s, 1H), 7.22 (t, J=1.7 Hz, 1H), 6.99 (s, 2H), 6.97 (d, J=1.6 Hz, 1H), 6.80 (s, 1H), 4.42-4.35 (m, 1H), 4.17 (dd, J=8.6, 6.9 Hz, 1H), 4.14-3.93 (m, 4H), 3.91 (s, 3H), 3.83 (d, J=5.7 Hz, 6H), 3.74-3.65 (m, 3H), 3.37 (t, J=7.1 Hz, 2H), 3.15-3.08 (m, 1H), 2.47-2.42 (m, 2H), 2.21-1.93 (m, 4H), 1.90-1.73 (m, 2H), 1.60-1.44 (m, 4H), 1.31 (d, J=7.1 Hz, 3H), 1.22-1.14 (m, 4H), 0.85 (dd, J=23.3, 6.8 Hz, 6H); ¹³C NMR (150 MHz, DMSO-d₆) δ 172.3, 171.0, 171.0, 170.7, 168.9, 166.3, 164.7, 159.6, 159.5, 150.7, 150.2, 147.1, 139.9, 137.1, 134.8, 134.4, 129.5, 126.2, 125.3, 124.4, 122.7, 122.1, 121.9, 120.7, 120.5, 120.3, 119.3, 111.4, 110.3, 109.5, 104.8, 67.8, 57.5, 55.9, 55.6, 49.3, 48.9, 39.5, 37.0, 36.5, 36.2, 34.9, 31.9, 30.4, 27.7, 25.8, 24.9, 23.7, 22.6, 19.2, 18.2, 18.0, 17.7; MS (ES+): m/z=1121 (M+H)⁺; LCMS (Method B): t_{R}=3.57 min; HRMS (ESI, m/z): calc, for C₆₀H₇₀N₁₁O₁₁⁺ (M+H)⁺1120.5250 found 1120.5257.

### tert-Butyl ((2S)-1-(((2S)-1-(((1S,9S)-9-ethyl-5-fluoro-9-hydroxy-4-methyl-10,13-dioxo-2,3,9,10,12,13,13a,14-octahydro-1H-benzo[de]pyrano[3′,4′:5,6]indeno[1,2-b]quinolin-1-yl)amino)-1-oxopropan-2-yl)amino)-3-methyl-1-oxobutan-2-yl)carbamate (28)

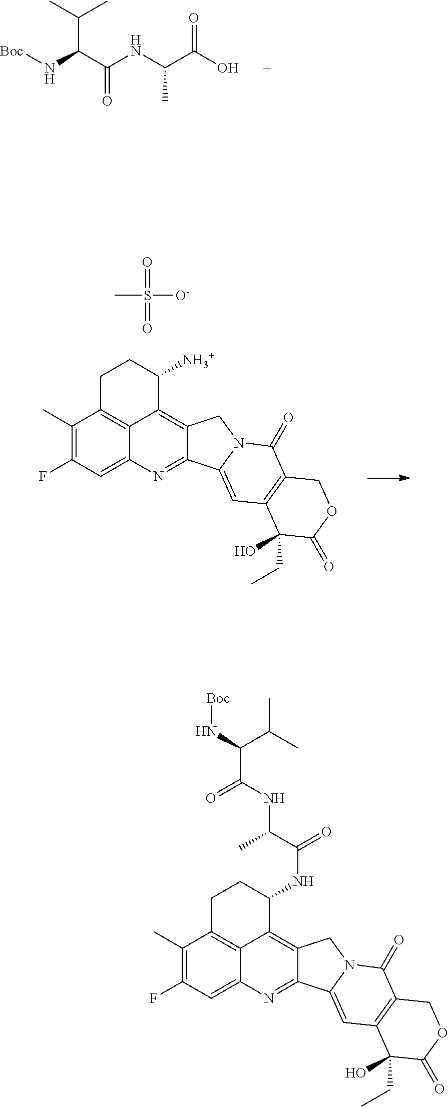

A solution of (tert-butoxycarbonyl)-L-valyl-L-alanine (54.7 mg, 0.19 mmol), exatecan mesylate (100 mg, 0.19 mmol) and N,N-diisopropylethylamine (100 μL, 0.57 mmol) in N,N-dimethylformamide (3 mL) was charged with HATU (80 mg, 0.19 mmol) and the resulting mixture was stirred for 10 min at room temperature, whereupon it was concentrated in vacuo, to give the title compound, which was used in the subsequent step without any further purification.

### (2S)-2-Amino-N-((2S)-1-(((1S,9S)-9-ethyl-5-fluoro-9-hydroxy-4-methyl-10,13-dioxo-2,3,9,10,12,13,13a,14-octahydro-1H-benzo[de]pyrano[3′,4′:5,6]indeno[1,2-b]quinolin-1-yl)amino)-1-oxopropan-2-yl)-3-methylbutanamide trifluoroacetate (29)

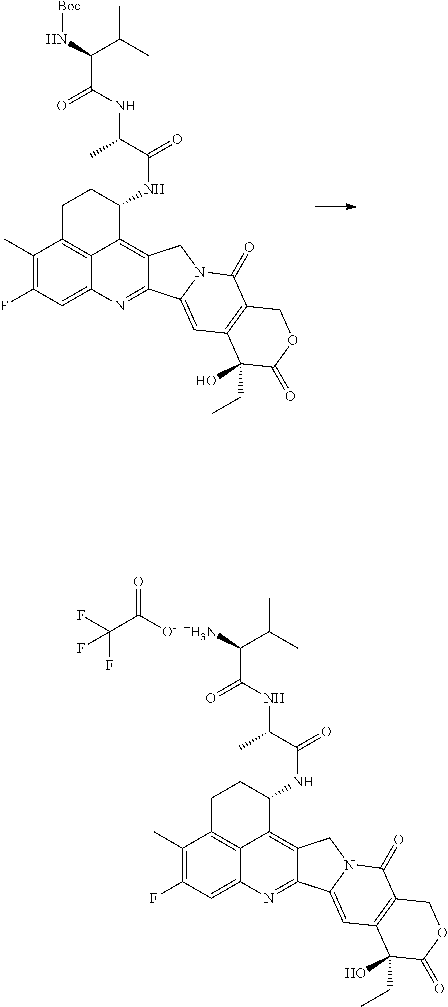 [SMILES: CC[C@@]1(O)C(=O)OCc2c1cc1n(c2=O)Cc2c-1nc1cc(F)c(C)c3c1c2[C@@H](NC(=O)[C@H](C)NC(=O)[C@@H](NC(=O)OC(C)(C)C)C(C)C)CC3 ; O=C([O-])C(F)(F)F ; CC[C@@]1(O)C(=O)OCc2c1cc1n(c2=O)Cc2c-1nc1cc(F)c(C)c3c1c2[C@@H](NC(=O)[C@H](C)NC(=O)[C@@H]([NH3+])C(C)C)CC3]

tert-Butyl ((2S)-1-(((2S)-1-(((1S,9 S)-9-ethyl-5-fluoro-9-hydroxy-4-methyl-10,13-dioxo-2,3,9,10,12,13,13a,14-octahydro-1H-benzo[de]pyrano[3′,4′:5,6]indeno[1,2-b]quinolin-1-yl)amino)-1-oxopropan-2-yl)amino)-3-methyl-1-oxobutan-2-yl)carbamate (28) (134 mg, crude) was solubilised into trifluoroacetic acid (1.5 mL) and dichloromethane (3 mL) and the resulting mixture was stirred for 15 min, whereupon it was purified by reverse-phase preparative HPLC (0.1% trifluoroacetic acid in water/acetonitrile) and lyophilised to give the title compound (96 mg, 71%, two steps).

### 1-(3-(2,5-Dioxo-2,5-dihydro-1H-pyrrol-1-yl)propanamido)-N-((2S)-1-(((2S)-1-(((1S,9S)-9-ethyl-5-fluoro-9-hydroxy-4-methyl-10,13-dioxo-2,3,9,10,12,13,13a,14-octahydro-1H-benzo[de]pyrano[3′,4′:5,6]indeno[1,2-b]quinolin-1-yl)amino)-1-oxopropan-2-yl)amino)-3-methyl-1-oxobutan-2-yl)-3,6,9,12,15,18,21,24-octaoxaheptacosan-27-amide (30)

A solution of 1-(2,5-dioxo-2,5-dihydro-1H-pyrrol-1-yl)-3-oxo-7,10,13,16,19,22,25,28-octaoxa-4-azahentriacontan-31-oic acid (46 mg, 0.078 mmol) in N,N-dimethylformamide (2 mL) was charged with N,N-diisopropylethylamine (47 μL, 0.27 mmol) and HATU (30 mg, 0.079 mmol). After stirring for 1 min at room temperature, (2S)-2-amino-N-((2S)-1-(((1S,9S)-9-ethyl-5-fluoro-9-hydroxy-4-methyl-10,13-dioxo-2,3,9,10,12,13,13a,14-octahydro-1H-benzo[de]pyrano[3′,4′:5,6]indeno[1,2-b]quinolin-1-yl)amino)-1-oxopropan-2-yl)-3-methylbutanamide trifluoroacetate (29) (56 mg, 0.078 mmol) was added and stirring was continued for another 10 min. The resulting mixture was purified by reverse-phase preparative HPLC (0.1% trifluoroacetic acid in water/acetonitrile) and the pure fractions lyophilised to give the title compound (60 mg, 65%) as a yellow solid.

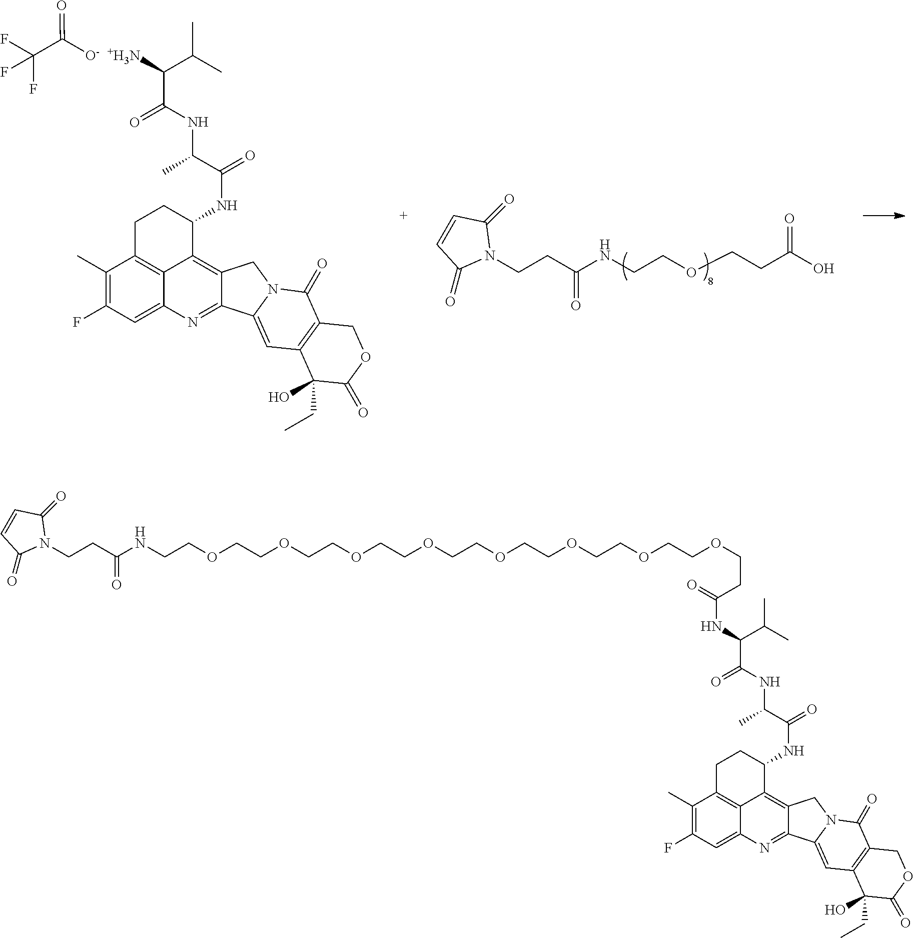

¹H NMR (400 MHz, CDCl₃) 7.61-7.60 (m, 2H), 7.42 (d, J=8.0 Hz, 1H), 7.04 (d, J=8.0 Hz, 1H), 6.94 (d, J=8.0 Hz, 1H), 6.69 (s, 2H), 6.48 (br s, 1H), 5.74 (d, J=12.0 Hz, 1H), 5.56-5.55 (m, 1H), 5.47 (d, J=16.0 Hz, 1H), 5.28 (d, J=12.0 Hz, 1H), 5.04 (d, J=16.0 Hz, 1H), 4.57-4.54 (m, 1H), 3.96 (t, J=4.0 Hz, 1H), 3.83-3.80 (m, 3H), 3.63-3.59 (m, 29H), 3.52 (t, J=4.0 Hz, 2H), 3.40 (q, J=4.0 Hz, 2H), 3.26-3.22 (m, 1H), 3.11-3.05 (m, 1H), 2.56-2.52 (m, 3H), 2.40-2.33 (m, 6H), 2.28-2.13 (m, 3H), 1.93-1.84 (m, 2H), 1.47 (d, J=8.0 Hz, 3H), 1.03 (t, J=6.0 Hz, 3H), 0.91 (t, J=6.0 Hz, 6H); MS (ES+): m/z=1180.5 (M+H)⁺; LCMS (5 min): t_{R}=1.87 min; HPLC (15 min): 8.91 min (99.1% purity, 220 nm).

### (9H-Fluoren-9-yl)methyl ((2S)-1-(((2S)-1-((4-(((((1S,9S)-9-ethyl-5-fluoro-9-hydroxy-4-methyl-10,13-dioxo-2,3,9,10,12,13,13a,14-octahydro-1H-benzo[de]pyrano[3′,4′:5,6]indeno[1,2-b]quinolin-1-yl)carbamoyl)oxy)methyl)phenyl)amino)-1-oxopropan-2-yl)amino)-3-methyl-1-oxobutan-2-yl)carbamate (31)

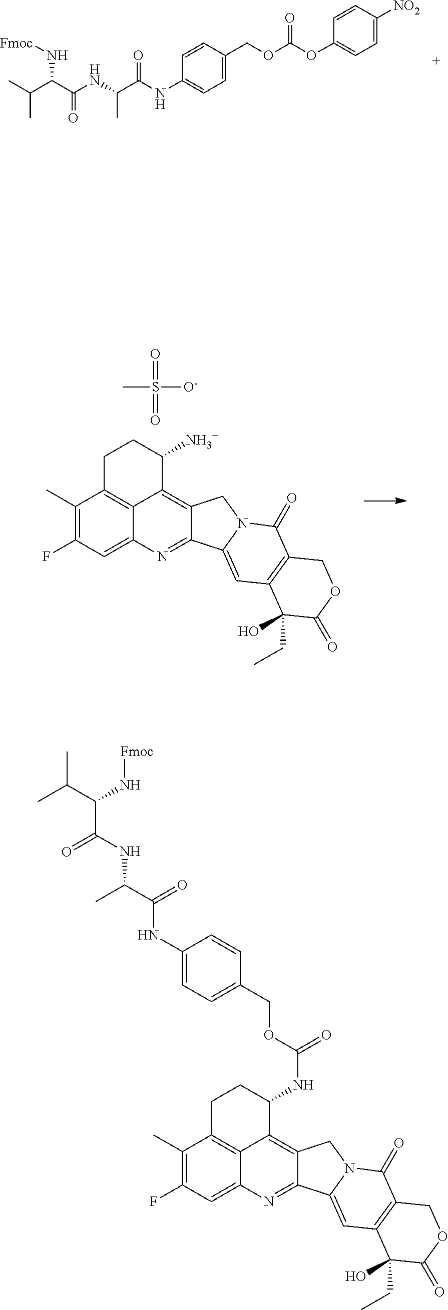

A solution of (9H-fluoren-9-yl)methyl ((S)-3-methyl-1-(((S)-1-((4-((((4-nitrophenoxy)carbonyl)oxy)methyl)phenyl)amino)-1-oxopropan-2-yl)amino)-1-oxobutan-2-yl)carbamate (141 mg, 0.21 mmol) in N,N-dimethylformamide (3 mL) was charged with exatecan mesylate (100 mg, 0.19 mmol), 1-hydroxy-7-azabenzotriazole (10 mg, 0.073 mmol) and N,N-diisopropylethylamine (83 μL, 0.48 mmol), and then stirred at room temperature for 6 h. The resulting mixture was employed in the subsequent step without any further manipulations.

### 4-((S)-2-((S)-2-Amino-3-methylbutanamido)propanamido)benzyl ((1S,9S)-9-ethyl-5-fluoro-9-hydroxy-4-methyl-10,13-dioxo-2,3,9,10,12,13,13a,14-octahydro-1H-benzo[de]pyrano[3′,4′:5,6]indeno[1,2-b]quinolin-1-yl)carbamate trifluoroacetate (32)

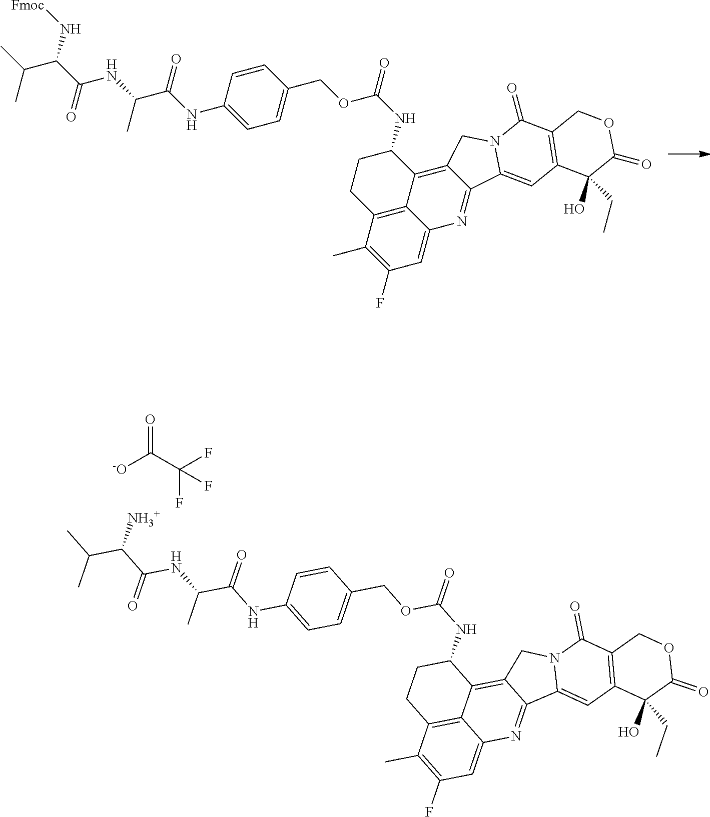

The reaction mixture containing (9H-fluoren-9-yl)methyl ((2S)-1-(((2S)-1-((4-(((((1S,9S)-9-ethyl-5-fluoro-9-hydroxy-4-methyl-10,13-dioxo-2,3,9,10,12,13,13a,14-octahydro-1H-benzo[de]pyrano[3′,4′:5,6]indeno[1,2-b]quinolin-1-yl)carbamoyl)oxy)methyl)phenyl)amino)-1-oxopropan-2-yl)amino)-3-methyl-1-oxobutan-2-yl)carbamate (31) (184 mg, crude) in N,N-dimethylformamide (3 mL) was charged with 1,8-diazabicyclo[5.4.0]undec-7-ene (0.18 mL, 1.20 mmol) dropwise, and then stirred for 10 min, before it was purified by reverse-phase preparative HPLC (0.1% trifluoroacetic acid in water/acetonitrile) and lyophilised to give the title compound (89 mg, 54%, two steps).

### 4-((2S,5S)-37-(2,5-Dioxo-2,5-dihydro-1H-pyrrol-1-yl)-5-isopropyl-2-methyl-4,7,35-trioxo-10,13,16,19,22,25,28,31-octaoxa-3,6,34-triazaheptatriacontanamido)benzyl ((1S,9S)-9-ethyl-5-fluoro-9-hydroxy-4-methyl-10,13-dioxo-2,3,9,10,12,13,13a,14-octahydro-1H-benzo[de]pyrano[3′,4′:5,6]indeno[1,2-b]quinolin-1-yl)carbamate (33)

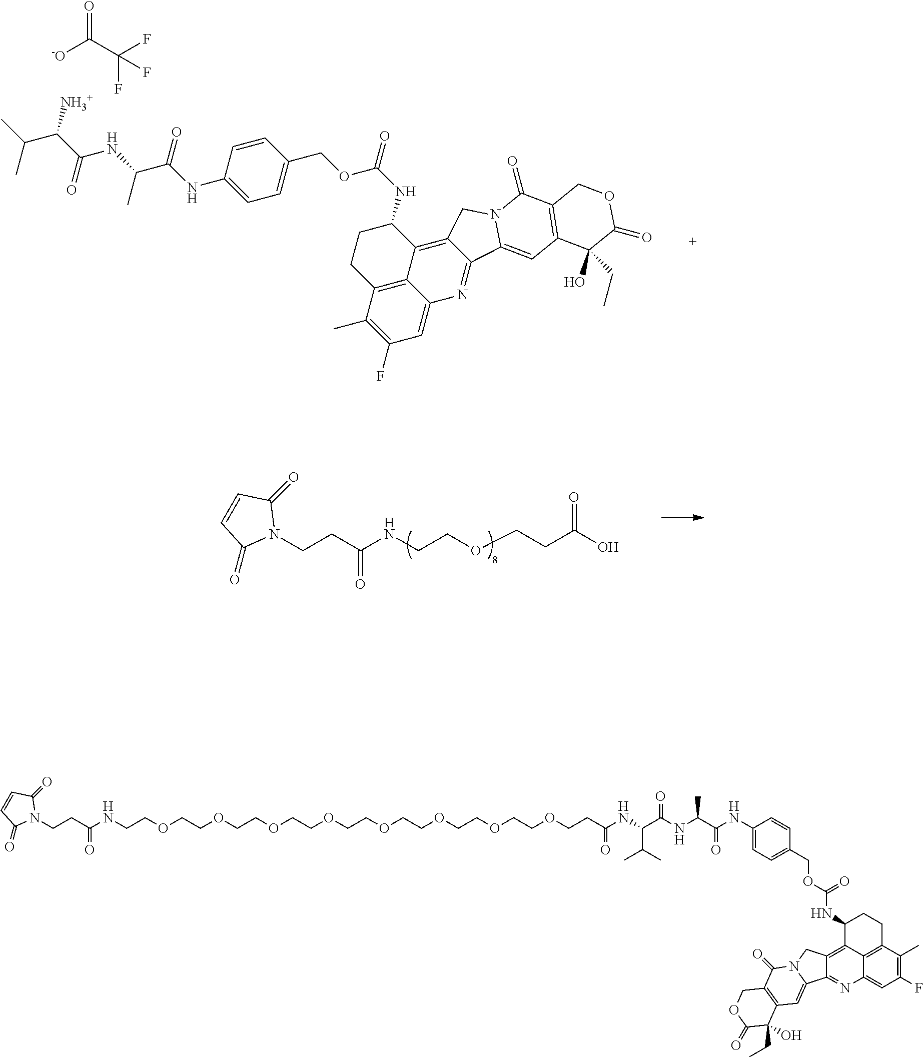

A solution of 1-(2,5-dioxo-2,5-dihydro-1H-pyrrol-1-yl)-3-oxo-7,10,13,16,19,22,25,28-octaoxa-4-azahentriacontan-31-oic acid (37 mg, 0.062 mmol) in N,N-dimethylformamide (2 mL) was charged with N,N-diisopropylethylamine (0.039 mL, 0.22 mmol) and HATU (24 mg, 0.063 mmol), and the resulting mixture was stirred for 1 min at room temperature, before adding 4-((S)-2-((S)-2-amino-3-methylbutanamido)propanamido)benzyl ((1S,9S)-9-ethyl-5-fluoro-9-hydroxy-4-methyl-10,13-dioxo-2,3,9,10,12,13,13a, 14-octahydro-1H-benzo[de]pyrano[3′,4′:5,6]indeno[1,2-b]quinolin-1-yl)carbamate trifluoroacetate (32) (54 mg, 0.062 mmol) and stirring for a further 10 min. Purification by reverse-phase preparative HPLC (0.1% trifluoroacetic acid in water/acetonitrile) followed by lyophilisation, gave the title compound (57 mg, 69%) as a yellow solid.

¹H NMR (400 MHz, CDCl₃) 8.61 (s, 1H), 7.63-7.54 (m, 4H), 7.30 (s, 2H), 7.12 (s, 1H), 7.02 (s, 1H), 6.67 (s, 2H), 6.45 (s, 1H), 5.69 (d, J=12.0 Hz, 1H), 5.50-5.31 (m, 4H), 5.20 (d, J=8.0 Hz, 1H), 5.08 (d, J=8.0 Hz, 1H), 4.62 (br s, 1H), 4.23 (br s, 1H), 3.84-3.70 (m, 4H), 3.67-3.59 (m, 28H), 3.49 (t, J=4.0 Hz, 2H), 3.37 (q, J=4.0 Hz, 2H), 3.16 (br s, 2H), 2.68-2.65 (m, 1H), 2.49-2.46 (m, 3H), 2.41 (s, 3H), 2.35-2.27 (m, 3H), 1.90 (br s, 5H), 1.42 (d, J=8.0 Hz, 3H), 1.04-1.00 (m, 9H); MS (ES+): m/z=1329.8 (M+H)⁺; LCMS (5 min): t_{R}=2.05 min; HPLC (15 min): 10.48 min (100% purity, 220 nm).

### 4-((78S,81S,84S,89S,92S)-78-((2,5,8,11,14,17,20,23,26,29,32,35,38,41,44,47,50,53,56,59,62,65,68,71-Tetracosaoxatriheptacontan-73-yl)carbamoyl)-81-(3-(((S)-1-(((S)-1-((4-((((((S)-4,11-diethyl-9-hydroxy-3,14-dioxo-3,4,12,14-tetrahydro-1H-pyrano[3′,4′:6,7]indolizino[1,2-b]quinolin-4-yl)oxy)carbonyl)oxy)methyl)phenyl)amino)-1-oxopropan-2-yl)amino)-3-methyl-1-oxobutan-2-yl)amino)-3-oxopropyl)-89-isopropyl-92-methyl-75,80,83,87,90-pentaoxo-84-(4-(3-tosyl-2-(tosylmethyl)propanoyl)benzamido)-2,5,8,11,14,17,20,23,26,29,32,35,38,41,44,47,50,53,56,59,62,65,68,71-tetracosaoxa-74,79,82,88,91-pentaazatrinonacontan-93-amido)benzyl ((S)-4,11-diethyl-9-hydroxy-3,14-dioxo-3,4,12,14-tetrahydro-1H-pyrano[3′,4′:6,7]indolizino[1,2-b]quinolin-4-yl) carbonate (34)

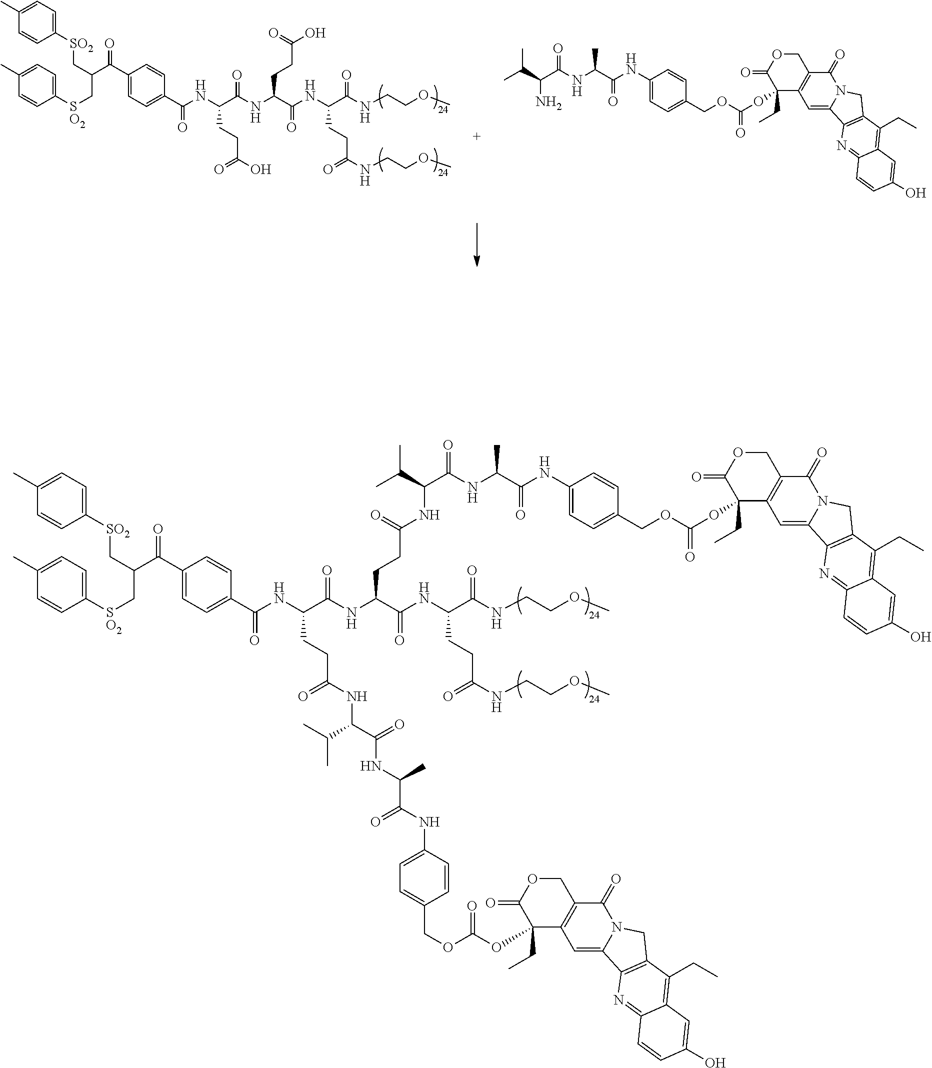

A solution of 4-((S)-2-((S)-2-amino-3-methylbutanamido)propanamido)benzyl ((S)-4,11-diethyl-9-hydroxy-3,14-dioxo-3,4,12,14-tetrahydro-1H-pyrano[3′,4′:6,7]indolizino[1,2-b]quinolin-4-yl) carbonate (41 mg, 0.00576 mmol) and (78S,81S,84S)-78-((2,5,8,11,14,17,20,23,26,29,32,35,38,41,44,47,50,53,56,59,62,65,68,71-tetracosaoxatriheptacontan-73-yl)carbamoyl)-81-(2-carboxyethyl)-75,80,83-trioxo-84-(4-(3-tosyl-2-(tosylmethyl)propanoyl)benzamido)-2,5,8,11,14,17,20,23,26,29,32,35,38,41,44,47,50,53,56,59,62,65,68,71-tetracosaoxa-74,79,82-triazaheptaoctacontan-87-oic acid (80 mg, 0.00264 mmol) in N,N-dimethylformamide (2 mL) was cooled to 0° C. HATU (26 mg, 0.00687 mmol) was then added, followed by NMM (17.5 μL, 0.00158 mmol). The resulting mixture was stirred for 45 min at 0° C., then concentrated in vacuo. Purification by Gilson preparative HPLC (Phenomenex, Luna 5 μm C18(2) 100 Å, LC Column 150×21.2 mm) using gradient method: 14-70% Water-MeCN (0.05% formic acid), followed by lyophilisation, gave the title compound (52.5 mg, 45%) as a yellow solid.

Theoretical exact mass: 4413.1; found: 2207.55 (M+2H)²⁺, 1472.03 (M+3H)³⁺, 1104.28 (M+4H)⁴⁺.

HPLC (14 min): 7.70 min (97.8% purity, 254 nm).

### (9H-Fluoren-9-yl)methyl ((S)-1-(((S)-1-((4-(4-(4-(4-(4-(((S)-2-methoxy-12-oxo-6a,7,8,9,10,12-hexahydrobenzo[e]pyrido[1,2-a][1,4]diazepin-3-yl)oxy)butanamido)-1-methyl-1H-pyrrole-2-carboxamido)phenyl)-1-methyl-1H-pyrrole-2-carboxamido)phenyl)amino)-1-oxopropan-2-yl)amino)-3-methyl-1-oxobutan-2-yl)carbamate (35)

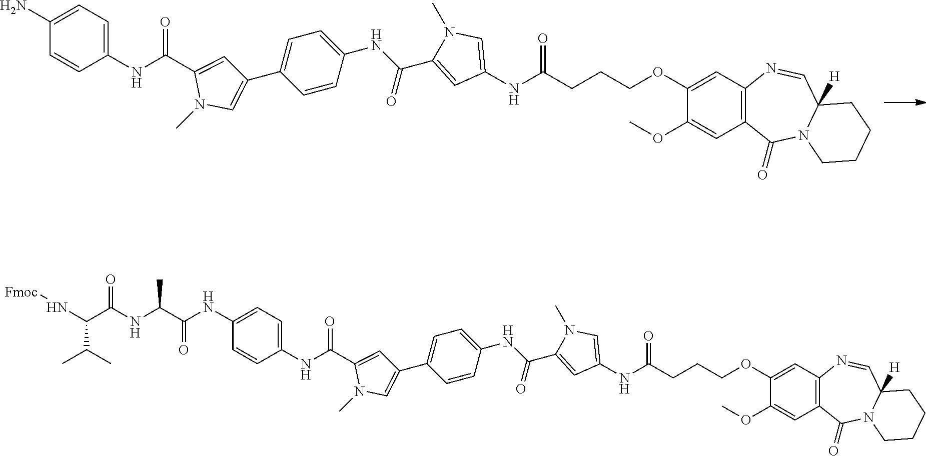

A mixture consisting of (((9H-fluoren-9-yl) methoxy)carbonyl)-L-valyl-L-alanine (325 mg, 0.793 mmol) and ethyl 1,2-dihydro-2-ethoxyquinoline-1-carboxylate (326 mg, 1.32 mmol) in N,N-dimethylformamide (20 mL) was stirred at room temperature for 1 h before adding (S)—N-(4-aminophenyl)-4-(4-(4-(4-((2-methoxy-12-oxo-6a,7,8,9,10,12-hexahydrobenzo[e]pyrido[1,2-a][1,4]diazepin-3-yl)oxy)butanamido)-1-methyl-1H-pyrrol-2-carboxamido)phenyl)-1-methyl-1H-pyrrol-2-carboxamide (26) (500 mg, 0.661 mmol) and stirring the resulting mixture at 0° C. for 5 h, then warming to room temperature and stirring for a further 13 h. After the reaction was judged to be complete by LCMS, the reaction mixture was diluted into dichloromethane/methyl tert-butyl ether (1:8, 200 mL) and stirred for a further 1 h before filtering under reduced pressure. The filter cake was dried under strong vacuum, to give the title compound (500 mg, 66%) as a yellow solid, which was employed in the subsequent step without further purification.

MS (ES+): m/z=1149 (M+H)⁺; LCMS (Method F): t_{R}=3.92 min.

### N-(4-((S)-2-((S)-2-Amino-3-methylbutanamido)propanamido)phenyl)-4-(4-(4-(4-(((S)-2-methoxy-12-oxo-6a,7,8,9,10,12-hexahydrobenzo[e]pyrido[1,2-a][1,4]diazepin-3-yl)oxy)butanamido)-1-methyl-1H-pyrrole-2-carboxamido)phenyl)-1-methyl-1H-pyrrole-2-carboxamide (36)

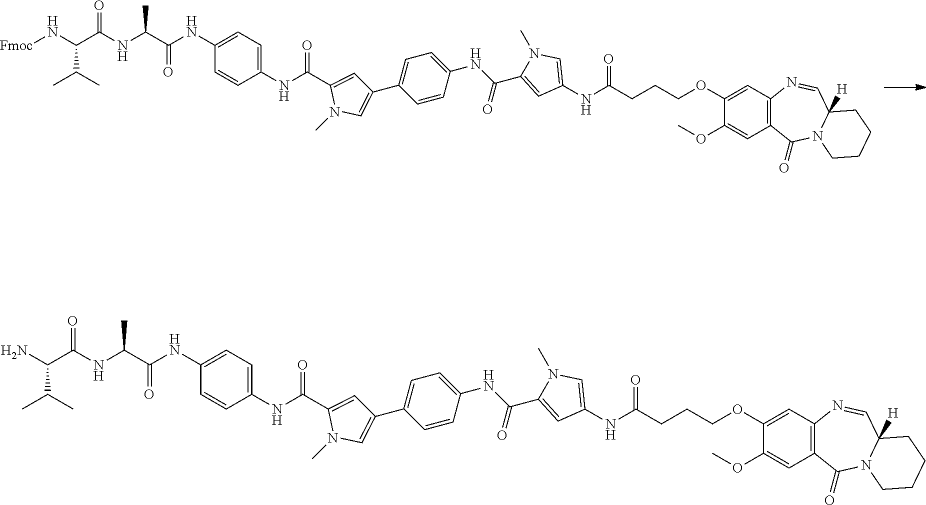

A mixture consisting of (9H-fluoren-9-yl)methyl ((S)-1-(((S)-1-((4-(4-(4-(4-(4-(((S)-2-methoxy-12-oxo-6a,7,8,9,10,12-hexahydrobenzo[e]pyrido[1,2-a][1,4]diazepin-3-yl)oxy)butanamido)-1-methyl-1H-pyrrole-2-carboxamido)phenyl)-1-methyl-1H-pyrrole-2-carboxamido)phenyl)amino)-1-oxopropan-2-yl)amino)-3-methyl-1-oxobutan-2-yl)carbamate (35) (500 mg, 0.435 mmol) and piperidine (111 mg, 1.31 mmol) in N,N-dimethylformamide (10 mL) was stirred at room temperature for 16 h, before LCMS showed consumption of starting material. After dilution into dichloromethane/methyl tert-butyl ether (1:8, 200 mL), the residue was stirred for 1 h before filtration under reduced pressure. The filter cake was then dried under reduced pressure, to give the title compound (400 mg, 99%) as a yellow solid.

MS (ES+): m/z=927 (M+H)⁺; LCMS (Method F): t_{R}=2.81 min.

### (S)—N5-((S)-1-(((S)-1-((4-(4-(4-(4-(4-(((S)-2-Methoxy-12-oxo-6a,7,8,9,10,12-hexahydrobenzo[e]pyrido[1,2-a][1,4]diazepin-3-yl)oxy)butanamido)-1-methyl-1H-pyrrole-2-carboxamido)phenyl)-1-methyl-1H-pyrrole-2-carboxamido)phenyl)amino)-1-oxopropan-2-yl)amino)-3-methyl-1-oxobutan-2-yl)-2-(4-(3-tosyl-2-(tosylmethyl)propanoyl)benzamido)-N1-(2,5,8,11,14,17,20,23,26,29,32,35,38,41,44,47,50,53,56,59,62,65,68,71-tetracosaoxatriheptacontan-73-yl)pentanediamide (37)

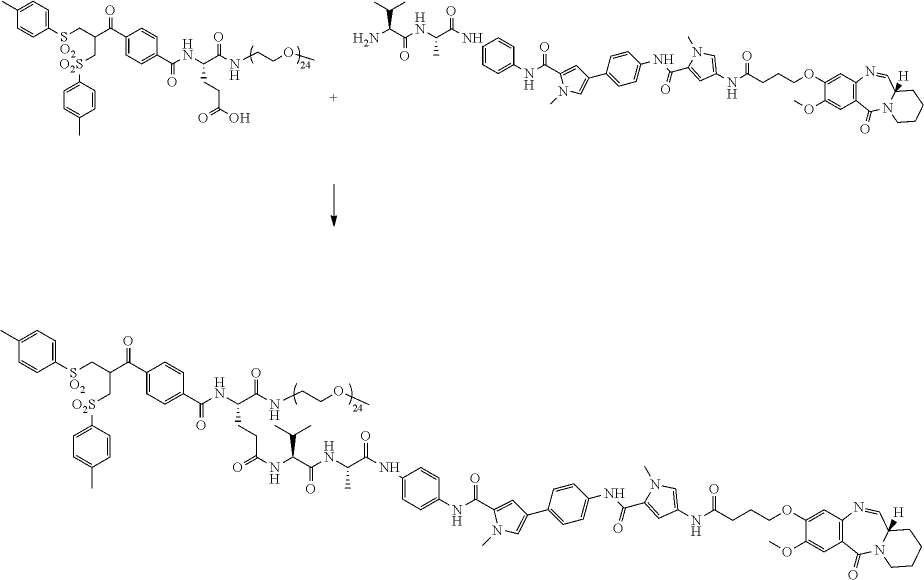

A solution of (S)-75-oxo-76-(4-(3-tosyl-2-(tosylmethyl)propanoyl)benzamido)-2,5,8,11,14,17,20,23,26,29,32,35,38,41,44,47,50,53,56,59,62,65,68,71-tetracosaoxa-74-azanonaheptacontan-79-oic acid (85 mg, 0.05 mmol) and N-(4-((S)-2-((S)-2-amino-3-methylbutanamido)propanamido)phenyl)-4-(4-(4-(4-(((S)-2-methoxy-12-oxo-6a,7,8,9,10,12-hexahydrobenzo[e]pyrido[1,2-a][1,4]diazepin-3-yl)oxy)butanamido)-1-methyl-1H-pyrrole-2-carboxamido)phenyl)-1-methyl-1H-pyrrole-2-carboxamide (36) (51 mg, 0.055 mmol) in N,N-dimethylformamide (2 mL) was cooled to 0° C. and charged with HATU (27 mg, 0.07 mmol) and NMM (16.5 μL, 0.15 mmol), then stirred at this temperature for 1 h. The reaction mixture was purified by reverse-phase chromatography using Gilson preparative HPLC (Phenomenex, Luna 5 μm C18(2) 100 Å, LC Column 150×21.2 mm) using gradient method: 14-70% Water-MeCN (no modifier), followed by lyophilisation, to give the title compound (64 mg, 49%) as a white solid.

Theoretical exact mass: 2607.2; found: 1304.6 (M+2H)²⁺, 870.1 (M+3H)³⁺, 652.8 (M+4H)⁴⁺.

HPLC (14 min): 7.46 min (97.6% purity, 254 nm).

Compound numbers and structure are shown below in Table A.

TABLE A
Compound numbers and structure.

| Compound | |
|---|---|
| No. | Structure |
| 19 | 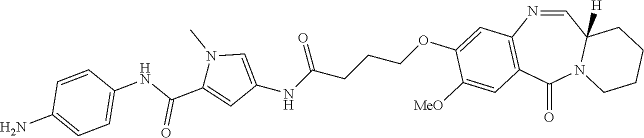 [SMILES: COc1cc2c(cc1OCCCC(=O)Nc1cc(C(=O)Nc3ccc(N)cc3)n(C)c1)N=C[C@@H]1CCCCN1C2=O] |
| 20 | 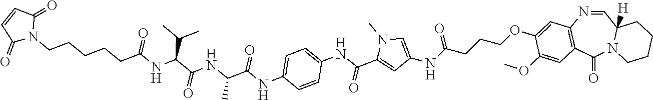 [SMILES: COc1cc2c(cc1OCCCC(=O)Nc1cc(C(=O)Nc3ccc(NC(=O)[C@H](C)NC(=O)[C@@H](NC(=O)CCCCCN4C(=O)C=CC4=O)C(C)C)cc3)n(C)c1)N=C[C@@H]1CCCCN1C2=O] |
| 26 | 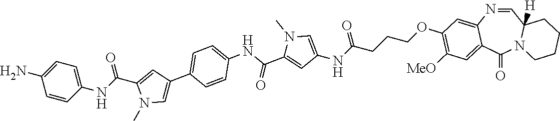 [SMILES: COc1cc2c(cc1OCCCC(=O)Nc1cc(C(=O)Nc3ccc(-c4cc(C(=O)Nc5ccc(N)cc5)n(C)c4)cc3)n(C)c1)N=C[C@@H]1CCCCN1C2=O] |
| 27 | 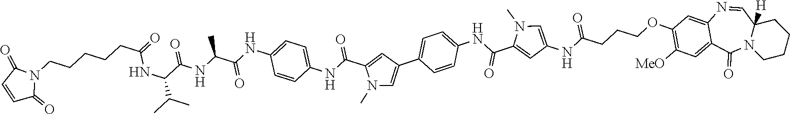 [SMILES: COc1cc2c(cc1OCCCC(=O)Nc1cc(C(=O)Nc3ccc(-c4cc(C(=O)Nc5ccc(NC(=O)[C@H](C)NC(=O)[C@@H](NC(=O)CCCCCN6C(=O)C=CC6=O)C(C)C)cc5)n(C)c4)cc3)n(C)c1)N=C[C@@H]1CCCCN1C2=O] |
| 30 | 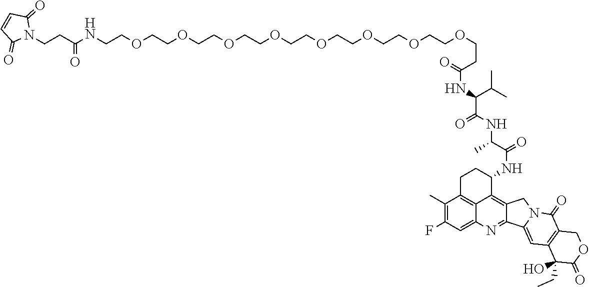 [SMILES: CC[C@@]1(O)C(=O)OCc2c1cc1n(c2=O)Cc2c-1nc1cc(F)c(C)c3c1c2[C@@H](NC(=O)[C@H](C)NC(=O)[C@@H](NC(=O)CCOCCOCCOCCOCCOCCOCCOCCOCCNC(=O)CCN1C(=O)C=CC1=O)C(C)C)CC3] |
| 33 | 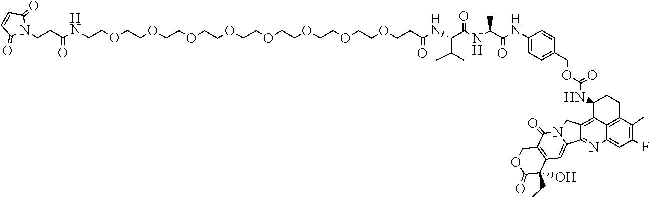 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](C)NC(=O)[C@@H](NC(=O)CCOCCOCCOCCOCCOCCOCCOCCOCCNC(=O)CCN2C(=O)C=CC2=O)C(C)C)cc1)CC3] |
| 34 | 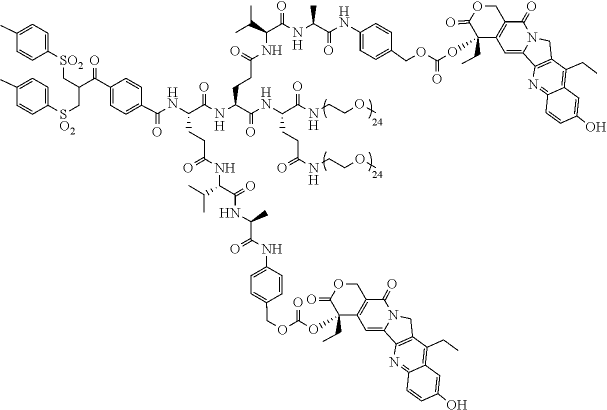 |
| 37 | 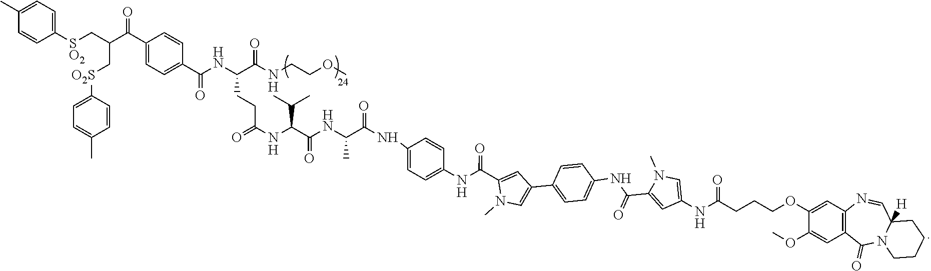 |

### Example 2: Conjugation to CDCP1 mAb and In Vivo Efficacy

### Conjugation of Payload to Antibody

All ADC conjugations were completed using a similar methodology, an example of which is provided below. 21.5 mg IgG1 antibody (8.0 mg/ml in PBS) were charged with EDTA to a final concentration of 2 mM. Reduction was attained by adding 1.27 molar equivalents TCEP (10 mM in water) and incubating for 2 hours at 20° C. After 1.5 hours, a reduction in-process test conjugation with an established payload (e.g., Mal-vcMMAE) was performed, and analyzed by HIC to test for the reduction level. As the target reduction level had not been reached, another 0.1 molar equivalents TCEP were added and the reduction time extended by 1 hour. After 0.5 hours, a second in-process test was run. After confirmation of the desired reduction level, 20% (v/v) Propylene glycol was added to the reduced antibody followed by 6.4 molar equivalents 82 (10 mM stock in DMSO). The solution was incubated for 1 hour at rt. The reaction was quenched by adding 6.4 molar equivalents N-acetylcysteine (10 mM in water). The ADC was buffer exchanged via G25 into PBS and washed by dead-end filtration (Vivaspin-20, 30 kDa MWCO, 0.0006 m²) for 10 DVs. Samples were taken for analysis by HIC, SEC, PLRP, free toxin linker, Endosafe, and the concentration was determined using a SEC calibration curve. Aliquotting was carried out under laminar flow, and the product was stored at −80° C. Only disposable, sterile and pyrogen/DNA/RNA-free plasticware was used.

### Antigen Binding Affinity (FACS)

Test samples were prepared in FACS buffer (PBS, 1% BSA, 0.1% NaN₃) and studies were performed with a 10 μg/mL starting concentration and 3-fold 8-point serial dilution. A427 (Antigen positive) cells were seeded at a density of 1×10⁵ per well of a 96-well assay plate (BD 351177), and samples added at 100 μL per well. Assay plates were incubated on ice for 30 mins.

After 30 minutes, assay plates were centrifuged, supernatant was discarded and cells were washed with FACS buffer. 100 μL of 1:800 Goat pAb Anti-Human IgG (Fcspecific)-PE were added per well. Assay plates were then incubated on ice for a further 30 mins.

At this time point, assay plates were centrifuged, supernatant was discarded and cells were washed with FACS buffer. 100 μL of BD Cellfix (diluted 1:10) in water were added per well. Data readout consisted of fluorescence and was measured using Thermo Fisher Attune NxT Focusing cytometer.

### In Vivo Efficacy

Antitumour activity of the selected ADCs was assessed in tumour xenograft models (both cancer-derived and patient-derived) obtained by inoculation of the relevant cell-line (e.g., MDA-MB-231 in the case of ADC3) in mice.

Maximum tolerated dose (MTD) of the relevant ADC was established on 6-8 CD1 mice (or equivalent) at multiple concentrations on a single dose basis. Once the single dose MTD was determined, an efficacy study was initiated at doses under the maximum tolerated dose.

Briefly, tumours were implanted onto the flank of the mice using a 23-gauge needle, and were randomly assigned to groups (e.g., control or ADC). After implantation, tumours were measured 3 times per week using digital calipers. The length and width of the tumour was measured and volume calculated using the following formula: volume=(length×width²)/2. The bodyweight of all mice on the study was measured and recorded 3 times per week. Mice were observed daily and any signs of distress or changes to general condition (e.g., starred fur, lack of movement, difficulty breathing). Specific criteria were set for early termination, and this only occurred if tumour volume exceeded 1500 mm³, weight loss of ≥15% occurred or animals became compromised (e.g., inability to eat/drink).

Mice were housed in IVC cages (5 mice per cage) with individual mice identified by ear punch. Cages, bedding and water were sanitized before use. Animals were provided with Corn-o-cobs enrichment bedding to provide environment enrichment and nesting material. All animals had free access to a standard certified commercial diet and water. The animal holding room was maintained as follows-room temperature at 20-24° C., humidity at 30-70% and a 12 h light/dark cycle used. Cages were changed once a week with food and water replaced when necessary. All procedures were carried out under the guidelines of the Animal (Scientific Procedures) Act 1986.

### Antibody QC

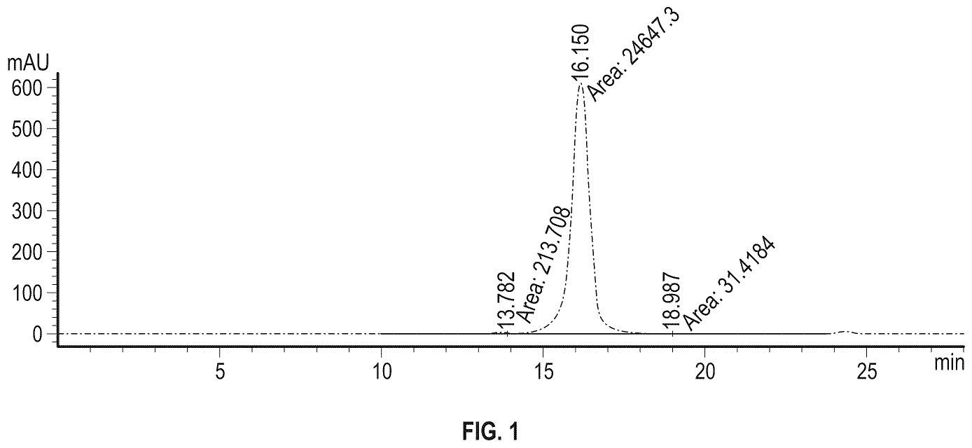

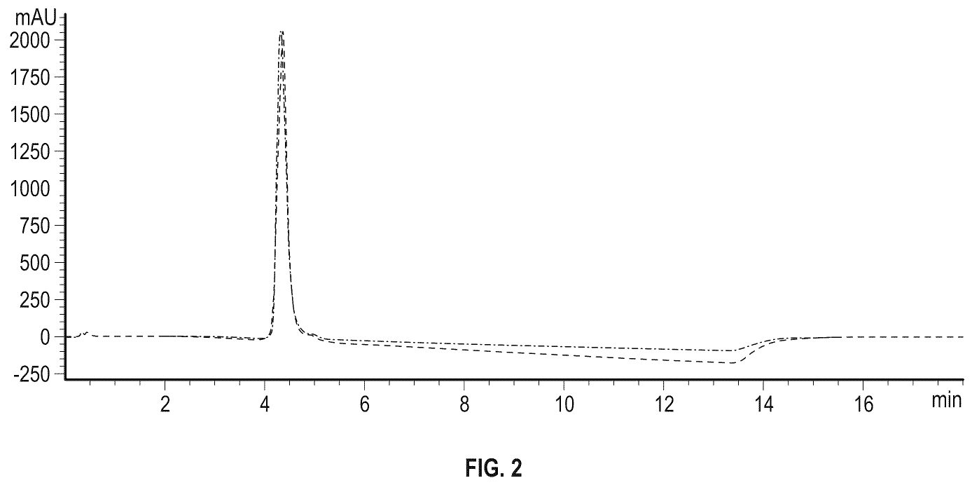

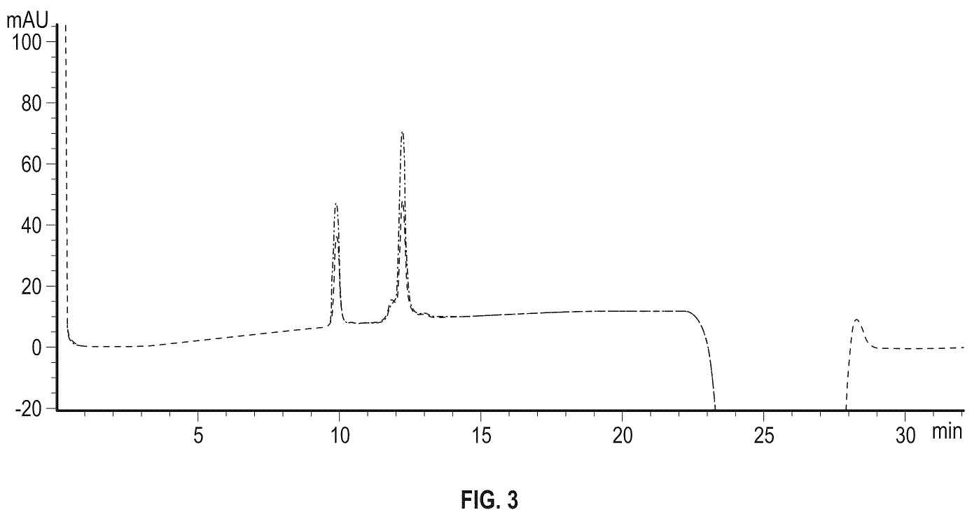

The antibody was of good quality with 99% monomer content via Size Exclusion Chromatography (SEC) (FIG. 1) and HIC (FIG. 2). PLRP showed the expected pattern for reduced Light and Heavy chain. The minor peaks eluting after the main L0 and H0 are likely the result of intrachain disulfide reduction (FIG. 3). The sequences of the light chain variable region, heavy chain variable region, and CDRs (SEQ ID NOs: 1-4 and 5-8) of the antibody can be found in Table 2A (the sequence may also be referred to herein as “Sequence 1”).

### Conjugation of 30 to CDCP1 mAb

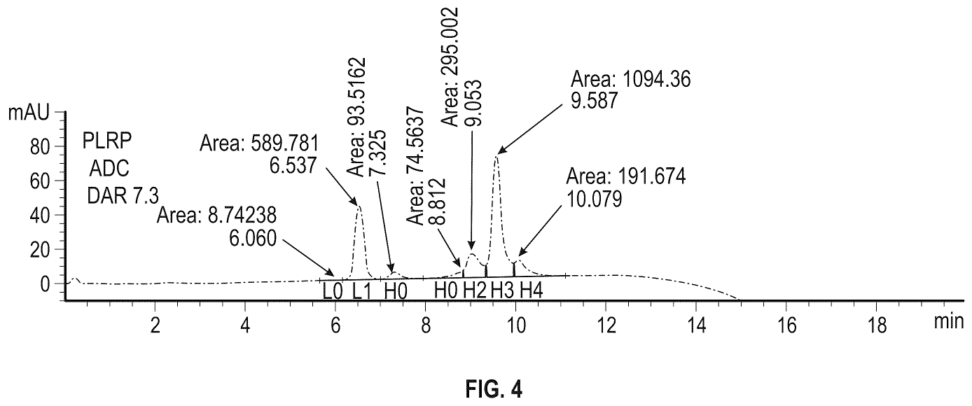

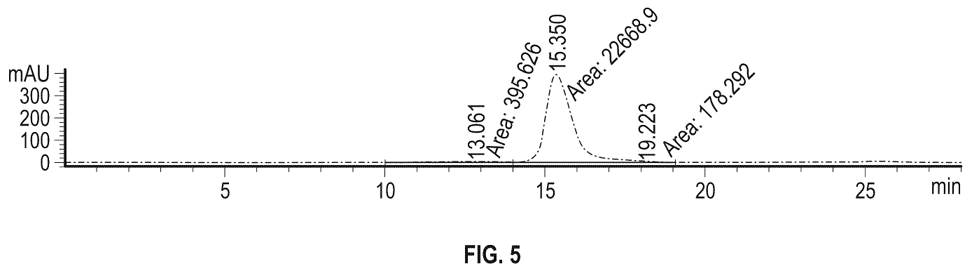

30 was conjugated to an IgG1 antibody targeted to CDCP1 in a stochastic manner to prepare ADC3 forming an ADC of average DAR of 7.3. FIGS. 4 and 5 show PLRP and SEC analysis, respectively. SEC analysis indicated monomeric purity of 97.5%.

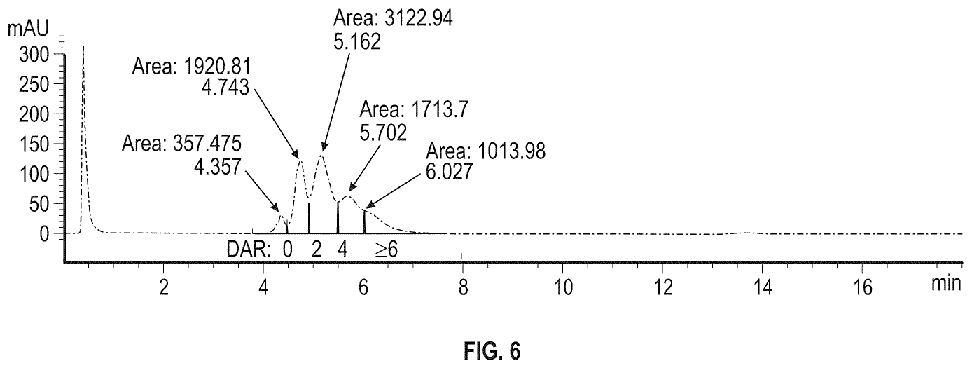

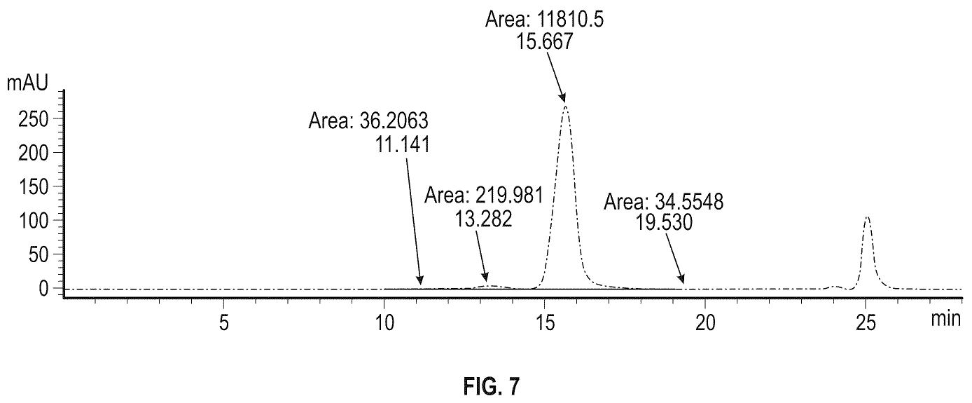

30 was conjugated to an IgG1 antibody targeted to CDCP1 in a stochastic manner to prepare ADC4 forming an ADC of average DAR of 4. FIGS. 6 and 7 show PLRP and SEC analysis, respectively. SEC analysis indicated monomeric purity of 97.6%.

### Conjugation of 33 to CDCP1 mAb

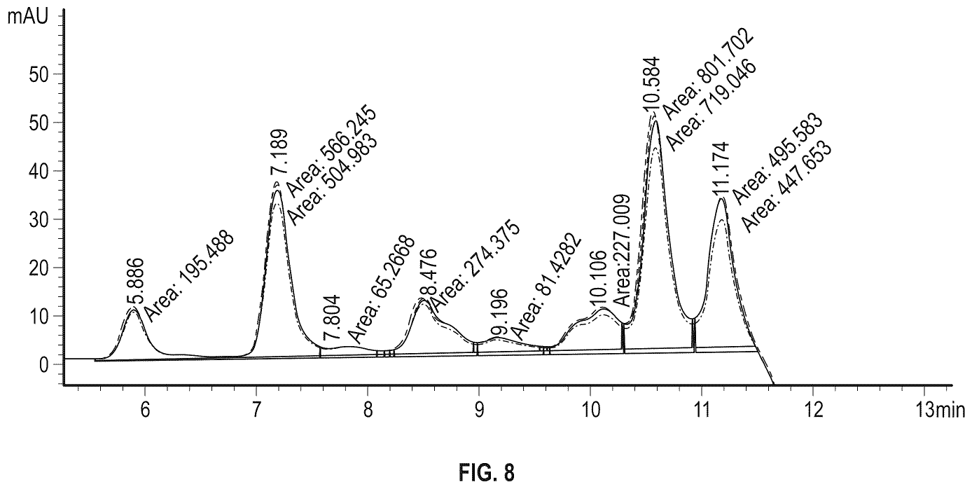

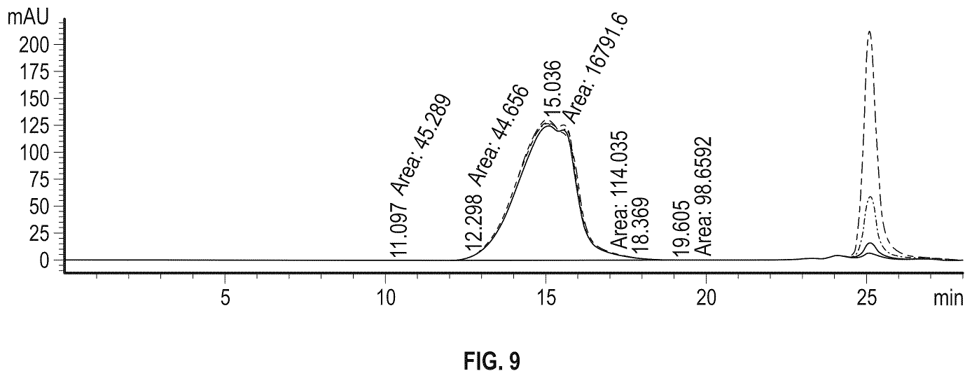

33 was conjugated to an IgG1 antibody targeted to CDCP1 in a stochastic manner to prepare ADC5 forming an ADC of average DAR of 6.7. FIGS. 8 and 9 show PLRP and SEC analysis, respectively.

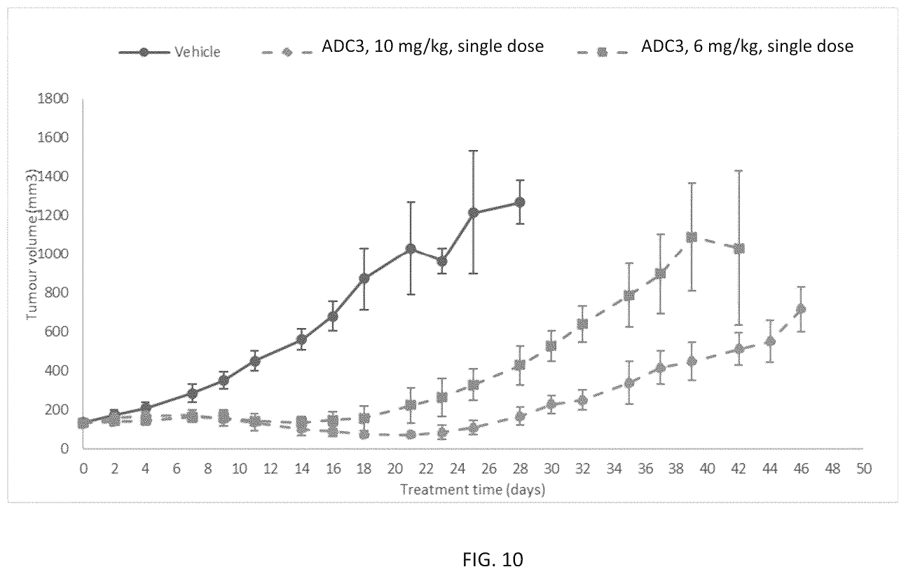

ADC3 was examined in a MDA-MB-231 (Triple Negative Breast Cancer), CDCP1+model at 10 mg/kg and 6 mg/kg single doses and was found to reduce the rate of tumor growth compared to vehicle (FIG. 10).

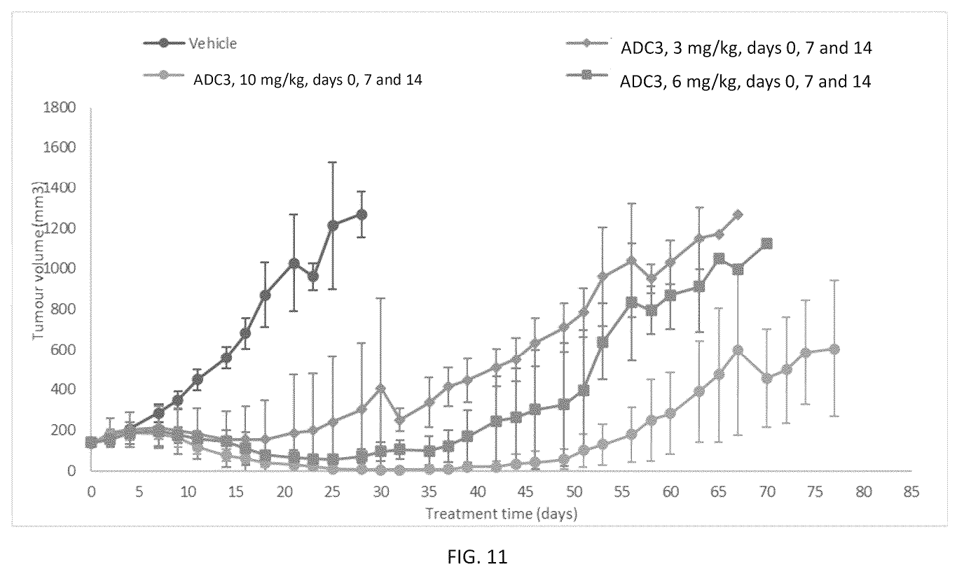

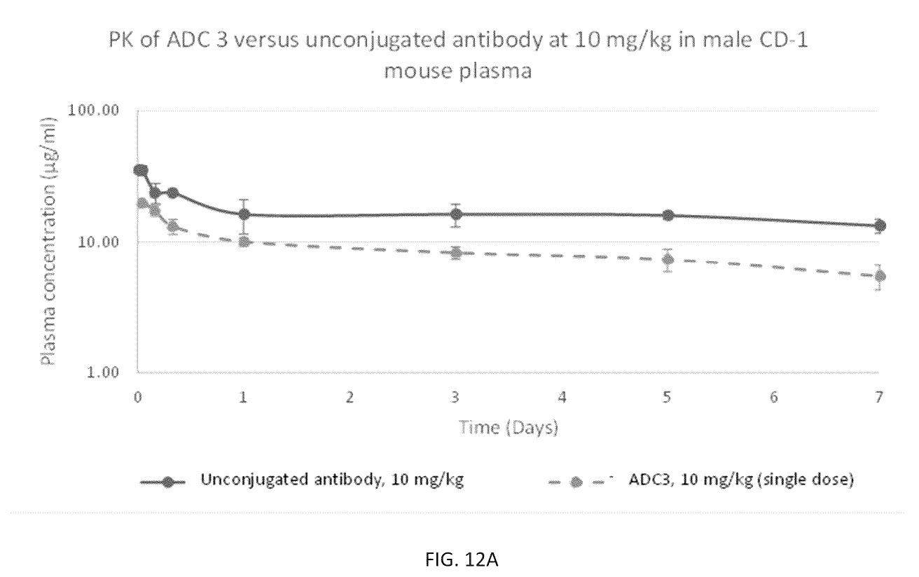

ADC3 was examined in a MDA-MB-231 (Triple Negative Breast Cancer), CDCP1+model after three doses at 10 mg/kg, 6 mg/kg, and 3 mg/kg and was found to reduce the rate of tumor growth compared to vehicle (FIG. 11).

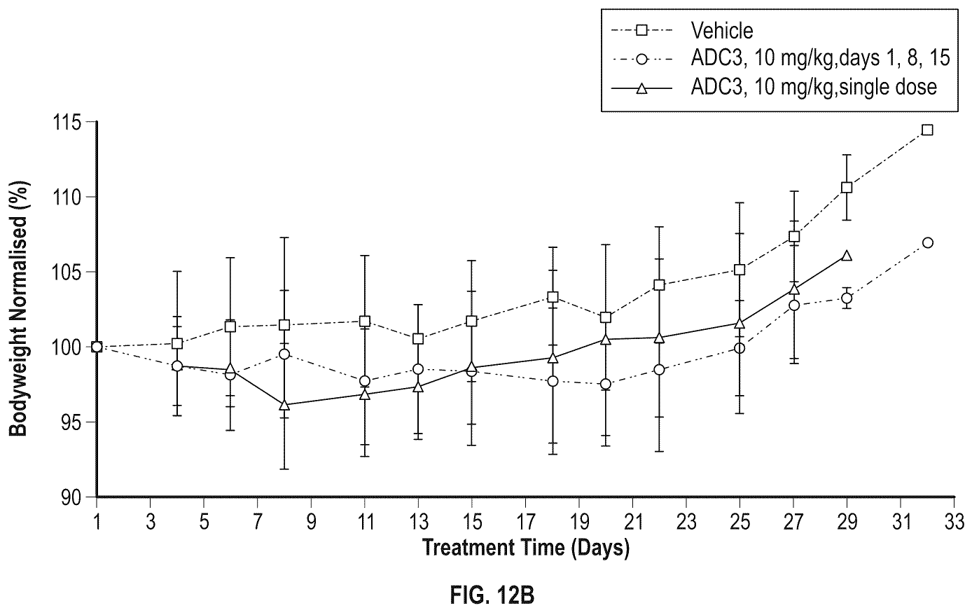

The PK profile of unconjugated mAb and ADC3 was examined in male CD1 mouse plasma. PK profile of the ADC was favourable, with little difference in clearance between mAb and ADC observed (FIG. 12).

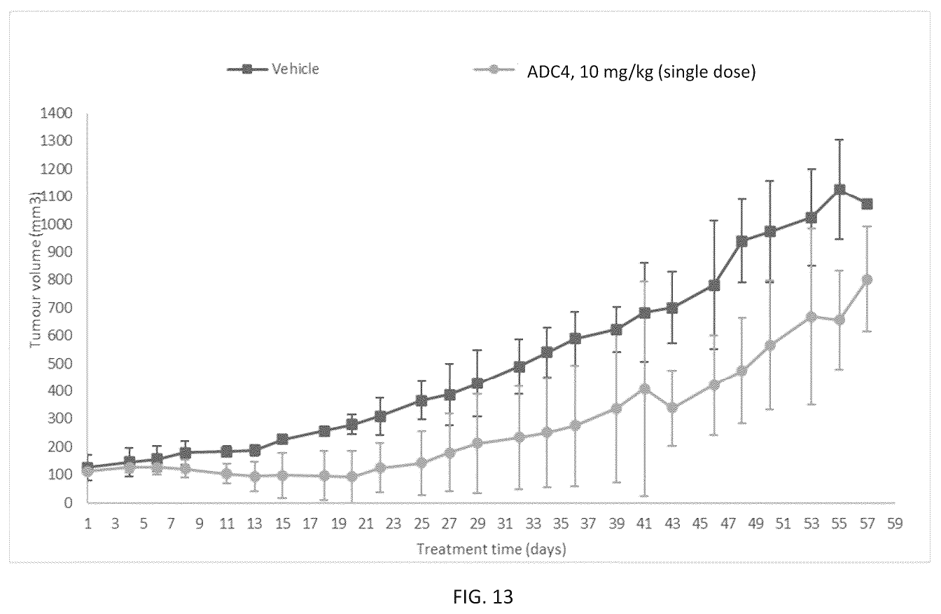

ADC4 was examined in a MDA-MB-231 (Triple Negative Breast Cancer), CDCP1+model at 10 mg/kg single dose and was found to reduce the rate of tumor growth compared to vehicle (FIG. 13).

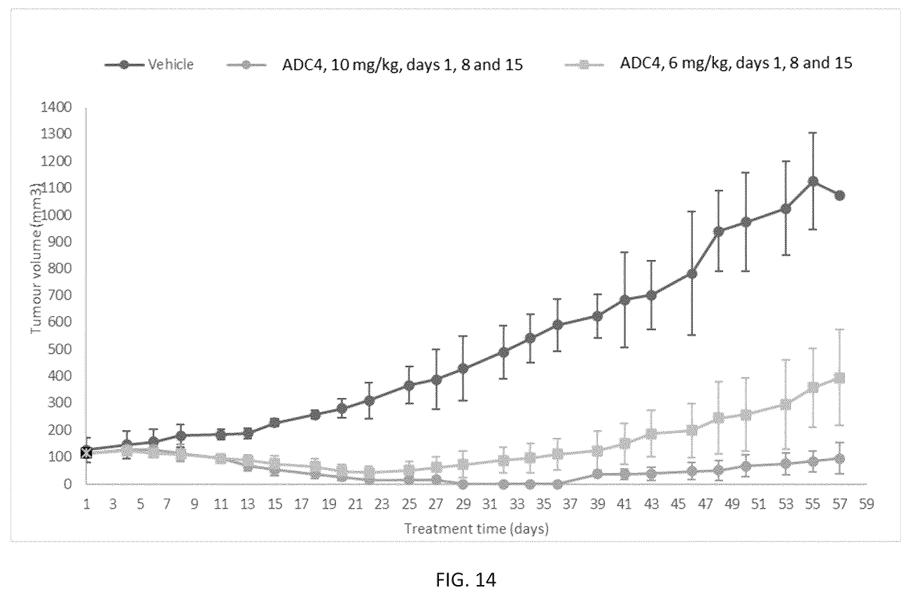

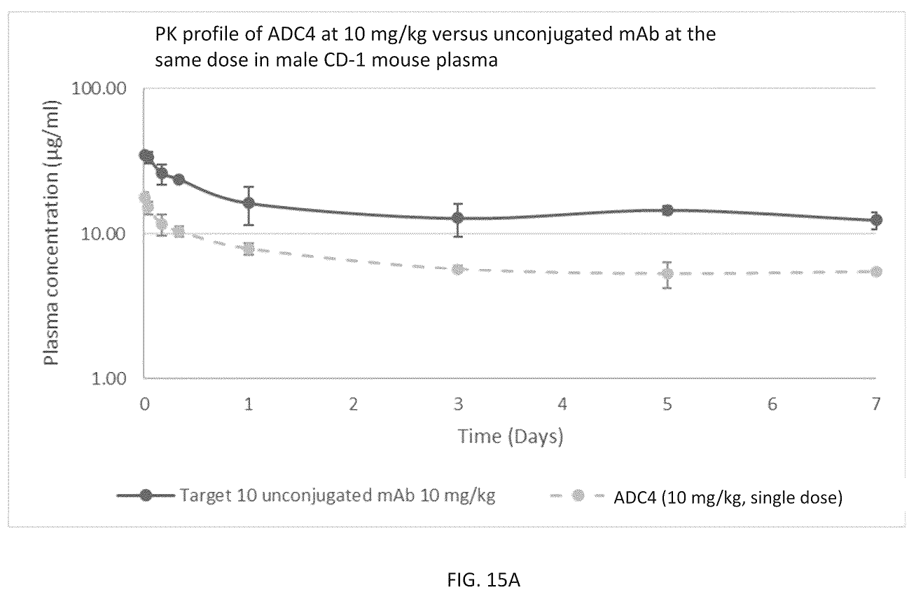

ADC4 was examined in a MDA-MB-231 (Triple Negative Breast Cancer), CDCP1+model after three doses at 10 mg/kg and 6 mg/kg and was found to reduce the rate of tumor growth compared to vehicle (FIG. 14).

The PK profile of unconjugated mAb and ADC4 was examined in male CD1 mouse plasma. PK profile of the ADC4 was favourable, with little difference in clearance between mAb and ADC4 observed (FIG. 12).

### Conjugation of 27 to IgG1 Antibody

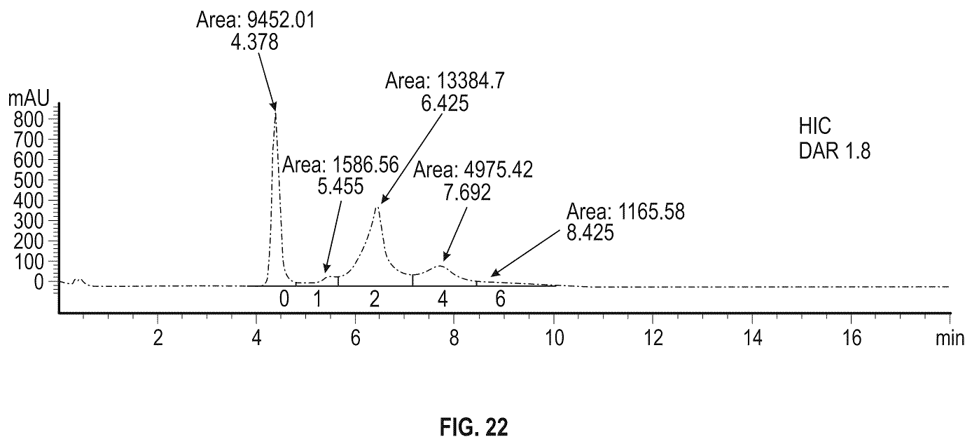

27 was conjugated to an IgG1 antibody targeted to CDCP1 in a stochastic manner to prepare ADC1. DAR (Drug Antibody Ratio) assignment was possible through HIC (FIG. 22). Average DAR was calculated as 1.8

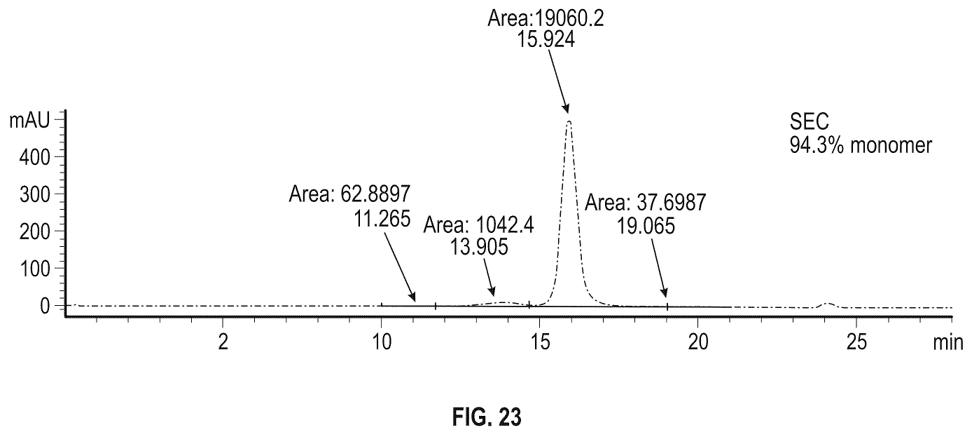

The conjugation process caused no significant aggregation compared to the starting antibody with ADC of 94.3% monomer produced (FIG. 23).

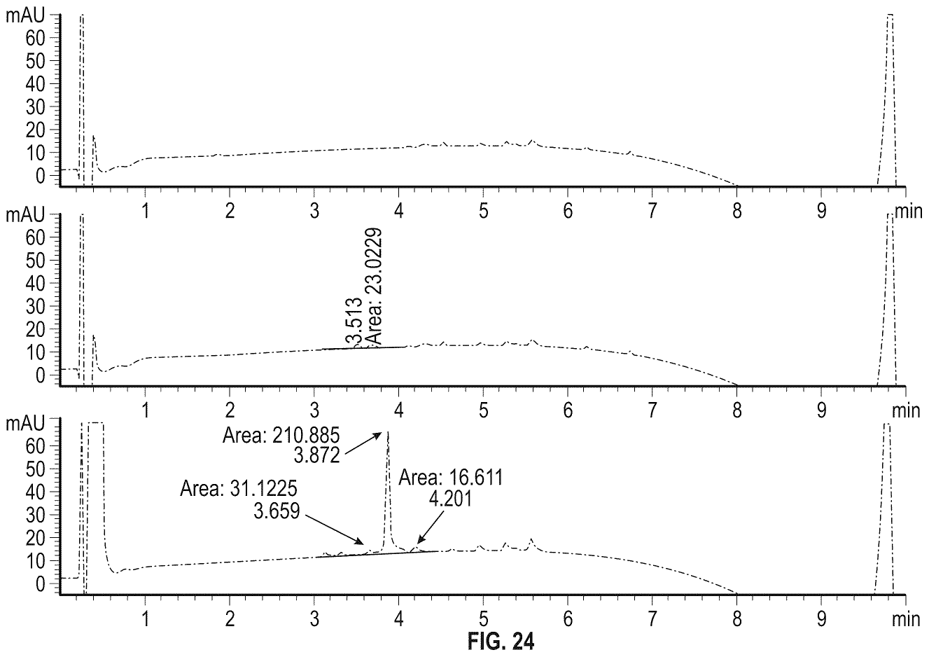

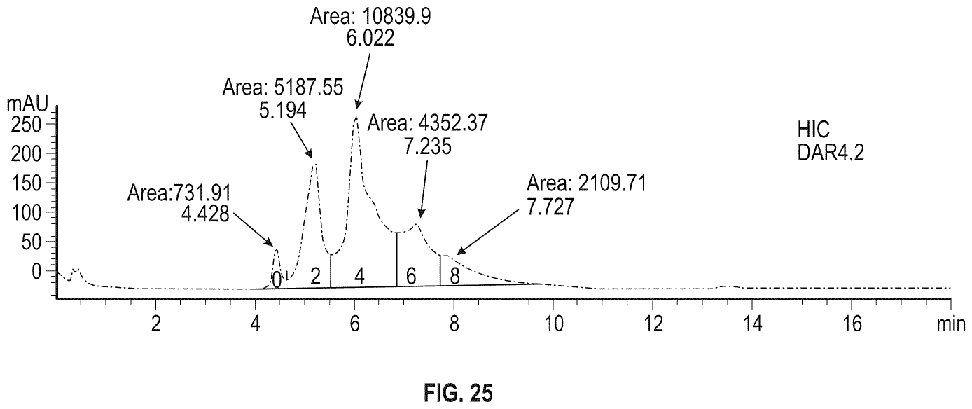

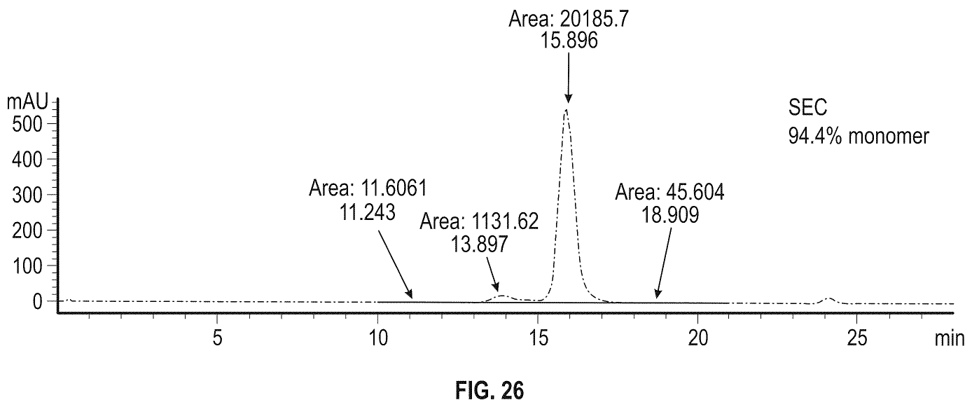

No free toxin linker could be detected in the ADC sample shown in (FIG. 24).

### Conjugation of 20 to CDCP1 mAb

20 was conjugated to an IgG1 antibody targeted to CDCP1 in a stochastic manner to prepare ADC2 forming an ADC with average DAR of 4.2. The conjugation process caused no significant aggregation compared to the starting antibody.

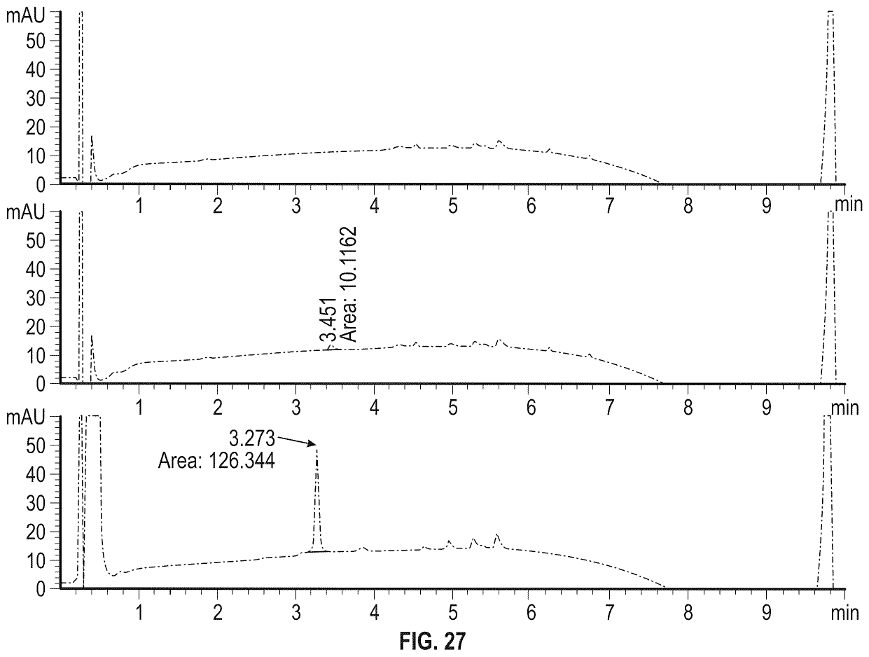

Limited free toxin linker could be detected in the ADC sample, as shown in FIG. 27.

### Conjugation of 37 to CDCP1 mAb (Forming ADC7)

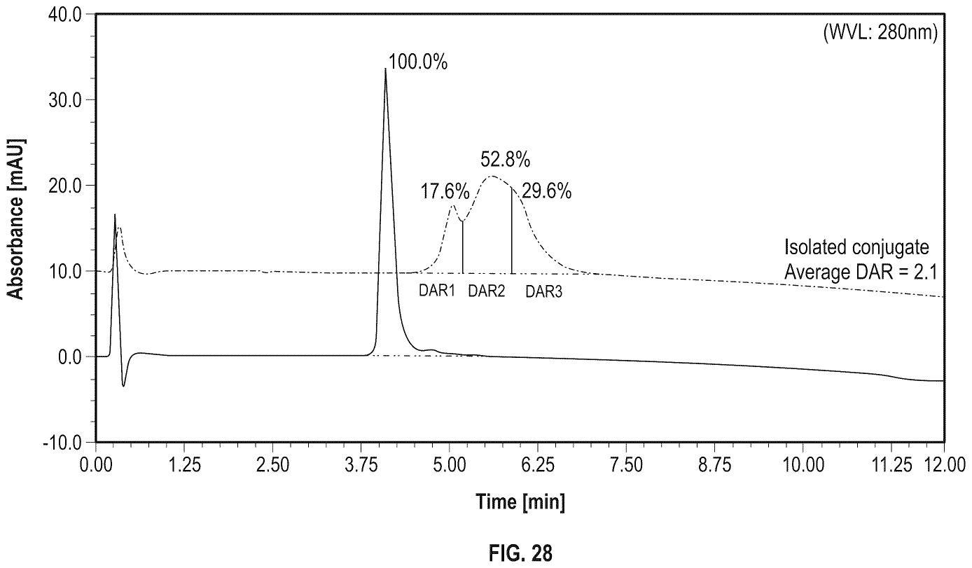

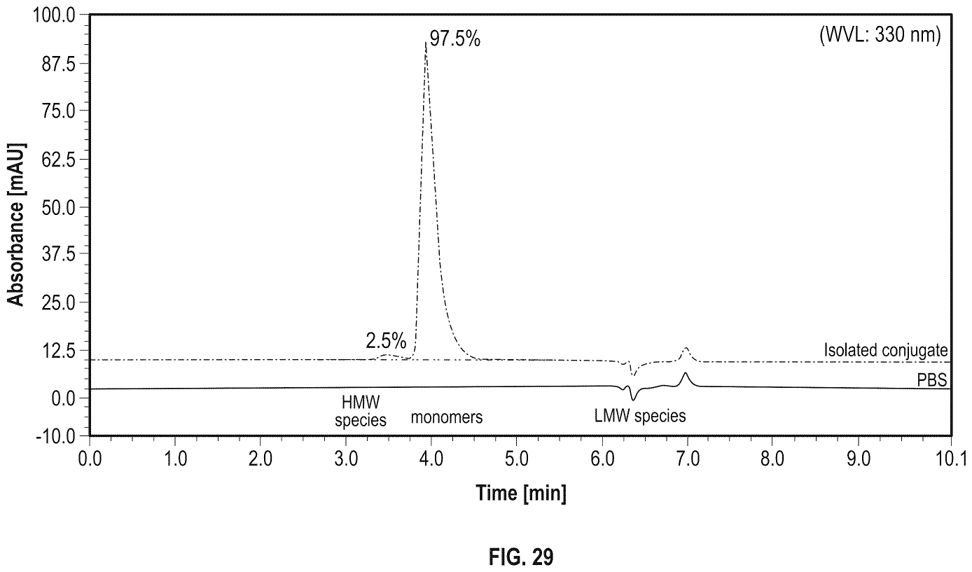

33 was conjugated to an IgG1 antibody targeted to CDCP1 in a stochastic manner to prepare ADC7 forming an ADC with average DAR of 2.1 (FIG. 28). The ADC contained 97.5% monomer (FIG. 29).

### In Vivo Tolerability of ADCs

The CDCP1 mAb is not cross-reactive with the murine antigen, so formal maximum tolerated doses were not determined. The following doses shown in Table 3 were tolerated and weight loss was <10%:

TABLE 3
ADC Doses

| | Highest dose tested in mice |
|---|---|
| ADC | (single dose, mg/kg) |
| ADC1 | 15 |
| ADC2 | 50 |
| ADC3 | 80 |
| ADC4 | 80 |

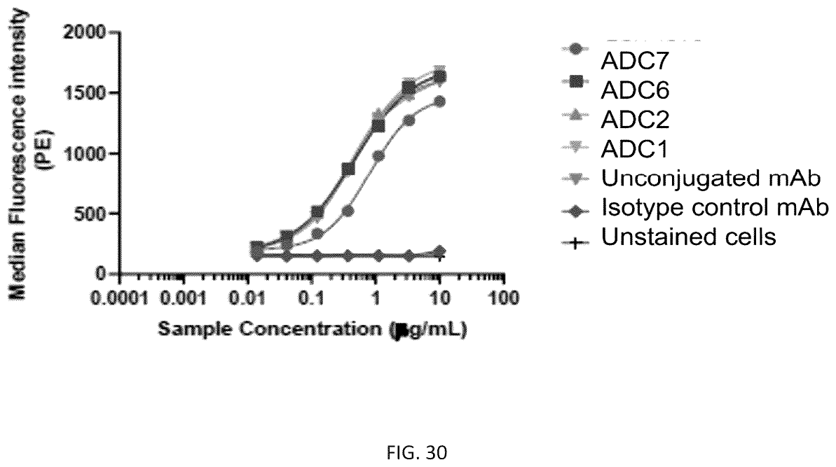

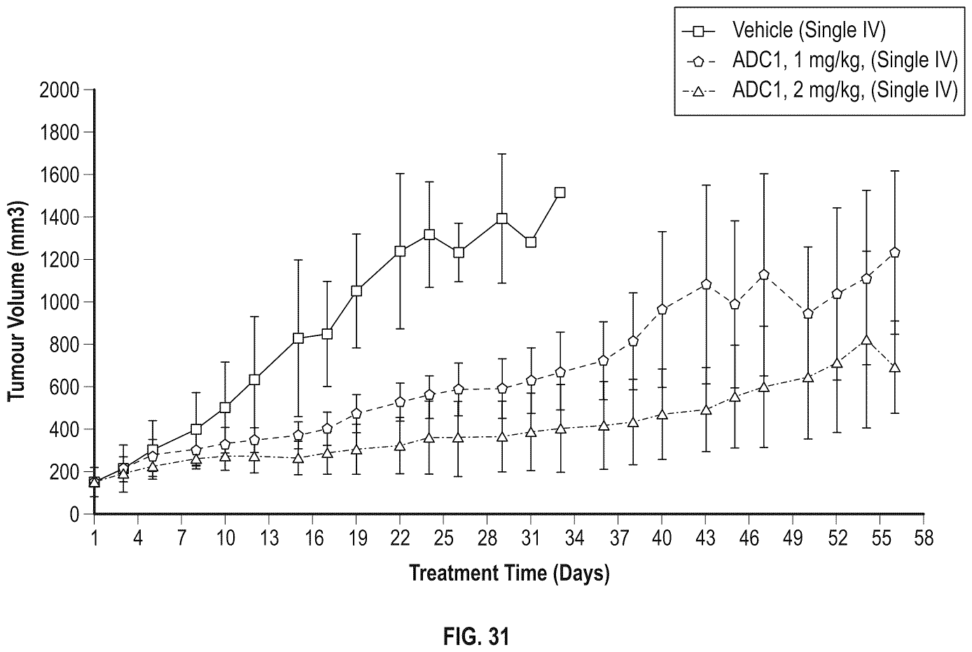

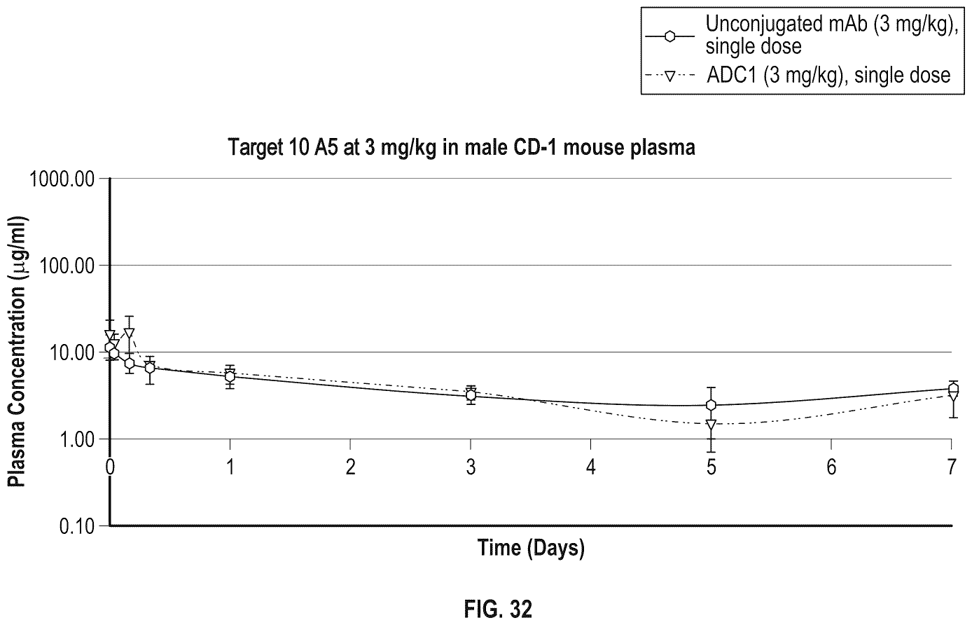

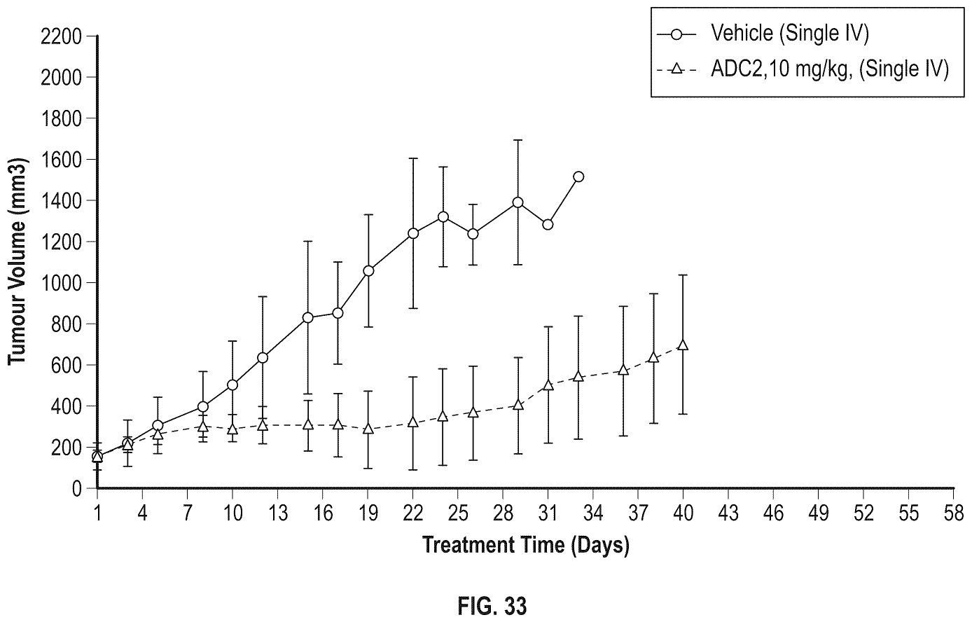

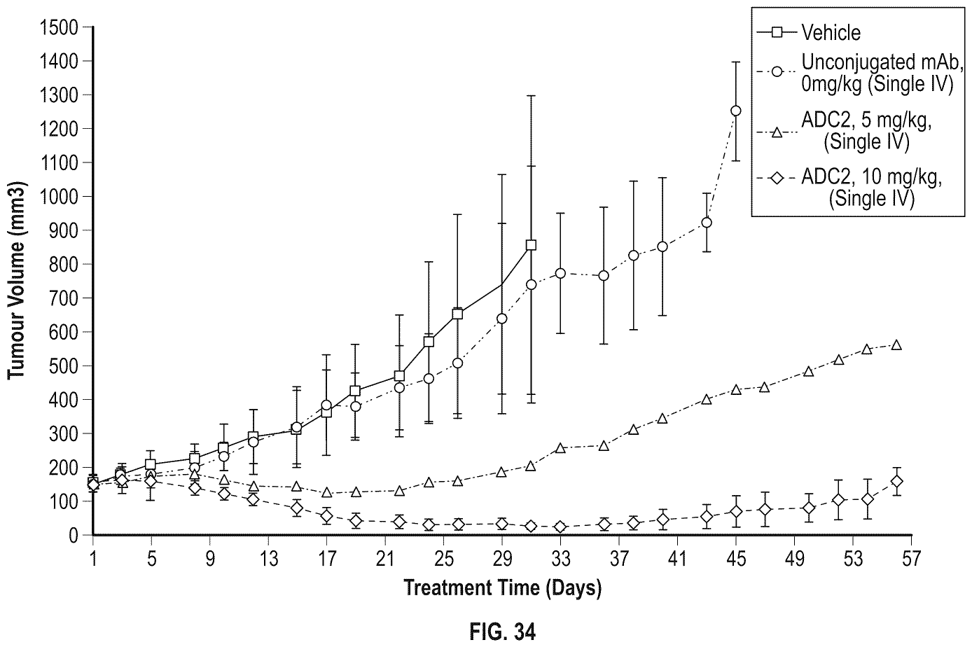

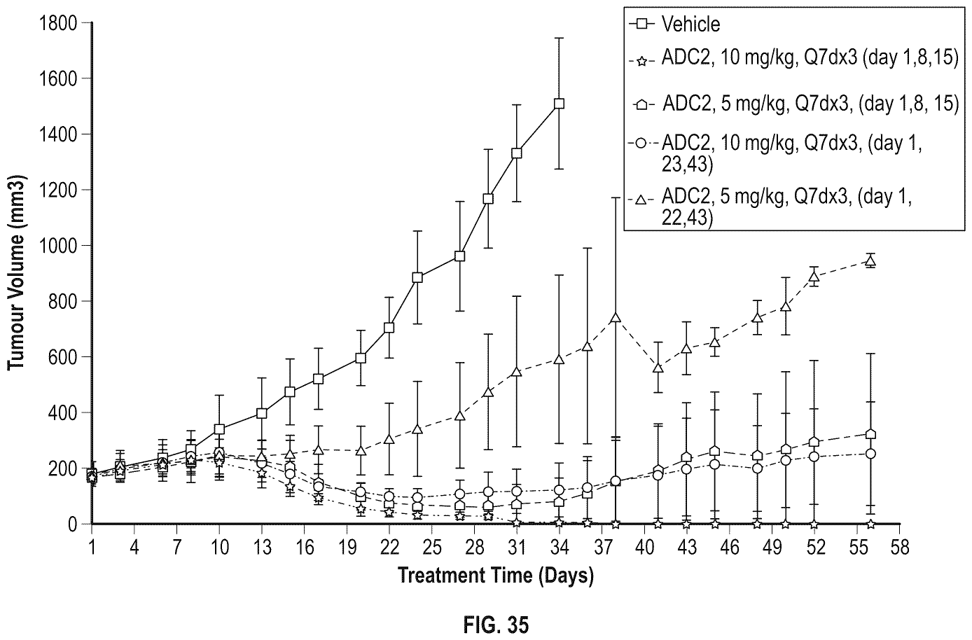

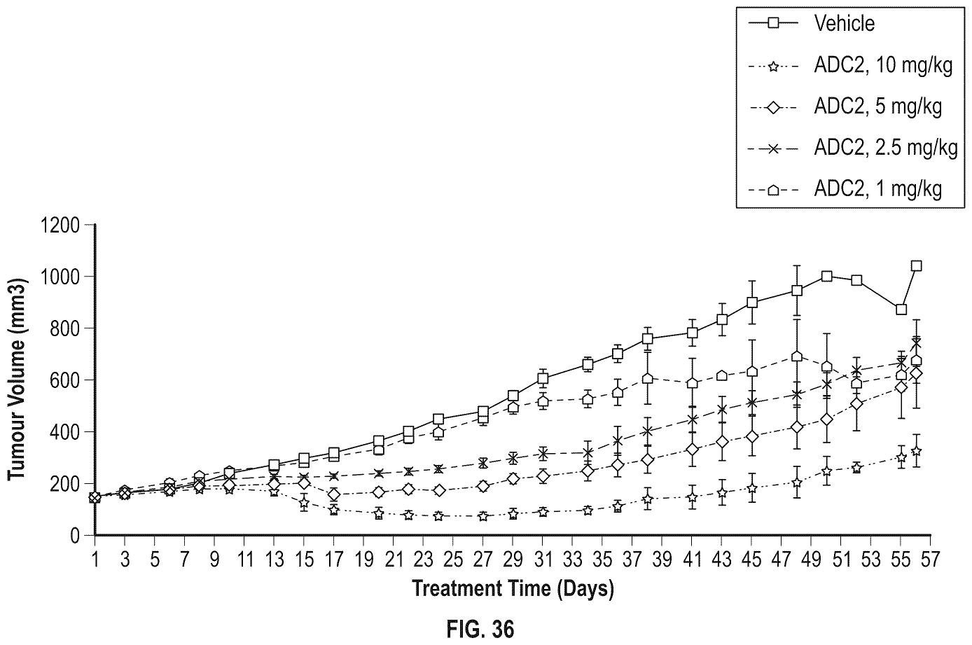

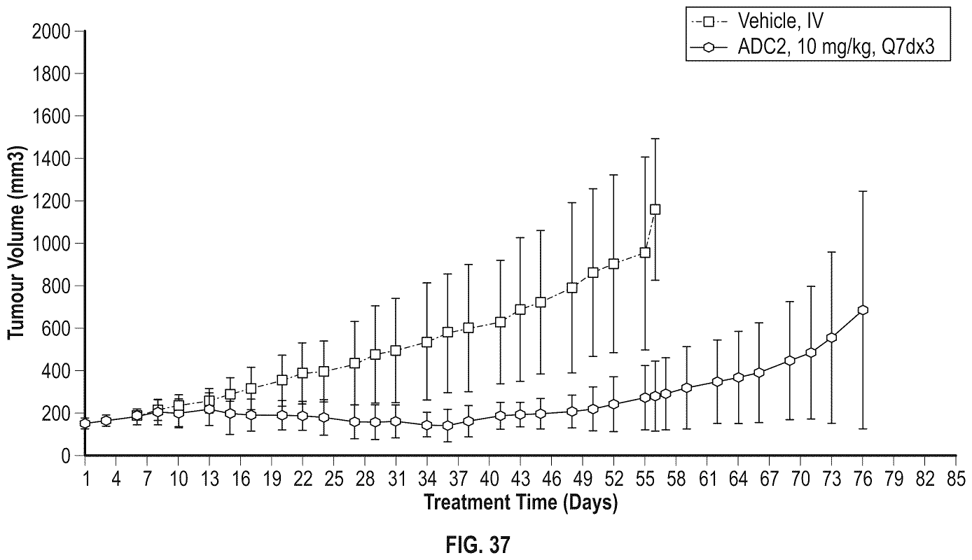

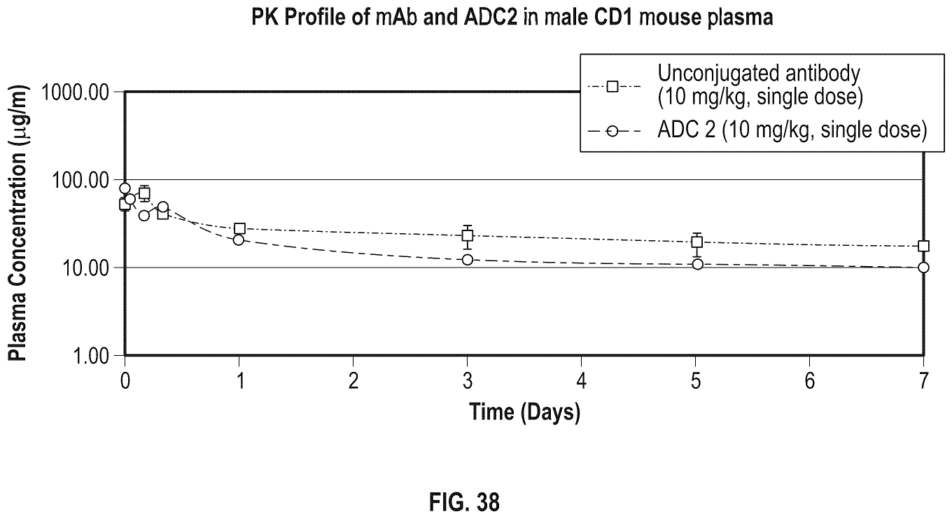

FIG. 30 ilustrates the binding affinity of prepared ADCs to antigen positive cells. FACS data illustrating binding of ADCs to antigen positive cell-line (A427). All ADCs has similar binding affinity compared to unconjugated CDCP1 mAb. Data also illustrate that unconjugated, non-targeted isotype control mAb did not bind to the antigen. FIG. 31 illustrates mean tumour volume versus time after one dose of ADC1 (Day 1) against K562. FIG. 32 illustrates the PK Profile of mAb and ADC1 in male CD1 mouse plasma. FIG. 33 illustrates mean tumour volume versus time after one dose of ADC2 (Day 1) against K562. FIG. 34 illustrates mean tumour volume versus time after one dose of ADC2 (Day 1) against MDA-MB-231 at both 5 and 10 mg/kg. Complete regression was observed at the higher dose with no weight loss. Unconjugated mAb had negligible effect, indicating a targeted cell-killing ability of the ADC. FIG. 35 illustrates mean tumour volume versus time after multiple doses of ADC2 (either Days 1, 8 and 15 or Days 1, 22 and 43) against MDA-MB-231 at both 5 and 10 mg/kg. Complete regression was observed at the higher dose with no weight loss. Unconjugated mAb had negligible effect, indicating a targeted cell-killing ability of the ADC. FIG. 36 illustrates mean tumour volume versus time after a single dose of ADC2 (Day 1) against PC3 at doses from 1 mg/kg to 10 mg/kg. Concentration-dependent regressions were observed with no weight loss. FIG. 37 illustrates mean tumour volume versus time after three doses of ADC2 (days 1, 7 and 14) against A427 at 10 mg/kg. FIG. 38 illustrates PK Profile of mAb and ADC2 in male CD1 mouse plasma. PK profile of the ADC is favourable, with little difference in clearance between mAb and ADC observed.

### Methodology

### 1.1 DNA Footprinting

The preparation of the TyrT DNA fragment has been previously described. Briefly, the sequence which had been cloned into the BamHI site of pUC18 was obtained by cutting with HindIII and EcoRI. Radiolabelled DNA fragments were prepared by filling in the 3′-end of the HindIII site with [α-³²P]dATP using Klenow DNA polymerase (exo-).

The radiolabelled DNA fragment was separated from the remainder of the plasmid DNA on a 6% non-denaturing polyacrylamide gel. The gel (20 cm long, 0.3 mm thick) was run at 400 V in 1×TBE running buffer for about 1-2 h, until the bromophenol blue had run most of the way down the gel. The glass plates were separated and the position of the labelled DNA fragment was established by short (1 min) exposure to an X-ray film. The relevant band was then cut from the gel and the radiolabelled DNA eluted by adding 300 μL 10 mM Tris-HCl, pH 7.5 containing 0.1 mM EDTA and gently agitating overnight at room temperature. The eluted DNA was finally precipitated with ethanol and re-suspended in a suitable volume of 10 mM Tris-HCl, pH 7.5 containing 0.1 mM EDTA buffer so as to give at least 10 counts per second/μL on a hand-held Geiger counter. With fresh plasmid and α-³²P-dATP this process typically generated about 150 μL of radiolabelled fragment DNA. The absolute concentration of the DNA is not important, and it is typically lower than 10 nM.

Footprinting reactions were performed as using previously described methods using the DNA fragments HexA and HexB, which together contain all 64 symmetrical hexanucleotide sequences, and MS1 that contains all possible 134 tetranucleotide sequences. The DNA fragments were obtained by cutting the parent plasmids with HindIII and SacI (for HexA and MS1) or EcoRI and PstI (for HexB), and were labelled at the 3′-end of the HindIII or EcoRI sites with [α-³²P]dATP using reverse transcriptase or exo-Klenow fragment. After gel purification, the radiolabelled DNA was dissolved in 10 mM Tris-HCl pH 7.5 containing 0.1 mM EDTA, at a concentration of about 10 c.p.s per μL as determined on a hand held Geiger counter. 1.5 μL of radiolabelled DNA was mixed with 1.5 μL ligand that had been freshly diluted in 10 mM Tris-HCl pH 7.5, containing 10 mM NaCl. The complexes were left to equilibrate for at least 12 hours before digesting with 2 μL DNase I (final concentration about 0.01 units/mL). The reactions were stopped after 1 minute by adding 4 μL of formamide containing 10 mM EDTA and bromophenol blue (0.1% w/v). The samples were then heated at 100° C. for 3 minutes before loading onto 8% denaturing polyacrylamide gels containing 8 M urea. Gels were fixed in 10% acetic acid, transferred to 3 MM paper, dried and exposed to a phosphor screen overnight, before analysing with a Typhoon phosphorimager

### 2. Cross-Linking Assay

### 2.1. Preparation of Ligand-DNA Complexes

Radiolabelled DNA (1.5 L) was mixed with 1.5 μL ligand solution of various concentrations (10 μM-10 nM) and incubated overnight at 37° C.

### 2.2 Cross-Linking Assay

After overnight incubation, the samples were mixed with 7 μL loading solution (80% formamide containing 10 mM EDTA, 10 mM NaOH, 0.1% bromophenol blue) and incubated at 65° C. for 5 min. Control 1 (C1) for native double-stranded DNA consisted of 1.5 μL labelled DNA, 1.5 μL 10 mM Tris-HCl, pH 7.5 containing 0.1 mM EDTA and 7 μL 1× loading dye. Control 2 (C2) for denatured native single-stranded DNA was composed of 1.5 μL labelled DNA, 1.5 μL 10 mM Tris-HCl, pH 7.5 containing 0.1 mM EDTA which was incubated at 65° C. for 5 min. Control 3 (C3) for native double-stranded DNA consisted of 1.5 μL labelled DNA, 1.5 μL 10 mM Tris-HCl, pH 7.5 containing 0.1 mM EDTA and 7 μL SSB. Control 4 (C4) for denatured native single-stranded DNA was composed of 1.5 μL labelled DNA, 1.5 μL 10 mM Tris-HCl, pH 7.5 containing 0.1 mM EDTA and 7 μL SSB which was incubated at 65° C. for 5 min. Separation was performed on a 7.5% denaturing polyacrylamide gel (20 cm long, 0.3 mm thick) at 500V for about 4 h until the dye reached the bottom of the gel. The gel plates were then separated, the gels fixed by immersing in 10% (v/v) acetic acid, followed by transfer to Whatmann 3 MM paper and drying under vacuum at 80° C. The dried gel was then exposed to a phosphorimager screen overnight before scanning using a Typhon FLA 7000 instrument.

### Example 3: In Vivo ADC Studies

A major focus of this study is to develop a treatment for cancer, particularly cancer types that are known to express the CDCP1 antigen, one of which is triple-negative breast cancer (TNBC). With this goal in mind, initial animal studies of the CDCP1-targeted exatecan conjugate (ACD3) began with assessment of efficacy in a MDA-MB-231 xenograft model. Subjects were administered with either a single-dose at either 6 or 10 mg/kg (FIG. 10) or three weekly doses at 3, 6, or 10 mg/kg (FIG. 11), both at a DAR of 8. Over the four-week experiment, tumor volumes were measured every other day.

Treatment with ADC3 resulted in a dose-dependent tumor volume reduction relative to vehicle control with complete regression under certain conditions. For the single-dose regimen, pronounced effects were initially observed at both tested concentrations, including arrest of tumor growth for approximately 14 days. However, growth begins again, after this period of arrest, though some dose-dependency was observed in the rate of post-arrest growth (FIG. 10).

These results led to the assessment of a weekly multi-dose regimen in the same model system. As before, tumor growth is arrested after the initial dose at all tested concentrations relative to vehicle control (FIG. 11). With subsequent administrations at days 7 and 14, both the 6 and 10 mg/kg concentrations trended toward regression, while the 3 mg/kg multi-dose continued to exhibits significant reduction relative to control. Complete regression was observed to day 50 at the higher dose (10 mg/kg), with regrowth occurring thereafter.

The specific pharmacokinetics of ADC3 and impact of either dosing regimen on subject body weight were next assessed. First, the half-life of 10 mg/kg ADC3 was compared to the same concentration of unconjugated antibody, with male CD-1 mice being administered a single dose of each and plasma concentration being measured daily. The results show that conjugation only slightly reduces half-life compared to the unconjugated antibody—the half-life of ADC3 is 182 hours as compared to 224 hours for the control (FIG. 12A).

Next, animal body weight was measured using either the single-dose or weekly multi-dose regimen (administrations of 10 mg/kg ADC3 at days 1, 8 and 15; DAR of 8) in healthy animals. No drastic differences were observed under either condition as compared to vehicle control over a 33-day observation window (FIG. 12B). Indeed, body weights steadily increased under all conditions, though animals administered the single-dose or multi-dose regimens experienced an initial drop in weight over approximately the first 8 days of treatment before beginning to gain.

Having seen positive results at a DAR of ~8, additional studies were undertaken using a lower DAR. MDA-MB-231 xenograft mice were administered either vehicle or 10 mg/kg ADC4 as part of the single-dose regimen. Tumor growth was measured every other day. Similarly to the higher DAR, a single dose of ADC4 resulted in arrest of tumor growth for a period of approximately 20 days before growth recurred (FIG. 13).

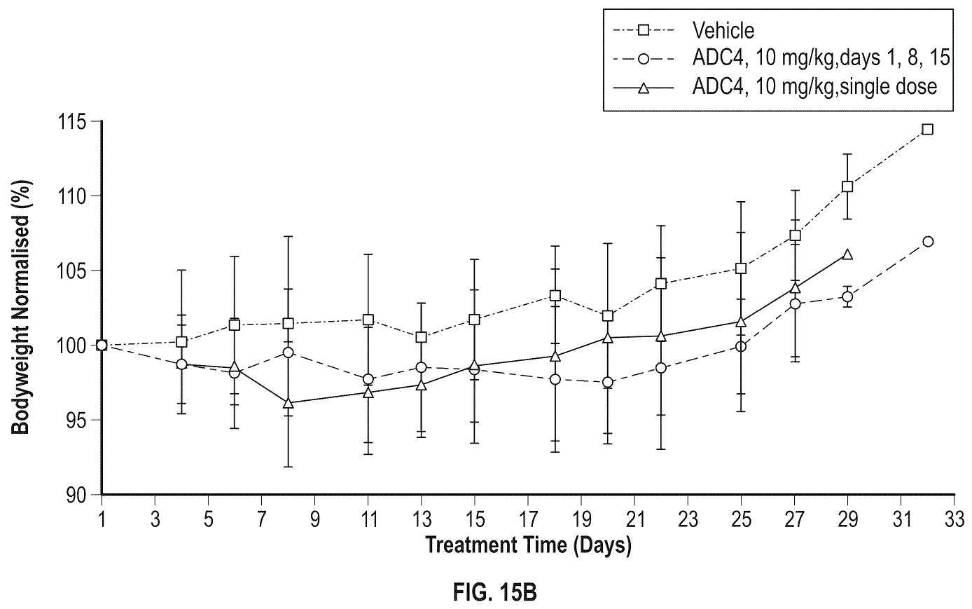

A multi-dose regimen was also administered to mice (three weekly administrations at 6 or 10 mg/kg of ADC4). As in the higher DAR experiments, a dose-dependent effect was observed (FIG. 14). Further, the higher concentration led to complete regression of tumor growth. Pharmacokinetics analysis found that ADC4 remained detectable in male CD-1 mouse plasma for at least seven days, though at levels below that of an unconjugated antibody control (FIG. 15A). Similar to previous results, animal body weight was not drastically reduced with either a single-dose or multi-dose regimen at a DAR of 4 with animals steadily gaining weight over time (FIG. 15B).

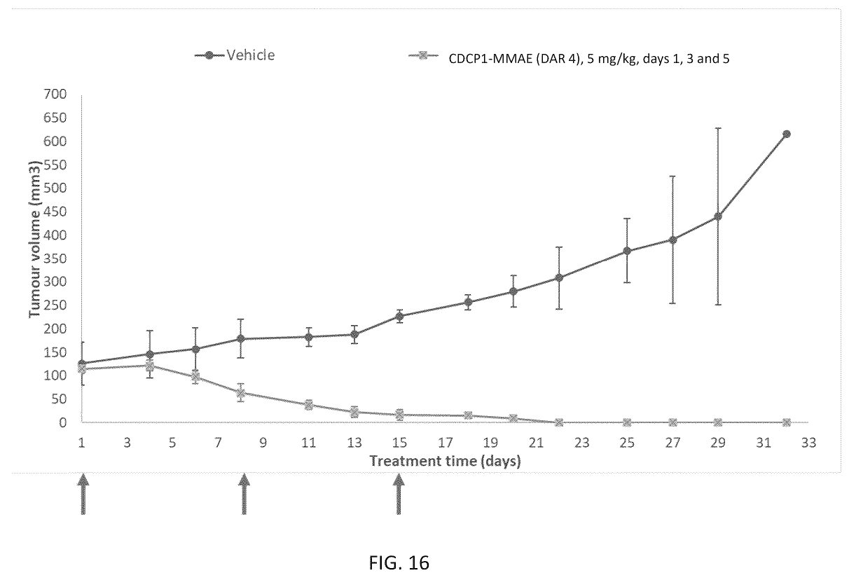

Finally, the previously observed anti-tumorigenic effects were the basis for testing a related CDCP1-targeted conjugate in the same TNBC model. CDCP1-vcMMAE at 5 mg/kg (DAR of 4) was administered in a modified multi-dose regimen, in which administration occurred at days 1, 3, and 5. Results showed a reduced tumor volume as compared to vehicle control and complete regression by the end of the experimental window of 32 days (FIG. 16). As with other experiments, no impact on body weight was observed, and antigen copy number of Target 10 was approximately 89,438.

### Example 4: Biological and Biophysical Characterisation of Free Payloads

### Biophysical Characterisation

### 1. DNA Cross-Linking Assay

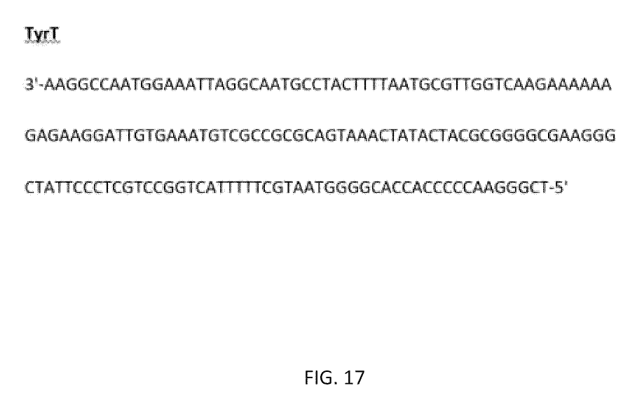

The ability of 26 to cross-link DNA was evaluated using an assay involving a linear double-stranded TyrT fragment (FIG. 17). The PBD dimer Talirine (SGD1882) was used as a positive control, as PBD dimers have previously been shown to cross-link DNA.

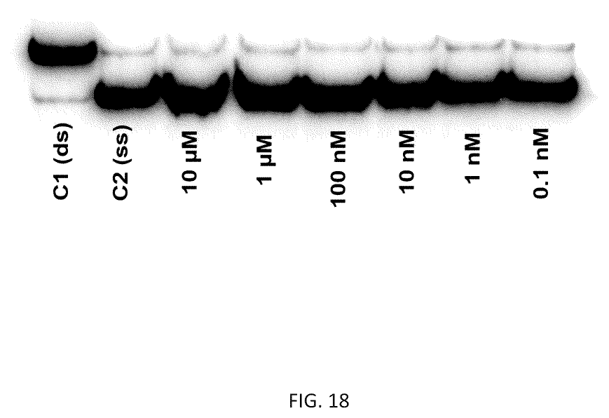

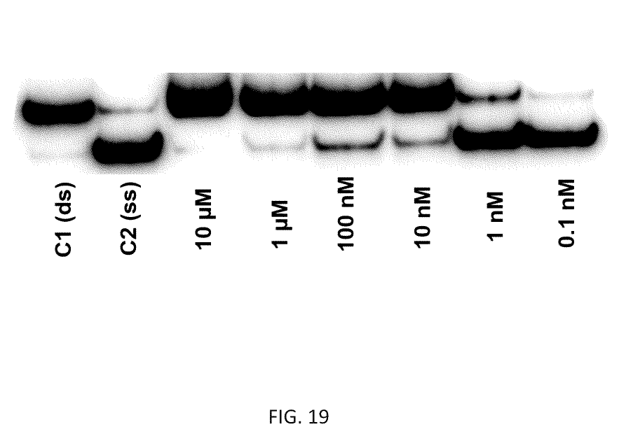

Following denaturation conditions (treatment with formamide and heating at 65° C. for 5 min), the DNA strands were completely separated (see control C2, FIGS. 18 and 19). The presence of an interstrand cross-link holds the denatured strands in close proximity, and cross-linked adducts therefore run as double-stranded DNA on polyacrylamide gel.

Both compounds were tested at six different concentrations, and the assay was repeated twice. The cross-linking ability of 26 is shown in FIG. 18. Cross-links are not detectable at any concentration (i.e., from 10 μM to as low as 0.1 nM), whereas the PBD dimer Talirine produces cross-links at concentrations as low as 10 nM (FIG. 19). These results demonstrate that 26, and other PDD-based agents such as 19, are incapable of cross-linking DNA as they only contain one imine group, consistent with their proposed mono-alkylation mechanism of action.

### 2. DNA Footprinting

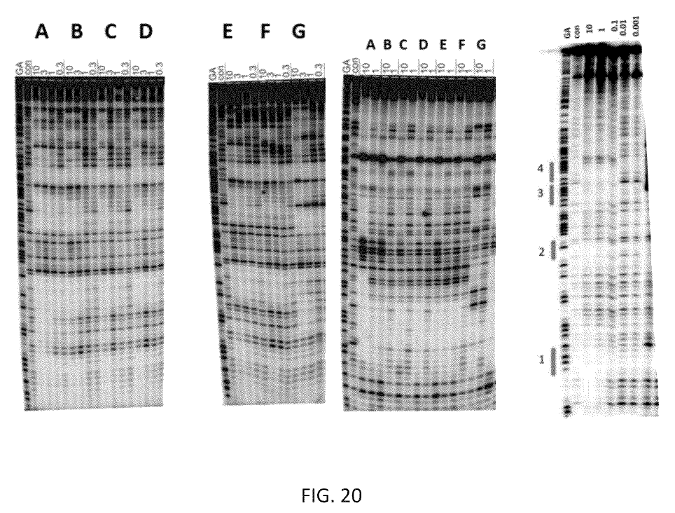

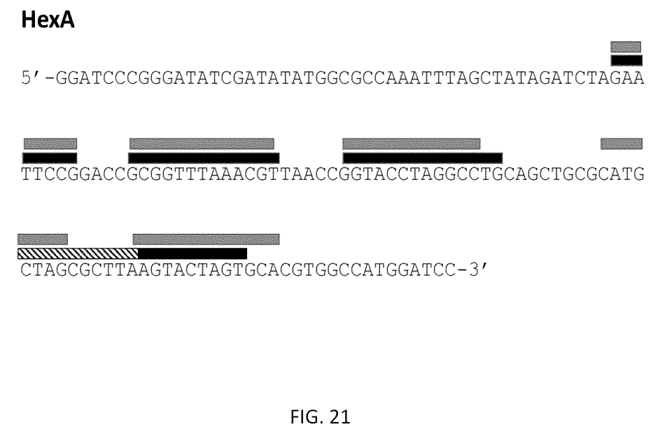

The DNA sequence selectivity profile of the molecules was investigated using a modification of a previously established DNA footprinting assay. Following an overnight incubation of the ligand-DNA complexes, the mixture was mixed with strand separation buffer containing 10 mM EDTA, 10 mM NaOH, 0.1% bromophenol blue, 80% formamide and incubated at 100° C. for 3 min. The mixture was then immediately cooled on ice and run on an 8% denaturing gel. Examination of the obtained gel (FIG. 20) shows footprints produced by the molecules on HexA DNA sequences. Interestingly, although the HexA DNA fragments contain multiple potential binding sites for 26 (i.e., multiple examples of potential G-alkylating sites), only five preferred sites in the case of HexA were observed during this experiment. These data also suggest that G-alkylators all act in a highly sequence selective manner with a different sequence selectivity profile to the PBD dimer Talirine (green blocks, FIG. 21). The possible adducts formed within the HexA sequences are shown in FIGS. 20 and 21, respectively.

### Summary of Biophysical

The biophysical data presented above provide strong evidence that 26 effectively stabilises DNA with a high degree of sequence-specificity. Although not wishing to be bound by any particular theory, these data suggest that the population of DNA adduct types derived may account for the cytotoxicity of this family of compounds in cells. Furthermore, DNA Footprinting studies indicate a degree of sequence selectivity for the class (both 17 and 26), with the DNA-binding site generally corresponding to XGXWWWW where X represents any base and W indicates adenine or thymine. Although not wishing to be bound by any particular theory, overall these data suggest that the potent cytotoxicity observed for the PDD class of payloads is directly related to their DNA-binding affinity and sequence selectivity.

### Example 5: In Vivo Efficacy and Toxicology Studies

The in vivo efficacy of ADC3 (DAR of 8) and CDCP1-vcMMAE (DAR of 4) against lung cancer and triple negative breast cancer (TNBC) was assessed in tumour xenograft mouse models.

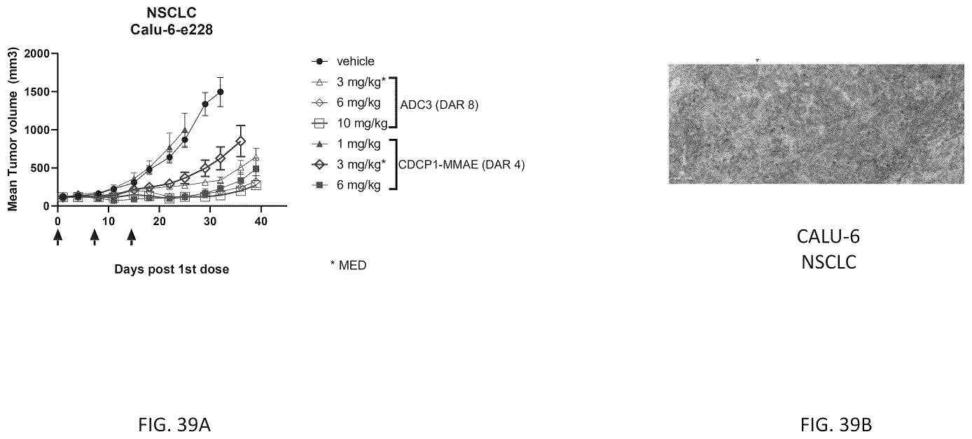

ADC3 (DAR of 8) was examined in a NSCLC CALU-6 (lung cancer) model after dosing at Q7dx3 at 3, 6, and 10 mg/kg and was found to reduce the rate of tumor growth compared to vehicle at each dosage amount (FIG. 39A). CDCP1-vcMMAE (DAR of 4) was examined in a NSCLC CALU-6 (lung cancer) model after dosing at Q7dx3 at 1, 3, and 6 mg/kg and was found to reduce the rate of tumor growth compared to vehicle at dosage amounts 3 and 6 mg/kg (FIG. 39A). An immunohistochemistry (IHC) image showing expression of CDCP1 in the NSCLC cell-line is shown in FIG. 39B.

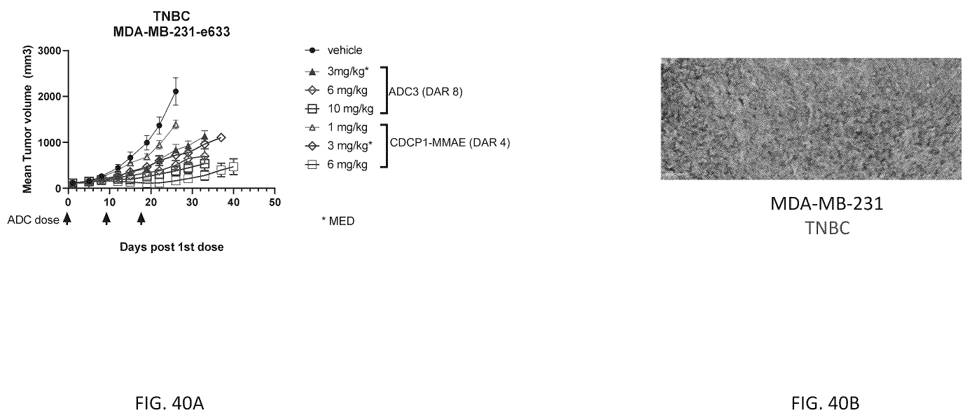

ADC3 (DAR of 8) was examined in a MDA-MB-231 (TNBC) model after dosing at Q7dx3 at 3, 6, and 10 mg/kg and was found to reduce the rate of tumor growth compared to vehicle at each dosage amount (FIG. 40A). CDCP1-vcMMAE (DAR of 4) was examined in a MDA-MB-231 (TNBC) model after dosing at Q7dx3 at 1, 3, and 6 mg/kg and was found to reduce the rate of tumor growth compared to vehicle at each dosage amount (FIG. 40A). An IHC image showing expression of CDCP1 in the TNBC cell-line pre-treatment is shown in FIG. 40B.

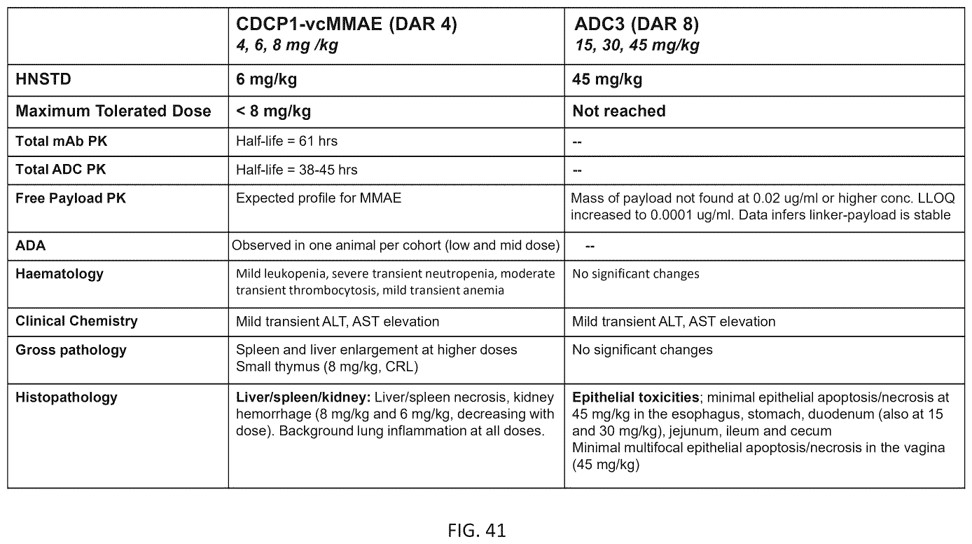

Data from the DRF and PK studies conducted in cynomolgus monkeys in FIG. 41 did not identify any serious issues using CDCP1-vcMMAE (DAR of 4) or ADC3 (DAR of 8), and while not wishing to be bound by any particular theory, any observed toxicities were deemed to be target independent and consistent with the payload's mechanism of action (MOA). No lung issues or interstitial lung disease (ILD) was observed for ADC3 (DAR of 8). TPS scoring (the number of positive tumour cells divided by the total number of viable tumor cells multiplied by 100%).

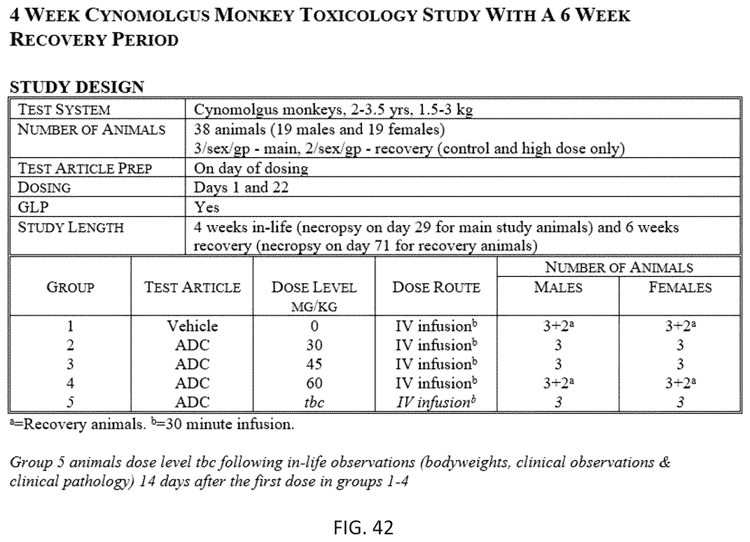

The proposed GLP toxicology study design framework for testing ADC3 (DAR of 8) in cynomolgus monkeys shown in FIG. 42 uses a staggered dosing arrangement and enables flexibility in in-life approach.

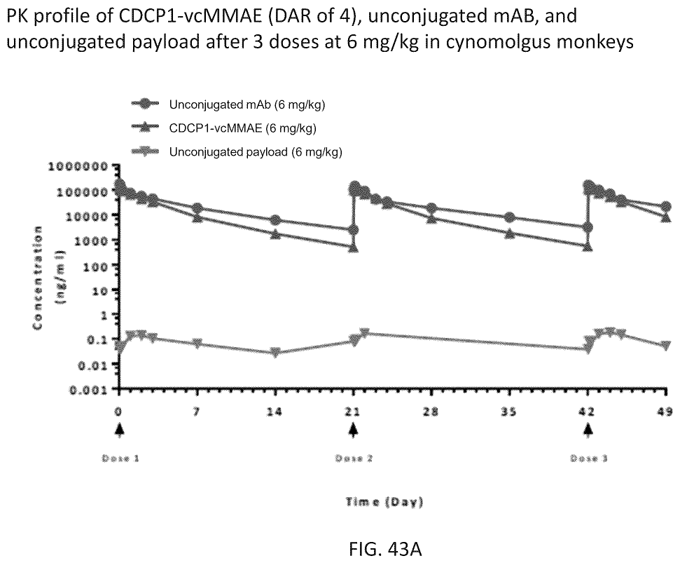

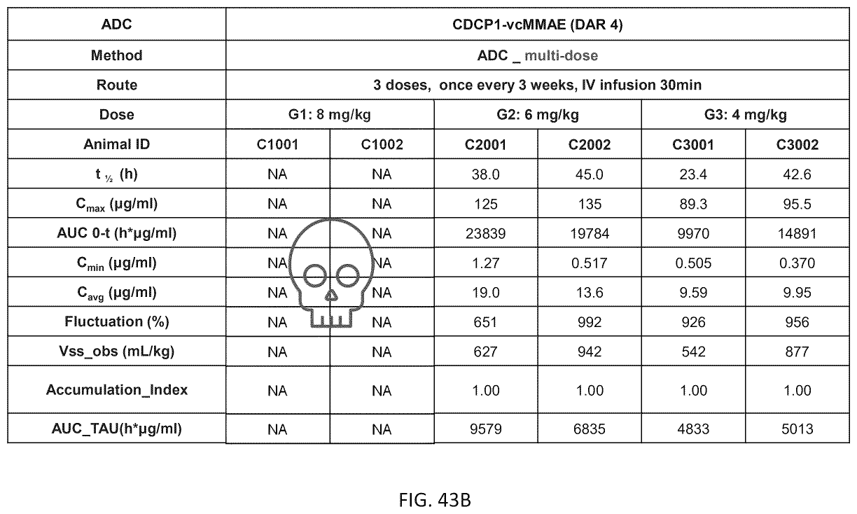

The PK profile of CDCP1-vcMMAE, unconjugated mAb, and unconjugated payload was examined in cynomolgus monkeys after 3 doses at 6 mg/kg (FIG. 43A). The half-life (t_{1/2}) of the total ADC CDCP1-vcMMAE was 38-45 hrs, and the t_{1/2} for the unconjugated mAb and unconjugated payload was 61 hrs and 87-110 hrs, respectively. The t_{1/2} values compare favorably to t_{1/2} values of other MMAE ADCs (Adcetris: ADC cyno t_{1/2}=45-72 hrs). Additional PK data after 3 doses of CDCP1-vcMMAE at 8 mg/kg, 6 mg/kg, and 4 mg/kg in cynomolgus monkeys is shown in FIG. 43B.

### Example 6: In Vitro Pharmacology and IHC mAb Assay Development

In vitro pharmacology was assessed in binding experiments of unconjugated CDCP1-mAb and ADCs to recombinant CDCP1 ECD.

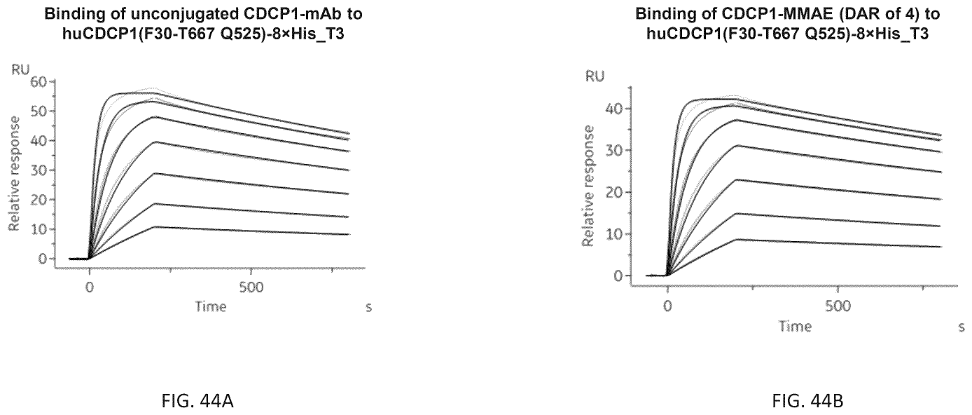

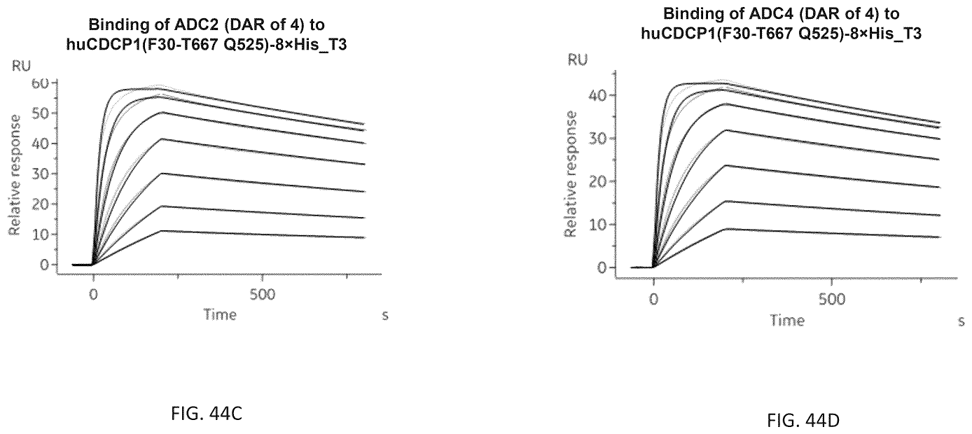

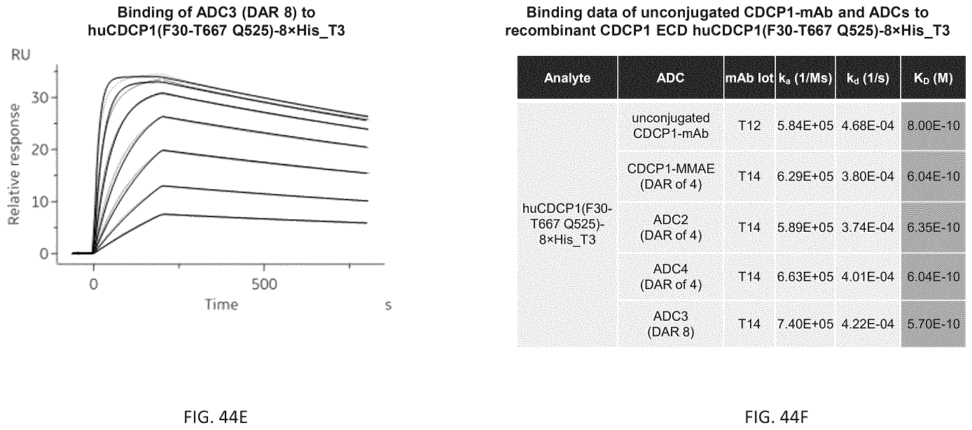

FIG. 44A is a graph of experimental data illustrating binding of unconjugated CDCP1-mAb to huCDCP1 (F30-T667 Q525)-8×His_T3. FIG. 44B is a graph of experimental data illustrating binding of CDCP1-vcMMAE (DAR of 4) to huCDCP1 (F30-T667 Q525)-8×His_T3. FIG. 44C is a graph of experimental data illustrating binding of ADC2 (DAR of 4) to huCDCP1 (F30-T667 Q525)-8×His_T3. FIG. 44D is a graph of experimental data illustrating binding of ADC4 (DAR of 4) to huCDCP1 (F30-T667 Q525)-8×His_T3. FIG. 44E is a graph of experimental data illustrating binding of ADC3 (DAR 8) to huCDCP1(F30-T667 Q525)-8×His_T3. FIG. 44F is a table summarizing additional binding data of unconjugated CDCP1-mAb and ADCs to recombinant CDCP1 ECD huCDCP1(F30-T667 Q525)-8×His_T3. The data shown in FIGS. 44A-44F demonstrates that the unconjugated CDCP1-mAb binds to CDCP1 ECD with a K_{D} of about 1 nM, as determined via SPR. The data also shows that conjugation of CDCP1-mAb to the payloads of MMAE, compound 20, and compound 30 did not alter K_{D}.

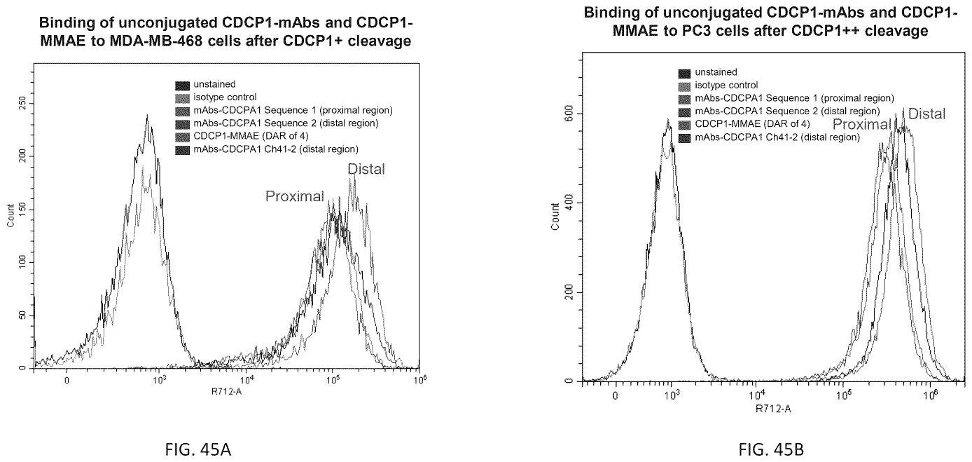

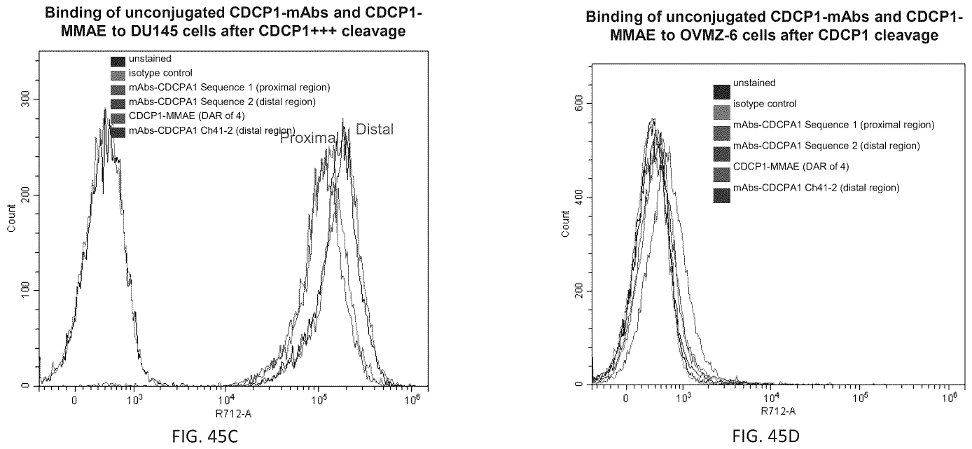

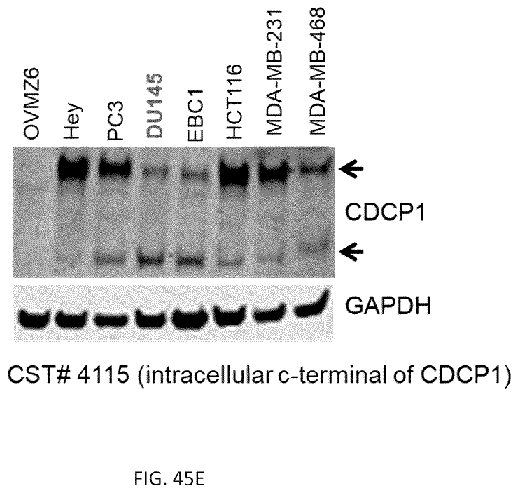

FIG. 45A is a graph of experimental data illustrating binding of unconjugated CDCP1-mAbs and CDCP1-vcMMAE to MDA-MB-468 cells after CDCP1+ cleavage. FIG. 45B is a graph of experimental data illustrating binding of unconjugated CDCP1-mAbs and CDCP1-vcMMAE to PC3 cells after CDCP1++ cleavage. FIG. 45C is a graph of experimental data illustrating binding of unconjugated CDCP1-mAbs and CDCP1-vcMMAE to DU145 cells after CDCP1+++ cleavage. FIG. 45D is a graph of experimental data illustrating binding of unconjugated CDCP1-mAbs and CDCP1-vcMMAE to OVMZ-6 cells after CDCP1 cleavage. FIG. 45E is an image of polyacrylamide gel binding assay. The data shown in FIGS. 45A-45E demonstrates that CDCP1-mAbs cleavage does not significantly impact in vitro binding regardless of whether mAbs bind to the membrane proximal or distal regions of CDCP1. mAbs-CDCPA1 Sequence 1 (proximal region), mAbs-CDCPA1 Sequence 2 (distal region), and mAbs-CDCPA1 Ch41-2 (distal region) antibodies did not show significant difference in binding to CDCP1+ cells. The sequences of the light chain variable region, heavy chain variable region, and CDRs (SEQ ID NOs: 15-22) of “Sequence 2” can be found in Table 2B.

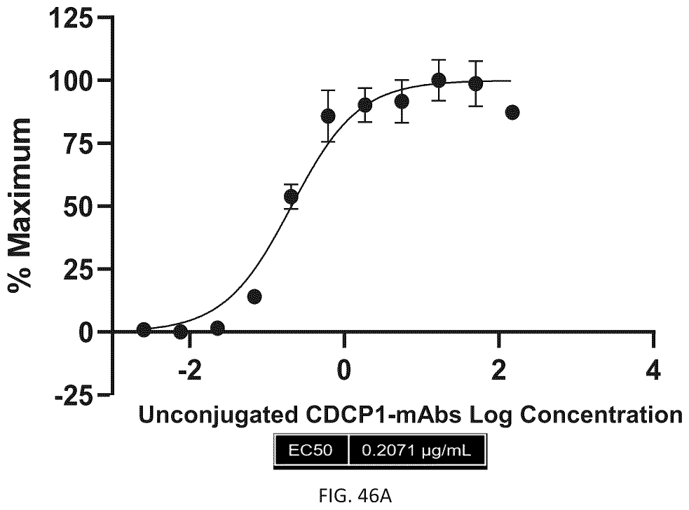

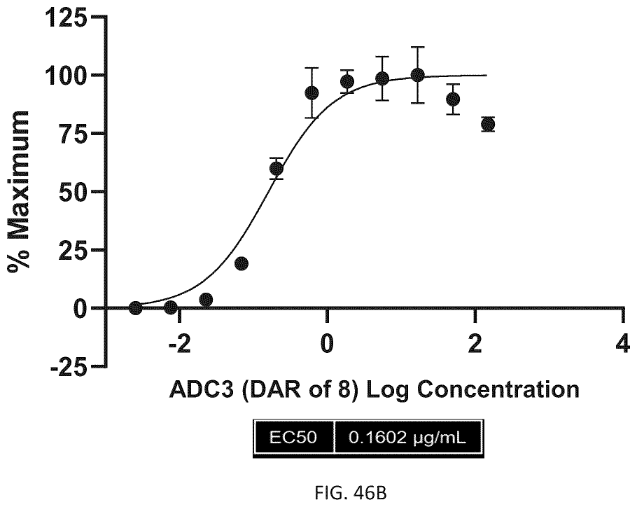

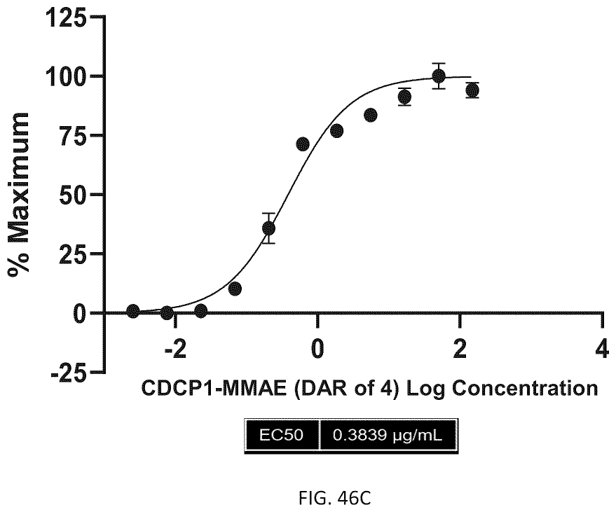

FIG. 46A is a graph of experimental data illustrating binding affinity of unconjugated CDCP1-mAbs to CDCP1+ cells. FIG. 46B is a graph of experimental data illustrating binding affinity of ADC3 (DAR of 8) to CDCP1+ cells. FIG. 46C is a graph of experimental data illustrating binding affinity of CDCP1-vcMMAE (DAR of 4) to CDCP1+ cells. The data shown in FIGS. 46A-46C demonstrates that unconjugated CDCP1-mAbs, ADC3 (DAR of 8), and CDCP1-vcMMAE (DAR of 4) bind comparably to CDCP1+ cells. The unconjugated CDCP1-mAbs, ADC3 (DAR of 8), and CDCP1-vcMMAE (DAR of 4) all have a binding affinity to CDCP1+ cells in the ~nM range. Conjugation of the CDCP1 antibody to the MMAE and compound 30 payloads did not alter binding affinity.

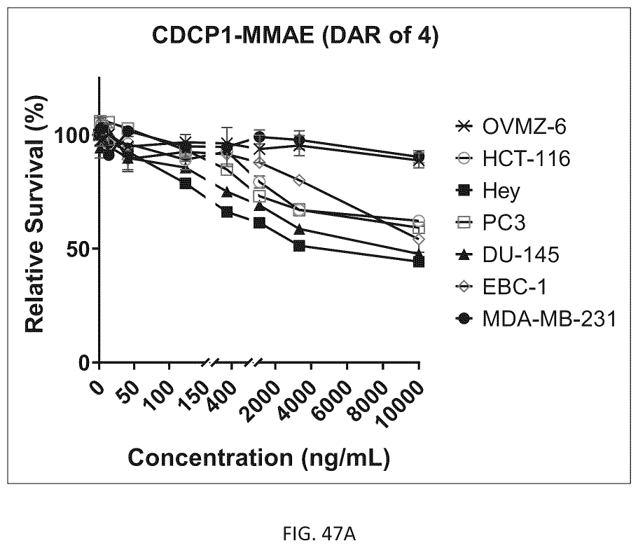

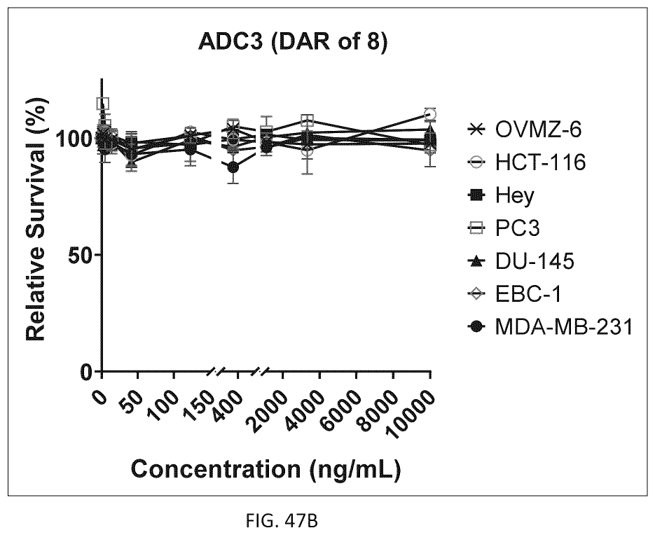

Preliminary in vitro cytotoxicity of CDCP1-vcMMAE (DAR of 4) and ADC3 (DAR of 8) is shown in FIGS. 47A and 47B. FIG. 47A is a graph of experimental data illustrating relative cell survival (%) versus concentration of CDCP1-vcMMAE (DAR of 4). FIG. 47B is a graph of experimental data illustrating relative cell survival (%) versus concentration of ADC3 (DAR of 8). The CDCP1-vcMMAE (DAR of 4) ADC showed modest in vitro cytotoxicity, with no activity in CDCP1 negative OVMZ-6 cells. The ADC3 (DAR of 8) showed minimal in vitro cytotoxicity.

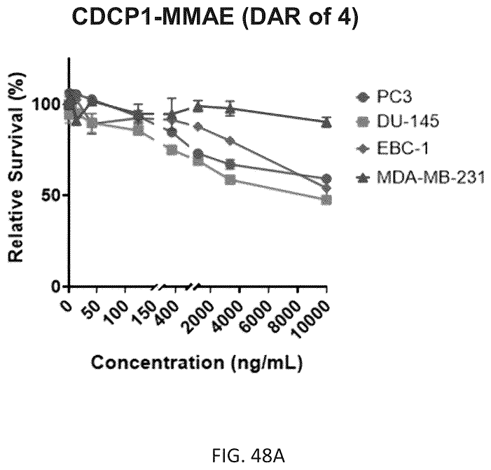

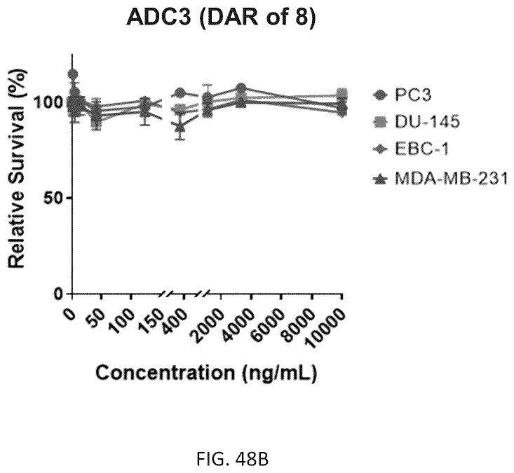

FIG. 48A is a graph of experimental data illustrating relative cell survival (%) versus concentration of CDCP1-vcMMAE (DAR of 4). FIG. 48B is a graph of experimental data illustrating relative cell survival (%) versus concentration of ADC3 (DAR of 8). Although not wishing to be bound by any particular theory, it is hypothesized that possible mechanisms for not observing in vitro cytotoxicity for ADC3 (DAR of 8) include: lack of ADC binding to cell surface; 3-5 day assay insufficient for cell killing given in vitro attainable intracellular exatecan exposures; antigen expression level is too low; inefficient internalization; insufficient exatecan release intracellularly; and/or lysosomal trapping of the payload. Studies to evaluate in vitro cytotoxicity are performed and include evaluating HEK-293 overexpression and benchmarking of T-Exa compared to T-DXd in vitro in N87 (HER2+++). ADC3 binding was confirmed, and in vitro killing using colony formation assay is assessed.

A non-limiting example of a benchmark study of T-Exa compared to T-DXd includes single dose administration of T-Exa and T-DXd at 5 mg/kg and 10 mg/kg, and multidose administration (e.g. Q7dx3): of T-Exa and T-DXd at 5 mg/kg and 10 mg/kg.

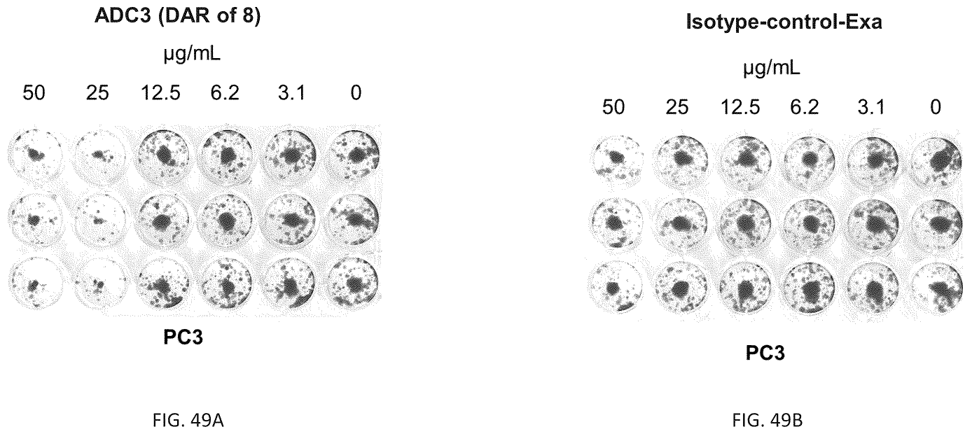

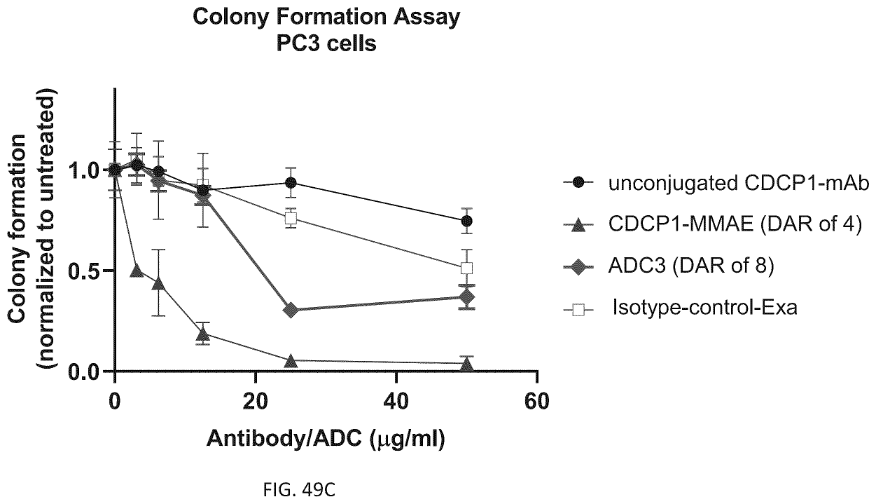

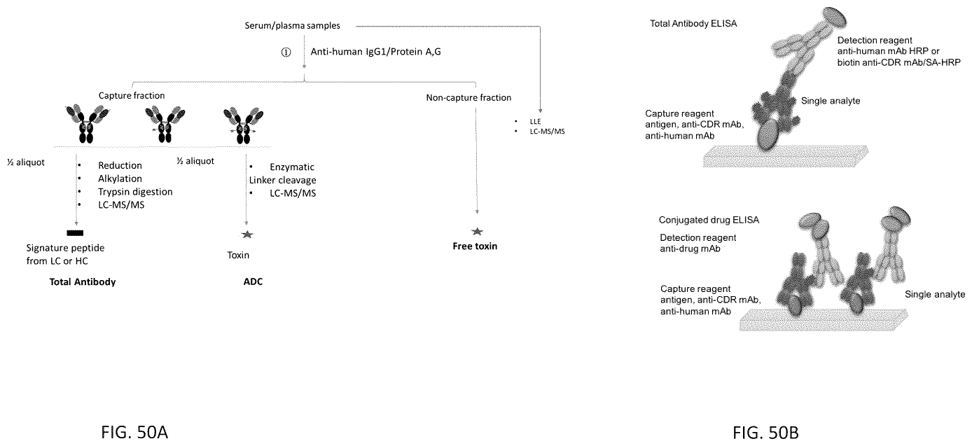

ADC3 (DAR of 8) demonstrated cytotoxicity in colony formation assays. FIG. 49A is an image of experimental data illustrating cytotoxicity of ADC3 (DAR of 8) in PC3 colony formation assay. FIG. 49B is an image of experimental data illustrating cytotoxicity of isotype-control exatecan in PC3 colony formation assay. FIG. 49C is a graph of experimental data illustrating colony formation (normalized to untreated colonies) versus concentration of unconjugated CDCP1-mAbs or ADC (μg/mL). The data shown in FIGS. 50A-50C indicate that ADC3 (DAR of 8) and isotype-control exatecan have CDCP1-specific activity.

FIGS. 50A-50B show non-limiting examples of pharmacokinetic strategies for evaluating antibody drug conjugates of the disclosure. FIG. 50A shows a non-limiting example of a LC-MS based analysis for ADCs, total mAb and payload (e.g. CDCP1-vcMMAE). FIG. 50B shows a non-limiting example of a ELISA-based analysis for ADC, total mAb, with LC-MS for payload (e.g. ADC3), CRL. LC-MS-based strategies have been well-established for MMAE. For the exatecan payload, total antibody (ELISA) and payload (LC-MS) methods were successfully developed for ADC3. However, anti-TOPO1 antibodies failed to detect exatecan for total ADC. While not wanting to be bound by any particular theory, it is hypothesized that a mAb specific to the exact exatecan structure may be useful, and an LC-MS approach is utilized.

### Example 7: Additional In Vitro and In Vivo Data for ADC3

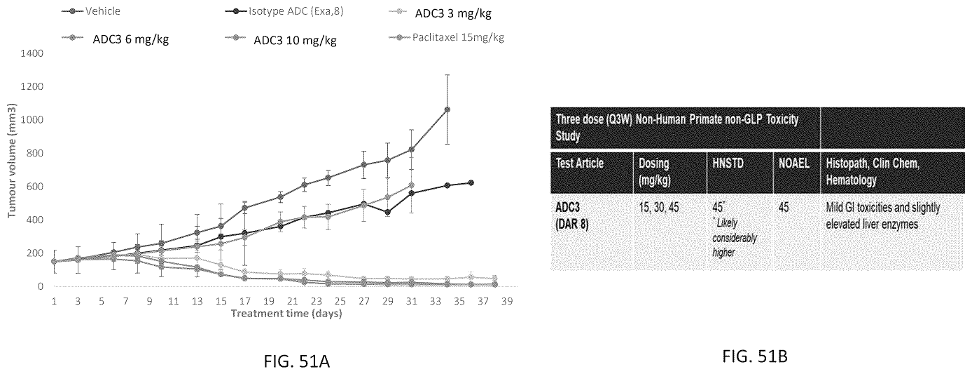

The efficacy and tolerability of ADC3 was found to be excellent (FIGS. 51A-51B). The toxicities of ADC3 are consistent with the exatecan payload mechanism of action, and no overt on-target toxicity was observed. Moreover, no lung issues or interstitial lung diseases (ILDs) were observed using ADC3. The free payload (exatecan) was not detected in cyno plasma (<0.0001 ug/ml). While not wishing to be bound by any particular theory, this result suggests that ADC3 has excellent stability. A MED of <3 mg/kg was observed.

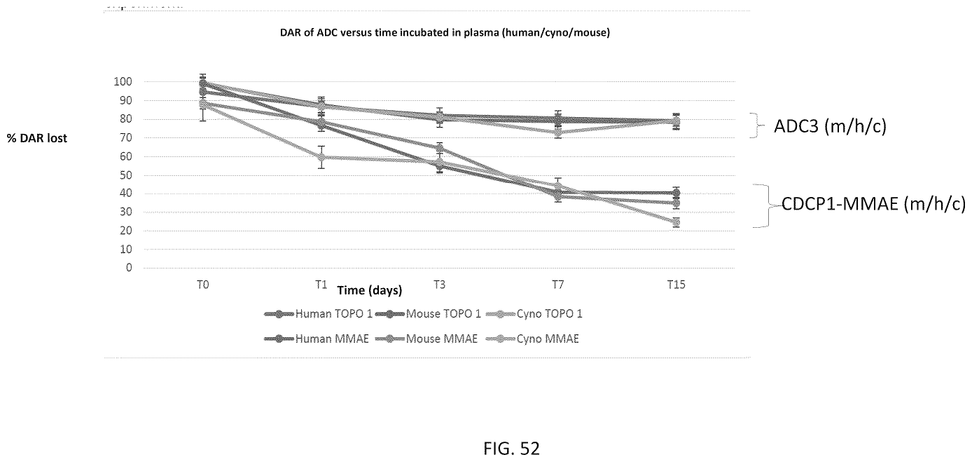

ADC3 was also found to be stable in mouse/human/cyno plasma, and significantly more stable than its equivalent MMAE-based ADC (CDCP1-MMAE) (FIG. 52). In plasma stability, ADC3 (DAR8) exhibited ~25% of linker-payload is lost after 7 days when incubated with mouse/human/cyno plasma; in contrast CDCP1-MMAE (DAR4) exhibited ~60-75% of linker-payload lost after 7 days when incubated with mouse/human/cyno plasma. ADC3 DAR of 8 became DAR6, whereas for CDCP1-MMAE, DARof 4 became DAR1. ADC3 also did not exhibit an apparent loss of free payload. While not wishing to be bound by any particular theory, this results may be due to the stability of the Val-Ala linkage. In contrast, free payload was observed with CDCP1-MMAE. While not wishing to be bound by any particular theory, this result may be due to the instability of the Val-Cit-PAB moiety of the CDCP1-MMAE ADC. While not wishing to be bound by any particular theory, in the case of both ADCs, retro-Michael (due to maleimide linkage) is hypothesized to be the predominant mechanism of linker-payload loss.

In a comparative repeat-dose rat toxicity study of Compound 30 versus Dxd (both DAR 8 on a Trastuzumab backbone), the ADCs were well tolerated after IV treatment once every 3 weeks for a total of two doses at either 20 or 60 mg/kg. Observed toxicities were predominantly related to the topoisomerase inhibition mechanism of action, and included effects on the hemopoietic system, evidenced by decreases in some red cell parameters. Significant changes were only observed in the T-Dxd cohorts, with negligible effects observed in the Compound 30 cohorts.

### Example 8: Additional In Vitro Data for Compound 30

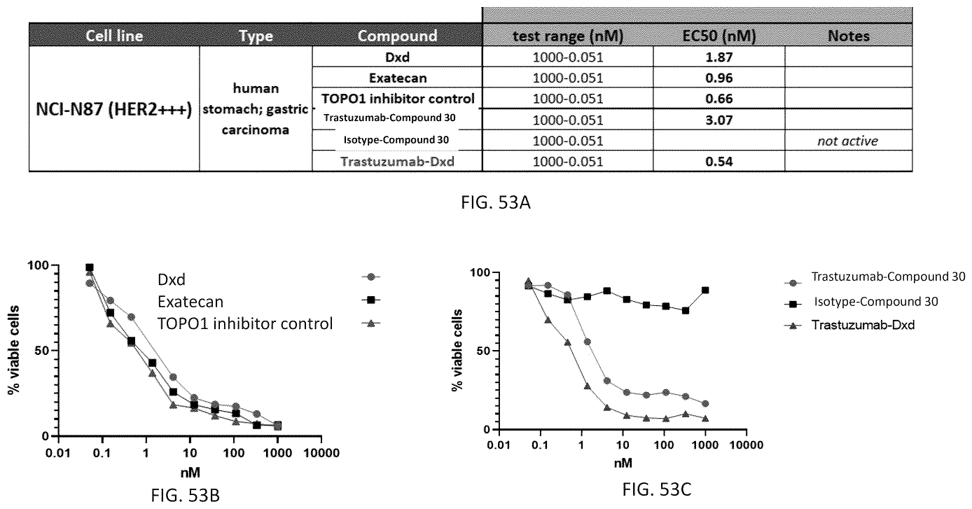

Despite free payloads being approximately equivalent in potency (FIG. 53B), trastuzumab-Compound 30 ADC was found to be less potent in HER2+++ line than T-Dxd ADC (FIG. 53A and FIG. 53C). Although not wishing to be bound by any particular theory, this result supports a slow cleavage mechanism of action for the Compound 30.

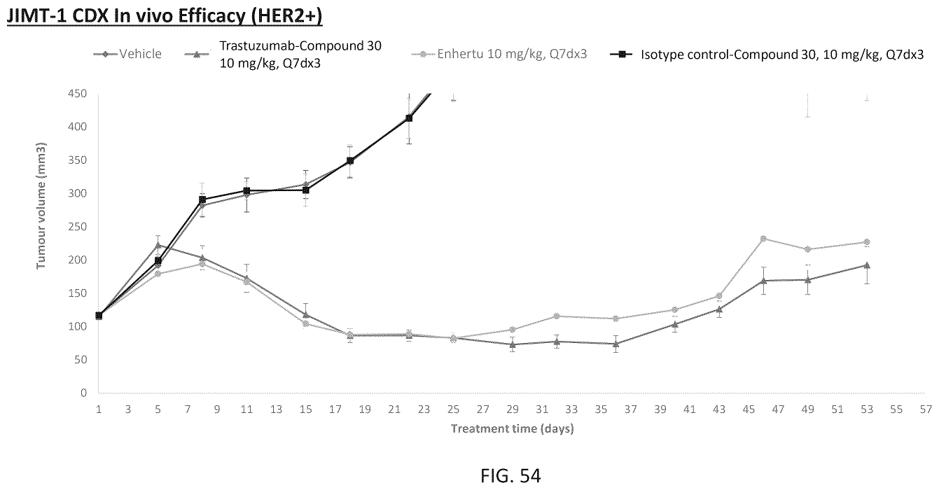

FIG. 54 illustrates experimental data demonstrating that in vivo efficacy shows more prolonged/sustained regressions with ADC3 compared to Enhertu®. JIMT-1 CDX in vivo efficacy (HER2+). While not wishing to be bound by any particular theory, this result supports a slower release mechanism of action for Compound 30. In a non-limiting example, levels of free payload are assessed in both trastuzumab-Compound 30 and trastuzumab-Dxd cohorts.

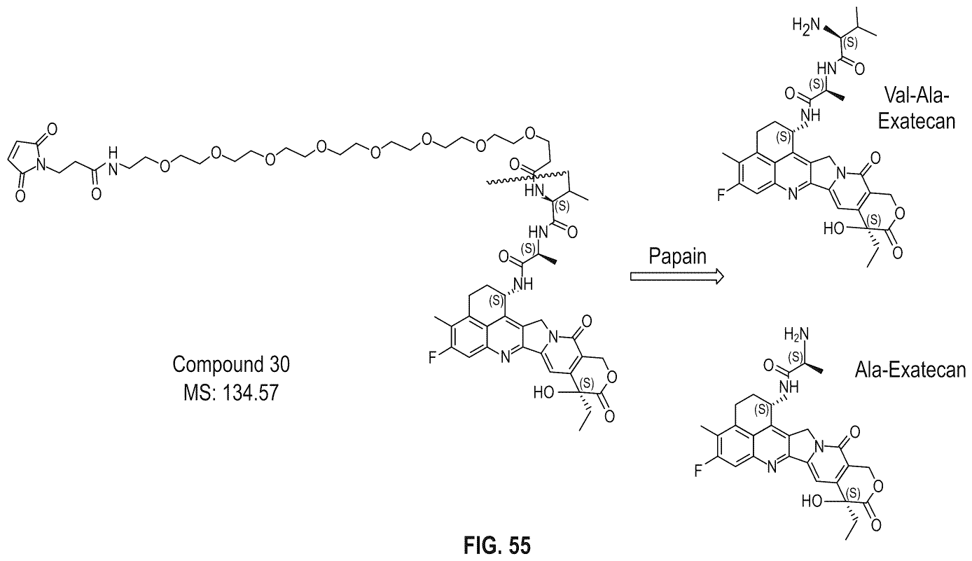

The mechanism of cleavage of the Val-Ala bond was expected to be via Cathepsin B, releasing Exatecan; however, when compounds 30 and ADC3 were treated with Papain (a Cathepsin B surrogate), the free payload was not liberated (FIG. 55). Instead, masses for Val-Ala-Exatecan and Ala-Exatecan were observed (Ala-Exatecan was confirmed to be an artefact of MS-based analysis). However, Val-Ala-Exatecan was found to be a true metabolite after reaction with Papain.

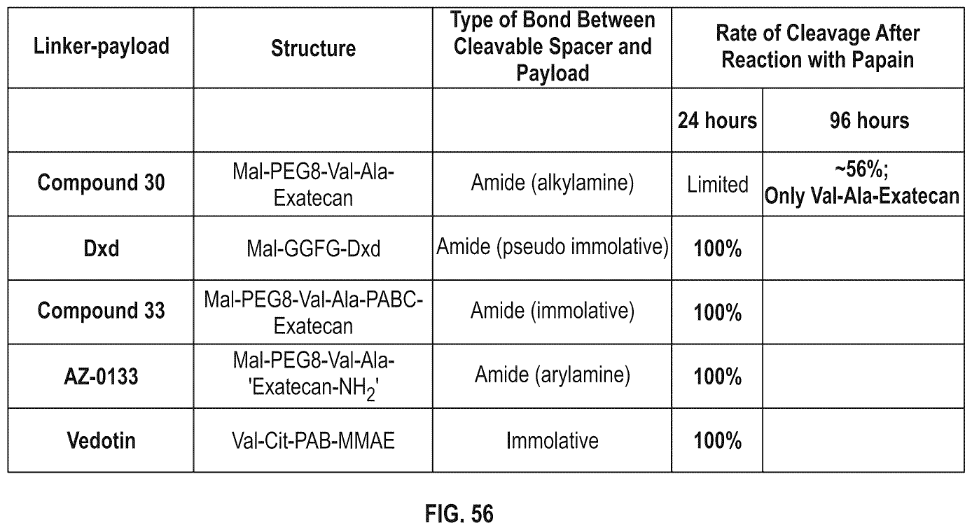

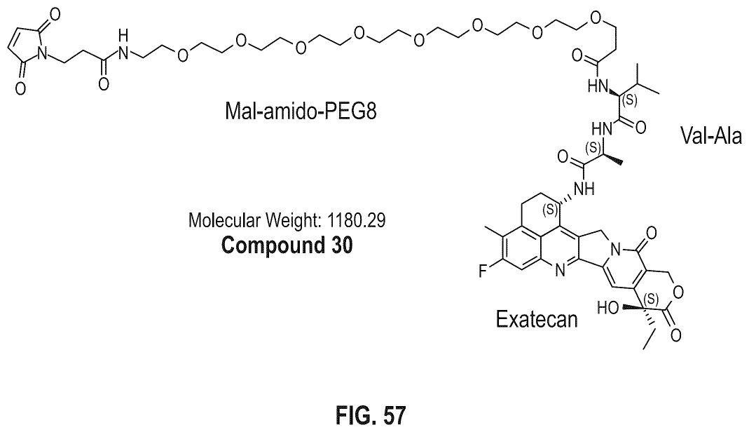

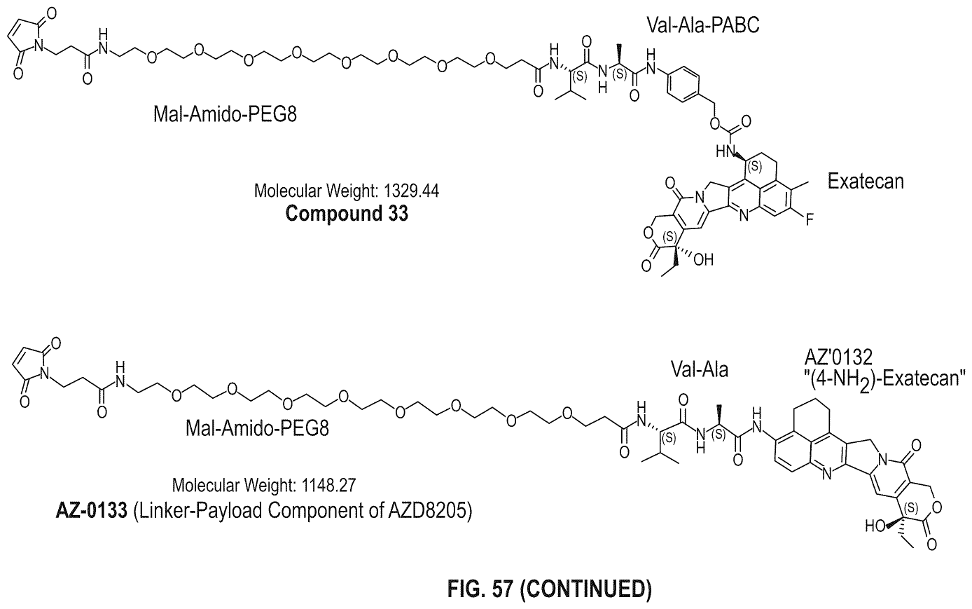

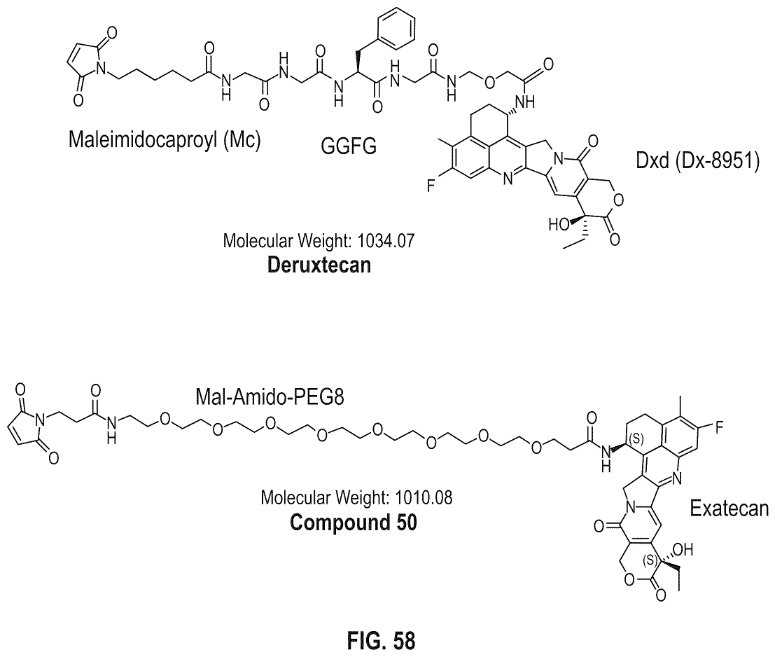

Compound 30 was found to cleave at a slower rate compared to other TOPO1 inhibitors having Cathepsin B cleavage mechanisms (FIG. 56). Compound 30 was found to cleaves at a much slower rate than other TOPO1-based linker/payloads (e.g. deruxtecan-FIG. 57) and other Val-Ala-containing linker-payloads (e.g. Compound 33, AZ-0133 (FIG. 58). While not wishing to be bound by any particular theory, since premature cleavage is associated with enhanced toxicities, the lack of toxicity observed for ADC3 in the non-GLP cyno study may be due in part to the enhanced stability of Compound 30. While not wishing to be bound by any particular theory, it is hypothesized that the alkylamide bond between Ala and exatecan in Compound 30 is inherently more stable than the arylamide bond between Ala and exatecan (e.g. AZ-1033), rendering the alkylamide bond less prone to cleavage.

### Example 9: S9 Liver Microsome Studies

A series of TOPO1 inhibitor-containing ADCs were generated at DAR8 using a Trastuzumab backbone. The linker-payload on each ADC was varied through modification of the protease cleavable spacer (e.g., alkylamide link to exatecan versus arylamide link to exatecan, GGFG versus dipeptide spacer) or mechanism of release (e.g., with and without cleavable and immolative spacer).

Payload-containing catabolite formation of six antibody-drug conjugates namely Trastuzumab-Compound 30, Trastuzumab-Compound 33, Trastuzumab-Compound 50, Trastuzumab-AZ′0133, Trastuzumab-Deruxtecan, and Trastuzumab-Emtansine, were studied using human and monkey liver S9 fractions in mildly acidic conditions over the time period of 24 h, with initial test concentration of 5 μM. Samples were analysed using LC/QE-orbitrap-MS.

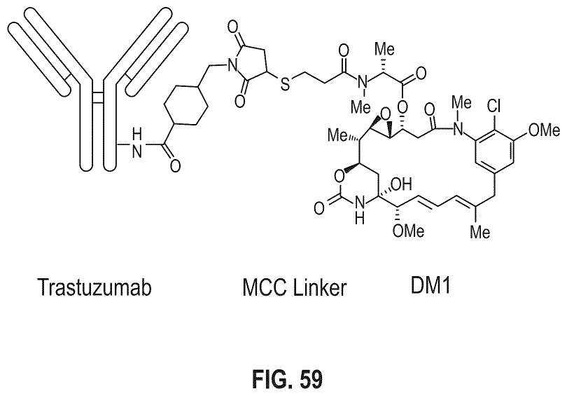

In the case of each cleavable spacer, the liberated construct in both human and cyno S9 was shown to be the TOPO1 inhibitor (i.e., exatecan or Dxd). T-DM1 was included as a control (FIG. 59), and despite being classified as a non-cleavable linker, DM1 (cytotoxin) was observed as a released agent. Similarly, an exatecan derivative that did not contain a dipeptide spacer (and was therefore classed as non-cleavable; Compound 50 (FIG. 58)) also released small amounts of exatecan in both human and cyno S9 at longer timepoints (e.g., 24 hours). See Table 4 below.

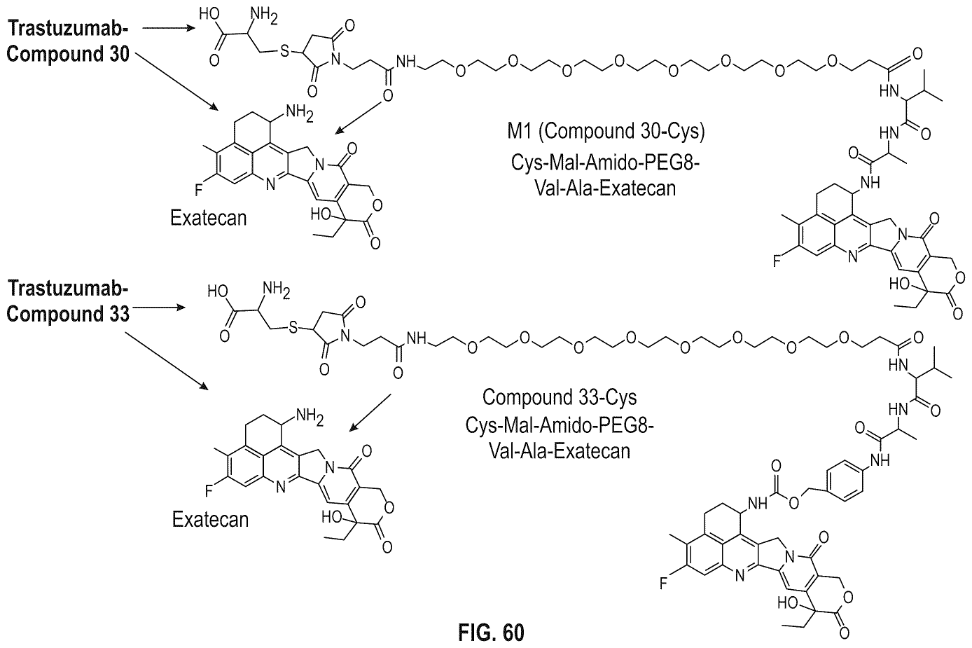

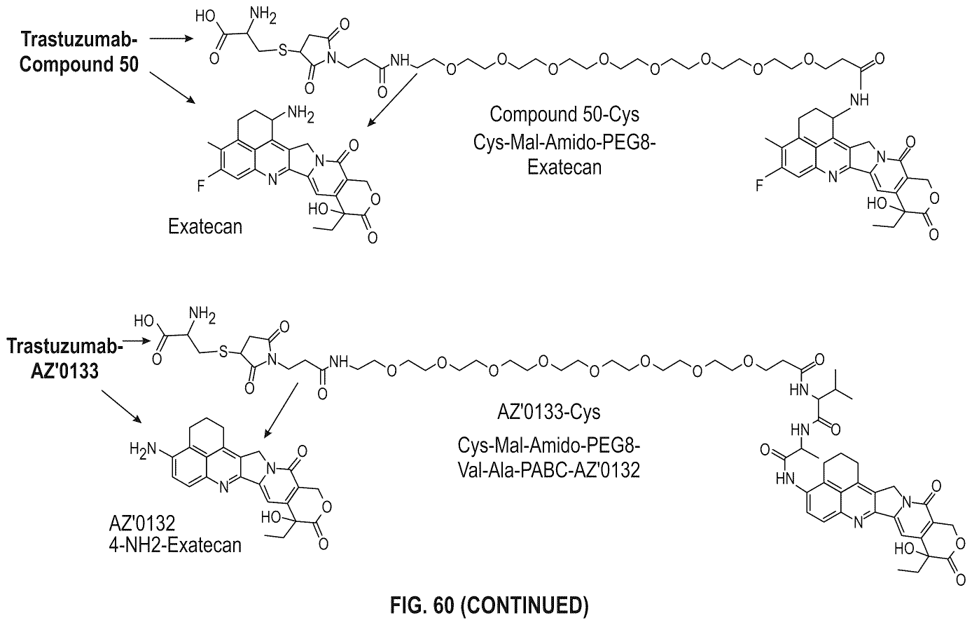

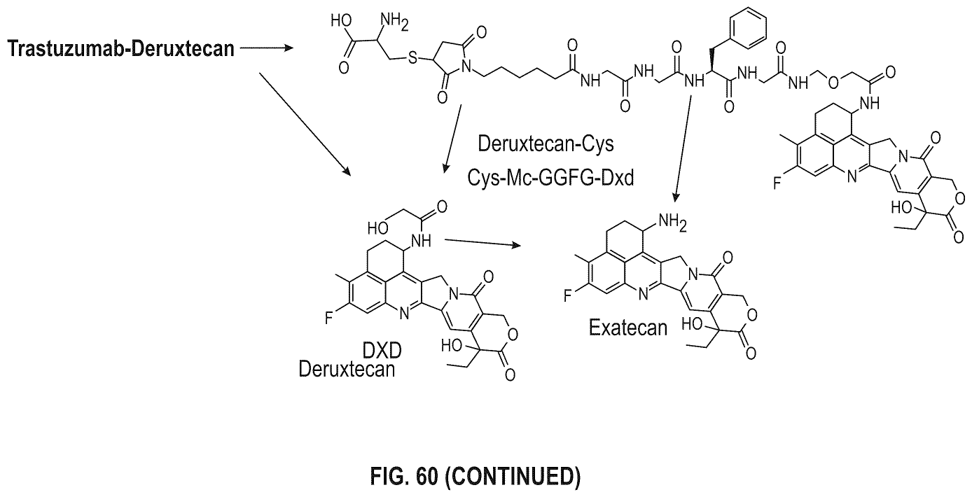

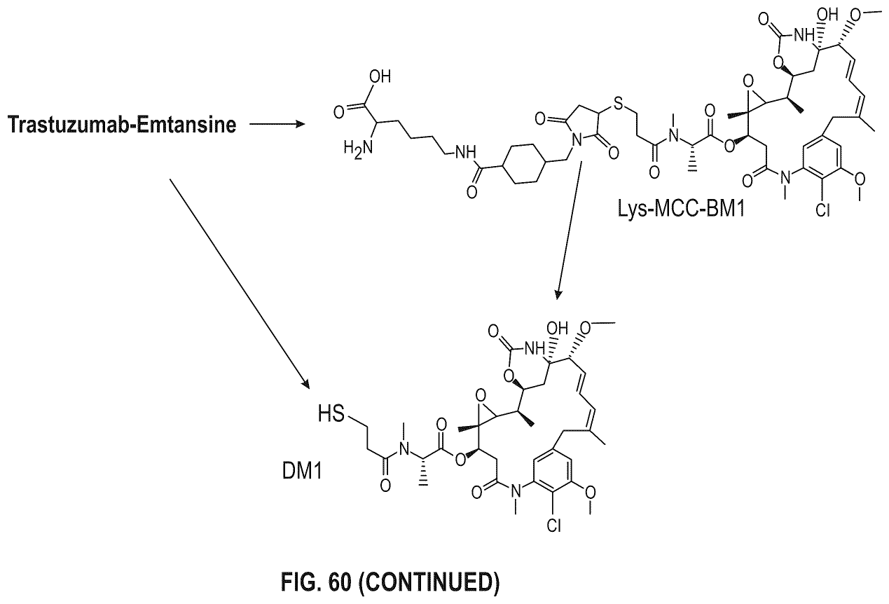

In total, formation of four payload-catabolites and six payload-linker-amino acid catabolites were followed. These were Exatecan and Cys-Compound 30 (M1) for Trastuzumab-Compound 30, Exatecan and Cys-mal-amido-peg8-val-ala-PABC-Exatecan for Trastuzumab-Compound 33, Exatecan and Cys-mal-amido-PEG8-exatecan for Trastuzumab-Compound 50, 4NH2-Exatecan and Cys-mal-amido-PEG8-Val-Ala-PABC-AZ′0132 for Trastuzumab-AZ′0133, DXd and Cys-Mc-GGFG-DXd for Trastuzumab-Deruxtecan, and DM1 and Lys-MCC-DM1 for Trastuzumab-Emtansine. The amount of released payloads were estimated by comparing to 1 μM reference compounds. FIG. 60 shows the structures of the catabolites that were followed.

In general, the release of payload and payload-linker-amino acid catabolites were much higher in incubations with human liver S9 fractions than with monkey liver S9 fractions (more than 10-fold) and the highest levels were observed at 24 hour time point.

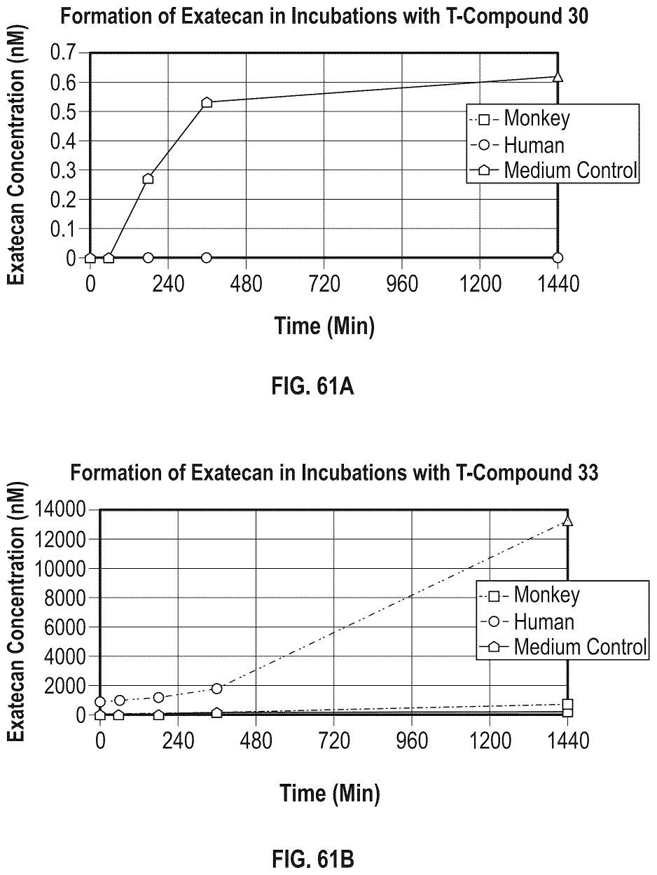

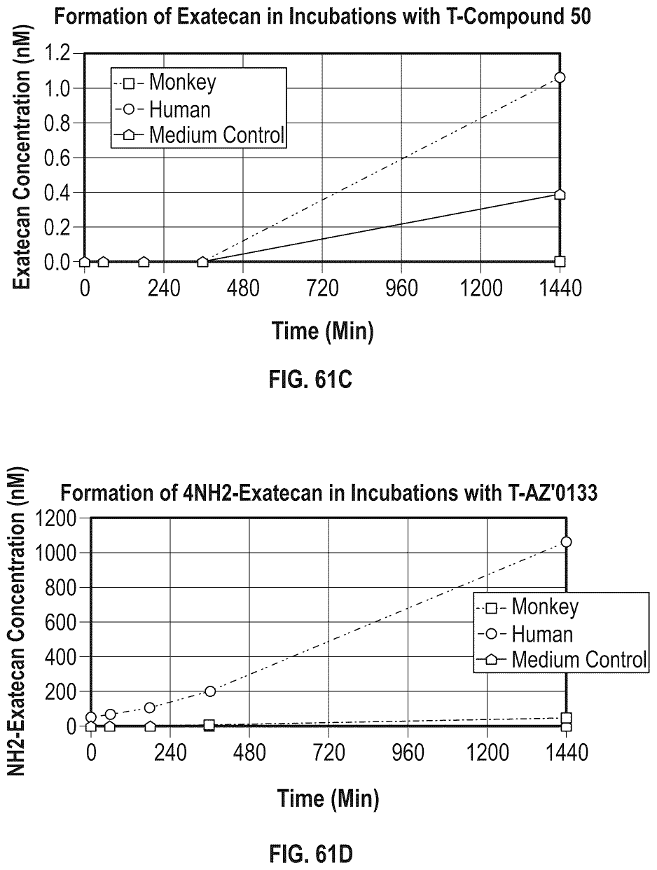

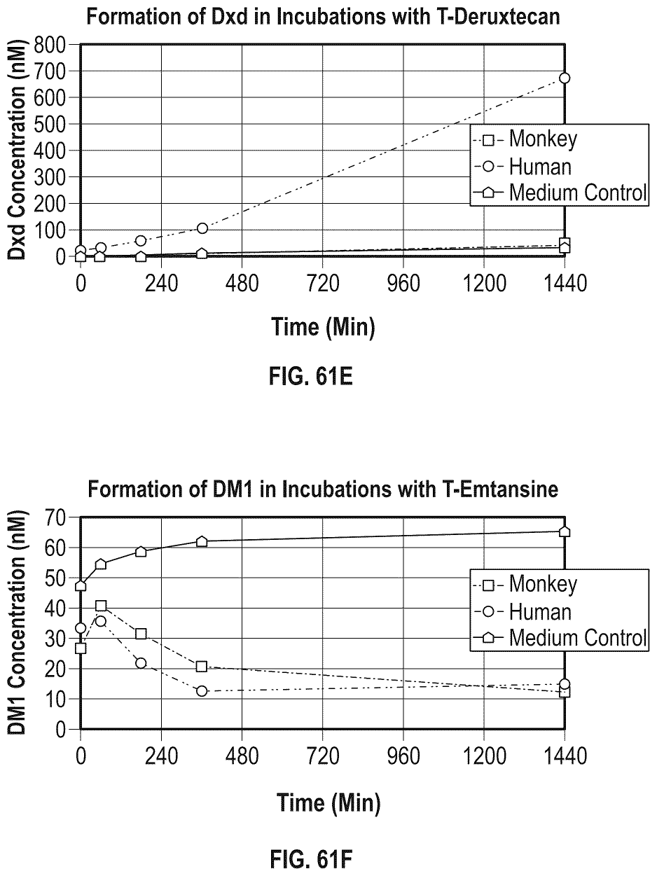

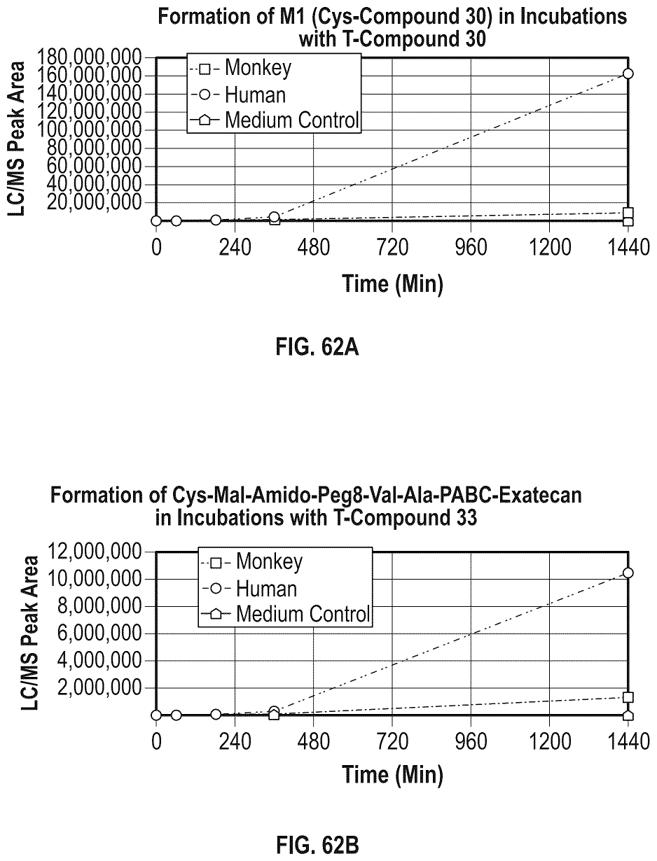

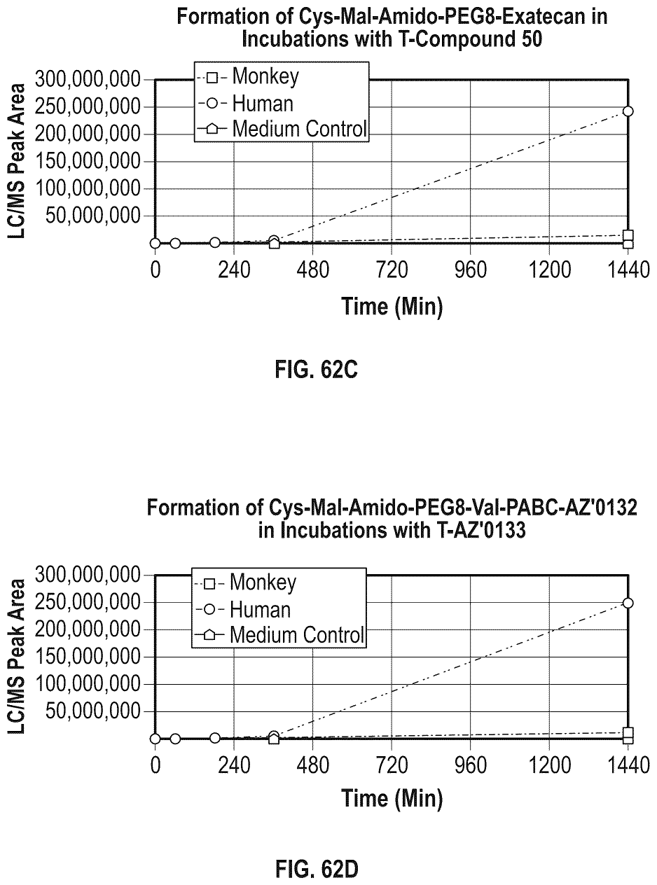

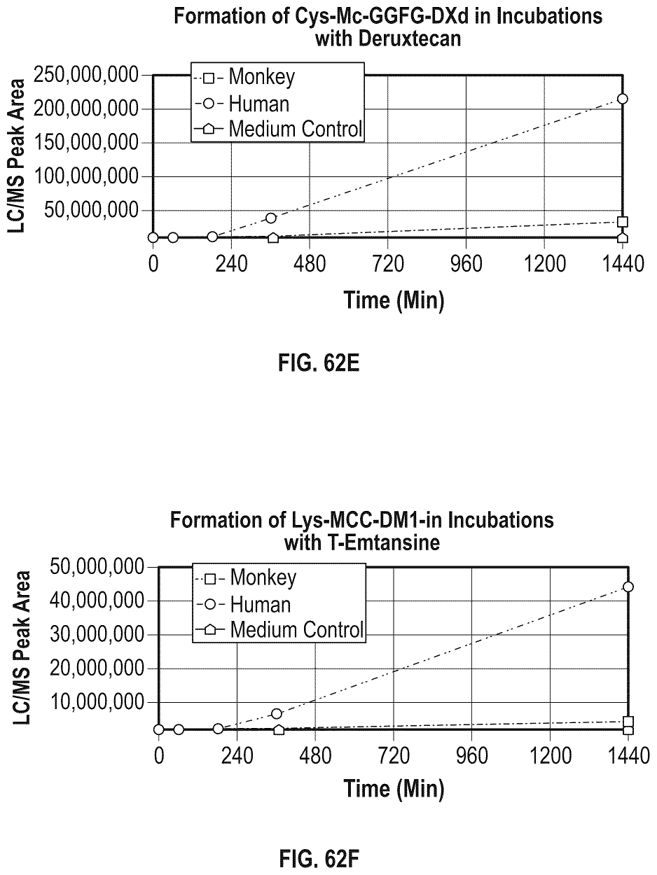

In incubations with Trastuzumab-Compound 30, no exatecan was released either in human or monkey liver S9 fractions. Minor amounts of Exatecan (<1 nM), was only formed in control incubations in media. The control incubations include each ADC without the addition of S9. FIG. 61A shows that the concentration of exatecan in monkey and human liver S9 incubations was not observed, while the concentration of exatecan formed in the medium control was not identified above 0.7 nM after 1440 minutes. While not necessarily wanting to be bound by any particular theory, the identification of exatecan in the control incubation was hypothesized to be due to an artifact. Instead, high levels of cys-Compound 30 (M1) was formed in human liver S9 incubations, while in incubations with monkey liver S9 fractions much lower levels of cys-Compound 30 (M1) was formed when compared to human S9 (ca 5%). As shown in FIG. 62A, the LC/MS peak area for the identification of cys-Compound 30 (M1) was around 160 MM after 1440 minutes, compared to 10 MM for trastuzumab-Compound 33.

High concentrations of exatecan was formed in incubations with Trastuzumab-Compound 33 and the highest amount was formed in human liver S9 (13 μM at 24 h time point). Only about 0.8 μM was formed in incubations with monkey liver S9 at 24 h time point. Exatecan was also formed in control incubation in buffer with about 0.3 μM amount at 24 h time point). The payload-linker-amino-acid (Cys-mal-amido-peg8-val-ala-PABC-Exatecan) of Trastuzumab-Compound 33 was formed more in human than in monkey liver S9 incubations, but in both, only with low levels.

In incubations with Trastuzumab-Compound 50, only trace levels of the payload exatecan was released (less than 1.1 nM in human S9 at 24 h time point), while high levels of Cys-Mal-amido-PEG8-exatecan was formed in human liver S9 incubations and lower levels in incubations with monkey liver S9 (ca. 6%).

In incubations with Trastuzumab-AZ′0133, at 24-hour time point, estimated 1000 nM amount of payload (4NH2-exatecan) was released with human liver S9 fraction, while much lower level (ca. 55 nM) of payload was released with monkey liver S9 fraction. Similarly, high levels of payload-linker-amino acid (Cys-mal-amido-PEG8-Val-Ala-PABC-AZ′0132) was also released in incubations with human liver S9 fraction, and much lower levels (ca. 4% of what released in human) in incubations with monkey liver S9 fraction. Small amounts of both payload and payload-linker-amino-acid for Trastuzumab-AZ-0133 was also released in control incubations with media.

In incubations with Trastuzumab-Deruxtecan, estimated 670 nM amount of payload deruxtecan (DXd) was released with human liver S9 fraction. Only low amounts of DXd was released both in monkey liver S9 fraction and medium control incubation (45 nM and 38 nM at 24 h time point, respectively). High levels of payload-linker containing cysteine-residue (Cys-Mc-GGFG-DXd) was released in human liver S9 fraction and about ten-fold lower levels in monkey liver S9 fraction. Low levels of exatecan were also detected, with 2.6 nM in monkey, 2.0 nM in human and 5.1 nM in buffer incubation at 24 h time point. While not wishing to be bound by any particular theory, it is hypothesized that exatecan can be formed from deruxtecan via hydrolysis.

In incubations with Trastuzumab-Emtansine, low levels of payload DM1 was observed at all time points and with all incubation conditions, while constant release of payload-linker containing lysine-residue (Lys-MCC-DM1) was observed throughout the incubation time period in human liver S9 fraction

The Compound 30-containing construct was differentiated as any released moiety was found to contain the linker-payload Compound 30. Free exatecan was not observed. This differs from traditional cleavable linker-payloads, but also traditional non-cleavable linker-payloads (e.g., DM1).

TABLE 4
S9 Liver Microsome Data

| | Cleavable or | Detected Metabolites in Liver |
|---|---|---|
| ADC Construct | Non-Cleavable? | Microsome S9 Studies |
| Trastuzumab-Mal-GGFG-Dxd | Cleavable | Dxd and Exatecan |
| (Enhertu ®) (FIG. 58) | | |
| Trastuzumab-DM1 (Kadcyla ®) | Non-Cleavable | DM1 |
| (FIG. 59) | | |
| Trastuzumab-Compound 30 | Cleavable (alkylamide) | Cys-Mal-PEG8-Val-Ala-Exatecan- |
| | | containing adduct |
| Trastuzuma--Compound 33 | Cleavable (with immolative | Exatecan |
| | linker) | |
| Trastuzumab-Compound 50 | Non-cleavable | Exatecan |
| Trastuzumab-AZ-0133 (FIG. 57) | Cleavable (arylamide) | Exatecan |

TABLE 5
UPLC/ESI/QE-orbitrap-MS data obtained for the followed catabolites

| Trastuzumab-Compound 30 | | | | | | |
|---|---|---|---|---|---|---|
| Exatecan | 3.92 | C24H22FN3O4 | [M + H]+ | 436.1667 | 436.1667 | 435.1594 |
| Cys-PHN23-22 = M1 | 5.93 | C61H85FN8O20S | [M + 2H]2+ | 651.2863 | 651.2865 | 1300.5576 |
| Trastuzumab-Compound 33 | | | | | | |
| Exatecan | 3.92 | C24H22FN3O4 | [M + H]+ | 436.1667 | 436.1667 | 435.1594 |
| Cys-mal-amido-peg8-val-ala-PABC- | 6.33 | C69H92FN9O22S | [M + 2H]2+ | 725.8115 | 725.8104 | 1449.6062 |
| Exatecan | | | | | | |
| Trastuzumab-Compound 50 | | | | | | |
| Exatecan | 3.92 | C24H22FN3O4 | [M + H]+ | 436.1667 | 436.1667 | 435.1594 |
| Cys-Mal-amido-PEG8-exatecan | 5.64 | C53H71FN6O18S | [M + 2H]2+ | 566.2338 | 566.2338 | 1130.4530 |
| Trastuzumab-AZ′0133 | | | | | | |
| 4NH2-Exatecan | 5.12 | C23H21N3O4 | [M + H]+ | 404.1602 | 404.1605 | 403.1532 |
| Cys-mal-amido-PEG8-Val-Ala-PABC- | 5.40 | C60H84N8O20S | [M + 2H]2+ | 635.2830 | 635.2834 | 1268.5523 |
| AZ′0132 | | | | | | |
| Trastuzumab-Deruxtecan | | | | | | |
| Dxd | 5.41 | C26H24FN3O6 | [M + H]+ | 494.1721 | 494.1722 | 493.1649 |
| Cys-Mc-GGFG-DXd | 5.80 | C55H63FN10O15S | [M + 2H]2+ | 578.2157 | 578.2162 | 1154.4179 |
| Trastuzumab-Emtansine | | | | | | |
| DM1 | 6.61 | C35H48ClN3O10S | [M + Na]+ | 760.2643 | 760.2641 | 737.2749 |
| Lys-MCC-DM1 | 6.02 | C53H75ClN6O15S | [M + 2H]2+ | 552.2423 | 552.2423 | 1102.4700 |

### Materials and Methods

The UPLC/QE-orbitrap-MS data obtained for the detected catabolites is presented in Table 5. All catabolites were tentatively identified using accurate mass data and identifications are shown in Table 5 (FIG. 60 shows the structures of catabolites that were followed). The amount of the released payloads exatecan, 4-NH2-exatecan, DXd, and DM1 were estimated based on comparison to 1 μM reference compounds and the results (nM) are shown in Table 6 and FIG. 61. The peak areas for the reference payloads and payloads are shown in Table 8 and 9. The catabolic profiles expressed as peak areas for the detected payload-linker-amino-acids (cysteine or lysine) are shown in Table 7 and FIG. 62.

TABLE 6
The concentration of released payloads in different incubations (nM)

| | | | | | | 4NH2- | | |
|---|---|---|---|---|---|---|---|---|
| | | | Exatecan | Exatecan | Exatecan | Exatecan | DXd | DM1 |
| | | | [nM] in T- | [nM] in T- | [nM] in T- | [nM] in T- | [nM] in T- | [nM] in T- |
| | | | Compound 30 | Compound 33 | Compound 50 | AZ′0133 | Deruxtecan | Emtansine |
| Monkey | 0 | min | — | 157 | — | 4 | 3 | 27 |
| S9 | 60 | min | — | 153 | — | 5 | 5 | 41 |
| | 180 | min | — | 159 | — | 5 | 6 | 32 |
| | 360 | min | — | 218 | — | 14 | 10 | 21 |
| | 1440 | min | — | 776 | — | 55 | 45 | 12 |
| Human | 0 | min | — | 963 | — | 58 | 26 | 34 |
| S9 | 60 | min | — | 1,059 | — | 77 | 37 | 37 |
| | 180 | min | — | 1,252 | — | 115 | 63 | 22 |
| | 360 | min | — | 1,849 | — | 205 | 108 | 13 |
| | 1440 | min | — | 13,059 | 1.1 | 1042 | 674 | 15 |
| Buffer | 0 | min | — | 30 | — | — | 2 | 48 |
| without | 60 | min | — | 67 | — | — | 6 | 55 |
| S9 | 180 | min | 0.3 | 136 | — | 3 | 9 | 59 |
| | 360 | min | 0.5 | 256 | — | 7 | 16 | 63 |
| | 1440 | min | 0.6 | 316 | 0.4 | 8 | 38 | 66 |

TABLE 7
Payload-linker-amino acid peak areas for different incubations.

| | | | | Cys-mal- | | | | |
|---|---|---|---|---|---|---|---|---|
| | | | | amido- | Cys-Mal- | Cys-mal- | | |
| | | | | peg8-val- | amido- | amido- | | |
| | | | M1 (PHN23- | ala-PABC- | PEG8- | PEG8-Val- | Cys-Mc- | Lys-MCC- |
| | | | 22-cys) | Exatecan | exatecan | Ala-PABC- | GGFG-DXd | DM1 |
| | | | in T- | in T- | in T- | AZ′0132 in | in T- | in T- |
| | | | Compound 30 | Compound 33 | Compound 50 | T-AZ′0133 | Deruxtecan | Emtansine |
| Monkey | 0 | min | — | — | — | — | — | — |
| S9 | 60 | min | — | — | 57,575 | — | — | — |
| | 180 | min | 563,516 | — | 246,925 | 102,198 | 109,943 | — |
| | 360 | min | 669,120 | — | 998,265 | 258,625 | 536,289 | — |
| | 1440 | min | 8,771,950 | 1,369,276 | 14,754,916 | 9,847,683 | 24,006,409 | 2,428,809 |
| Human | 0 | min | — | — | 134,763 | 99,803 | — | — |
| S9 | 60 | min | 397,081 | — | 220,977 | 71,519 | 167,461 | — |
| | 180 | min | 801,114 | — | 176,682 | 321,565 | 4,491,677 | 517,122 |
| | 360 | min | 3,859,221 | 222,034 | 7,089,001 | 4,221,293 | 30,429,557 | 4,442,635 |
| | 1440 | min | 161,514,461 | 10,404,502 | 243,736,565 | 247,679,006 | 210,850,461 | 44,337,978 |
| Buffer | 0 | min | — | — | — | — | — | — |
| without | 60 | min | — | — | — | — | — | — |
| S9 | 180 | min | — | — | — | — | — | — |
| | 360 | min | — | — | — | — | — | — |
| | 1440 | min | — | — | — | — | — | — |

TABLE 8
LC/MS peak areas for 1000 nM reference payloads

| Reference | 1000 nM | 1000 nM 4NH2- | 1000 nM | 1000 nM |
|---|---|---|---|---|
| payload | Exatecan | Exatecan | DXd | DM1 |
| LC/MS | 96,188,009 | 58,555,118 | 61,405,961 | 29,880,192 |
| peak area | | | | |

TABLE 9
LC/MS peak areas for released payloads in different incubations

| | | | | | | 4NH2- | | |
|---|---|---|---|---|---|---|---|---|
| | | | | | | Exatecan | | |
| | | | Exatecan in | Exatecan in | Exatecan in | in T- | DXd in T- | DM1 in T- |
| | | | Compound 30 | Acompound 33 | Compound 55 | AZ′0133 | Deruxtecan | Emtansine |
| Monkey | 0 | min | — | 15,104,561 | — | 220,160 | 199,571 | 810,008 |
| S9 | 60 | min | — | 14,669,372 | — | 294,076 | 307,593 | 1,232,044 |
| | 180 | min | — | 15,275,005 | — | 292,508 | 341,421 | 947,857 |
| | 360 | min | — | 21,002,687 | — | 818,342 | 596,372 | 624,956 |
| | 1440 | min | — | 74,672,125 | — | 3,223,383 | 2,736,070 | 372,665 |
| Human | 0 | min | — | 92,635,746 | — | 3,394,482 | 1,618,214 | 1,013,482 |
| S9 | 60 | min | — | 101,910,113 | — | 4,487,737 | 2,258,738 | 1,092,822 |
| | 180 | min | — | 120,401,385 | — | 6,716,960 | 3,876,987 | 661,127 |
| | 360 | min | — | 177,812,760 | — | 12,025,932 | 6,638,596 | 380,572 |
| | 1440 | min | — | 1,256,123,343 | 102,237 | 61,034,393 | 41,383,290 | 441,270 |
| Buffer | 0 | min | — | 2,913,338 | — | — | 147,119 | 1,431,764 |
| without | 60 | min | — | 6,462,443 | — | — | 384,075 | 1,641,380 |
| S9 | 180 | min | 26,179 | 13,090,877 | — | 172,299 | 550,232 | 1,765,527 |
| | 360 | min | 51,267 | 24,601,000 | — | 428,067 | 967,374 | 1,869,809 |
| | 1440 | min | 59,452 | 30,350,534 | 36,832 | 497,061 | 2,307,156 | 1,958,643 |

### Chemicals and Suppliers.

HPLC grade methanol and acetonitrile: Merck (Darmstadt, Germany). HPLC grade formic acid, acetic acid and ammonium formate: BDH Laboratory Supplies (Poole, UK) Other chemicals: Sigma Aldrich (Helsinki, Finland), the highest purity available. Water was in-house freshly prepared with a Direct-Q3 (Millipore Oy, Espoo, Finland) purification system and UP grade (ultra pure, 18.2 MΩ).

The study compounds Trastuzumab-Compound 30, Trastuzumab-Compound 33, Trastuzumab-Compound 50, Trastuzumab-AZ′0133, Trastuzumab-Deruxtecan, and Trastuzumab-Emtansine were obtained in buffer and the reference compounds exatecan mesylate (10 mM), Dxd (1 mM), DM1 (1 mM) and 4NH2-Exatecan (1 mM) were obtained as DMSO stock solutions. Table 10 shows incubation materials & procedures in liver S9, pH 5.0.

TABLE 10
Incubation materials & procedures in liver S9, pH 5.0

| | |
|---|---|
| Enzyme source/ | Pooled liver S9 |
| supplier | fraction/ |
| | Bioreclamation |
| | IVT and |
| | Xenotech) |
| | See Appendix I |
| | for detailed |
| | descriptions |
| Species/strain | Cynomolgus |
| | monkey (LOT |
| | KPQ, male) |
| | Human (LOT |
| | 1610015, mixed |
| | gender) |

Conditions in the final incubation:

| | |
|---|---|
| Time points | 0, 1, 3, 6, 24 h ± S9 |
| Concentration | 5 μM |
| Protein content | 1.5 mg/ml |
| Replicates | 1 |
| Buffer | 50 mM Na-acetate, |
| | pH 5.0 |
| Cofactors | No cofactors |
| Shaking | 600 rpm |
| Temperature | 37° C. |
| Incubation | 300 μl |
| volume | |
| Sampling volume | 40 μl |
| Spiking | Trastuzumab- |
| | Compound 30: |
| | 31.5 μl of 7.36 |
| | mg/ml (47.6 μM) |
| | Trastuzumab- |
| | Compound 33: |
| | 40.0 μl of 5.84 |
| | m/ml (37.5 μM) |
| | Trastuzumab- |
| | Compound 50: |
| | 38.6 μl of 5.96 |
| | mg/ml (38.9 μM) |
| | Trastuzumab- |
| | AZ′0133: 37.4 μl |
| | of 6.19 mg/ml |
| | (40.1 μM) |
| | Trastuzumab- |
| | Deruxtecan: |
| | 51.1 μl of 4.51 |
| | mg/ml (29.3 μM) |
| | Trastuzumab- |
| | Emtansine: 15 μl |
| | of 14.85 mg/ml |
| | (99.8 μM) |
| Quenching | 2-fold volume |
| solvent | of 75% ACN |
| | (with 100 nM |
| | phenacetin) |
| Storage | −20° C. until |
| | analysis |
| Controls | Blank |
| | incubation |
| | without study |
| | compound |
| Reference | Exatecan (1 μM) |
| compounds: | 4-NH2-Exatecan |
| | (1 μM) |
| | Dxd (1 μM) |
| | DM1 (1 μM) |

A number of patent and non-patent publications are cited herein in order to describe the state of the art to which this disclosure pertains. The entire disclosure of each of these publications is incorporated by reference herein.

While certain embodiments of the present disclosure have been described and/or exemplified above, various other embodiments will be apparent to those skilled in the art from the foregoing disclosure. The present disclosure is, therefore, not limited to the particular embodiments described and/or exemplified, but is capable of considerable variation and modification without departure from the scope and spirit of the appended claims.

Moreover, as used herein, the term “about” means that amounts, sizes, formulations, parameters, shapes and other quantities and characteristics are not and need not be exact, but may be approximate and/or larger or smaller, as desired, reflecting tolerances, conversion factors, rounding off, measurement error and the like, and other factors known to those of skill in the art. In general, an amount, size, formulation, parameter, shape or other quantity or characteristic is “about” or “approximate” whether or not expressly stated to be such.

Furthermore, the transitional terms “comprising”, “consisting essentially of” and “consisting of”, when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinary associated with the specified material(s). The term “consisting essentially of” limits the scope of a claim to the specified elements, steps or material(s) and those that do not materially affect the basic and novel characteristic(s) of the claimed disclosure. All compounds, compositions, formulations, and methods described herein that embody the present disclosure can, in alternate embodiments, be more specifically defined by any of the transitional terms “comprising,” “consisting essentially of,” and “consisting of.”

1. An antibody-drug conjugate having formula (I): 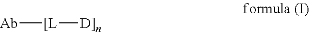
wherein in formula (I):
Ab comprises an antibody or binding fragment thereof, wherein the antibody or binding fragment thereof specifically binds CUB Domain-Containing Protein-1 (CDCP1);
L is a linker of the formula —R*-L₁-L_{A}-;
R* is succinimide;
L₁ is —[CH₂]₁₋₃—C(O)NH—;
L_{A} is —[CH₂CH₂O]ₚ—(CH₂)₁₋₅—C(O)—X_{AA}—, wherein p is an integer from 5 to 10, and X_{AA} is an amino acid sequence having 2 amino acid moieties;
D is selected from

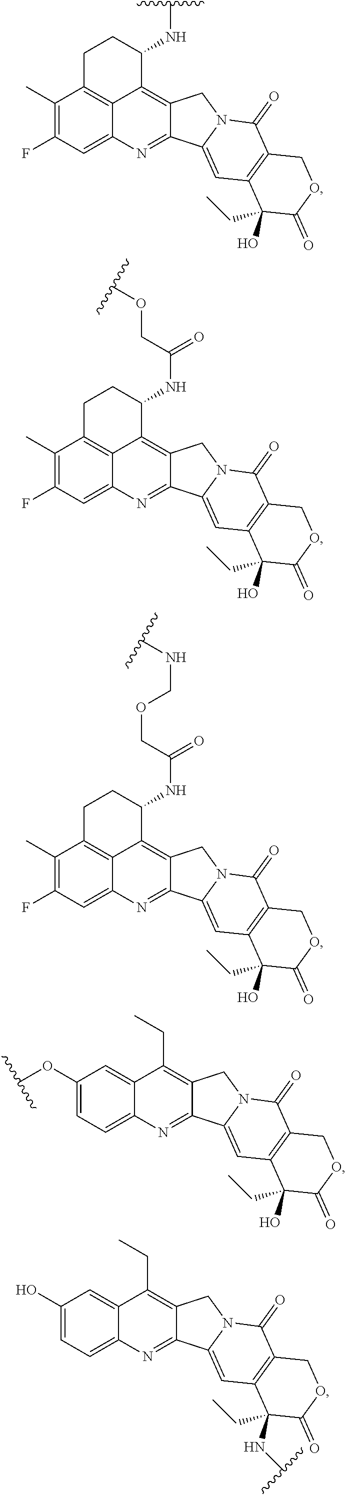 [SMILES: *N[C@H]1CCc2c(C)c(F)cc3nc4c(c1c23)Cn1c-4cc2c(c1=O)COC(=O)[C@]2(O)CC ; *OCC(=O)N[C@H]1CCc2c(C)c(F)cc3nc4c(c1c23)Cn1c-4cc2c(c1=O)COC(=O)[C@]2(O)CC ; *NCOCC(=O)N[C@H]1CCc2c(C)c(F)cc3nc4c(c1c23)Cn1c-4cc2c(c1=O)COC(=O)[C@]2(O)CC ; *Oc1ccc2nc3c(c(CC)c2c1)Cn1c-3cc2c(c1=O)COC(=O)[C@]2(O)CC ; *N[C@]1(CC)C(=O)OCc2c1cc1n(c2=O)Cc2c-1nc1ccc(O)cc1c2CC]

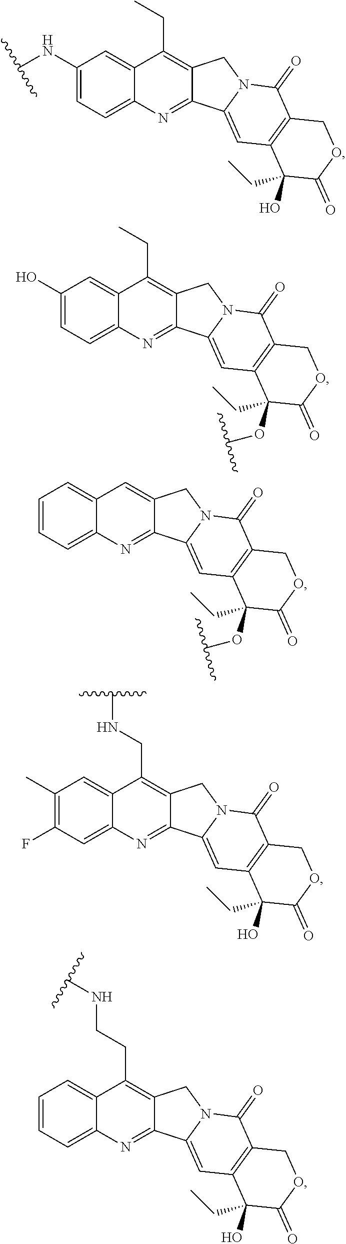 [SMILES: *Nc1ccc2nc3c(c(CC)c2c1)Cn1c-3cc2c(c1=O)COC(=O)[C@]2(O)CC ; *O[C@]1(CC)C(=O)OCc2c1cc1n(c2=O)Cc2c-1nc1ccc(O)cc1c2CC ; *O[C@]1(CC)C(=O)OCc2c1cc1n(c2=O)Cc2cc3ccccc3nc2-1 ; *NCc1c2c(nc3cc(F)c(C)cc13)-c1cc3c(c(=O)n1C2)COC(=O)[C@]3(O)CC ; *NCCc1c2c(nc3ccccc13)-c1cc3c(c(=O)n1C2)COC(=O)[C@]3(O)CC]

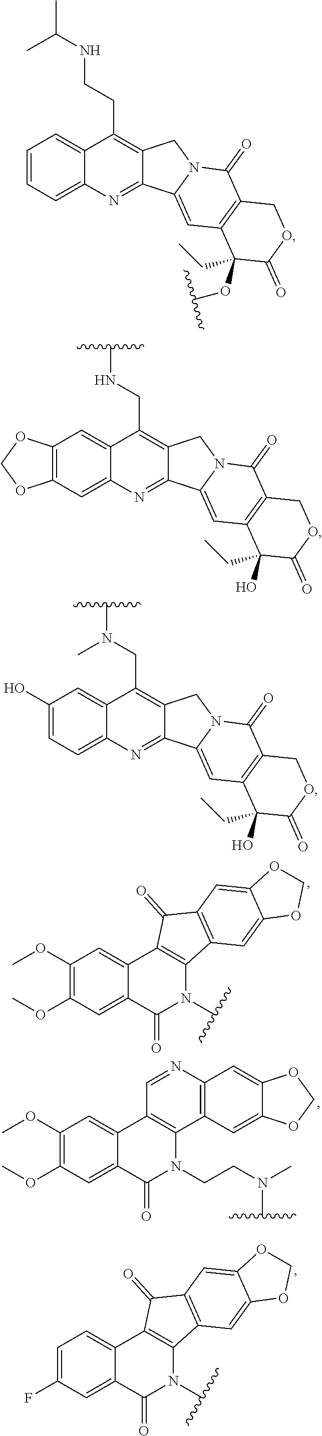

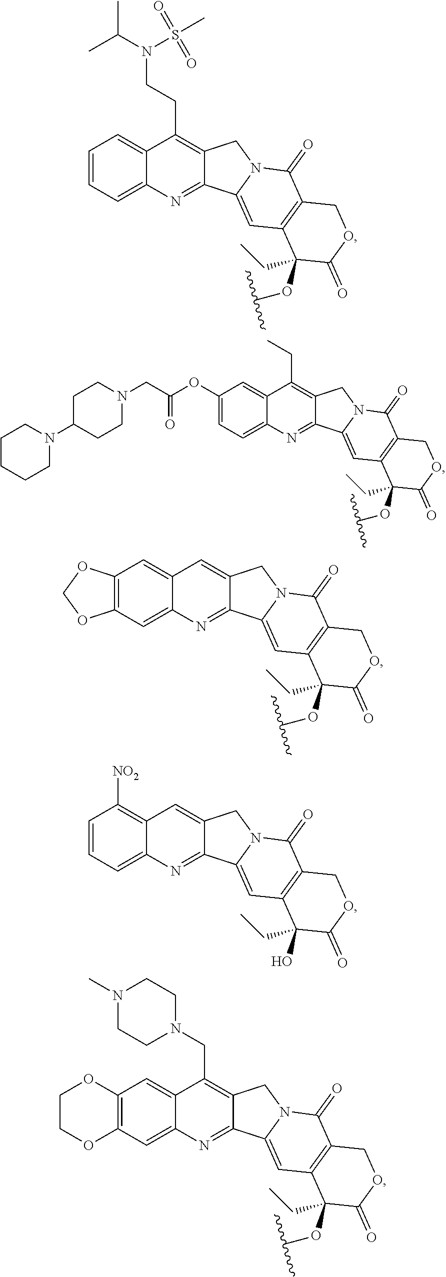

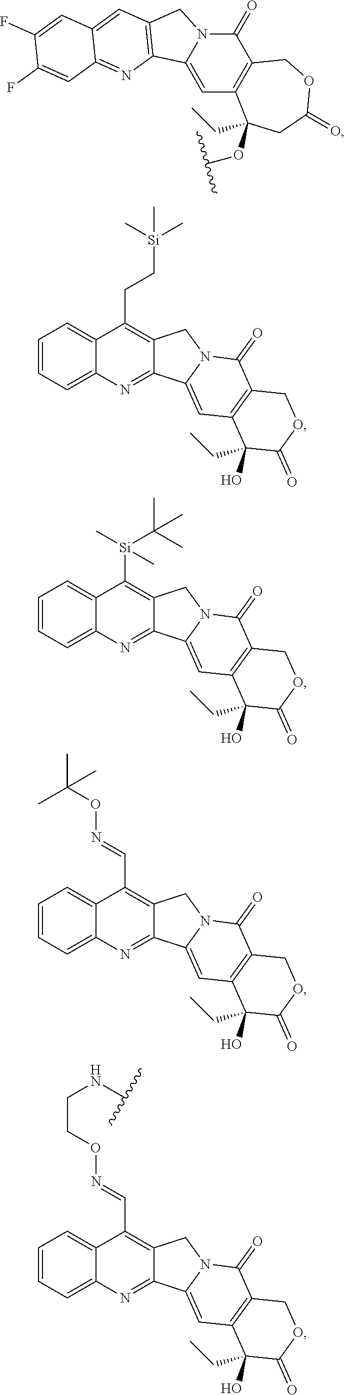 [SMILES: *O[C@]1(CC)CC(=O)OCc2c1cc1n(c2=O)Cc2cc3cc(F)c(F)cc3nc2-1 ; CC[C@@]1(O)C(=O)OCc2c1cc1n(c2=O)Cc2c-1nc1ccccc1c2CC[Si](C)(C)C ; CC[C@@]1(O)C(=O)OCc2c1cc1n(c2=O)Cc2c-1nc1ccccc1c2[Si](C)(C)C(C)(C)C ; CC[C@@]1(O)C(=O)OCc2c1cc1n(c2=O)Cc2c-1nc1ccccc1c2/C=N/OC(C)(C)C ; *NCCO/N=C/c1c2c(nc3ccccc13)-c1cc3c(c(=O)n1C2)COC(=O)[C@]3(O)CC]

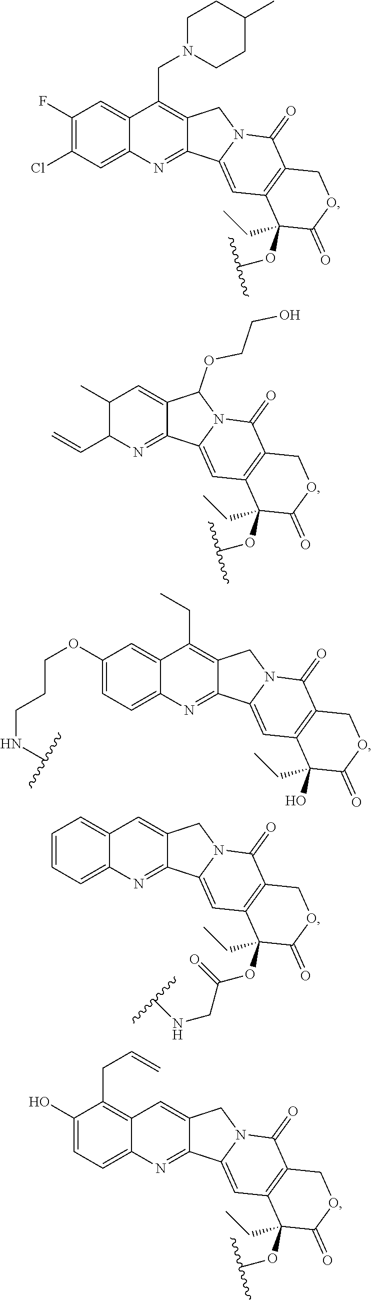

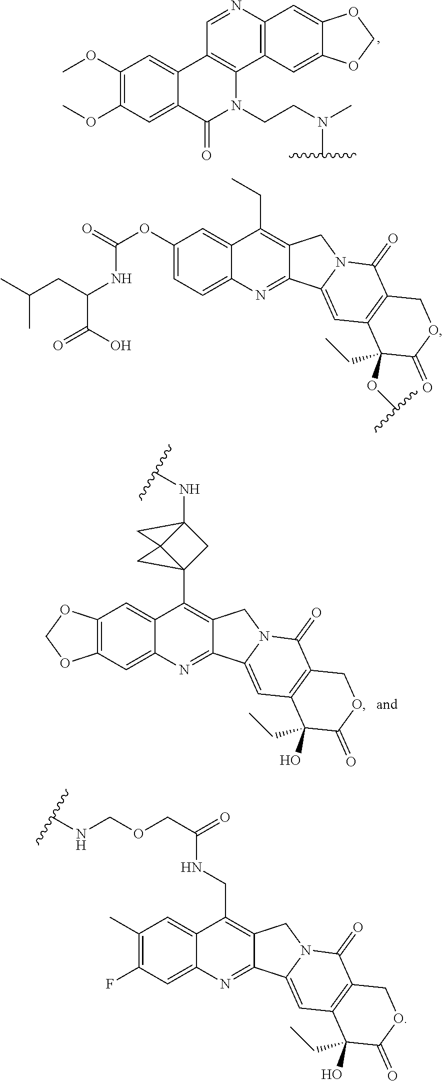

2. The antibody-drug conjugate of claim 1, wherein L₁ is

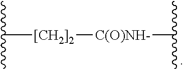

3. The antibody-drug conjugate of claim 1 or 2, wherein p is 7 or 8.

4. The antibody-drug conjugate of any one of claims 1-3, wherein p is 8.

5. The antibody-drug conjugate of any one of claims 1-4, wherein X_{AA} is selected from Val-Ala, Tyr-Arg, Phe-Arg, Val-Gln, Val-Cit, Tyr-Met, Leu-Gln, Val-Arg, Met-Thr, Phe-Gln, Thr-Thr, Val-Thr, Ala-Ala, Val-Met, Leu-Met, Ala-Asn, D-Val-D-Gln, D-Ala-D-Ala, and Phe-Met.

6. The antibody-drug conjugate of any one of claims 1-5, wherein X_{AA} is valine-alanine.

7. The antibody-drug conjugate of any one of claims 1-6, wherein L_{A} is —[CH₂CH₂O]ₚ—(CH₂)₁₋₃—C(O)—X_{AA}—.

8. The antibody-drug conjugate of any one of claims 1-7, wherein L_{A} is —[CH₂CH₂O]ₚ—(CH₂)₂—C(O)—X_{AA}—.

9. The antibody-drug conjugate of any one of claims 1-8, wherein the linker L has the formula:

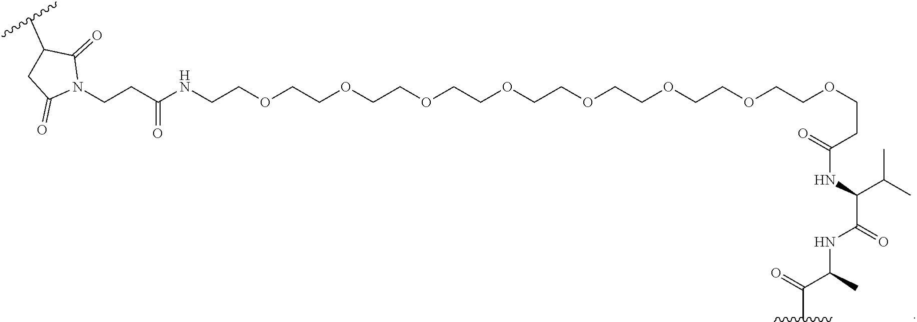 [SMILES: *C(=O)[C@H](C)NC(=O)[C@@H](NC(=O)CCOCCOCCOCCOCCOCCOCCOCCOCCNC(=O)CCN1C(=O)CC(*)C1=O)C(C)C]

10. The antibody-drug conjugate of any one of claims 1-9, wherein D is

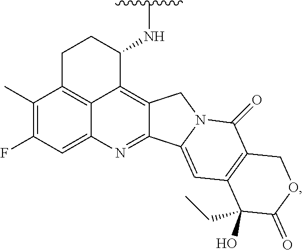 [SMILES: *N[C@H]1CCc2c(C)c(F)cc3nc4c(c1c23)Cn1c-4cc2c(c1=O)COC(=O)[C@]2(O)CC]

11. The antibody-drug conjugate of any one of claims 1-10, wherein L-D has the formula:

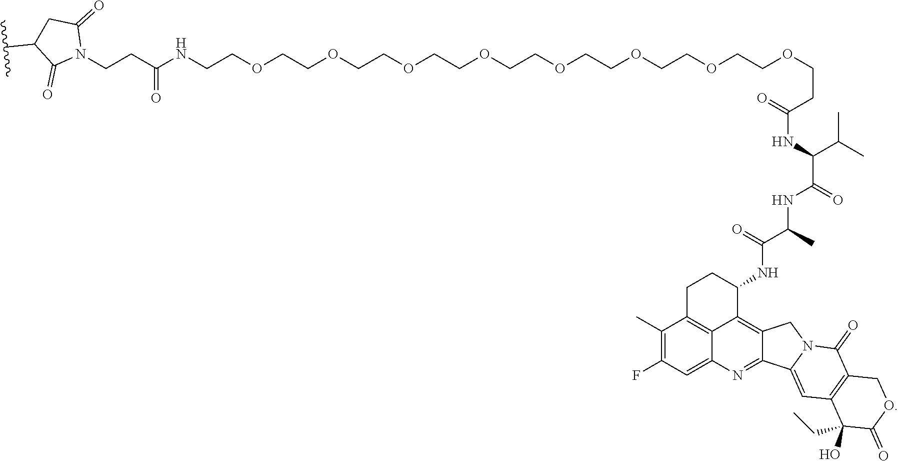 [SMILES: *C1CC(=O)N(CCC(=O)NCCOCCOCCOCCOCCOCCOCCOCCOCCC(=O)N[C@H](C(=O)N[C@@H](C)C(=O)N[C@H]2CCc3c(C)c(F)cc4nc5c(c2c34)Cn2c-5cc3c(c2=O)COC(=O)[C@]3(O)CC)C(C)C)C1=O]

12. The antibody-drug conjugate of any one of claims 1-11, wherein n is an integer from 4 to 8.

13. The antibody-drug conjugate of claim 12, wherein n is 4.

14. The antibody-drug conjugate of claim 12, wherein n is 8.

15. The antibody-drug conjugate of any one of claims 1-14, wherein the antibody or binding fragment thereof comprises:
(i) a heavy chain variable region (VH) that comprises:
(a) a VH complementarity determining region one (CDRH1) comprising and/or consisting of the amino acid sequence of SEQ ID NO: 2,
(b) a VH complementarity determining region two (CDRH2) comprising and/or consisting of the amino acid sequence of SEQ ID NO: 3, and
(c) a VH complementarity determining region three (CDRH3) comprising and/or consisting of the amino acid sequence of SEQ ID NO: 4,
and (ii) a light chain variable region (VL) that comprises:
(a) a VL complementarity determining region one (CDRL1) comprising and/or consisting of the amino acid sequence of SEQ ID NO: 6,
(b) a VL complementarity determining region two (CDRL2) comprising and/or consisting of the amino acid sequence of SEQ ID NO: 7, and
(c) a VL complementarity determining region three (CDRL3) comprising and/or consisting of the amino acid sequence of SEQ ID NO:8.

16. The antibody-drug conjugate of any one of claims 1-14, wherein the antibody or binding fragment thereof comprises:
(i) a heavy chain variable region (VH) that comprises:
(a) a VH complementarity determining region one (CDRH1) comprising and/or consisting of the amino acid sequence of SEQ ID NO: 12,
(b) a VH complementarity determining region two (CDRH2) comprising and/or consisting of the amino acid sequence of SEQ ID NO: 13, and
(c) a VH complementarity determining region three (CDRH3) comprising and/or consisting of the amino acid sequence of SEQ ID NO: 14,
and (ii) a light chain variable region (VL) that comprises:
(a) a VL complementarity determining region one (CDRL1) comprising and/or consisting of the amino acid sequence of SEQ ID NO: 6,
(b) a VL complementarity determining region two (CDRL2) comprising and/or consisting of the amino acid sequence of SEQ ID NO: 7, and
(c) a VL complementarity determining region three (CDRL3) comprising and/or consisting of the amino acid sequence of SEQ ID NO:8.

17. The antibody-drug conjugate of any one of claims 1-16, wherein the antibody or binding fragment thereof comprises a VH that comprises an amino acid sequence at least 90%, at least 91%, at least 92%, at least 93%, at least 94%, at least 95%, at least 96%, at least 97%, at least 98%, at least 99%, or 100% identical to the amino acid sequence of SEQ ID NO: 1, and/or a VL that comprises an amino acid sequence at least 90%, at least 91%, at least 92%, at least 93%, at least 94%, at least 95%, at least 96%, at least 97%, at least 98%, at least 99%, or 100% identical to the amino acid sequence of SEQ ID NO: 5.

18. The antibody-drug conjugate of claim 17, wherein the antibody or binding fragment thereof comprises a VH that comprises the amino acid sequence of SEQ ID NO: 1 and/or a VL that comprises the amino acid sequence of SEQ ID NO: 5.

19. The antibody-drug conjugate of any one of claims 1-18, wherein the antibody or binding fragment thereof comprises a heavy chain comprising an amino acid sequence at least 90%, at least 91%, at least 92%, at least 93%, at least 94%, at least 95%, at least 96%, at least 97%, at least 98%, at least 99%, or 100% identical to the amino acid sequence of SEQ ID NO: 1 and/or a light chain comprising an amino acid sequence at least 90%, at least 91%, at least 92%, at least 93%, at least 94%, at least 95%, at least 96%, at least 97%, at least 98%, at least 99%, or 100% identical to the amino acid sequence of SEQ ID NO: 5.

20. The antibody-drug conjugate of claim 19, wherein the antibody or binding fragment thereof comprises a heavy chain that comprises the amino acid sequence of SEQ ID NO: 1 and/or a light chain that comprises the amino acid sequence of SEQ ID NO: 5.

21. The antibody-drug conjugate of any one of claims 1-20, wherein the antibody-drug conjugate has a drug-to-antibody ratio (DAR) ranging from about 1 to about 10, optionally wherein the DAR is about 1, about 2, about 3, about 4, about 5, about 6, about 7, about 8, about 9, or about 10, optionally DAR is about 4, optionally DAR is about 8.

22. A pharmaceutical composition comprising the antibody drug conjugate of any one of claims 1-21; and a pharmaceutically acceptable carrier.

23. A method of treating a cancer comprising administering to a subject in need thereof a therapeutically effective amount of the antibody drug conjugate of any one of claims 1-21, or the pharmaceutical composition of claim 22.

24. The method of claim 23, wherein less than about 50% of the antibody-drug conjugate is converted to a metabolite about 24 hours after administering the therapeutically effective amount of the antibody-drug conjugate to the subject.

25. The method of claim 23 or 24, wherein about 50% of the antibody-drug conjugate is converted to a metabolite about 96 hours after administering the therapeutically effective amount of the antibody-drug conjugate to the subject.

26. The method of any one of claims 23-25, wherein the antibody-drug conjugate is converted to a metabolite of formula 300:

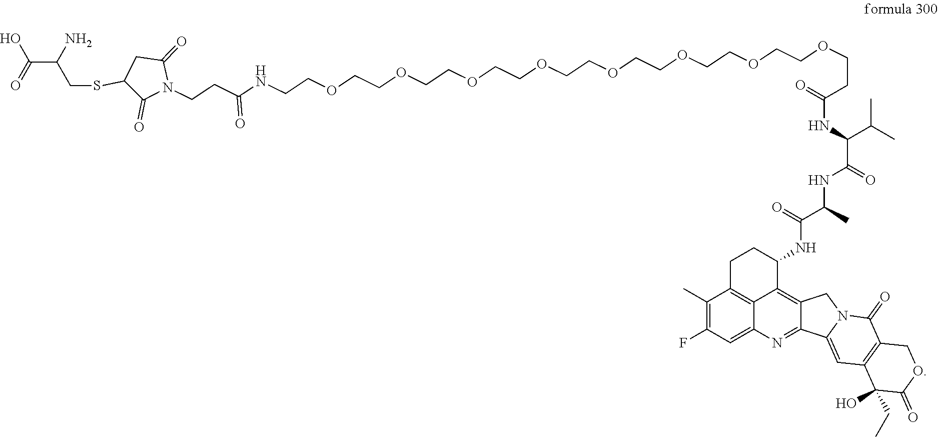 [SMILES: CC[C@@]1(O)C(=O)OCc2c1cc1n(c2=O)Cc2c-1nc1cc(F)c(C)c3c1c2[C@@H](NC(=O)[C@H](C)NC(=O)[C@@H](NC(=O)CCOCCOCCOCCOCCOCCOCCOCCOCCNC(=O)CCN1C(=O)CC(SCC(N)C(=O)O)C1=O)C(C)C)CC3]

27. The method of any one of claims 23-26, wherein the antibody-drug conjugate is converted to a metabolite of formula 301:

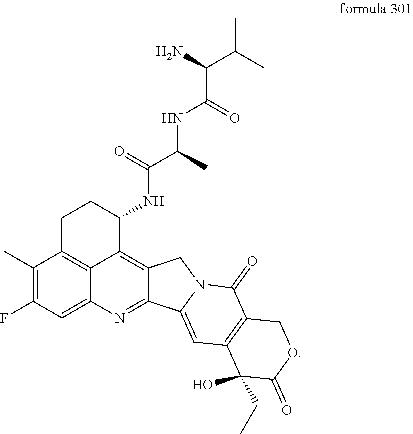 [SMILES: CC[C@@]1(O)C(=O)OCc2c1cc1n(c2=O)Cc2c-1nc1cc(F)c(C)c3c1c2[C@@H](NC(=O)[C@H](C)NC(=O)[C@@H](N)C(C)C)CC3]

28. The method of any one of claims 23-27, wherein the antibody-drug conjugate is converted to a metabolite of formula 302:

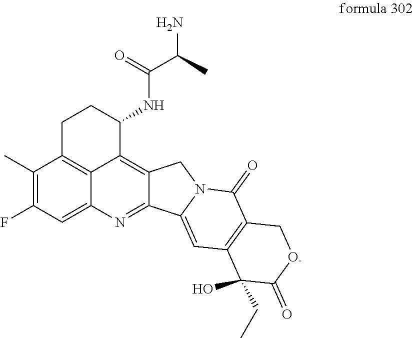 [SMILES: CC[C@@]1(O)C(=O)OCc2c1cc1n(c2=O)Cc2c-1nc1cc(F)c(C)c3c1c2[C@@H](NC(=O)[C@H](C)N)CC3]

29. The method of any one of claims 23-28, wherein the antibody-drug conjugate is converted to a metabolite in vivo.

30. The method of any one of claims 23-28, wherein the antibody-drug conjugate is converted to a metabolite in vitro.

31. The method of any one of claims 23-30, wherein the cancer is selected from the group consisting of pancreatic cancer, breast cancer, prostate cancer, lymphoma, skin cancer, colon cancer, melanoma, malignant melanoma, ovarian cancer, brain cancer, primary brain carcinoma, head-neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, chronic lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, or retinoblastoma, and the like. In other embodiments, the cancer is acoustic neuroma, adenocarcinoma, angiosarcoma, astrocytoma, basal cell carcinoma, bile duct carcinoma, bladder carcinoma, brain cancer, breast cancer, triple-negative breast cancer (TNBC), bronchogenic carcinoma, cervical cancer, chordoma, choriocarcinoma, colon cancer, colorectal cancer, craniopharyngioma, cystadenocarcinoma, embryonal carcinoma, endotheliocarcinoma, ependymoma, epithelial carcinoma, esophageal cancer, Ewing's tumor, fibrosarcoma, gastric cancer, glioblastoma multiforme, glioma, head and neck cancer, hemangioblastoma, hepatoma, kidney cancer, leiomyosarcoma, liposarcoma, lung cancer, lymphangioendotheliosarcoma, lymphangiosarcoma, medullary carcinoma, medulloblastoma, melanoma, meningioma, mesothelioma, myxosarcoma, nasal cancer, neuroblastoma, oligodendroglioma, oral cancer, osteogenic sarcoma, ovarian cancer, pancreatic cancer, papillary adenocarcinoma, papillary carcinoma, pinealoma, prostate cancer, rabdomyosarcoma, rectal cancer, renal cell carcinoma, retinoblastoma, sarcoma, sebaceous gland carcinoma, seminoma, skin cancer, squamous cell carcinoma, stomach cancer, sweat gland carcinoma, synovioma, testicular cancer, small cell lung carcinoma, throat cancer, uterine cancer, Wilm's tumor, blood cancer, acute erythroleukemic leukemia, acute lymphoblastic B-cell leukemia, acute lymphoblastic T-cell leukemia, acute lymphoblastic leukemia, acute megakaryoblastic leukemia, acute monoblastic leukemia, acute myeloblastic leukemia, acute myelomonocytic leukemia, acute nonlymphocytic leukemia, acute promyelocytic leukemia, acute undifferentiated leukemia, chronic lymphocytic leukemia, chronic myelocytic leukemia, hairy cell leukemia, multiple myeloma, heavy chain disease, Hodgkin's disease, multiple myeloma, non-Hodgkin's lymphoma, polycythemia vera, or Waldenstrom's macroglobulinemia.

32. The method of any one of claims 23-31, wherein the cancer in triple-negative breast cancer (TNBC).

33. An antibody-drug conjugate having formula (I): 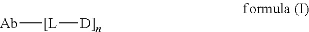
wherein in formula (I):
Ab comprises an antibody or antibody-binding fragment, wherein the antibody or binding fragment thereof specifically binds CUB Domain-Containing Protein-1 (CDCP1);
D comprises a drug moiety;
n is an integer from 1 to 20; and
L has the formula:

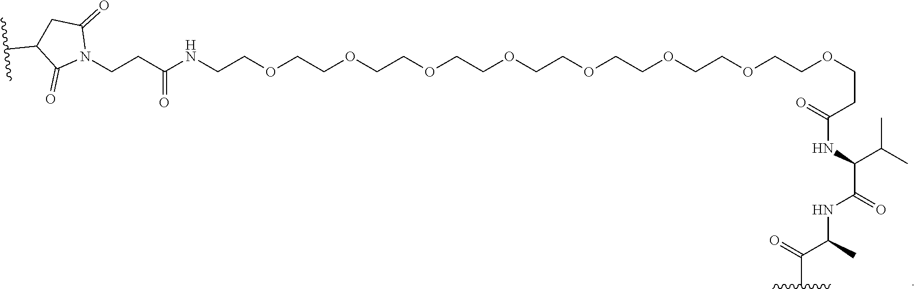 [SMILES: *C(=O)[C@H](C)NC(=O)[C@@H](NC(=O)CCOCCOCCOCCOCCOCCOCCOCCOCCNC(=O)CCN1C(=O)CC(*)C1=O)C(C)C]

34. The antibody-drug conjugate of claim 33, wherein the antibody or binding fragment thereof comprises:
(i) a heavy chain variable region (VH) that comprises:
(a) a VH complementarity determining region one (CDRH1) comprising and/or consisting of the amino acid sequence of SEQ ID NO: 2,
(b) a VH complementarity determining region two (CDRH2) comprising and/or consisting of the amino acid sequence of SEQ ID NO: 3, and
(c) a VH complementarity determining region three (CDRH3) comprising and/or consisting of the amino acid sequence of SEQ ID NO: 4,
and (ii) a light chain variable region (VL) that comprises:
(a) a VL complementarity determining region one (CDRL1) comprising and/or consisting of the amino acid sequence of SEQ ID NO: 6,
(b) a VL complementarity determining region two (CDRL2) comprising and/or consisting of the amino acid sequence of SEQ ID NO: 7, and
(c) a VL complementarity determining region three (CDRL3) comprising and/or consisting of the amino acid sequence of SEQ ID NO:8.

35. The antibody-drug conjugate of claim 33, wherein the antibody or binding fragment thereof comprises:
(i) a heavy chain variable region (VH) that comprises:
(a) a VH complementarity determining region one (CDRH1) comprising and/or consisting of the amino acid sequence of SEQ ID NO: 12,
(b) a VH complementarity determining region two (CDRH2) comprising and/or consisting of the amino acid sequence of SEQ ID NO: 13, and
(c) a VH complementarity determining region three (CDRH3) comprising and/or consisting of the amino acid sequence of SEQ ID NO: 14,
and (ii) a light chain variable region (VL) that comprises:
(a) a VL complementarity determining region one (CDRL1) comprising and/or consisting of the amino acid sequence of SEQ ID NO: 6,
(b) a VL complementarity determining region two (CDRL2) comprising and/or consisting of the amino acid sequence of SEQ ID NO: 7, and
(c) a VL complementarity determining region three (CDRL3) comprising and/or consisting of the amino acid sequence of SEQ ID NO:8.

36. The antibody-drug conjugate of any one of claims 34-36, wherein the drug moiety is selected from

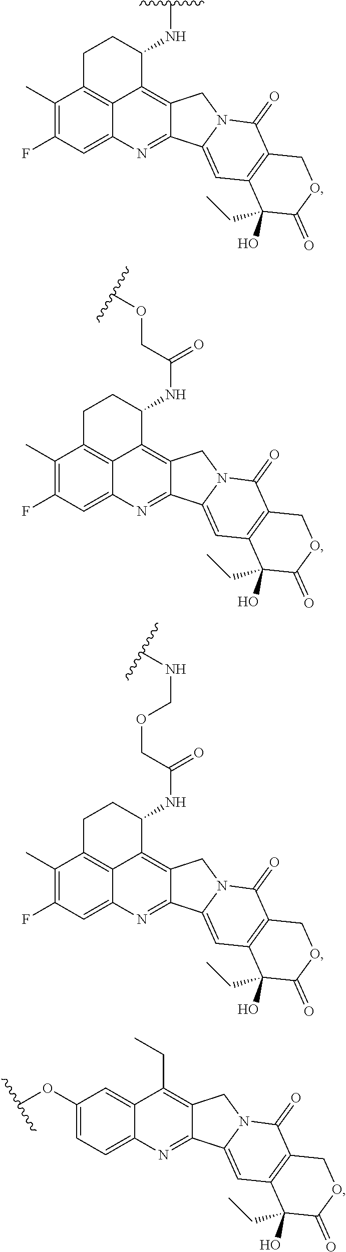 [SMILES: *N[C@H]1CCc2c(C)c(F)cc3nc4c(c1c23)Cn1c-4cc2c(c1=O)COC(=O)[C@]2(O)CC ; *OCC(=O)N[C@H]1CCc2c(C)c(F)cc3nc4c(c1c23)Cn1c-4cc2c(c1=O)COC(=O)[C@]2(O)CC ; *NCOCC(=O)N[C@H]1CCc2c(C)c(F)cc3nc4c(c1c23)Cn1c-4cc2c(c1=O)COC(=O)[C@]2(O)CC ; *Oc1ccc2nc3c(c(CC)c2c1)Cn1c-3cc2c(c1=O)COC(=O)[C@]2(O)CC]

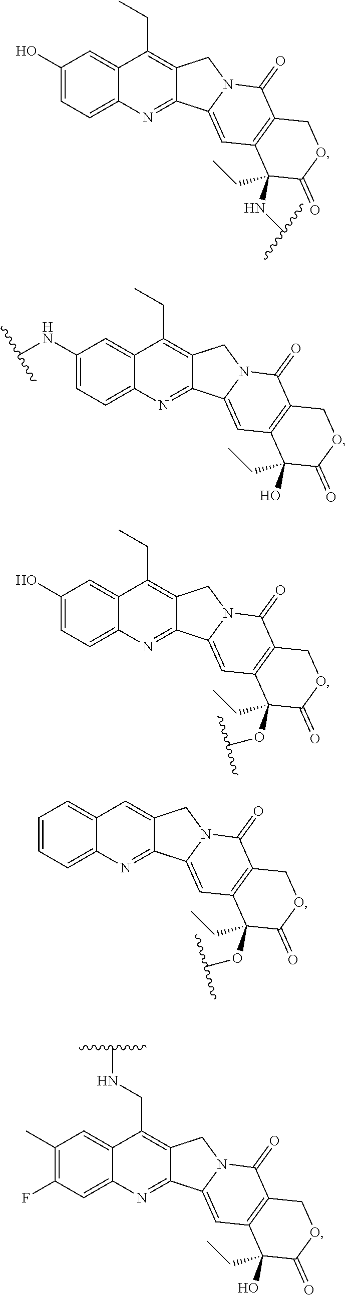 [SMILES: *N[C@]1(CC)C(=O)OCc2c1cc1n(c2=O)Cc2c-1nc1ccc(O)cc1c2CC ; *Nc1ccc2nc3c(c(CC)c2c1)Cn1c-3cc2c(c1=O)COC(=O)[C@]2(O)CC ; *O[C@]1(CC)C(=O)OCc2c1cc1n(c2=O)Cc2c-1nc1ccc(O)cc1c2CC ; *O[C@]1(CC)C(=O)OCc2c1cc1n(c2=O)Cc2cc3ccccc3nc2-1 ; *NCc1c2c(nc3cc(F)c(C)cc13)-c1cc3c(c(=O)n1C2)COC(=O)[C@]3(O)CC]

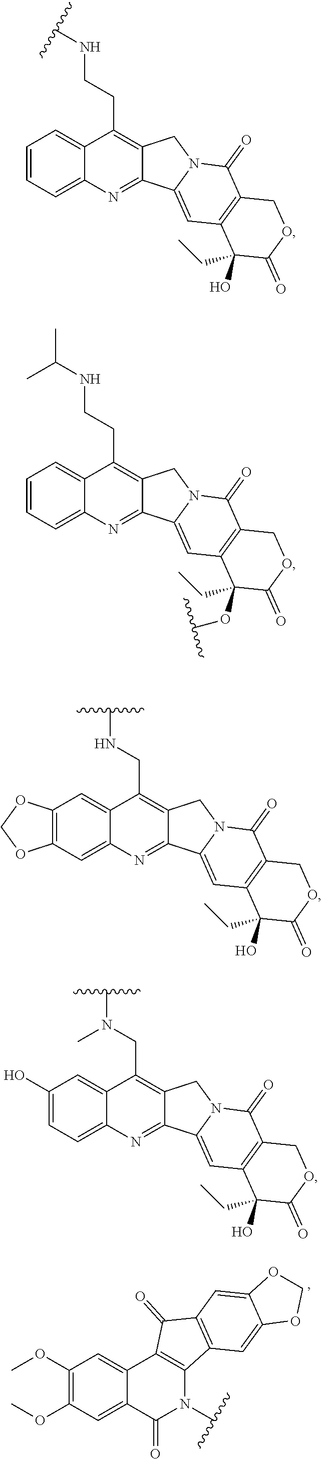 [SMILES: *NCCc1c2c(nc3ccccc13)-c1cc3c(c(=O)n1C2)COC(=O)[C@]3(O)CC ; *O[C@]1(CC)C(=O)OCc2c1cc1n(c2=O)Cc2c-1nc1ccccc1c2CCNC(C)C ; *NCc1c2c(nc3cc4c(cc13)OCO4)-c1cc3c(c(=O)n1C2)COC(=O)[C@]3(O)CC ; *N(C)Cc1c2c(nc3ccc(O)cc13)-c1cc3c(c(=O)n1C2)COC(=O)[C@]3(O)CC ; *n1c2c(c3cc(OC)c(OC)cc3c1=O)C(=O)c1cc3c(cc1-2)OCO3]

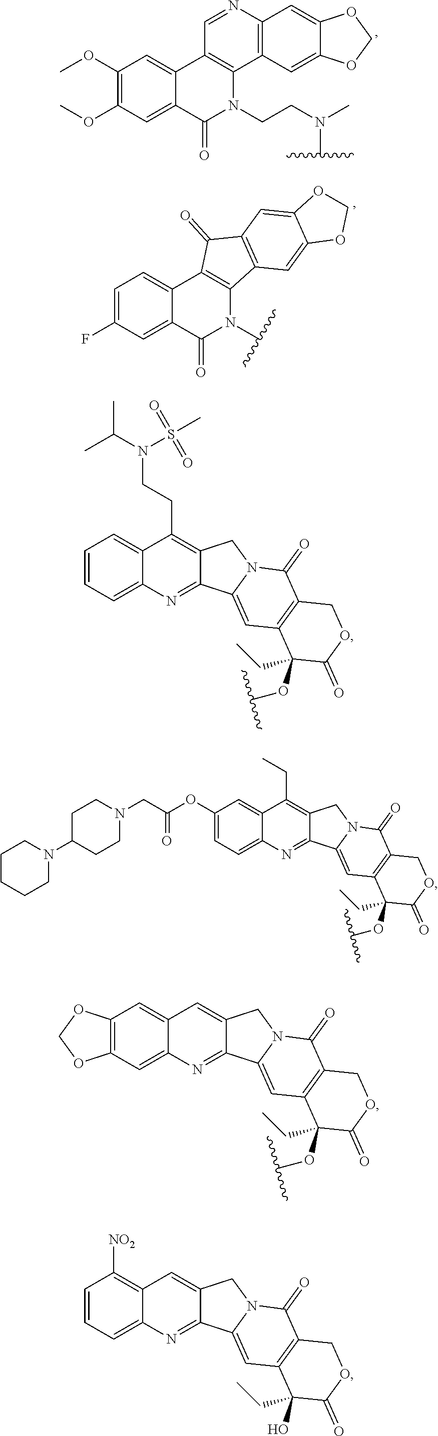

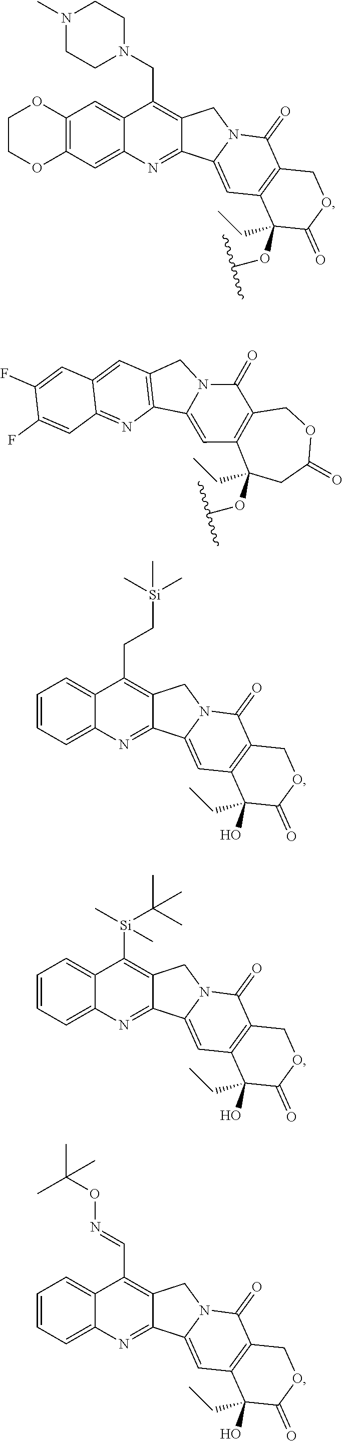 [SMILES: *O[C@]1(CC)C(=O)OCc2c1cc1n(c2=O)Cc2c-1nc1cc3c(cc1c2CN1CCN(C)CC1)OCCO3 ; *O[C@]1(CC)CC(=O)OCc2c1cc1n(c2=O)Cc2cc3cc(F)c(F)cc3nc2-1 ; CC[C@@]1(O)C(=O)OCc2c1cc1n(c2=O)Cc2c-1nc1ccccc1c2CC[Si](C)(C)C ; CC[C@@]1(O)C(=O)OCc2c1cc1n(c2=O)Cc2c-1nc1ccccc1c2[Si](C)(C)C(C)(C)C ; CC[C@@]1(O)C(=O)OCc2c1cc1n(c2=O)Cc2c-1nc1ccccc1c2/C=N/OC(C)(C)C]

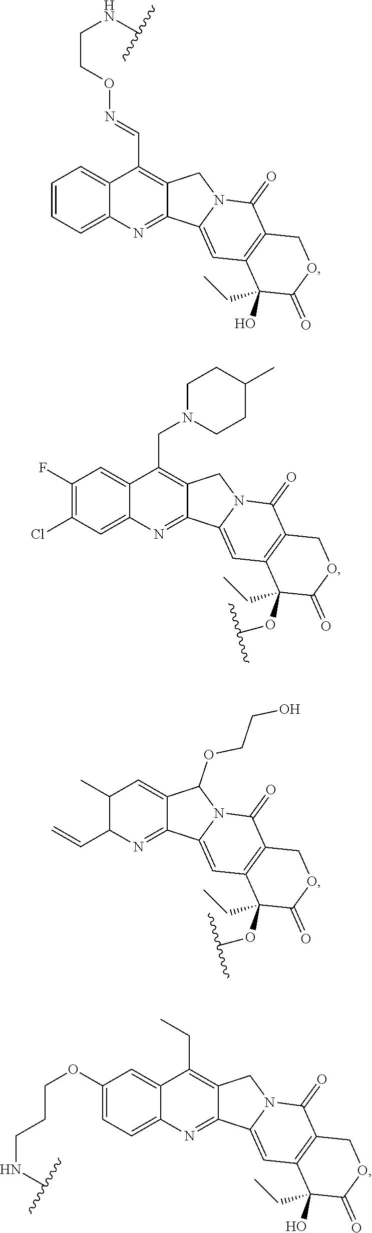 [SMILES: *NCCO/N=C/c1c2c(nc3ccccc13)-c1cc3c(c(=O)n1C2)COC(=O)[C@]3(O)CC ; *O[C@]1(CC)C(=O)OCc2c1cc1n(c2=O)Cc2c-1nc1cc(Cl)c(F)cc1c2CN1CCC(C)CC1 ; *O[C@]1(CC)C(=O)OCc2c1cc1n(c2=O)C(OCCO)C2=CC(C)C(C=C)N=C21 ; *NCCCOc1ccc2nc3c(c(CC)c2c1)Cn1c-3cc2c(c1=O)COC(=O)[C@]2(O)CC]

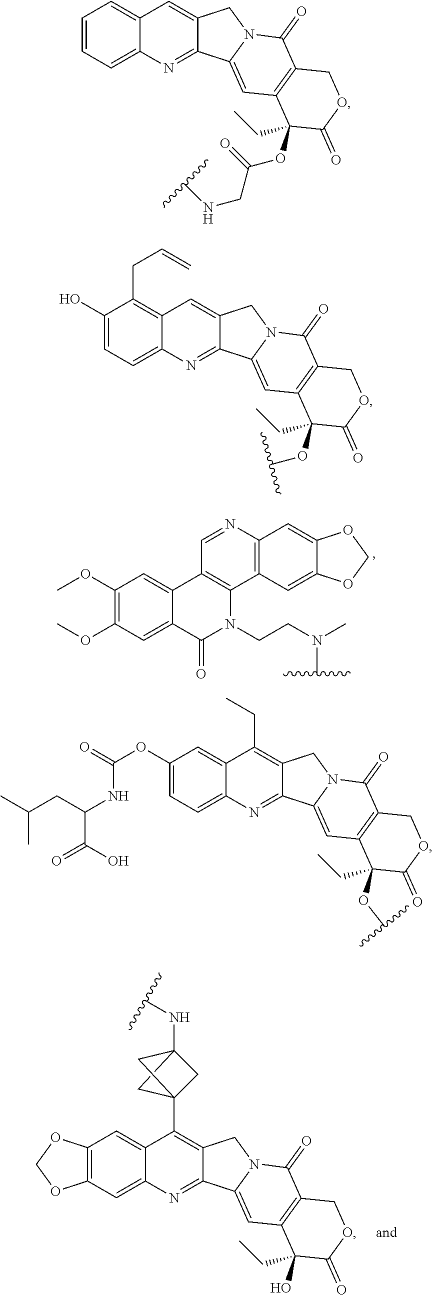

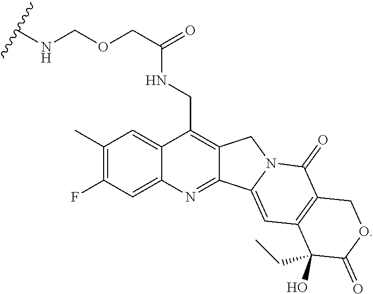 [SMILES: *NCOCC(=O)NCc1c2c(nc3cc(F)c(C)cc13)-c1cc3c(c(=O)n1C2)COC(=O)[C@]3(O)CC]

37. The antibody-drug conjugate of any one of claims 33-36, wherein the drug moiety is selected from

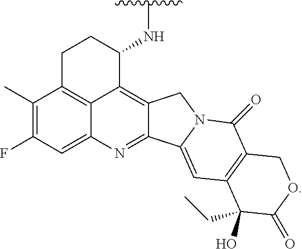 [SMILES: *N[C@H]1CCc2c(C)c(F)cc3nc4c(c1c23)Cn1c-4cc2c(c1=O)COC(=O)[C@]2(O)CC]

38. The antibody-drug conjugate of any one of claims 33-37, wherein the antibody-drug conjugate has a drug-to-antibody ratio (DAR) ranging from about 1 to about 10, optionally wherein the DAR is about 1, about 2, about 3, about 4, about 5, about 6, about 7, about 8, about 9, or about 10, optionally DAR is about 4, optionally DAR is about 8.

39. An antibody-drug conjugate having formula (I): 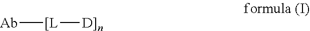
wherein in formula (I):
Ab comprises an antibody or antibody-binding fragment, wherein the antibody or binding fragment thereof specifically binds CUB Domain-Containing Protein-1 (CDCP1);
n is an integer from 1 to 20; and
L-D has the formula:

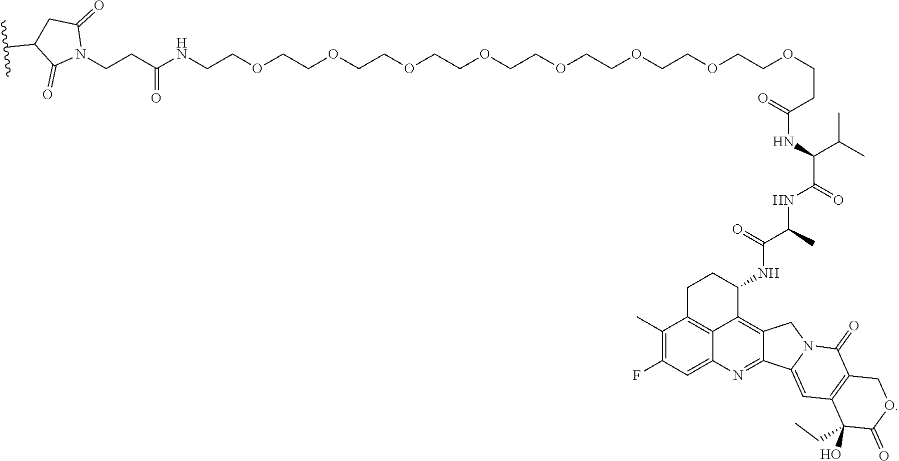 [SMILES: *C1CC(=O)N(CCC(=O)NCCOCCOCCOCCOCCOCCOCCOCCOCCC(=O)N[C@H](C(=O)N[C@@H](C)C(=O)N[C@H]2CCc3c(C)c(F)cc4nc5c(c2c34)Cn2c-5cc3c(c2=O)COC(=O)[C@]3(O)CC)C(C)C)C1=O]

40. The antibody-drug conjugate of claim 39, wherein the antibody or binding fragment thereof comprises:
(i) a heavy chain variable region (VH) that comprises:
(a) a VH complementarity determining region one (CDRH1) comprising and/or consisting of the amino acid sequence of SEQ ID NO: 2,
(b) a VH complementarity determining region two (CDRH2) comprising and/or consisting of the amino acid sequence of SEQ ID NO: 3, and
(c) a VH complementarity determining region three (CDRH3) comprising and/or consisting of the amino acid sequence of SEQ ID NO: 4,
and (ii) a light chain variable region (VL) that comprises:
(a) a VL complementarity determining region one (CDRL1) comprising and/or consisting of the amino acid sequence of SEQ ID NO: 6,
(b) a VL complementarity determining region two (CDRL2) comprising and/or consisting of the amino acid sequence of SEQ ID NO: 7, and
(c) a VL complementarity determining region three (CDRL3) comprising and/or consisting of the amino acid sequence of SEQ ID NO:8.

41. The antibody-drug conjugate of claim 40, wherein the antibody or binding fragment thereof comprises:
(i) a heavy chain variable region (VH) that comprises:
(a) a VH complementarity determining region one (CDRH1) comprising and/or consisting of the amino acid sequence of SEQ ID NO: 12,
(b) a VH complementarity determining region two (CDRH2) comprising and/or consisting of the amino acid sequence of SEQ ID NO: 13, and
(c) a VH complementarity determining region three (CDRH3) comprising and/or consisting of the amino acid sequence of SEQ ID NO: 14,
and (ii) a light chain variable region (VL) that comprises:
(a) a VL complementarity determining region one (CDRL1) comprising and/or consisting of the amino acid sequence of SEQ ID NO: 6,
(b) a VL complementarity determining region two (CDRL2) comprising and/or consisting of the amino acid sequence of SEQ ID NO: 7, and
(c) a VL complementarity determining region three (CDRL3) comprising and/or consisting of the amino acid sequence of SEQ ID NO:8.

42. The antibody-drug conjugate of any one of claims 39-42, wherein the antibody-drug conjugate has a drug-to-antibody ratio (DAR) ranging from about 1 to about 10, optionally wherein the DAR is about 1, about 2, about 3, about 4, about 5, about 6, about 7, about 8, about 9, or about 10, optionally DAR is about 4, optionally DAR is about 8.

43. The antibody-drug conjugate of any one of claims 39-42, wherein n is an integer from 1 to 10, 2 to 8, or 4 to 8, optionally n is 1, 2, 3, 4, 5, 6, 7, 8, 9, or 10, optionally n is 4 or 8, optionally n is 4, optionally n is 8.

44. An antibody-drug conjugate having any one of formula 1030-1064 or 1100-1118, wherein Ab comprises an antibody or antibody-binding fragment, wherein the antibody or binding fragment thereof specifically binds CUB Domain-Containing Protein-1 (CDCP1).

45. An antibody-drug conjugate of any one of embodiments (I)-(XVII).

46. An antibody-drug conjugate having formula (I): 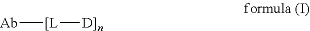
wherein in formula (I):
Ab comprises an antibody or antibody-binding fragment, wherein the antibody or binding fragment thereof specifically binds CUB Domain-Containing Protein-1 (CDCP1);
n is 1;
L-D has the formula: 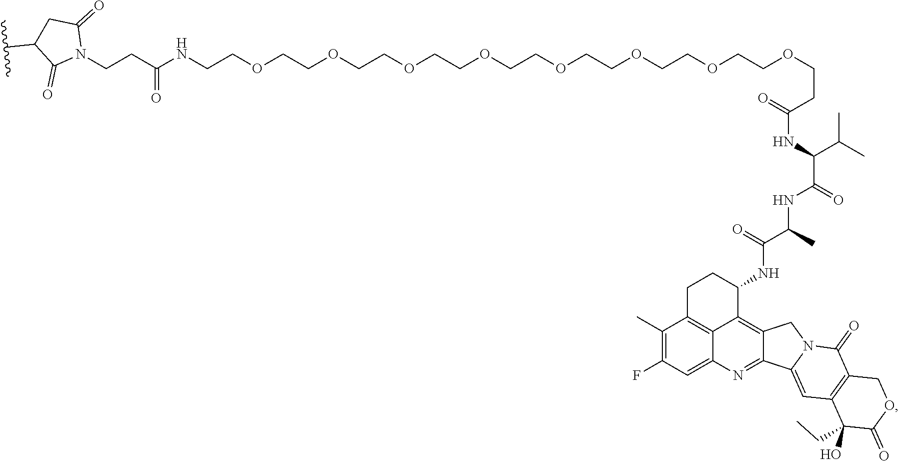 [SMILES: *C1CC(=O)N(CCC(=O)NCCOCCOCCOCCOCCOCCOCCOCCOCCC(=O)N[C@H](C(=O)N[C@@H](C)C(=O)N[C@H]2CCc3c(C)c(F)cc4nc5c(c2c34)Cn2c-5cc3c(c2=O)COC(=O)[C@]3(O)CC)C(C)C)C1=O]
wherein the antibody or binding fragment thereof comprises:
(i) a heavy chain variable region (VH) that comprises:
(a) a VH complementarity determining region one (CDRH1) comprising and/or consisting of the amino acid sequence of SEQ ID NO: 2,
(b) a VH complementarity determining region two (CDRH2) comprising and/or consisting of the amino acid sequence of SEQ ID NO: 3, and
(c) a VH complementarity determining region three (CDRH3) comprising and/or consisting of the amino acid sequence of SEQ ID NO: 4,
and (ii) a light chain variable region (VL) that comprises:
(a) a VL complementarity determining region one (CDRL1) comprising and/or consisting of the amino acid sequence of SEQ ID NO: 6,
(b) a VL complementarity determining region two (CDRL2) comprising and/or consisting of the amino acid sequence of SEQ ID NO: 7, and
(c) a VL complementarity determining region three (CDRL3) comprising and/or consisting of the amino acid sequence of SEQ ID NO:8.

47. An antibody-drug conjugate having formula (I): 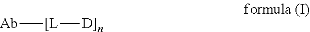
wherein in formula (I):
Ab comprises an antibody or antibody-binding fragment, wherein the antibody or binding fragment thereof specifically binds CUB Domain-Containing Protein-1 (CDCP1);
n is 4;
L-D has the formula: 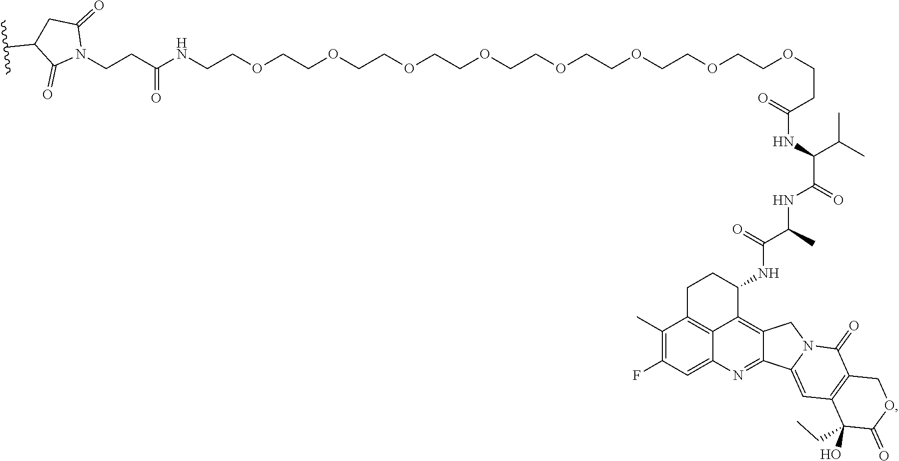 [SMILES: *C1CC(=O)N(CCC(=O)NCCOCCOCCOCCOCCOCCOCCOCCOCCC(=O)N[C@H](C(=O)N[C@@H](C)C(=O)N[C@H]2CCc3c(C)c(F)cc4nc5c(c2c34)Cn2c-5cc3c(c2=O)COC(=O)[C@]3(O)CC)C(C)C)C1=O]
wherein the antibody or binding fragment thereof comprises:
(i) a heavy chain variable region (VH) that comprises:
(a) a VH complementarity determining region one (CDRH1) comprising and/or consisting of the amino acid sequence of SEQ ID NO: 2,
(b) a VH complementarity determining region two (CDRH2) comprising and/or consisting of the amino acid sequence of SEQ ID NO: 3, and
(c) a VH complementarity determining region three (CDRH3) comprising and/or consisting of the amino acid sequence of SEQ ID NO: 4,
and (ii) a light chain variable region (VL) that comprises:
(a) a VL complementarity determining region one (CDRL1) comprising and/or consisting of the amino acid sequence of SEQ ID NO: 6,
(b) a VL complementarity determining region two (CDRL2) comprising and/or consisting of the amino acid sequence of SEQ ID NO: 7, and
(c) a VL complementarity determining region three (CDRL3) comprising and/or consisting of the amino acid sequence of SEQ ID NO:8.

48. An antibody-drug conjugate having formula (I): 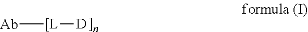
wherein in formula (I):
Ab comprises an antibody or antibody-binding fragment, wherein the antibody or binding fragment thereof specifically binds CUB Domain-Containing Protein-1 (CDCP1);
n is 8;
L-D has the formula: 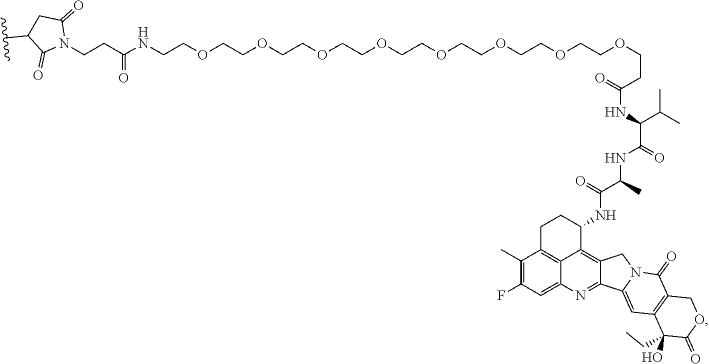 [SMILES: *C1CC(=O)N(CCC(=O)NCCOCCOCCOCCOCCOCCOCCOCCOCCC(=O)N[C@H](C(=O)N[C@@H](C)C(=O)N[C@H]2CCc3c(C)c(F)cc4nc5c(c2c34)Cn2c-5cc3c(c2=O)COC(=O)[C@]3(O)CC)C(C)C)C1=O]
wherein the antibody or binding fragment thereof comprises:
(i) a heavy chain variable region (VH) that comprises:
(a) a VH complementarity determining region one (CDRH1) comprising and/or consisting of the amino acid sequence of SEQ ID NO: 2,
(b) a VH complementarity determining region two (CDRH2) comprising and/or consisting of the amino acid sequence of SEQ ID NO: 3, and
(c) a VH complementarity determining region three (CDRH3) comprising and/or consisting of the amino acid sequence of SEQ ID NO: 4,
and (ii) a light chain variable region (VL) that comprises:
(a) a VL complementarity determining region one (CDRL1) comprising and/or consisting of the amino acid sequence of SEQ ID NO: 6,
(b) a VL complementarity determining region two (CDRL2) comprising and/or consisting of the amino acid sequence of SEQ ID NO: 7, and
(c) a VL complementarity determining region three (CDRL3) comprising and/or consisting of the amino acid sequence of SEQ ID NO:8.

49. An antibody-drug conjugate having formula (I): 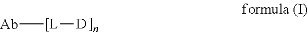
wherein in formula (I):
Ab comprises an antibody or antibody-binding fragment, wherein the antibody or binding fragment thereof specifically binds CUB Domain-Containing Protein-1 (CDCP1);
n is 1;
L-D has the formula: 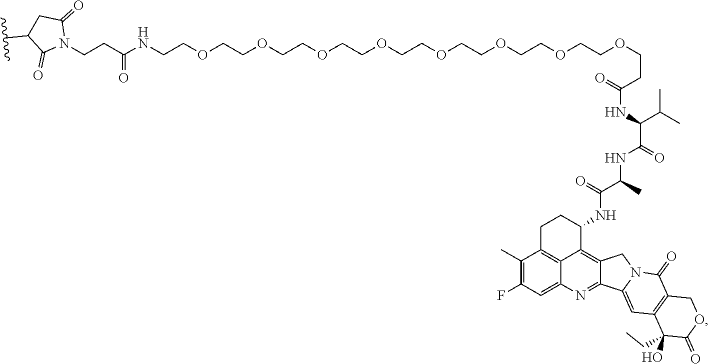 [SMILES: *C1CC(=O)N(CCC(=O)NCCOCCOCCOCCOCCOCCOCCOCCOCCC(=O)N[C@H](C(=O)N[C@@H](C)C(=O)N[C@H]2CCc3c(C)c(F)cc4nc5c(c2c34)Cn2c-5cc3c(c2=O)COC(=O)[C@]3(O)CC)C(C)C)C1=O]
wherein the antibody or binding fragment thereof comprises:
(i) a heavy chain variable region (VH) that comprises:
(a) a VH complementarity determining region one (CDRH1) comprising and/or consisting of the amino acid sequence of SEQ ID NO: 12,
(b) a VH complementarity determining region two (CDRH2) comprising and/or consisting of the amino acid sequence of SEQ ID NO: 13, and
(c) a VH complementarity determining region three (CDRH3) comprising and/or consisting of the amino acid sequence of SEQ ID NO: 14,
and (ii) a light chain variable region (VL) that comprises:
(a) a VL complementarity determining region one (CDRL1) comprising and/or consisting of the amino acid sequence of SEQ ID NO: 6,
(b) a VL complementarity determining region two (CDRL2) comprising and/or consisting of the amino acid sequence of SEQ ID NO: 7, and
(c) a VL complementarity determining region three (CDRL3) comprising and/or consisting of the amino acid sequence of SEQ ID NO:8.

50. An antibody-drug conjugate having formula (I): 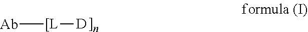
wherein in formula (I):
Ab comprises an antibody or antibody-binding fragment, wherein the antibody or binding fragment thereof specifically binds CUB Domain-Containing Protein-1 (CDCP1);
n is 4;
L-D has the formula: 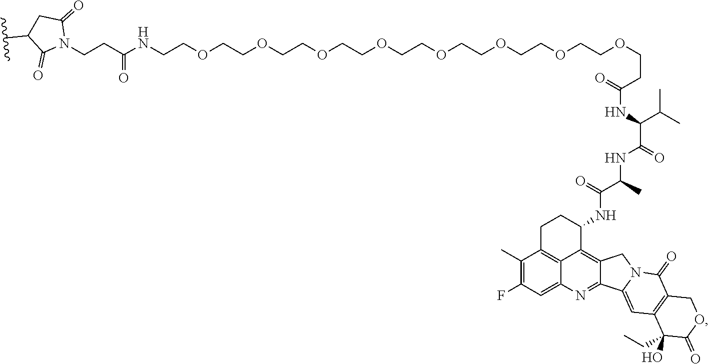 [SMILES: *C1CC(=O)N(CCC(=O)NCCOCCOCCOCCOCCOCCOCCOCCOCCC(=O)N[C@H](C(=O)N[C@@H](C)C(=O)N[C@H]2CCc3c(C)c(F)cc4nc5c(c2c34)Cn2c-5cc3c(c2=O)COC(=O)[C@]3(O)CC)C(C)C)C1=O]
wherein the antibody or binding fragment thereof comprises:
(i) a heavy chain variable region (VH) that comprises:
(a) a VH complementarity determining region one (CDRH1) comprising and/or consisting of the amino acid sequence of SEQ ID NO: 12,
(b) a VH complementarity determining region two (CDRH2) comprising and/or consisting of the amino acid sequence of SEQ ID NO: 13, and
(c) a VH complementarity determining region three (CDRH3) comprising and/or consisting of the amino acid sequence of SEQ ID NO: 14,
and (ii) a light chain variable region (VL) that comprises:
(a) a VL complementarity determining region one (CDRL1) comprising and/or consisting of the amino acid sequence of SEQ ID NO: 6,
(b) a VL complementarity determining region two (CDRL2) comprising and/or consisting of the amino acid sequence of SEQ ID NO: 7, and
(c) a VL complementarity determining region three (CDRL3) comprising and/or consisting of the amino acid sequence of SEQ ID NO:8.

51. An antibody-drug conjugate having formula (I): 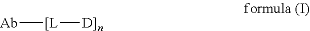
wherein in formula (I):
Ab comprises an antibody or antibody-binding fragment, wherein the antibody or binding fragment thereof specifically binds CUB Domain-Containing Protein-1 (CDCP1);
n is 8;
L-D has the formula: 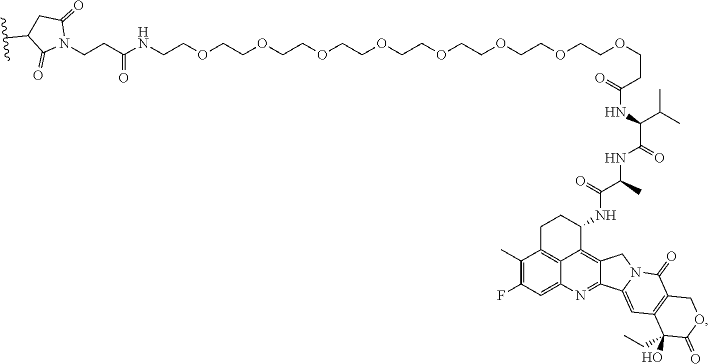 [SMILES: *C1CC(=O)N(CCC(=O)NCCOCCOCCOCCOCCOCCOCCOCCOCCC(=O)N[C@H](C(=O)N[C@@H](C)C(=O)N[C@H]2CCc3c(C)c(F)cc4nc5c(c2c34)Cn2c-5cc3c(c2=O)COC(=O)[C@]3(O)CC)C(C)C)C1=O]
wherein the antibody or binding fragment thereof comprises:
(i) a heavy chain variable region (VH) that comprises:
(a) a VH complementarity determining region one (CDRH1) comprising and/or consisting of the amino acid sequence of SEQ ID NO: 12,
(b) a VH complementarity determining region two (CDRH2) comprising and/or consisting of the amino acid sequence of SEQ ID NO: 13, and
(c) a VH complementarity determining region three (CDRH3) comprising and/or consisting of the amino acid sequence of SEQ ID NO: 14,
and (ii) a light chain variable region (VL) that comprises:
(a) a VL complementarity determining region one (CDRL1) comprising and/or consisting of the amino acid sequence of SEQ ID NO: 6,
(b) a VL complementarity determining region two (CDRL2) comprising and/or consisting of the amino acid sequence of SEQ ID NO: 7, and
(c) a VL complementarity determining region three (CDRL3) comprising and/or consisting of the amino acid sequence of SEQ ID NO:8.

52. A method of treating a cancer comprising administering to a subject in need thereof a therapeutically effective amount of the antibody-drug conjugate of any one of claims 33-51.
